(19) United States
(12) Patent Application Publication
(10) Pub. No.: US 2025/0152601 A1
(43) Pub. Date: May 15, 2025

(54) Compositions and Methods for the Treatment of Sexual Dysfunctions

(71) Applicant: The Jonathan Hurt Living Trust, Austin, TX (US)

(72) Inventor: Jonathan Dale Hurt, Austin, TX (US)

(73) Assignee: The Jonathan Hurt Living Trust, Austin, TX (US)

(21) Appl. No.: 18/839,364

(22) PCT Filed: Jan. 23, 2023
(86) PCT No.: PCT/US23/61108

§ 371 Date: Aug. 16, 2024

Related U.S. Application Data
(60) Provisional application No. 63/313,032, filed on Feb. 23, 2022.

(51) Int. Cl. A61K 31/55; A61K 9/00; A61K 31/198; A61P 15/10

(52) U.S. Cl. CPC A61K 31/55; A61K 9/0053; A61K 31/198; A61P 15/10

(57) ABSTRACT

Disclosed herein are compositions and methods for treating sexual dysfunctions including a reduction and/or an absence of tactile sexual function in the genitals, genital numbness, delayed ejaculation and/or failure to ejaculate, premature ejaculation, anorgasmia, decreased libido, erectile dysfunction, sexual anhedonia, post-selective serotonin reuptake inhibitor sexual dysfunction, or a combination thereof.

### PRIORITY CLAIM

This patent application claims the benefit of priority under U.S.C. § 119(e) to U.S. Provisional Application Ser. No. 63/313,032 entitled “Compositions and Methods for the Treatment of Sexual Dysfunctions” filed on Feb. 23, 2022, the entire contents of which are incorporated herein by reference.

### STATEMENT REGARDING FEDERALLY SPONSORED RESEARCH OR DEVELOPMENT

Not applicable.

### FIELD OF THE INVENTION

The present disclosures generally relate to compositions and methods for treating sexual dysfunctions including a reduction of sexual sensation in the genitals, delayed ejaculation and/or failure to ejaculate, premature ejaculation, anorgasmia, and/or sexual anhedonia.

### BACKGROUND OF THE INVENTION

In the year 1999 the inventor was prescribed the drug paroxetine (sold under the name Paxil), a selective serotonin reuptake inhibitor (“SSRI”), and began orally ingesting paroxetine per the written prescription's instructions. For at least several years before ingesting paroxetine the inventor was also prescribed and ingested by oral administration finasteride (sold under the name Propecia), an inhibitor of 5Alpha-reductase that reduces production of dihydrotestosterone, per the written prescription's instructions. Sexual sensation herein is a tactile sexual sensation in the genitals that is weaker than being pleasurable during one or more of the sexual responses of desire, excitement, plateau, orgasm, and resolution. Sexual pleasure herein is a tactile sexual sensation in the genitals that is pleasurable during one or more of the sexual responses of excitement, plateau, orgasm, and resolution. Sexual sensation and/or sexual pleasure is referred to herein as tactile sexual function (abbreviated herein as “TSF”). Finasteride moderately reduced TSF for the inventor while ingesting the drug, but full TSF returned rapidly upon discontinuing ingesting finasteride during intermittent periods of non-use during the years before ingesting paroxetine. When both paroxetine and finasteride were ingested, complete lack of TSF began and remained during use of both drugs. Paroxetine was ingested for some months (likely less than a year). After discontinuing paroxetine use, absence of TSF continued much to most of the time, though some TSF of non-pleasant tactile sensation to modestly pleasurable intensity would sometimes occur, and this condition of reduced TSF is referred to herein as a substantive reduction of sexual sensation (“SRSS”) relative to the normal TSF before ingesting either paroxetine and/or finasteride. Finasteride use was discontinued sometime between February 2003 and Jan. 1, 2011, from available prescription records, and the SRSS continued with no noticeable improvement. Ejaculation does occur with physical stimulation, though the ease to achieve ejaculation was also reduced due to the SRSS. Non-sexual tactile sensation (e.g., sensation of heat, cold, pressure, pain, etc.) in the genitals remained normal; and all other aspects of sexual function (e.g., sex drive/libido) remained the same relative to before SRSS began. Other people (e.g., men, women) also have SRSS and/or have another sexual dysfunction associated with current or past ingestion of some pharmaceuticals, including SSRIs and finasteride [Hieronymus, F. et al. Acta Neuropsychiatr 2018 30(5):244-250; Bahrick, A. S. The Open Psychology Journal 2008 1:42-50; Bahrick, A. S. 2006 American Society for the Advancement of Pharmacotherapy. Tablet 7(3):2-3; “Minutes of PRAC meeting of 13-16 May 2019: Signal of persistent sexual dysfunction after drug withdrawal” European Medicines Agency. 14 Jun. 2019; Pharmacovigilance Risk Assessment Committee (PRAC) (11 Jun. 2019). “New product information wording—Extracts from PRAC recommendations on signals” European Medicines Agency. EMA/PRAC/265221/2019; Taylor, M. J. et al. “Strategies for managing sexual dysfunction induced by antidepressant medication”. The Cochrane Database of Systematic Reviews. (May 2013) 5 (5):CD003382; Healy, D. Epidemiology and Psychiatric Sciences. 2020 29: e55; Reisman, Y. Sexual Medicine Reviews. 2017 5 (4):429-433].

### DESCRIPTION OF THE RELATED ART

See the information disclosure statement, if any.

### BRIEF SUMMARY OF THE EMBODIMENTS

The embodiments of the invention provide a composition for treating a reduced amount of tactile sexual function, comprising: at least one positive GABAergic treatment substance and at least one positive cysteineic treatment substance in an effective amount to achieve pleasurable tactile sexual function just prior to the beginning of orgasm, just after the beginning of orgasm, or both. In some embodiments, the at least one positive GABAergic treatment substance comprises a treatment substance that promotes the activity of a GABA receptor, a treatment substance that inhibits GABA transaminase, a treatment substance that activates glutamic acid decarboxylase, a treatment substance that inhibits a GAT transporter, or a combination thereof, after ingesting the treatment substance. In other embodiments, the at least one positive GABAergic treatment substance promotes the activity of a GABA receptor after ingesting the treatment substance. In further embodiments, the treatment substance that promotes the activity of a GABA receptor after ingesting the treatment substance comprises a treatment substance that increases the amount of a GABA receptor agonist, a treatment substance that increases the amount of a GABA receptor positive allosteric modulator, a treatment substance that activates a KCC2 transporter, a treatment substance that activates a TRPV1 receptor, or a combination thereof. In some embodiments, the treatment substance that increases the amount of a GABA receptor agonist comprises homotaurine, at least one precursor of GABA, at least one GABA prodrug, GABA, a Withania somnifera preparation, or a combination thereof.

In some aspects, the at least one positive GABAergic treatment substance increases the amount of a GABA receptor agonist after ingesting the treatment substance. In some facets, the at least one positive GABAergic treatment substance comprises homotaurine. In other facets, the amount of homotaurine ingested is about 0.7 mg per kilogram body weight to about 3.2 mg per kilogram body weight. In some aspect, the at least one positive GABAergic treatment substance comprises at least one precursor of GABA. In other aspects, precursor of GABA comprises one, two, or three branched chain amino acids. In some aspects, the branched chain amino acids comprise valine, leucine, isoleucine, or a combination thereof. In some facets, the at least one positive GABAergic treatment substance comprises three branched chain amino acids. In other facets, the three branched chain amino acids are about 60.7 mg per kilogram body weight to about 99.2 mg per kilogram body weight. In some aspect, the at least one positive GABAergic treatment substance comprises valine. In other aspects, the valine is about 10.1 mg per kilogram body weight to about 116.1 mg per kilogram body weight. In some aspects, the at least one positive GABAergic treatment substance comprises leucine. In other aspects, the leucine is about 10.1 mg per kilogram body weight to about 116.1 mg per kilogram body weight. In some aspects, the at least one positive GABAergic treatment substance comprises isoleucine. In other aspects, the isoleucine is about 10.1 mg per kilogram body weight to about 48.4 mg per kilogram body weight. In some aspects, the at least one positive GABAergic treatment substance comprises at least one GABA prodrug. In other aspects, the at least one GABA prodrug comprises nicotinoyl-GABA. In some facets, the nicotinoyl-GABA is about 1.5 mg per kilogram body weight to about 8.1 mg per kilogram body weight. In some aspects, the at least one positive GABAergic treatment substance comprises GABA. In other aspects, the GABA is about 22.1 mg per kilogram body weight to about 24.2 mg per kilogram body weight. In some aspects, the at least one positive GABAergic treatment substance comprises a Withania somnifera Preparation. In other aspects, the Withania somnifera Preparation is about 4.4 mg per kilogram body weight to about 4.8 mg per kilogram body weight.

In some embodiments, the at least one positive GABAergic treatment substance comprises at least one treatment substance that increases the amount of a GABA receptor positive allosteric modulator after ingesting the treatment substance. In some embodiments, the treatment substance that increases the amount of a GABA receptor positive allosteric modulator comprises a Boswellia serrata preparation, a Crocus sativus preparation, a Piper methysticum preparation, theanine, baicalin preparation, or a combination thereof. In some aspects, the at least one positive GABAergic treatment substance comprises a Boswellia serrata preparation. In other aspects, the Boswellia serrata preparation is about 3.7 mg per kilogram body weight to about 4.0 mg per kilogram body weight. In some aspects, the at least one positive GABAergic treatment substance comprises a Crocus sativus preparation. In other aspects, the Crocus sativus preparation is about 5.2 mg per kilogram body weight to about 5.7 mg per kilogram body weight. In some aspects, the at least one positive GABAergic treatment substance comprises a Piper methysticum preparation. In other aspects, the Piper methysticum preparation is about 4.3 mg per kilogram body weight to about 20.6 mg per kilogram body weight. In some aspects, the at least one positive GABAergic treatment substance comprises theanine. In other aspects, the theanine is about 5.9 mg per kilogram body weight to about 9.7 mg per kilogram body weight. In some aspects, the at least one positive GABAergic treatment substance comprises a baicalin preparation. In other aspects, the baicalin preparation is about 4.0 mg per kilogram body weight to about 13.1 mg per kilogram body weight.

In some embodiments, the at least one positive GABAergic treatment substance comprises at least one treatment substance that activates a KCC2 transporter after ingesting the treatment substance. In some aspects, the at least one least one treatment substance that activates a KCC2 transporter comprises trans-resveratrol, piperine, or a combination thereof. In other aspects, the at least one positive GABAergic treatment substance comprises at least one treatment substance that activates a KCC2 transporter after ingesting the treatment substance comprises at least one treatment substance that activates a TRPV1-receptor. In some aspects, the at least one positive GABAergic treatment substance comprises at least one treatment substance that activates a TRPV1-receptor. In some facets, the at least one treatment substance that activates a TRPV1-receptor comprises piperine. In some aspects, the at least one positive GABAergic treatment substance comprises trans-resveratrol. In other aspects, the trans-resveratrol is about 7.4 mg per kilogram body weight to about 8.1 mg per kilogram body weight. In other aspects, the at least one positive GABAergic treatment substance comprises piperine. In some facets, the piperine is about 0.22 mg per kilogram body weight to about 0.24 mg per kilogram body weight. In other aspects, the at least one positive GABAergic treatment substance comprises a Vanilla planifolia preparation. In other facets, the Vanilla planifolia preparation is about 0.016 mL per kilogram body weight to about 0.015 mL per kilogram body weight.

In some embodiments, the at least one positive GABAergic treatment substance comprises at least one treatment substance that inhibits GABA transaminase after ingesting the treatment substance. In other embodiments, the least one treatment substance that inhibits GABA transaminase comprises a Melissa officinalis preparation. In some aspects, the at least one positive GABAergic treatment substance comprises a Melissa officinalis preparation. In other aspects, the Melissa officinalis preparation is about 16.5 mg per kilogram body weight to about 29.0 mg per kilogram body weight.

In some embodiments, the at least one positive GABAergic treatment substance comprises at least one treatment substance that activates glutamic acid decarboxylase after ingesting the treatment substance. In other embodiments, the least one treatment substance that activates glutamic acid decarboxylase comprises a Valeriana officinalis preparation. In certain aspects, the at least one positive GABAergic treatment substance comprises a Valeriana officinalis preparation. In other aspects, the Valeriana officinalis preparation is about 14.7 mg per kilogram body weight to about 35.5 mg per kilogram body weight.

In some embodiments, the at least one positive GABAergic treatment substance comprises at least one treatment substance that inhibits a GAT transporter after ingesting the treatment substance. In other embodiments, the at least one treatment substance that inhibits a GAT transporter comprises taurine. In certain aspects, the at least one positive GABAergic treatment substance comprises taurine. In other aspects, the taurine is about 14.7 mg per kilogram body weight to about 16.1 mg per kilogram body weight.

In some embodiments, the at least one positive cysteineic treatment substance comprises comprises N-acetyl-cysteine, cysteine, cystine, or a combination thereof. In other aspects, the at least one positive cysteineic treatment substance comprises N-acetyl-cysteine. In some aspects, the N-acetyl-cysteine is about 3.7 mg per kilogram body weight to about 32.3 mg per kilogram body weight. In other aspects, the at least one positive cysteineic treatment substance comprises cysteine, cystine, or both. In certain facets, the cysteine, cystine, or both is about 7.4 mg per kilogram body weight to about 8.1 mg per kilogram body weight.

In particular embodiments, the at least one positive GABAergic treatment substance also is a negative glutamatergic treatment substance, the at least one positive cysteineic treatment substance, or a combination thereof, are at least one negative glutamatergic treatment substance. In some embodiments, the positive GABAergic treatment substance also is a negative glutamatergic treatment substance. In some aspects, the negative glutamatergic treatment substance comprises magnesium threonate. In other aspects, the magnesium threonate is about 9.8 mg per kilogram body weight to about 84.0 mg per kilogram body weight. In some facets, the negative glutamatergic treatment substance comprises positive cysteineic treatment substance.

In some embodiments, the composition further comprises at least one positive cholinergic treatment substance. In other embodiments, the at least one positive cholinergic treatment substance comprises at least one acetylcholinesterase inhibitor, at least one acetylcholine precursor, or a combination thereof. In some aspects, the at least one at least one positive cholinergic treatment substance comprises at least one acetylcholinesterase inhibitor. In some facets, the at least one acetylcholinesterase inhibitor comprises a Panax genus preparation, huperzine A, galantamine HBr, or a combination thereof. In other facets, the at least one positive cholinergic treatment substance comprises a Panax genus preparation. In certain facets, the Panax genus preparation is about 8.8 mg per kilogram body weight to about 9.7 mg per kilogram body weight. In some facets, the at least one positive cholinergic treatment substance comprises huperzine A. In other facets, the huperzine A is about 0.00074 mg per kilogram body weight to about 0.00161 mg per kilogram body weight. In some aspects, the at least one positive cholinergic treatment substance comprises galantamine HBr. In other aspects, the galantamine HBr is about 0.59 mg per kilogram body weight to about 0.194 mg per kilogram body weight. In some aspects, the at least one positive cholinergic treatment substance comprises at least one acetylcholine precursor. In other aspects, the at least one acetylcholine precursor comprises alpha-glycerophosphocholine, centrophenoxine, or a combination thereof. In some aspects, the at least one positive cholinergic treatment substance comprises alpha-glycerophosphocholine. In other aspects, the alpha-glycerophosphocholine is about 4.4 mg per kilogram body weight to about 4.8 mg per kilogram body weight. In some aspects, the at least one positive cholinergic treatment substance comprises centrophenoxine. In other aspects, the centrophenoxine is about 7.4 mg per kilogram body weight to about 8.1 mg per kilogram body weight.

In some embodiments, the composition further comprises at least one positive cannabinoidergic treatment substance. In other embodiments, the at least one positive cannabinoidergic treatment substance comprises a Syzygium aromaticum preparation, calcium pyruvate, oleamide, or a combination thereof. In some aspects, the at least one positive cannabinoidergic treatment substance comprises a Syzygium aromaticum preparation. In other aspects, the Syzygium aromaticum preparation is about 4.4 mg per kilogram body weight to about 4.8 mg per kilogram body weight. In some aspects, the at least one positive cannabinoidergic treatment substance comprises calcium pyruvate. In other aspects, the calcium pyruvate is about 13.2 mg per kilogram body weight to about 29.0 mg per kilogram body weight. In some aspects, the at least one positive cannabinoidergic treatment substance comprises oleamide. In other aspects, the oleamide is about 0.7 mg per kilogram body weight to about 3.2 mg per kilogram body weight.

In some embodiments, the composition further comprises at least one positive nitroergic treatment substance. In other embodiments, the at least one positive nitroergic treatment substance comprises norvaline, icariin, or a combination thereof. In some aspects, the at least one positive nitroergic treatment substance comprises norvaline. In other aspects, the norvaline is about 3.9 mg per kilogram body weight to about 35.0 mg per kilogram body weight. In some aspects, the at least one positive nitroergic treatment substance comprises icariin. In other aspects, the icariin is about 0.88 mg per kilogram body weight to about 0.97 mg per kilogram body weight.

In some embodiments, the composition further comprises at least one negative adenosinergic treatment substance. In other embodiments, the at least one negative adenosinergic treatment substance comprises theobromine, caffeine, or a combination thereof. In some aspects, the at least one negative adenosinergic treatment substance comprises theobromine. In other aspects, the theobromine is about 17.6 mg per kilogram body weight to about 19.4 mg per kilogram body weight. In some aspects, the at least one negative adenosinergic treatment substance comprises caffeine. In other aspects, the caffeine is about 1.5 mg per kilogram body weight to about 3.2 mg per kilogram body weight.

In some embodiments, the composition further comprises at least one positive glycinergic treatment substance. In other embodiments, the at least one positive glycinergic treatment substance comprises glycine, pramiracetam, or a combination thereof. In some aspects, the at least one positive glycinergic treatment substance comprises glycine. In other aspects, the glycine is about 14.7 mg per kilogram body weight to about 64.5 mg per kilogram body weight. In some aspects, the at least one positive glycinergic treatment substance comprises pramiracetam. In other aspects, the pramiracetam is about 3.7 mg per kilogram body weight to about 10.1 mg per kilogram body weight.

In some embodiments, the composition further comprises at least one negative glutamatergic treatment substance. In some aspects, the at least one negative glutamatergic treatment substance comprises noopept. In some facets, the noopept is about 0.4 mg per kilogram body weight to about 1.5 mg per kilogram body weight.

In some embodiments, the at least one positive GABAergic treatment substance comprises valine and the at least one positive cysteineic treatment substance comprises N-acetyl-cysteine. In some embodiments, the at least one positive GABAergic treatment substance comprises valine and magnesium threonate and the at least one positive cysteineic treatment substance comprises N-acetyl-cysteine. In some embodiments, the at least one positive GABAergic treatment substance comprises valine and magnesium threonate and the at least one positive cysteineic treatment substance comprises N-acetyl-cysteine, and wherein the composition further comprises at least one acetylcholinesterase inhibitor selected from huperzine A, galantamine HBr, and a Panax genus preparation. In some embodiments, the at least one positive GABAergic treatment substance comprises valine and magnesium threonate and the at least one positive cysteineic treatment substance comprises N-acetyl-cysteine, and wherein the composition further comprises at least one positive glycinergic treatment substance selected from glycine and pramiracetam. In some embodiments, the at least one positive GABAergic treatment substance comprises valine and magnesium threonate and the at least one positive cysteineic treatment substance comprises N-acetyl-cysteine, and wherein the composition further comprises at least one positive glycinergic treatment substance selected from glycine and pramiracetam, and wherein the composition further comprises at least one positive acetylcholinesterase inhibitor selected from huperzine A, galantamine HBr, and a Panax genus preparation. In some embodiments, the at least one positive GABAergic treatment substance comprises nicotinoyl-GABA and the at least one positive cysteineic treatment substance comprises N-acetyl-cysteine. In some embodiments, the at least one positive GABAergic treatment substance comprises nicotinoyl-GABA and magnesium threonate and the at least one positive cysteineic treatment substance comprises N-acetyl-cysteine. In some embodiments, the at least one positive GABAergic treatment substance comprises nicotinoyl-GABA and magnesium threonate and the at least one positive cysteineic treatment substance comprises N-acetyl-cysteine, and wherein the composition further comprises at least one acetylcholinesterase inhibitor selected from huperzine A, galantamine HBr, and a Panax genus preparation. In some embodiments, the at least one positive GABAergic treatment substance comprises nicotinoyl-GABA and magnesium threonate and the at least one positive cysteineic treatment substance comprises N-acetyl-cysteine, and wherein the composition further comprises at least one positive glycinergic treatment substance selected from glycine and pramiracetam. In some embodiments, the at least one positive GABAergic treatment substance comprises nicotinoyl-GABA and magnesium threonate and the at least one positive cysteineic treatment substance comprises N-acetyl-cysteine, and wherein the composition further comprises at least one positive glycinergic treatment substance selected from glycine and pramiracetam, and wherein the composition further comprises at least one positive acetylcholinesterase inhibitor selected from huperzine A, galantamine HBr, and a Panax genus preparation. In some embodiments, the at least one positive GABAergic treatment substance comprises valine and nicotinoyl-GABA and the at least one positive cysteineic treatment substance comprises N-acetyl-cysteine. In some embodiments, the at least one positive GABAergic treatment substance comprises valine, nicotinoyl-GABA and magnesium threonate and the at least one positive cysteineic treatment substance comprises N-acetyl-cysteine. In some embodiments, the at least one positive GABAergic treatment substance comprises valine, nicotinoyl-GABA and magnesium threonate and the at least one positive cysteineic treatment substance comprises N-acetyl-cysteine, and wherein the composition further comprises at least one acetylcholinesterase inhibitor selected from huperzine A, galantamine HBr, and a Panax genus preparation. In some embodiments, the at least one positive GABAergic treatment substance comprises valine, nicotinoyl-GABA and magnesium threonate and the at least one positive cysteineic treatment substance comprises N-acetyl-cysteine, and wherein the composition further comprises at least one positive glycinergic treatment substance selected from glycine and pramiracetam. In some embodiments, the at least one positive GABAergic treatment substance comprises valine, nicotinoyl-GABA and magnesium threonate and the at least one positive cysteineic treatment substance comprises N-acetyl-cysteine, and wherein the composition further comprises at least one positive glycinergic treatment substance selected from glycine and pramiracetam and the composition further comprises at least one positive acetylcholinesterase inhibitor selected from huperzine A, galantamine HBr, and a Panax genus preparation.

In other embodiments, the at least one positive GABAergic treatment substance consists essentially of valine and at least one positive cysteineic treatment substance consists essentially of N-acetyl-cysteine. In other embodiments, the at least one positive GABAergic treatment substance consists essentially of valine and at least one positive cysteineic treatment substance consists essentially of N-acetyl-cysteine, wherein the valine is about 10.1 mg per kilogram body weight to about 116.1 mg per kilogram body weight, and wherein the N-acetyl-cysteine is about 3.7 mg per kilogram body weight to about 32.3 mg per kilogram body weight. In other embodiments, the at least one positive GABAergic treatment substance consists essentially of a combination of valine and magnesium threonate, and the at least one positive cysteineic treatment substance consists essentially of N-acetyl-cysteine. In other embodiments, the at least one positive GABAergic treatment substance consists essentially of a combination of valine and magnesium threonate, and the at least one positive cysteineic treatment substance consists essentially of N-acetyl-cysteine, wherein the valine is about 10.1 mg per kilogram body weight to about 116.1 mg per kilogram body weight, wherein the magnesium threonate is about 9.8 mg per kilogram body weight to about 84.0 mg per kilogram body weight, and wherein the N-acetyl-cysteine is about 3.7 mg per kilogram body weight to about 32.3 mg per kilogram body weight. In other embodiments, the at least one positive GABAergic treatment substance consists essentially of a combination of valine and magnesium threonate and the at least one positive cysteineic treatment substance consists essentially of N-acetyl-cysteine, further in combination with an acetylcholinestase inhibitor consisting essentially of galantamine HBr. In other embodiments, the at least one positive GABAergic treatment substance consists essentially of a combination of valine and magnesium threonate and the at least one positive cysteineic treatment substance consists essentially of N-acetyl-cysteine, further in combination with an acetylcholinestase inhibitor consisting essentially of galantamine HBr, wherein the valine is about 10.1 mg per kilogram body weight to about 116.1 mg per kilogram body weight, wherein the magnesium threonate is about 9.8 mg per kilogram body weight to about 84.0 mg per kilogram body weight, wherein the N-acetyl-cysteine is about 3.7 mg per kilogram body weight to about 32.3 mg per kilogram body weight, and wherein the galantamine HBr is about 0.59 mg per kilogram body weight to about 0.194 mg per kilogram body weight. In other embodiments, the at least one positive GABAergic treatment substance consists essentially of nicotinoyl-GABA and the at least one positive cysteineic treatment substance consists essentially of N-acetyl-cysteine. In other embodiments, the at least one positive GABAergic treatment substance consists essentially of nicotinoyl-GABA and the at least one positive cysteineic treatment substance consists essentially of N-acetyl-cysteine, wherein the nicotinoyl-GABA is about 1.5 mg per kilogram body weight to about 8.1 mg per kilogram body weight, and wherein the N-acetyl-cysteine is about 3.7 mg per kilogram body weight to about 32.3 mg per kilogram body weight. In other embodiments, the at least one positive GABAergic treatment substance consists essentially of a combination of nicotinoyl-GABA and magnesium threonate and the at least one positive cysteineic treatment substance consists essentially of N-acetyl-cysteine. In other embodiments, the at least one positive GABAergic treatment substance consists essentially of a combination of nicotinoyl-GABA and magnesium threonate and the at least one positive cysteineic treatment substance consists essentially of N-acetyl-cysteine, wherein the nicotinoyl-GABA is about 1.5 mg per kilogram body weight to about 8.1 mg per kilogram body weight, wherein the magnesium threonate is about 9.8 mg per kilogram body weight to about 84.0 mg per kilogram body weight, and wherein the N-acetyl-cysteine is about 3.7 mg per kilogram body weight to about 32.3 mg per kilogram body weight. In other embodiments, the at least one positive GABAergic treatment substance consists essentially of a combination of valine and magnesium threonate and the at least one positive cysteineic treatment substance consists essentially of N-acetyl-cysteine, further in combination with an acetylcholinestase inhibitor consisting essentially of galantamine HBr. In other embodiments, the at least one positive GABAergic treatment substance consists essentially of a combination of valine and magnesium threonate and the at least one positive cysteineic treatment substance consists essentially of N-acetyl-cysteine, further in combination with an acetylcholinestase inhibitor consisting essentially of galantamine HBr, wherein the valine is about 10.1 mg per kilogram body weight to about 116.1 mg per kilogram body weight, wherein the magnesium threonate is about 9.8 mg per kilogram body weight to about 84.0 mg per kilogram body weight, wherein the N-acetyl-cysteine is about 3.7 mg per kilogram body weight to about 32.3 mg per kilogram body weight, and wherein the galantamine HBr is about 0.59 mg per kilogram body weight to about 0.194 mg per kilogram body weight. In other embodiments, the at least one positive GABAergic treatment substance consists essentially of a combination of nicotinoyl-GABA, valine and magnesium threonate and the at least one positive cysteineic treatment substance consists essentially of N-acetyl-cysteine. In other embodiments, the at least one positive GABAergic treatment substance consists essentially of a combination of nicotinoyl-GABA, valine and magnesium threonate and the at least one positive cysteineic treatment substance consists essentially of N-acetyl-cysteine, wherein the nicotinoyl-GABA is about 1.5 mg per kilogram body weight to about 8.1 mg per kilogram body weight, wherein the valine is about 10.1 mg per kilogram body weight to about 116.1 mg per kilogram body weight, wherein the magnesium threonate is about 9.8 mg per kilogram body weight to about 84.0 mg per kilogram body weight, and wherein the N-acetyl-cysteine is about 3.7 mg per kilogram body weight to about 32.3 mg per kilogram body weight.

In some embodiments, a composition further excludes at least one negative GABAergic treatment substance in an effective amount to increase the time to ejaculation, reduces tactile sexual function, or both; wherein the negative GABAergic treatment substance is selected from the group of a carbonic anhydrase inhibitor and a positive glutamatergic treatment substance; wherein the positive glutamatergic treatment substance activates at least one receptor that activates at least one ion channel, and wherein the receptor is selected from a NMDA receptor and an AMPA receptor. In certain aspects, the positive glutamatergic treatment substance comprises NMDA.

Some embodiments provide a composition for treating a reduced amount of tactile sexual function, comprising: at least one positive GABAergic treatment substance and at least one positive cysteineic treatment substance in an effective amount to achieve pleasurable tactile sexual function just before the beginning of orgasm, just after the beginning of orgasm, or both; wherein the at least one positive GABAergic treatment substance consists essentially of valine and at least one positive cysteineic treatment substance consists essentially of N-acetyl-cysteine. In some aspects, the valine is about 10.1 mg per kilogram body weight to about 116.1 mg per kilogram body weight, and the N-acetyl-cysteine is about 3.7 mg per kilogram body weight to about 32.3 mg per kilogram body weight.

Some embodiments provide a composition for treating a reduced amount of tactile sexual function, comprising: at least one positive GABAergic treatment substance and at least one positive cysteineic treatment substance in an effective amount to achieve pleasurable tactile sexual function just before the beginning of orgasm, just after the beginning of orgasm, or both; wherein the at least one positive GABAergic treatment substance consists essentially of valine the at least one positive cysteineic treatment substance consists essentially of N-acetyl-cysteine, further in combination with an acetylcholinestase inhibitor consisting essentially of galantamine HBr. In certain aspects, the valine is about 10.1 mg per kilogram body weight to about 116.1 mg per kilogram body weight, and the N-acetyl-cysteine is about 3.7 mg per kilogram body weight to about 32.3 mg per kilogram body weight, and the galantamine HBr is about 0.59 mg per kilogram body weight to about 0.194 mg per kilogram body weight.

Some embodiments provide a composition for treating a reduced amount of tactile sexual function, comprising: at least one positive GABAergic treatment substance and at least one positive cysteineic treatment substance in an effective amount to achieve pleasurable tactile sexual function just before the beginning of orgasm, just after the beginning of orgasm, or both; wherein the at least one positive GABAergic treatment substance consists essentially of nicotinoyl-GABA and at least one positive cysteineic treatment substance consists essentially of N-acetyl-cysteine. In some aspects, the nicotinoyl-GABA is about 1.5 mg per kilogram body weight to about 8.1 mg per kilogram body weight, and the N-acetyl-cysteine is about 3.7 mg per kilogram body weight to about 32.3 mg per kilogram body weight.

Some embodiments provide a composition for treating a reduced amount of tactile sexual function, comprising: at least one positive GABAergic treatment substance and at least one positive cysteineic treatment substance in an effective amount to achieve pleasurable tactile sexual function just before the beginning of orgasm, just after the beginning of orgasm, or both; wherein the at least one positive GABAergic treatment substance consists essentially of nicotinoyl-GABA and at least one positive cysteineic treatment substance consists essentially of N-acetyl-cysteine, further in combination with an acetylcholinestase inhibitor consisting essentially of galantamine HBr. In certain aspects, the nicotinoyl-GABA is about 1.5 mg per kilogram body weight to about 8.1 mg per kilogram body weight, the N-acetyl-cysteine is about 3.7 mg per kilogram body weight to about 32.3 mg per kilogram body weight, and the galantamine HBr is about 0.59 mg per kilogram body weight to about 0.194 mg per kilogram body weight.

Some embodiments provide a method for treating a reduced amount of tactile sexual function, comprising: ingesting a first treatment substance comprising at least one positive GABAergic treatment substance and at least one positive cysteineic treatment substance in an effective amount to achieve pleasurable tactile sexual function just prior to the beginning of orgasm, just after the beginning of orgasm, or both, and wherein tactile sexual stimulation occurs between about 5 minutes to about 120 minutes after ingesting the first treatment substance. In some aspects the method further comprises ingesting at least one additional treatment substance in accordance with claim 1 within 5 to 60 minutes of ingesting the first treatment substance, and wherein tactile sexual stimulation occurs between about 5 minutes to about 120 minutes after ingesting the at least one additional treatment substance.

Some embodiments providing a method for determining the duration of effect and/or efficacy of an ingested treatment substance on a neurotransmission system, comprising obtaining an individual having a sexual dysfunction, ingesting at least one treatment substance, and measuring the duration and/or intensity of tactile sexual sensation to determine the duration of effect and/or efficacy of the at least one treatment substance. In some aspects, the at least one treatment substance comprises at least one positive GABAergic treatment substance, at least one positive cysteineic treatment substance, at least one positive cholinergic treatment substance, or a combination thereof.

Some embodiments provide a composition for treating delayed ejaculation, delayed orgasm, failure to ejaculate, and/or failure to orgasm, comprising: at least one positive cholinergic treatment substance in an effective amount to decrease the time to ejaculation and/or orgasm. Other embodiments provide a method for treating delayed ejaculation, delayed orgasm, failure to ejaculate, and/or failure to orgasm, comprising: ingesting at least one positive cholinergic treatment substance in an effective amount to to decrease the time to ejaculation and/or orgasm.

Some embodiments provide a composition for treating premature ejaculation, comprising: at least one negative GABAergic treatment substance in an effective amount to increase the time to ejaculation, selected from the group of a carbonic anhydrase inhibitor and NMDA, and wherein tactile sexual stimulation occurs between about 5 minutes to about 120 minutes after ingesting the at least one negative GABAergic treatment substance. In certain aspects, the positive glutamatergic treatment substance comprises NMDA.

Certain embodiments provide a kit for treating a reduced amount of tactile sexual function and protecting a person from transmission of a sexually transmitted disease and/or an undesired pregnancy during sexual intercourse, comprising: one or more treatment substance and at least one condom. In certain aspects, the kit further comprises: a vibrating apparatus for stimulating tactile sexual function. In some facets, the vibrating apparatus is a wearable ring on a penis that comprises a vibrating devise.

Some embodiments provide a composition described herein as a treatment substance for use as a medicament. Further embodiments provide a composition described herein as a treatment substance for use in treating a sexual dysfunction. Other embodiments provide a composition described herein as a treatment substance for use in treating a reduction of sexual sensation, a reduced ease of orgasm/ejaculation, and/or premature ejaculation. Other embodiments provide a method for treating a sexual dysfunction in a subject, the method comprising administering to a subject in need thereof a therapeutically effective amount of a composition described herein as a treatment substance; wherein the sexual dysfunction includes but is not limited to a reduction of sexual sensation, a reduced ease of orgasm/ejaculation, and/or premature ejaculation. Some embodiments provide use of a composition described herein as a treatment substance for the manufacture of a medicament. Other embodiments provide use of a composition described herein as a treatment substance for the manufacture of a medicament for the treatment of a sexual dysfunction. In certain aspects, the sexual dysfunction comprises a reduction of sexual sensation, a reduced ease of orgasm/ejaculation, and/or premature ejaculation. Specific embodiments provide use of a composition described herein as a treatment substance for the manufacture of a medicament for the treatment of a sexual dysfunction; wherein the sexual dysfunction includes but is not limited to a reduction of sexual sensation, a reduced ease of orgasm/ejaculation, and/or premature ejaculation. Other embodiments provide a pharmaceutical composition for the treatment of a sexual dysfunction, comprising a treatment substance described herein. Additional embodiments provide a pharmaceutical composition for the treatment of a sexual dysfunction, comprising a treatment substance described herein; wherein the sexual dysfunction includes but is not limited to a reduction of sexual sensation, a reduced ease of orgasm/ejaculation, and/or premature ejaculation. Further embodiments provide an anti-sexual dysfunction agent comprising a composition described herein as a treatment substance. Some embodiments provide an anti-sexual dysfunction agent comprising a composition described herein as a treatment substance; wherein the sexual dysfunction includes but is not limited to a reduction of sexual sensation, a reduced ease of orgasm/ejaculation, and/or premature ejaculation.

### BRIEF DESCRIPTION OF THE DRAWINGS

FIG. 1 depicts the general location of various transporter proteins on blood-brain barrier endothelial cells, exemplary chemicals that the transporter proteins move into and/or out of the brain, and the general direction of the chemicals' movement. Depicted are enzymatic reactions related to the movement of glutamate (“Glu”) from the brain and conversion of Glu into glutathione (“GSH”) that is transported into the blood for additional enzymatic reactions.

FIG. 2 depicts the general location of various transporter proteins on blood-brain barrier endothelial cells, exemplary chemicals that the transporter proteins move into and/or out of the brain, and the general direction of the chemicals' movement.

FIG. 3 depicts the metabolism of monoamines including serotonin, dopamine, noradrenaline, adrenaline, histamine, and melatonin.

FIG. 4 depicts the general location of various transporter proteins, enzymes, and receptors involved in serotonin production, degradation, release, uptake, and/or receptor binding for neurons and astrocytes. Depicted is the general direction of chemicals' movement, with serotonin depicted as a triangle when functioning as a neurotransmitter.

FIG. 5 depicts the general location of various transporter proteins, enzymes, and receptors involved in serotonin production, degradation, release, uptake, and/or receptor binding for neurons and astrocytes. Depicted is the general direction of chemicals' movement, with serotonin depicted as a triangle when functioning as a neurotransmitter, and the effects of an SERT inhibitor treatment substance.

FIG. 6 depicts the general location of various transporter proteins, enzymes, and receptors involved in dopamine production, degradation, release, uptake, and/or receptor binding for neurons and astrocytes. Depicted is the general direction of chemicals' movement, with dopamine depicted as a triangle when functioning as a neurotransmitter.

FIG. 7 depicts the general location of various transporter proteins, enzymes, and receptors involved in dopamine, noradrenaline, and adrenaline production, degradation, release, uptake, and/or receptor binding for neurons and astrocytes. Depicted is the general direction of chemicals' movement, with dopamine depicted as a triangle, noradrenaline depicted as a trapezoid, and adrenaline depicted as a pentagon when functioning as a neurotransmitter.

FIG. 8 depicts the general location of various transporter proteins, enzymes, and receptors involved in histamine production, degradation, release, uptake, and/or receptor binding for neurons and astrocytes. Depicted is the general direction of chemicals' movement, with histamine depicted as a triangle when functioning as a neurotransmitter.

FIG. 9 depicts the general location of various transporter proteins, enzymes, and receptors involved in Glu, D-serine (“D-Ser”), and glycine (“Gly”) production, degradation, release, uptake, and/or receptor binding for neurons and astrocytes. Depicted is the general direction of chemicals' movement, with Glu depicted as a square, D-Ser as a circle, and Gly as a half-circle when functioning as neurotransmitters.

FIG. 10 depicts the enzymes involved in polyamine (e.g., spermine, spermidine, putrescine, agmatine), nitric oxide, gamma-aminobutyric acid (“GABA”), and other chemicals' production and/or degradation. Arginine decarboxylase and agmatinase reactions (dotted arrows) are not as common (and likely does not occur) in mammals as other reactions in the central nervous system (solid line arrows). Diamine oxidase reactions (dashed arrows) more common in mammals' peripheral tissue than in the central nervous system.

FIG. 11 depicts the general location of various transporter proteins, enzymes, and receptors involved in Glu, D-Ser, Gly, and GABA production, degradation, release, uptake, and/or receptor binding for neurons and astrocytes. GABA production depicted in an astrocyte regarding polyamine metabolism is shown in detail in FIG. 10. Depicted is the general direction of chemicals' movement, with Glu depicted as a square, D-Ser as a circle, Gly as a half-circle, and GABA as a triangle when functioning as neurotransmitters.

FIG. 12 depicts the general location of various transporter proteins, enzymes, and receptors involved in GSH, D-Ser, Gly, and Glu production, degradation, release, uptake, and/or receptor binding for neurons and astrocytes. Depicted is the general direction of chemicals' movement, with Glu depicted as a square, D-Ser as a circle, and Gly as a half-circle when functioning as neurotransmitters.

FIG. 13 depicts the enzymes involved in neurosteroid production and/or degradation, including neurosteroids that are positive allosteric modulators [e.g., androstanediol, allotetrahydrodeoxycorticosterone, allopregnanolone (“APL”)] of the GABAA receptor (“GABAA-R”).

FIG. 14 depicts the general location of various transporter proteins, enzymes, and receptors involved in GABA, Glu, neurosteroid (e.g., APL), and polyamine production, degradation, release, uptake, and/or receptor binding for neurons and astrocytes. Neurosteroid and polyamine metabolism depicted in an astrocyte are shown in detail in FIG. 13 and FIG. 10, respectively. Depicted is the general direction of chemicals' movement, with GABA depicted as a triangle, Glu depicted as a square, and Gly as a half-circle when functioning as neurotransmitters. An agonist for the TRPV1 receptor (“TRPV1-R”) is depicted as a hexagon.

FIG. 15 depicts the general location of various transporter proteins, enzymes, and receptors involved in GABA, Glu, neurosteroid (e.g., APL), and polyamine production, degradation, release, uptake, and/or receptor binding for neurons and astrocytes. Neurosteroid and polyamine metabolism depicted in an astrocyte are shown in detail in FIG. 13 and FIG. 10, respectively. Depicted is the general direction of chemicals' movement, with the enzymatic direction of mitochondrial branched-chain amino transferase (“BCATaseM”) and cytosolic branched-chain amino transferase (“BCATaseC”) reversed relative to FIG. 14, and the location of BCATaseM located in BBB endothelial cells based on human data rather than in astrocytes based on data generally from rats/mice. GABA is depicted as a triangle, Glu depicted as a square, and Gly as a half-circle when functioning as neurotransmitters. An agonist for the TRPV1 receptor (“TRPV1-R”) is depicted as a hexagon.

FIG. 16 depicts the general location of various transporter proteins, enzymes, and receptors involved in acetylcholine production, degradation, release, uptake, and/or receptor binding for neurons and astrocytes. Acetylcholine is depicted as a triangle when functioning as a neurotransmitter.

FIG. 17 depicts the general location of various transporter proteins, enzymes, and receptors involved in acetylcholine production, degradation, release, uptake, and/or receptor binding for neurons and astrocytes. Acetylcholine is depicted as a triangle when functioning as a neurotransmitter, and the effects of an acetylcholinesterase inhibitor treatment substance (“ITS”) in increasing extracellular (e.g., synaptic cleft) acetylcholine amount is shown.

FIG. 18 depicts the general location of various transporter proteins, enzymes, and receptors involved in purine neurotransmitter (e.g., ATP, ADP, adenosine) production, degradation, release, uptake, and/or receptor binding for neurons and astrocytes. Ketogenic diet metabolic pathways for acetoacetate (“ACT”), beta-hydroxybutyrate (“3-HIB”) [e.g., from degradation of 8-carbon octanoic acid (“C8,” “caprylic acid”)/10 carbon decanoic acid (“C10,” “capric acid”) fatty acids] are depicted. Glucogenic diet metabolic pathways for glucose and pyruvate (“PYR”) as also depicted. ATP is depicted as a triangle, ADP is depicted as a pentagon, adenosine is depicted as a circle, and other neurotransmitters depicted as a square, when functioning as a neurotransmitter.

FIG. 19 depicts the enzymes involved in endocannabinoid (“cannabinoid”) production and/or degradation, including neurosteroids that are neurotransmitters [e.g., 2-arachidonoylglycerol (“2-AG”), anandamide].

FIG. 20 depicts the general location of various transporter proteins, enzymes, and receptors involved in cannabinoid neurotransmitter [e.g., 2-arachidonoylglycerol (“2-AG”), anandamide] production, degradation, release, uptake, and/or receptor binding for neurons and astrocytes. In FIG. 20, 2-AG is depicted as a ¾ths of a circle, anandamide is depicted as a circle, Glu is depicted as a triangle, D-Ser is depicted as a half-circle, GABA is depicted as a hexagon, and acetylcholine is depicted as a square, when functioning as a neurotransmitter.

FIG. 21 depicts the relative intensity of tactile sexual sensation and the value (“score”) assigned for TSF for A and B measurements based on tactile sexual sensation intensity. A score of 0.0 is no sexual sensation, while a score above 0.0 and below 8.0 is a not pleasurable sexual sensation, and a score from 8.0 to below 9.0 is weakly pleasurable (“pleasant”). A TSF score above 9.0 is pleasurable.

FIG. 22 depicts the relative intensity of TSF for C score in the plateau phase, the A score right before the first muscle contraction of ejaculation during the orgasm phase and B score immediately after the first muscle contraction of ejaculation at the beginning of the resolution phase of the male sexual response cycle for: a normal sexual response of TSF, TSF when no treatment substances are ingested, and TSF for examples where ingestion of one or more preferred treatment substance(s) that achieved a preferred A/B score of at least 8.0 in combination with a preferred C score of at least 2.0 and/or a more preferred A/B score of at least 9.0 regardless of the C score.

FIG. 23 depicts a proposed model of transporter proteins, enzymes, and receptors involved in schizophrenia. In FIG. 23, Glu is depicted as a square, D-Ser is depicted as a circle, GABA is depicted as a triangle, and dopamine is depicted as a hexagon, when functioning as a neurotransmitter.

### DETAILED DESCRIPTION OF THE EMBODIMENTS

For a further understanding of the nature and function of the embodiments, reference should be made to the following detailed description. Detailed descriptions of the embodiments are provided herein, as well as, the best mode of carrying out and employing the present invention. Various substances such as a prescription drug, a non-prescription drug, an experimental drug, a chemical, a nutritional supplement (e.g., a preparation of a biological material from a plant, a fungi, a microorganism, etc. such as an extract, a powder, etc.) have inhibitory properties and/or activator properties for various enzymes, transporter proteins, receptors, etc. described herein and as would be known to one of ordinary skill in the art, and it is contemplated that these substances may be used as a treatment substance (e.g., alone; or in combination with each other) based on those properties in accordance with the disclosures herein. It should be understood that a treatment substance, a chemical, a compound, a proteinaceous molecule, a method, a procedure, and/or a technique described herein are presently representative of various embodiments. It will be readily appreciated that the embodiments are well adapted to carry out and obtain the ends and features mentioned as well as those inherent therein. It is to be understood, however, that the present invention may be embodied in various forms. Therefore, specific details disclosed herein are not to be interpreted as limiting, but rather as a basis for the claims and as a representative basis for teaching to employ the present invention in virtually any appropriately detailed system, structure or manner. As used herein, other than a composition of matter (e.g., a chemical composition), an “article,” “article of manufacture” or “manufactured article” refers to a product (e.g., a tool) that is made and/or altered by the hand of man that lacks moving parts, and a machine/device/apparatus is the same as an article of manufacture but has moving parts. An example of an article of manufacture is a condom designed to be worn on a man's penis or inserted in a woman's vaginal tract; and typically designed to protecting a person (e.g., both sexual partners) from transmission of a sexually transmitted disease and/or an undesired pregnancy during vaginal sexual intercourse. An example of a machine/device is a vibrating apparatus (e.g., a vibrator) designed to stimulate tactile sexual function, such as, for example, a ring worn by a man on his penis that comprises a vibrator. In some embodiments, a composition (e.g., a “treatment substance”) described herein may be referred to as a product and/or a medicament (e.g., a treatment substance for use as a medicament). It is known to a skilled person of the art of the location and activity of the various cells (e.g., a neuron, a glial cell such as astrocyte, an endothelial cell in a capillary, etc.), proteins (e.g., an enzyme, a receptor, a transporter proteins, etc.), ligands (e.g., an agonist, an antagonist, an allosteric modulator, a substrate for an enzyme, etc.) and other biomolecules and chemicals described herein and depicted in the Figures (“Fig,” “FIG”) as merely the context in which the disclosures herein, particularly in the working examples, are made of the embodiments of the invention of a treatment substance, a method, an article of manufacture, etc. that affects a sexual dysfunction or other condition (e.g., a neurological disorder). Other feature(s) will be readily apparent from the following detailed description; the specific examples and the claim(s); and an adaptation, a change, an equivalent, a modification, a substitution, a deletion, and/or an addition of a material (e.g., a treatment substance), a method, a procedure and/or a protocol and other use(s) and/or modification(s) that may be made to the embodiment(s) disclosed herein without departing from the scope and spirit of the invention or as defined by the scope of the appended claim(s).

All patent(s) and publication(s) mentioned in this specification are herein incorporated by reference to the same extent as if each individual publication was specifically and individually indicated to be incorporated by reference. For the sake of brevity, citation to a reference (“Ref.”) herein may be made based on the reference's citation in a Table, such as, for example, “Ref 24, and 27, Table 5” referring to “24) Scammell, T. E. et al. Sleep. 2019 42(1):zsy183” and “27) Hoffman, G. E. and Koban, M. PLoS One 2016 11(12):e0152252” listed in Table 5. Inside a Table herein, references for that Table are merely cited by number (“Reference No”) wherein the number refers to a citation listed at the bottom of the Table.

Various terms will be used herein as follows. Other than the claims, the terms “a,” “an,” “the,” and “said” before a word, or “(s)” at the end of a word, means one or more than one [e.g., “an enzyme” and “enzyme(s)” means one or more]; and for the sake of brevity, in the Tables a word may be in singular form though it will be understood that the word may refer to more than one. In a claim, the terms “has,” “having,” “have,” “including,” “include” and/or “includes,” has the same meaning as “comprising,” “comprises,” and “comprise”; and in a claim when used in conjunction with the forgoing terms in quotations (e.g., “has,” include,” “comprise,” etc.) the words the terms “a,” “an,” “the,” and “said” before a word or “(s)” at the end of a word means one or more than one. The word “another” before a word means at least a second or more. One or more backward slash symbols “\”, “\\”, “\\\”, etc. are used herein to separate subcategories, particularly in a Table, such as, for example, separating a treatment substance that promotes GABAergic neurotransmission signaling from a treatment substance that promotes an increased amount of cysteine (e.g., “GABA \ cysteine,” “GABA\cysteine”). A forward slash “/” symbol between two words means “and/or” (e.g., “neurological/biological function” or “neurological/biological function” means “neurological and/or biological function”). Various genera and sub-genera described herein are contemplated both as an individual treatment substance, as well as and mixtures and combinations, and may be described in the claims as “at least one selected from,” “a mixture thereof” and/or “a combination thereof,” and such like. The phrase “a combination thereof” “a mixture thereof,” “and/or,” the slash symbol “/” in a listing; the phrase “such as,” followed by a listing; and/or a listing within parentheses “( ),” brackets “[ ],” and/or braces “{ }” refers to any combination/sub-set of listed component(s). For example, the phrases “such as A, B, or C” or “(e.g., A, B, C)” or “A/B/C” refers to various combinations that include, for example, the combination “A” and “B” as well as a combination “A” and “C” and the combination “B” and “C.” Combinations of related species described herein though not directly placed in such a listing are also contemplated. For example, an inhibitor of an enzyme described in the text and another inhibitor of the same enzyme listed in a Table in different sections of the specification may be claimed individually and/or as a combination, as they are part of the same genera of enzyme inhibitor(s). A range described herein includes all integers and sub-ranges comprised within a described range. For example, a range “0.2% to 0.9%” provides specific values within the cited range, such as, for example, 0.2%, 0.3%, 0.4%, 0.5%, 0.6%, 0.7%, 0.8%, and/or 0.9%, as well as various combinations of such specific values, such as, for example, 0.2%, 0.5% and 0.9%; 0.5% and 0.6%; or 0.5% and 0.8%; as well as sub-ranges such as 0.2% to 0.4%; 0.5% to 0.8%; or 0.3% to 0.7%, etc. In another example, a cited range of “5 minutes to 120 minutes include specific values within the cited range, such as, 17 minutes; various combinations of specific values such as 28 minutes and 33 minutes; as well as sub-ranges such as 55 minutes to 65 minutes, etc. The word “about,” including when used in a claim, refers to any rounding of digit(s) and/or measurement error(s) for a value and/or range.

Various terms will be used herein as follows. Abbreviations, acronyms and alternative names (referred to herein as an “AAA”) that are used herein in sentences are typically shown in quotation marks after the named chemical, protein, term, etc. and are often placed in parentheses, brackets, and/or braces such as, for example, nicotinamide adenine dinucleotide (“NAD”) that is metabolically produced from niacin (“Nicotinic acid,” “vitamin B3”); nicotinamide adenine dinucleotide phosphate [“NAD(P),” NADP”]; reduced nicotinamide adenine dinucleotide phosphate [“NADPH”]; reduced and flavin adenine dinucleotide (“FAD”) that is enzymatically created from riboflavin (“vitamin B2”); thiamine pyrophosphate that is enzymatically created from vitamin B1; and flavin mononucleotide (“FMN”). The AAA used herein include: dehydroepiandrosterone (“DHEA”); DHEA sulfate ester (“DHEAS”); brain-derived neurotrophic factor (“BDNF”); allotetrahydrodeoxycorticosterone (“THDOC”); 5alpha-dihydrodeoxycorticosterone (“dihydrodeoxycorticosterone”); 5alpha-dihydroprogesterone (“dihydroprogesterone”); allopregnanolone (“APL”); 3alpha-androstanediol (“androstanediol”); 11-deoxycorticosterone (“deoxycorticosterone”); anandamide (“AEA,” “N-arachidonoyl-ethanolamine”); N-arachidonoyl phosphatidylethanolamine (“NAPE”); alpha-ketoisovalerate (“KIV”); alpha-keto-beta-methylvalerate (“KMV”); alpha-ketoisocaproate (“KIC”); branched-chain keto-acid [“BCKA”; e.g., KIV, KMV, KIC]; 3,4-dihydroxyphenylglycolaldehyde (“DOPEGAL”); 5-hydroxyindoleacetic acid aldehyde (“5-HIAAA”); S-adenosyl-methionine (“SAMe”); S-adenosyl-homocysteine (“SAH”); 3,4-dihydroxyphenylacetic acid (“DOPAC”); 3,4-dihydroxyphenylacetaldehyde (“DOPAL”); 3-methoxy-4-hydroxphenyl-acetaldehyde (“3M4HA”); N-acetyl-5-methoxytryptamine (“Melatonin”); phenibut (“beta-phenyl-GABA”); N-pantoyl-GABA (“Hopantenic acid”); beta-hydroxy-GABA (“GABOB”); rauwolscine (“alpha-yohimbine”); and a mixture of rauwolscine and corynanthine (“yohimbine”). Certain words or terms may lack commonly used subscripts and superscripts (e.g., “H₂O” may be written herein as “H2O”; “Ca²⁺” may be written herein as “Ca2+”). Certain terms that are normally italicized may be in normal font herein, such as the scientific names for plants and animals (e.g., “Rosmarinus officinalis” may be written herein as “Rosmarinus officinalis”). The term “Rx” before a list of chemical(s) in braces [e.g., “Rx{Atomoxetine}”] indicates the chemical typically requires a prescription for use in one or more jurisdictions. Common AAAs for units of measurements are used herein, and include: millimolar (“mM”); micromolar (“μM”); nanomolar (“nM”); centimeter (“cm”); millimeter (“mm”); kilogram (“Kg,” “kg”); gram (“g”); milligram (“mg”); microgram (“μg”); nanograms (“ng”); second (“sec”); minute (“min”); hour (“hr”); etc. A chemical that is a L-stereoisomer may not have the “L-” listed (e.g., “L-serine” may be written herein as “serine”; L-dehydroascorbic acid may be written herein as “dehydroascorbic acid”; L-theanine may be listed as “theanine”) to help differentiate from a D-stereoisomer of the chemical that will have the “D-” listed (e.g., Serine and D-Serine are L- and D-stereoisomers, respectively). Examples of L stereoisomers that may be written without the “L-” include magnesium L-threonate (“magnesium threonate”), L-norvaline (“norvaline”), L-ornithine (“ornithine”), and acetyl-L-carnitine (“acetyl-carnitine”). At typical physiological conditions the ionic form and non-ionic form of many chemicals will readily change from one form to the other form, and the ionic and non-ionic name of a chemical may be used interchangeably [e.g., “aspartate” refers to both aspartate and aspartic acid; “pyroglutamic acid” refers to pyroglutamate and pyroglutamic acid (also known as “5-Oxoproline,” “pGlu,” “2-Pyrrolidone-5-Carboxylate”); the abbreviation “AKG” refers to alpha-ketoglutarate/alpha-ketoglutaric acid; the abbreviation “GHB” refers to gamma-hydroxybutyric acid/gamma-hydroxybutyrate; etc.]. The abbreviations for various amino acids and related chemicals herein are: alanine (“Ala”); beta-alanine (“Beta-Ala”); arginine (“Arg”); aspartic acid/aspartate (“Asp”); asparagine (“Asn”); cysteine (“Cys”); cystine (“Cys2”); glutamic acid/glutamate (“Glu”); glutamine (“Gln”); glycine (“Gly”); histidine (“His”); isoleucine (“Ile”); leucine (“Leu”); lysine (“Lys”); methionine (“Met”); phenylalanine (“Phe”); proline (“Pro”); serine (“Ser”); threonine (“Thr”); tryptophan (“Trp”); tyrosine (“Tyr”); valine (“Val”); hydroxylysine (“Hyl”); hydroxyproline (“Hyp”); ornithine (“Orn”); and sarcosine (“Sar”). A chemical that alters the amount of a particular chemical an animal/organ/tissue/cell type (e.g., a human, the brain, the liver, a presynaptic neuron, an astrocyte) after ingestion may be referred to using the suffix “ic” with the particular chemical. For example, a positive “cysteineic” refers to a chemical that increases the amount of Cys in the body, such as in the blood, in various cells (e.g., endothelial BBB cell, neuron, astrocyte) and/or in the intercellular space (e.g., between astrocytes and neurons) after ingestion, such as, Cys, a Cys prodrug [e.g., N-acetyl-cysteine (“NAC”)] that is converted (e.g., by enzymatic activity/a non-enzymatic chemical reaction) into Cys, a precursor that promotes Cys creation, and such like. In another example, a negative cysteineic herein refers to a chemical that reduces the amount of Cys the brain after ingestion. Other examples of such terms include “GABAic,” “glutamateic,” and “sertoninic,” for a chemical that alters the amount of GABA, glutamate, and serotonin respectively. For the sake of brevity, a substance that is precursor to another substance in the body will be referred to herein as a “precursor” to the another substance. In an example, a positive cysteineic is also a positive GSHic as cysteine is a precursor to GSH. A chemical that has, or may have, a biological activity (e.g., an agonist for a receptor, an enzyme inhibitor, etc.) is referred to herein as an “Active”. Often a treatment substance comprises an Active, and often other chemicals, obtained from a biological source such as, for example, a whole plant dried and turned into a powder; a liquid extract derived from a whole plant; dried plant flower, leaves, stems rather than an extract of a whole plant material; an extract using a different solvent, an extract dried into a powder; a dried extract from a fungus; a liquid extract from a flower, etc. and such like materials are referred to herein as a “preparation” of the respective biological material and/or the Active of interest. For example, the plant Piper methysticum (“Kava”) comprises the Active kavalactone, a genus of various Active chemical species (e.g., desmethoxyyangonin, kavain, dihydrokavain, methysticin, yangonin), and kavalactone genus and/or some of the Active species are, or may be, an agonist for one receptor, an inhibitor of an enzyme, etc. Various Piper methysticum Preparations are known in the art and available from vendors. A Piper methysticum (“Kava”) rhizome with root liquid extract from one vendor and a Piper methysticum root extract powder from another vendor are may both be referred to herein as a “Piper methysticum Preparation,” “Kava preparation,” a “preparation of Kava,” and such like; though to distinguish between the two preparation from each other and other Piper methysticum Preparation(s) known in the art and available from vendors, specific designations of “Kava” and “Kava Liquid” may be given to the two specific preparations, particularly in the working examples. A preparation notable for comprising one or few Active(s) may be referred to by the Active(s) of interest. For example, “Resveratol” refers to a composition that comprises the Active trans-resveratol, and possibly further comprises cis-resveratrol. A specific Polygonum cuspidatum Preparation from a vendor comprising an Active of interest trans-resveratrol may be referred to herein as “Trans-Resveratrol” to distinguish the specific preparation used in the working examples from other Polygonum cuspidatum Preparation(s)/Resveratol Preparation(s) known in the art.

In some embodiments, a composition described herein, such as one or more treatment substance(s) (e.g., a treatment substance comprising an Active), may consist essentially of or consist of the treatment substance(s); however, in any of the compositions described herein in terms of consisting essentially of or consisting of the defined treatment substance(s)/Active(s) do not exclude the composition also comprising one or more of non-active ingredient(s)/component(s) that do not significantly/substantially influence a sexual dysfunction/sexual function or other condition, such as a solvent(s), diluent(s), excipient(s), filler(s), binder(s), preservative(s), flavoring(s), and the like. For example, a treatment substance may be formulated (e.g., from a vendor) as a powder comprising an excipient such as silicon dioxide, and the powder being contained within a capsule comprising hydroxypropyl methylcellulose (“hypromellose”). In another example, one or more treatment substance(s) described herein may be formulated in a container (e.g., a bottle, a flask, etc.) such as treatment substance(s) dissolved in water [e.g., water comprising non-active flavoring(s)] for ease of ingestion/rapid absorption of the treatment substance(s). Such non-active ingredient(s)/component(s) (e.g., silicon dioxide, hydroxypropyl methylcellulose, water, non-active flavoring) are typically not listed herein for the sake of brevity, and such non-active ingredients/component(s) may be included in a composition comprising, consisting essentially of, or consisting of one or more treatment substance(s) (e.g., a composition comprising an Active) described herein and in the Claims.

Nested parentheses “( ),” brackets “[ ],” and/or braces “{ }” may be used herein such as, in a Table, as appropriate, for the sake of brevity to state information. For example, as used herein for the sake of brevity, particularly in the Tables, the details of a treatment substance's source (e.g., a preparation from a plant), and additional information (“Info”) such as details of the Active(s), the Active(s) known mechanism(s) of action/or possible mechanism(s) of action contributing to the treatment substance's activity, may be briefly stated such as, for example, in Table 2 for under the listed transporter protein BCRP the statement “ITS: . . . Trifolium pratense Preparation [Active: Biochanin A, Formononetin]” indicates a Trifolium pratense Preparation comprising the Active(s) Biochanin A and/or Formononetin, is known to/or associated with an inhibitory activity (“ITS”) of BCRP, and that another composition comprising the Active(s) Biochanin A and/or Formononetin is contemplated has having a BCRP inhibitory activity as well. In another example, at Table 2 under “MRP2” the listing “ATS: Hypericum perforatum Preparation [Active: Hyperforin, Hypericin (Hypericin dose above 1 mg/human/day increased MRP2 amount)]” indicates that a Hypericum perforatum Preparation comprising the Active(s) Hyperforin and/or Hypericin is known to/or associated with an activation activity (“ATS”) of MRP2, and that the Active hypericin increased MRP2 protein amount at the specified dose of “1 mg/human/day” in a human, and that it is contemplated that another composition comprising the Active hypericin will also have a similar activity particularly at the specified dose per day. In another example, at Table 4, under NMDA-R, the statement “ITS: Inhibitor: . . . Panax genus Preparation {e.g., A Panax ginseng/notoginseng/quinquefolius Preparation [Active: Ginsenoside (e.g., Rb1/Rb2/Rc/Re/Rf/Rg1/Rg2/Rg3/Rh1/Rh2; Inhibits NMDA-R's increase in Ca2+ in neurons)]} indicates that the NMDA receptor is inhibited by a Preparation from the Panax genus such as a Preparation from the Panax species Panax ginseng, Panax notoginseng, and/or Panax quinquefolius particularly wherein the Preparation comprises the genus of Active(s) ginsenoside, as ginsenoside is know to/or associated with the NMDA receptor inhibition activity, and in particular the species of Active(s) known in the art as “Rb1, Rb2, Rc, Re, Rf, Rg1, Rg2, Rg3, Rh1, and/or Rh2” is known to/or associated with an inhibitory activity for the NMDA receptor, and that the inhibitory activity of the Active(s) is known to/or are associated with inhibiting an increase in Ca2+ in a neuron upon activation of a neuron's NMDA receptor/inhibiting activation of a neuron's NMDA receptor so that the increase in Ca2+ is reduced/does not occur, and that it is contemplated that another composition having such Active(s) will have like activity in inhibiting the activity of a NMDA receptor. In another example, at Table 4, under the “GABAA-R” an “ITS: Antagonist: . . . Apigenin [Affects GABAA-R Having Alpha1/Beta1/Gamma2S Subunit]; Matricaria chamomilla Preparation [Active: Apigenin]” is listed wherein apigenin is known as a GABAA receptor antagonist, particularly for a GABAA receptor comprising an Alpha1, a Beta1, and/or a Gamma2S subunit(s), and that a preparation from Matricaria chamomilla comprising the Active apigenin has like activity. In a further example, at Table 17A a statement of “Withania somnifera (‘Ashwagandha’) [A Withania somnifera Preparation; (An ‘Ashwagandha Preparation’)” indicates that a preparation from Withania somnifera is referred to herein as (and often in the art) as Ashwagandha; and at Table 45A, at the listing for “Ashwagandha” under “Classification(s) [Mechanism of Action]” it is stated “Positive GABAergic [Active: Withanolide (e.g., Withaferin A) (GABAA-R Agonist/PAM); Active: Withanolide (e.g., Withaferin A)/Triethylene Glycol (GABAARho-R Agonist/PAM)]” which, for the sake of brevity as would be understood by those of skill in the art in light of the disclosures herein, indicates that a preparation of Withania somnifera (also commonly known as Ashwagandha) has positive GABAergic activity, that the genus of Active(s) withanolide, particularly the species of Active(s) withaferin A is known to have/or is associated with GABAA receptor Agonist and/or PAM activity, and that the Active triethylene glycol and/or the genus of Active withanolide, particularly the species of Active withaferin A, is known to have/or is associated with GABAARho-R Agonist and/or PAM activity and that it is contemplated that a treatment substance comprising such Active(s) would have like activity(s).

A ketone body includes acetoacetate (“ACT”), beta-hydroxybutyrate (“3-HIB”), and acetone. Glutathione (“GSH”) refers to reduced glutathione, and glutathione disulfide (“GSSG”) refers to oxidized glutathione. A nucleoside includes a purine nucleoside (e.g., adenosine, guanosine, inosine) and a pyrimidine nucleoside (e.g., thymidine, cytidine, uridine). A nucleobase includes adenine, guanine, hypoxanthine, thymine, cytosine, and uracil. A monoamine includes noradrenaline (“norepinephrine”), adrenaline (“epinephrine”), dopamine, serotonin (“5-hydroxytryptamine,” “5HT”), histamine, and melatonin. Additional abbreviations and acronyms used herein include oxidation (“OX”), central nervous system (“CNS”), and mitochondria (“MIT”). Various receptor proteins are typically referred to by acronyms, abbreviations, and other terms for the sake of brevity, and as used herein, a receptor protein for a ligand, may forgo use of the word “receptor” and instead use “-R” to indicate the term refers to a receptor protein (e.g., a GABA receptor may be referred to as “GABA-R”). For the sake of brevity, multiple receptors herein (e.g., a Figure) may be referred to abbreviated using parenthesis ( ) and the and/or symbol “/”, such as, for example, the receptors 5HT4-R, 5HT6-R, and/or 5HT7-R referred to as, for example, “5HT(4/6/7)-R” or “5HT4/6/7-R.”

As used herein: A proteinaceous molecule comprises a polymer formed from two or more amino acids, which may be the same or different amino acids. Examples of a proteinaceous molecule includes a peptide between 3 to 100 amino acids in length, a polypeptide of 101 or more amino acids (e.g., 100,000 amino acids) in length, and/or a protein comprising three amino acids or greater in length that matches the length and sequence of a biologically produced proteinaceous molecule encoded by the genome of an organism.

Often models of human neurological/biological function are based on non-human animal experiments as described in the art. Experiments on human components of neurological/biological function (e.g., a neurotransmitter, a neurotransmitter receptor, a transporter protein, an enzyme, a biological cell, etc.) described in the art have shown similarities or differences with non-human animal components of neurological/biological function. As used herein, many sentences will begin with “For humans,” “For mice,” etc. to clarify the biological source of the component of neurological/biological function described in the art. The term “For mammals” refers to a combination of rats, mice, and/or human biological sources, and possibly others (e.g., Guinea pigs, cows, pigs), that were used to characterize the component of neurological/biological function. For brevity, sentences that lack such a clarifying term refer to the nearest preceding sentence having such a term in the same paragraph, and in the absence of such a clarifying term the biological source is “For mammals.” A component of neurological/biological function is often referred by the designation of the human version of the the component regardless of the component species of origin. For example, human excitatory amino acid transporter (“EAAT”) proteins have rodent counterparts, such as human EAAT1 having a rodent counterpart referred to in the art as GLAST; and a sentence beginning “For rats” may refer to the human name, EAAT1, and not GLAST for the protein in the body of the sentence to make comparisons from other species to humans more readily understood. In some instances, the Human Genome Organization gene name may be associated with a protein's name to differentiate proteins' that are similarly named. The biological source of the components of neurological/biological function in the Tables and Figures (“Fig,” “FIG”) herein, unless otherwise specified, are “For mammals,” and are provided for ease of reference to the interactions of the treatment substances described herein with these components of neurological/biological function in increasing, decreasing, and/or otherwise modifying neurotransmitter signaling and/or other biological function(s).

As used herein, a ligand is a chemical that contacts (“binds”) a protein, at a particular region of the protein (“binding site”). As used herein, a “main” ligand refers to the ligand that binds to a binding site that is dominant (“main binding site”) in initiating the primary function of the protein. For example, an enzyme is a protein that, upon binding of a main ligand (“substrate”) to the enzyme's main binding site (“active site,” “catalytic site”), accelerates (“catalyzes”) a chemical reaction wherein the substrate is converted into a different chemical (“product”) that is released by the enzyme. As would be known to one of ordinary skill in the art, in the term “substrate” is also a ligand moved by a transporter protein, and the context of whether a transporter protein or enzyme is acting on a substance referred to herein as a substrate should be applied as appropriate, though a transporter protein's substrate will be preferentially referred to herein as a “substance” moved/transported for ease of distinction. In another example, a main ligand is substance moved by transporter protein, or an endogenous agonist that promotes the activity of a receptor upon binding the receptor's main binding site. In another example, a ligand can bind and be readily released from an enzyme's active site is a “competitive inhibitor” to the substrate for the enzyme, while a ligand that binds the enzyme's active site with an extremely slow-release rate (e.g., not detectably released) relative to the substrate's release rate is a “non-competitive inhibitor.” Similarly, a ligand that binds a transporter protein and is readily moved by the transporter protein is also a competitive inhibitor to the substance for the transporter protein, while a ligand that binds the transporter protein and is moved with an extremely slow movement rate relative to the substance's movement rate is a non-competitive inhibitor. Affinity is the ability of a ligand to bind a protein, and alteration of the activity of the activity of the protein by the ligand's binding is referred to as the “efficacy” of the ligand. For example, an agonist is a ligand having high efficacy (e.g., 100% efficacy) to promote the activity of a receptor (e.g., activation of a metabolic pathway, opening of a channel to allow movement of an ion) upon binding the receptor' main binding site. Often a receptor type is named after an agonist (e.g., an AAA for the agonist), such as an N-methyl-D-aspartate (“NMDA”) receptor (“NMDA-R”); an alpha-amino-3-hydroxy-5-methyl-4-isoxazoleproprionic acid (“AMPA”) receptor (“AMPA-R”), a prostaglandin E receptor 1 (“EP-1R”), etc. An agonist having 100% efficacy is a known as a full agonist, while an agonist having less efficacy up to 1% is a partial agonist, though as used herein a full agonist or a partial agonist may be referred to as an “agonist.” An antagonist is a ligand that does not activate a receptor's activity upon binding of the agonist's binding site, and has about 0% efficacy, and reduces the ability of an agonist to bind the receptor while the antagonist is binding the receptor's main binding site. A competitive (“reversible”) antagonist can bind and be readily released from a receptor's main binding site, while a non-competitive (“irreversible”) antagonist has an extremely slow-release rate relative to an agonist after binding the receptor's main binding site. A ligand may have multiple activities, as for example, as a partial agonist may also function as an antagonist by preventing a full agonist from binding the receptor while the partial agonist binds the main binding site. An inverse agonist has negative efficacy by reducing the activity of a receptor upon binding the receptor's main binding site. For example, a constitutively active receptor is capable of producing a biological response without a bound ligand, and an inverse agonist binding a constitutively active receptor may reduce the activity (e.g., reduce the activation of a metabolic pathway, close an ion channel) of the receptor. An allosteric modulator is a ligand that binds a protein at a different site (“allosteric site”) than a main binding site for the protein, and upon binding of the allosteric modulator the activity of the protein upon binding of a main ligand is altered. A positive allosteric modulator (“PAM”) increases the protein's activity upon binding a main ligand, and a negative allosteric modulator (“NAM”) decreases the protein's activity upon binding a main ligand. For example, positive allosteric modulator (“PAM”) for a neurotransmitter receptor binding the receptor will increase the activity of the receptor upon binding of an agonist to the receptor relative to a neurotransmitter receptor lacking a PAM binding the receptor. In another example, negative allosteric modulator (“NAM”) for an enzyme decreases the activity of the enzyme to convert a substrate into a product relative to an enzyme lacking a NAM binding the enzyme. In another example, a NAM binding a BBB transporter protein will decrease the rate of movement of a substance typically moved by a BBB transporter protein across the BBB. In some instances, a ligand binding a protein may increase (“upregulate”) or decrease (“downregulate”) the amount of a protein in the location that the protein functions. For example, a ligand may bind protein to promote the protein's activity of reducing the amount of nearby neurotransmitter receptor at the plasma membrane of a cell, and thus the activity of a neurotransmitter agonist for the receptor is reduced by having a lesser amount of the receptor being available for activation by the agonist. An enzyme may incorporate a cofactor, such as a vitamin/vitamin derivative/metal, for optimal enzymatic activity, and such a cofactor may be ingested as a treatment substance. An enzyme may also be classified by an enzyme commission number (“EC”) that refers to an enzyme by the chemical reaction the enzyme catalyzes. For example, protein kinase C (“PKC”) refers to an EC 2.7.11.13 enzyme; though other enzymes described herein [e.g., ecto-alkaline phosphatase (“EAPase”)] may be listed without an EC number. Small molecules such as water, carbon dioxide, hydrogen peroxide, ammonia/ammonium, adenosine triphosphate (“ATP”), adenosine diphosphate (“ADP”), adenosine monophosphate (“AMP”), and phosphate are generally excluded from listed enzymatic reactions as a substrate or product described herein for brevity. Most enzyme catalyzed reactions are reversible, where at physiological conditions the general preference is to convert the substrate(s) (“S”) into product(s) (“P”) wherein a product are more abundant than a substrate. In some cases, such as enzymes sequestered in a specific environment (e.g., a cellular organelle such as mitochondria), the environment may promote more of the reverse reaction to occur to increase the amount of a substrate relative to most physiological environments. A treatment substance that provides an abundance of substrate for an enzymatic reaction promotes creation of more product of the enzymatic reaction. A treatment substance that provides an abundance of a product for an enzymatic reaction promotes the reverse reaction and creation of a substrate. Similarly, a transporter protein described herein as moving a substance predominantly in one direction (e.g., into a cell) under typical physiological conditions may have the transporter protein's activity altered (e.g., reversed in transport direction) due to a treatment substance that increases the amount of the substance (e.g., increases the amount of substance in a cell). As used herein, regardless of the mechanism of action, a ligand (e.g., a treatment substance) that increases the activity of a protein may be referred to herein as an “activator” for that protein, while a ligand that decreases the activity of a protein may be referred to herein as an “inhibitor” for that protein. Treatment substances described herein are often activators (“Activator Treatment Substance,” “ATS”) and/or inhibitors (“Inhibitor Treatment Substance, “ITS”), as some have multiple mechanisms of activity. For example, a treatment substance may be an agonist of a target neurotransmitter described herein. In another example, a treatment substance may both be an agonist of a target neurotransmitter, and be an inhibitor of movement of a substance by a transporter protein. Treatment substances described herein are generally inhibitory or activating of a target component of neurological/biological function (e.g., a transporter protein, an enzyme, a receptor), but may have mechanisms of action that have not been determined. For example, a treatment substance may be inhibitory to a specific transporter protein, including a transporter protein whose relevance to neurological/biological function may be currently unknown, and the inhibitory activity of the treatment substance is currently unknown as the experiment that would determine that mechanism of action has not been conducted and published.

A treatment substance may enter the body by various mechanisms known in the art, such as oral ingestion (e.g., formulations such as powder, tablet, liquid, liposomal, etc.), injection into a blood vessel, a nasal spray/inhalation, and/or eye drops. For example, to improve movement from the gastrointestinal tract into the bloodstream, a treatment substance may be formulated in a liposome and/or other formulation known in the art. A treatment substance described herein often acts in the brain, and a treatment substance enters the brain from capillary blood vessels. The layer of endothelial cells of a brain's capillary vessel with the pericyte cells and the astrocytes adjacent to the endothelial cells and associated proteins (e.g., collagen type IV, fibronectin, heparan sulfate proteoglycan, laminin) act to impede/prevent many chemicals (e.g., a treatment substance) from crossing from the blood into the brain and/or to impede/prevent many chemicals from crossing from the brain into the blood; and these cells and associated proteins are referred to herein as the blood brain barrier (“BBB”). Similarly, protein and cell structures at a tissue located in the brain known as the choroid plexus (“CRP”) will impede/prevent, but often to a lesser degree than the BBB, many chemicals from crossing between the brain and the cerebrospinal fluid (“CSF”), and these cells and associated proteins are referred to herein as the blood cerebrospinal fluid barrier. It is contemplated that in addition to the brain, the spinal cord and peripheral nerve neurotransmitter signaling are affected by many of the treatment substances described herein.

A substance that is hydrophilic and/or has a large molecular size relative to a phospholipid may cross a cell's phospholipid membrane (e.g., a cell's plasma membrane) with the aid of a protein (e.g., a transporter protein, a receptor protein, a channel protein), associated with the cell's phospholipid membrane; and such a phospholipid membrane protein that promotes movement of a substance, regardless of the mechanism of action, are referred to herein as a “transporter” (abbreviated commonly in the Figures as “TP”). For example, a transporter associated with a BBB cell's (e.g., a BBB capillary endothelial cell) phospholipid membrane is known herein as a “BBB transporter,” and a transporter associated with a CRP cell's (e.g., a CRP epithelial cell) phospholipid membrane is known herein as a “CRP transporter.” The location of a BBB transporter on the apical side (“luminal side,” “blood side”) of the plasma membrane is known herein as “Blood-BBB,” the location of a BBB transporter on the abluminal side (“brain side”) of the plasma membrane is known herein as “Brain-BBB.” The location of a CRP transporter on the apical side (“luminal side,” “CSF side”) of the plasma membrane is known herein as “CSF-CRP” and the location of a CRP transporter on the abluminal side (“basal side,” basolateral side,” “blood side”) of the plasma membrane is known herein as “Blood-CRP.” For the sake of brevity, multiple transporters herein (e.g., a Figure) may be referred to abbreviated using the and/or symbol “/”, such as, for example, the transporters MRP1, MRP2, MRP4, and/or MRP5, being referred to as “MRP1/2/4/5” or “MRP(1/2/4/5),” though in some instances space(s) may be included with the “/” symbol such as “Dopamine/Serotonin” for ease of visual review. For mammals, amino acids that are not chemically modified are generally ionized at physiological pH and hydrophilic, and BBB transporters move these amino acids across the BBB. Some amino acids that act as neurotransmitters and/or are readily metabolized into neurotransmitters are often moved (“transported”) out of the brain by BBB transporters. In general, a first chemical that is being moved by an individual transporter (e.g., a BBB transporter) will competitively inhibit a second chemical from being moved at the same time in the same direction. For example, a branched-chain amino acid (“BCAA”) refers to herein to Leu, Ile, and Val; and the different BCAAs competitively inhibit the other BCAAs and other amino acids (e.g., Trp, Tyr, Phe) for crossing the BBB via the LAT1 transporter. In some instances, a chemical may efficiently bind a transporter, but be slowly moved by the transporter relative to other chemicals the transporter moves, and the chemical may be described herein as an inhibitor of the transporter as well as a chemical moved by the transporter (e.g., the SNAT2 transporter moves His, and His is described herein as an inhibitor of SNAT2). Certain transporters will move two or more different chemicals in different (e.g., opposite) directions, are referred to herein as “exchanger(s)” or “antiporter(s)” (e.g., the Xc- antiporter). The presence of one chemical generally will promote the movement of a second chemical by the exchanger. Other transporters move two or more chemicals in the same direction and are referred to herein as “Cotransporter(s)”. When a chemical for an exchanger and/or cotransporter is an atom/ion (e.g., H+, Cl−, etc.) normally present during physiological conditions such an atom/ion is typically not described herein for the sake of brevity. Some chemicals (e.g., putrescine, spermine and spermidine) have a limited ability to cross the BBB regardless of the presence of a transporter for the chemical(s). Lipophilic chemicals that are generally small relative to a phospholipid, such as some drugs and a few neurotransmitters (e.g., phenylethylamine, histamine), often can cross the BBB (e.g., by diffusion) without the aid of a BBB transporter. Some chemicals may move across the BBB by a plurality of mechanisms of movement (e.g., transporter, diffusion, receptor mediated endocytosis). Chemical modification can be used to increase movement of a chemical across the BBB, such as attaching a pivaloyloxymethyl (“pivoxil”), acetyl, vitamin, or other chemical structure to an electrically charged chemical to create a more lipophilic prodrug/precursor of a treatment substance that may more readily move across the BBB (e.g., by diffusion) to more effectively function as a treatment substance in the brain/spinal cord. For example, a lipophilic prodrug that crosses the BBB then being metabolized into a drug that acts as a treatment substance; an amino acid chemically modified to be lipophilic to more readily cross the BBB and then being metabolized into an amino acid that is a precursor of a neurotransmitter that is then metabolized into the neurotransmitter; a neurotransmitter chemically modified to be lipophilic to more readily cross the BBB and then being metabolized into the neurotransmitter, etc. For example, Gln is transported out of the brain with little net transport from the blood into the brain, though a lipophilic prodrug of Gln, N-acetyl-L-glutamine (“AcetylGln”), may diffuse more readily from the blood into the brain and be metabolized into Gln. For example, acetyl-L-carnitine (“acetyl-carnitine”), is more lipophilic than carnitine and crosses the BBB to be metabolized into carnitine in the brain [Ref 3, 51, 108, 229, 230, and 243, Table 2; Ref 96, Table 4; Ref. 5, Table 5; Ref 1, Table 3; Ref 1, Table 8]. In some embodiments, two or more treatment substances may be selected to avoid competitive inhibition (e.g., a treatment substance that uses a transporter and a lipophilic treatment substance that diffuses across cells) in order to allow greater amounts of two or more treatment substances to enter the brain or other tissues. In other embodiments, a competitive inhibitor of a transporter's movement of a treatment substance may be used to evaluate the reduction of the effectiveness of the second treatment substance in improving TSF or another sexual function.

Some physiological conditions and/or chemicals may increase (e.g., starvation, steroids) or decrease (e.g., histamine) the permeability of the BBB and/or a BBB transporter's transport function [Ref. 229, Table 2; Ref. 96, Table 4; Ref 5, Table 5]. The ingestion of a chemical (e.g., a carbohydrate, a protein, a fat, an amino acid, a treatment substance, etc.) may alter (e.g., increase, decrease) the rate of movement of treatment substance(s) across the BBB and/or promote other metabolic effects that could change the effectiveness of the treatment substance(s). For example, carbohydrate (i.e., starches, sucrose) ingestion increases the blood's insulin concentration, and the insulin lowers the blood's BCAA's, Tyr and Phe concentrations by increasing movement of those amino acids into muscles. The lower blood BCAA's concentrations changes the blood's ratio of BCAA to other chemicals (e.g., Trp) that compete for the use of the LAT1 BBB transporter, and thus promotes movement of the Trp into the brain relative to BCAA's, Tyr and Phe. The Trp is metabolized in the brain into an increased amount of the neurotransmitter serotonin. In a further example, for mammals, ingesting protein comprising about 20% BCAAs will result in about 50% of the amino acids released by the liver into the blood being BCAAs. Ingesting protein with little or no carbohydrates promotes increased BCAAs in blood to promote an increased amount of BCAAs and a reduced amount of Trp to enter the brain by the LAT1 transporter, and BCAAs in the brain promote creation of the neurotransmitters Glu and/or GABA. In a further example, Tyr and Phe that are moved from the blood into the brain by the LAT1 transporter are converted into the neurotransmitters dopamine and noradrenaline. Ingestion of protein and carbohydrates does not alter Trp, Tyr and Phe amounts in the blood, but may lower BCAAs' amount in the blood. The amounts of Trp, Tyr, Phe and BCAAs that cross the BBB by the LAT1 transporter into the brain is correspondingly altered so that conversion of Trp, Tyr and Phe into serotonin, dopamine and/or noradrenaline may be increased and conversion of BCAAs' into Glu and/or GABA in the brain may be decreased [Ref. 5, Table 5; Ref. 205 and 228, Table 2]. In another example, Glu is a neurotransmitter transported out of the brain with little net transport from the blood into the brain; and the reduced amount of Glu in the blood reduces the amount of Glu in the brain by promoting movement of Glu from the brain to the blood [Ref. 113, 160, 175, and 224, Table 2; Ref 1, Table 3]. For humans, blood Glu levels are reduced when ingested with carbohydrates relative to ingesting only Glu [Ref. 15, Table 6]. For humans, blood Glu is lowest about 7 AM before eating food [Ref 227, Table 2]. Food and monosodium Glu increase blood glutamate, though food/coffee [i.e., a Coffea arabica Preparation/Coffea robusta Preparation (Active: Caffeine)] does not increase blood Glu. Progesterone and estrogen in blood decreases the blood Glu amount; and insulin/glucagon in the blood reduces the blood Glu amount [Ref 226, Table 2].

Table 1 lists the abbreviation/acronym/alternative name (“AAA”) for various transporters described herein for ease of reference.

TABLE 1
Abbreviation/Acronym/Alternative Name (“AAA”) for Various Transporters

Transporter Type (“AAA”)
AAA Subtype (Gene)
L-Amino Acid Transporter (“LAT”)
LAT1 (SLC7A5); LAT2 (SCL7A8)
Alanine, Serine, Cysteine Transporter (“ASCT”)
ASCT2 (SLC1A5); ASCTI (SLC1A4)
System Alanine, Serine, Cysteine Transporter (“ASC”)
ASC1 (SLC7A10)
Sodium-Coupled Neutral Amino Acid Transporter (“SNAT”)
SNAT1 (SLC38A1); SNAT2 (SLC38A2); SNAT3 (SLC38A3); SNAT5 (SLC38A5);
SNAT7 (SLC38A7); SNAT8 (SLC38A8)
Cationic Amino Acid Transporter (“CAT”)
CAT1 (SCL7A1); CAT2 (SCL7A2); CAT3 (SCL7A3)
Y + L Amino Acid Transporter (“y + LAT”)
y + LAT1 (SCL7A7); y + LAT2 (SCL7A6)
Sodium and Chloride-Dependent Neutral and Basic Amino Acids Transporter
ATB(0, +) (SLC6A14)
Sodium-Dependent Neutral Amino Acids Transporter
BOAT2 (SLC6A15)
Excitatory Amino Acid Transporter (“EAAT”)
EAAT1 (SLC1A3); EAAT2 (SLC1A2); EAAT3 (SLC1A1); EAAT4 (SLC1A6)
Vesicular Glutamate Transporter (VGLUT”)
VGLUT1 (SLC17A7); VGLUT2 (SLC17A6); VGLUT3 (SLC17A8)
Vesicular Excitatory Amino acid Transporter (“VEAT”)
VEAT (SLC17A5)
Cystine-Glutamate Antiporter (“Xc−”)
Xc− (SLC7A11)
GABA Transporter (“GAT”)
GAT1 (SLC6A1); GAT2 (SLC6A13); GAT3 (SLC6A11)
Betaine/GABA Transporter (“BGT”)
BGT1 (SLC6A12)
Glycine Transporter (“GLYT”)
GLYT1 (SLC6A9); GLYT2 (SLC6A5)
Vesicular Inhibitory Amino Acid Transporter (“VGAT”)
VGAT (SLC32A1)
Proline Transporter (“PROT”)
PROT (SLC6A7)
Sodium-Imino Acid Transporter (“SIT”)
SIT1 (SLC6A20)
Neurotransmitter Transporter (“NTT”)
NTT4 (SLC6A17)
Organic Cation Transporter Novel (“OCTN”)
OCTN2 (SLC22A5); OCTN1 (SLC22A4)
Serotonin Transporter (“SERT”)
SERT (SLC6A4)
Noradrenaline Transporter (“NET”)
NET (SLC6A2)
Dopamine Transporter (“DAT”)
DAT (SLC6A3)
High Affinity Choline Transporter (“CHT”)
CHT1 (SLC5A7)
Plasma Membrane Monoamine Transporter (“PMAT”)
PMAT (SLC29A4)
Vesicular Monoamine Transporter (“VMAT”)
VMAT2 (SLC18A2); VMAT1 (SLC18A1)
Organic Anion Transporter (“OAT”)
OAT1 (SLC22A6); OAT3 (SLC22A8); OAT2 (SLC22A7); OAT4 (SLC22A11);
OAT5 (SLC22A10); OAT7 (SLC22A9)
Urate Transporter (“URAT”)
URATI (SLC22A12)
P-Glycoprotein (“P-GP”)
P-GP (ABCB1)
Breast Cancer Resistance Protein (“BCRP”)
BCRP (ABCG2)
Multidrug Resistance Protein (“MRP”)
MRP1 (ABCC1); MRP2 (ABCC2); MRP4 (ABCC4); MRP5 (ABCC5); MRP3 (ABCC3);
MRP6 (ABCC6)
Organic Anion Transporting Polypeptide (“OATP”)
OATP1A2 (SLCO1A2); OATP2B1 (SLCO2B1); OATP3A1V1 (SLCO3A1V1);
OATP3A1V2 (SLCO3A1V2); OATP1A1 (SLCO1A1); OATP1A4 (SLCO1A4);
OATP1A5 (SLCO1A5); OATP1C1 (SLCO1C1); OATP2A1 (SLCO2A1);
OATP4 (SLCO4A1); OATP6 (SLCO6A1)
Organic Cation Transporter (“OCT”)
OCT1 (SLC22A1); OCT2 (SLC22A2); OCT3 (SLC22A3); OCT6 (SLC22A16)
Vesicular Acetylcholine Transporter (“VAChT”)
VAChT (SLC18A3)
Creatine Transporter (“CT”)
CT1 (SLC6A8)
Taurine Transporter (“TAUT”)
TAUT (SLC6A6)
Equilibrative Nucleoside Transporter (“ENT”)
ENT1 (SLC29A1); ENT2 (SLC29A2); ENT3 (SLC29A3)
Concentrative Nucleoside Transporter (“CNT”)
CNT2 (SLC28A2); CNT3 (SLC28A3); CNT1 (SLC28A1)
Vesicular Nucleotide Transporter (“VNUT”)
VNUT (SLC17A9)
Vesicular Polyamine Transporter (“VPAT”)
VPAT (SLC18B1)
Peptide Transporter (“PEPT”)
PEPTI (SLC15A1); PEPT2 (SLC15A2)
Peptide-Histidine Transporter (“PHT”)
PHT1 (SLC15A4); PHT2 (SLC15A3)
Monocarboxylate Transporter (“MCT”)
MCT1 (SLC16A1); MCT2 (SLC16A7); MCT3 (SLC16A8); MCT4 (SLC16A3);
MCT5 (SLC16A4); MCT6 (SLC16A5); MCT7 (SLC16A6); MCT8 (SLC16A2);
MCT9 (SLC16A9); MCT11 (SLC16A11); MCT14 (SLC16A9)
Mitochondrial 2-Oxoglutarate-Malate Carrier Protein (“OCG”)
OGC (SLC25A11)
Aspartate-Glutamate Carrier (“AGC”)
AGC1 (SLC25A12)
Glutamate Carrier (“GC”)
GC1 (SLC25A22)
Mammalian Proton Cation Antiporter (“MATE”)
MATE1 (SLC47A1)
Na+-K+-2Cl- Cotransporter (“NKCC”)
NKCC1 (SLC12A2)
K+-Cl- Cotransporter (“KCC”)
KCC2 (SLC12A5); KCC3 (SLC12A6)
Sodium Potassium ATPase (“Na+-K+ ATPase”)
Na+-K+ ATPase
Bestrophin 1 (“BEST1”)
BEST1
Voltage-Gated Ca2+ Channel (“VGCC”)
VGCC
Volume-Regulated Anion Channel (“VRAC”)
VRAC

Examples of characterized transporters on BBB endothelial cells, neurons, glial cells, and other locations, the gene for the transporter, the substance transported by the transporter (“ST”), and substance(s) that inhibit the function of the transporter (“ITS”) and/or activate the function of the transporter (“ATS”) are shown at Table 2.

TABLE 2
Transporters; Substances Transported (“ST”); Transporter Inhibitors
(“ITS); and Transporter Activators (“ATS”)

Transporter: (Gene) [Location]
Substance Moved by Transporter (“ST”): Substance [Details (Info)] [Reference No]
Inhibitor Treatment Substance (“ITS”): Substance [Details (Info)] [Reference No]
Activator Treatment Substance (“ATS”): Substance [Details (Info)] [Reference No]
LAT1: (SLC7A5) [Blood-BBB/Brain-BBB; Blood-CRP/CSF-CRP; Astrocyte; Neuron]
ST: BCAA, Trp, Tyr, Phe, Met, His, Thr, and/or 5HTP Transported and/or Exchanged for Gln;
DOPA, Gabapentin, Baclofen also Transported
LAT2: (SCL7A8) [Astrocyte; Neuron]
ST: BCAA; Ala; Ser; Pro; Cys; DOPA; Gabapentin; Baclofen
ASCT2: (SLC1A5) [Brain-BBB; Neuron; Astrocyte]
ST: Ala, Ser, D-Ser, Thr, BCAA, Gly, Glu, Asp; Met Transported and/or Exchanged for Gln;
D-Ser Exchanged for Gln/Ala
ITS: Cys [1, 21]
ATS: Cys [Also promotes movement of Gln to the extracellular space]; Pyroglutamate [Inside
a cell promotes extracellular amino acid movement into the cell]; Taurine [23, 41, 42, 21, 175]
ASCT1: (SLC1A4) [Astrocyte]
ST: Ala, Ser, Cys, Thr, Gly, Hydroxyproline; D-Ser Exchanged for Ser
ITS: Hydroxyproline [121]
ASC1: (SLC7A10) [Neuron]
ST: Gly, Ala, Ser, D-Ser, D-Ile, D-Ala, Cys, Thr, Leu, Alpha Aminoisobutyric Acid, Beta-Ala;
Intracellular Gly/D-Ser Exchanged for Extracellular D-Ile
ITS: Hydroxyproline [107]
ATS: Extracellular D-Ile [Promotes D-Ser/Gly Release From Cell] [107, 119]
SNAT1: (SLC38A1) [GABAergic Neuron]
ST: Gln; Ala; Asn; Cys; His; Ser; D-Ser; Met; Gly
ITS: His [11, 12]
SNAT2: (SLC38A2) [Brain-BBB; Glutamatergic Neuron; Glial Cell; Astrocyte]
ST: Ala; Asn; Cys; Gln; Gly; His; Met; Pro; Ser; D-Ser
ITS: His [11, 12]
SNAT3: (SLC38A3) [Blood-BBB/Brain-BBB; Glial Cell; Astrocyte]
ST: Gln; His; Asn; Ser; Ala
ITS: His [11, 12]
SNAT5: (SLC38A5) [Brain-BBB; Astrocyte]
ST: Gln; Asn; His; Ser; Ala
ITS: His [11, 12]
SNAT7: (SLC38A7) [Neuron]
ST: Gln; His; Ser; Ala; Asn; Asp; Glu; Met; Leu; Gly; Arg
ITS: Asn; Asp [68]
SNAT8: (SLC38A8) [Neuron]
ST: Gln; Ala; Arg; His; Asp; Glu; Leu; Pro
CAT1: (SCL7A1) [Blood-BBB/Brain-BBB]
ST: Lys; Arg; Orn; Cys; His
CAT2: (SCL7A2) [Blood-BBB/Brain-BBB]
ST: Lys; Arg; Orn
CAT3: (SCL7A3) [Blood-BBB/Brain-BBB]
ST: Lys; Arg; Orn
y + LAT1: (SCL7A7) [Blood-BBB/Brain-BBB; Neuron]
ST: Arg, Lys, and/or Orn Exchanged for Gln
y + LAT2: (SCL7A6) [Blood-BBB/Brain-BBB; Astrocyte; Neuron]
ST: Arg, Lys, and/or Orn Exchanged for Gln
ATS: Ammonia [Increases y + LAT2 amount] [20]
ATB(0, +): (SLC6A14) [Blood-BBB; Astrocyte]
ST: Ala; Arg; Asn; Cys; Gln; Gly; His; Ile; Leu; Lys; Met; Phe; Pro; Ser; Thr; Trp; Tyr; Val;
D-Ser; D-Ala; D-Met; D-Leu; D-Asp; D-Trp; Beta-Ala; Carnitine; Acetyl-Carnitine;
Propionyl-Carnitine
ITS: Acetyl-Carnitine [40]
BOAT2: (SLC6A15) [Neuron; Astrocyte]
ST: BCAA; Met; Pro
EAAT1: (SLC1A3) [Brain-BBB; Glial Cell; Astrocyte; Bergmann Cell; Oligodendrocyte]
ST: Glu; Asp; D-Asp
ITS: Arachidonic Acid; Zn2+ [NAM] [13, 14, 144]
ATS: Pyroglutamate [Inside a Cell increases extracellular Glu movement into the Cell];
Harmine/D-Asp [Increases Transporter Amount]; Peganum harmala Preparation [Active:
Harmine]; Taurine; Rx{Riluzole (Activates EAATI to reduce extracellular Glu)} [23, 41, 42, 7,
15, 16, 17, 47, 174, 175]
EAAT2: (SLC1A2) [Brain-BBB; Glial Cell; Astrocyte; Oligodendrocyte; Neuron]
ST: Glu; Asp; D-Asp; Cys
ITS: Zn2+ [NAM] [144]
ATS: Arachidonic Acid; Pyroglutamate [Inside a Cell increases extracellular Glu movement
into the Cell]; Harmine/DHEA [Increases Transporter Amount]; Peganum harmala Preparation
[Active: Harmine]; Taurine [13, 14, 23, 41, 42, 7, 15, 16, 17, 18, 175]
EAAT3: (SLC1A1) [Brain-BBB; Neuron; Glial Cell; Astrocyte; Oligodendrocyte]
ST: Cys; Glu; Asp; D-Asp
ITS: Caffeine [Decreases Transporter Amount]; Coffea arabica Preparation/Coffea robusta
Preparation [Active: Caffeine] [19]
ATS: Pyroglutamate [Inside a Cell increases extracellular Glu movement into the Cell];
Taurine [13, 14, 23, 41, 42, 175]
EAAT4: (SLC1A6) [GABAergic Purkinje Neuron; Astrocyte]
ST: Glu; Asp; D-Asp; Alpha-Aminoadipate
ITS: Zn2+ [NAM] [144]
ATS: Taurine [175]
VGLUT1: (SLC17A7) [Glutamatergic Neuron; Astrocyte; Vesicle]
ST: Glu
ITS: Acetoacetate; 4-Methylene-Glutamate; Kynurenic Acid; Xanthurenic Acid [82, 110]
VGLUT2: (SLC17A6) [Glutamatergic Neuron; Astrocyte; Vesicle]
ST: Glu
ITS: Acetoacetate; Glyoxylate; Pyruvate; Phenyl Pyruvate; Alpha-Keto-Beta-Methyl-Valeric
Acid; Beta-Hydroxybutyrate; 4-Methylene-Glu; Kynurenic Acid; Xanthurenic Acid [82, 110]
VGLUT3: (SLC17A8) [Neuron; Astrocyte; Vesicle]
ST: Glu
ITS: Acetoacetate; 4-Methylene-Glutamate; Kynurenic Acid; Xanthurenic Acid [82, 110]
VEAT: (SLC17A5) [Neuron]
ST: Glu; Asp; D-Asp; N-Acetylaspartylglutamate; Sialic Acid
ITS: Acetoacetate [82]
Xc -: (SLC7A11) [Blood-BBB; Astrocyte]
ST: Cystine (“Cys2”) and Glu Exchanged
ITS: Alpha-Aminoadipate; Extracellular Lactic Acid [2, 171]
ATS: Glu [Increased Glu inside a cell such as by another transporter (e.g., EAAT) movement
of extracellular Glu/Asp/Gln into the cell where Asp/Gln are also converted to Glu] [2]
GAT1: (SLC6A1) [GABAergic Neuron; Glial Cell; Astrocyte]
ST: GABA; Betaine; Beta-Ala; 2Na + , Cl-
ITS: Genistein; Glycine max Preparation [Active: Genistein]; Pueraria mirifica Preparation
[Active: Genistein]; Phloretin; Malus sylvestris/domestica Preparation [Active: Phloretin];
Beta-Ala; Arecaidine; Guvacine; Muscimol; Nipecotic Acid; Gabamide [GABA reuptake
inhibitor]; BDNF/PCK Activator [promote movement of GATI from cell′s surface into the
cell]; Rx{Tiagabine (increases GAT1 amount), Deramciclane, Riluzole} [70, 9, 73, 27, 74, 75,
78, 108, 118]
ATS: Forskolin [74, 75]
GAT2: (SLC6A13) [Blood-CRP; Glial Cell; Astrocyte; Neuron]
ST: GABA; Beta-Ala; Taurine; 2Na + , Cl-
ITS: Beta-Ala; Phloretin; Malus sylvestris/domestica Preparation [Active: Phloretin]; Betaine;
Taurine; Arecaidine; Guvacine; Muscimol; Nipecotic Acid; PKC Activator [Promotes
movement of GAT2 from cell′s surface into the cell]; Cinchona officinalis Preparation [Active:
Quinidine]; Rx{Gabamide (GABA Reuptake Inhibitor), Quinidine, Deramciclane, Riluzole} [9,
72, 73, 27, 48, 108, 118]
GAT3: (SLC6A11) [Glial Cell; Astrocyte; Oligodendrocyte; GABAergic Neuron]
ST: GABA; Beta-Ala; Taurine; 2Na + , Cl-
ITS: Beta-Ala; Phloretin; Malus sylvestris/domestica Preparation [Active: Phloretin]; Taurine;
Arecaidine; Guvacine; Muscimol; Nipecotic acid; PKC Activator [Promotes movement of
GAT3 from cell′s surface into the cell]; Cinchona officinalis Preparation [Active: Quinidine];
Rx{Gabamide (GABA Reuptake Inhibitor), Quinidine, Deramciclane, Riluzole} [9, 73, 27, 48,
108, 118]
BGT1: (SLC6A12) [Brain-BBB; Microglia Cell; Neuron]
ST: Betaine; GABA; Pro
ITS: Betaine; Phloretin; Malus sylvestris/domestica Preparation [Active: Phloretin]; Beta-Ala;
Cinchona officinalis Preparation [Active: Quinidine]; Rx{Gabamide (GABA Reuptake
Inhibitor); Quinidine} [9, 24, 27, 48, 108]
GLYT1: (SLC6A9) [Glial Cell; Glycinergic/Glutamatergic Neuron]
ST: Gly
ITS: Sarcosine; Ala; GABA; Pro; N-Arachidonylglycine; Ethanol; Zn2+ [NAM]; PKC
Activator [Promotes movement of GLYT1 from cell′s surface into the cell]; Rx{Pesampator,
Bitopertin} [9, 48, 108, 118, 144, 145]
GLYT2: (SLC6A5) [Glycinergic/GABAergic Golgi Interneuron]
ST: Gly
ITS: Ala; Beta-Ala; N-Arachidonylglycine; Ethanol; PKC Activator [Promotes movement of
GLYT2 from cell′s surface into the cell]; Rx{Amoxapine, Opiranserin} [48, 108]
SIT1: (SLC6A20.A) [Glial Cell; Astrocyte; Microglia]
ST: Pro; Gly; OH-Proline; Betaine
ITS: Sarcosine; Betaine [9]
VGAT: (SLC32A1) [GABAergic Neuron; Vesicle]
ST: GABA; Gly; Beta-Ala
ITS: Beta-Ala; Butyric acid; Nipecotic acid; Rx{Valinomycin, Nigericin, Vigabatrin} [110]
PROT: (SLC6A7) [Glutamatergic Neuron; Vesicle]
ST: Pro
ITS: Sarcosine; His; Cys [48]
NTT4: (SLC6A17) [Neuron; Vesicle]
ST: Pro; Gly; Leu; Ala; Glu; Gln; Met
OCTN2: (SLC22A5) [Blood-BBB; Brain-BBB; Astrocyte]
ST: Carnitine; Acetyl-Carnitine; Gamma-Butyrobetaine; D-Carnitine; Propionyl-Carnitine;
Prodrug Carnitine Conjugate; Choline; Galantamine; Galantamine Hydrobromide; Lycoris
radiatia Preparation [Active: Galantamine Hydrobromide]; Cinchona officinalis Preparation
[Active: Quinidine]; Rx{Donepezil, Verapamil, Quinidine}
ITS: Galantamine; Galantamine Hydrobromide; Lycoris radiatia Preparation [Active:
Galantamine Hydrobromide]; Carnitine; Corticosterone; Betaine; Aldosterone; Choline;
Cinchona officinalis Preparation [Active: Quinidine]; Rx{Donepezil, Cimetidine, Verapamil,
Quinidine} [37, 38, 52]
ATS: Sesquiterpene [e.g., Cynaropicrin (Increases Transporter Amount)] [43]
SERT: (SLC6A4) [Brain-BBB/Blood-BBB; Serotoninergic Neuron]
ST: Serotonin
ITS: Lobeline; Lobelia inflata Preparation [Active: Lobeline]; PKC Activator [Promotes
movement of SERT from plasma membrane into the cell in the absence of ligands binding
SERT]; Rx{Dextromethorphan, Diphenhydramine, Citalopram, Chlorpheniramine,
Escitalopram, Fluoxetine, Fluvoxamine, Paroxetine, Sertraline, Desvenlafaxine, Imipramine,
Duloxetine} [9, 118, 176, 177, 178]
NET: (SLC6A2) [Brain-BBB; Noradrenergic Neuron]
ST: Noradrenaline; Dopamine
ITS: Ginkgo biloba Preparation [Active: Glycoside, Ginkgolide A]; Phenylpiracetam; PKC
Activator/Muscarinic Acetylcholine-R Activator [Promote movement of NET from plasma
membrane into the cell]; Rx{Dextromethorphan, Atomoxetine, Reboxetine, Nomifensine,
Desipramine, Methylphenidate, Viloxazine} [9, 118, 232, 233]
DAT: (SLC6A3) [Dopaminergic Neuron]
ST: Dopamine; Noradrenaline
ITS: Octopamine; Phenylethylamine; Zn2+ [low concentration]; Scutellaria genus Preparation
[e.g., Scutellaria baicalensis/lateriflora/galericulata Preparation; Active: Oroxylin A];
Oroxylum indicum Preparation [Active: Oroxylin A]; Lobeline; Lobelia inflata Preparation
[Active: Lobeline]; Chaenomeles speciosa Preparation; Phenylpiracetam [Stronger Inhibitor for
DAT than NET]; Activator of PKC [Promotes movement of DAT from plasma membrane into
the cell]; Rx{Bupropion, Modafinil, Nomifensine, Dexmethylphenidate, Methylphenidate,
Benzatropine, Etybenzatropine, Armodafinil, Ketamine, Sertraline} [9, 14, 118, 144, 176, 177,
178, 239, 240]
ATS: Zinc/Zn2+ [high concentrations]; Luteolin; Arachis hypogaea Preparation [Active:
Luteolin] [144]
PMAT: (SLC29A4) [CSF-CRP; Neuron; Astrocyte]
ST: Dopamine; Serotonin; Noradrenaline; Adrenaline; Acetylcholine; Adenosine; Histamine
ITS: Acetylcholine; Adenosine; Histamine; Phenylethylamine; Harmaline; Harmine;
Harmalan; Norharmanium; Peganum harmala Preparation [Active: Harmine, Harmaline,
Harmine, Harmalan, Norharmanium] [115, 140]
VMAT2: (SLC18A2) [Dopaminergic / Serotoninergic / Adrenalinergic / Noradrenalinergic /
Histaminergic Neuron; Vesicle]
ST: Histamine; Adrenaline; Noradrenaline; Dopamine; Serotonin
ITS: Phenylethylamine; Lobeline; Lobelia inflata Preparation [Active: Lobeline]; Bietaserpine;
Rauwolfia serpentina Preparation [Active: Reserpine]; Rx{Reserpine, Deserpidine, Ketanserin,
Tetrabenazine, Amiodarone, Deutetrabenazine, Valbenazine} [96, 97, 98, 176, 177, 178, 234]
VMAT1: (SLC18A1)
[Dopaminergic/Serotoninergic/Adrenalinergic/Noradrenalinergic/Histaminergic Neuron]
ST: Adrenaline; Noradrenaline; Dopamine; Serotonin; Histamine
ITS: Phenylethylamine; Rauwolfia serpentina Preparation [Active: Reserpine]; Rx{Reserpine;
Ketanserin} [96, 97, 98]
OAT1: (SLC22A6) [CSF CLP; Neuron]
ST: DOPA; 5HTP; Dopamine; Serotonin; 3-Hydroxyisobutyrate; Beta-Hydroxybutyrate;
3-Hydroxypropionate; Benzoate; 3-Indoxylsulfate; 4-Hydroxyphenylpyruvate;
4-Hydroxyphenyllactate; N-Acetylaspartate; 4-Hydroxyphenylacetate;
2-Oxo-3-Methylvalerate; 2-Oxoisocaproate; 3-Hydroxyvalerate; Prostaglandin E2;
Prostaglandin F2Alpha; cAMP; cGMP; Urate; Hypoxanthine; Xanthine; Corticosterone;
DHEAS; Taurine; Morin; Silybin; Silybum marianum Preparation [Active: Silybin];
Intracellular Dicarboxylic Acid [(e.g., AKG) Exchanged for Extracellular Organic Anions];
Rx{Cimetidine, Ranitidine}
ITS: Corticosterone; Rheum genus Preparation [Active: Rhein, Chrysophanol, Physcion];
Punica granatum Preparation [Active: Gallic acid]; Terminalia bellirica Preparation [Active:
Gallic Acid]; Salvia miltiorrhiza Preparation [Active: Lithospermic Acid, Rosmarinic Acid,
Salvionolic Acid A, Salvionolic Acid B, Tanshinol]; Ginkgo biloba Preparation [Active: 17:1
Ginkgolic Acid]; Glycyrrhizae genus Preparation [e.g., Glycyrrhizae uralensis/glabra
Preparation; Active: 18Beta-Glycyrrhetinic Acid; Glycyrrhizin (18Beta-Glycyrrhetinic Acid
Precursor)]; Rx{Probenecid, Ranitidine} [46, 50, 52, 60, 63, 135]
ATS: Punica granatum Preparation [Active: Ursolic Acid] [135]
OAT3: (SLC22A8) [Brain-BBB; CSF-CRP; Neuron; Astrocyte]
ST: Homovanillic Acid; Dopamine; Noradrenaline; Adrenaline; Serotonin; Histamine;
Tryamine; Prostaglandin E2; Prostaglandin F2Alpha; cAMP; Cortisol; DHEAS; Epicatechin
3-O-(3-O-Methylgallate); Epicatechin; Epicatechin Gallate; Licuroside; Pongamoside;
Sulfaquinoxaline; Genistein-7-O-Glucuronide; Quercetin-3′-O-Glucuronide;
Glycitein-7-O-Glucuronide; Urate; Taurine; Creatinine; Estrone-3-Sulfate; Thymidine;
Intracellular Dicarboxylic Acid [e.g., AKG; Exchanged for Extracellular Organic Anions];
Rx{Famotidine, Cimetidine, Ranitidine} [33, 45, 46, 49, 50, 65, 109]
ITS: Caprylic Acid; AKG; Corticosterone; Camellia sinensis Preparation [Active: Epicatechin,
Epigallocatechin, Epichatechin-3-Gallate, Epigallocatechin-3-Gallate]; Rheum genus
Preparation [Active: Rhein, Chrysophanol, Physcion]; Punica granatum Preparation [Active:
Ursolic Acid, Gallic Acid]; Terminalia bellirica Preparation [Active: Gallic Acid]; Salvia
miltiorrhiza Preparation [Active: Lithospermic Acid, Rosmarinic Acid, Salvionolic Acid A,
Salvionolic Acid B, Tanshinol]; Ginkgo biloba Preparation [Active: 15:1 Ginkgolic Acid, 17:1
Ginkgolic Acid]; Glycyrrhizae genus Preparation [Active: 18Beta-Glycyrrhetinic Acid;
Glycyrrhizin (18Beta-Glycyrrhetinic Acid Precursor)]; Rx{Probenecid, Ranitidine} [33, 45, 46,
49, 50, 52, 63, 135, 242]
P-GP: (ABCB1) [Blood/Brain-BBB; CSF-CRP; Astrocyte; Microglia; Pericyte; Neuron]
ST: Quercetin; Kaempferol; Isorhamnetin; Pinocembrin; Bee Propolis Preparation [Active:
Pinocembrin]; Rx{Morphine, Verapamil, Dexamethasone, Loperamide}
ITS: Baicalin; Scutellaria genus Preparation [Active: Baicalin]; Berberine; Quercetin [1 mg/Kg
dose]; Silymarin; Silybum marianum Preparation [Active: Silymarin, Quercetin]; Procyanidine;
Curcumin; Borneol; Blumea balsamifera Preparation/Kaempferia galanga Preparation [Active:
Borneol]; Biochanin A; Trifolium pratense Preparation [Active: Biochanin A, Formononetin];
Chrysin; Passiflora genus Preparation [Active: Chrysin]; Flavone; Genistein; Glycine max
Preparation [Active: Genistein]; Pueraria mirifica Preparation [Active: Genistein]; Camellia
sinensis Preparation [Active: Epicatechin Gallate, Catechin Gallate, Epigallocatechin,
Epigallocatechin Gallate]; Hesperetin; Morin; Citrus junos Preparation/Citrus paradisi/sinensis
Preparation [Active: Naringenin]; Nobiletin; Isoquercitrin; Sophora japonica Preparation
[Active: Alpha-Glycosyl Isoquercitrin; Possible Inhibitor as Chemically Similar to
Isoquercitrin]; Kaempferol; Phloretin; Malus sylvestris/domestica Preparation [Active:
Phloretin]; Tangeretin; Bee Propolis Preparation [Active: Pinocembrin]; Ginkgo biloba
Preparation [Active: Ginkgolide B]; Pyrazine; Scillarenin; Betulinic Acid [Reduces P-GP
Amount]; Loperamide; Cinchona officinalis Preparation [Active: Quinine, Quinidine];
Rx{Quinidine} [52, 62, 105, 136, 137, 138]
ATS: Quercetin [0.1 mg/Kg dose]; Silybum marianum Preparation [Active: Quercetin];
Hypericum perforatum Preparation [Active: Hyperforin, Hypericin (Hypericin dose above 1
mg/human/day Increased P-GP Amount; Pregnane-X Receptor activator)]; Panax ginseng
preparation Preparation [150 mg/Kg]; Curcuma longa Preparation [Rhizome Extract]; Morinda
officinalis Preparation [Active: Bajijiasu (Increased P-GP amount)]; [NMDA-R
Activator/EP-1R Activator/Cyclooxygenase-2 Inhibitor/Increased Arachidonic Acid inside
BBB cell (Increases P-GP Amount)] [62, 123, 124, 134]
BCRP: (ABCG2) [Blood-BBB; CSF-CRP; Astrocyte; Pericyte]
ST: Xenobiotic [e.g., Various Drug(s)]; Folic Acid; Organic Anion
ITS: Chalcone; Biochanin A; Trifolium pratense Preparation [Active: Biochanin A,
Formononetin]; Genistein; Daidzein; Glycine max Preparation [Active: Genistein]; Pueraria
mirifica Preparation [Active: Daidzein, Genistein]; Chrysin; Passiflora genus Preparation
[Active: Chrysin]; Kaempferol; Hesperetin; Naringenin; Citrus junos Preparation/Citrus
paradisi/sinensis Preparation [Active: Naringenin]; Apigenin; Matricaria chamomilla
Preparation [Active: Apigenin]; Silibin; Quercetin; Silymarin; Silybum marianum Preparation
[Active: Silymarin, Quercetin]; Fisetin; Rhus succedanea Preparation [Active: Fisetin];
Phloretin; Malus sylvestris/domestica Preparation [Active: Phloretin]; Acacetin; Diosmetin;
Favone; Galangin; Kaempferide; Luteolin; Arachis hypogaea Preparation [Active: Luteolin];
Luteolin-4′-O-Glucoside; 7-Methoxyflavanone; Naringenin-7-Glucoside;
Theaflavin-3-O-Gallate; Theaflavin [137, 138]
ATS: Hypericum perforatum Preparation [Active: Hyperforin, Hypericin (Hypericin dose
above 1 mg/human/day Increased BCRP Amount)] [62]
MRP1: (ABCC1) [Blood-BBB/Brain-BBB; Blood-CRP; Astrocyte; Microglia; Pericyte]
ST: Leukotreine C4; Prostaglandin E2; GSH; GSSG; GSH Organic Anion Conjugates
ITS: Chrysoeriol; Diosmetin; 5,7,3′,4′-Tetramethoxyflavone; Robinetin; Kaempferol;
Tamarixetin; Isorhamnetin; 3′,4′-Dihydroxyflavone; Luteolin; Arachis hypogaea Preparation
[Active: Luteolin]; Baicalein; Scutellaria genus Preparation [Active: Baicalin, Baicalein];
Biochanin A; Trifolium pratense Preparation [Active: Biochanin A, Formononetin]; Chalcone;
Galangin; Kaempferide; Genistein; Glycine max Preparation [Active: Genistein]; Pueraria
mirifica Preparation [Active: Genistein]; Hesperetin; Morin; Phloretin; Malus
sylvestris/domestica Preparation [Active: Phloretin]; Quercetin; Silybum marianum Preparation
[Active: Quercetin]; Myricetin; Apigenin; Matricaria chamomilla Preparation [Active:
Apigenin]; Naringenin [Promotes GSH movement from inside to outside the cell; a relatively
low amount of GSH inside the cell reduces MRP1 movement of other substance (e.g.,
xenobiotic)]; Citrus junos Preparation/Citrus paradisi/sinensis Preparation [Active:
Naringenin]; Polmoric Acid [decreased MRP1 amount]; Curcuma longa Preparation [Extract
decreased MRP1 amount] [62, 137, 138]
MRP2: (ABCC2) [Blood-BBB; CRP]
ST: Xenobiotic; GSH; GSSG; Leukotriene C4; Organic Anion; Hydrophobic Substance;
Genistein-7-Glucoside; Quercetin; Silybum marianum Preparation [Active: Quercetin];
Quercetin-Glucuronide; Epicatechin
ITS: Myricetin; Quercetin-4′-Glucoside; Robinetin; Curcuma longa Preparation/Ginkgo biloba
Preparation [Decreased MRP2 amount] [62, 137, 138]
ATS: Hypericum perforatum Preparation [Active: Hyperforin, Hypericin (Hypericin dose
above 1 mg/human/day Increased MRP2 Amount)] [62]
MRP4: (ABCC4) [Blood-BBB/Brain-BBB; Blood-CRP/CSF-CRP]
ST: cAMP; cGMP; Prostaglandin E1; Prostaglandin E2; GSH; Folic Acid; Folinic Acid
ITS: Daidzin; Pueraria mirifica Preparation [Active: Daidzin]; Quercetin; Silybum marianum
Preparation [Active: Quercetin]; Hesperetin; Resveratrol; Polygonum cuspidatum Preparation
[Active: Trans-Resveratrol]; Naringenin; Citrus junos Preparation/Citrus paradisi/sinensis
Preparation [Active: Naringenin] [138]
MRP5: (ABCC5) [Blood-BBB; Brain CRP]
ST: cAMP; cGMP; GSH; Folic Acid; Organic Anion
ITS: Daidzin; Pueraria mirifica Preparation [Active: Daidzin]; Quercetin; Silybum marianum
Preparation [Active: Quercetin]; Naringenin; Citrus junos Preparation/Citrus paradisi/sinensis
Preparation [Active: Naringenin]; Hesperetin [138]
OATP1A2: (SLCO1A2) [Blood-BBB/Brain-BBB; Blood/Brain CRP]
ST: DHEAS; Estrone-3-Sulfate; Mesylate; Prostaglandin E2; Thyroid Hormone [e.g.,
Triiodothyronine, Thyroxine]; Rx{Acebutolol, Atenolol, Ciprofloxacin}
OATP2B1: (SLCO2B1) [Blood-BBB]
ST: DHEAS; Estrone-3-Sulfate; Pregnenolone Sulfate; Pivoxil; Hyperforin
OATP3A1V1: (SLCO3A1V1) [Blood-BBB/Brain-BBB; Blood/Brain CRP]
ST: Estrone-3-Sulfate; Prostaglandin E1; Prostaglandin E2; Vasopressin; Taurocholate;
Digoxin; DHEAS; Thyroid Hormone [e.g., Triiodothyronine, Thyroxine]; Arachidonic Acid
OATP3A1V2: (SLCO3A1V2) [Blood-CRP]
ST: Arachidonic Acid; Prostaglandin E1; Prostaglandin E2; Vasopressin; Taurocholate;
Digoxin; DHEAS; Thyroid Hormone [e.g., Triiodothyronine, Thyroxine]
HUMAN OATP1C1: (SLCO1C1) [BBB; Blood-CRP/CSF-CRP; Glial Cell]
ST: Estrone-3-Sulfate; Thyroid Hormone [e.g., Triiodothyronine, Thyroxine]
OCT1: (SLC22A1) [Blood-BBB/Brain-BBB; Brain CRP]
ST: Prostaglandin F2Alpha; Prostaglandin E2; Acetylcholine; Guanidine; Putrescine;
Agmatine; Spermine; Spermidine; Oryza sativa Preparation [Active: Spermidine]; Thiamine;
Carnitine; Cinchona officinalis Preparation [Active: Quinidine]; Rx{Lamotrigine, Cimetidine,
Quinidine}
ITS: Progesterone; Testosterone; Corticosterone; Acetylcholine; Guanidine; Agmatine;
Camellia sinensis Preparation [Active: Epigallocatechin Gallate]; Cinchona officinalis
Preparation [Active: Quinidine]; Rx{Cimetidine, Verapamil, Quinidine, Desipramine} [52, 63]
OCT2: (SLC22A2) [Blood-BBB/Brain-BBB; CSF-CRP; Neuron; Astrocyte]
ST: Prostaglandin F2Alpha; Prostaglandin E2; Serotonin; Acetylcholine; Agmatine; Spermine;
Spermidine; Oryza sativa Preparation [Active: Spermidine]; Choline; Adrenaline; Dopamine;
Creatinine; Histamine; Noradrenaline; Rx{Cimetidine, Famotidine}
ITS: Testosterone; Progesterone; Corticosterone; Agmatine; Creatinine; Guanidine;
Epigallocatechin Gallate; Rx{Cimetidine, Famotidine, Verapamil, Desipramine} [52, 63]
OCT3: (SLC22A3) [Brain-BBB; CSF-CRP; Neuron; Glial Cell; Astrocyte]
ST: Histamine; Adrenaline; Serotonin; Dopamine; Noradrenaline; Agmatine; Spermine;
Spermidine; Oryza sativa Preparation [Active: Spermidine]; Creatine; Homovanillic Acid
ITS: Corticosterone; Progesterone; Testosterone; Serotonin; Guanidine; Carnitine;
Rx{Cimetidine, Verapamil, Desipramine, Imipramine} [52]
VAChT: (SLC18A3) [Cholinergic Interneuron; Vesicle]
ST: Acetylcholine; Serotonin
ITS: Rx{Vesamicol}
NKCC1: (SLC12A2) [Neuron; Glial Cell]
ST: 2x Cl-, Na + , K + Transported Together
ITS: Rx{Bumetanide (Antagonist at a higher dose and Inhibits KCC2)} [149, 152]
KCC2: (SLC12A5) [GABAergic Interneuron; Glutamatergic Pyramidal Neuron]
ST: Cl-, K + Transported Together
ITS: NMDA-R Activation [Decreases KCC2 Phosphorylation that Decreases KCC2 Activity
and Cell Surface Amount and Increases KCC2 Protein Degradation]; BDNF [Decreases KCC2
Phosphorylation that Decreases KCC2 Activity and Cell Surface Amount]; Muscarinic
Acetylcholine Receptor Activation [Increases KCC2 Degradation]; Genistein [Decreases KCC2
Phosphorylation that Decreases KCC2 Activity]; Glycine max Preparation [Active: Genistein];
Pueraria mirifica Preparation [Active: Genistein]; Rx{Bumetanide (Antagonist)} [149, 150,
152, 153]
ATS: Piperine [TRPV1-R Agonist; and TRPV1-R activation Promotes KCC2 Protein
Expression]; Piper longum Preparation/Piper nigrum Preparation [Active: Piperine];
Resveratrol [Promotes KCC2 gene expression]; Polygonum cuspidatum Preparation [Active:
Trans-Resveratrol]; mGlu1-Receptor Activation [Increases KCC2 Phosphorylation by Ca2 +
Dependent PKC that Increases KCC2 Activity and Cell Surface Amount]; 5HT2A-R Activation
[Increases KCC2 Phosphorylation by Ca2 + Independent PKC that Increases KCC2 Cell
Surface Amount]; NMDA-R Inhibitor; Calpain [Protease that Degrades KCC2 Inhibitors] [149,
150, 153]
KCC3: (SLC12A6) [Glutamatergic Pyramidal/GABAergic Purkinje Neuron that has KCC2]
ST: C1-, K + Transported Together
ITS: Rx{Furosemide, Bumetanide} [152]
Na+—K+ ATPase:
ST: Na+, K+ Transported Together
ATS: Zanthoxylum Clava-herculis Preparation [Active: Chelerythrine (Inhibits PKC to
Promote Na+—K+ ATPase Activity)]
Large-Conductance Ca2+-Activated K+ Channel (“BK Channel”):
ST: K+
ATS: Ethanol [PAM] [146]
G-Protein-Coupled Inwardly Rectifying K+ Channel (“GIRK”):
ST: K+
ATS: Ethanol [PAM] [146, 147]
GLUT1: (SLC2A1) [Blood-BBB/Brain-BBB]
ST: Glucose; Dehydroascorbic Acid
CHT1: (SLC5A7)
ST: Choline
ATS: Coluracetam
VPAT: (SLC18B1) [Astrocyte; Neuron; Vesicle]
ST: Polyamine [e.g., Spermine, Spermidine]; Oryza sativa Preparation [Active: Spermidine];
Serotonin
ITS: Serotonin; Histamine [104]
ATS: Agmatine; Noradrenaline [104]
ENT1: (SLC29A1) [Brain-BBB; Blood-CRP]
ST: Pyrimidine Nucleoside [e.g., Thymidine, Cytidine, Uridine]; Purine Nucleoside [e.g.,
Adenosine, Guanosine, Inosine]
ITS: Uridine; Cytidine [132]
ATS: Gastrodia elata Preparation [Active: N6-(4-hydroxybenzyl)adenine Riboside)] [235]
ENT2: (SLC29A2) [Blood-BBB/Brain-BBB; Blood-CRP/CSF-CRP]
ST: Pyrimidine Nucleoside; Purine Nucleoside; Nucleobase [e.g., Adenine, Hypoxanthine,
Thymine, Uracil]
ITS: Uridine; Cytidine [132]
ENT3: (SLC29A3) [CRP]
ST: Pyrimidine Nucleoside; Purine Nucleoside; Nucleobase
CNT1: (SLC28A1)
ST: Pyrimidine Nucleoside [e.g., Uridine, Cytidine]
CNT2: (SLC28A2) [Blood-BBB; CSF-CRP]
ST: Purine Nucleosid [e.g., Adenosine, Inosine]; Uridine
CNT3: (SLC28A3) [Blood-CRP]
ST: Pyrimidine Nucleoside; Purine Nucleoside
VNUT: (SLC17A9) [Neuron; Glial Cell; Astrocyte; Bergmann Cell; Vesicle]
ST: ATP; ADP; AMP; GTP; UTP
ITS: Acetoacetate; Glyoxylate; Arachidonic Acid; Glycyrrhetinic Acid; Glycyrrhizae genus
Preparation [Active: 18Beta-Glycyrrhetinic Acid, Glycyrrhizin (18Beta-Glycyrrhetinic Acid
Precursor)]; GTP; UTP [82, 83, 88, 90, 92, 101]
PEPTI: (SLC15A1) [CRP]
ST: Dipeptide; Tripeptide
PEPT2: (SLC15A2) [CSF-CRP; Astrocyte]
ST: Dipeptide [e.g., Gly-Gln]; Tripeptide
PHT1: (SLC15A4)
ST: Histidine; Dipeptide; Tripeptide
MCT1: (SLC16A1) [Blood-BBB/Brain-BBB; Blood/CSF CLP; Glial Cell; Astrocyte;
Oligodendrocyte; Tanycyte]
ST: Ketone Body; Lactate; D-Lactate; Pyruvate; Dichloracetate; Gamma-Hydroxybutyrate;
Acetoacetate; Alpha-Ketobutyrate
ITS: Phloretin; Malus sylvestris/domestica Preparation [Active: Phloretin]; Quercetin; Silybum
marianum Preparation [Active: Quercetin]; Alpha-Ketoisocaproate; Alpha-Ketoisovalerate;
Apigenin; Matricaria chamomilla Preparation [Active: Apigenin]; Chrysin; Passiflora genus
Preparation [Active: Chrysin]; Biochanin A; Trifolium pratense Preparation [Active: Biochanin
A, Formononetin]; Fisetin; Rhus succedanea Preparation [Active: Fisetin]; Diosemin;
Hesperidin; Genicitin; Luteolin; Arachis hypogaea Preparation [Active: Luteolin]; Kaempferol;
Morin; Naringenin; Citrus junos Preparation/Citrus paradisi/sinensis Preparation [Active:
Naringenin] [54, 55, 56, 139]
ATS: Noradrenaline [Increases MCT Amount] [53]
MCT2: (SLC16A7) [Neuron; Glial Cell; Astrocyte; Tanycyte]
ST: Ketone Body; Lactate; Pyruvate; Acetoacetate
ITS: Phloretin; Malus sylvestris/domestica Preparation [Active: Phloretin]; Quercetin; Silybum
marianum Preparation [Active: Quercetin]; Alpha-Ketoisocaproate; Alpha-Ketoisovalerate [54,
55, 56]
ATS: Noradrenaline [Increases MCT Amount] [53]
MCT3: (SLC16A8) [Blood-CRP]
ST: Lactate
ATS: Noradrenaline; Glu with Gly; BDNF [Increases MCT Amount] [53, 54]
MCT4: (SLC16A3) [Glial Cell; Astrocyte; Tanycyte]
ST: Lactate; Ketone Body
ITS: Phloretin; Malus sylvestris/domestica Preparation [Active: Phloretin]; Quercetin; Silybum
marianum Preparation [Active: Quercetin] [54, 56]
ATS: Noradrenaline; BDNF/Nitric Oxide [Increases MCT Amount] [9, 53]
OGC: (SLC25A11) [Astrocyte; Mitochondria]
ST: Malate Exchanged for AKG
AGC1: (SLC25A12) [Neuron; Astrocyte; Mitochondria]
ST: Asp Exchanged for Glu
GC1: (SLC25A22) [Astrocyte; Mitochondria]
ST: Glu
MCT8: (SLC16A2) [BBB; CSF-CRP
ST: Thyroid Hormone [e.g., Triiodothyronine, Thyroxine]
OCTN1: (SLC22A4) [CRP]
ST: Ergothioneine; Stachydrine; Carnitine; Choline; Acetylcholine; Cinchona officinalis
Preparation [Active: Quinidine]; Rx{Cimetidine, Verapamil, Quinidine}
ITS: Carnitine; Choline; D-Carnitine; Cinchona officinalis Preparation [Active: Quinidine];
Rx{Cimetidine, Verapamil, Quinidine} [52]
OAT2: (SLC22A7) [CRP]
ST: DHEAS; Dopamine; Noradrenaline; Adrenaline; Serotonin; Prostaglandin E2;
Prostaglandin F2Alpha; Adenine; Adenosine; Cytidine; Guanidine; Guanosine; Inosine;
Thymine; Thymidine; cAMP; cGMP; Creatinine; Intracellular Glutamate Released/Exchanged
for Extracellular Organic Anion; Rx{Cimetidine, Ranitidine}
OAT4: (SLC22A11) [BBB]
ST: Prostaglandin E2; Estrone-3-Sulfate; Urate; Rx{Cimetidine}
ITS: Camellia sinensis Preparation [Active: Catechin] [135]
ATS: Glycyrrhizae genus Preparation [Active: 18Beta-Glycyrrhetinic Acid; Glycyrrhizin
(18Beta-Glycyrrhetinic Acid Precursor)] [135]
URAT1: (SLC22A12) [BBB; CRP]
ST: Urate; DHEAS; Lactate; Iodide; Bromide; Orotate; Nicotinic Acid; Intracellular Lactate
Exchanged for Extracellular Urate
MRP6: (ABCC6) [BBB; CRP]
ST: Peptide
CRT1: (SLC6A8) [Blood-BBB/Brain-BBB; Neuron]
ST: Creatine
ITS: Activator of PKC [Promotes CRT1 Moving from plasma membrane into the cell] [118]
TAUT: (SLC6A6) [Blood-BBB/Brain-BBB; Brain]
ST: Taurine; Beta-Ala
ITS: Beta-Ala; Ala; Pro; PKC Activator [Promotes TAUT Moving from plasma membrane into
the cell] [9, 48, 118]
BEST1: [Astrocyte]
ST: GABA; Glu
VGCC: [Astrocyte]
ST: Ca2+ [Generally Ca2 + Moved from Extracellular Space into Cell]
ATS: Activation of GABAA-R [166]
VRAC: [Astrocyte]
ST: GABA; Glu
1) Albrecht, J. and Zielinska, M. Neuropharmacology 2019 161: 107560; 2) Lewerenz, J. et al.
2013 18(5): 522-555; 3) Zaragoza, R. Front Physiol. 2020 11: 973; 4) del Amo, E. M. et al. Eur.
J. Pharm. Sci. 2008 35(3): 161-174; 5) Dolgodilina, E. et al. J. Cereb. Blood Flow Metab. 2016
36: 1929-1941; 6) Michalec, K. et al. Arch. Biochem. Biophys. 2014 554: 28-35; 7) Sun, P. et
al. Neurosci Lett. 2014 583: 32-36; 8) Nalecz, K. A. Front Cell Dev Biol. 2020 8: 594464; 9)
Broer, S. and Gether, U. Br J Pharmacol 2012 167(2): 256-278; 10) Mackenzie, B. and
Erickson, J. D. Pflugers Arch. 2004 447 (5): 784-795; 11) Fricke, M. N. et al. J Neurochem. 2007
102(6): 1895-1904; 12) Broer, A. and Brookes, N. J. Neurochem. 2001 77(3): 705-719; 13)
Zerangue, N. et al. J. Biol. Chem. 1995 270(12): 6433-6435; 14) Ryan, R. M. et al. Front Cell
Neurosci. 2021 15: 670346; 15) Li, Y. et al. Neuropharmacology. 2011 60(7-8): 1168-1175; 16)
Rowley, N. M. et al. Neurochemistry Int. 2012 61(4): 546-558; 17) Harrington, N. Journal of
Student Research 2012 1(1): 23-32; 18) Chen, T. et al. Glia. 2017 65(9): 1491-1150; 19) Yang,
Y. et al. J. Neurochem. 2004 88(6): 1361-1372; 20) Zielinska, M. et al. J. Neurochem. 2015
135(6): 1272-1281; 21) Scalise, M. et al. FEBS Lett. 2015 589: 3617-3623; 22)
Utsumoniya-Tate, N. et al. J. Biol. Chem. 1996 271: 14883-14890; 23) Hawkins, R. A. et al.
Am. J. Neurosci. Res. 2013 1: 1-25; 24) Schousboe, A. et al. Neurochem Res. 2014
39(10): 1980-1987; 25) Conti, F. et al. J. Comp Neurol. 1999 409(3): 482-494; 26) Conti, F. et
al. Brain Res Rev 2004 45: 196-212; 27) Schmitt, S. et al. ChemMedChem. 2015
10(9): 1498-1510; 28) Verrey, F. et al. Pflugers Arch 2004 447(5): 532-542; 29) Broer, S.
Pflugers Arch. 2014 466(1): 155-172; 30) Fotiadis, D. et al. Mol Aspects Med. 2013
34(2-3): 139-158; 31) Kanai, Y. et al. J Biol Chem 2000 275(27): 20787-20793; 32) Marc, D. T.
et al. Neurosci Biobehav Rev 2011 35(3): 635-644; 33) Ohtsuki, S. Biol Pharm Bull. 2004
27(10): 1489-1496; 34) Jones, R. S. and Morris, M. E. Clin Pharmacol Ther 2016
100(5): 454-463; 35) Guitart, K. et al. J. Neurochem. 2015 133(4): 558-571; 36) Chen, Y. and
Swanson, R. A. J. Neurochem. 2003 84(6): 1332-1339; 37) Kou, L. et al. Expert Opin Ther
Targets 2018 22(8): 715-726; 38) Lee, N.-Y. et al. Neurochem Res 2012 37(7): 1499-1507; 39)
Miecz, D. et al. J. Neurochem. 2008 104(1): 113-123; 40) Nakanishi, T. et al. J. Physiol. 2001
532(Pt 2): 297-304; 41) Hawkins, R. A. et al. FEBS Lett. 2006 580(18): 4382-4386; 42) Lee,
W. J. et al. J Biol Chem. 1996 271(32): 19129-19133; 43) Oppedisano, F. et al. Nutrients 2020
12(5): 143; 44) Eraly, S. A. et al., J Biol Chem. 2006 284(8): 5072-5083; 45) Riedmaier, A. E. et
al. Pharmacol. Rev 2012 64(3): 421-449; 46) Tsuruya, Y. et al. Drug Metab Dispos 2016
44(12): 1925-1933; 47) Shin, J. W., et al. Neurochem. Res. 2009 34: 1758-1766; 48) Palacin, M.
et al. 1998 78: 969-1005; 49) Grundemann, D. et al. J. Biol. Chem. 1998 273(47): 30915-30920;
50) Gasser, P. J. and Lowry, C. A. Horm Behav. 2018 104: 173-182; 51) Sanchez-Covarrubias,
L. et al. Curr Pharm Des. 2014 20(10): 1422-1449; 52) Koepsell, H. et al. Pharm Res. 2007
24(7): 1227-1251; 53) Bergersen, L. H. J Cereb Blood Flow Metab 2015 35(2): 176-185; 54)
Perez-Escuredo, J. et al. Biochem Biophys Acta 2016 1863(10): 2481-2497; 55) Halestrap, A. P.
Mol Aspects Med. 2013 34(2-3): 337-349; 56) Manning, J. E. et al J. Physiol. 2000 529(Pt
2): 285-293; 57) Broer, S. et al. Biochem. J. 1999 341(Pt 3): 529-535; 58) Samodelov, S. L. et
al. Int J Mol Sci. 2020 21(21): 7890; 59) Nigam, S. K. Annu Rev Pharmacol Toxicol 2018
58: 663-687; 60) Hong, S. S. et al. Pharmacol Res. 2007 56(6): 468-473; 61) Hosoya, K.-I. and
Tachikawa, M. Clin Exp Nephrol 2011 15(4): 478-485; 62) Khadka, B. et al. Int J Mol Sci 2021
22(4): 1809; 63) Lai, R. E. et al. J Food Drug Anal. 2018 26(2S): S45-S60; 64) Nakamura, Y. et
al. Biol Pharm Bull 2018 41(9): 1337-1347; 65) Shen, H. et al. Drug Metab Dispos 2017
45(2): 228-236; 66) Morris, M. E. et al. AAPS J. 2017 19(5): 1317-1331; 67) Hagglund, M. G. A.
et al. PLoS One 2013 8(3): e58651; 68) Hagglund, M. G. J. Biol. Chem. 2011 286(23): 20500-
20511; 69) Heja, L. et al. Neuropharmacology 2019 161: 107629; 70) Fattorini, G. et al. 2020
Front Cell Neurosci. 14: 9; 71) Melone, M. et al. Front. Neuroanat. 2014 8: 72; 72) Christiansen,
B. et al. J. Biol. Chem. 2007 282: 19331-19341; 73) Liu, Q. R. et al. J Biol Chem. 1993
268(3): 2106-2112; 74) Awad, J. A. et al. J. Biol. Chem. 1983 258(5): 2960-2965; 75)
Cristovao-Ferreira, S. et al. J. Neurochem. 2009 109(2): 336-347; 76) Borden, L. A. Neurochem
Int. 1996 29(4): 335-356; 77) Mederos, S. et al. Glia. 2019 67(10): 1842-1851; 78) Law, R. M. et
al. J. Biol. Chem. 2000 275(31): 23986-23991; 79) Yahyaoui, R. and Perez-Frias, J. Int J Mol
Sci 2019 21(1): 119; 80) Parra, L. A. et al. Mol Pharmacol 2008 74(6): 1521-1532; 81) Zaia,
K. A. and Reimer, R. J. J Biol Chem 2009 284(13): 8439-8448; 82) Juge, N. et al. Neuron 2010
68(1): 99-112; 83) Omote, H. et al. Ann Rev Pharmacol Toxicol 2016 56: 385-402; 84) Miyaji,
T. et al. Proc. Natl. Acad. Sci. U.S.A. 2008 105(33): 11720-11724; 85) Waldvogel, H. J. et al. J
Chem Neuroanat. 2019 97: 99-111; 86) Aubrey, K. R. Neurochem Int. 2016 98: 94-102; 87)
Juge, N. et al. J. Neurochem. 2013 127(4): 482-486; 88) Larsson, M. et al. Cereb. Cortex 2012
22(5): 1203-1214; 89) Sawada, K. et al. Proc Natl Acad Sci U.S.A. 2008 105(15): 5683-5686;
90) Kato, Y. et al. Biol Pharm Bull. 2013 36(11): 1688-1691; 91) Hasuzawa, N. et al. Biochim
Biophys Acta Biomembr. 2020 1862(12): 183408; 92) Miras-Portugal, M. et al. Front Cell
Neurosci. 2019 13: 224; 93) Takamori, S. et al. EMBO Res. 2002 3(8): 798-803; 94) Bai, L. et
al. J Biol Chem 2001 276(39): 36764-36769; 95) Takamori, S. et al. J. Neurosci 2001
21(22): RC182; 96) Erickson, J. D. et al. Proc. Natl. Acad. Sci. U.S.A. 1996 93(10): 5166-5171;
97) Lohoff, F. W. et al. Neuropsychopharmacology. 2006 31(12): 2739-2747; 98) Lohoff, F. W.
et al. Mol Psychiatry 2014 19(1): 129-139; 99) Eiden, L. E. FASEB J. 2000 14(15): 2396-2400;
100) Van Liefferinge, J. et al. Front Cell Neurosci 2013 7: 139; 101) Moriyama, Y. et al.
Purinergic Signal. 2017 13(3): 387-404; 102) Haas, H. L. et al. Physiol Rev. 2008
88(3): 1183-1241; 103) Eiden, L. E. et al. Pflugers Arch. 2004 447(5): 636-640; 104) Hiasa, M.
et al. Sci. Rep. 2014 4: 6836; 105) Eyal, S. et al. Phar Ther 2009 80-104; 106) Bodner, O. et al.
J Neurosci 2020 40(34): 6489-6502; 107) Neame, S. et al. Proc Natl Acad Sci USA 2019
116(41): 20736-20742; 108) Kristensen, A. S. et al. Pharmacol Rev 2011 63: 585-640; 109)
Yoshikawa, T. et al. Int J Mol Sci 2019 20(3): 737; 110) Pharmacology of Neurotransmitter
Release, “Pharmacology of Neurotransmitter Transport into Secretory Vesicles,” Sudhof, T. C.
and Starke, K. (Eds) 2008, Vol 184. Springer, Berline, Heidelberg; 111) Pizzagalli, M. D. et al.
FEBS J 2021 288(9): 2784-2834; 112) Amer, A. et al. Pharm Biochem Behav 2004 77: 137-143;
113) Hertz, L. and Rothman, D. L. Biology (Basel). 2017 6(1): 17; 114) Robinson, M. B. et al.
Neurochem Res. 2020 45(3): 526-540; 115) Engel, K. et al. J. Biol. Chem. 2004
279(48): 50042-50049; 116) Dahlin, A. et al. Neuroscience 2007 146(3): 1193-1211; 117)
Daws, L. C. et al. ACS Chem. Neurosci. 2013 4(1): 16-21; 118) Chen, N. H. et al. Pfueger Arch
Eur J Physiol 2004 447(5): 519-531; 119) Rosenberg, D. et al. J Neurosci 2013 33(8): 3533-
3544; 120) Maiya, R. et al. J Biol Chem 2002 277: 30724-30729; 121) Kaplan, E. et al. Proc
Natl Acad Sci USA 2018 115: 9628-9633; 122) Sherwood, M. W. et al. Int J Mol Sci 2021
22(14): 7258; 123) Qosa, H. et al. Brain Res. 2015 1628: 298-316; 124) Gil-Martins, E. et al.
Pharmacol Ther 2020 213: 107554; 125) Nicolussi, S. et al. Br. J. Pharmacol. 2020 177: 1212-
1226; 126) Stieger, B. and Gao, B. Clin Pharmacokinet. 2015 54(3): 225-242; 127) Virgintino,
D. et al. J Histochem Cytochem. 2002 50(12): 1671-1676; 128) Warren, M. S. et al.
Pharmacological Research. 2009 59(6): 404-413; 129) Daood, M. et al. Neuropediatrics. 2008
39(4): 211-218; 130) Huber, R. D. et al. Am J Physiol Cell Physiol. 2007 292(2): C795-C806;
131) Wu, W. et al. Drug Metab Dispos 2013 41: 1825-1834; 132) Kong, W. et al. Curr Drug
Metab. 2004 5(1): 63-84; 133) Redzic, Z. B. et al. et al. Cerebrospinal Fluid Research. 2010 7: 2;
134) Zibell, G. et al. Neuropharmacology. 2009 56: 849-855; 135) Wang, L. and Sweet, D. H.
Evid Based Complement Alternat Med 2013 2013: 612527; 136) Wong, C. C. et al. Biochem
Pharmacol 2011 81: 942-949; 137) Morris, M. E. and Zhang, S. Life Sci 2006 78: 2116-2130;
138) Brand, W. et al. Biomed Pharmacother 2006 60: 508-519; 139) Wang, Q. and Morris,
M.E. Drug Metab Dispos 2007 35: 201-208; 140) Ho, H. T. et al. J. Pharmacol. Exp. Ther. 2011
339: 376-385; 141) Ohtsuki, S. and Terasaki, T. Pharm Res. 2007 24: 1745-1758; 142) Cichon,
N. et al. Int J Mol Sci 2021 22(19): 10707; 143) Magi, S. Int J Mol Sci 2019 20(22): 5674; 144)
Niello, M. et al. Trends Pharmacol Sci 2020 41(7): 446-463; 145) Mori, M. et al. J. Physiol.
2002 539: 191-200; 146) Abrahao, K. P. et al. Neuron 2017 96(6): 1223-1238; 147) Lovinger,
D. M. and Roberto, M. Curr. Top. Behav. Neurosci 2013 13: 31-86; 148) Prado, V. F. et al.
Biochem. J. 2013 450(2): 265-274; 149) Hamidi, S. et al. Pflugers Arch 2015
467(11): 2325-2335; 150) Tang, B. L. Cells 2020 9(1): 240; 151) Malik, A. R. and Willnow, T. E.
Int J Mol Sci. 2019 20(22): 5671; 152) Josiah, S. S. et al. Int J Mol Sci. 2021 22(3): 1232; 153)
Kahle, K. T. et al. Trends Neurosci 2013 36(12): 726-737; 154) Blaesse, P. et al. 2009
61(2): 820-838; 155) Liu, R. et al. Front Neurol. 2020 10: 1407; 156) Woo, J. et al. Proc Natl
Acad Sci USA 2018 115(19): 5004-5009; 157) Yoon, B. E. et al. J Physiol 2014 592(22): 4951-
4968; 158) Sala-Rabanal, M. et al. Mol Pharm 2013 10(4): 1450-1458; 159) Skatchkov, S. N. et
al. Psychiatr Clin North Am. 2014 37(4): 653-678; 160) Dringen, R. et al. Neurochem Res. 2015
40(12): 2570-2582; 161) Baldo, B. A. et al. Br J Anaesth 2020 124(1): 44-62; 162) Sha, D. et al.
Neurochem Res 2004 29(1): 199-207; 163) Bridges, R. et al. Pharmacol Rev 2012
64(3): 780-802; 164) Kupchik, Y. M. et al. Biol Psychiatry 2012 71: 978-986; 165) Massie, A. et
al. J Neurochem. 2015 135(6): 1062-1079; 166) Caudal, L. C. et al. Front Cell Neurosci. 2020
14: 609947; 167) Solis, E. Neuroscience 2010 2: 82-89; 168) Jin, H. et al. Proc Natl Acad Sci
USA. 2003 100(7): 4293-4298; 169) Fei, Y. et al. Front Neurol. 2020 11: 592514; 170) Heja, L.
et al Dobolyi BMC Biol. 2012 10: 26; 171) Koyama, Y, et al. J Neurosci Res 2000 59: 685-691;
172) Doyon, N. et al. Neuron 2016 89(6): 1157-1172; 173) Cichon, N. et al. Int J Mol Sci 2021
22(19): 10707; 174) Lin, L. et al. Nat Rev Drug Discov 2015 14(8): 543-560; 175) Campos, F. et
al. Int J Biochem Cell Biol 2012 44(2): 262-265; 176) Miller, D. K. et al. J Pharmacol Exp Ther
2004 310(3): 1035-1045; 177) Crooks, P. A. et al. Curr Top Med Chem 2011 11(9): 1103-1127;
178) Wilhelm, C. J. et al. Biochem Pharmacol. 2008 75(6): 1411-1215; 179) Conti, F., et al. J
Comp Neurol. 1998 396(1): 51-63; 180) Schousboe, A. Neurochem Res 2000 25, 1241-1244;
181) Melone, M. et al. J. Chem. Neuroanat. 2005 30(1): 45-54; 182) DeFelipe, J. and
Gonzalez-Albo, M. C. NeuroReport 1998 9(3)467-470; 183) Oenarto, J. et al. Arch Biochem
Biophys 2014 560: 59-72; 184) Fuhrer, T. E. et al. Neuroscience. 2017 351: 108-118; 185) Pow,
D. V. et al. Cell Tissue Res. 2005 320(3): 379-392; 186) Song, I. et al. Front Cell Neurosci 2013
7: 23; 187) Lia, A. et al. Neurosci Lett 2019 689: 14-20; 188) Olsen, R. W. Neuropharmacology
2018 136(Pt A): 10-22; 189) Zavala-Tecuapetla, C. et al. Int J Mol Sci 2020 21(22): 8573; 190)
Doyon, N. et al. PLOS Comput. 2011 Biol. 7(9): e1002149; 191) Lee, S. et al. Science 2010
330(6005): 790-796; 192) Yoon, B.-E. et al. Mol. Brain 2011 4: 42; 193) Lee, M. et al. Glia.
2011 59(11): 1600-1611; 194) Freitas, A. E. et al. Prog. Neuropsychopharmacol. Biol.
Psychiatry 2014 50: 143-150(B); 195) Jo, S. et al. Nat Med 2014 20: 886-896; 196) Bergin,
D. H. et al. Sci Rep 2019 9(1): 12669; 197) Freitas, A. E. et al. J. Psychiatr. Res. 2014 58: 137-
146(A); 198) Weng, X. et al. Acta Pharmacol. Sin. 2003 24: 746-750; 199) Olmos, G. et al. Br.
J. Pharmacol. 1999 127: 1317-1326; 200) Moosavi, F. et al. Drug Des Devel Ther 2015
10: 23-42; 201) Melendez, R. I. et al. J Pharmacol Exp Ther 2005 314: 139-147; 202) Sepkuty,
J. P. et al. J Neurosci. 2002 22(15): 6372-6379; 203) Storck, T. et al. Proc Natl Acad Sci USA
1992 889(22): 10955-10959; 204) Takahashi, K. et al. Cell Mol Life Sci. 2015
72(18): 3489-3506; 205) Daikhin, Y. and Yudkoff, M. J Nutr. 2000 130(4S
Suppl): 1026S-1031S; 206) Schousboe, A. and Divac, I. 1979 Brain Res. 177(2): 407-409; 207)
Rothstein, J. D. et al. Neuron 1996 16(3): 675-686; 208) Mahmoud, S. et al. Cells. 2019
8(2): 184; 209) Lehre, K. P. and Danbolt, N. C. J. Neurosci. 1998 18(21): 8751-8757; 210)
Mathews, G. C. and Diamond, J. S. J Neurosci. 2003 23(6): 2040-2048; 211) Bak, L. K. et al. J
Neurosci Res. 2012 90(9): 1768-1775; 212) Trudeau, L. E. and Mestikawy, S. E. Front Neural
Circuits. 2018 12: 113; 213) Munster-Wandowski, A. et al. Front Synaptic Neurosci. 2016 8: 4;
214) Mederos, S. et al. Front Synaptic Neurosci. 2018 10: 45; 215) Bernstein, A. I. et al.
Neurochem Int. 2014 73: 89-97; 216) German, C. L. et al. Pharmacol Rev. 2015
67(4): 1005-1024; 217) Ahnert-Hilger, G. and Jahn, R. Nat Neurosci. 2011 14(4): 405-407; 218)
Jahn, R. and Fasshauer, D. Nature. 2012 490(7419): 201-207; 219) Brose, N. Traffic 2008
9(9): 1403-1413; 220) Weinert, S. et al. EMBO J. 2020 39(9): e103358; 221) Riazanski, V. et al.
Nat Neurosci. 2011 14(4): 487-494; 222) Angulo, M. C. et al. J. Neurosci. 2004 24(31): 6920-
6927; 223) Harada, K. et al. Front. Neurosci. 2016 12(9): 499; 224) Li, Y. et al. Mol Med Rep
2014 9(1): 305-310; 225) Teichberg, V. I. et al. Neuroscience 2009 158: 301-308; 226) Boyko,
M. et al. J. Neural Transm (Vienna) 2014 121(8): 971-979; 227) Tsai, P. J. and Huang, P. C. J.
Nutr. 2000 130(4S Suppl): 1002S-1004S; 228) Smith, Q. R. et al. J. Neurochem. 1987
49: 1651-1658; 229) Hertz, L. Neurochem Int. 2004 45 (2-3): 285-296; 230) Turecki, G.
Molecular Psychiatry 2013 18(12): 1242-1243; 231) Smith, Q. R. J. Nutr. 2000 130(4S
Suppl): 1016S-1022S; 232) Yoshitake, T. et al. Br J Pharmacol. 2010 159(3): 659-668; 233)
Fehske, C. J. et al. Pharmacol Res. 2009 60(1): 68-73; 234) Nickell, J. R. et al. Adv Pharmacol.
2014 69: 71-106; 235) Huang, N.-K. et al. PLOS One 2011 6(6): e20934; 236) Nakatani, Y. et al.
Eur J Neurosci. 2008 27(9): 2466-2472; 237) Rahman, M. K. et al. Jpn J Pharmacol. 1982
32(5): 803-811; 238) Barnes, J. et al. Herbal Medicines, 3rd Edition Pharmaceutical Press,
London 2007; 239) Firstova, Y. Y. et al. Neurochemical J. 28(2): 130-141; 240) European Patent
Application no. EP2891491A1; 241) Sahoo, S. and Brijesh, S. Biomed Pharmacother 2019
109: 1148-1162; 242) Ref. 116, Table 4; 243) Xu, S. et al. Front Pharmacol 2020 11: 508

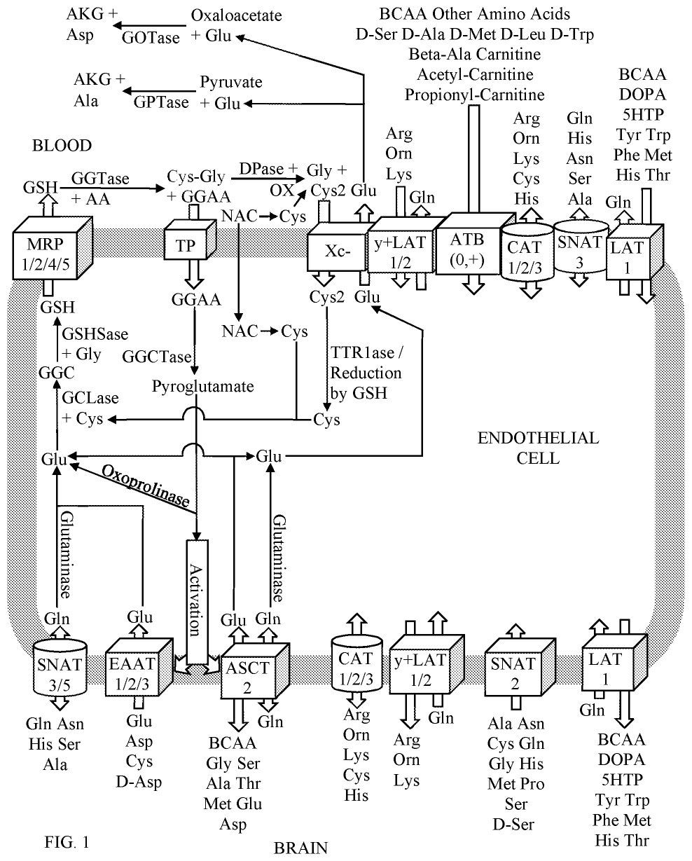

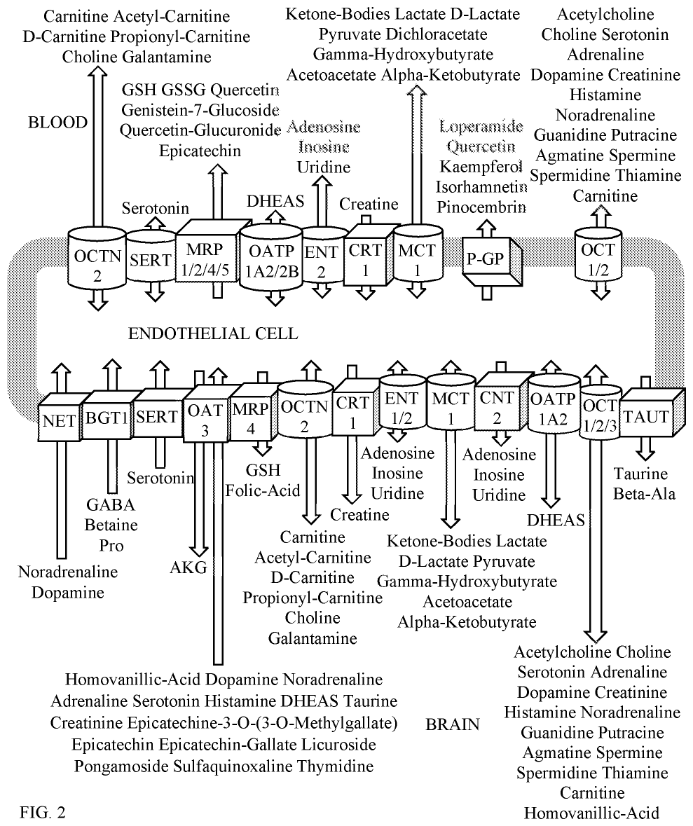

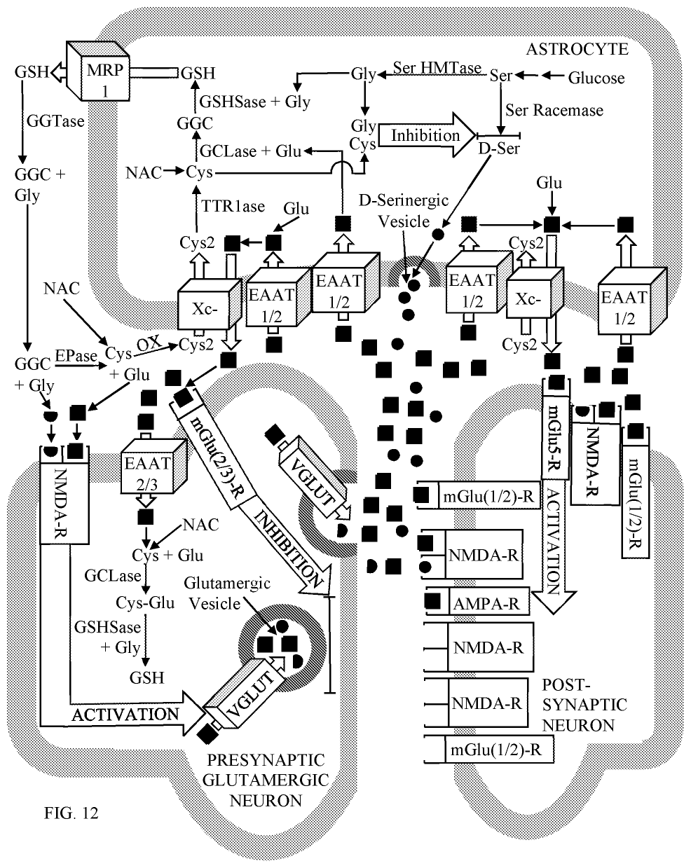

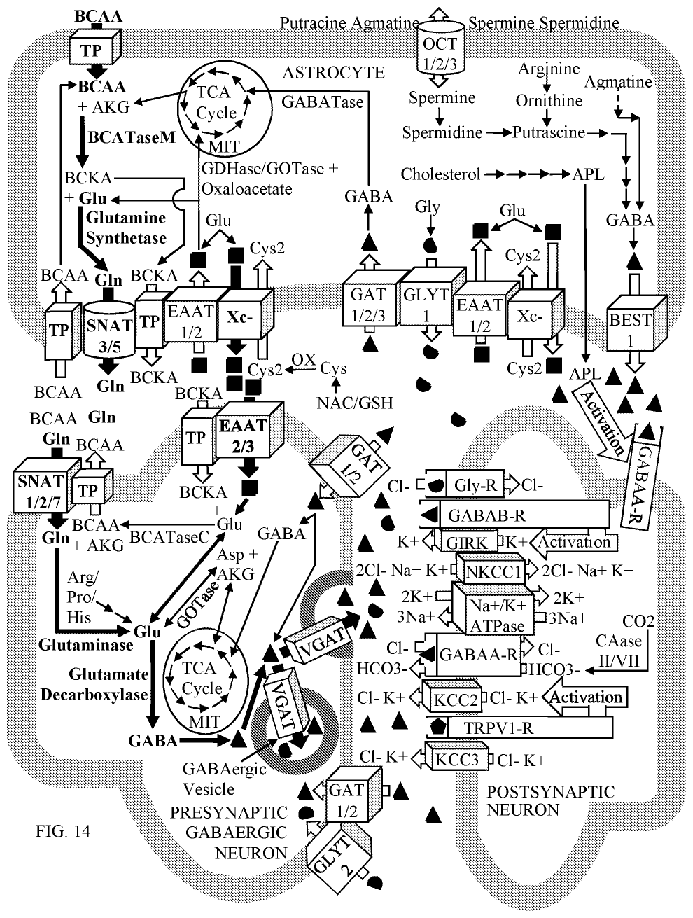

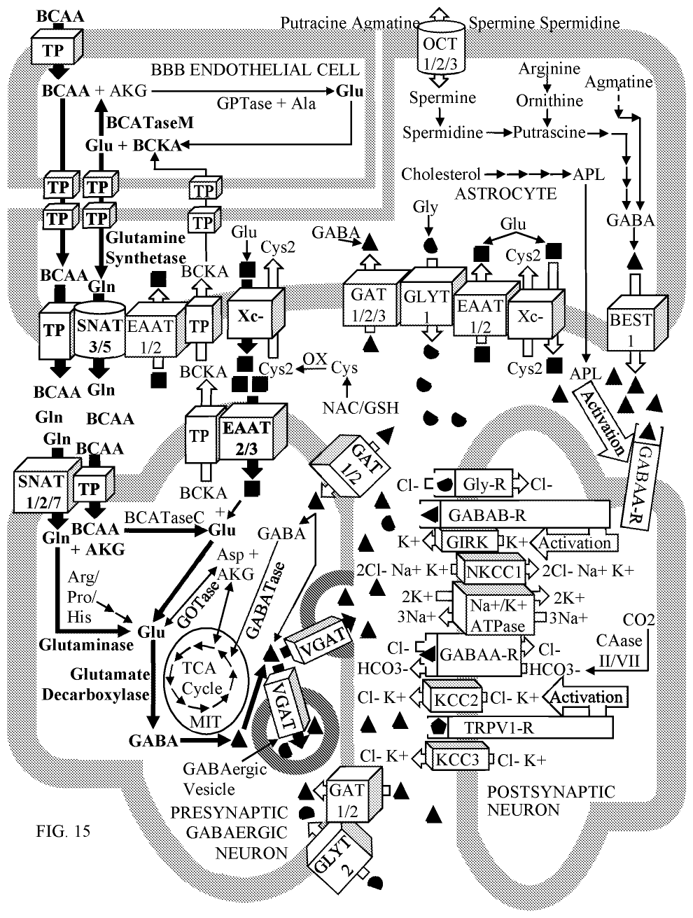

Tables 1, 2, 3 and 4 show the transporters, receptors, and enzymes involved in glutathione creation and/or degradation and related reactions, neurotransmitter signaling, and the various activator and inhibitor treatment substances for these proteins, with many of these proteins depicted in FIG. 1, FIG. 2, FIG. 12, FIG. 14, and FIG. 15. FIG. 1 and FIG. 2 depict transporters (“TP”) that mediated movement of substances (e.g., treatment substances) across the BBB and possible general direction of movement of the substances (e.g., into the brain, into the blood) shown by arrows, and Table 1 and Table 2 describe the transporters. As used herein, more than one subtype of transporter is depicted as the number/letter of the subtypes separated by the “and/or” symbol “/” under the type of transporter (e.g., FIG. 1, at the bottom left, the SNAT3 and SNAT5 transporters are depicted as “SNAT 3/5”). The main inhibitory neurotransmitter is gamma-aminobutyric acid (“GABA”) and main excitatory neurotransmitter is Glu (though the amount of GABA is about 20-fold less than Glu in the brain), and GABA and Glu (and many other neurotransmitters) are produced/degraded by enzymes from substances moved across the BBB and/or between brain neurons, astrocytes, and/or BBB endothelial cells [Ref 10, Table 6]. For example, a treatment substance may alter GABAergic and/or glutamatergic responses by altering (e.g., increasing, decreasing) extracellular GABA concentration available to bind a GABA receptor and/or extracellular Glu concentration available to bind a Glu receptor.

For mammals, as depicted in FIG. 1, Gln/Glu is moved from the brain into BBB endothelial cells by transporters such as ASCT2 and/or EAAT1/2/3; and endothelial cells glutaminase converts Gln into Glu that is enzymatically converted into GSH. GSH is moved by MPR1/2/4/5 into the blood, and GSH is enzymatically converted into cysteineylglycine (“Cys-Gly”) and gamma-glutamyl-amino acid (“GGAA”), and GGAA is moved by a transporter (“TP”) into the BBB endothelial cell and enzymatically converted into pyroglutamate. Pyroglutamate activates transporters (e.g., EAAT1/2/3, ASCT2) to promote movement of Glu into the BBB endothelial cell.

FIG. 1 depicts a model where the Cys prodrug NAC is hydrolyzed into Cys that may be converted in the blood by oxidation (“OX”) into Cys2. Cys2 is moved into the BBB endothelial cell by the Xc- transporter in exchange for moving Glu from the BBB endothelial to the blood, reducing the brain's Glu amount while providing Cys for GSH production. Pyruvate increase Glu conversion into AKG and alanine by blood glutamate-pyruvate transaminase (“GPTase”) to promote movement of Glu from the brain across the BBB into the blood [Ref. 225, Table 2]. For mammals, oxaloacetate injected into the blood will increase Glu conversion into AKG and Asp in the blood by glutamate oxaloacetate transaminase (“GOTase”) located in the blood; reducing the amount of Glu in the blood, and this mechanism promotes movement of Glu from the brain. Injection of oxaloacetate, pyruvate, and/or lipoamide, decreased the blood's and brain's Glu amount for about an hour in mammals [Ref. 113, 160, 175, and 224, Table 2; Ref 1, Table 3].

NAC readily enters the endothelial cell without the need for a transporter, and conversion into Cys promotes the production of GSH comprising Glu that is moved into the blood. The effect of an increased amount of Cys/Cys2 in a BBB endothelial cell/blood is the increased movement of Glu and Gln into the BBB endothelial cell and increased movement of Glu out of the BBB endothelial cell and into the blood. A positive cysteineic treatment substance (e.g., NAC) increases in Cys/Cys2 in the BBB endothelial cell/blood that may reduce the amount of Glu excitatory transmitter in the brain, and the positive cysteineic may be combined with another treatment substance (e.g., oxaloacetate, pyruvate) that promote the conversion of Glu to other chemicals in the blood to reduce the Glu amount in the brain.

TABLE 3
Enzymes Involved in Glutathione (“GSH”) Metabolism
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Thioredoxin Reductase 1: (EC 1.8.1.9; “TTR1ase”)

| | |
|---|---|
| S: Cys2 | P: Cys |

Glutamate Cysteine Ligase: (EC 6.3.2.2; “GCLase”) [Astrocyte; Neuron; BBB Endothelial
Cell]

| | |
|---|---|
| S: Cys + Glu | P: Gamma-Glutamylcysteine (“GGC”) |

ATS: Epigallocatechin Gallate/Alpha-Lipoic Acid [Increases mRNA Expression to Increase
Intercellular GSH] [8, 9]
Glutathione Synthase: (EC 6.3.2.3; “GSHSase”) [Astrocyte; Neuron; BBB Endothelial Cell]

| | |
|---|---|
| S: GGC + Gly | P: GSH |

Gamma-Glutamyltransferase: (EC 2.3.2.2; “Gamma-Glutamyl Transpeptidase,” “GGTase”)
[Astrocyte/Cell Plasma Membrane Facing the Extracellular Space]

| | |
|---|---|
| S: GSH + Amino Acid (“AA”) | P: Cysteineylglycine (“Cys-Gly”) + |
| | Gamma-Glutamyl-Amino Acid (“GGAA”) |
| S: GSH + Amino Acid (“AA”)/Water | P: Gamma-Glutamylcysteine + Gly |

Dipeptidase: (“DPase”; e.g., Ectopeptidase)

| | |
|---|---|
| S: Cys-Gly | P: Cys + Gly |

Ectopeptidase: (“EPase”) [Neuron′s Plasma Membrane Facing the Extracellular Space]

| | |
|---|---|
| S: Cys-Gly | P: Cys + Gly |
| S: Gamma-Glutamylcysteine | P: Glu + Cys |

Gamma-Glutamyl Cyclotransferase: (EC. 2.3.2.4; “GGCTase”)

| | |
|---|---|
| S: GGAA | P: Pyroglutamate + AA |

5-Oxoprolinase: (EC 3.5.2.9; “Pyroglutamase”)

| | |
|---|---|
| S: Pyroglutamate | P: Glu |

Alanine Aminotransferase: (EC 2.6.1.2; “GPTase,” “Amino Transaminase,”
“Glutamate-Pyruvate Transaminase”; Cofactor: Pyridoxal 5′ Phosphate) [Blood; Astrocyte;
Neuron, Where Reverse Reaction May Dominate in Neuron]

| | |
|---|---|
| S: Glu + Pyruvate | P: AKG + Ala |

ITS: Zanthoxylum Clava-herculis Preparation [Active: Chelerythrine]
Glutamate Oxaloacetate Transaminase: (EC 2.6.1.1; “Aspartate Transaminase,” “GOTase”;
Cofactor: Pyridoxal 5′ Phosphate) [Neuron; Astrocyte; Blood; Mitochondria]

| | |
|---|---|
| S: Glu + Oxaloacetate | P: AKG + Asp |

ITS: Zanthoxylum Clava-herculis Preparation [Active: Chelerythrine]
ATS: Oxaloacetate in Blood [Reduces Blood Glu Levels Allowing More Movement of Glu
Across the BBB from the Brain into the Blood]
Glutaminase: (EC 3.5.1.2) [Neuron, BBB Endothelial Cell]

| | |
|---|---|
| S: Gln | P: Glu |

ITS: Glu (Feedback Inhibitor)
ATS: Adenosine; Dinucleotide; Trinucleotide (e.g., ATP); Succinate; Citrate
Glutathione Peroxidase: (EC 1.11.1.9) and/or Chemical Reactions with Free Radicals

| | |
|---|---|
| S: GSH + GSH | P: GSSG |

Glutathione Reductase: (EC 1.8.1.7; Cofactor: NADPH)

| | |
|---|---|
| S: GSSG | P: GSH + GSH |

1) Cooper, A. J. L. and Jeitner, T. M. Biomolecules. 2016 6(2): 16; 2) Ref. 35, Table 2; 3) Ref. 23, Table 2; 4) Ref. 41, Table 2; 5) Hawkins, R. A. and Vina, J. R. Biology (Basel). 2016 5(4): 37; 6) Ref. 2, Table 2; 7) Ref. 160, Table 2; 8) Howes, M.-J. R. et al. Br. J. Pharmacol. 2020 177: 1294-1315; 9) Holmquist, L. et al. Pharmacol. Ther. 2007 113: 154-164; 10) Zhou, J. et al. Nat Rev Drug Discov 2005 4: 1015-1026; 11) Lavoie, S. et al. Neuropsychopharmacology 2008 33: 2187-2199; 12) Kau, K. S. et al. Neuroscience 2008 155: 530-537; 13) Moran, M. M. et al. J Neurosci 2005 25: 6389-6393; 14) Moussawi, K. et al. Nat Neurosci 2009 12: 182-189; 15) Williams, L. E. et al. J Neurosci. 2014 34: 16093-16102; 16) Warr, O. et al., J Physiol. 1999 Feb. 1; 514(Pt 3): 783-783; 17) Soria, F. N. et al J Clin Invest. 2014 124(8): 3645-3655; 18) Gallagher, M. Epilepsy Curr. 2020 20(1): 39-42; 19) Tardiolo, G. et al. Molecules 2018 23(12): 3305

Once a treatment substance enters the brain the treatment substance may interact with and/or become a component of human neurological/biological function to alter TSF and/or another sexual function (e.g., improve TSF, improve ease of ejaculation, etc.) as well as determine the interaction of component(s) in human neurological/biological function by the effect caused by the treatment substance. Examples of a component of human neurological/biological function include a neurotransmitter, a chemical involved in a neurotransmitter's synthesis and/or degradation, a transporter, an enzyme, and/or a metabolic process related to neurotransmission.

A neurotransmitter typically is a chemical typically inside a neurological cell (e.g., a neuron, a glial cell) wherein the chemical is released into the extracellular space where the chemical either is, or is converted into (e.g., enzymatically altered), an agonist for the main site of a neurotransmitter receptor. An example of a chemical enzymatically converted into a neurotransmitter after release adenosine, which is produced from the released neurotransmitter ATP, and both bind different neurotransmitter receptors. A junction between two or more neurons is referred to a “synapse.” A neurotransmitter often is released from one neuron (e.g., a presynaptic neuron) to diffuse across the synapse through a small space between two neurons referred to a “synaptic cleft” to contact a neurotransmitter receptor facing the synaptic cleft, where the neurotransmitter receptor is part of another cell (e.g., a postsynaptic neuron, a glial cell). As used herein “extrasynaptic” refers to any region of a cell (e.g., a neuron, an astrocyte) other than the synaptic cleft. Examples of neurotransmitters typically released by a neuron include Glu, D-Ser, Gly, GABA, serotonin, dopamine, adrenaline, noradrenaline, histamine, acetylcholine, anandamide, 2-arachidonoyl glycerol, ATP, ADP, adenosine, and nitric oxide. Neurons that release and synapses that use one or more neurotransmitter(s) are typically named for the neurotransmitter(s), such as a “GABAergic” neuron that releases GABA, a “Cholinergic” synapse that has acetylcholine as a prominent neurotransmitter, etc. For example, about 20% of brain neurons are GABAergic. Neurological cells that synthesize and/or release a specific neurotransmitter often preferentially transport into those neurological cells the neurotransmitter after release (“reuptake” of the neurotransmitter) and/or a chemical that is used to synthesize the specific neurotransmitter. For example, for rats' corpus striatum, cholinergic neurons are about 1% of brain cells but transport about 60% of extracellular choline into the cholinergic neurons to be metabolized into the neurotransmitter acetylcholine for release by the cholinergic neurons. Examples of a neurotransmitter (and a transporter that reuptakes the neurotransmitter) include: serotonin (SERT, PMAT, OCT3); dopamine (DAT); noradrenaline (NET); histamine (PMAT); Glu (EAAT1/2/3); Gly (GLYT2); D-Ser (SNAT1/2); polyamine (OCT1/2/3); GABA (GAT1); and adenosine (CNT/ENT). Examples of a neurotransmitter (and a transporter that reuptakes and/or releases the neurotransmitter) include: D-Ser (ASC1); GABA (GAT2/3); and Gly (GLYT1). Examples of a glial cell (e.g., astrocyte) released neurotransmitter (“gliotransmitter”) include GABA; Glu; D-Ser; Gly; a nucleotide such as ATP; an organic acid such as homocysteic acid, taurine, lactic acid; BDNF; and a peptide such as atrial natriuretic peptide. A neuron/glial cell can also release a neuropeptide that is a peptide larger than most neurotransmitters, but are referred to herein as a neurotransmitter. Zinc ions (“Zn2+”) are moved into and released from synaptic vesicles of GABAergic, glycinergic, and glutamatergic neurons, and the Zn2+ binds receptors to modify the activity of neurotransmitter receptors. Zn2+ may be considered herein a neurotransmitter but will typically be described as a PAM or NAM of a neurotransmitter receptor as appropriate.

A neurotransmitter receptor is the minimum number of protein(s) wherein the binding of a neurotransmitter to the neurotransmitter receptor causes a change in the cell (e.g., the opening and/or closing protein ion channels on the plasma membrane). For example, a GABAA-R has 5 protein subunits, typically 2 Alpha subunits (e.g., Alpha1 Subunit), 2 Beta subunits (e.g., Beta2 Subunit), and a Gamma subunit, though others have a Delta subunit rather than Gamma subunit. The main binding site for an agonist/antagonist is formed between an Alpha subunit and a Beta subunit. Examples of allosteric modulator sites for the GABAA-R include the benzodiazepine binding site, generally a PAM site, that is formed between an Alpha subunit and Gamma subunit; the ethanol binding site and the etomidate/propofol binding site, both generally anesthetic sites, that are between an Alpha subunit and Beta subunit; a barbiturate/propofol/picrotoxin binding site, generally a NAM site, may be between an Alpha subunit and Beta subunit and/or a Beta subunit and Gamma subunit; and neurosteroid sites, that are generally PAM sites, thought to be on a GABAA-R having Alpha/Beta/Gamma subunits. In another example, the GABAARho-R typically has 5 Rho subunits, and is considered herein as a subtype of a GABAA-R. In an additional example, the GABAB-R has 2 subunits of: a GABAB1 subunit (e.g, GBAAB1A or GABAB1B subunit) and a GABAB2 subunit [Ref. 33, Table 14; Ref. 146, 147, 149, 152, 172, 188, 189, and 190, Table 2; Ref. 125, Table 4]

A neurotransmitter receptor is typically located on the exterior plasma membrane of a cell, though some neurotransmitter receptors are located inside a cell. Examples of neurotransmitter receptors typically located on a cell's plasma membrane include an ionotropic (“ion channel-linked”) receptor; a metabotropic (“G protein-linked”) receptor; a kinase linked receptor and/or an enzyme-linked hormone receptor. For example, neuropeptides such as galanin (e.g., 30 amino acids), galanin-message associated peptide, alarin, and galanin-like peptide, are agonists for galanin receptors Gal-R1/Gal-R2/Gal-R3, and activation of the plasma membrane located metabotropic Gal-R2 receptor on noradrenergic/serotoninergic neurons promotes Ca2+ release from the intracellular endoplasmic reticulum to promote neurotransmitter release. Increased intracellular Ca2+ promotes neurotransmitter release in glial cells (e.g., astrocytes)/neurons by vesicle/non-vesicle mechanisms. Examples of neurotransmitter receptors located inside a cell include a cytoplasmic receptor and/or a nuclear receptor.

A neurotransmitter and a neurotransmitter receptor are typically classified as excitatory or inhibitory, though some may have both activities depending upon the location on a cell. Neurotransmitters that typically have excitatory activity include Glu; acetylcholine; a catecholamine such as dopamine, adrenaline, and noradrenaline; and/or a monoamine such as dopamine, serotonin, adrenaline, noradrenaline. The binding of an excitatory neurotransmitter to an excitatory neurotransmitter receptor promotes the cell's (e.g., a neuron's) internal plasma membrane surface to become more positively charged (“depolarized,” “excitatory potential”), typically by opening plasma membrane ion channels for positively charged ions [e.g., a potassium ion (“K+”), a calcium ion (“Ca2+”), a sodium ion (“Na+”), a magnesium ion (“Mg2+”)] to promote movement of the positively charged ions into the cell. Neurotransmitters that typically have inhibitory activity include GABA and Gly. The binding of an inhibitory neurotransmitter to an inhibitory neurotransmitter receptor promotes the cell's (e.g., a neuron's) internal plasma membrane surface to become more negatively charged (“hyperpolarized,” “inhibitory potential”), typically by opening plasma membrane ion channels for negatively charged ions [e.g., a chloride ion (“Cl−”)], to promote movement of the negatively charged ions into the cell. For example, activation of the Gal-R1/Gal-R3 receptor(s) on a neuron opens a K+ channel to release K+ to extracellular space to hyperpolarize the neuron.

Neurons and astrocytes can release more than one neurotransmitter at a time to produced mixed excitory/inhibitor signaling, and often neurotransmitter receptors located at extrasynaptic sites will have the opposite response to the neurotransmitter relative to neurotransmitter receptors located in the synaptic cleft to reduce synaptic signaling when an excessive amount of an extracellular neurotransmitter is present. For example, excess neurotransmitters diffusing from the synaptic cleft may: activate an extrasynaptic neurotransmitter receptor on a presynaptic neuron that reduces release of the neurotransmitter from the presynaptic neuron and/or may activate an extrasynaptic neurotransmitter receptor on a postsynaptic neuron that opens/closes ion channels to reduce the synaptic signaling of the neurotransmitter. In another example, excess neurotransmitters diffusing from the synaptic cleft may activate a neurotransmitter receptor on an astrocyte that promotes neurotransmitter released by the astrocyte in an extrasynaptic region of a presynaptic/postsynaptic neuron to decrease (or in some instances increase) synaptic neurotransmitter signaling [Ref 20, 23, 90, 91, 92, 93, 94, 95, and 96, Table 4; Ref 5, Table 5; Ref 77, Table 2]. The neurotransmitter receptors may also produce mixed signaling when interacting. For example, a heteromer is two or more different types of proteins having different functions (e.g., two or more different neurotransmitter receptors) physically contacting each other, often to promote allosteric interactions between the different proteins. An example of a heteromer is an adenosine A2A-Rs and dopamine D2-Rs and adenylyl cyclase heteromer. Binding of adenosine to the A2A-Rs allosterically inhibits the activity of the neurotransmitter dopamine binding the dopamine D2-Rs of the heteromer; and binding of dopamine to the dopamine D2-Rs allosterically inhibits the activity of adenosine binding the A2A-Rs that inhibits activation of the adenylyl cyclase of the heteromer [Ref 87, 88, and 89, Table 4; Ref 6, Table 14].

A treatment substance that activates a neurotransmitter synaptic signaling pathway may be referred to herein and claimed as an activating/positive treatment substance for that neurotransmitter signaling pathway (e.g., “activating cholinergic treatment substance,” “positive GABAergic treatment substance,” “positive glutamatergic”). A treatment substance activating a neurotransmitter signaling pathway may be an inhibitory treatment substance to a specific protein in that pathway, such as, for example, it is contemplated that an acetylcholinesterase inhibitory treatment substance reducing the degradation of acetylcholine by acetylcholinesterase to promote increased the acetylcholine amount in the extracellular synaptic space, and the acetylcholine promotes activation of synaptic acetylcholine receptors. A treatment substance activating a neurotransmitter signaling pathway may be an activating treatment substance to a specific protein in that pathway, such as, for example, a branched-chain amino acid being a positive GABAergic treatment substance, as it is contemplated that an ingested branched chain amino acid is metabolized by several enzymes into GABA that is released into the extracellular space, and the increased amount of GABA activate synaptic GABA receptors. A treatment substance inhibiting a neurotransmission signaling pathway may be re referred to herein and claimed as an inhibiting/negative treatment substance for that neurotransmitter signaling pathway. For example, it is contemplated that a treatment substance that increases extrasynaptic Glu, such as a positive cysteineic, may activate a mGlu2/mGlu2 extrasynaptic receptor on a presynaptic neuron to reduce the release of synaptic Glu to reduce synaptic glutamatergic signaling

Examples of neurotransmitters and neurotransmitter receptors, and treatment substances that inhibit the function of receptors (“ITS”) and/or activate the function of receptors (“ATS”) are shown at Table 4.

TABLE 4
Neurotransmitters and Neurotransmitter Receptors

Neurotransmitter Agonist: Receptor [Receptor Type (Ion Channel Type Effected)]
Inhibitor Treatment Substance (“ITS”): Substance [Details (Info)] [Reference No]
Activator Treatment Substance (“ATS”): Substance [Details (Info)] [Reference No]
Glu + D-Ser/Gly: NMDA-R [Ionotropic (Na+/K+/Ca2+)]
ITS: Inhibitor: Scutellaria genus Preparation [Active: Baicalin (Decreases NMDA-R
Amount)]; Panax genus Preparation {e.g., A Panax ginseng/notoginseng/quinquefolius
Preparation [Active: Ginsenoside (e.g., Rb1/Rb2/Rc/Re/Rf/Rg1/Rg2/Rg3/Rh1/Rh2; Inhibits
NMDA-R′s Increase in Ca2+ in neurons)]}; Vitis vinifera Preparation [Active: Seed Extract
(Inhibits Ca2+ Signaling)]; Genistein; Daidzein; Glycine max Preparation [Active: Genistein];
Pueraria mirifica Preparation [Active: Genistein, Daidzein]; Hodgkinsine; Psychotridine;
Psychotria colorata Preparation [Active: Hodgkinsine; Psychotridine]; Theanine;
Rx{Bumetanide, Caroverine, Dexanabinol, Furosemide, Minocycline, Niflumic acid,
Pentamidine, Piretanide}; Antagonist: Crocus sativus Preparation [Active:
Transcrocetin/Crocetin/Crocin/Safranal]; Glycyrrhizae genus Preparation [Active:
Isoliquiritigenin]; Ethanol [Antagonist/NAM]; Rx{Kaitocephalin, Midafotel, Perzinfotel,
Chloroform, Nitrous oxide, Xenon}; Antagonist (Glycine Site): Kynurenic Acid; Kynurenine;
Rx{4-Chlorokynurenine, Apimostinel, Carisoprodol, D-Cycloserine, Gavestinel, Licostinel,
Meprobamate}; Antagonist (Polyamine Site): Agmatine; Huperzine A; Huperzia serrata
Preparation [Active: Huperzine A]; Putrescine; Inhibitor (e.g., Channel Blocker/Dizocilpine
Site Antagonist): Magnesium/Mg2+ [Channel Blocker; NMDA-R with GluN2B Subunit
NAM/Antagonist]; Zinc/Zn2+ [Weak Channel Blocker for NMDA with NR2A Subunit];
Spermidine/Spermine/Histamine [Channel Blocker for NMDA-R with NR2A/NR2B Subunit
But PAM when Gly Concentration Low]; Oryza sativa Preparation [Active: Spermidine];
Coronaridine; Dextromethorphan [Dextrorphan Prodrug (Dextrorphan a Channel Blocker);
Degradation Inhibited by Quinidine Sulfate for Greater Effect]; Dextrorphan; Ibogamine;
Isorhynchophylline; Rhynchophylline; Uncaria genus Preparation [e.g., Uncaria
tomentosa/rhynchophylla/guianensis Preparation; Active: Isorhynchophylline,
Rhynchophylline]; Sabeluzole; Tabernanthine; Rx{18-Methoxycoronaridine, Amantadine,
Arketamine, Budipine, Delucemine, Dextromethadone, Gacyclidine, Indantadol, Ketamine,
Ketobemidone, Lanicemine, Levomethadone, Levomilnacipran, Levorphanol, Loperamide,
Memantine, Methadone, Milnacipran, Neramexane, Nitromemantine, Noribogaine,
Norketamine, Orphenadrine, Remacemide, Rimantadine, Tiletamine, Tramadol }; Inhibitor
(e.g., Ifenprodil Site/Receptor with NR2B Subunit Antagonist): Rx{Besonprodil, Eliprodil,
Haloperidol, Isoxsuprine, Rislenemdaz, Traxoprodil}; NAM: Rx{Zelquistinel} [10, 12, 16, 17,
19, 21, 22, 44, 45, 49, 50, 71, 72, 105, 106, 107, 117, 119, 120, 123]
ATS: Activator: GSH [Reduces NMDA-R Oxidation and that Promotes NMDA-R Activity
and Neurotransmitter Release]; Phenylpiracetam [Injection increased NMDA-R Amount];
Agonist (Glu Site): NMDA; Asp; D-Asp; Cys; Pro; Theanine; Agonist (Gly Site): Ser;
Sarcosine; Betaine [Partial Agonist]; DimethylGly [Partial Agonist]; Ala; D-Ala; Milacemide
[Gly Prodrug]; Rx{Apimostinel, D-Cycloserine, Neboglamine}; Agonist (Polyamine Site):
Spermidine; Oryza sativa Preparation [Active: Spermidine]; Spermine; Rx{Neomycin}; PAM:
DHEA; DHEAS; Epipregnanolone Sulfate; Pregnenolone Sulfate; 24S-Hydroxycholesterol;
Uncarine E; Uncaria genus Preparation [Active: Uncarine E (Agonist/PAM)]; Modulator:
Acetyl-Carnitine [Modulates NMDA-R NR1 Subunit Amount] [12, 13, 14, 40, 44, 73, 74, 71,
72, 105, 106, 107, 109, 118]
Glu: AMPA-R [Ionotropic (Na+/K+/Ca2+)]
ITS: Inhibitor: Glu [Extrasynaptic Glu Reduces the postsynaptic neuron′s AMPA-R Amount
on the cell′s surface]; Vitis vinifera Preparation [Active: Seed Extract (Inhibits Ca2+
Signaling)]; Calcium/Ca2+ [Inactivates AMPA-R by Movement of AMPA-R from Cell
Surface]; Antagonist: Ethanol [Antagonist/NAM]; Capric Acid/Spermine/Spermidine [Channel
Blocker]; Oryza sativa Preparation [Active: Spermidine]; Theanine; Rx{Becampanel,
Caroverine, Dasolampanel, Kaitocephalin, Licostinel, Selurampanel, Tezampanel, Topiramate,
Minocycline}; NAM: Pregnenolone Sulfate; Kynurenic Acid; Kynurenine; Rx{Barbiturate
(e.g., Sodium Thiopental, Pentobarbital), Irampanel, Perampanel } [19, 21, 22, 24, 42, 45, 47];
ATS: Activator: Scutellaria genus Preparation [Active: Baicalin (Increases AMPA-R
amount)]; Zinc/Zn2+; Agonist: AMPA; Pro; Willardiine; Activator (AMPA-R Having a
GluA1 Subunit): Agmatine [AMPA-R Activiation Promotes BDNF Release]; PAM:
Pramiracetam; Aniracetam; Nooglutyl; Rx{Pesampator, Cyclothiazide, Ampalex, Diazoxide
Hydrochlorothiazide, Mibampator, Oxiracetam, Piracetam, Tulrampator} [119]
Glu: Kainate-R (“KA-R”) [Ionotropic (Na+/K+/Ca2+)]
ITS: Antagonist: Ethanol [Antagonist/NAM]; Theanine; Spermine/Spermidine [Channel
Blocker]; Oryza sativa Preparation [Active: Spermidine]; Rx{Dasolampanel,
Kaitocephalin, Licostinel, Selurampanel, Tezampanel, Topiramate}; NAM: Pregnenolone
Sulfate; Kynurenic Acid; Rx{Barbiturate (e.g., Pentobarbital)} [19, 21, 22, 45, 119]
ATS: Agonist: Kainate; AMPA; Pro; PAM: Rx{Cyclothiazide, Diazoxide}
Glu: mGlu1-R [Metabotropic]
ITS: Antagonist: Vitis vinifera Preparation [Active: Seed Extract]; Rx{Cyclothiazide}
ATS: Agonist: Theanine; Rx{Fasoracetam }
Glu: mGlu5-R [Metabotropic]
ITS: Antagonist: Rx{Basimglurant, Dipraglurant, Raseglurant, Mavoglurant, Remeglurant}
ATS: Agonist: D-Asp; Vitis vinifera Preparation [Active: Seed Extract]; Rx{Fasoracetam}
[40]
Glu: mGlu2-R [Metabotropic]
ITS: NAM: Rx{Decoglurant}
ATS: Activator: Acetyl-Carnitine [Increases mGlu2-R in the Cerebral Cortex/Spinal cord upon
days of ingestion]; Agonist: Rx{Fasoracetam, Biphenylindanone A, Pomaglumetad,
Eglumegad, Possibly Noopept} [118]
Glu: mGlu3-R [Metabotropic]
ITS: NAM: Rx{Decoglurant}
ATS: Agonist: Rx{Fasoracetam, Eglumegad, Pomaglumetad, Pomaglumetad Methionil}
Glu: mGlu4-R [Metabotropic]
ATS: Agonist: Rx{Fasoracetam }; PAM: Rx{Foliglurax}
Glu: mGlu7-R [Metabotropic]
ATS: Agonist: Rx{Fasoracetam }
Glu: mGlu8-R [Metabotropic]
ATS: Agonist: Ginkgo biloba Preparation; Rx{Fasoracetam}
Glu: mGlu6-R [Metabotropic]
ITS: Inhibitor: Spermine/Spermidine [Channel Blocker]; Oryza sativa Preparation [Active:
Spermidine] [119]
ATS: Agonist: Rx{Fasoracetam}
GABA: GABAA-R [Ionotropic (C1-, HCO3 -; Exchanger)]
ITS: Antagonist: Hydrastine; Sinomenine; Apigenin [Affects GABAA-R Having
Alphal/Betal/Gamma2S Subunit]; Matricaria chamomilla Preparation [Active: Apigenin];
Genistein; Daidzein; Glycine max Preparation [Active: Genistein]; Pueraria mirifica
Preparation [Active: Genistein, Daidzein]; NAM: DHEA; DHEAS; Bilobalide;
Amentoflavone; Ginkgo biloba Preparation [Active: Bilobalide]; Hypericum perforatum
Preparation [Active: Amentoflavone, Bilobalide]; Oroxylin A; Scutellaria genus Preparation
[Active: Oroxylin A]; Oroxylum indicum Preparation [Active: Oroxylin A]; Epipregnanolone;
Isopregnanolone; 11-Ketoprogesterone; 17-Phenylandrostenol; Pregnenolone Sulfate;
Zinc/Zn2+; Rx{Amiloride, Beta-Lactam (e.g., Cephalosporin, Carbapenem, Penicillin),
Basmisanil, Bemegride, Bicalutamide, Cyclothiazide, Furosemide, Flumazenil, Morphine,
Naloxone, Naltrexone, Nilutamide, Nicardipine, Bupropion; Ciprofloxacin, Apalutamide,
Enzalutamide, Laudanosine, Leptazol, Morphine, Morphine-3-Glucuronide}; NAM
(Benzodiazepine Site Antagonist/Inverse Agonist): Rx{Iomazenil (Partial Inverse
Agonist/Antagonist), Radequinil (Partial Inverse Agonist), Suritozole (Inverse Agonist),
Terbequinil (Partial Inverse Agonist)}; NAM (Neurosteroid Site Antagonist):
Rx{Golexanolone (Antagonist for Neurosteroids such as Allopregnanolone
(“APL”)/Tetrahydrodeoxycorticosterone)}; NAM (Alcohol Site Antagonist):
Dihydromyricetin; Rx{Thujone} [1, 61, 62, 63, 122]
ATS: Agonist: Homotaurine [Partial Agonist]; Phenibut [Stronger as a GABAB-R Agonist];
Beta-Ala; Taurine; Catechin [Agonist/PAM]; Borneol; Blumea balsamifera
Preparation/Kaempferia galanga Preparation [Active: Borneol (Agonist/PAM)]; Withanolide
[e.g., Withaferin A]; Withania somnifera Preparation [Active: Withanolide (e.g., Withaferin A)
(Agonist/PAM)]; Valeriana officinalis Preparation [Active: Valerenic Acid (GABAA-R with
Beta3 Subunit Agonist)]; Nefiracetam; Quisqualamine; Thiomuscimol; Gastrodia elata
Preparation [Active: Gastrol, Gastrodin, Bis(4-hydroxybenzyl)sulfide,
N6-(4-hydroxybenzyl)adenine Riboside (Possible Agonist)]; Passiflora genus Preparation
[Active: Chrysin (Benzodizepine Site PAM; Agonist/Antagonist)]; Rx{Bamaluzole,
Barbiturates (e.g., Phenobarbital), Gaboxadol, Isonipecotic Acid (Partial Agonist), Muscimol };
GABA Prodrug (Metabolized into GABA): NGABA [Likely Hydrolyzed into GABA and
Niacin]; Isonicotinoyl-GABA; Pyridoxalphosphate-GABA; Rx{Progabide, Progabide Acid,
Beta-Hydroxy-GABA (PAM/Agonist), Isoguvacine (Agonist), Tolgabide}; PAM: Niacin,
Nicotinamide/Niacinamide; 4-O-Methylhonokiol/Obovatol; Honokiol/Magnolol; Magnolia
genus Preparation [e.g., Magnolia grandiflora/obovate/virginiana Preparation; Active:
4-O-Methylhonokiol/Obovatol (Benzodiazepine Site PAM; Increases Alphal Subunit Amount);
Honokiol/Magnolol (PAM at Different Site than Neurosteroid/Anesthetic/Ethanol/Picrotoxin
Sites)]; Thymol; Eugenol; Syzygium aromaticum Preparation [Active: Thymol, Eugenol];
Carvacrol; Alpha-Pinene; Rosmarinus officinalis Preparation/Satureja myrtifolia Preparation
[Active: Alpha-Pinene]; Menthol; Epigallocatechin Gallate; Hispidulin; Linarin; Luteolin;
Arachis hypogaea Preparation [Active: Luteolin]; Desmethoxyyangonin; Kavain;
Dihydrokavain; Methysticin; Yangonin; Piper methysticum Preparation [Active: Kavalactone
(e.g., Desmethoxyyangonin, Kavain, Dihydrokavain, Methysticin, Yangonin) (Not
Benzodiazepine Site PAM)]; Loreclezole; Etaqualone [Agonist for GABAA-R with Beta
Subunit]; Lavandula genus Preparation [(e.g., Lavandula angustifolia/latifolia Preparation);
Active: Linalool, Linayl Acetate]; Rx{Zolpidem, Petrichloral, Avermectin (e.g, Ivermectin),
Phenobarbital, Meprobamate, Carisoprodol, N-pantoyl-GABA, Chloroform, Chloral Hydrate,
Dichloralphenazone, Nitrous Oxide, Carbamazepine, Ergoline (e.g., Dihydroergocryptine,
Dihydroergotamine), Mefenamic Acid, Niflumic Acid, Tolfenamic Acid, Fluoxetine, Menthyl
Isovalerate, Phenytoin, Topiramate, Carbamate, Imidazole (e.g., Etomidate), Acylurea (e.g.,
Apronal, Bromisoval)}; PAM (Benzodiazepine Site): Magnesium/Mg2+, Crocus sativus
Preparation [Active: Safranal, Crocetin, Dimethylcrocetin (Agonist/PAM)]; Methylapigenin;
Apigenin; Valeriana wallichii Preparation [Active: 6-Methylapigenin (Agonist/PAM)];
Tanacetum parthenium Preparation [Active: Apigenin]; Matricaria chamomilla Preparation
[Active: Apigenin]; Baicalein; Baicalin; Wogonin; Scutellaria genus Preparation [Active:
Baicalin, Baicalein, Wogonin]; Valerenol; Valerenic Acid; Valeric Acid; Isovaleric Acid;
Valeriana officinalis Preparation [Active: Valerenol, Valerenic Acid, Valeric Acid, Isovaleric
Acid]; Glycyrrhizae genus Preparation [Active: Isoliquiritigenin, Glabrol]; Rx{Benzodiazepine
(e.g., Bromazepam, Clonazepam, Diazepam, Alprazolam), Nonbenzodiazepine (e.g.,
Pazinaclone, Zopiclone, Alpidem, Zolpidem, Indiplon, Zaleplon, Lirequinil, Viqualine),
Tracazolate (PAM for GABAA-R with Alphal/Beta3 Subunit)}; PAM (Non-Benzodiazepine
Site): Dehydroabietic Acid; Incensole; Incensole Acetate; Boswellia serrata Preparation
[Active: Dehydroabietic Acid, Incensole, Incensole Acetate]; PAM (Neurosteroid Site):
Allotetrahydrodeoxycorticosterone; Pregnanolone; Androstenol; Androsterone; Cholesterol;
5 Alpha-Dihydrodeoxycorticosterone; 3 Alpha-Dihydroprogesterone;
5 Alpha-Dihydroprogesterone; 5Beta-Dihydroprogesterone; Dihydrotestosterone;
Etiocholanolone; Rx{Allopregnanolone, 3 Alpha-Androstanediol, Acebrochol, Alfadolone,
Alfaxalone, Ganaxolone, Hydroxydione, Posovolone, Progesterone, Renanolone, Testosterone,
Zuranolone}; PAM (Barbiturate Site): Rx{Etazolate, Clomethiazole, Barbituate (e.g.,
Pentobarbital, Sodium Thiopental)}; PAM (Ethanol Site): Ethanol [1, 18, 19, 21, 22, 26, 30,
34, 26-39, 44, 45, 61, 62, 63, 67, 102, 110, 114, 124]
GABA: GABAARho-R [Ionotropic (Cl&emdash;, HCO3&emdash; Exchanger)]
ITS: Antagonist: Apigenin; Matricaria chamomilla Preparation [Active: Apigenin];
(1,2,5,6-Tetrahydropyridin-4-yl)methylphosphinic Acid; Isonipecotic Acid; Loreclezole
[Antagonist/NAM]; Rx{Gaboxadol}; NAM: Bilobalide; Ginkgo biloba Preparation [Active:
Bilobalide]; Hypericum perforatum Preparation [Active: Bilobalide];
5 Alpha-Dihydroprogesterone; Pregnanolone; THDOC; Zinc/Zn2+; Ethanol [NAM for some
GABA-R having Rhol Subunit] [1, 19, 22, 61, 62, 63]
ATS: Agonist: Thiomuscimol; Withania somnifera Preparation [Active: Withanolide (e.g.,
Withaferin A), Triethylene Glycol (Agonist/PAM)]; Beta-Ala; Taurine; Rx{Muscimol}; GABA
Prodrug (Metabolized into GABA): [See GABAA-R Above]; PAM (Neurosteroid site):
THDOC; Rx{Allopregnanolone, Alfaxalone}; PAM (Ethanol Site): Ethanol [1, 45, 102, 115,
124]
GABA: GABAB-R [Metabotropic]
ITS: Antagonist: Homotaurine [Partial Antagonist/Partial Agonist]; Rx{Saclofen, Phaclofen}
[1, 26, 27-39]
ATS: Agonist: Phenibut (Full Agonist); GHB [Weak Agonist]; Passiflora genus Preparation
[Active: Chrysin (GABAB-R Agonist/Antagonist)]; Taurine [Possible Agonist]; Rx{Baclofen,
Arbaclofen, Arbaclofen Placarbil, Tolibut, 4-Fluorophenibut, Aceburic Acid, Lesogaberan,
Sodium Oxybate; Fasoracetam (Increases GABAB-R Amount)}; GABA Prodrug
(Metabolized into GABA): [See GABAA-R Above] [1, 26, 27-39, 45, 124]
Gly: Gly-R [Ionotropic (Cl—)]
ITS: Antagonist: Caffeine; Coffea arabica Preparation/Coffea robusta Preparation [Active:
Caffeine]; Cannabinoid [e.g., 2-AG, Anandamide]; Isobutyric Acid; Sinomenine;
Rx{Colchicine, Gaboxadol, Isonipecotic Acid, Laudanosine}; NAM: Daidzein; Genistein;
Glycine max Preparation [Active: Genistein]; Pueraria mirifica Preparation [Active: Daidzein,
Genistein]; Bilobalide; Ginkgo biloba Preparation [Active: Bilobalide, Ginkgolide (e.g.,
Ginkgolide A/B/C/J/M)]; Neurosteroid [e.g., 11-Deoxycorticosterone, DHEAS, Pregnenolone
Sulfate]; Dextromethorphan; Dextrorphan; Zinc/Zn2+ [High Concentration]; Rx{Amiloride,
Benzodiazepine (e.g., Bromazepam, Clonazepam, Flurazepam), Diazepam, Nicardipine,
Nitrendipine, Furosemide, Imipramine, Levomethadone, Levorphanol, Morphine, Progesterone,
Codeine, Riluzole, Tropisetron, Zatosetron, Verapamil} [23]
ATS: Agonist: Beta-Ala; D-Ala; Ala; Beta-Aminobutryic Acid; GABA; D-Ser; Ser; Pro; Thr;
Sarcosine; Taurine; Rx{Ivermectin (Agonist/PAM)}; Gly Prodrug (Metabolized into Gly):
Milacemide; PAM: Glu; Anandamide; Panax genus Preparation [Active: Ginsenoside (e.g.,
Ginsenoside-Rf)]; Atropine; Atropa belladonna Preparation [Active: Atropine]; Pregnenolone;
Zinc/Zn2+; Ethanol; Rx{Barbituate (e.g., Pentobarbital, Sodium Thiopental), Nicardipine,
Etomidate, Ketamine, Nitrous Oxide, Tetrahydrocannabinol, Tropisetron, Zatosetron, Chloral
Hydrate, Xenon} [19, 20, 23, 105]
Acetylcholine: nACh-R (“Nicotinic Acetylcholine-R,” “Nicotinic ACh-R”) [Ionotropic
(Na+, K+, Ca2+)]
ITS: Antagonist: Coclaurine; Coronaridine; Erythravine; Kynurenic Acid; Lobeline [Possible
Antagonist of nACh-R having Alpha7 Subunit/Alpha4/Beta2 Subunit]; Lobelia inflata
Preparation [Active: Lobeline]; Rx{Barbituate (e.g., Pentobarbital, Sodium Thiopental),
Bupropion, Ketamine, Laudanosine, Levomethadone, Mecamylamine, Memantine, Methadone,
Neramexane, Nitrous Oxide, Norketamine, Pempidine, Progesterone, Reboxetine, Tramadol,
Xenon}; Antagonist (nACh-R with Alpha3/Beta4 Subunit): Dextromethorphan/Dextrorphan
[Possible Antagonist/NAM]; Rx{18-Methoxycoronaridine}; Antagonist (nACh-R with Alpha
7 Subunit): Rx{Amantadine, Encenicline (Partial Agonist/Antagonist)}; NAM:
Rx{Allopregnanolone}; NAM (nACh-R with Alpha 7 Subunit): Rx{Hydroxynorketamine,
Dehydronorketamine} [10, 11, 12, 120]
ATS: Activator: Phenylpiracetam [Injection increased nACh-R Amount]; Agonist: Anatabine;
Butyrylcholine; Desformylflustrabromine [Agonist/PAM]; Ethanol [Agonist/PAM]; Uncarine
E; Rhynchophylline; Uncaria genus Preparation [Active: Uncarine E, Rhynchophylline
(Agonist/PAM); Active: Rhynchophylline (nACh-R with Alpha3/Beta4 Subunit
Agonist/PAM)]; Agmatine; Epigallocatechin Gallate; Camellia sinensis Preparation [Active:
Epigallocatechin Gallate]; Lobeline [Agonist/Possible Antagonist for nACh-R with
Alpha4/Beta2/Alpha7 Subunit]; Lobelia inflata Preparation [Active: Lobeline]; Rx{Ivermectin,
Cotinine, Cytisine (Partial Agonist), Nicotine (Agonist nACh-R, but Antagonist for nACh-R
having Alpha9/Alpha10 Subunit), Dimethylphenylpiperazinium (Agonist for Ganglion
nACh-R)}; Agonist (nACh-R having Alpha4/Beta2 Subunit): Rx{Altinicline, Butinoline,
Dianicline (Partial Agonist), Pozanicline, Ispronicline (Partial Agonist), Rivanicline (Partial
Agonist), Varenicline (Partial Agonist), Sazetidine A (Agonist for nACh-R having Two Alpha4
Subunit; Antagonist for nACh-R having Three Alpha4 Subunit)}; Agonist (nACh-R having
Alpha7 Subunit): Choline; Nigella sativa Preparation [Active: Thymoquinone];
Rx{Tropisetron}; PAM: Galantamine; Galantamine Hydrobromide; Lycoris radiatia
Preparation [Active: Galantamine Hydrobromide]; [10, 12, 13, 14, 22, 45, 48, 51, 105, 106,
107, 118]
Acetylcholine: mACh-R (“Muscarinic Acetylcholine-R,” “Muscarinic ACh-R”)
[Metabotropic]
ITS: Antagonist: Atropine; Atropa belladonna Preparation [Active: Atropine, Scopolamine];
Rhynchophylline; Isorhynchophylline; Uncaria genus Preparation [Active:
Rhynchophylline/Isorhynchophylline (Antagonist/NAM)]; Cinchona officinalis Preparation
[Active: Quinidine]; Rx{Antihistamine (e.g., Brompheniramine, Buclizine, Chlorpheniramine,
Diphenhydramine, Doxylamine, Meclizine), Atropine Methonitrate, Atypical Antipsychotic
(e.g., Quetiapine), Benzatropine, Biperiden, Camylofin, Caramiphen, Etybenzatropine,
Orphenadrine, Quinidine, SSRI (e.g., Paroxetine), Tetracyclic Antidepressants (e.g.,
Amoxapine, Maprotiline), Tricyclic Antidepressant (e.g., Amitriptylinexide, Quinupramine),
Typical Antipsychotic (e.g., Chlorprothixene)}; NAM: Ethanol [22, 48, 105, 106, 107 116]
ATS: Agonist: Choline; Butyrylcholine; Uncarine E; Uncarine C; Mitraphylline; Uncaria
genus Preparation [Active: Oxindole Alkaloids (e.g., Uncarine E, Uncarine C, Mitraphylline)
(Agonist/PAM)]; Rx{Aceclidine, Bethanechol, Bevonium, Cevimeline, Methacholine,
Oxotremorine, Pilocarpine, Vedaclidine} [105, 106, 107]
Dopamine: D1-R/D5-R [Metabotropic]
ITS: Antagonist (D1-R/D5-R): Rx{Typical Antipsychotic (e.g., Chlorprothixene), Atypical
Antipsychotic (e.g., Risperidone, Ziprasidone)}
ATS: Agonist (D1-R/D5-R): Phe; Tyr; Corynanthine; Rauwolscine; Yohimbine [Mixture of
Rauwolscine and Corynanthine (DIA-R Partial Agonist)]; Corynanthe johimbe
Preparation/Pausinystalia yohimbe Preparation [Active: Yohimbine]; Rauwolfia vomitoria
Preparation [Active: Rauwolscine]; DOPA; Tetrahydropalmatine; Rx{Fenoldopam (Partial
Agonist), Dihydroergocryptine, Dihydrexidine, Dinapsoline (D1-R Agonist), Ibopamine (D1-R
Agonist), Melevodopa (DOPA Prodrug)}
Dopamine: D2-R/D3-R/D4-R [Metabotropic]
ITS: Antagonist (D2-R/D3-R/D4-R): Corynanthine; Rauwolscine; Yohimbine; Corynanthe
johimbe Preparation/Pausinystalia yohimbe Preparation [Active: Yohimbine]; Rauwolfia
vomitoria Preparation [Active: Rauwolscine]; Tetrahydropalmatine; Rx{Typical Antipsychotic
[e.g., Chlorprothixene, Haloperidol, Levosulpiride (D2-R Antagonist), Nemonapride
(D2-R/D3-R Antagonist)], Atypical Antipsychotic [Melperone (D2-R Antagonist),
Mosapramine (D2-R/D3-R/D4-R Antagonist), Risperidone, Ziprasidone], Amoxapine,
Buspirone, Fananserin (D4-R Antagonist)}
ATS: Agonist (D2-R/D3-R/D4-R): DOPA; Phe; Tyr; Rx{Amantadine, Memantine,
Rimantadine, Cabergoline (D2-R Agonist), Dihydroergocryptine, Melevodopa (DOPA
Prodrug), Aplindore (D2-Partial Agonist), Arketamine, Armodafinil, Flibanserin, Ketamine,
Modafinil, Piribedil (D2-R/D3-R Agonist), Pramipexole, Ropinirole, Roxindole}
Adenosine: A1-R/A2A-R/A2B-R/A3-R [Metabotropic]
ITS: Antagonist: Caffeine; Coffea arabica Preparation/Coffea robusta Preparation [Active:
Caffeine]; Theobromine; Theobroma cacao Preparation [Active: Theobromine] [8, 101]
Adenosine: A1-R [Metabotropic]
ATS: Agonist: Incarvillea sinensis Preparation [Active: Incarvillateine] [99, 100]
Adenosine: A2A-R [Metabotropic]
ATS: Gastrodia elata Preparation [Active: N6-(4-hydroxybenzyl)adenine riboside)] [128]
Adenosine: A3-R [Metabotropic]
ATS: Agonist: Inosine
Histamine: H1-R [Metabotropic]
ITS: Antagonist: Meclizine; Diphenhydramine [Inverse Agonist]; Rx{Benzatropine,
Brompheniramine, Buclizine, Chlorpheniramine (Inverse Agonist), Doxylamine,
Etybenzatropine, Orphenadrine, Atypical Antipsychotic (e.g., Risperidone, Ziprasidone),
Amoxapine, Desipramine, Imipramine}
ATS: Agonist: His; Rx{Betahistine}
Histamine: H2-R [Metabotropic]
ITS: Antagonist: Rx{Cimetidine, Famotidine} [58]
ATS: Agonist: His
Histamine: H3-R [Metabotropic]
ITS: Inhibitor: Conessine; Inverse Agonist: Rx{Pitolisant}; Antagonist: Rx{Betahistine,
Thioperamide} [6, 112]
ATS: Agonist: His
Histamine: H4-R [Metabotropic]
ITS: Antagonist: Rx{Thioperamide, Toreforant}
ATS: Agonist: His
Neurosteroid: Sigma1-R (“Sigmal Receptor”) [Metabotropic]
ATS: Activator: DHEA; DHEAS; Pregnenolone; Agonist: Rx{Dextromethorphan,
Pridopidine} [120]
Adrenaline/Noradrenaline: Alpha1-R [Metabotropic]
ITS: Antagonist: Delta-Yohimbine; Raubasine; Corynanthine [Stonger Antagonist than
Rauwolscine]; Rauwolscine; Yohimbine; Corynanthe johimbe Preparation/Pausinystalia
yohimbe Preparation [Active: Yohimbine]; Rauwolfia vomitoria Preparation [Active:
Rauwolscine]; Rx{Ajmalicine, Risperidone, Carvedilol, Ergotamine, Phentolamine,
Amoxapine, Imipramine}
ATS: Agonist: DOPA; Dopamine; Phe; Tyr; Synephrine [Agonist/Antagonist]; Rx{Buspirone,
Droxidopa (Noradrenaline Prodrug), Melevodopa (DOPA Prodrug), Metaraminol, Methyldopa,
Octopamine, Oxymetazoline, Phenylephrine, Xylometazoline} [113]
Adrenaline/Noradrenaline: Alpha2-R [Metabotropic]
ITS: Antagonist: Yohimbine; Corynanthine; Rauwolscine [Stronger Antagonist than
Corynanthine]; Corynanthe johimbe Preparation/Pausinystalia yohimbe Preparation [Active:
Yohimbine]; Rauwolfia vomitoria Preparation [Active: Rauwolscine]; Rx{Risperidone,
Buspirone, Piribedil} [11, 43, 105]
ATS: Agonist: Dopamine; Agmatine; Phe; Tyr; DOPA; Rx{Apraclonidine, Brimonidine,
Clonidine, Corbadrine, Droxidopa (Noradrenaline Prodrug), Ergotamine, Dihydroergotamine,
Guanabenz, Guanfacine, Lofexidine, Melevodopa (DOPA Prodrug), Methyldopa,
Oxymetazoline, Phenylpropanolamine, Xylometazoline} [12, 13, 14]
Adrenaline/Noradrenaline: Beta1-R [Metabotropic]
ITS: Inhibitor: Piper methysticum Preparation [Active: Kavain, Dihydrokavain (Reduces
Betal-R Amount)]; Hypericum perforatum Preparation [Reduces Betal-R Amount];
Antagonist: Rx{Acebutolol, Arotinolol, Atenolol, Betaxolol, Bisoprolol, Carteolol, Carvedilol,
Dilevalol, Labetalol, Nadolol, Nebivolol, Oxprenolol, Pindolol, Timolol} [102, 103]
ATS: Agonist: Higenamine; DOPA; Dopamine; Phe; Tyr; Rx{Abediterol, Arotinolol,
Clenbuterol, Dobutamine, Droxidopa (Noradrenaline Prodrug), Formoterol, Isoetarine,
Isoprenaline, Isoxsuprine, Levosalbutamol, Melevodopa (DOPA Prodrug), Methyldopa,
Phenylpropanolamine, Vilanterol }
Serotonin: 5HT1-R (e.g., 5HTIA-R, 5HT1B-R) [Metabotropic]
ITS: Antagonist (5HT1A-R): Rx{Risperidone, Carvedilol}; Antagonist (5HT1B-R):
Yohimbine; Corynanthe johimbe Preparation/Pausinystalia yohimbe Preparation [Active:
Yohimbine]; Rx{Cabergoline, Carteolol, Oxprenolol, Penbutolol}; Antagonist (5HT1D-R):
Yohimbine; Corynanthe johimbe Preparation/Pausinystalia yohimbe Preparation [Active:
Yohimbine]; Rx{Ketanserin, Ziprasidone}
ATS: Activator (5HT1A-R): Hypericum perforatum Preparation [Increases Postsynaptic
Neuron′s 5HT1A-R Amount]; Agonist: (5HT1A-R): Dopamine; Yohimbine; Rauwolscine;
Corynanthe johimbe Preparation/Pausinystalia yohimbe Preparation [Active: Yohimbine];
Rauwolfia vomitoria Preparation [Active: Rauwolscine]; Rx{Cabergoline, Ergotamine,
Flibanserin, Roxindole, Ziprasidone}; Agonist (5HT1B-R): Rx{Ergotamine}; Agonist
(5HT1D-R): Rx{Cabergoline, Ergotamine} [103]
Serotonin: 5HT2-R (e.g., 5HT2A-R, 5HT2B-R, 5HT2C-R) [Metabotropic]
ITS: Inhibitor: Rhynchophylline; Isorhynchophylline; Uncaria genus Preparation [Active:
Rhynchophylline/Isorhynchophylline (5HT2-R Antagonist/NAM)]; Antagonist (5HT2A-R):
Yohimbine; Rauwolscine; Corynanthe johimbe Preparation/Pausinystalia yohimbe Preparation
[Active: Yohimbine]; Rauwolfia vomitoria Preparation [Active: Rauwolscine];
Rx{Deramciclane, Fananserin, Flibanserin, Ergotamine, Risperidone, Ziprasidone, Ketanserin};
Antagonist (5HT2B-R): Yohimbine; Rauwolscine; Corynanthe johimbe
Preparation/Pausinystalia yohimbe Preparation [Active: Yohimbine]; Rauwolfia vomitoria
Preparation [Active: Rauwolscine]; Rx{Risperidone, Ziprasidone, Ergotamine, Ketanserin};
Antagonist (5HT2C-R): Rx{Risperidone, Ziprasidone, Ergotamine, Deramciclane,
Ketanserin} [105, 106, 107]
ATS: Activator (5HT2-R): Hypericum perforatum Preparation [Increases Postsynaptic
Neuron′s 5HT2-R Amount]; Agonist (5HT2A-R): Agmatine; Agonist
(5HT2A-R/5HT2B-R/5HT2C-R): Rx{Cabergoline}; Agonist (5HT2B-R):
Rx{Dihydroergocryptine} [12, 13, 14, 103, 105, 106, 107]
Serotonin: 5HT3-R [Ionotropic (Na+, K+)]
ITS: Antagonist: Rx{Bupropion, Memantine, Thujone, Tropisetron}; NAM: Anandamide [3]
ATS: Agonist: Ethanol [Agonist/PAM]; Agmatine [12, 13, 14, 22, 45]
Serotonin: 5HT4-R/5HT5-R (e.g., 5HT5A-R)/5HT6-R/5HT7-R) [Metabotropic]
ITS: Antagonist (5HT4-R): Lys; Antagonist (5HT6-R): Rx{Ketanserin}; Antagonist
(5HT7-R): Rx{Risperidone, Ziprasidone, Cabergoline, Ergotamine, Ketanserin}
ATS: Agonist (5HT5A-R): Valerenic Acid; Valeriana officinalis Preparation [Active:
Valerenic Acid (Partial Agonist)]; Rx{Ergotamine}; Agonist (5HT6-R): Rx{Ergotamine}
Melatonin: Melatonin 1A Receptor/Melatonin 1B Receptor [Metabotropic]
ITS: Antagonist: Rx{Luzindole}
ATS: Agonist: Rx{Agomelatine, Piromelatine, Ramelteon, Tasimelteon}
Cannabinoid: CB1-R [Metabotropic]
ITS: Antagonist: Virodhamine; NAM: Rx{Cannabidiol} [4, 56, 57]
ATS: Agonist: 2-AG; Anandamide [Partial Agonist]; Oleamide; Serinolamide A; Yangonin;
Piper methysticum Preparation [Active: Yangonin]; Rx{Nabilone, Tetrahydrocannabinol,
Cannabidiol (Partial Agonist)} [3, 52, 60]
Cannabinoid: CB2-R [Metabotropic]
ITS: Inverse Agonist: 4-O-Methylhonokiol [Inverse Agonist]; Magnolia genus Preparation
[Active: 4-O-Methylhonokiol] [121]
ATS: Agonist: 2-AG; Anandamide; Serinolamide A; Virodhamine; Beta-Caryophyllene;
Syzygium aromaticum Preparation [Active: Beta-Caryophyllene]; Piper nigrum Preparation
[Active: Beta-Caryophyllene]; 4-O-Methylhonokiol [Increases Intracellular Calcium];
Magnolia genus Preparation [Active: 4-O-Methylhonokiol (Partial Agonist)] [4, 52, 56, 57, 64,
65, 66, 121]
Cannabinoid: TRPV1-R [Ionotropic (Ca2+, Na+)]
ITS: DHEAS; DHEA [Competitive Inhibitor]; Capsaicin [Prolonged/Extreme Exposure
Reduces TRPV1-R Activity]; Rx{Mavatrep, Niflumic Acid, Tolfenamic Acid}
ATS: Agonist/Activator: Capsaicin (Agonist); Homocapsaicin; Homodihydrocapsaicin;
Nonivamide; Nordihydrocapsaicin; Capsicum genus Preparation [Active: Capsaicin,
Homocapsaicin, Homodihydrocapsaicin, Nonivamide, Nordihydrocapsaicin]; Anandamide;
Piperine [Agonist]; Piper longum Preparation/Piper nigrum Preparation [Active: Piperine];
Acetaminophen [Acetaminophen is metabolized with Arachidonic Acid into
N-arachidonoylphenolamine, and N-arachidonoylphenolamine a TRPV1-R Agonist and
Cannabinoid (e.g., Anandamide) Reuptake Inhibitor]; Spermine/Spermidine/Putrescine [At
High Concentrations]; Oryza sativa Preparation [Active: Spermidine]; Zingiber officinale
Preparation/Aframomum melegueta Preparation [Active: Hydroxy-Alpha-Sanshool (Possible
Agonist)]; Evodiamine; Tetradium genus Preparation [Active: Evodiamine]; Polyunsaturated
Fatty Acid [e.g., Arachidonic Acid (Metabolized Into the Agonist Hepoxilin A3/B3)];
Incensole; Vanillin (Agonist); Vanilla planifolia Preparation [Active: Vanillin]; Menthol;
Rx{Tramadol, Zucapsaicin, Cannabidiol} [3, 52, 53, 54, 55, 60]
Noradrenaline/Epinephrine/Imidazoline: I1-R/12-R [Metabotropic]
ATS: Agonist: Agmatine [12, 13, 14]
GHB: GHB-R [Metabotropic]
ATS: Agonist: Rx{Aceburic Acid, Gamma-Valerolactone, Sodium Oxybate}; PAM: Catechin
Opioid: Mu-R/Kappa-R [Metabotropic]
ITS: Antagonist: 4′,7-Dihydroxyflavone; Rx{Noribogaine}; Antagonist (Mu-R): Lobeline;
Lobelia inflata Preparation [Active: Lobeline]
ATS: Agonist: Incarvillea sinensis Preparation [Active: Incarvillateine]
1) Wolf, O. T. and Kirschbaum C. Brain Res. Brain Res. Rev. 1999 30(3): 264-288; 2) Ref. 53, Table 2; 3) Pertwee, R. G. Handb Exp Pharmacol. 2005 168: 1-51A; 4) De Petrocellis, L. et al. Br J Pharmacol 2004 141(5): 765-774; 5) Pertwee, R. G. Life Sci 2005 76(12): 1307-1324B; 6) Ref. 109, Table 2; 7) Shan, L. et al. Nat Rev Neurol. 2015 11(7): 401-413; 8) Masino, S. A. et al. Curr Neuropharmacol. 2009 7(3): 257-268; 9) Masino, S. A. et al. Epilepsy Res. 2012 100(3): 229-238; 10) Perry, E. and Howes, M.-J. R. CNS Neurosci. Ther. 2011 17: 683-698; 11) Li, J.-X. Pharmacol Ther 2017 178: 48-56; 12) Neis, V.B. et al. Neurochem Int. 2017 108: 318-331; 13) Freitas, A. E. et al. Eur Neuropsychopharmacol 2016 26(12): 1885-1899; 14) Kotagale, N. R. et al. Neurotoxicology 2019 73: 228-245; 15) Reiner, A. and Levitz, J. Neuron 2018 98: 1080-1098; 16) Ronald, K. M. et al. J. Biol. Chem. 2001 276: 44729-44735; 17) Ref. 122, Table 2; 18) Ref. 145, Table 2; 19) Perkins, D. I. et al. Pharmacol Ther. 2010 127(1): 53-65; 20) McCracken, L. M. et al. Neuropharmacology 2010 58: 676-681; 21) Ref. 146, Table 2; 22) Ref. 147, Table 2; 23) Burgos, C. F. et al. Mol Pharmacol 2016 90(3): 318-325; 24) Rogawski, M. A. Brain 2016 139(Pt 2): 306-309; 25) Ref. 110, Table 2; 26) Shaye, H. et al. Sci Adv. 2021 7(22): eabg3362; 27) Evenseth, L. S. M. et al. Molecules 2020 25(13): 3093; 28) Ref. 170, Table 2; 29) Cartmell, J. and Schoepp, D. D. J Neurochem 2000 75: 889-907; 30) Ren, S.-Y. et al. Acta Pharmacol Sin. 2020 41(10): 1263-1271; 31) Reddy, D. S. Front Cell Neurosci. 2013 7: 115; 32) Ref. 163, Table 2; 33) Ref. 164, Table 2; 34) Bridges, R. et al. Neurochem. 2015 135(6): 1062-1079; 35) Farant, M. and Nusser, Z. Nature Reviews Neuroscience 2005, 6(3): 215-219; 36) Ref. 166, Table 2; 37) Handbook of Clinical Neurology, Vol. 83, Parkinson′s Disease and Related Disorders Part I Koller, W. C. and Melamed, E. Eds 2007 Elsevoer Science; 38) Ranjbar-Slamloo, Y. and Fazlali, Z. Front Mol Neurosci 2020 12: 334; 39) Reiderer, P. and Muller, T. Expert Opin Drug Metab Toxicol 2017 13(2): 233-240; 40) Dalangin, R. et al. Int J Mol Sci. 2020 21(17): 6197; 41) Johnson, K. A. and Lovinger, D. M. Adv Pharmacol 2020 88: 193-232; 42) Ref. 165, Table 2; 43) Raasch, W. et al. Br J. Pharmacol. 2001 133: 755-780; 44) Tsuchiya, H. Molecules 2017 22(8): 1369; 45) Elmenhorst, E.-M. et al. Proc Natl Acad Sci USA 2018 115(31): 8009-8014; 46) Maciejak, P. et al. Neuroscience. 2016 313: 130-148; 47) Chang, P. et al. Brain 2016 139(Pt 2): 431-443; 48) Ref. 8, Table 3; 49) Papouin, T, et al. Cell. 2012 150(3): 633-646; 50) Panatier, A. et al. Cell 2006 125, 775-784; 51) Papke, R. L. and Lindstrom, J. M. Neuropharmacology 2020 168: 108021; 52) Raduner, S. et al. J Biol Chem 2006 281: 14192-14206; 53) Hogestatt, E. D. et al. 2005 J Biol Chem. 280(36): 31405-31412; 54) Bertolini, A. et al. CNS Drug Rev. 2006 12(3-4): 250-275; 55) Pertwee, R. G. Int J Obes (Lond). 2006 30 Suppl 1: S13-S18; 56) An, D. et al. Int J Mol Sci 2020 21(14): 5064; 57) Shahbazi, F. et al. iScience. 2020 23(7): 101301; 58) Ref. 45, Table 2; 59) Wong, C. G. T. et al. Toxicol Rev 2004 23(1): 3-20; 60) Zou, S. and Kumar, U. Int J Mol Sci 2018 19(3): 833; 61) Jager, A. K. et al. Phytother. Res. 2009 23: 1642-1644; 62) Campbell, E. L. et al. Biochem. Pharmacol. 2004 68: 1631-1638; 63) Gazola, A. C. et al. Rev. Bras. Farm. 2015 25: 158-163; 64) Bisogno, T. and Maccarrone, M. Expert Opin. Drug Discov. 2013 8(5): 509 522; 65) Gertsch, J. et al. Proc. Natl. Acad. Sci. USA 2008 105: 9099-9104; 66) Klauke, A. L. et al. Eur. Neuropsychopharmacol. 2014 24: 608-620; 67) Cho, S. et al. Bioorg. Med. Chem. 2012 20: 3493-3501; 68) Dorofeev, B. F. and Kholodov, L. E. Farmakol Toksikol 1991 54(2): 66-69; 69) Matsuyama, K. et al. Chem Pharm Bull (Tokyo) 1984 32(10)4089-4095; 70) Karaev, A. L. et al. Farmakol Toksikol 1989 52(1): 56-58; 71) Zhang, J. M. and Hu, G. Y. Neuroscience 2001 105(3): 663-669; 72) Rock, D. M. and MacDonald, R. L. Mol Pharmacol 1992 42(1)157-164; 73) Zyablitseva, E. A. et al. Span J Psycol 2009 12(1): 12-60; 74) Lee, M.-Y. et al. J Biomed Sci 2017 24(1): 18; 75) Chen, S. T. et al. Biomed Pharmacother. 2021 144: 112369; 76) Forte, N. et al. Epilepsia. 2016 57(12): 1987-2000; 77) Barbaccia, M. L. et al. Br. J. Pharmacol. 1997 120: 1582-1588; 78) Chaki, S. Neurosci Lett 2006 404: 182-186; 79) Wilkie, C. M. et al. Front Cell Neurosci. 2021 15: 661412; 80) Miller, B. R. et al. Neuroscience 2008 153: 329-337; 81) De Bundel, D. et al. J Neurosci. 2011 31: 5792-5803; 82) Baker, D. A. et al. J Neurosci 2002 22(20): 9134-9141; 83) Ahern, G. P. et al. J. Biol. Chem. 2006 281(13)8991-8995; 84) Williams, K. Cell Signal. 1997 9(1): 1-13; 85) Fleidervish, I.A. et al. Proc. Natl Acad. Sci. USA 2008 105(48): 18994-18999; 86) Panatier, A. et al. Cell 2011 146: 785-798; 87) Sloan, J. L. and Mager, S. J. Biol. Chem. 1999 274(34): 23740-23745; 88) Orru, M. et al. PloS One 2011 6(1): e16088; 89) Ferre, S. and Ciruela, F. J Caffeine Adenosine Res. 2019 9(3): 89-97; 90) Burbach, J. P. Eur. J. Pharmacol. 2010 626(1): 27-48; 91) Sipkova, J. et al. Physiol Res. 2017 66(5): 729-740; 92) Hokfelt, T. et al. Front Neural Circuits. 2018 12: 106; 93) Heuser, K. and Enger, R. Front Cell Neurosci 2021 15: 695380; 94) Verhoog, Q. P. et al. Front Neurol. 2020 11: 591690; 95) Farber, S. A. et al. Brain Res. 1996 723(1-2): 90-99; 96) Abbott, N. J. et al. Nat Rev Neurosci. 2006 7(1): 41-53; 97) Wong, C. G. T. et al. Toxicol Rev 2004 23(1): 3-20; 98) Ref. 234, Table 2; 99) Chi, Yu-M. et al. Biological and Pharmaceutical Bulletin. 2005 28(10): 1989-1991; 100) Wang, M.-L. et al. Sci Rep 2015 5: 16107; 101) Martinez-Pinilla, E. et al. 2015 Front Pharmacol. 6: 30; 102) Ref. 9, Table 17A; 103) Ref. 3, Table 18A; 104) Encyclopedia of Life Sciences Amino Acid Neurotransmitters. Jeremy M Henley, 2001 John Wiley & Sons, Ltd .; 105) Jurgensen, S. et al Pharmacology, Biochemistry and Behavior. 2005 81(3): 466-477; 106) Mohamed, A. F. et al. J. Pharm Pharmacol. 2000 52(12): 1553-1561; 107) Kang, T. H. et al. Life Sciences. 2004 76(3): 331-343; 108) Zhou, J. and Zhou, S. J Ethnopharmacol. 2010 132(1): 15-27; 109) Lin, J.-C. et al. Prog Neuropharmacol Biol Psychiatry 2016 71: 7-13; 110) Poleszak, E. Pharmacol Rep 2008 60(4): 483-489; 111) Ref. 238, Table 2; 112) Khanfar, M. A. et al. Front Neurosci 2016 10: 201; 113) Pyke, R. E. Sex Med Rev 2020 8(2): 183-190; 114) Ref. 241, Table 2; 115) Ref. 33, Table 14; 116) Kennedy, D. O. and Wightman, E. L. Adv Nutr 2011 2(1): 32-50; 117) Lu, K. et al. Hum Psychopharmacol 2004 19(7): 457-465; 118) Malykh, A. G. and Sadaie, M. R. Drugs 2010 70(3): 287-312; 119) Dingledine, R. et al., Pharmacol Rev 1999 51(1): 7-61; 120) Taylor, C. P. et al. Pharmacol Ther 2016 164: 170-182; 121) Schuehly, W. et al., Chem Biol. 2011 18(8): 1053-1064; 122) Huang, R. Q. et al. Brain Res Mol Brain Res. 67(1): 177-183, 1999; 123) Huang, R. et al. Neuropharmacology 58(8)1246-1251, 2010; 124) Ochoa-de la Paz, L. et al. Expert Rev Neurother 19(4): 289-291, 2019; 125) Alvarez, L. D. and Pecci, A. J. Steroid Biochem Mol Biol. 182: 72-80, 2018; 126) Bourier, G. et al. Curr Opin Neurobiol. 51: 1-7, 2018; 127) Rodriguez-Moreno, A. et al. Neuron 77(1): 35-42, 2013; 128) Ref. 235, Table 2

For mammals, a vesicular neurotransmitter transporter (“VNT”) moves a neurotransmitter into a vesicle for release from neurons into the extrasynaptic space, and a typically a neuron's vesicle that releases a neurotransmitter into a synapse (e.g., synapse cleft). A glial cell (e.g., astrocyte) also use VNT(s) to move a neurotransmitter (e.g., Glu, GABA, ATP, D-Ser) into a vesicle for release into an extrasynaptic region/synaptic cleft. As used herein both such neuron/glial cell neurotransmitter vesicle that releases a neurotransmitter in a synapse may be referred to as a “synaptic vesicle.” A vesicle that contains one or more neurotransmitter(s) are typically named for the neurotransmitter(s), such as a “GABAergic” vesicle that contains GABA, a “Serotonergic/Noradrenalinergic” vesicle that contains serotonin and noradrenaline, though often a vesicle may be referred to by a single prominent neurotransmitter of one or more that the vesicle contains. Examples of a VNT include VGAT for moving GABA, VEAT/VGLUT1/VGLUT2/VGLUT3 for moving Glu, VMAT2/VMAT2 for moving a monoamine, VAChT for moving acetylcholine, VPAT for moving a polyamine, and VNUT for moving a nucleotide into a vesicle. Movement of a neurotransmitter by a VNT into a vesicle is often promoted by the activity of another protein that moves an ion into/out of a vesicle, such as vescular chloride channel protein (e.g., CLC-3, CLC-4) and/or vesicular ATPase. For mammals, some vesicles contain two or more neurotransmitters (e.g., GABA and Glu; acetylcholine and Glu) that are released simultaneously (“corelease”) and/or some brain cells (e.g., presynaptic neuron, astrocyte) have vesicles each containing a single neurotransmitter (e.g., a vesicle with GABA, a vesicle with acetylcholine) wherein the vesicles are released together (“cotransmission”). For mammals, certain vesicle proteins (e.g., SV2A, SV2B, synaptotagmin-1) interact with plasma membrane SNARE proteins (e.g., synaptobrevin, syntaxin, SNAP-25) as well as complexin 1 (“synaphin-2”)/complexin 2 (“synaphin-1”), and possibly munc proteins (e.g., Munc13, Munc18 proteins) to allow fusion of a vesicle with the plasma membrane and release of a neurotransmitters into the extracellular space. For mammals, a neuron/glial cells may release neurotransmitters into the extracellular space by other mechanisms than a vesicle such as a transporter. For example, the transporter Xc- releases Glu, and other transporters such as GLYT1 may release Gly, ASC1 may release Ser/D-Ser, and BEST1 may release GABA. A glial cell's released neurotransmitter typically are in an extrasynaptic location to activate a neuron's presynaptic/postsynaptic receptor often to reduce/reverse a neuron's synaptic neurotransmitter signaling, though in some instances the glial cell's neurotransmitter may increase synaptic neurotransmission initiated signaling by activation of an extrasynpatic receptor [Ref 16, 148, 166, 212, 213, 214, 215, 216, 217, 218, 219, 220, 221, 222, and 223, Table 2; Ref 21, Table 7; Ref 11, Table 12; Ref 24, Table 5; Ref 35, Table 4]. For example, phasic inhibition of a neuron refers to a presynaptic neuron's release (e.g., release by a vesicle) of a neurotransmitter (e.g., GABA) to bind a post-synaptic inhibitory receptor (e.g., GABAA-R) to promote inhibitory neurotransmission signalling. Tonic inhibition of a neuron refers to a neurotransmitter (e.g., GABA) binding an extrasynaptic inhibitory receptor (e.g., GABAA-R) to promote persistent activation of an inhibitory receptor by the neurotransmitter. For example, some histaminergic neurons release histamine and GABA in cotransmission, and the released GABA may increase tonic inhibition by binding a neuron's extrasynaptic GABAA-R [Ref. 77, 212, and 213, Table 2; Ref 24, Table 5; Ref 35, Table 4].

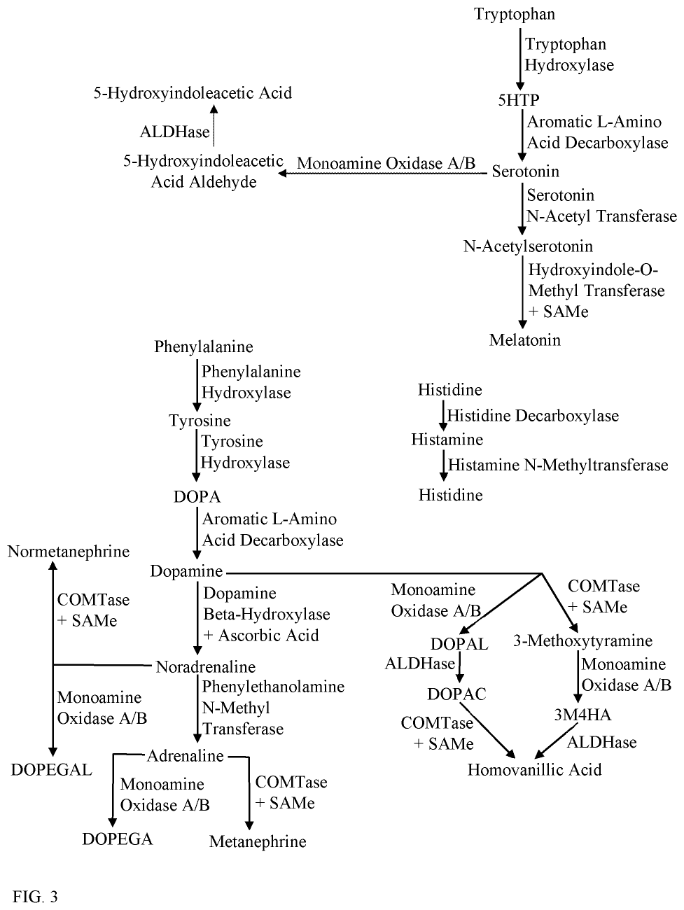

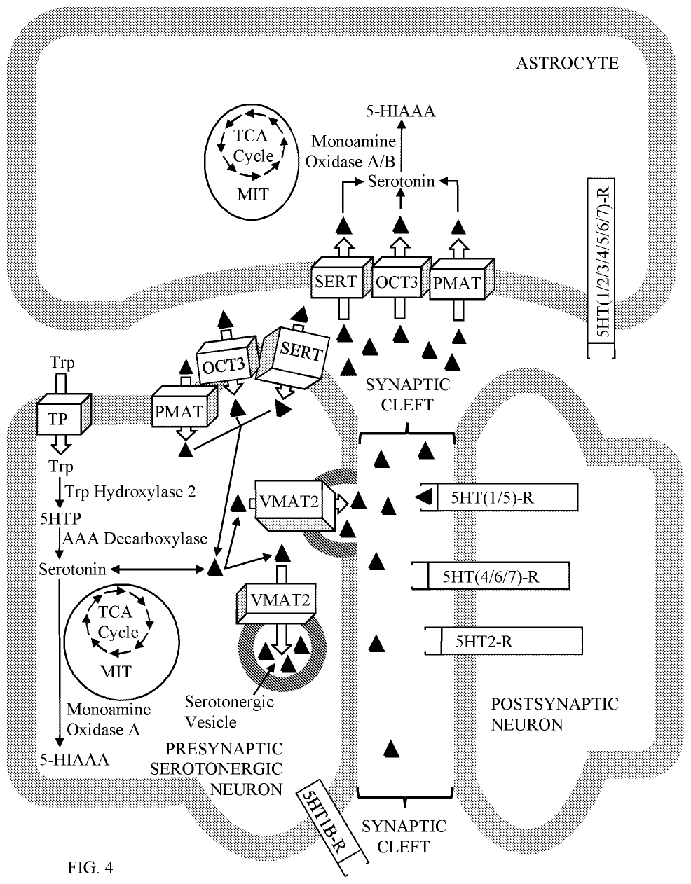

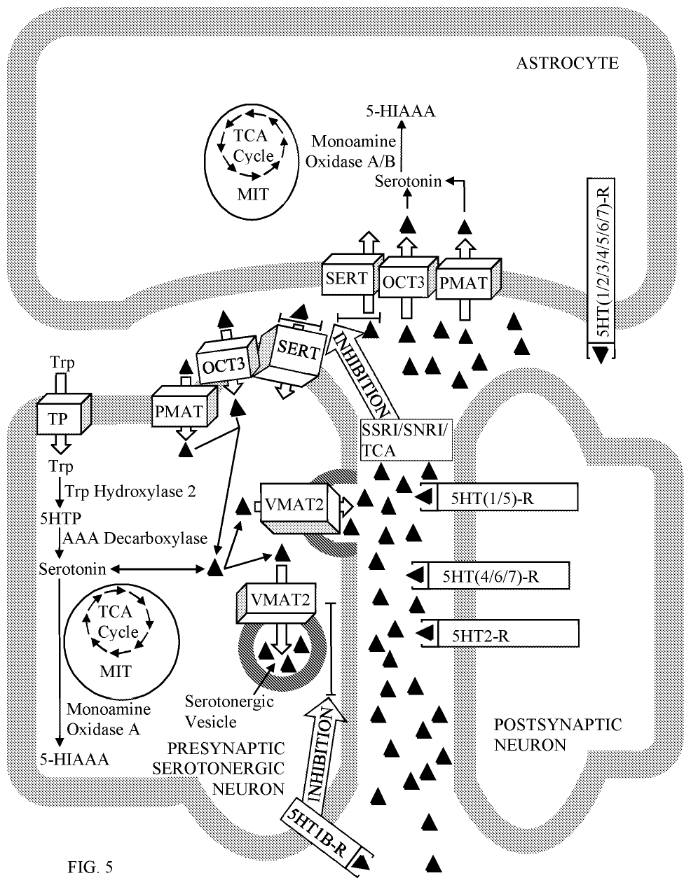

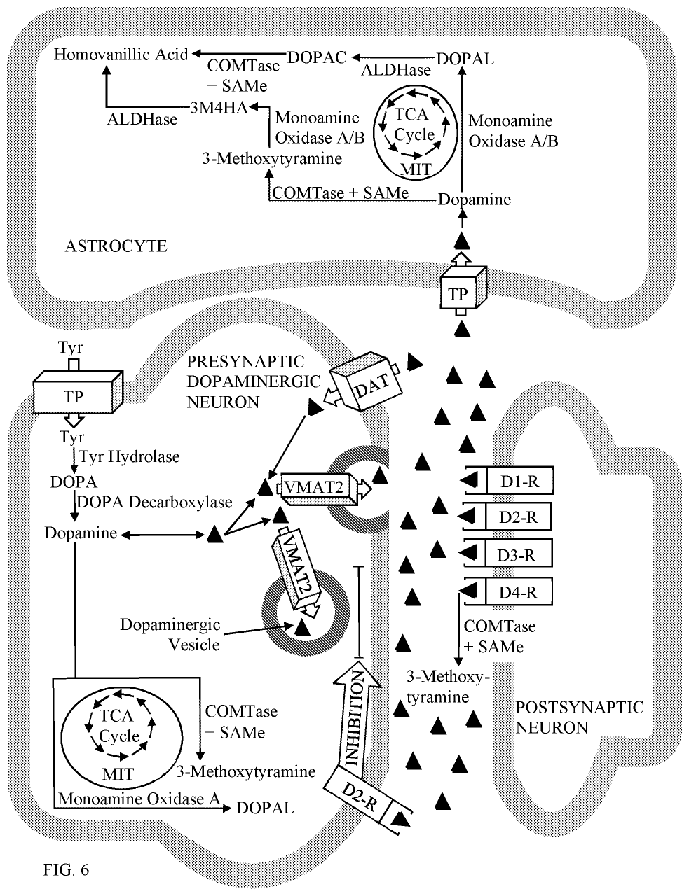

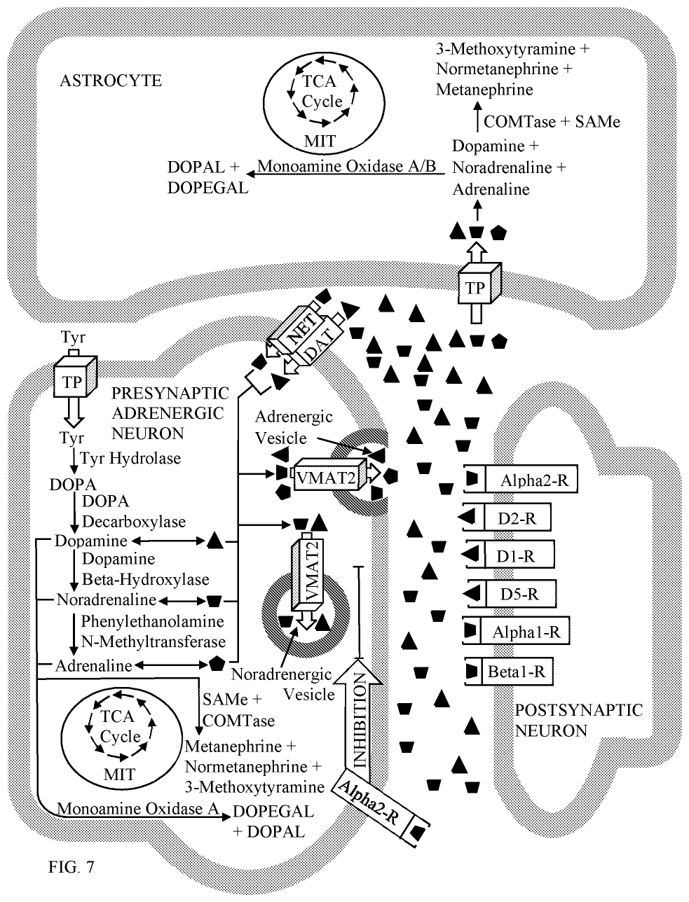

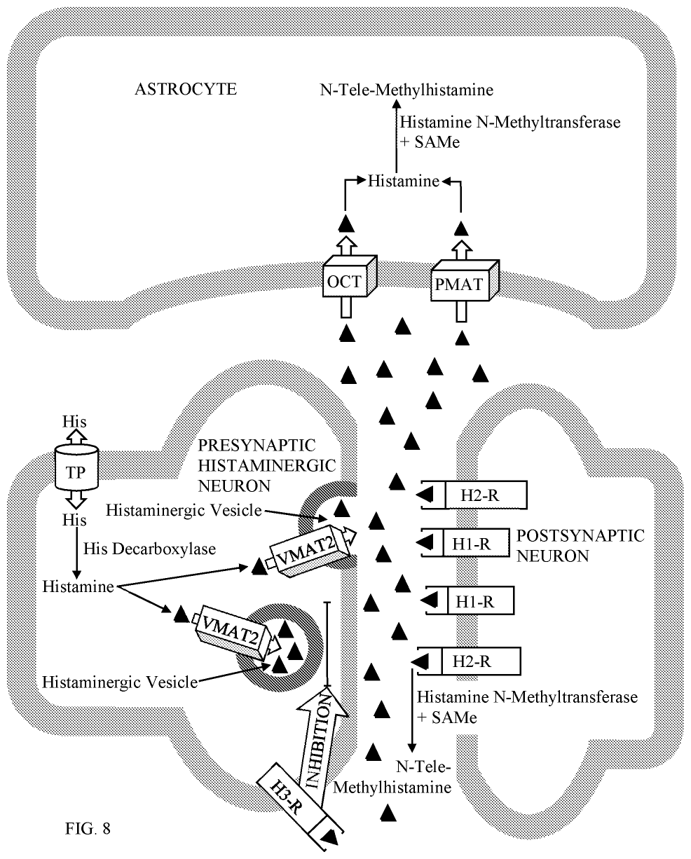

Tables 1, 2, 4, and 5 show transporters, receptors, and enzymes that are involved in monoamine creation and/or degradation reactions and neurotransmitter signaling, and the various ATS and ITS for these proteins, with many of these proteins depicted in FIG. 3, FIG. 4, FIG. 5, FIG. 6, FIG. 7 and FIG. 8. FIG. 3 depicts the metabolism (e.g., creation/degradation) of monoamines including serotonin, dopamine, histamine, noradrenaline, adrenaline, and melatonin. FIG. 4 depicts the transporter mediated movement and enzymatic conversion of chemicals (e.g., treatment substances) in neurons and astrocytes involved in serotoninergic neurotransmission. Trp, a treatment substance that is a precursor to new serotonin synthesis, is moved by a transporter (“TP”) into a presynaptic serotoninergic neuron and enzymatically converted into the neurotransmitter serotonin. Serotonin, depicted as a triangle, is moved by the VMAT2 vesicular transporter into serotoninergic vesicles, and fusion of a serotoninergic vesicle with the presynaptic neuron's plasma membrane facing the synaptic cleft releases serotonin into the synaptic cleft. Released synaptic serotonin binds the postsynaptic neuron's 5HT1-R/5HT5-R [“5HT(1/5)-R”] facing the synaptic cleft for synaptic serotoninergic signaling. For mammals, removal of a neurotransmitter from the extracellular space (e.g., near presynaptic neurons, near postsynaptic neurons, near the synaptic cleft) allows a neurotransmitter receptor to more effectively communicate a new signal by binding of the next release of a neurotransmitter into the extracellular space [Ref. 205, Table 2]. The serotonin may contact a transporter such as SERT/OCT3/PMAT, typically after diffusing out of the synaptic cleft, located on an astrocyte/neuron to be reuptaken into the astrocyte/neuron. The reuptaken serotonin is metabolized (i.e., enzymatically degraded by a monoamine oxidase) in an astrocyte into another chemical, 5-hydroxyindoleacetic acid aldehyde (“5-HIAAA”), though in a neuron (e.g., presynaptic serotoninergic neuron) the serotonin may be degraded into 5-HIAAA and/or be moved by VMAT2 into a serotoninergic vesicle for release again, though often a newly synthesized neurotransmitter is preferentially moved into a vesicle rather than a reuptaken neurotransmitter. As used herein, for example, Trp would function as a positive serotoninergic treatment substance by promoting serotonin creation in a presynaptic neuron, and the newly synthesized serotonin may be preferentially moved into vesicles for synaptic release and synaptic serotonergic signaling. As would be understood by one of ordinary skill in the art, this pattern described above of a neurotransmitter (e.g., serotonin) synthesis in a presynaptic neuron, movement into a synaptic vesicle, release into the synaptic cleft (which is not also labeled in other Figures for the sake of brevity), activation of a postsynaptic neuron's receptor, movement from the synaptic cleft into the extrasynaptic space, reuptake by synaptic facing/extrasynaptic transporter(s) into an astrocyte/neuron, and degradation in an astrocyte/neuron is common to most neurotransmitters described in the text herein and/or depicted in a Figure herein, and for the sake brevity this common pattern will not be described repeatedly herein and/or depicted herein for every neurotransmitter described herein, though aspects, particularly variations to this pattern as would be known to one of ordinally skill in the art, may also be described herein and/or may be depicted in a Figure. For example, variations to this pattern that would be known to those of ordinary skill in the art may be described herein and/or depicted herein include, for example, a neurotransmitter such as D-Ser being reuptaken in an astrocyte and then being released by a vesicle from the astrocyte into the synaptic cleft to activate a post-synaptic neuron's receptor; a neurotransmitter such as Glu being reuptaken by an astrocyte and being released by an Xc- transporter into the extrasynaptic space to activate a presynaptic/postsynaptic neuron's receptor; and/or a neurotransmitter such as GABA being synthesized in an astrocyte and then released into the extrasynaptic to activate a postsynaptic neuron's receptor, etc.

FIG. 5 is related to FIG. 4 and depicts the increase of extracellular serotonin when a selective serotonin reuptake inhibitor (“SSRI”) such as paroxetine (“Paxil”), a serotonin-noradrenaline reuptake inhibitor (“SNRI”) and/or a tricyclic antidepressant (“TCA”) inhibits SERT's reuptake of serotonin (depicted as a bar between SERT and serotonin) into the astrocytes/neurons. The increased amount of serotonin contacts and activates postsynaptic serotonin receptors [e.g., 5HT(1/5)-R; 5HT(4/6/7)-R, etc.] more frequently to further activate a postsynaptic neuron's serotoninergic signaling as well as move by diffusion from the synaptic cleft into the extrasynaptic space to activate a serotonin receptor [e.g., 5HT(1/2/3/4/5/6/7)-R] on an astrocyte. At increased levels of serotonin an extrasynaptic receptor (e.g., 5HT1B-R) on the presynaptic neuron becomes activated by serotonin to inhibit release of serotonin (depicted as a bar in front of the serotoninergic vesicle) from the presynaptic neuron to reduce the amount of postsynaptic serotoninergic responses. This pattern of increasing the synaptic/extrasynaptic amount of a neurotransmitter promoting the activation of a synpaptic/extrasynaptic receptor, particularly a receptor that inhibits release of additional neurotransmitter from a presynaptic neuron, is also common to various neurotransmitters as would be understood by one of ordinary skill in the art and for the sake of brevity may not be described and/or depicted in a Figure herein for every neurotransmitter described herein. A treatment substance, for example, that increases synaptic serotonin by reducing serotonin reuptake by SERT and/or inhibits monoamine oxidase degradation of serotonin to promote increased synaptic serotonin accumulation, and would be considered a positive serotoninergic treatment substance. However, excess accumulation of serotonin that activates a presynaptic neuron's extrasynpatic 5HR1B-R may reduce synaptic serotoninergic signaling so that the same treatment substance may function as a negative serotoninergic treatment substance as well (a “positive/negative serotoninergic” treatment substance). A treatment substance that activates the presynaptic neuron's extrasynpatic 5HT1B-R receptor (e.g., a 5HT1B-R Agonist) to inhibit synaptic release of serotonin would be considered herein to be negative serotoninergic treatment substance. And similarly for example, as used herein for each particular neurotransmitter, a treatment substance that is a metabolic precursor/prodrug for increasing a neurotransmitter's amount for synaptic neurotransmission is generally considered herein a positive neurotransmitterergic; a treatment substance that reduces degradation/reuptake for a synaptic neurotransmitter considered a positive neurotransmitterergic; a treatment substance that increases degradation/reuptake of a synaptic neurotransmitter considered a negative neurotransmitterergic, a treatment substance that activates a receptor that reduces synaptic release of neurotransmitter considered a negative neurotransmitterergic, a treatment substance that activates a receptor that reduces a neurotransmitter's synaptic signaling (e.g., a postsynaptic neuron's synaptic/extrasynaptic receptor whose activation reduces synaptic signaling) considered a negative neurotransmitterergic; a treatment substance that activates a receptor that increases a neurotransmitter's synaptic signaling (e.g., a postsynaptic neuron's synaptic/extrasynaptic receptor whose activation increases synaptic signaling) considered a positive neurotransmitterergic; and combinations thereof (e.g., a positive/negative neurotransmitterergic treatment substance).

FIG. 6 depicts the general location of various transporter proteins, enzymes, and receptors involved in dopamine production, degradation, release, reuptake, and/or receptor binding for neurons and astrocytes. Dopamine, depicted as a triangle, when reaching high extrasynaptic levels activates a presynaptic neuron's extrasynaptic D2-R receptor that inhibits (depicted as a bar in front of a synaptic vesicle) synaptic neurotransmitter release. A treatment substance, for example, that is a precursor to new dopamine synthesis (e.g., Try, DOPA) for synaptic release would be considered herein as a positive dopaminergic treatment substance; a treatment substance that is an inhibitor of synaptic dopamine degradation [e.g., a catechol-O-methyltransferase (“COMTase”) inhibitor, a monoamine oxidase inhibitor) and/or an inhibitor of DAT reuptake of synaptic dopamine would be considered a positive dopaminergic treatment substance as well as a possibly negative dopaminergic treatment substance by increased activation of the presynaptic neuron's extrasynaptic dopamine D2-R; a treatment substance that specifically activates the presynaptic neuron's extrasynaptic dopamine D2-R receptor would be considered herein to be a negative dopaminergic treatment substance; and a treatment substance that promotes degradation of synaptic dopamine (e.g., SAMe as a substrate with dopamine for COMTase) would be considered herein to have a negative dopaminergic activity. FIG. 7 depicts the general location of various transporter proteins, enzymes, and receptors involved in dopamine, noradrenaline, and adrenaline production, degradation, release, reuptake, and/or receptor binding for neurons and astrocytes. Activation of a presynaptic neuron's extrasynaptic Alpha2-R receptor inhibits (depicted as a bar in front of a synaptic vesicle) synaptic neurotransmitter release. FIG. 8 depicts the general location of various transporter proteins, enzymes, and receptors involved in histamine production, degradation, release, reuptake, and/or receptor binding for neurons and astrocytes. Activation of a presynaptic neuron's extrasynaptic H3-R receptor inhibits (depicted as a bar in front of a synaptic vesicle) synaptic neurotransmitter release.

TABLE 5
Enzymes Involved in Monoamine Metabolism
Enzyme: (EC no; “AAA”; Cofactor) [Location]

| | |
|---|---|
| Substrate for Enzyme Reaction | (“S”): Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Tryptophan Dioxygenase: (EC 1.13.11.11) [Liver, Reduces Blood Trp Amount]

| | |
|---|---|
| S: Trp | P: N-Formylkynurenine |

ATS: Trp; Tyr; Phe; His; Kynurenine [5]
Tyrosine Aminotransferase: (EC 2.6.1.5; Cofactor: Pyridoxal 5′ Phosphate) [Liver, Reduces
Blood Tyr Amount]

| | |
|---|---|
| S: Tyr + AKG | P: 4-Hydroxyphenylpyruvate + Glu |

Phenylalanine Hydroxylase: (EC 1.14.16.1; Cofactor: Tetrahydrobiopterin) [Liver, Reduces
Blood Phe Amount]

| | |
|---|---|
| S: Phe | P: Tyr |

ITS: Met; Norleucine; Trp; 3,4-Dihydroxystyrene [5]
Tyrosine Hydroxylase: (EC 1.14.16.2; Cofactor: Tetrahydrobiopterin)
[Dopaminergic/Noradrenergic Neuron]

| | |
|---|---|
| S: Tyr | P: 3,4-Dihydroxyphenylalanine (“DOPA”) |

ITS: Catecholamine; Phe; DOPA/Dopamine [Allosteric Inhibitor]; Panax ginseng Preparation
[Active: Saponin (Reduces Tyrosine Hydroxylase Amount)]; Rx{Metirosine} [5, 30]
ATS: Rosmarinus officinalis Preparation [Increases Tyrosine Hydroxylase Amount] [30]
Aromatic L-Amino Acid Decarboxylase: (EC 4.1.1.28; “AAA Decarboxylase”; Cofactor:
Pyridoxine) [Serotoninergic/Dopaminergic/Catecholaminergic Neuron]

| | |
|---|---|
| S: DOPA | P: Dopamine |
| S: 5HTP | P: Serotonin (“5HT”) |
| S: Phe | P: Phenethylamine (a neuromodulator) |
| S: Tyr | P: Tyramine (a neuromodulator) |
| S: Trp | P: Tryptamine (a neuromodulator) |

ITS: Genistein; Glycine max Preparation/Pueraria mirifica Preparation [Active: Genistein];
Rx{Benserazide, Carbidopa, Methyldopa }
Dopamine Beta-Hydroxylase: (EC 1.14.17.1)

| | |
|---|---|
| S: Dopamine + Ascorbic Acid | P: Noradrenaline + Dehydroascorbic Acid |

ITS: Panax ginseng Preparation [Active: Saponin (Reduces Dopamine-Beta-Hydroxylase
Amount)]; Rx{Disulfiram, Dopastin, Fusaric Acid, Nepicastat, Phenopicolinic Acid} [30]
Phenylethanolamine N-Methyl Transferase: (EC 2.1.1.28) [Adrenergic Neuron]

| | |
|---|---|
| S: Noradrenaline + S-adenosyl-methionine | P: Adrenaline + S-adenosyl-homocysteine |
| (“SAMe”) | (“SAH”) |

Tryptophan Hydroxylase 2: (EC 1.14.16.4; Cofactor: Tetrahydrobiopterin) [Neuron,
Serotoninergic Neuron, Monoaminergic Neuron]

| | |
|---|---|
| S: Trp | P: 5-Hydroxytryptophan (“5HTP”) |

ITS: DHEA; Rx{Telotristat ethyl} [2]
Serotonin N-Acetyl Transferase: (EC 2.3.1.87) [Pineal Gland, Pituitary Gland]

| | |
|---|---|
| S: Serotonin + Acetyl-CoA | P: N-Acetylserotonin + CoA |

Hydroxyindole-O-Methyl Transferase: (EC 2.1.1.4) [Pineal Gland]

| | |
|---|---|
| S: N-Acetylserotonin + SAMe | P: N-Acetyl-5-Methoxytryptamine |
| | (“Melatonin”) + SAH |

Monoamine Oxidase A/B: (EC 1.4.3.4; Cofactor: FAD) [Monoamine Oxidase A (“MAO A,”
in Neuron, Glial Cell, Astrocyte, Mitochondria)]; [Monoamine Oxidase B (“MAO B,” in Glial
Cell, Astrocyte, BBB Endothelial Cell, Mitochondria), See Monoamine Oxidase B at Table 8]

| | |
|---|---|
| S: Dopamine | P: 3,4-Dihydroxyphenylacetaldehyde |
| | (“DOPAL”) |
| S: Noradrenaline/Adrenaline | P: 3,4-Dihydroxyphenylglycolaldehyde |
| | (“DOPEGAL”) |
| S: Serotonin | P: 5-Hydroxyindoleacetic Acid Aldehyde |
| | (“5-HIAAA”) |
| S: 3-Methoxytyramine | P: 3-Methoxy-4-Hydroxphenyl-Acetaldehyde |

ITS: Monoamine Oxidase A/B: Scutellaria genus Preparation [Active: Baicalin]; Hypericum
perforatum Preparation; Agmatine; Rx{Phenelzine}; Monoamine Oxidase A: Harmine;
Harmaline; Harmane; Norharman; Peganum harmala Preparation [Active: Harmine, Harmaline,
Harmane, Norharman]; Melissa officinalis Preparation [Active: Citranellal, Geraniol]; Rhodiola
rosea Preparation [Active: Salidrosid, Tyrosol, Rosavin]; Rx{Ladostigil, Amiflamine,
Bazinaprine, Esuprone, Moclobemide} [29]
Catechol-O-Methyltransferase: (EC 2.1.1.6; “COMTase”)

| | |
|---|---|
| S: Dopamine + SAMe | P: 3-Methoxytyramine + SAH |
| S: Noradrenaline + SAMe | P: Normetanephrine + SAH |
| S: Adrenaline + SAMe | P: Metanephrine + SAH |
| S: 3,4-Dihydroxyphenylacetic Acid | P: Homovanillic Acid + SAH |
| (“DOPAC”) + SAMe | |

ITS: Zanthoxylum Clava-herculis Preparation [Active: Magnoflorine, Candicine, Lauriflorine,
Nitidine]; Rx{Entacapone, Tolcapone}
Aldehyde Dehydrogenase: (EC 1.2.1.3, “ALDHase”)

| | |
|---|---|
| S: 3,4-Dihydroxyphenylacetaldehyde | P: 3,4-Dihydroxyphenylacetic Acid |
| (“DOPAL”) | (“DOPAC”) |
| S: 3-Methoxy-4-Hydroxphenyl-Acetaldehyde | P: Homovanillic Acid |
| (“3M4HA”) | |
| S: 5-Hydroxyindoleacetic Acid Aldehyde | P: 5-Hydroxyindoleacetic Acid |
| (“5-HIAAA”) | |

ITS: Gastrodia elata Preparation [Active: Gastrol, Gastrodin, Bis(4-hydroxybenzyl)sulfide,
N6-(4-hydroxybenzyl)adenine Riboside (Possible Inhibitor)]
Histidine Decarboxylase: (EC 4.1.1.22; Cofactor: Pyridoxal 5′ Phosphate) [Histaminergic
Neuron]

| | |
|---|---|
| S: His | P: Histamine |

ITS: Catechin; Naringenin; Citrus junos Preparation/Citrus paradisi/sinensis Preparation
[Active: Naringenin]; Rx{Meciadanol, Tritoqualine}
Histamine N-Methyltransferase: (EC 2.1.1.8) [Astrocyte; Extracellular Space; Neuron]

| | |
|---|---|
| S: Histamine + SAMe | P: N-Tele-MethylHistamine + SAH |

ITS: Harmaline; Peganum harmala Preparation [Active: Harmaline]; Diphenhydramine;
Rx{Quinacrine}
1) Stanton, C. H. et al. Trends Neurosci. 2019 42(1): 23-42; 2) Ref. 1, Table 4; 3) Blakely, R. D. and Edwards, R. H. Cold Spring Harb Perspect Biol 2012 4(2): a005595; 4) Sitte, H. H. and Freissmuth, M. J Neurochem 2010 112(2): 340-355; 5) Cansev, M. & Wurtman, J. (2007). Aromatic amino acids in the brain. Handbook of Neurochemistry and Molecular Neurobiology. 6. 60-97; 6) Cho, H.-U. et al. Exp Mol Med. 2021 Jul 9; 7) Boison, D. Pharmacol. Rev. 2013 65(3): 906-943; 8) Wang, F.-Y.et al. J Pharm Anal. 2021 11(1): 15-27; 9) Kitahama, K. et al. Neurosci Lett. 1985 53(3): 303-308; 10) Ref. 39, Table 4; 11) Tyson, T. D. P. et al. Transl Psychiatry 2017 7(1): e1003; 12) Chakraborty, C. et al. Appl Biochem Biotechnol 2012 167(4): 845-860; 13) Meiser, J. et al. Cell Commun Signal 2013 11: 34; 14) Matsumoto, M. et al. Neuroscience 2003 116: 127-137; 15) Eisenhofer, G. et al. Pharmacol Rev 2004 56(3): 331-349; 16) Cagle, B. S. et al. Curr Opin Toxicol 2019 13: 16-21; 17) Ref. 161, Table 2; 18) Slopien, R. et al. ScientificWorldJournal 2012 194845; 19) Millan, M. J. et al. Trends Pharmacol Sci 2008 29(9): 454-464; 20) Ref. 37, Table 4; 21) Berumen, L. C. Et al. ScientificWorldJournal 2012 2012: 823493; 22) Ref. 38, Table 4; 23) Gainetdinov, R. R. et al. Pharmacol Rev 2018 70(3): 549-620; 24) Scammell, T. E. et al. Sleep. 2019 42(1): zsy 183; 25) Ref. 102, Table 2; 26) Ref. 109, Table 2; 27) Hoffman, G. E. and Koban, M. PLOS One 2016 11(12): e0152252; 28) Holecek, M. Physiol Res. 2020 69(4): 555-564; 29) Ref. 9, Table 17A; 30) Ref. 241, Table 2

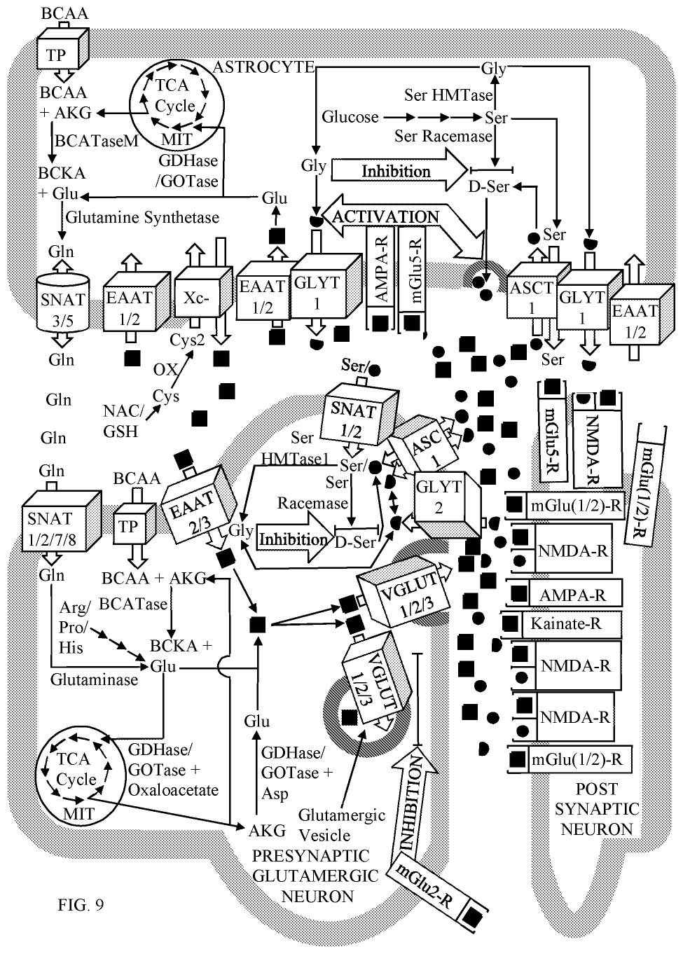

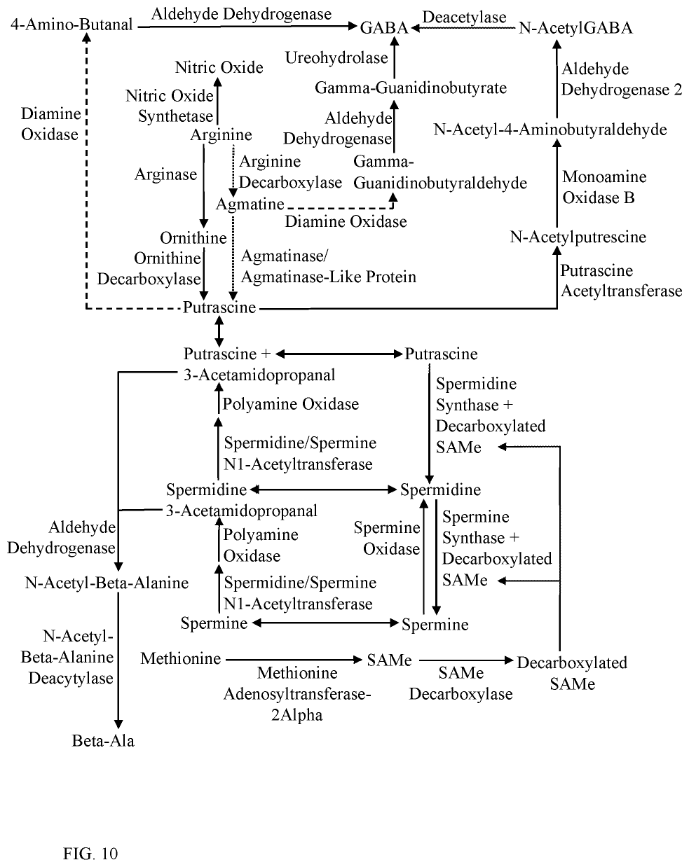

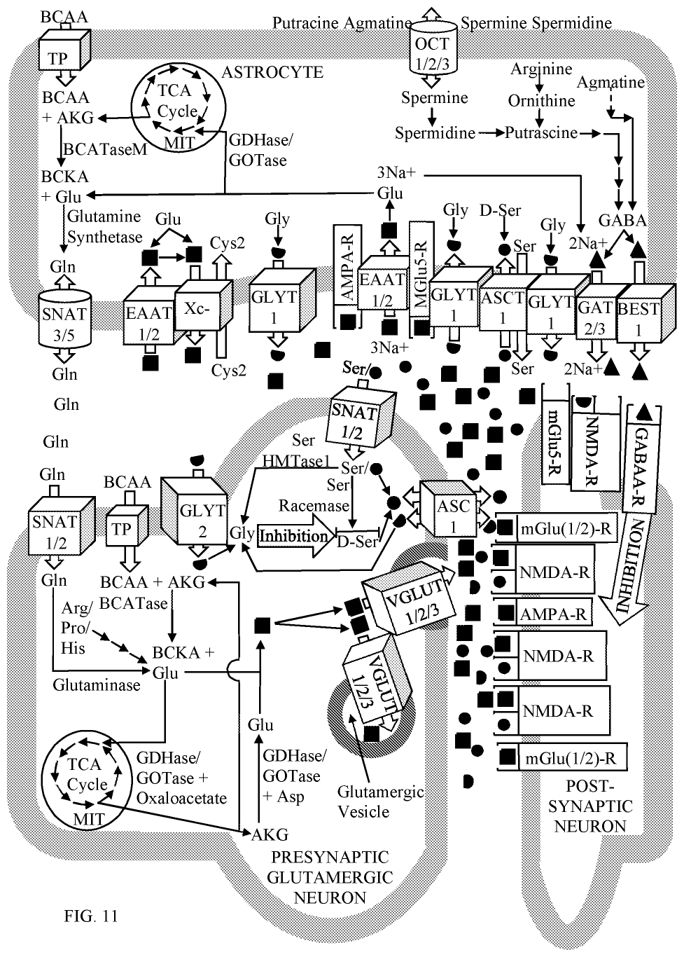

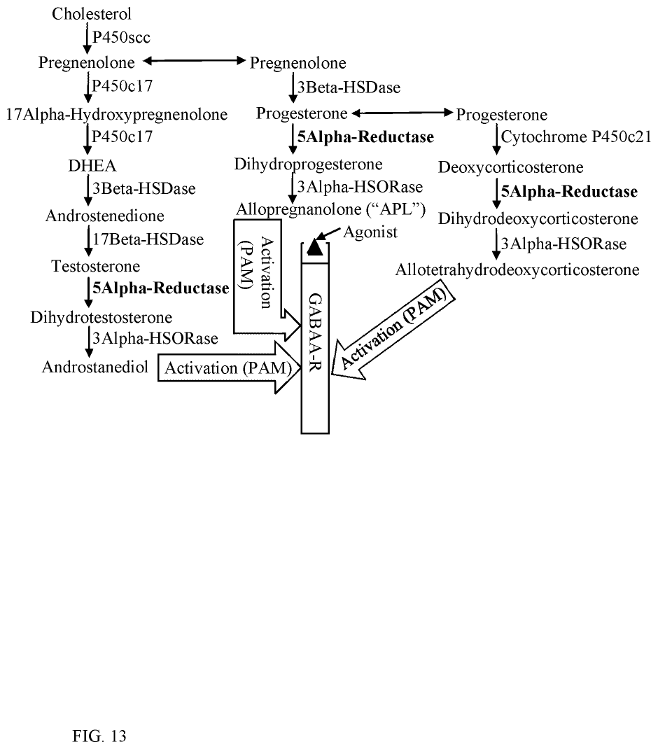

Tables 1, 2, 3, 4, 6, 7, 8, 9A, and 9B show transporters, receptors, and enzymes involved in Glu, GABA, Gln, Ser, D-Ser, neurosteroid creation/degradation, and polyamine creation/degradation; and the various ATS and ITS for these proteins, as these transporters, receptors, enzymes are involved in glutamatergic and/or GABAergic neurotransmitter signaling, with many of these proteins depicted in FIG. 9, FIG. 10, FIG. 11, FIG. 12, FIG. 13, FIG. 14, and FIG. 15. FIG. 9 depicts the transporter mediated movement and enzymatic conversion of chemicals (e.g., treatment substances) in neurons and astrocytes involved in glutamatergic responses. Depicted are the enzyme and transporter pathways for the synthesis and release of Glu, D-Ser, and Gly neurotransmitters into the extracellular space and the binding of these neurotransmitters to receptors to activate glutamatergic signaling. At increased levels of Glu, an extrasynaptic receptor (“mGlu2-R”) on the presynaptic neuron becomes activated by Glu to inhibit release of Glu from the presynaptic neuron (depicted as a bar in front of a glutamergic synaptic vesicle) to reduce the amount of postsynaptic glutamatergic responses. For humans, Glu is enzymatically degraded in the brain for energy production. For mammals, Glu and Gln are not readily transported across the BBB into the brain relative to other amino acids; with, for example, Phe and Leu being about 50%, and Gln about 5%, of the neutral amino acids that cross the BBB into the brain, with Leu being the fastest to cross the BBB. Neutral amino acids (e.g., BCAAs, Lys, Phe) that crosses the BBB from the blood and are moved into brain cells where enzymes catalyze reactions on BCAAs to transfer about 30% of the BCAAs' nitrogen into newly synthesized Glu to replace the enzymatically degraded Glu. For humans, SNAT3/SNAT5 transporter(s) moves Gln, which generally does not act as a neurotransmitter, out of glial cells (e.g., astrocyte, Bergmann glial cell) into the extracellular space. For mammals, SNAT1/SNAT2/SNAT7/SNAT8 transporter(s) moves Gln into neurons (e.g., presynaptic glutamatergic neuron, presynaptic GABAergic neuron). The presynaptic neurons enzymatically converted Gln to Glu, and for some neurons (e.g., GABAergic neurons) Glu is then enzymatically converted into GABA. Arg, Pro, and His are also metabolized into Glu in the brain [Ref 1, Table 3; Ref 4, 10, 14, 28, 34, 49, 50, 51, and 52, Table 6; Ref 1, 68, and 205, Table 2]. For mammals, NMDA-Rs are the main Glu receptors, and use coagonists (e.g., Gly, D-Ser) for activation. D-Ser is often a synaptic coagonist for an NMDA-R (e.g., synaptic NMDA-R having the GluN2A subunit) released from astrocytes and neurons (e.g., GABAergic neurons, glutamatergic neurons). Some synapse's NMDA-R use Gly as a coagonist, though Gly is often a coagonist for an extrasynaptic NMDA-R. Ser is made from glucose in glia cells (e.g., astrocytes), and Gly in brain is produced by Ser degradation. Ser in neurons is used to make D-Ser. Gly inhibits serine racemase production of D-Ser (depicted as a bar in FIG. 9). Glial and neuron cells supply D-Ser in the synapse and glial cell GLYT1 removes synaptic Gly; and glial cells may supply extrasynaptic Gly (e.g., by extrasynaptic GLYT1) for tonic activation of an extrasynaptic NMDA-R. ASC1 uptakes D-Ser into neurons, and ASC1 can also release D-Ser and Gly to activate synaptic NMDA-R. The activation of AMPA-R often depolarizes a cell (e.g., a neuron) to promote the ease of activation of an NMDA-R; and activation of an astrocyte AMPA-R may increase intracellular Na+ and promote GLYT1 release of Gly into the extracellular space. D-Ser is released by astrocytes by Glu receptor(s) (e.g., AMPA-R, Kainate-R, metabotropic Glu receptor) activation. GlyT2 moves Gly into inhibitory neurons' presynaptic terminals and axons from the extracellular space (e.g., synapses) [Ref. 86, 107, 119, 121, 122, and 160, Table 2; Ref. 49 and 50, Table 4; Ref. 2, 3, 4, 13, 14, 15, 16, 17, 18, 19, 20, and 21, Table 7]. For mammals, Glu metabotropic receptors (“mGlu-R”) produce varied actions. For example, Group I receptors (e.g., mGlu1-R, mGlu5-R) upon activation in astrocytes promotes gliotransmitter release, and activation inhibits K+ channels increasing Ca2+ inside the cell, and activates adenylyl cyclase/PKC. Activating a Group II Glu metabotropic receptor (e.g., mGlu2-R, mGlu3-R) activates K+ channel(s), inhibits adenylyl cyclase, and inhibits voltage-gated Ca2+ channel(s); and activating a Group III receptor (e.g., mGlu-R4, mGlu-R8, mGlu-R7) generally hyperpolarize a neuron by activating a K+ channel (e.g., GTRK channel), inhibits voltage gated Ca2+ channel(s), and inhibits adenylyl cyclase, though activating a mGlu-R3 may depolarize a neuron. Group II/III are typically located in the synapse of a presynaptic neuron's (though some on postsynaptic neurons) [Ref 15 and 86, Table 4]. For humans, EAAT transporters move (e.g., reuptake) Glu, Asp and D-Asp into cells. Astrocytes import about 80-90% of extracellular Glu by EAAT2. Much of the remaining Glu is taken into cells by the Glu receptors (post-synaptic neuron Glu receptors) and/or other EAAT transporter(s) [Ref. 11, 14, 48, 201, 202, 203, 204, 205, 206, 207, 208, and 209, Table 2].

TABLE 6
Enzyme Reactions for Glutamate Creation, Glutamate Degradation,
Glutamine Creation, GABA Creation, and GABA Degradation
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
AKG Dehydrogenase Complex: (EC 1.2.4.2; Cofactor: Alpha-Lipoic Acid, Thiamine
Pyrophosphate) [Mitochondria, TCA Cycle Enzyme]

| | |
|---|---|
| S: AKG + NAD+ + CoA | P: SuccinylCoA + NADH |

ITS: Asp [20]
ATS: Calcium/Ca2+ [Inside the Cell] [20]
SuccinylCoA Synthase: (EC 6.2.1.4) [Mitochondria, TCA Cycle Enzyme]

| | |
|---|---|
| S: SuccinylCoA + GDP | P: Succinate + GTP + CoA |

Succinic Dehydrogenase: (EC 1.3.5.1) [Mitochondria, TCA Cycle Enzyme]

| | |
|---|---|
| S: Succinate + FAD+ | P: Fumarate + FADH2 |

Fumarase: (EC 4.2.1.2) [Mitochondria, TCA Cycle Enzyme]

| | |
|---|---|
| S: Fumarate | P: Malate |

Malate Dehydrogenase: (EC 1.1.1.37) [Mitochondria, TCA Cycle Enzyme]

| | |
|---|---|
| S: Malate + NAD+ | P: Oxaloacetate + NADH |

Citrate Synthase: (EC 2.3.3.1) [Mitochondria, TCA Cycle Enzyme]

| | |
|---|---|
| S: Acetyl-CoA + Oxaloacetate | P: Citrate + CoA |

Aconitase: (EC 4.2.1.3) [Mitochondria, TCA Cycle Enzyme]

| | |
|---|---|
| S: Citrate | P: Isocitrate |

Isocitrate Dehydrogenase: (EC 1.1.1.42) [Mitochondria, TCA Cycle Enzyme]

| | |
|---|---|
| S: Isocitrate + NAD+ | P: AKG + NADH |

ATS: Calcium/Ca2+ [Inside the Cell] [20]
Branched-Chain Amino Acid Aminotransferase: (EC 2.6.1.42; “BCATase”; Cofactor:
Pyridoxal 5′ Phosphate) [For Human, Mitochondrial BCATase (“BCATaseM”) in BBB
Endothelial Cell; For Rodent, BCATaseM in Astrocyte; and Cytosolic BCATase
(“BCATaseC”) in Neuron Where the Reverse Reaction May Occur]

| | |
|---|---|
| S: AKG + Val | P: Glu + Alpha-Ketoisovalerate (“KIV”) |
| S: AKG + Ile | P: Glu + Alpha-Keto-Beta-Methylvalerate |
| | (“KMV”) |
| S: AKG + Leu | P: Glu + Alpha-Ketoisocaproate (“KIC”) |

ITS: Beta-Hydroxy Beta-Methylbutyrate [Reduces Leu Degradation into KIC by BCATase in
Muscle] [59]
Glutamine Synthetase: (EC 6.3.1.2; Cofactor: Manganese) [Mitochondria; Astrocyte;
Ependymal Cell; Oligodendrocyte; Interneuron, Neuron having Nitric Oxide Synthase]

| | |
|---|---|
| S: Glu | P: Gln |

Glutaminase: (EC 3.5.1.2) [Neuron, BBB Endothelial Cell]

| | |
|---|---|
| S: Gln | P: Glu |

ITS: Glu [Feedback Inhibitor]
ATS: Adenosine Dinucleotide; Trinucleotides (e.g., ATP), Succinate, Citrate
Glutamate Decarboxylase: (EC 4.1.1.15, “Glutamic Acid Decarboxylase”; Cofactor:
Pyridoxal 5′ Phosphate) [GABAergic Neuron]

| | |
|---|---|
| S: Glu | P: GABA |

ITS: Humulus lupulus Preparation [Active: Cohumulone, N-(+)-Adhumulone, Colupulone];
Apigenin; Matricaria camomilla Preparation [Active: Apigenin, Alpha-Bisabolol,
Apigenin-7-glucoside, Luteolin, Naringenin]; Rx{Aminooxyacetic Acid, Allylglycine} [22, 55]
ATS: Centella asiatica Preparation [Active: Asiaticoside, Asiatic acid]; Valeriana officinalis
Preparation [Active: Valerenic acid] [22]
Glutamate Dehydrogenase1: (EC 1.4.1.2; “GDHasel”) [Glial Cell, Astrocyte]

| | |
|---|---|
| S: Glu + NAD(P)+ | P: AKG + NAD(P)H |

ITS: Palmitoyl-CoA; Zinc/Zn2+; Epigallocatechin Gallate; NAM: Pregnenolone;
Progesterone; Corticosterone; DHEA; Dihydrotestosterone; Estradiol; Estriol [11];
ATS: PAM: Leu; Ile; Val
Glutamate Dehydrogenase2: (EC 1.4.1.2; “GDHase2”) [Astrocyte; Neuron, Presynaptic
Nerve Terminal; Mitochondria]

| | |
|---|---|
| S: Glu + NAD(P)+ | P: AKG + NAD(P)H |

ITS: NAM: Pregnenolone; Progesterone; Corticosterone; DHEA; Dihydrotestosterone;
Estradiol; Estriol [11]
Alanine Aminotransferase: (EC 2.6.1.2; “GPTase,” Amino Transaminase,”
“Glutamate-Pyruvate Transaminase”; Cofactor: Pyridoxal 5′ Phosphate) [Blood; Astrocyte;
Neuron, Reverse Reaction May Dominate in Neuron]

| | |
|---|---|
| S: AKG + Ala | P: Glu + Pyruvate |

ITS: Zanthoxylum Clava-herculis Preparation [Active: Chelerythrine]
Ornithine Aminotransferase: (EC 2.6.1.13) [Mitochondria]

| | |
|---|---|
| S: Orn + AKG | P: Glutamyl 5-Semialdehyde + Glu |

5-Oxoprolinase: (EC 3.5.2.9; “Pyroglutamase”)

| | |
|---|---|
| S: Pyroglutamate | P: Glu |

GABA Transaminase: (EC 2.6.1.19; “GABATase,” “GABA Aminotransferase”; Cofactor:
Pyridoxal 5′ Phosphate) [Mitochondria]

| | |
|---|---|
| S: GABA + AKG | P: Glu + Succinic Semialdehyde |

ITS: Rosmarinic Acid; Melissa officinalis Preparation [Preparation; Active: Citranellal,
Geraniol, Oleanolic Acid, Rosmarinic Acid, Ursolic Acid, Pentacyclic Ursolic Acid, (GABA
Transaminase Inhibitor; Rosmarinic Acid Reduces GABA Transaminase Amount)]; Centella
asiatica Preparation [Active: Asiaticoside]; Gastrodin; Gastrodia elata Preparation [Gastrodin
(Reduces GABA Transaminase Amount)]; Rx{Aminooxyacetic Acid, Vigabatrin, Cycloserine,
Isoniazid, Phenelzine, Valproate, Valproate Pivoxil, Valpromide} [21, 55, 56, 57, 58]
Succinic Semialdehyde Dehydrogenase: (EC 1.2.1.24)

| | |
|---|---|
| S: Succinic Semialdehyde + NAD+ | P: Succinate + NADH |

ITS: Gastrodin; Gastrodia elata Preparation [Active: Gastrodin (Reduces Succinic
Semialdehyde Dehydrogenase Amount)] [56]
Branched-Chain Alpha-Keto Acid Dehydrogenase Complex: (EC 1.2.4.4, Alpha-Ketoacid
Dehydrogenase; EC 2.3.1.168, Dihydrolipoamide Dehydrogenase; EC 1.8.1.4,
Dihydrolipoamide Dehydrogenase; Cofactor: Alpha-Lipoic Acid, Thiamine Pyrophosphate,
FAD, NAD+) [Mitochondria, Irreversible Alpha-Keto Acid Degradation for ATP Production]

| | |
|---|---|
| S: Alpha-Ketoisovalerate + Coenzyme A | P: Isobutyryl-CoA |
| S: Alpha-Keto-Beta-Methylvalerate + | P: 2-Methylbutanoyl-CoA |
| Coenzyme A | |
| S: Alpha-Ketoisocaproate + Coenzyme A | P: 3-Methylbutanoyl-CoA |

ATS: Caprylic Acid/Branched-Chain Keto-Acid [Reduces BCAA Degradation Through BCKA
Degradation by Branched Chain Alpha-Keto Acid Dehydrogenase Complex by Inhibiting
Branched Chain Alpha-Keto Acid Dehydrogenase Kinase that is a Branched-Chain Alpha-Keto
Acid Dehydrogenase Complex Inhibitor] [60, 61]
Glutamate Oxaloacetate Transaminase: (EC 2.6.1.1; “Aspartate Transaminase,” “GOTase”;
Cofactor: Pyridoxal 5′ Phosphate) [Neuron; Astrocyte; Blood; Mitochondria]

| | |
|---|---|
| S: Glu + Oxaloacetate | P: AKG + Asp |

ITS: Zanthoxylum Clava-herculis Preparation [Active: Chelerythrine]
ATS: Oxaloacetate [In Blood Reduces Blood Glu Levels Allowing More Movement of Glu
Across the BBB from the Brain into the Blood] [17]
ATP Citrate Lyase: (EC 4.1.3.8)

| | |
|---|---|
| S: Citrate + CoA | P: Acetyl-CoA + Oxaloacetate |

ITS: Hydroxycitric Acid; Garcinia cambogia Preparation [Active: Hydroxycitric Acid] [48]
1) Ref. 16, Table 2;
2) Nie, C. et al. Int J Mol Sci. 2018 19(4): 954;
3) Brosnan, J. T. and Brosnan, M. E. J Nutr 2006 136(1 Suppl): 207S-211S;
4) Cole, J. T. (2015) Metabolism of BCAAs. In: Rajendram R., Preedy V., Patel V. (Eds) Branched Chain Amino Acids in Clinical Nutrition. Nutrition and Health. Humana Press, New York, NY;
5) Harris, R. A. et al. Biochem Biophys Res Commun. 2004 313: 391-396;
6) Hull, J. et al. Neurochem Int. 2018 112: 49-58;
7) Calvetti, D. and Somersalo, E. Front. Endocrinol. (Lausanne) 2013 4: 137;
8) Ref. 1, Table 3;
9) Judge, A. and Dodd, M. S. Essays Biochem. 2020 64(4): 607-647;
10) Bak, L. K. et al J Neurochem. 2006 98(3): 641-653;
11) Spanaki, C. et al. Mol Cell Endocrinol. 2015 415: 1-11;
12) Hull, J. et al. J. Neurochem. 2012 123(6): 997-1009;
13) Bernstein, H.-G. et al. J. Chem Neuroanat. 2014 61-62: 33-50;
14) McKenna, M. C. Front Endocrinol (Lausanne) 2013 4: 191;
15) Cynober, L. Ann Nutr Metab 2018 73 Suppl 5: 5-14;
16) Rink, C. et al. Antioxid Redox Signal 2011 14(10): 1777-1785;
17) Ref. 175, Table 2;
18) Ref. 113, Table 2;
19) Ref. 114, Table 2;
20) Tretter, L. and Adam-Vizi, V. Philos Trans R Soc Lond B Biol Sci. 2005 360(1464): 2335-2345;
21) Awad, R. et al. Phytother Res 2009 23: 1075-1081;
22) Awad, R. et al. Can J Physiol Pharmacol. 2007 85(9): 933-942;
23) Mahan, V. L. Med Gas Res. 2019 9(1): 24-45;
24) Rivera, C. et al. J Physiol 2005 562(Pt 1): 27-36;
25) Provensi, G. et al. Int J Mol Sci 2019 20(19): 4724;
26) Kaila, K. et al. Curr Opin Neurobiol 2014 26: 34-41;
27) Hertz, L. and Chen, Y. Front Integr Neurosci. 2017 11: 18;
28) Hull, J. et al. J Neurosci Res. 2015 93(7): 987-998;
29) Ref. 167, Table 2;
30) Bak, L. K. et al. J Neurosci Res. 2007 85(15): 3465-3470;
31) Ref. 168, Table 2;
32) Cole, J. T. et al. 2010 Proc Natl Acad Sci U S A 107(1): 366-371;
33) Ref. 205, Table 2;
34) Yudkoff, M. Neurochem Res. 2017 42(1): 10-18;
35) Barresi, E. et al. J Enzyme Inhib Med Chem 2021 36(1): 1783-1797;
36) Karioti, A. et al. Molecules 2016 21(12): 1649;
37) Carta, F. et al. J Med. Chem. 2010 53: 5511-5522;
38) Innocenti, A. et al. Bioorg. Med. Chem. 2010 18: 2159-2164;
39) Ekinci, D. et al J. Enzyme Inhib. Med. Chem. 2013 28: 283-288;
40) Gidaro, M. C. et al. S. Planta Med. 2015 6: 533-540;
41) Mancuso, F. et al. ACS Med Chem Lett 2020 11(5): 1000-1005;
42) Provensi, G. et al. Int J Mol Sci 2019 20(19): 4724;
43) Temperini, C. et al. J. Med. Chem 49(10): 3019-3027;
44) Scozzafava, A. et al. Molecules 2016 21(12): 1726;
45) Supuran, C. T. Mol Divers 2011 15(2): 305-316;
46) Ruusuvuori, E. et al. EMBO J 2013 32(16): 2275-2286;
47) Zhao, H. et al. Prog Neurobiol 2016 138-140: 19-35;
48) Jena, B. S. et al. J Agric Food Chem 2002 50(1): 10-22;
49) Grill, V. et al. Metabolism. 1992 41(1): 28-32;
50) Hagglund, M. G. A. et al. J. Mol. Biol. 2015 427(6 Pt B): 1495-1512;
51) Cole, J. T. et al. 2010 Proc Natl Acad Sci USA 107(1): 366-371;
52) Mackenzie, B. and Erickson, J. D. Pflugers Arch. 2004 447 (5): 784-795;
53) Spanaki, C. et al. Mol Neurobiol. 2016 53(8): 5180-5188;
54) Erlander, M. G. et al. Neuron. 1991 7(1): 91-100;
55) Ref. 9, Table 17A;
56) An, S. J. et al. J Neurosci Res 2003 71(4): 534-543;
57) Ref. 33, Table 14;
58) Ref. 116, Table 4;
59) Holecek, M. J. Cachexia Sarcopenia Muscle 2017 8(4): 529-541;
60) Ref. 4, Table 6; 61) Ref. 1, Table 3

TABLE 7
For Mammals, Enzymes Involved in Serine and D-Serine Metabolism
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Serine Racemase: (EC 5.1.1.18; Cofactor: Pyridoxal 5′ Phosphate)
[Glutamatergic/GABAergic/Purkinje Neuron; Astrocyte]

| | |
|---|---|
| S: Ser | P: D-Ser |
| S: D-Ser/Ser | P: Pyruvate |

ITS: Malonate; Gly; Asn; Asp; Cys; Nitric Oxide [Promotes Nitrosylation of Serine
Racemase], Madecassoside; Madecassic Acid; Oxaloacetic Acid; Hydroxyproline [1-6]
ATS: ATP; Magnesium/Mg2+; Calcium/Ca2+; ADP; GTP; GSH [1, 2, 3, 4]
D-Amino Acid Oxidase: (EC 1.4.3.3; Cofactor: FAD) [Glia Cell, Bergmann Cell Adjacent to
Purkinje Neuron; Neuron]

| | |
|---|---|
| S: D-Ser | P: Imino Pyruvic Acid (Pyruvate precursor) |

ATS: Nitric Oxide [7, 8]
Neuronal Nitric Oxide Synthase: (EC 1.14.13.39; Cofactor: FAD, FMN, Heme,
Tetrahydrobiopterin, Calmodulin) [Neuron, Postsynaptic Neuron]

| | |
|---|---|
| S: 2 Arg + 3 NADPH | P: 2 Nitric Oxide + 2 Citrulline + 3 NADPH |

ITS: Agmatine; Panax genus Preparation (Active: Ginsenoside Rb1/Rg1); Aminoguanidine
[10, 11, 12]
ATS: Increased Ca2+ [4]
Ser/Gly Hydroxymethyltransferase: (EC 2.1.2.1; “Ser HMTase” e.g., Ser HMTase 1)
[Astrocyte]

| | |
|---|---|
| S: Gly + 5,10-Methylenetetrahydrofolate | P: Ser + Tetrahydrofolate |

1) Raboni, S. et al. Front Mol Biosci. 2019 5: 112;
2) Jiraskova-Vanickova, J. et al. Curr Drug Targets. 2011 12(7): 1037-1055;
3) Toney, M. D. Biochim Biophys Acta. 2011 1814(11): 1407-1418;
4) Pollegioni, L. and Sacchi, S. Cell Mol Life Sci 2010 67(14): 2387-2404;
5) Dunlop, D. S. and Neidle, A. Brain Res Mol Brain Res. 2005 133(2): 208-214;
6) Rani, K. et al. Sci Rep. 2020 10: 4640;
7) Horiike, K. et al. Brain Res Bull. 1987 19(5): 587-596;
8) Wu, Q. et al. Aging Dis 2020 11(6): 1640-1653;
9) Ref. 122, Table 2;
10) Seiler, N. Amino Acids 2004 26(3): 217-233;
11) Ref 12, Table 4;
12) Nilsson, B. O. Inflamm Res. 1999 48(10): 509-515;
13) Li, Y. et al. Nat. Commun. 2013 4: 1760;
14) Le Bail, M. et al. Proc Natl Acad Sci USA 2015 112(2): E204-E213;
15) Papouin, T. and Oliet, S. H. R. Philos Trans R Soc Lond B Biol Sci 2014 369(1654): 20130601;
16) Hardingham, G. E. and Bading, H. Nat Rev Neurosci. 2010 11: 682-696;
17) Parsons, M. P. and Raymond, L. A. Neuron 2014 82(2): 279-293;
18) Coyle, J. T. et al. Neurochem Res. 2020 45(6): 1344-1353;
19) Sason, H. et al. Cereb Cortex 2017 27(2): 1573-1587;
20) Ribeiro, C. S. et al. Brain Res. 2002 929(2): 202-209;
21) Mothet, J.-P. et al. Proc. Natl. Acad. Sci. USA 2005 102(15): 5606-5611

Tables 1, 2, 4, and 8 show transporters, receptors, and enzymes involved in polyamine, GABA and other chemical's creation/degradation reactions, and neurotransmitter signaling, and the various ATS and ITS for these proteins, with many of these proteins depicted in FIG. 10, FIG. 11, FIG. 14, and FIG. 15. FIG. 10 depicts the enzymes involved in polyamine (e.g., spermine, spermidine, putrescine, agmatine), nitric oxide, GABA, and other chemicals (e.g., treatment substances) production and/or degradation. Dotted arrows show reactions that are uncommon/do not occur in mammals, dashed arrows show reactions more common in a mammal's peripheral tissue rather than the reactions likely to occur in the CNS shown by solid arrows. Polyamine(s) (e.g., spermidine, spermine, putrescine, cadaverine, agmatine) are polycationic alkylamines generally found in glial cells. For mice/rats, extracellular polyamine(s) (e.g., spermine, spermidine, putrescine) inhibit Na+ channels. Some intracellular polyamines (e.g., spermidine/spermine) can inhibit the ion channel (e.g., Ca2+ channel) of AMPA-R/kainate-R. Some polyamine(s) (e.g., spermine, spermidine) are released by brain cells into the extracellular space to act activate Glu receptors (e.g., NMDA-R) at low concentrations and inhibit NMDA-Rs at higher concentrations. At high concentrations, polyamines directly activate TRPV1-R with spermine producing the largest response followed by spermidine and putrescine. Agmatine reduces nitric oxide by inhibition of nitric oxide synthases, and nitric oxide inhibits aldehyde dehydrogenase and SAMe decarboxylase. Agmatine promotes production of BDNF. BDNF reduces GAT1 transport (e.g., reuptake) of GABA, by promoting movement of GAT1 from the cell membrane into the cell. The amount of BDNF is also increased by baicalein, butein, fisetin, chrysin, daidzein, Genistein, oroxylin A, quercetin, curcumin, resveratrol, and/or oleuropein (promotes BDNF release from cells). Beta-Ala is an inhibitory neurotransmitter, is produced from polyamine metabolism and activates the GABAA-R/GABAARho-R and the Gly-R (e.g., strychnine-sensitive glycine receptor); and is reuptaken from the extracellular space into cells by GAT2/GAT3/GAT4 [Ref 78, 104, 159, 196, 197, 198, 199, and 200, Table 2; Ref 13, 14, 43, 83, 84, and 85, Table 4; Ref. 12 and 21, Table 8]. FIG. 11 shows a summary of the enzyme pathways from FIG. 10 in the astrocyte (shown at the upper right for FIG. 11) for the transporter movement and enzymatic conversion of chemicals (e.g., treatment substances) in neurons and astrocytes involved in glutamatergic/GABAergic responses. For mice/rats, glial cells' (e.g., Bergmann glial cells, astrocytes) mitochondrial monoamine oxidase B synthesizes GABA from putrescine degradation, and GABA is released by the BEST1 transporter into the extracellular space to bind extrasynaptic GABA receptors (e.g., GABAA-R) on neurons (e.g., glutamatergic granule neurons, GABAergic medium spiny neurons, dopaminergic neurons) for tonic inhibition (e.g., inhibition of postsynaptic neuron's glutamatergic signaling, inhibition of GABA release from presynaptic neurons). Glu/Na+ reuptake by transporter(s) such as EAAT1/EAAT2 promotes release of the GABA by reversing the direction of GABA movement by GAT2/GAT3 to be from the astrocyte into the extracellular space (e.g., near extrasynaptic regions of neurons such as postsynaptic pyramidal glutamatergic neurons) for tonic inhibition of neurons [Ref. 52, 77, 88, 104, 156, 157, 159, 170, 191, 192, 193, 194, and 195, Table 2; Ref. 83, 84, and 85, Table 4; Ref. 6, Table 5; Ref 12, 15, and 22, Table 8].

TABLE 8
Enzyme Reactions for Polyamines and GABA Creation
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Arginine Decarboxylase: (EC 4.1.1.19) [Astrocyte; Mitochondria]

| | |
|---|---|
| S: Arg | P: Agmatine |

Diamine Oxidase: (EC 1.4.3.22)

| | |
|---|---|
| S: Agmatine | P: Gamma-Guanidinobutyraldehyde |

ITS: Aminoguanidine [4, 6, 26]
Aldehyde Dehydrogenase: (EC 1.2.1.3, “ALDHase” e.g., Aldehyde Dehydrogenase A1A)
[Dopaminergic/GABAergic Neuron]

| | |
|---|---|
| S: Gamma-Guanidinobutyraldehyde | P: Gamma-Guanidinobutyrate |

ITS: Ethanol; Gastrodia elata Preparation [Active: Gastrol, Gastrodin,
Bis(4-hydroxybenzyl)sulfide; N6-(4-hydroxybenzyl)adenine Riboside (Possible Inhibitor)] [6]
Ureohydrolase: (EC 3.5.3)

| | |
|---|---|
| S: Gamma-Guanidinobutyrate | P: GABA + Urea |

Arginase: (EC 3.5.3.1)

| | |
|---|---|
| S: Arg | P: Orn + Urea |

ITS: Norvaline [20]
Ornithine Decarboxylase: (EC 4.1.1.17; Cofactor: Pyridoxal 5′ Phosphate) [Neuron; Glial
Cell]

| | |
|---|---|
| S: Orn | P: Putrescine |

ITS: Spermidine/Spermine [Increases Antizyme Amount, and Antizyme Promotes Ornithine
Decarboxylase Degradation]; Oryza sativa Preparation [Active: Spermidine]; Curcumin;
Curcuma longa Preparation [Active: Curcumin] [13, 15, 24]
ATS: Antizyme Inhibitor Enzyme [13, 15]
Agmatinase/Agmatinase-Like Protein: (EC 3.5.3.11) [Neuron; Mitochondria]

| | |
|---|---|
| S: Agmatine | P: Putrescine + Urea |

Diamine Oxidase (EC 1.4.3.22)

| | |
|---|---|
| S: Putrescine | P: 4-Amino-Butanal |

ITS: Aminoguanidine [6]
Aldehyde Dehydrogenase A1A: (EC 1.2.1.3, “ALDHase AlA”) [Neuron, Dopaminergic
Neuron]

| | |
|---|---|
| S: 4-Amino-Butanal | P: GABA |

ITS: Gastrodia elata Preparation [Active: Gastrol, Gastrodin, bis(4-hydroxybenzyl)sulfide,
N6-(4-hydroxybenzyl)adenine Riboside (Possible Inhibitor)]
Putrescine Acetyltransferase: (EC 2.3.1.57) [Bergmann Glial Cell, Astrocyte; Cerebellum]

| | |
|---|---|
| S: Putrescine + Acetyl-CoA | P: N-Acetylputrescine + CoA |

Monoamine Oxidase B: (EC 1.4.3.4; “MAO B”; Cofactor: FAD) [Glial Cell, Bergmann Glial
Cell, Astrocyte; Mitochondria; See Monoamine Oxidase A/B at Table 5]

| | |
|---|---|
| S: N-Acetylputrescine | P: N-Acetyl-4-Amino Butanal |
| S: N-Tele-MethylHistamine | P: t-Methyl-Imidazoleacetic Acid |

ITS: Ethanol; Piper methysticum Preparation [Active: Kavain, Dihydrokavain]; Rx{Ladostigil,
Selegiline, Rasagiline, Lazabemide, Milacemide, Nicotine, Safinamide} [8, 28, 29]
Aldehyde Dehydrogenase 2: (EC 1.2.1.3, “ALDHase 2”) [Bergmann Glial Cell, Astrocyte]

| | |
|---|---|
| S: N-Acetyl-4-Amino Butanal | P: N-AcetylGABA |

ITS: Gastrodia elata Preparation [Active: Gastrol, Gastrodin, Bis(4-hydroxybenzyl)sulfide,
N6-(4-hydroxybenzyl)adenine Riboside (Possible Inhibitor)]
Deacetylase:

| | |
|---|---|
| S: N-AcetylGABA | P: GABA |

Spermidine Synthase: (EC 2.5.1.16) [Neuron; Glial Cell]

| | |
|---|---|
| S: Putrescine + Decarboxylated SAMe | P: Spermidine + 5′-Methylthioadenosine |

Spermine Synthase: (EC 2.5.1.22) [Neuron; Glial Cell]

| | |
|---|---|
| S: Spermidine + Decarboxylated SAMe | P: Spermine + 5′ Methylthioadenosine |

Spermine Oxidase: (EC 1.5.3.16; Cofactor: FAD)

| | |
|---|---|
| S: Spermine | P: Spermidine + 3-Aminopropanal |

Spermidine/Spermine N1-Acetyltransferase: (EC 2.3.1.57)

| | |
|---|---|
| S: Spermine + Acetyl-CoA | P: N1-Acetylspermine + CoA |

ATS: Spermidine/Spermine/Agmatine [Increases Spermidine/Spermine N1-Acetyltransferase
Amount]; Oryza sativa Preparation [Active: Spermidine] [13, 15]
Polyamine Oxidase: (EC 1.5.3.13)

| | |
|---|---|
| S: N1-Acetylspermine | P: Spermidine + 3-Acetamidopropanal |

Spermidine/Spermine N1-Acetyltransferase: (EC 2.3.1.57)

| | |
|---|---|
| S: Spermidine + Acetyl-CoA | P: N1-Acetylspermidine |

Polyamine Oxidase: (EC 1.5.3.13)

| | |
|---|---|
| S: N1-Acetylspermidine | P: Putrescine + 3-Acetamidopropanal |

Aldehyde Dehydrogenase: (EC 1.2.1.3; “ALDHase”)

| | |
|---|---|
| S: 3-Acetamidopropanal | P: N-Acetyl-Beta-Alanine |

ITS: Gastrodia elata Preparation [Active: Gastrol, Gastrodin, Bis(4-hydroxybenzyl)sulfide,
N6-(4-hydroxybenzyl)adenine Riboside (Possible Inhibitor)]
N-Acetyl-Beta-Alanine Deacetylase: (EC 3.5.1.21)

| | |
|---|---|
| S: N-Acetyl-Beta-Alanine | P: Beta-Alanine + Acetate |

Methionine Adenosyltransferase-2Alpha: (EC 2.5.1.6)

| | |
|---|---|
| S: Met | P: SAMe |

SAMe Decarboxylase: (EC 4.1.1.50; “AdoMet Decarboxylase”)

| | |
|---|---|
| S: SAMe | P: Decarboxylated SAMe |

ITS: Spermidine/Spermine [Reduces SAMe Decarboxylase Amount]; Oryza sativa Preparation
[Active: Spermidine]; Agmatine [13]
Nitric Oxide Synthetase: (EC 1.14.13.39; Cofactor: FAD, FMN, Heme, Tetrahydrobiopterin,
Calmodulin)

| | |
|---|---|
| S: 2 Arg + 3 NADPH | P: 2 Nitric Oxide + 2 Citruline + 3 NADPH |

ITS: Agmatine; Terminalia bellirica Preparation [Active: Chebulagic Acid, Chebulinic Acid,
Gallic Acid, Ellagic Acid]; Aminoguanidine [19, 4, 26]
ATS: Increased Calcium/Ca2+ [27]
1) Fiori, L.M. and Turecki G. J Psychiatry Neurosci 2008 33(2): 102-110;
2) Halaris, A. and Piletz, J. 2007 CNS Drugs 21, 885-900;
3) Benitez, J. et al. Metabolism 2018 81: 35-44;
4) Ref. 10, Table 7;
5) Bernstein, H. G. et al. Amino Acids 2011 40: 453-465;
6) Kim, J. I. et al. Science. 2015 350(6256): 102-106;
7) Caron, P. C. et al. Neurochem Int. 1987 10(2): 219-229;
8) Ref. 157, Table 2;
9) Ref. 6, Table 5;
10) Ref. 159, Table 2;
11) Ref. 104, Table 2;
12) Sagar, N. A. et al. Med Sci (Basel) 2021 9(2): 44;
13) Park, M. H and Igarashi, K. Biomol Ther (Seoul) 2013 21(1): 1-9;
14) Khomutov, M. A. et al. Biochemistry (Mosc.) 2013 78(13): 1431-1446;
15) Bae, D.-H. et al. Biochim Biophys Acta Gen Subj. 2018 1862(9): 2053-2068;
16) Ref. 156, Table 2;
17) Ref. 13, Table 4;
18) Ref. 14, Table 4;
19) Ref 12, Table 4;
20) Polis, B. et al. Neural Regen Res. 2019 14(9): 1562-1572;
21) Piletz, J. E. et al. Drug Discov. Today 2013 18(17-18): 880-893;
22) Yoon, B. E. and Lee, C. J. Front Neural Circuits 2014 8: 141;
23) Le-Corronc, H. et al. Mol Neurobiol 2011 44(1): 28-52;
24) Murray-Steward, T. and Casero, R. A. Med Sci (Basel) 2017 5(4): 38;
25) Usharani, P. et al. Clin Pharmacol 2016 8: 51-59;
26) Ref. 12, Table 7;
27) Ref. 4, Table 7;
28) Sarris, J. et al. Aust N Z J Psychiatry. 2011 45(1): 27-35B;
29) Ref. 9, Table 17A

FIG. 12 depicts the transporter mediated movement and enzymatic conversion of chemicals (e.g., treatment substances) in neurons (e.g., glutamatergic neurons) and astrocytes involved in production and release of Glu and GSH upon increasing the levels of Cys/Cys2 in the CNS. The Cys prodrug NAC is hydrolyzed into Cys inside a cell/in the extracellular space. Cys can be converted by oxidation (“OX”) into Cys2. Cys2 is moved into the astrocyte by the Xc- transporter in exchange for moving Glu from the astrocyte into the extracellular space, where Glu may activate a Glu receptor. Activation of a presynaptic neuron's extrasynaptic mGlu2-R/mGlu3-R (“mGlu(2/3)-R”) inhibits adenylyl cyclase that reduces the cAMP amount, inhibits protein kinase A activation, activates K+ channels, and inhibits N-type/voltage-gated Ca2+ channels, resulting in inhibiting the presynaptic neuron's release (shown as a bar in front of a synaptic glutamatergic vesicle) of neurotransmitters (e.g., Glu/Dopamine/D-Ser/Ser) into the synaptic cleft and reduces activation of the postsynaptic neuron's receptors facing the synaptic cleft. For rats, the Xc- antiporter located on glial cells/astrocytes provides about 60% of extrasynaptic Glu in the striatum to bind presynaptic neurons' extrasynaptic axon terminal mGlu(2/3)-R that reduces, by tonic inhibition, synaptic Glu and dopamine release by neurons. The mGlu(2/3)-R were activated by 0.5 μM Glu [Ref. 29, 78, 79, 80, 81, and 82, Table 4; Ref. 10, 12, and 13, Table 3; Ref. 165, Table 2]. It is contemplated that a positive cysteineic may enhance TSF and other sexual functions by promoting the activation of extrasynaptic mGlu(2/3)-R to reduce synaptic Glu release into the synaptic cleft and thus reduce synaptic glutamatergic (e.g., NMDA-R) activity in certain synapses. For mammals, Cys, Glu and Gly are converted into GSH in the astrocyte, and moved into the extracellular space where the GSH may be degraded by enzymes (e.g., EPase) into Gly and Glu neurotransmitters for activation of extrasynaptic NMDA-R (shown on the presynaptic neuron), though Xc- Glu release and/or astrocyte neurotransmitter (e.g., D-Ser) release may also provide coagonist neurotransmitters for extrasynaptic NMDA-R activation. However, extrasynaptic NMDA-R activation often promotes neurotransmitter release (shown at the NMDA-R “ACTIVATION” arrow). At higher levels of Glu (e.g., 500 μM) being released by Xc- after exchange for Cys2, the extrasynaptic mGlu5-R on the postsynaptic neuron becomes activated to activate phospholipase C/protein kinase C and inhibit K+ channels to increase excitatory signaling of the postsynaptic neuron. Gly, Glu and Cys can also be converted to GSH in a neuron (shown in the presynaptic neuron), and GSH/NAC protects proteins from oxidation. Alpha-lipoic acid, described herein, has antioxidant activity that protects GSH from inactivation by oxidation. GSH protection of NMDA-R from oxidation promotes NMDA-R activity to signal for Ca2+ to enter neurons, and Ca2+ promotes release of neurotransmitters by the presynaptic neuron. Cys/Gly (shown in the astrocyte) may inhibit D-Ser production by inhibiting Ser racemase [Ref 10, 11, 12, 13, 14, 15, 16, 17, 18, and 19, Table 3; Ref 164, Table 2; Ref. 126 and 127, Table 4].

Tables 1, 2, 4, and 9A show transporters, receptors, and enzymes that are involved in neurosteroid synthesis/degradation reactions and neurotransmitter signaling, and the various ATS and ITS for these proteins, with many of these proteins depicted in FIG. 13, FIG. 14, and FIG. 15. FIG. 13 depicts the enzymes involved in neurosteroid production and/or degradation, including neurosteroids that are positive allosteric modulators of GABAA-R. For mammals, synaptic GABAA-R promote phasic inhibitory signaling, are found throughout the brain, have low GABA affinity, are activated by benzodiazepines and are activated by neurosteroids; and extrasynaptic GABAA-Rs promote tonic inhibition, and have high GABA affinity and are highly activated by neurosteroids and are not benzodiazepine activated compared to synaptic GABAA-Rs. Allopregnanolone (“APL”), allotetrahydrodeoxycorticosterone, and androstanediol are GABAA-Rs (e.g., extrasynaptic GABAA-R) PAMs that bind a neurosteroid binding site different from GABA, barbiturate, and benzodiazepine sites for tonic and phasic inhibitory neurotransmitter signaling. Some sulfated neurosteroids (e.g., pregnenolone sulfate) inhibit GABAA-Rs. Progesterone, dihydroprogesterone, deoxycorticosterone, dihydrodeoxycorticosterone, testosterone, and dihydrotestosterone bind steroid receptors [Ref. 31, 35, 77, and 76, Table 4]. Finasteride, an inhibitor of 5Alpha-reductase (shown in bold), inhibits the synthesis and degradation of neurosteroids/steroids in several metabolic pathways for various effects on the amounts of different neurosteroids/steroids, though the reduction in GABAA-R PAM synthesis is contemplated as possibly contributing to a reduction in TSF in light of the disclosures herein of activation of GABAA-R as promoting TSF.

TABLE 9A
Enzymes Involved in Neurosteroid Metabolism
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Cholesterol Side-Chain Cleavage Enzyme: (EC 1.14.15.6; “P450scc”) [Astrocyte;
Mitochondria]

| | |
|---|---|
| S: Cholesterol | P: Pregnenolone |

3Beta-Hydroxysteroid Dehydrogenase: (EC 1.1.1.145; “3Beta-HSDase”) [Astrocyte;
Peripheral Tissue]

| | |
|---|---|
| S: Pregnenolone | P: Progesterone |

5Alpha-Reductase: (EC 1.3.1.22) [Astrocyte]

| | |
|---|---|
| S: Progesterone | P: Dihydroprogesterone |

3Alpha-Hydroxysteroid-Oxidoreductase: (EC 1.1.1.50; “3Alpha-HSORase”) [Astrocyte]

| | |
|---|---|
| S: Dihydroprogesterone | P: Allopregnanolone (“APL”) |

Steroid 21-Hydroxylase: (EC 1.14.14.16; “Cytochrome P450c21”)

| | |
|---|---|
| S: Progesterone | P: Deoxycorticosterone |

5Alpha-Reductase: (EC 1.3.1.22) [Astrocyte]

| | |
|---|---|
| S: Deoxycorticosterone | P: Dihydrodeoxycorticosterone |

3Alpha-Hydroxysteroid-Oxidoreductase: (EC 1.1.1.50; “3Alpha-HSORase”) [Astrocyte]

| | |
|---|---|
| S: Dihydrodeoxycorticosterone | P: Allotetrahydrodeoxycorticosterone |
| | (“THDOC”) |

Cytochrome P450 17A1: (EC 1.14.14.19; “P450c17”)

| | |
|---|---|
| S: Pregnenolone | P: 17Alpha-Hydroxypregnenolone |
| S: 17Alpha-Hydroxypregnenolone | P: DHEA |

3Beta-Hydroxysteroid Dehydrogenase: (EC 1.1.1.145; “3Beta-HSDase”) [Astrocyte;
Peripheral Tissue]

| | |
|---|---|
| S: DHEA | P: Androstenedione |

17Beta-Hydroxysteroid Dehydrogenase: (EC 1.1.1.51; “17Beta-HSDase”)

| | |
|---|---|
| S: Androstenedione | P: Testosterone |

5Alpha-Reductase: (EC 1.3.1.22) [Astrocyte]

| | |
|---|---|
| S: Testosterone | P: Dihydrotestosterone |

3Alpha-Hydroxysteroid-Oxidoreductase: (EC 1.1.1.50; “3 Alpha-HSORase”) [Astrocyte]

| | |
|---|---|
| S: Dihydrotestosterone | P: Androstanediol |

Tables 1, 2, 4, 6, 7, 8, 9A and 9B show transporters, receptors, and enzymes involved in GABA, Glu, D-Ser, polyamine, and neurosteroid creation/degradation; and the various ATS and ITS for these proteins, as these transporters, receptors, enzymes are involved in glutamatergic and/or GABAergic neurotransmitter signaling, with many of these proteins depicted in FIG. 9, FIG. 10, FIG. 11, FIG. 12, FIG. 13, FIG. 14, and FIG. 15. For mammals, Leu crossed the BBB into the brain faster than any other amino acid, and branched-chain amino acid aminotransferase (“BCATase”) prefers to catalyze reactions using Ile/Leu over Val as substrate for the creation of new Glu, with between 25% to 50% of amino groups in brain Glu derived from Leu, though some studies indicate Val does contribute much of the new amino group. Val was the last individual BCAA evaluated for altering TSF, due to these general preferences for transport and utilization of Leu over Val, and it was surprising that Val produced a stronger improvement in TSF than either Leu or Ile in a human (i.e., the inventor). Moreover, often combining Val with Leu and/or Ile produced a lesser improvement on TSF than ingesting Val without another BCAA, and it is contemplated that Leu and Ile compete with Val for movement across the BBB, enzymatic conversion into Glu, and/or another mechanism of action. For mice, after Glu release by a glutamatergic neuron, Val, and not Leu or Ile, increased cytoplasmic levels of new Glu that was moved into vesicles and released into the extracellular space. For mice, after repeated treatment with Glu to mimic neurons' repeated synaptic release of Glu into the extracellular space, astrocytes transferred nitrogen from Val, and not Leu or Ile, to increase cytoplasmic levels of new Glu into intracellular Glu, and a Val preferring transaminase may contribute to new Glu production [Ref. 30, Table 6; Ref. 211, Table 2; and Ref. 1, Table 3; Ref. 4, 10, 14, 28, 34, 49, 50, 51, and 52, Table 6; Ref. 1, 68, and 205, Table 2]. As Val promotes improvement in TSF typically more than Leu/Ile, it is contemplated that one or more of these activities of Val promoting Glu cytoplasmic levels after repeated synaptic release in mice may occur in a human as another mechanism of action for Val. For humans, enzymes, including those of the tricarboxylic acid cycle (“TCA cycle,” “Krebs cycle” “citric acid cycle”), catalyze reactions to convert GABA into Glu, and to convert Glu into Gln. For rats and mice, glutamate decarboxylase converts Glu into GABA, and VGAT that moves GABA into a vesicle. Newly synthesized GABA, and possibly newly synthesized dopamine, is preferentially taken into a neuron's synaptic vesicle over preexisting GABA and (possibly) dopamine [Ref. 11, 167, 168, 205, and 210, Table 2; Ref. 10, 28, 34, and 49, Table 6].

FIG. 14 depicts a summary of FIG. 13 and FIG. 10 in the astrocyte (at the upper right) and the general location of various transporter proteins, enzymes, and receptors involved in GABA, Glu, neurosteroid (e.g., allopregnanolone; “APL”), and polyamine production, degradation, release, uptake, and/or receptor binding for neurons and astrocytes. The enzymatic pathway for GABA synthesis in an astrocyte and a GABAergic neuron are similar to astrocytes and glutamatergic neurons shown in FIG. 11 and FIG. 12, though the increased amount of glutamate decarboxylase in a presynaptic GABAergic neuron promotes GABA production for GABAergic synaptic signalling. Transporters, enzymes, and other proteins are bolded and arrows darkened and widened on metabolic pathways that may contribute more to GABA synthesis relative to other metabolic pathways. FIG. 14 depicts a model for mammals, generally based on rodent data and proposed to function similarly in humans, where a BCAA is converted in an astrocyte by mitochondrial branched-chain amino transferase (“BCATaseM”) to Glu then converted to Gln. Gln is released into the extracellular space and moved into a presynaptic neuron (e.g., glutamatergic neuron, GABAergic neuron) where Gln is converted by glutaminase into Glu and Glu converted by glutamate decarboxylase into GABA (e.g., in a GABAergic neuron) for synaptic release. Another possible mechanism for the effect of positive cysteineic (e.g., NAC) is shown as is Glu released from an astrocyte into the extracellular space in exchange for Cys2/Glu released by NAC conversion to Cys/Cys2 and/or GSH degradation in the extracellular space (shown in greater detail in FIG. 12); and the Glu is moved by the presynaptic neuron's Glu transporter(s) (e.g., EAAT2/3) into the neuron for new GABA synthesis. Gly is also shown being in a GABAergic vesicle and released by an astrocyte's GLYT1 transporter, though Gly functioning in inhibitory neurotransmission by activation of a Gly-R (as opposed to functioning in excitatory neurotransmission a NMDA-R coagonist) occurs more in the spinal cord relative to the brain [Ref. 6 and 12, Table 6; Ref. 45, Table 2].

Release of GABA into the synaptic cleft activates post-synaptic GABA receptors (e.g., GABAA-R, GABAB-R) to increase an inhibitory response (i.e., hyperpolarization/reduced depolarization of a neuron's plasma membrane), such as, for example, to reduce the excitatory activity of a postsynaptic neuron (e.g., a glutamatergic neuron). Activation of GABAA-R opens a Cl− channel to allow Cl− to cross a neuron's plasma membrane and enter the neuron to hyperpolarize the neuron for an inhibitory potential. Another ion channel/neurotransmitter receptor may promote or reduce the synaptic inhibitory activity/communication of a neurotransmission signal of a GABAA-R activated ion channel; or another neurotransmitter's synaptic signalling. For example, in a postsynaptic neuron, KCC2 generally acts to depolarize the neuron by movement of Cl− out of a neuron, allowing for inhibitory neurotransmitter responses to occur again when the neuron becomes hyperpolarized again. Activation of TRPV1-R promotes activation of the KCC2 transporter's movement of Cl− K+ to increase inhibitory responses. Activation of GABAB-R activates the G-protein-coupled inwardly rectifying K+ channel (“GIRK”) to promote movement of K+ from the neuron to the extracellular space to promote inhibitory activity/presynaptic GABA release. Activation of a synaptic GABAB-R inhibits adenylyl cyclase to reduce the cAMP amount in the neuron, and reduced cAMP reduces NMDA-R promoted Ca2+ movement into a neuron. Activation of AMPA-R opens a Na+ channel to allow Na+ into a neuron to reduce inhibitory responses by GABAA-R movement of Cl− into the neuron, and inhibition of AMPA-R promotes a GABAA-R's inhibitory responses. Another ion channel/neurotransmitter may promote or reduce the phasic activity of a GABAA-R; or another neurotransmitter's synaptic signalling. For example, for mice, increased tonic GABA overpowers excitatory depolarization thereby increasing inhibitory responses via GABAA-R [Ref. 186, Table 2]. Inhibition of monoamine oxidase B in astrocytes reduces GABA synthesis from putrescine and reduces tonic inhibition by extrasynaptic GABA of neurons (e.g., postsynaptic neurons) [Ref 146, 147, 149, 152, 172, 188, 189, and 190, Table 2]. PAM neurosteroids (e.g., APL) synthesized in astrocytes may be released to promote activation of a postsynaptic neuron's extrasynaptic GABAA-R. For mammals, GABA released from astrocytes BEST1 channel binds GABAA-R/GABAB-Rs to inhibit presynaptic neuron's release of other neurotransmitters (e.g., noradrenaline, serotonin, dopamine) release, such as from monoaminergic/dopaminergic neurons [Ref. 187, Table 2]. GHB is produced from GABA metabolism, and GHB may also be produced by metabolism of ornithine and spermidine. GHB may be transported into synaptic vesicles by the GABA and glycine vesicular transporter; and is an agonist for the GHBR and may be an agonist/modulator for GABAB-R [Ref. 97, Table 4].

For humans, transporters for GABA on astrocytes (e.g., GAT3) reuptake about 20% of GABA from the synaptic cleft (e.g., near GABAergic terminals), with the majority of GABA imported into GABAergic neurons (e.g., presynaptic neurons) that have GAT1 [Ref 16, 72, 76, 179, 180, 181, 182, 183, 184, and 185, Table 2].

FIG. 15 depicts the general location of various transporter proteins, enzymes, and receptors involved in GABA, Glu, production, degradation, release, uptake, and/or receptor binding for neurons and astrocytes, with the location of mitochondrial branched-chain amino transferase (“BCATaseM”) located in BBB endothelial cells for based on human data rather than in astrocytes as based on data generally produced from rats/mice. This model has the enzymatic direction of BCATaseM and cytosolic branched-chain amino transferase (“BCATaseC”) reversed relative to FIG. 14, which can occur as enzymes generally catalyze reactions in both directions [Ref 4, 6, 11, 12, 28, and 53, Table 6]. A difference in the proposed model in FIG. 15 is that a BCAA is predominantly imported into a presynaptic neuron to act as a precursor for Glu, then GABA synthesis, rather than a BCAA being exported leaving Gln being a dominant precursor for Glu, then GABA synthesis, as depicted in FIG. 14. It was contemplated that the relatively weak effect a Gln prodrug, AcetylGln, in improving TSF and other sexual function(s) (See Examples T36:165, T36:167, T36:183, T36:184, T36:185, T36:186, T36:187, T36:191, T38:9) is supportive of the proposed model depicted in FIG. 15.

Bicarbonate (“HCO3−”) produced from carbonic anhydrases (“CAase”) moves through the GABAA-R channel to the extracellular space, possibly in an exchanger reaction, as depicted in the postsynaptic neuron in FIG. 14 and FIG. 15. It was contemplated that as a carbonic anhydrase is typically one of the fastest enzyme's known to man, the activity of a carbonic anhydrase in providing HCO3− for exchange with a Cl− across a GABAA-R may be important in promoting the activity of a GABAA-R; and that ingesting a treatment substance that inhibits a carbonic anhydrase would produce a noticeable counteracting effect to the positive effect on TSR and/or a sexual function that occurs upon ingesting activator of a GABAA-R described herein. Table 9B lists ITS and ATS for various carbonic anhydrases; and as active sites of different carbonic anhydrases are often similar so it is contemplated that a treatment substance described herein as an ITS or ATS of a particular carbonic anhydrase may also be similarly acting on another carbonic anhydrase as well.

TABLE 9B
Carbonic Anhydrase Inhibitors and Activators (EC 4.2.1.1; Catalyzes Substrate:
H2O + CO2 to Product: HCO3— + H+)

Carbonic Anhydrase Type: [Location]
Inhibitor Treatment Substance (“ITS”): Substance [Details (Info)] [Reference No]
Activator Treatment Substance (“ATS”): Substance [Details (Info)] [Reference No]
Carbonic Anhydrase Type: I/ II / III / IV / V / VI / VII / VIII / IX / X /XI / XII / XIV:
I [Cytoplasm; Spinal Cord] / II [GABAergic Neuron; CRP; Oligodendrocyte; Astrocyte;
Cytoplasm] / III [CRP] / IV [Blood-BBB] / V [Mitochondria; Brain; CAase VA in Astrocyte;
Neuron] / VI [Extracellular Space; Brain] / VII [GABAergic Neuron; Cytoplasm] / VIII
[Brain; Neuron] / IX / X [Neuron; Astrocyte] / XI [Neuron; Astrocyte; CRP] / XII / XIV [Cell
Membrane; Brain] [Ref. 35, 36, 37, 38, 39, 40, 41, 43, 44, 45, 46, and 47, Table 6]
ITS: Polyamine [e.g., Spermine, Spermidine; (Binds Active Site Water)]; Oryza sativa
Preparation [Active: Spermidine]; Caffeic Acid; Ferulic Acid
ATS: Histamine; His; D-His; Phe [Active Site Hydrogen-Proton Shuttles to Enhance Reaction
Speed]; D-Phe [Active Site Interactions]
Carbonic Anhydrase Type: I/ II / III / IV / V / VI / VII / VIII / IX / XIV
ITS: Gallic Acid; Ellagic Acid; Quercetin; Silybum marianum Preparation [Active: Quercetin];
Terminalia bellirica Preparation [Active: Gallic Acid, Ellagic Acid]; Coumarin/ThioCoumarin
[Binds Active Site but Not Hydrogen-Proton Donor]; Sulfonamide [Binds Active Site Zinc such
as Rx{Acetazolamide}]
Carbonic Anhydrase Type: I/ II / III / IV / VI / VII / VIII / IX / X / XI / XIV
ITS: Rx{Diazepam}
Carbonic Anhydrase: I / II /-V / VII /VIII / XII
ITS: Hesperidin
Carbonic Anhydrase: I/ II / V / XIV
ITS: Morin
Carbonic Anhydrase: I / II / VA / VII / XIV
ITS: Catechin
Carbonic Anhydrase: I / II / V / IX / XIV
ITS: Apigenin, Matricaria chamomilla Preparation [Active: Apigenin]
Carbonic Anhydrase: I / II / VII
ATS: Rx{Fluoxetine, Sertraline, Citalopram, Sildenafil; SSRI}
Carbonic Anhydrase: I / II / V / VII / IX / XII
ITS: Quercetin-3-Glucoside; Kaempferol; Isorhamnetin
Carbonic Anhydrase: I / II / VA / IX
ITS: Diosmetin; Naringin; Taxifolin; Silybum marianum Preparation [Active: Taxifolin];
6-Aminosaccharin; Eriochtrin [Active Site Zinc Interactions]
Carbonic Anhydrase: I / II
ITS: Syringic Acid; Resveratrol; Polygonum cuspidatum Preparation [Active:
Trans-Resveratrol]; Curcumin; Silymarin; Silybum marianum Preparation [Active: Silymarin]
Carbonic Anhydrase: IV / VII / XII
ITS: Naringenin; Citrus junos Preparation/Citrus paradisi/sinensis Preparation [Active:
Naringenin]; Eriodictyol; Quercetin-3-O-Rhamnoside; Salvianolic acid A; Salvianolic acid B;
Salvia miltiorrhiza Preparation [Active: Salvionolic Acid A, Salvionolic Acid B]
Carbonic Anhydrase: V / VII
ITS: Luteolin; Arachis hypogaea Preparation [Active: Luteolin]; Galangin; Taxifolin; Silybum
marianum Preparation [Active: Taxifolin]; Daidzein; Pueraria mirifica Preparation [Active:
Daidzein]; Puerarin; Pueraria lobata Preparation [Active: Puerarin]; Tiliroside
Carbonic Anhydrase: VII
ITS: Syringic Acid; Resveratrol; Polygonum cuspidatum Preparation [Active:
Trans-Resveratrol]; Curcumin; Silymarin; Silybum marianum Preparation [Active: Silymarin];
Rx{Topiramage}
Carbonic Anhydrase: IV
ITS: Quercetin-3-Glucoside
Carbonic Anhydrase: V
ITS: Endiandrin A; Dihydroguaiaretic Acid [Binds Active Site Water Binding Zinc]; Inhibitor
(CAase VA): Xanthone; Garcinia mangostana Preparation [Active: Alpha-Mangostin (A
Xanthone, Possible Inhibitor)]
Carbonic Anhydrase: XII
ITS: Chrysin; Passiflora genus Preparation [Active: Chrysin]; Kaempferol-3-O-Glucoside

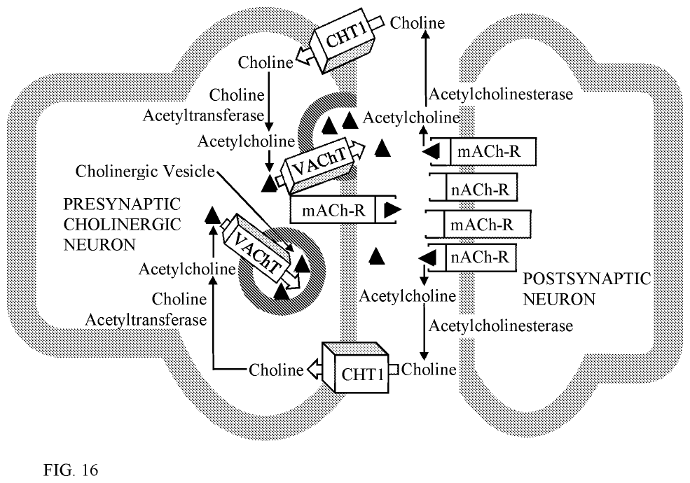

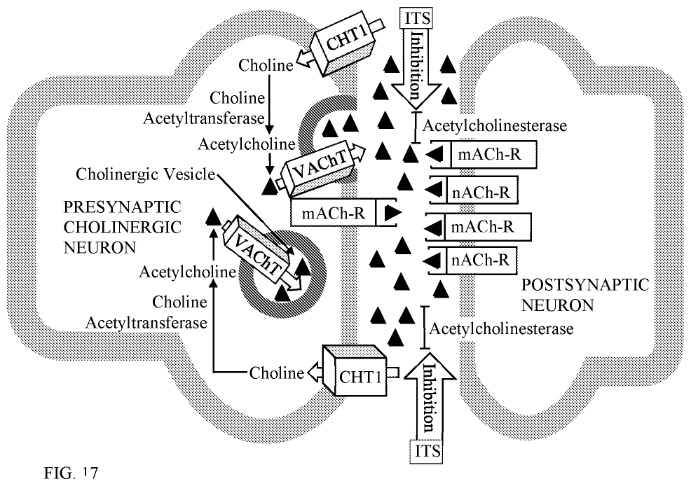

Tables 1, 2, 4, and 10 shows the transporters, receptors, and enzymes that are involved in acetylcholine creation and/or degradation reactions and neurotransmitter signaling, and the various ATS and ITS for these proteins, with many of these proteins depicted in FIG. 16 and FIG. 17. FIG. 16 depicts the general location of various transporter proteins, enzymes, and receptors involved in acetylcholine production, degradation, release, uptake, and/or receptor binding for neurons and astrocytes. FIG. 17 is related to FIG. 16 and depicts the increased accumulation of extracellular acetylcholine that occurs when acetylcholinesterase degradation of acetylcholine is reduced (depicted as a bar in front of acetylcholinesterase) due to an ITS (e.g., Galantamine HBr, Huperzine A).

TABLE 10
Enzyme Reactions Related to Acetylcholine Creation/Degradation
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Acetylcholinesterase: (EC 3.1.1.7)

| | |
|---|---|
| S: Acetylcholine | P: Choline + Acetate |

ITS: Galantamine; Galantamine Hydrobromide; Lycoris radiatia Preparation [Active:
Galantamine Hydrobromide]; Huperzine A; Huperzia serrata Preparation [Active:
Huperzine A]; Gallic Acid; Rosmarinic Acid; Citral; Melissa officinalis Preparation [Active:
Gallic Acid, Rosmarinic Acid, Citral]; Panax genus Preparation [Active: Ginsenoside (e.g.,
Ginsenoside Rg1, Ginsenoside Rb1)]; Polygonum cuspidatum Preparation [Active:
Trans-Resveratrol]; 1,8-Cineole; Eserine; Asiatic Acid; Madecassic Acid; Madecassoside;
Epigallocatechin Gallate; Asiaticoside; Centella asiatica Preparation [Active: Asiaticoside,
Asiatic acid]; Crocus sativus Preparation [Active: Safranal, Crocetin, Dimethylcrocetin];
Trigonelline; Alpha-Pinene; Rosmarinus officinalis Preparation/Satureja myrtifolia
Preparation [Active: Alpha-Pinene]; Alpha-Viniferin; Affinine; Affinisine; Conodurine;
Coronaridine; Corydaline; Corynoline; Cyclanoline; Harmaline; Peganum harmala Preparation
[Active: Harmaline]; Kobophenol A; Lactucopicrin; Taspine; Ungeremine; Ungiminorine;
Caffeine; Coffea arabica Preparation/Coffea robusta Preparation [Active: Caffeine]; Quercetin
[Weak Inhibitor]; Silybum marianum Preparation [Active: Quercetin]; Naringenin [Reduces
Acetylcholinesterase Amount and Activity]; Citrus junos Preparation/Citrus paradisi/sinensis
Preparation [Active: Naringenin]; Rx{Cymserine, Tetrahydrocannabinol, Acotiamide,
Ambenonium, Donepezil, Edrophonium, Ipidacrine, Miotine, Distigmine, Ladostigil,
Neostigmine, Phenserine, Pyridostigmine, Rivastigmine} [1, 2, 3, 4, 5, 6, 7, 9, 13]
ATS: Inhibitor Antidote: Rx{Asoxime Chloride, Obidoxime, Pralidoxime, Trimedoxime
Bromide}
Butyrylcholinesterase: (EC 3.1.1.8)

| | |
|---|---|
| S: Butyrylcholine | P: Choline + Butyric Acid |

ITS: 1,8-Cineole; Alpha-Pinene; Rosmarinus officinalis Preparation/Satureja myrtifolia
Preparation [Active: Alpha-Pinene]; Galantamine; Galantamine Hydrobromide; Lycoris
radiatia Preparation [Active: Galantamine Hydrobromide]; Affinine; Affinisine; Conodurine;
Rx{Cymserine, Ladostigil, Rivastigmine} [1, 2]
Choline Acetyltransferase: (EC 2.3.1.6) [Cholinergic Neuron; Soluble Form in
Intracellular/Extracellular Space, Membrane Bound Form in Plasma/Vesicle Membrane]

| | |
|---|---|
| S: Acetyl-CoA + Choline | P: Acetylcholine + CoA |

ATS: Panax genus Preparation [Active: Ginsenoside (e.g., Ginsenoside Rg1, Ginsenoside
Rb1)]; Alpha-Lipoic Acid [Possible Cofactor] [1]
1) Ref. 10, Table 4;
2) Hostettmann, K. et al. Curr. Org. Chem. 2006 10: 825-847;
3) Ahmed, F. et al. Pharmacogn. Rev. 2013 7: 121-130;
4) Xu, Z.-Q. et al. Cell Biophys. 2012 62(1): 55-58;
5) Li, Y. X. et al. Eur J Drug Metab Pharmacokinet 2007 32(4): 183-187;
6) Haider, S. et al. PLOS ONE 2020 15(1): e0227631;
7) Sriraksa, N. et al. Evid. Based Complement. Altern. Med. 2012 2012: 823206;
8) Sofuoglu, M. and Mooney, M. CNS Drugs. 2009 23(11): 939-952;
9) Jusril, N. A. et al. Molecules. 2020 25(15): 3333;
10) Ref. 162, Table 2;
11) Ref. 148, Table 2;
12) Ref. 9, Table 3;
13) Ref. 8, Table 3

For mammals, a ketogenic diet provides most (e.g., 80% or more) of calories as lipids (e.g., triglyceride fats and oils) degraded by the liver into ketone bodies (e.g., Acetoacetate, Beta-Hydroxybutyrate) that are released into the blood. The blood will have about 0.3 millimolar ketone bodies during a diet that uses glucose for energy, and about 10 mM ketone bodies or more during a ketogenic diet. Blood ketone bodies also increase during fasting after the glucose stored in the liver is depleted. For example, the ketone body beta-hydroxybutyrate amount in the blood increases fivefold during fasting. Ingestion of D-beta-hydroxybutyrate in salt or ester form increases blood ketone levels similar to a ketogenic diet/fasting [Ref 4 and 5, Table 6; Ref 1, Table 3]. The ketone bodies are moved across the BBB by a MCT transporter into the brain to be converted by brain cells into Acetyl-CoA to enter the TCA cycle for energy production. A ketogenic diet may include amino acids that are degraded by energy production metabolic pathways that produce: ketone bodies (preferred “ketogenic amino acids”; e.g., Leu, Lys); ketone bodies and glucose (“ketogenic/glucogenic amino acids” e.g., Val, Ile, Thr, Phe, Met, His, Trp); or glucose (less preferred “glucogenic amino acids” e.g., Ala, Arg, Asn, Asp, Cys, Gln, Glu, Gly, Pro, Ser, Tyr). Increased Acetyl-CoA production by ketone body metabolism may increase the conversion of oxaloacetate into citrate, and the reduced amount of oxaloacetate may reduce the conversion of Glu into Asp and AKG by glutamate oxaloacetate transaminase; thus, allowing more Glu to accumulate in neurons and also be converted into GABA in neurons [Ref 101, Table 2; Ref 1, 6, 12 and 13, Table 11]. Table 11 shows brain alterations associated with a ketogenic diet.

TABLE 11
Brain Alterations Related to a Ketogenic Diet
Treatment Substance [Details]

| | |
|---|---|
| Target | Affect [Reference No] |

Ketogenic Diet

| | |
|---|---|
| Liver | Increased liver ketone release into the bloodstream, increased brain ATP |
| | that is converted into adenosine [2] |
| Adenosine | Reduces Adenosine Kinase Amount; Increased Adenosine Activates the |
| Kinase; A1-R | Adenosine A1-R [5, 9] |
| Neuregulin 1 | Increased Neuregulin 1 Protein Amount that binds the Epidermal Growth |
| | Factor Receptor, and that Increased Presynaptic Neurons′ (e.g., |
| | Interneuron) GABA Amount [4] |
| Brain | Increased GABA, Taurine, Gly, and Ser; Decreased Ala, Phe, Asn, and |
| | Tyr in the Cerebrospinal Fluid [6, 7, 8] |
| Brain | Increased GABA (for most humans) [6, 7, 8] |

Ketogenic Diet [With Medium Chain Fatty Acids/Capric Acid]

| | |
|---|---|
| AMPA-R | Capric Acid Inhibits AMPA-R to Reduce Excitatory Neurotransmission |
| | [9, 10] |

Ketogenic Diet [57% Ketogenic Amino Acids (e.g., Leu, Lys) and 27% Ketogenic/Glucogenic Amino Acids]

| | |
|---|---|
| Pannexin | Pannexin channels of pyramidal neurons released ATP into the |
| Channels; A1-R | extracellular space that was degraded into adenosine A1-R agonist |
| | adenosine to reduce excitation of pyramidal neuron [1] |

12 or 24 Grams of D-Beta-Hydroxybutyrate, in Ester or Salt Form, Increased Blood
Ketone Bodies such as D-Beta-Hydroxybutyrate to 1.0 mM or 2.8 mM, Respectively

| | |
|---|---|
| Blood | Ketogenic effects (e.g., Reduced Blood Glucose) at 1 mM blood |
| | D-Beta-Hydroxybutyrate [6, 7, 8] |

Ketone Bodies [e.g., Acetoacetate, Pyruvate]

| | |
|---|---|
| VGLUT | Ketone bodies (e.g., Acetoacetate) bind VGLUT to prevent allosteric |
| | activation by Cl and reduces VGLUT movement of Glu for release by |
| | neurons (e.g., pyramidal cells), but not astrocytes [3, 9] |

Ketone Bodies [(e.g., Acetoacetate, Beta-Hydroxybutyrate)/LDHase Inhibitors]

| | |
|---|---|
| ATP sensitive K+ | Glycolysis increases cytosolic ATP, and cytosolic ATP inhibits KATP |
| channels (“KATP | channels and activates N+ K+ transporter proteins to reduce |
| channel”)/ | hyperpolarization/promote inhibitory neurotransmission; Ketone bodies |
| Lactate | (which promotes mitochondrial ATP production/reduced cytosolic ATP |
| Dehydrogenase | amount)/LDHase inhibition (which inhibits glycolysis by reducing |
| (“LDHase”) | pyruvate creation and promotes ketogenic adenosine creation) promotes |
| | opening of KATP channel to promote K+ movement out of cells (e.g., |
| | neurons) to increase membrane hyperpolarization (e.g., in GABAergic |
| | neurons)/reduces excitatory neurotransmission [9, 10, 11] |

1) Takeuchi, F. et al. Front Neurosci. 2021 15: 637288;
2) Tescarollo, F. C. et al. J Caffeine Adenosine Res. 2020 10(2): 45-60;
3) Ref. 82, Table 2;
4) Wang, J. et al. Cell Biosci 2021 11(1): 29;
5) Masino, S. A. et al. J Clin Invest 2011 121(7): 2679-2683;
6) Stubbs, B. J. et al. Front Physiol. 2017 8: 848;
7) Dahlin, M. et al. Epilepsy Res. 2005 64(3): 115-125;
8) Wang, Z. J. et al. Magn Reson Med. 2003 49(4): 615-619;
9) Ref. 169, Table 2;
10) Ma, W. et al. J. Neurosci. 2007 27: 3618-3625;
11) Lund, T. M. et al. J Neurochem 2015 132(5): 520-531;
12) Poff, A. M. et al. Front Neurosci. 2019 13: 1041;
13) Yudkoff, M. et al. Annu Rev Nutr. 2007 27: 415-430

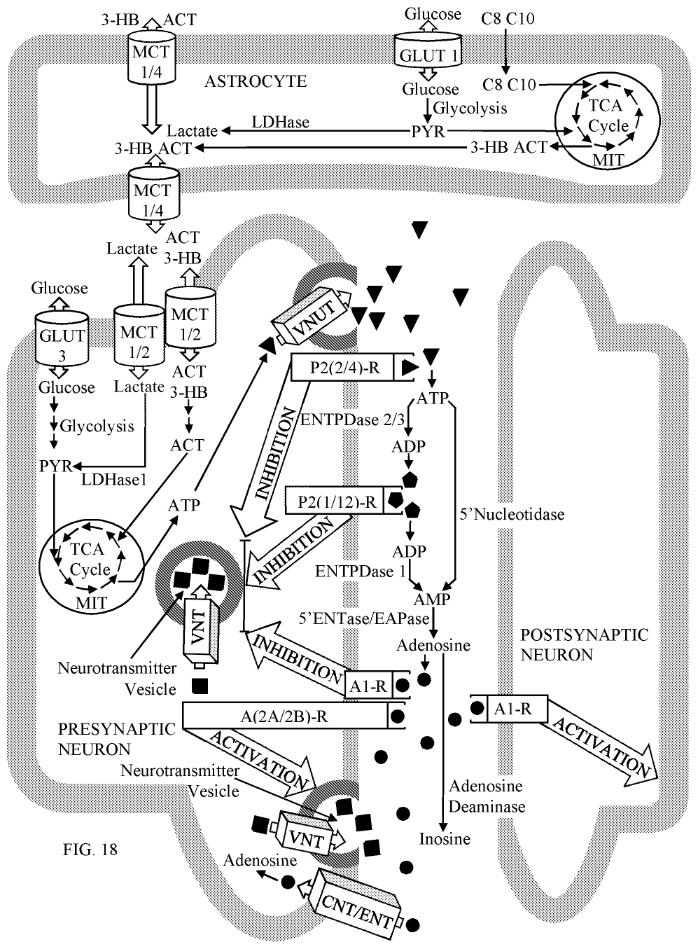

Tables 1, 2, 4, and 12 show the transporters, receptors, and enzymes that are involved in purinergic neurotransmitter (e.g., ATP, ADP, adenosine) synthesis and/or degradation reactions and neurotransmitter signaling, and the various ATS and ITS for these proteins, with many of these proteins depicted in FIG. 18. FIG. 18 depicts the general location of various transporter proteins, enzymes, and receptors involved in purinergic neurotransmitter (e.g., ATP, ADP, adenosine) production, degradation, release, uptake, and/or receptor binding for neurons and astrocytes. Relative to a glucogenic diet, ATP production may be increase by a ketogenic diet where acetoacetate (“ACT”) and beta-hydroxybutyrate (“3-HIB”) are produced in astrocytes [e.g., from degradation of 8-carbon octanoic acid (“C8,” “Caprylic acid”)/10 carbon decanoic acid (“C10,” “Capric acid”) fatty acids]. ACT/3-H1B can also be produced in the liver and/or ingested for transport into an astrocyte, and ACT/3-H1B can also be metabolized by an astrocyte into Gln and released. In contrast, a glucogenic diet promotes glucose release from the liver, conversion of glucose by glycolysis into pyruvation (“PYR”) and conversion of PYR into lactate. In a ketogenic diet, ACT/3-H1B are released by astrocytes and are transported into neurons, and the 3-HB converted into ACT, and ACT used in the mitochondria (“MIT”) for ATP production; while in a glucogenic diet astrocyte lactate release, neurons' lactate/glucose uptake, and then lactate/glucose conversion into PYR for ATP production dominates. The ATP is released as a neurotransmitter that activates purine/pyrimidine receptors [e.g., P2 type 2-R/P2 type 4-R (“P2(2/4)-R”)], and the ATP is then converted into the neurotransmitter ADP that activates purine receptors [e.g., P2 type 1-R/P2 type 12-R (“P2(1/12)-R”], and the ADP then converted into the neurotransmitter adenosine that activates A1-R, and each of these activated receptors on a presynaptic neuron closes Ca2+ channels to inhibit presynaptic neurotransmitter (e.g., acetylcholine, noradrenaline, dopamine, serotonin, Glu, Asp, GABA) release (depicted as a bar in front of a neurotransmitter vesicle). However, adenosine's activation of A2A-R/A2B-R (“A(2A/2B)-R”) on a presynaptic neuron activates presynaptic (e.g., acetylcholine, noradrenaline, dopamine, serotonin, Glu, Gly, Taurine, GABA) neurotransmitter release. For example, for rats, inhibition of adenosine A2A-R inhibits neurons' presynaptic Glu release. In another example, a ketogenic diet/fasting increases extracellular adenosine, and the adenosine reduce dopamine release by A2A-R/D2-R heteromers and A1-R/D1-R heteromers by acting as a dopamine receptor PAM. Activation of A1-R on a postsynaptic neuron opens K+ channels (e.g., K+ ATPase channel) to promote hyperpolarization of the neuron [Ref 101 and 110, Table 2; Ref 8 and 9, Table 4; Ref 2, 3, 8, 9, 10, 11, 12, 13, and 14, Table 12].

For humans, the caffeine is an antagonist of adenosine receptors, and the concentration of caffeine to bind half of the adenosine receptors is about 12 μM for A1-R, 2.4 μM for A2A-R, 13 μM for A2B-R, and 80 μM for A3-R. For humans, caffeine content per cup of coffee [A Coffea arabica Preparation/Coffea robusta Preparation (Active: Caffeine)] is about 25 mg to about 108 mg, and 80 milligrams of caffeine ingested by a 70-kilogram human would produce a brain caffeine content of about 2.6 to about 7.7 μM, with a maximum concentration in the blood about 15 to about 120 minutes after oral ingestion. Caffeine degradation products theophylline and paraxanthine are antagonists of adenosine receptors [Ref. 15, Table 12].

TABLE 12
Purinergic Enzyme Reactions
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Ecto-Nucleoside 5′-Triphosphate Diphosphohydrolase 1: (“ENTPDase 1”) [Cell Surface]

| | |
|---|---|
| S: ATP | P: ADP |
| S: ADP | P: AMP |

Ecto-Nucleoside 5′-Triphosphate Diphosphohydrolase 2: (“ENTPDase 2”) [Cell Surface]

| | |
|---|---|
| S: ATP | P: ADP |

Ecto-Nucleoside 5′-Triphosphate Diphosphohydrolase 3: (“ENTPDase 3”) [Cell Surface]

| | |
|---|---|
| S: ATP | P: ADP |
| S: ADP | P: AMP |

Methionine Adenosyltransferase

| | |
|---|---|
| S: Met | P: SAMe |

Methytransferase

| | |
|---|---|
| S: SAMe | P: SAH |

S-Adenosylhomocysteine Hydrolase: (EC 3.3.1.1)

| | |
|---|---|
| S: SAH | P: Adenosine + Homocysteine |

Ectonucleotidase/5′ Ectonucleotidase: (“5′ENTase”) [Cell Surface]

| | |
|---|---|
| S: ATP | P: Adenosine |

5′-Nucleotidase: (EC 3.1.3.5)

| | |
|---|---|
| S: AMP | P: Adenosine |

Adenosine Deaminase: (EC 3.5.4.4) [Cell Surface/Extracellular Space]

| | |
|---|---|
| S: Adenosine | P: Inosine |

ITS: Progesterone [Reduces Adenosine Deaminase Amount] [2]
Adenosine Kinase: (EC 2.7.1.20) [Brain Cell; Nucleus; Astrocyte]

| | |
|---|---|
| S: Adenosine + ATP | P: AMP + ADP |

Lactate Dehydrogenase: (“LDHase”; e.g., LDHasel, LDHase5) [Neuron; Astrocyte]

| | |
|---|---|
| S: Lactate + NAD+ | P: Pyruvate + NADH |

ITS: Rx{Stiripentol} [5, 6, 7]
1) Ref. 7, Table 5;
2) Fried, N. T. et al. Brain Sci. 2017 7(3): 30;
3) Pascual, O. et al. Science 2005 310(5745): 113-116;
4) Ref. 110, Table 2;
5) Ref. 46, Table 4;
6) Sada, N. et al. Science 2015 347(6228): 1362-1367;
7) Boison, D. Curr Opin Neurol 2017 30(2): 187-192;
8) Thevenet, J. et al. FASEB J. 2016 30(5): 1913-1926;
9) Masino, S. A. and Rho, J. M. Brain Res. 2019 1703: 26-30;
10) Blum, D. et al. J Neurosci 2003 23(12): 5361-5369;
11) Cunha, R. A. J Neurochem. 2016 139(6): 1019-1055;
12) Effendi, W. I. et al Cells 2020 9(3): 785;
13) Butt, A. M. Semin Cell Dev Biol. 2011 22(2): 205-213;
14) Chiang, M. C. et al. Hum Mol Genet. 2009 18: 2929-2942;
15) Fredholm, B. B. et al. Pharmacol Rev 1999 51(1): 83-133

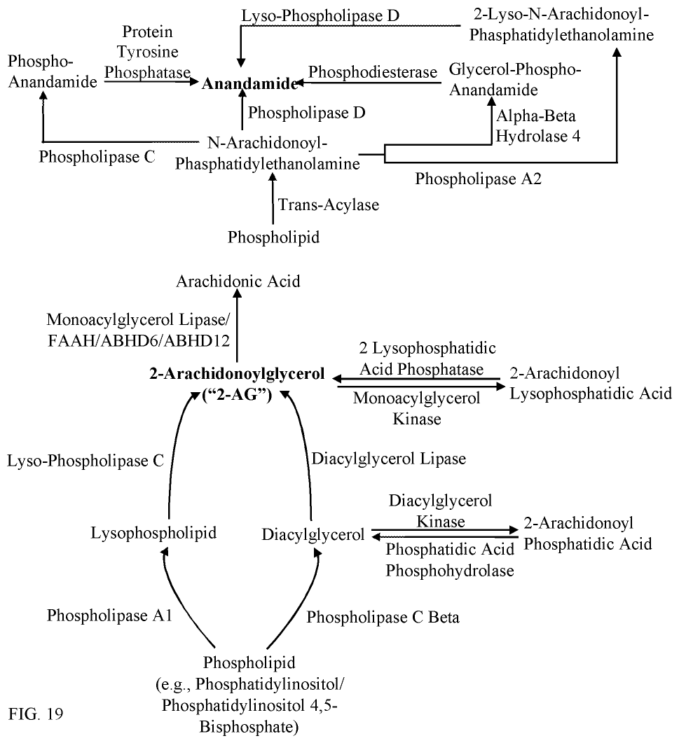

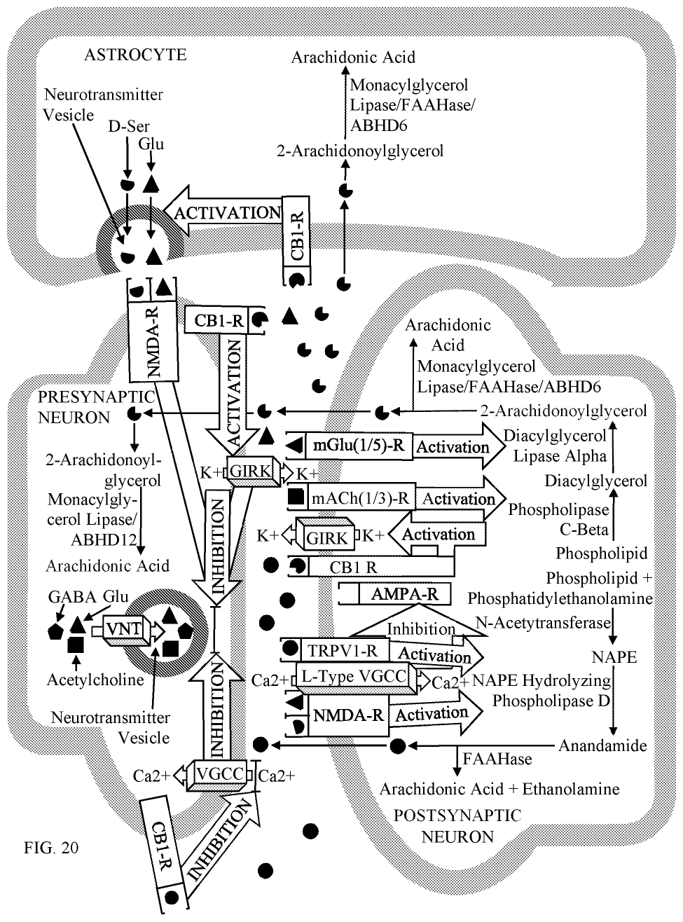

Tables 1, 2, 4, and 13 show transporters, receptors, and enzymes that are involved in cannabinoid creation and/or degradation reactions and neurotransmitter signaling, and the various ATS and ITS for these proteins, with many of these proteins depicted in FIG. 19 and FIG. 20. FIG. 19 depicts the enzymes involved in cannabinoid production and/or degradation, including neurosteroids that are neurotransmitters (e.g., 2-arachidonoylglycerol, anandamide). FIG. 20 depicts the general location of various transporter proteins, enzymes, and receptors involved in cannabinoid neurotransmitter (e.g., 2-arachidonoylglycerol, anandamide) production, degradation, release, uptake, and/or receptor binding for neurons and astrocytes. For mammals, a cannabinoid is a lipid neurotransmitter typically released by a postsynaptic neuron, generally without use of a vesicle (e.g., by diffusion), that is an agonist of plasma membrane and/or mitochondrial cannabinoid receptors [e.g., cannabinoid type-1 receptor (“CB1-R”)] often in the extrasynaptic part of an axon of a presynaptic neuron (e.g., GABAergic neuron, glutamatergic neuron). Nitric oxide and hydrogen sulfide are also produced in a post-synaptic neuron's terminals and diffuse across cellular membranes rather than be released by synaptic vesicles into the extracellular space and/or into other cells. Examples of cannabinoids include 2-arachidonoylglycerol (“2-AG”), and N-arachidonoylethanolamine (“anandamide”). Activation of certain postsynaptic metabotropic receptor(s) [e.g., mACh1-R/mACh3-R (“mACh(1/3)-R”) and/or mGlu1-R/mGlu5-R (“mGlu(1/5)-R”)] activates phospholipase C-Beta/diacylglycerol lipase-alpha to synthesize 2-AG. Activation of a postsynaptic neuron's transient receptor potential vanilloid type 1 receptor (“TRPV1-R”)/NMDA-R/Ca2+ channels (e.g., L-Type VGCC) increases intracellular Ca2+. The postsynaptic neuron's increased Ca2+ activates N-acylphosphatidylethanolamine-hydrolyzing phospholipase D synthesis of anandamide, the Ca2+ activates diacylglycerol lipase-alpha to synthesize 2-AG, and the Ca2+ inactivates the AMPA-R by moving AMPA-R from the cell surface into the cell. Cannabinoid neurotransmitter activation of presynaptic CB1-R/CB2-R inhibits neurotransmitter release by inhibiting voltage-gated Ca2+ channels' (“VGCC”) movement of Ca2+ into presynaptic neurons and activating G-protein-coupled inwardly rectifying K+ (“GIRK”) channels' movement of K+ out of the a presynaptic/postsynaptic neuron that promotes hyperpolarization of the neuron. Activation of CB1-R also inhibits adenylyl cyclase and reduces the cAMP amount and protein kinase A activity. Activation of an astrocyte's cannabinoid receptors (e.g., CB1-R) increases release of neurotransmitters (e.g., D-Ser, Glu) to bind a receptor such as NMDA-R at the extrasynaptic part a presynaptic neuron (e.g., GABAergic neuron, glutamatergic neuron). Though activation of a presynaptic neuron's extrasynaptic NMDA-R often promotes neurotransmitter release, activation of a certain presynaptic neurons comprising some extrasynaptic NMDA-R heteromers (e.g., NMDA-R heteromer less affected by Mg2+) and/or activation of the presynaptic neurons' CB1-R may promote the inhibition of neurotransmitter release from the presynaptic neuron. Inhibition of neurotransmitter release by activation of presynaptic neuron's NMDA-R/CB1-R (via GIRK activation/VGCC inhibition) is depicted as a bar in front of a neurotransmitter vesicle [Ref. 30, 126, and 127, Table 4; Ref. 1, 22, 23, 24, and 25, Table 13].

TABLE 13
Enzyme Reactions Related to Cannabinoids
Enzyme: (EC no; “AAA”; Cofactor) [Location]

| | |
|---|---|
| Substrate for Enzyme Reaction (“S”): | Product of Enzyme Reaction (“P”): |

Inhibitor Treatment Substance (“ITS”): Substance [Details (Info)] [Reference No]
Activator Treatment Substance (“ATS”): Substance [Details (Info)] [Reference No]
Phospholipase C-Beta: (EC 3.1.4.11) [Neuron, Post-Synaptic Neuron]

| | |
|---|---|
| S: Phosphatidylinositol 4,5-Bisphosphate | P: Diacylglycerol + Inositol |
| (“P4,5B”) | 1,4,5-Trisphosphate |

ATS: Activators of Metabotropic Receptors [e.g., mACh1-R/mACh3-R/mGlu1-R/mGlu5-R]
that are Heteromers with Phospholipase C-Beta [1, 15]
Diacylglycerol Lipase-Alpha/Diacylglycerol Lipase-Beta: (EC 3.1.1.116) [Neuron,
Post-Synaptic Neuron]

| | |
|---|---|
| S: Diacylglycerol | P: 2-Arachidonoylglycerol (“2-AG”) + |
| | Fatty Acid |

ATS: Activators of Metabotropic Receptors [e.g., mACh1-R/mACh3-R/mGlu1-R/mGlu5-R]
that are Heteromers with Diacylglycerol Lipase-Alpha; Calcium/Ca2+ [e.g., From Activation of
TRPV1-R/NMDA-R/L-Type VGCC] [1, 2]
2 Lysophosphatidic Acid Phosphatase

| | |
|---|---|
| S: 2-Arachidonoyl Lysophosphatidic Acid | P: 2-Arachidonoylglycerol |

Monoacylglycerol Kinase

| | |
|---|---|
| S: 2-Arachidonoylglycerol | P: 2-Arachidonoyl Lysophosphatidic Acid |

Monoacylglycerol Lipase: (EC 3.1.1.23) [Pre-Synaptic Neuron, Post-Synaptic Neuron]

| | |
|---|---|
| S: 2-Arachidonoylglycerol | P: Arachidonic Acid + Glycerol |

ITS: Monoacyl Glycerol [Competitive Inhibitor] [8]
Alpha-Beta-Hydrolase Domain-Containing 6: (EC 3.1.1.23; “ABHD6”)

| | |
|---|---|
| S: 2-Arachidonoylglycerol | P: Arachidonic Acid + Glycerol |

Alpha-Beta-Hydrolase Domain-Containing 12: (EC 3.1.1.23; “ABHD12”)

| | |
|---|---|
| S: 2-Arachidonoylglycerol | P: Arachidonic Acid + Glycerol |

N-Acyltransferase: (EC 2.3.1) [Neuron, Post-Synaptic Neuron]

| | |
|---|---|
| S: Glycerolphospholipid + | P: N-Arachidonoyl Phosphatidylethanolamine |
| Phosphatidylethanolamine | (“NAPE”) |

N-Acylphosphatidylethanolamine-Hydrolyzing Phospholipase D: (EC 3.1.4.54;
“NAPE-PLD”) [Neuron, Post-Synaptic Neuron]

| | |
|---|---|
| S: N-Arachidonoyl Phosphatidylethanolamine | P: Anandamide (“AEA,′ |
| | “N-arachidonoyl-ethanolamine”) + |
| | 1,2-Diacylglycerol 3-Phosphate |

ATS: Calcium/Ca2+ [e.g., From Activation of TRPV1-R/NMDA-R/L-Type VGCC];
Magnesium/Mg2+; Spermine; Spermidine; Oryza sativa Preparation [Active: Spermidine];
Putrescine [1, 3, 8, 10]
Fatty Acid Amide Hydrolase: (EC 3.5.1.99, “FAAHase”) [Neuron, Post-Synaptic Neuron]

| | |
|---|---|
| S: Anandamide | P: Arachidonic Acid + Ethanolamine |
| S: 2-Arachidonoylglycerol | P: Arachidonic Acid + Glycerol |
| S: Oleamide | P: Oleic Acid |

ITS: Biochanin A; Trifolium pratense Preparation [Active: Biochanin A, Formononetin];
Daidzein; Genistein; Glycine max Preparation [Active: Genistein]; Pueraria mirifica
Preparation [Active: Daidzein, Genistein]; Theobroma cacao Preparation [Active:
N-Oleoylethanolamine, N-linoleoylethanolamine (Inhibit Anandamide Amido-Hydrolase, EC
3.5.1, Degradation of Anandamide)] [4, 5, 6, 9, 20]
Cyclooxygenase 2: (“COX”)

| | |
|---|---|
| S: 2-Arachidonoylglycerol | P: Prostaglandin glycerol esters (e.g., PGE2-G, |
| | PGF2Alpha-G) |

1) Araque, A. et al. Neuropharmacology 2017 124: 13-24;
2) Castillo, P. E. et al. Neuron 2012 76(1): 70-81;
3) Kano, M. et al. Physiol Rev. 2009 89(1): 309-380;
4) Thors, L. et al. Br J Pharmacol. 2010 160(3): 549-560;
5) Thors, L. et al. Br. J. Pharmacol. 2007 150: 951-960A;
6) Ref. 64, Table 4;
7) Ref. 3, Table 4;
8) Ref. 4, Table 4;
9) Thors, L. et al. Br J Pharmacol 2007 152: 744-750B;
10) Liu, Q. et al. Chem. Phys. 2002 Lipids 115: 77-84;
11) Cravatt, B. F. et al. PNAS USA 2001 98: 9371-9373;
12) Ueda, N. et al. Chem Phys Lipids. 2000 108: 107-121;
13) Maccarrone, M. Front Mol. Neurosci. 2017 10: 166;
14) Ref. 30, Table 4;
15) Murataeva, N. et al. Br J Pharmacol 2014 171(6): 1379-1391;
16) Pacher, P. et al. Pharmacol Rev 2006 58(3): 389-462;
17) Ref. 60, Table 4;
18) Maccarrone, M. et al. Nat Rev Neurosci 2014 15(12): 786-801;
19) Ref. 52, Table 4;
20) di Tomaso, E. et al. Nature 1996 382(6593): 677-678;
21) Wei, M. et al. Proc Natl Acad Sci USA 2016 113(19): E2695-E2704;
22) Jensen, K. R. et al. Proc Natl Acad Sci USA 2021 118(4): e2017590118;
23) Del-Bel, E. and De-Miguel, F. F. Front. Synaptic Neurosci. 2018 10: 13;
24) Kaczocha, M. et al. Proc Natl Acad Sci 2009 106(15): 6375-6380;
25) Kaczocha, M. et al. J Biol Chem 2012 287: 3415-3424

As some improvement in TSF was noticed on some days after a poor night's sleep, it was contemplated that changes that occur in the brain after sleep deprivation may identify a possible target for a treatment substance. Treatment substances that promote sleep or wakefulness and the targets of those treatment substances' activity, are shown at Table 14. For mammals, an agonist (e.g., histamine) binding postsynaptic excitatory histamine H1-R promote wakefulness and antagonist/inverse agonist (e.g., diphenhydramine, doxylamine, chlorpheniramine, zolpidem) promote sleep. Antagonist/inverse agonist (e.g., pitolisant) binding the inhibitory histamine H3-R (e.g., neurons that release serotonin, noradrenaline, dopamine, acetylcholine, Glu, and/or GABA) increase the extracellular amount of histamine and other neurotransmitters (e.g., dopamine, serotonin, noradrenaline) and promote wakefulness. For mammals, histidine is moved into neurons and the cerebral spinal fluid by an L-amino acid transporter [Ref. 102, Table 2]. Histamine is removed from the extracellular space mostly by degradation by histamine N-methyltransferase; and some histamine may be moved into astrocytes by organic cation transporter 3 [Ref. 24, Table 5]. A GABAA-R agonist/PAM (e.g., a benzodiazepine site PAM) such as a Valeriana officinalis Preparation/Magnolia genus Preparation promote measures of improved sleep (“promote sleep”); a Piper methysticum Preparation (e.g., affecting a different site than the Benzodiazepine Site) promote sleep; activated GABAB-R receptors on hypocretin/orexin neurons promote sleep; a GABAARho-R antagonist impairs sleep and an agonist such as a Withania somnifera Preparation (Active: withanone, withaferin A, triethylene glycol) promote sleep; and a Passiflora incarnate Preparation which has both GABAA-R/GABAB-R agonist-PAM/antagonist properties promotes sleep. Substances that alter (increase or decrease) GAD/GABA transporters, or decreases GABA transaminase, such as a Melissa officinalis Preparation (decreases GABA transaminase) also promote sleep [Ref. 33, Table 14].

TABLE 14
Brain Alterations Related to Sleep and Wakefulness
Treatment Substance/Method [Sleep Condition/Details]

| | |
|---|---|
| Target for Treatment | Affect [Reference No] |
| Substances (Details) | |

Caffeine [50 μM / about 1.5 Cups of Coffee Ingested After Normal Sleep]

| | |
|---|---|
| A1-R (Antagonist) | Increased Excitatory Post-Synaptic Pyramidal Neurons′ |
| | Activation by Glu Release [4] |

Caffeine [Ingested after Sleep Deprivation]

| | |
|---|---|
| A2A-R (Antagonist) | Attention and Wakefulness Improved [1, 13] |

Caffeine [300 mg Ingested After Normal Sleep]

| | |
|---|---|
| A2A-R/D2-R/D3-R | Increased Antagonist Binding to Dopamine D2-R/D3-R by |
| Heteromers Affected | Allosteric Interaction and/or Increased Dopamine D2-R/D3-R |
| | Cell Surface Expression about 60 to 120 Minutes after Ingestion |
| | [6, 7] |

Sleep Deprivation

| | |
|---|---|
| Adenosine | Extracellular Amount Increase in the Basal Forebrain [22] |
| A1-R | Increased Receptor Concentration and Adenosine Binding to |
| | Receptors [9, 10, 11] |
| A1-R | Decreased Depression that may be due to Adenosine Released |
| | by Astrocytes [31, 32] |
| Inducible Nitric Oxide | Increased Inducible Nitric Oxide Synthase in Neurons (e.g., |
| Synthase | Basal Forebrain, Frontal Cortex), Increased Nitric Oxide and |
| | Extracellular Adenosine Amount [22] |
| Monoamine | Increased Monoamine Neurotransmitters (e.g., Dopamine, |
| Neurotransmitters | Noradrenaline, Adrenaline) in the Brain; (e.g., nucleus |
| | accumbens), Though Serotonin Increased in Some Experiments |
| | (e.g., in the Posterior Hippocampus) and Decreased in some |
| | Experiments (e.g., the Giganeocellular Reticular Nucleus) [23] |
| D2-R/D3-R | Reduced Dopamine D2-R/D3-R in the Ventral Striatum; |
| | Increased Activity in the Thalamus [5, 14] |
| 5-HT1A-R | Likely Reduced Serotonin Receptor Cell Surface Expression |
| | [19] |
| 5-HT2A-R | Likely Increased Serotonin Receptor Cell Surface Expression |
| | [8] |
| Noradrenaline | Orexinergic Neurons Inhibition (Hyperpolarization) Likely |
| | through Increased Noradrenaline Binding/Activation of |
| | Adrenergic Alpha1-R/Alpha2-R [19] |
| AMPA-R | Increased AMPA-R Cell Surface Expression on |
| | Hypocetin/Orexin Neurons (“Orexinergic” Neurons) in Lateral |
| | Hypothalamus [19, 20] |
| GABAA-R | Increased GABAA-R Cell Surface Expression on Cholinergic |
| | Neurons in the Basal Forebrain [19, 21] |
| BDNF | BDNF Concentration Increased in Brain [17, 18] |
| Galanin | Increased Brain Galanin (e.g., Increased in the Hypothalamus) |
| | [29] |

Normal Sleep

| | |
|---|---|
| BDNF | Sleep Promoted [17, 18] |

Ethanol [After Normal Sleep]

| | |
|---|---|
| Adenosine A1-R | Increased Cell Surface Amount of Adenosine A1-R [26] |
| ENT1 Transporter | Inhibits Movement of Adenosine from the Extracellular Space |
| | into Cells [24, 25] |

Adenosine A1-R Agonist

| | |
|---|---|
| Adenosine Al-R (Agonist) | Decreased Depression [31, 32] |

Adenosine Deaminase with Reduced Adenosine Degradation Activity [Normal Sleep]

| | |
|---|---|
| Adenosine Deaminase | Sleep Promoted [27] |

Rx{Tolcapone} [After Sleep Deprivation]

| | |
|---|---|
| Catechol-O-Methyltransferase | Attention Reduced [2] |
| (Inhibitor) | |

Histamine Receptor Inverse Agonist [After Sleep Deprivation]

| | |
|---|---|
| Histamine H3-R, H1-R | Increased Histamine Release into the Extracellular Space; |
| | Increased Wakefulness [3] |

Dopamine Receptor Agonist [DOPA Precursor; After Normal Sleep]

| | |
|---|---|
| Dopamine D2 and/or D3 | Increased Sleepiness [12] |
| (Agonist) | |

Rx{Modafinil/Methylphenidate} [After Sleep Deprivation]

| | |
|---|---|
| DAT (Inhibitor) | Increased Extracellular Dopamine; Increased Wakefulness [12, |
| | 14] |

Lemon Balm Preparation (Active: Citranellal, Geraniol, Rosmarinic acid, Pentacyclic
Ursolic Acid, Oleanolic Acid) [After Sleep Deprivation]

| | |
|---|---|
| GABA Transaminase | Increased GABA [16] |
| (Inhibitor) | |

GHB [After Abnormal Sleep]

| | |
|---|---|
| GABAB-R (Agonist) | Promotes Wakefulness in Narcoleptics [28] |

Galanin [After Normal Sleep]

| | |
|---|---|
| Galanin GalR1-R, GalR2-R | Increased Sleep; Reduced Depression [29, 30] |
| and GalR3-R (Agonist) | |

1) Bodenmann, S. et al. Br J Pharmacol. 2012 165(5): 1904-1913;
2) Valomon, A. et al. Neuropsychopharmacology. 2018 43(7): 1599-1607;
3) James, L. M. et al. Psychopharmacology (Berl). 2011 215(4): 643-653;
4) Kerkhofs, A. et al. Front Pharmacol. 2018 8: 899;
5) Tomasi, D. et al. Transl Psychiatry. 2016 6(5): e828;
6) Ferre, S. et al. Neuropharmacology. 2016 104: 154-160;
7) Volkow, N. D. et al. Transl. Psychiatr. 2015 5(4): e549;
8) Elmenhorst, D. et al. Sleep 2012 35(12): 1615-1623;
9) Longordo, F. et al. Eur J Neurosci 2009 29: 1810-1819;
10) Elmenhorst, D. et al., J Neurosci. 2007 27(9): 2410-2415;
11) Elmenhorst, D. et al. Proc Natl Acad Sci USA 2017 114(16): 4243-4248;
12) Holst, S. C. et al. J Neurosci. 2014 34(4): 566-573;
13) Retey, J. V. et al. Clin Pharmacol Ther. 2007 81(5): 692-698;
14) Volkow, N.D. et al. J Neurosci. 2012 32(19): 6711-6717;
15) Mazzotti, D. R. et al. PLOS One. 2012 7(8): e44154;
16) Cases, J. et al. Med J. Nutrition Metab. 2011 4(3): 211-218;
17) Shi, G. et al. Curr Opin Neurobiol. 2017 44: 43-49;
18) Bachmann, V. et al. Sleep 2012 35(3): 335-444B;
19) Longordo, F. et al. Eur J Neurosci 2009 29: 1810-1819;
20) Rao, Y. et al. J Clin Invest. 2007 117(12): 4022-4033;
21) Modirrousta, M. et al. BMC Neurosci. 2007 8: 15;
22) Kalinchuk, A. V. et al. J Neurochem. 2011 116(2): 260-272;
23) Menon, J. M. L. et al. J Circadian Rhythms. 2019 17: 1;
24) Thakkar, M. M. et al. Alcohol. 2015 49(4): 299-310;
25) Nagy, L. E. et al. J Biol Chem. 1990 265(4): 1946-1951;
26) Ref. 45, Table 4;
27) Bachmann, V et al. Cereb Cortex. 2012 22(4): 962-970A;
28) Brown, R. E. et al. Physiol Rev. 2012 92(3): 1087-1187;
29) Murck, H. et al. J Psychiatr Res 1999 33(3): 225-232;
30) Murck, H. et al. Psychoneuroendocrinology 2004 29(9): 1205-1211;
31) Serchov, T. et al. Neuron. 2015 87(3): 549-562;
32) Hines, D. J. et al. Transl. Psychiatry 2013 3(1): e212;
33) Bruni, O. et al. Nutrients 2021 13(2): 530

### SPECIFIC EXAMPLES

The general effectiveness of various embodiments is demonstrated in the following Examples. The following Examples are provided so that the embodiments might be more fully understood. These Examples are illustrative only and should not be construed as limiting in any way, as other treatment substances that have the same/similar activity of as a treatment substance described herein may be used in combination with a treatment substance described herein and/or as substitute for a treatment substance described herein. For example, it is contemplated that the treatment substances described herein whether listed in the working examples, listed prophetically, and/or being of similar in function to working/prophetic examples as would be understood to one of ordinary skill in the art that they may be used individually and/or combined in various combinations without departing from the scope and spirit of the embodiments of the present invention. Although the invention has been described with reference to several exemplary embodiments, it is understood that the words that have been used are words of description and illustration, rather than words of limitation. Changes may be made within the purview of the appended claims, as presently stated and as amended, without departing from the scope and spirit of the embodiments of the present invention. Although the invention has been described with reference to particular means, materials and embodiments, the invention is not intended to be limited to the particulars disclosed; rather, the invention extends to all functionally equivalent technologies, treatment substances, methods and uses such as are within the scope of the appended claims.

### Example 1: Treatment Substances

Table 15 to Table 33B list various working examples' treatment substances, the vendor's recommended dose (“Dose”), and the vendor; with general categorizations of the treatment substances based on a contemplated dominant mechanism(s) of action, (and additional treatment substance details) for ease of reference. Additional/possible mechanism(s) of action and categorization(s) for the treatment substances are described herein the Detailed Description of the Embodiments/Working Examples (e.g., in the Tables/Figures). Some treatment substances were obtained as powders, crystals, and other loose forms that were placed into capsules [typically noted herein as, for example, “Powder Placed into Capsule”; typically cellulose capsules from Capsule Connection, LLC, 309 Bloom PI, Prescott, AZ 86303, U.S.A; hypromellose capsules from BulkSupplements.com 7511 Eastgate Road Henderson, NY 89011 U.S.A. (“BulkSupps”)] and weighed for ease of oral ingestion of a measured amount of such a treatment substance, though occasionally loose forms of a treatment substance were measured and orally ingested without being placed into a capsule. Some treatment substances were in liquid form when orally ingested. For the Working Examples herein all treatment substances were in capsule form unless otherwise noted in the Tables herein. The inventor's body weight when ingesting the treatment substances varied, and was between about 62 kilograms to about 68 kilograms (i.e., about an average of 65 kilograms) for determining the milligram (“mg”) per kilogram (“Kg”) dosage for each treatment substance as may be applicable for treating others with differing body weights. For example, a low dose of a treatment substance that achieved a desirable A or B score of 9 or more is 100 mg and a high dose is 300 mg then the low dose mg per kilogram would be about 100 mg/68 kilogram (i.e., about 1.47 mg per kilogram) and the high dose mg per kilogram would be about 300 mg/62 Kg (i.e., about 4.84 mg per kilogram). It is contemplated that the mg per kilogram dose of treatment substances and/or the time from ingesting one or more treatment substances may vary from person to person for optimum improvement in treating a sexual dysfunction (e.g., an SRSS including reduced TSF or absent TSF). In some instances, it is contemplated that ingesting one treatment substance may be done at a different time than ingesting another treatment substance to optimize the effect in treating a sexual dysfunction. Any range herein includes any and all sub-ranges and specific values within the cited range, this example provides specific numeric values for use within any cited range that may be used for an integer, an intermediate range, a subrange, a combination of one or more range, and an individual value within a cited range, including in the claims. For example, it is contemplated that the range of dose for a treatment substance of mg per Kg and/or the time in minutes from ingesting the treatment substance to a measurement of a value of TSF/sexual function (e.g., A, B, C, D, scores) may be about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 525%, 550%, 575%, 600%, 625%, 650%, 675%, 700%, 525%, 550%, 575%, 600%, 625%, 650%, 675%, 700%, 725%, 750%, 775%, 800%, 825%, 850%, 875%, 900%, 950%, 1000% or more, relative to a vendor's recommended dose and/or a specific working example of the amount of a treatment substance ingested and/or the time from ingesting a treatment substance to a measurement of TSF and/or another aspect of sexual function. In another example, some treatment substances described herein are preparations from a biological material such as a “4:1” extract indicating that the extract preparation comprises about 4 times the amount of an Active than the original biological material, and doses of preparations from such like biological material may be adjusted according; such as for example, ingesting twice the amount of a 2:1 extract relative to a dose of a 4:1 extract.

TABLE 15
Glu/GABA and/or a Precursor/Prodrug for Glu/GABA
Treatment Substance [Treatment Substance Details]

| | |
|---|---|
| Dose in mg | Treatment Substance Vendor [Notes] |
| (Notes) | |

Valine (“Val”) [See Table 45A]

| | |
|---|---|
| 900 | Manufactured by NutraBio Labs, Inc. 564 Lincoln Blvd., Middlesex, NJ 08846 |
| | U.S.A. (“NutraBio”) |
| 2,000 | BulkSupps [Powder Placed into Capsule] |

Leucine (“Leu”) [See Table 45A]

| | |
|---|---|
| 1,500 | Source Naturals, Inc. P.O. Box 2118, Santa Cruz, CA 95062 U.S.A. (“SNaturals” |

Isoleucine (“Ile”) [See Table 45A]

| | |
|---|---|
| 2,000 | Nutricost LLC 1404 West State Street #208, Pleasant Groove, UT 84062. |
| | (“Nutricost”) |

Norvaline [See Table 45A]

| | |
|---|---|
| 400 | BulkSupps [Powder Put in Capsule] |

BCAAs [Leu, Ile, Val in Same Capsule; See Leu, Ile, Val Above]

| | |
|---|---|
| 650 (Ile) | Now Foods, 395 S. Glen Ellyn Rd. Bloomingdale, IL 60108 U.S.A. (“Now”) |
| 1,600 (Leu) | |
| 650 (Val) | |

BCAA [Micronized Leu, Ile, Val in Same Capsule; See Leu, Ile, Val Above; See Table 45A]

| | |
|---|---|
| 1,250 (Ile) | Manufactured by Optimum Nutrition 975 Meridian Lake Dr. Aurora, IL 60504. |
| 2,500 (Leu) | (“ON Brand”) |
| 1,250 (Val) | |

BCAA [Leu, Ile, Val in Same in Same Tablet; See Leu, Ile, Val Above; See Table 45A]

| | |
|---|---|
| 1,250 (Ile) | Nutrex Research, Inc. 579 South Econ Circle Oviedo, FL 32765 U.S.A. |
| 2,500 (Leu) | (“Nutrex”) |
| 1,250 (Val) | |

BCAA, Cofactors [Leu, Ile, Val, Riboflavin (“RBF”), Pyridoxine (“PYR”) in Same Time
Release Tablet; See Leu, Ile, Val Above; See Table 45A]

| | |
|---|---|
| 550 (Ile) | Manufactured by and Distributed by: Maximum Human Performance, LLC 21 |
| 2,200 (Leu) | Dwight Place, Fairfield, NJ 07004 U.S.A. (“MHP”) |
| 550 (Val) | |
| 1.5 (PYR) | |
| 1.7 (RBF) | |

Ketoisocaproate (“KIC,” “Alpha-Ketoisocaproate”) {Transporter Inhibitor [MCT1/MCT2
Inhibitor]; Positive/Negative Glutamatergic/GABAergic [BCAA Metabolism
Substrate/Product]}

| | |
|---|---|
| 500 | Manufactured by Twin Laboratories, Inc. 2120 Smithtown Ave., Ronkonkoma, |
| | New York 11779 U.S.A. (“Twinlab”) |

Alpha-Ketoglutaric Acid (“AKG”) [See Table 45A]

| | |
|---|---|
| 300 | Manufactured by ProThera ®, Inc. 795 Trademark Drive Reno, NV 89521 U.S.A. |
| | with Klaire Labs ® being a division thereof. (“Klaire Labs”) [1] |

NGABA [See Table 45A]

| | |
|---|---|
| 100 | Antiaging Central LLC 10685 Hazelhurst Drive Houston, TX 77043 U.S.A .; |
| | Manufactured for and Distributed by Relentless Improvement ® LLC P.O. Box |
| | 19220, Reno NV 89511 U.S.A. (“AACentral”) |
| 50 | LifeLink 750 Farroll Rd Unit H, Grover Beach, CA 93433 U.S.A. (“LifeLink”) |

Glutamic Acid (“Glu”; See Table 45A)

| | |
|---|---|
| 500 | Distributed by Swanson Health Products 4075 40th Ave S, Fargo ND 58104 |
| | U.S.A. (“SwansonHP”) |

Glutamine (“Gln”; See Table 45A)

| | |
|---|---|
| 500 | Manufactured for Whole Foods Market 550 Bowie Street Austin TX 78703 |
| | U.S.A. (“WFoods”) |

N-Acetyl L-Glutamine (“AcetylGln”) [Gln Prodrug that Readily Crosses BBB, See Gln Above]
1,000 BulkSupps [Powder Put in Capsule]
GABA [See Table 45A]

| | |
|---|---|
| 750 | Puritan′s Pride, Inc. 4320 Veterans Memorial Hwy, Holbrook, NY 11741 U.S.A. |
| | (“PuritanP”) |

Alanine (“Ala”) {Positive Glutamatergic [Metablic Precursor to Glu; Gly-R/NMDA-R Gly Site
Agonist]; Positive/Negative D-Serinergic [Exhanged for D-Ser by ASCT2]}

| | |
|---|---|
| 500 | Distributed by Montiff, Inc. 7172 Melrose Ave., Los Angeles, CA 90046 U.S.A. |
| | (“Montiff”) |

1) Shank, R. P. and Campbell, G. L. Neurochem Res. 1982 7(5): 601-616

TABLE 16A
Glutaminase Activator
Treatment Substance [Treatment Substance Details]

| | |
|---|---|
| Dose in mg (Notes) | Treatment Substance Vendor [Notes] |

D-Alpha Tocopheryl Succinate (“Tocopheryl Succinate”) [See Table 45A]

| | |
|---|---|
| 329 (267 mg D-Alpha Tocopherol, 62 mg Succinate) | Now |

TABLE 16B
TRPV1-R Activator
Treatment Substance [Treatment Substance Details]

| | |
|---|---|
| Dose in | |
| mg (Notes) | Treatment Substance Vendor [Notes] |

Piper nigrum (“95% PiperineC”) [A Piper nigrum Preparation; Fruit Extract; Active: 95%
Piperine (A “95% Piperine Preparation”; A “Piperine Preparation”); See Table 45A]

| | |
|---|---|
| 10 | Manufactured by Carlyle Nutritionals LLC, 20 Broadhollow Road, |
| | Suite 304 Melville, New York USA (“Carlyle”) |

Vanilla planifolia (“Vanilla”) [A Vanilla planifolia Preparation; (A”Vanilla Preparation,” a
“Vanillin Preparation”) Liquid Bean Extract; Active: Vanillin; See Table 45A]

| | |
|---|---|
| N/A | Adam's Extract & Spice, LLC, 3217 Johnson Road Gonzales, |
| | TX 78629 U.S.A (“Adam's”) |

TABLE 17A
GABA Receptor Agonist/GABA Receptor PAM/Glutamic Acid
Decarboxylase Activator/GABA Transaminase Inhibitor
Treatment Substance [Treatment Substance Details]

| | |
|---|---|
| Dose in | |
| mg (Notes) | Treatment Substance Vendor [Notes] |

Homotaurine [See Table 45A]

| | |
|---|---|
| 100 | Distributed by Smart Nutrition, S.A., 18 Rue de l'Eau, |
| | L-1449, Luxembourg. (“Supersmart”) |

Phenibut HCl (“Phenibut”) {Positive GABAergic [GABAB-R
Full Agonist; Weak GABAA-R Agonist]}

| | |
|---|---|
| 500 | Manufactured by Synaptent LLC. 47 W. Polk St. STE 100-241, |
| | Chicago, IL 60605 U.S.A. (“Liftmode”) [Fine |
| | Crystals in Capsule Unless Otherwise Noted] |

Crocus sativus (“Saffron Extract”) [A Crocus sativus Preparation; (A “Saffron Preparation”);
Equivalent to 88.5 mg (about 89 mg, rounded herein) of Crocus sativus Stigma From 44.25 mg
of 2:1 Stigma Extract; (A “Saffron 2:1 Extract”); the dose below is based on the Equavalent
rather than the weight of the pill/tablet, other extracts herein list the dose by the weight of the
pill/tablet; See Table 45A] [7, 8, 9, 27, 28, 31, 32, 33]

| | |
|---|---|
| 89 | Manufactured by Piping Rock Health Products 2100 Smithtown |
| | Avenue Ronkonkoma, NY 11779 U.S.A. (“PipingRock”) |

Withania somnifera (“Ashwagandha”) [A Withania somnifera Preparation; (An “Ashwagandha
Preparation”); Powder; See Table 45A] [4, 5, 6, 9]

| | |
|---|---|
| 300 | Formulated and Distributed by Pure Nootropics, LLC P.O. Box |
| | 6827 Albuquerque, NM 87197 U.S.A. (“Pure Noops”) |

Boswellia serrata (“Boswellia”) [A Boswellia serrata Preparation; Extract (Oligo Gum Resin);
150 mg Boswellic Acids per 250 mg; See Table 45A]

| | |
|---|---|
| 250 | Manufactured by The Himalaya Drug Company Makali, Makali |
| | Aluru Main Rd, Opp JCB, Bengaluru, Karnataka 562162, India; |
| | Distributed by The Himalaya Drug Company 1101 Gillingham |
| | Lane, Sugar Land, TX 77478 U.S.A. (“Himalaya”) |

Piper methysticum (“Kava”) [A Piper methysticum Preparation; a (“Kava Preparation”); Root
Extract Powder; See Table 45A] [11]

| | |
|---|---|
| 1,000 | BulkSupps [Powder Placed into Capsule] |

Piper methysticum (“Kava Liquid”) [A Piper methysticum Preparation (“Kava Preparation”);
Rhizome with Root Liquid Extract; See “Kava” Above] [11]

| | |
|---|---|
| 580 mg | Herb Farm 20260 Williams Hwy, Williams, OR 97544 U.S.A. |
| (per 0.7 ml) | (“Herb Pharm”) |

Scutellaria baicalensis (“95% Baicalin”) [A Scutellaria baicalensis Preparation; Leaf Extract;
Active: 95% Baicalin (Baicalein Prodrug); a (“Baicalin Preparation”); See Table 45A] [9, 12,
13, 14, 15, 21, 22, 23, 24, 25, 26, 34]

| | |
|---|---|
| 250 | Liftmode [Powder Placed into Capsule] |

Magnolia officinalis (“95% Honokiol/Magnolol”) {A Magnolia officinalis Preparation; A
Magnolia genus Preparation (A “Magnolia Preparation”); Bark Extract; Active: 95%
Honokiol/Magnolol (A “95% Honokiol/Magnolol Preparation”; A “Honokiol/Magnolol
Preparation”); Positive GABAergic [Active: Honokiol/Magnolol (GABAA-R PAM: Not
Neurosteroid/Anesthetic/Ethanol/Picrotoxin Site PAM); Active: 4-O-Methylhonokiol/Obovatol
(GABAA-R Benzodiazepine Site PAM; Increases Alpha1 Subunit Amount)]; Positive/Negative
Cannabinoidergic [Active: 4-O-Methylhonokiol (CB2-R Inverse Agonist/Partial Agonist)] [16,
17, 18, 19, 20]

| | |
|---|---|
| 200 | Liftmode [Powder Placed in Capsule] |

Niacin [NAD Precursor; GABAA-R PAM]

| | |
|---|---|
| 500 | Distributed by H-E-B Central Market 7010 Hwy 71 West |
| | Austin, TX 78735 U.S.A. (“HEB”) |
| 100 | Manufactured by Bluebonnet Nutrition Corporation 12915 |
| | Dairy Ashford Sugar Land, TX 77478 U.S.A. |
| | (“Bluebonnet”) |

Valeriana officinalis (“Valerian”) [A Valerian officinalis Preparation; (A “Valerian
Preparation”); See Table 45A] [1, 2, 3, 9]

| | |
|---|---|
| 500 | WFoods |

Valeriana officinalis [Valerian Root Extract (“VRE”) Standardized to 0.8% Valerenic Acid;
and Valerian Root (“VR”); VRE and VR in Same Capsule; A Valeriana officinalis Preparation;
(A “Valerian Preparation”); See Valerian Above and See Table 45A] [1, 2, 3, 9]

| | |
|---|---|
| 400 | SwansonHP |
| (VRE) | |
| 700 | SwansonHP |
| (VR) | |

Centella asiatica (“Gotu Kola”) {A Centella asiatica Preparation; Leaf Extract, Active:
Minimum 30% Selected Triterpenes; Positive GABAergic [Active: Asiaticoside, Asiatic acid
(Glutamate Decarboxylase Activator); Active: Asiaticoside (GABA Transaminase Inhibitor)];
Positive Cholinergic [Active: Asiaticoside (Acetylcholinesterase Inhibitor)]} [9]

| | |
|---|---|
| 60 | SwansonHP |

Magnesium L-Threonate (“MagT”) [A Magnesium/Mg2+ Preparation; 1 mg Magnesium/Mg2+
per 13.9 mg MagT; See Table 45A]

| | |
|---|---|
| 2,000 | Manufactured by PipingRock, Now, and SNaturals as |
| | vendors) (“Now/PP/SN”) |
| 1,042 | Manufactured for Doctor's Best, Inc. 2742 Down |
| | Ave, Tustin CA 92780 U.S.A. (“DBest”) |

Melissa officinalis (“Lemon Balm”) [A Melissa officinalis Preparation; (A “Lemon Balm
Preparation”); See Table 45A] [9, 29]

| | |
|---|---|
| 1,125 | Manufactured by Oregon's Wild Harvest 1601 NE Hemlock |
| | Ave, Redmond, OR 97756 U.S.A. (“OWH”) |

Melissa officinalis (“Lemon Balm Extract”) [A Melissa officinalis Preparation; (A “Lemon
Balm Preparation”); Extract; Active: 7-15% Rosmarinic Acid, See “Lemon Balm” Above and
See Table 45A] [9, 29]

| | |
|---|---|
| 600 | Manufactured for Nootropics Depot 8380 S. Kyrene Rd. |
| | Ste. 110 Tempe, AZ 85284 U.S.A. (“NoopDepot”) |

Syzygium aromaticum (“Cloves”) [A Syzygium aromaticum Preparation; A “Cloves
Preparation,” “Cloves Extract”); 100 mg 10:1 Flower Bud Extract; See Table 45A]

| | |
|---|---|
| 100 | PipingRock |

Passiflora genus (“Chrysin,” “C”) {[A Passiflora genus Preparation; (A “Passionflower
Preparation”); Flowering Parts Extract, Active: Chrysin; A “Chrysin Preparation”)] and
Piperine (“P,” “95% PiperineS”; (A “95% Piperine Preparation”; A “Piperine Preparation”)
[Active: Piperine in Same Capsule as C; See Table 45A} [9, 35]

| | |
|---|---|
| 500 | SwansonHP |
| (C) | |
| 5 | |
| (P) | |

1) Benke, D. et al. Neuropharmacology. 2009 56(1): 174-181;
2) Andreatini, R. et al. Phytotherapy Res. 2002 16(7): 650-654;
3) Bhattacharyya, D. et al. NMCJ. 2007 9(1): 36-39;
4) Andrade, C. et al. Indian J Psychiatry. 2000 42(3): 295-301;
5) Bansal, P. and Banerjee, S. Pharmacogn Mag 2016 12(46): 121-128;
6) Candelario, M. et al. J Ethnopharmacol. 2015 171: 264-272;
7) Hosseinzadeh, H. and Sadeghnia, H. R. Phytomedicine. 2007 14(4): 256-262;
8) Pitsikas, N. and Tarantilis, P. A. Molecules. 2020 25(23): 5647;
9) Sarris, J. et al. Eur Neuropsychopharmacol. 2011 21(12): 841-860A;
10) Ref. 28, Table 8;
11) Sarris, J. et al. Psychopharmacology (Berl). 2009 205(3): 399-407;
12) Li, J. et al. J Pharml Biomed Anal. 2012 63: 120-127;
13) Zhang, Z. et al. Phytomedicine. 2009 16(5): 485-493;
14) Kuroda, M. et al. Nat Prod Commun. 2012 7(4): 471;
15) Hui, K. M. et al. Biochem Pharmacol 2002 64(9): 1415-1424;
16) Martinez, A. L. et al. J Ethnopharmacol. 2006 106(2): 250-255;
17) Alexeev, M. et al. Neuropharmacology 2012 62(8): 2507-2514;
18) Seo, J. J. et al. Prog Neuropsychopharmacol Biol Psychiatry. 2007 31(7): 1363-1369;
19) Ku, T. H. et al. Phytomedicine. 2011 18(13): 1126-1129;
20) Kalman, D. S. et al. Nutr J. 2008 7: 11;
21) Tarrago, T. et al. Bioorg Med Chem 2002 16 (15): 7516-7524;
22) Liao, J. F. et al. Eur. J. Pharmacol. 2003 464: 141-146;
23) Yu, H. et al., Biochem. Biophys. Res. Commun. 2014 451: 467-472;
24) Zhu, W. et al. Pharm. Biol. 2006 44: 503-510;
25) Zhou, R. et al. Med. Hypotheses 2015 85: 761-764;
26) Liang, W. et al. Aging Dis. 2017 8(6): 850-867;
27) Akao, T. et al. J Pharm Pharmacol 2000 52(12): 1563-1568;
28) Hashemi, M. and Hosseinzadeh, H. Food and Chemical Toxicology 2019 130: 44-60;
29) Pereira, R. P. et al. Ind. Crops Prod. 2014 53: 34-45;
30) Mora, S. et al. J Ethnopharmacol. 2005 97(2): 191-197;
31) Schmidt, M. et al. Wien Med Wochenschr. 2007 157(13-14): 315-319;
32) Hosseinzadeh, H. and Noraei, N. B. Phytother Res. 2009 23(6): 768-774;
33) Ghadrdoost, B. et al. Eur J Pharmacol. 2011 667(1-3): 222-229;
34) Kuroda, M. et al. Nat Prod Commun. 2012 7(4): 471;
35) Grundmann, O. et al. Planta Med. 74: 1769-1773, 2008

TABLE 17B
Glutamic Acid Decarboxylase Inhibitor/GABA Transaminase Activator/
KCC2 Inhibitor/Branched-Chain Amino Acid Aminotransferase Inhibitor
Treatment Substance [Treatment Substance Details]

| | |
|---|---|
| Dose in | |
| mg (Notes) | Treatment Substance Vendor [Notes] |

Humulus lupulus (“Hops”) {A Humulus lupulus Preparation; (A “Hops Preparation”); 10:1
Extract, 1,000 mg Fresh Hops Herb Equivalent Per 100 mg Tablet; Negative GABAergic
[Active: Cohumulone, N-(+)-Adhumulone, Colupulone (Glutamate Decarboxylase Inhibitor)];
Possible Negative Adenosinergic [Possible Antagonist to Caffeine Binding Adenosine-R]}

| | |
|---|---|
| 100 | Amazing Botanicals, Amazingbotanicals.net, 8570 Stirling Rd 102-350, |
| | Hollywood, FL33024 U.S.A. (“ABotanicals”) |

Dehydroepiandrosterone (“DHEA”) {Positive Glutamatergic [NMDA-R PAM; Glutamate
Dehydrogenase Inhibitor; Reduces Glu Degradation]; Negative/Positive GABAergic
[GABAA-R NAM; TRPV1-R Competitive Inhibitor; Reduces Glu Degradation, Glu a GABA
precursor]; Positive Neurosteroidergic [Sigma1-R Activator; Precursor to Testosterone and
Other Steroids/Neurosteroids]; Negative Serotoninergic [Tryptophan Hydroxylase 2 Inhibitor];
Transporter Activator [Increases EAAT2 Transporter Amount]}

| | |
|---|---|
| 25 | SwansonHP |

Brain-Derived Neurotrophic Factor (“BDNF”) {30% Ethyl Alcohol; Liquid; Negative/Positive
GABAergic [Decreases KCC2 Phosphorylation that Decreases KCC2 Activity and Cell Surface
Amount that Reduces GABAergic Signaling; Agonist for Tropomyosin Receptor Kinase B,
Activated Tropomyosin Receptor Kinase B Reduces KCC2 Amount; Reduces GAT1 transport
of Extracellular GABA into Neurons by Promoting Movement of GAT1 from the Cell
Membrane into the Cell]; Transporter Activator [Increases MCT3/4 Amount]} [1, 2, 3]

| | |
|---|---|
| 20 Drops | GUNA, Inc. 3724 Crescent Court West, Whitehall, PA 18052, USA (“GUNA”) |

4′-Dimethylamino 7,8 Dihydroxyflavone (“4-DMA-7,8-DHF”) [Negative GABAergic
(Agonist of Tropomyosin Receptor Kinase B; See BDNF Above)] [2, 3]

| | |
|---|---|
| 8 | NoopDepot [Powder Placed into Capsule] |

Olea europaea (“Olive”) [An Olea europaea Preparation; (An “Olive Preparation”); 400 mg of
Olive Oil Fruit Extract and 300 mg Olive Leaf Extract; Active: 20 mg of
Tyrosol/Hydroxytyrosol/Oleocanthal, Active: 50 mg of Oleuropein, (Promotes BDNF Release
from Cells, see BDNF Above]; Other Active: Maslinic Acid]

| | |
|---|---|
| 700 | The Synergy Company ™ 2279 Resource Blvd, Moab, UT 84532 U.S.A. |
| | (“Synergy”) |

Calcium Beta-Hydroxy Beta-Methylbutyrate Monohydrate (“Beta-Hydroxy
Beta-Methylbutyrate”) [Active: 500 mg Beta-Hydroxy Beta-Methylbutyrate (Reduces Leu
Degradation into KIC by BCATase in Muscle); Active: 80 mg of Calcium/Ca2+ (See Calcium
Pyruvate at Table 45A)

| | |
|---|---|
| 500 | Now |

1) Ref. 241, Table 2;
2) Liu, X. et al. J Med Chem. 2010 53(23): 8274-8286;
3) Rivera, C. et al. J. Cell Biol. 159(5): 747-752, 2002

TABLE 18A
GABA Receptor Antagonist/GABA Receptor NAM
Treatment Substance [Treatment Substance Details]

| | |
|---|---|
| Dose in | |
| mg (Notes) | Treatment Substance Vendor [Notes] |

Pregnenolone {Positive Glutamatergic [Glutamate Dehydrogenase NAM]; Positive/Negative
GABAergic [Reduces Glu Degradation, Glu a GABA Precursor; Precursor to GABAA-R
Neurosteroid PAMs/NAMs]; Positive Glycinergic [Gly-R PAM]; Positive Neurosteroidergic
[Sigma1-R Activator; Precursor of Steroids/Neurosteroids (e.g., DHEA; see DHEA Above]}

| | |
|---|---|
| 10 | SwansonHP |

Pregnenolone (“Preg”; see Pregnenolone Above) and Ascorbyl Palmitate (“AP”) [in Same
Capsule]

| | |
|---|---|
| 30 | Manufactured by Country Life, LLC 180 Vanderbilt Motor Parkway |
| (Preg) | Hauppauge, NY 11788 U.S.A. (“Country Life”) |
| 42 | |
| (AP) | |

Hypericum perforatum (“St. John's Wort”) {A Hypericum perforatum Preparation; (A “St.
John's Wort Preparation”); Extract; Negative GABAergic [Active: Amentoflavone/Bilobalide
(GABAA-R/GABAARho-R NAM)]; Negative Adrenalinergic/Noradrenalinergic [Reduces
Beta-Adrenergic-R Amount]; Positive Monoaminergic/Serotoninergic [Monoamine Oxidase
A/B Inhibitor, Increases Postsynaptic Neuron's 5HT1A-R/5HT2-R Amount]; Positive/
Negative Neurotransmissionergic [Active: Hypericin, Hyperforin (Serotonin/Dopamine/
Noradrenaline/Glu/GABA Reuptake Inhibitor; Glu Release Inhibitor)]; Transporter Activator
[Active: Hyperforin (Pregnana-X Receptor activator); Active: Hyperforin, Hypericin (Hypericin
dose above 1 mg/human/day increased P-GP/BCRP/MRP2 Amount)]; Other Active:
Chlorogenic acid, Quercetin, Quercitrin, Isoquercitrin, Rutin, Hyperoside, Epigenanin
Naphthodianthrone, Pseudohypericin, Adhyperforin, Kaempferol, Luteolin} [2, 3, 4, 5, 6]

| | |
|---|---|
| 600 | BulkSupps [Powder Placed into Capsule] |

Ginkgo biloba (“Ginkgo”) [A Ginkgo biloba Preparation; Leaf Extract, 24% Flavone
Glycosides, 6% Terpene Lactones; See Table 46B] [1, 6]

| | |
|---|---|
| 60 | Formulated by and Manufactured for: MRM, 2665 Vista Pacific Dr., |
| | Oceanside, CA 92056 U.S.A. (“MRM”) |

Dihydromyricetin [See Table 46B]

| | |
|---|---|
| 300 | Pure Noops |

1) Woelk, H. et al. J Psychiatr Res. 2007 41(6): 472-480;
2) Butterweck, V. and Schmidt, M. Wiener Medizinische Wochenschrift. 2007 157(13-14): 356-361;
3) Nathan, P. J. J Psychopharmacol. 2001 15(1): 47-54;
4) Taylor, L. H. and Kobak, K. A. J Clin Psychiatry. 2000 61(8): 575-578;
5) Ref. 116, Table 4;
6) Ref. 9, Table 17A

TABLE 18B
Carbonic Anhydrase Inhibitor
Treatment Substance [Treatment Substance Details]

| | |
|---|---|
| Dose in | |
| mg (Notes) | Treatment Substance Vendor [Notes] |

Apigenin [See Table 46B] [1]

| | |
|---|---|
| 50 | SwansonHP |

Matricaria chamomilla (“Chamomile”) [A Matricaria chamomilla Preparation; (A “Chamomile
Preparation”); Flower Extract; Active: 1.2% Apigenin; See Apigenin Above; Other Active:
Apigenin-7-glucoside, Luteolin, Naringenin, Alpha-Bisabolol] [1]

| | |
|---|---|
| 1200 | Aphrodite's Apothecary LLC, 7933 SW Jack James Drive, Stuart, FL |
| | 34997 U.S.A. (“Barlowe”) |

Matricaria chamomilla (“Chamomile Liquid”) [A Matricaria chamomilla Preparation; (A
“Chamomile Preparation”); Liquid Flower Extract; Active: Apigenin; See Apigenin Above; See
Chamomile Above] [1]

| | |
|---|---|
| 1 mL(not grams) | PipingRock |

Citrus aurantium (“Hesperidin”) [A Citrus aurantium Preparation; (A “Hesperidin
Preparation”); From Plant's Skin; Active: Hesperidin; See Table 46B]

| | |
|---|---|
| 500 | SwansonHP |

Arachis hypogaea (“80% Luteolin”) [An Arachis hypogaea Preparation; (A “Peanut
Preparation”); (A “Luteolin Preparation”); Shell Extract; Active: 80% Luteolin; See Table 46B]

| | |
|---|---|
| 125 | Supersmart |

Garcinia mangostana (“Mangosteen”) [A Garcinia mangostana Preparation; (A “Mangosteen
Preparation”); Peel Extract; Active: 10% Alpha-Mangostin (An “Alpha-Mangostin
Preparation”), 20% Polyphenols; See Table 46B]

| | |
|---|---|
| 500 | SwansonHP |

Terminalia bellirica (“Bahera”) [A Terminalia bellirica Preparation; (A “Bahera Preparation”);
500 mg 20:1 Fruit Extract Equivalent to 10,000 mg of Fruit Powder; Active: Chebulagic Acid,
Chebulinic Acid, Gallic Acid, Ellagic Acid; See Table 46B]

| | |
|---|---|
| 500 | Manufactured by Devki Pharmacy Kakheri Kaithal 136033, Haryana, |
| | India; Manufactured for Amalth Lifecare Private Limited 1st Floor, Shri |
| | Krishan Kripa Complex Paras Cinema Road, Kurukshetra - 136118 India |
| | (“Amalth”) |

Oryza sativa (“1% Spermidine”) [An Oryza sativa Preparation; (A “Rice Preparation”); Rice
Extract: Active: 1% Spermidine (A “Spermidine Preparation”); See Table 46B

| | |
|---|---|
| 300 | Supersmart |

Silybum marianum (“Milk Thistle”) {A Silybum marianum Preparation; (A “Milk Thistle
Preparation”); Seed Extract; 750 mg Seed 4:1 Extract Per Equivalent 3000 mg; A “Milk Thistle
Extract”; Active: Silymarin (Mixture of Silybin, Silybinin, Silichristin, Silidianin, Silimonin,
Isosilichristin, Isosilibinin, Silandrin, Silhermin, Neosilihermin A, Neosilihermin B,
2,3-Dehydrosilibinin, Trisilibinin, Tetrasilibinin, Pentasilibinin), Taxifolin, Quercetin,
Dehydrokaempferol, Linoleic Acid, Oleic Acid, and Palmitic Acid; Negative GABAergic
[Active: Quercetin/Taxifolin/Silymarin (Carbonic Anhydrase Type: I/II/III/IV/V/VA/VI/
VII/VIII/IX/XIV Inhibitor)]; Positive Cholinergic [Active: Quercetin (Weak
Acetylcholinesterase Inhibitor)]; Transporter Inhibitor/Activator [Active: Silymarin/Quercetin
(P-GP/BCRP/MRP1/MRP4/MRP5/MCT1/MCT2/MCT4 Inhibitor); Active: Quercetin (0.1
mg/Kg dose P-GP Activator)]; Active: Quercetin (Positive BDNFic; Increases BDNF
Amount)]; Milk Thistle also Alpha-Glucosidase Inhibitor}

| | |
|---|---|
| 750 | PipingRock |

Pueraria lobata (“Kudzu”) {A Pueraria lobate Preparation; (A “Kudzu Preparation”); Root/Root
Extract; Active: Puerarin; Negative GABAergic [Active: Puerarin (Carbonic Anhydrase: V/
VII Inhibitor)]; Positive BDNFic [Active: Puerarin, Increases BDNF Amount]}
Table 18B: Carbonic Anhydrase Inhibitor
Treatment Substance [Treatment Substance Details]

| | |
|---|---|
| 1226 | Nature's Way Brands, LLC 825 Challenger Dr, Green Bay WI 54311 |
| | U.S.A. (“NaturesWay”) |

1) Ref. 9, Table 17A

TABLE 18C
Carbonic Anhydrase Activator
Treatment Substance [Treatment Substance Details]

| | |
|---|---|
| Dose in | |
| mg (Notes) | Treatment Substance Vendor [Notes] |

D-Phe {Positive GABAergic [Carbonic Anhydrase Type: I/II/III/IV/V/VI/VII/VIII/
IX/X/XI/XII/XIV Activator (Active Site Interactions)]}

| | |
|---|---|
| 500 | DBest |

TABLE 19A
Glutamate Receptor Agonist/Glutamate Receptor PAM
Treatment Substance [Treatment Substance Details]

| | |
|---|---|
| Dose in | |
| mg (Notes) | Treatment Substance Vendor [Notes] |

N-Methyl-D-Aspartic Acid (“NMDA”) [See Table 46B]

| | |
|---|---|
| 100 | Manufactured by Botany Bioscience, PO Box 4010 San Luis Obispo, |
| | CA 93403 USA (“BotanyBio”) |

Aspartic Acid (“Asp”) {Pharmaceutical Grade Asp; Positive Glutamatergic/GABAergic
[Metabolic Precuror to Glu, Glu is Metabolic Precuror to GABA; NMDA-R Glu Site Agonist
(See NMDA Table 46B); Inhibitor of AKG Dehydrogenase Complex (Promotes Increased Glu
Amount)]; Negative D-Serinergic [Serine Racemase Inhibitor]; Transporter Inhibitor/Activator
Promotes AGC1 Glu Movement; SNAT7 Inhibitor]}

| | |
|---|---|
| 500 | SwansonHP |

N,N-DimethylGly HCl (“DimethylGly”) [See Table 45A]

| | |
|---|---|
| 125 | Now |

N-Phenylacetyl-L-Prolyglycine Ethyl Ester (“Noopept”) [See Table 45A] [1, 2]

| | |
|---|---|
| 30 | Health Naturals P.O. Box 10755 Southport N.C. 28461 U.S.A. |
| | (“HealthN”) |

1) Ostrovskaia, R. U. et al. Eksp Klin Farmakol 2002 65(5): 66-72;
2) Vasileva, E. V. et al. Neurochemical J. 2013 7(2): 128-134

TABLE 19B
Glutamate Receptor Antagonist/Glutamate Receptor NAM
Treatment Substance [Treatment Substance Details]

| | |
|---|---|
| Dose in | |
| mg (Notes) | Treatment Substance Vendor [Notes] |

Vitis vinifera (“Grape SeedR,” “GS”) {A Vitis vinifera Preparation; (A “Grape Seed
Preparation”); Active: Seed Extract, comprising 50% Polyphenols; Negative Glutamatergic
[Active: Proanthocyanidin (e.g., Oligomeric Proanthocyanidin at or above 200 mg/Kg)
(NMDA-R/AMPA-R Inhibitor that Inhibits Ca2+ Signaling; mGlu1-R/mGlu5-R Antagonist)]}
and Limon Peel (“Citrus Bioflavonoids,” “CB”) [Active: Citrus Bioflavoniods (Antioxidant);
GS and CB In Same Capsule]

| | |
|---|---|
| 100 | Radiance, LLC 2100 Smithtown Avenue Ronkonkoma, NY |
| (GS) | 11779 U.S.A. (“Radiance”) |
| 30 | |
| (CB) | |

Vitis vinifera (“Grape SeedL,” “GS”) [A Vitis vinifera Preparation; (A “Grape Seed
Preparation”); Active: Seed Extract (See Grape SeedR Above)] and Rosmarinic Acid (“RA”)
[See Lemon Balm Extract Above; GS and RA In Same Capsule]

| | |
|---|---|
| 84 | LifeLink |
| (GS) | |
| 140 | |
| (RA) | |

Glycyrrhiza glabra (“Licorice”) {A Glycyrrhiza glabra Preparation; (A “Licorice Preparation”);
Extract; Active: 25% Glycyrrhizin; Negative Glutamatergic [Active: Isoliquiritigenin
(NMDA-R Antagonist)]; Positive GABAergic [Active: Glabrol, Isoliquiritigenin (GABAA-R
Benzodiazepine Site PAM)]; Transporter Inhibitor [Active: 18Beta-Glycyrrhetinic
Acid/Glycyrrhizin, a 18Beta-Glycyrrhetinic Acid Precursor, (OAT1/OAT3/OAT4 Inhibitor)];
Negative Purinergic [Active: Glycyrrhetinic Acid (VNUT Inhibitor)]}

| | |
|---|---|
| 450 | Jairamdass Khushiram, Plot No - M-14/14-1, MIDC, Taloja, |
| | 410 208 Maharashtra, India (“Bixa”) |

Fasoracetam {Positive/Negative Glutamatergic [mGlu1/2/3/4/5/6/7/8-R Agonist]; Positive
GABAergic [Increases GABAB-R Amount]} [3]

| | |
|---|---|
| 20 | NoopDepot [Powder Placed into Capsule] |

Pramiracetam [See Table 45A] [3, 4]

| | |
|---|---|
| 300 | NoopDepot [Powder Placed into Capsule] |

Theanine [See Table 45A] [2]

| | |
|---|---|
| 100 | Distributed by Jarrow Formulas ® P.O. Box 35994 Los |
| | Angeles, CA 90035-4317 U.S.A. (“Jarrow”) |
| 100 | SwansonHP |
| 250 | BulkSupps [Powder placed into Capsule] |

Theanine and Inositol [in Same Capsule; See Table 45A for Each]

| | |
|---|---|
| 200 | Now |
| (Theanine) | |
| 100 | |
| (Inositol) | |

Dextromethorphan HBr (“Dextromethorphan”) {Softgel; Negative Glutamatergic
[Dextrorphan Prodrug (Dextrorphan NMDA-R Channel Blocker)]; Negative Glycinergic
[Dextromethorphan/Dextrorphan (Gly-R NAM)]; Positive Serotoninergic/Noradrenalinergic/
Dopaminergic [SERT/NET Inhibitor]; Negative Cholinergic [Dextromethorphan/Dextrorphan
(Possible Nicotinic ACh-R with Alpha3/Beta4 Subunit Antagonist/NAM)]; Positive
Neurosteroidergic [Sigma1-R Agonist]}

| | |
|---|---|
| 15 | Made in China, Distributed by Topco Associates LLC, 150 |
| | NW Point Blvd, Elk Grove Village, IL 60007 U.S.A. |
| | (“TopCare”) |

1) Ahn, Seo-Hee et al. BMC Neurosci. 2011 12: 78;
2) Ref. 116, Table 4;
3) Ref. 118, Table 4; 4) Yoshimoto, T. et al. J. Pharmacobiodyn. 1987 10(12): 730-735

TABLE 19C
Glutamate Oxaloacetate Transaminase Activator
Treatment Substance [Treatment Substance Details]

| | |
|---|---|
| Dose in | |
| mg (Notes) | Treatment Substance Vendor [Notes] |

Oxaloacetate {Negative Glutamatergic/GABAergic [Oxaloacetate in Blood (e.g., by Injection)
Reduces Blood Activates Glutamate Oxaloacetate Transaminase to Reduce Glu Levels
Allowing More Movement of Glu Across the BBB from the Brain into the Blood; Glu a GABA
Precursor]} and Ascorbic Acid (“Vitamin C,”) [Antioxidant, Cofactor for Various Enzymes;
Oxaloacetate and Ascorbic Acid in Same Capsule]

| | |
|---|---|
| 100 | Terra Biological LLC, 3830 Valley Centre Drive, Suite |
| (Oxaloacetate) | 705-561, San Diego, CA 92130 U.S.A. (“Terra Bio”) |
| 150 | |
| (Ascorbic Acid) | |

TABLE 20
Xc- Transporter Activator/Positive Cysteineic/Positive GSHic
Treatment Substance [Treatment Substance Details]

| | |
|---|---|
| Dose in | |
| mg (Notes) | Treatment Substance Vendor [Notes] |

N-Acetyl-Cysteine (“NAC.P”) [A “NAC Preparation”; Powder; See NAC at Table 45A]

| | |
|---|---|
| 500 | WFoods |
| 600 | BulkSupps [Powder Placed Into Capsule] |
| 750 | PureBulk, Inc. 1640 Austin Rd, Roseburg, OR 97471 U.S.A. |
| | (“PureBulk”) [Powder Placed Into Capsule] |

N-Acetyl-Cysteine (“NAC.SR”) [A “NAC Preparation”; Sustained and Quick Release Tablet;
See NAC at Table 45A]

| | |
|---|---|
| 600 | Jarrow |

Cysteine (“Cys”) [Pharmaceutical Grade Cys HCl; See Table 45A]

| | |
|---|---|
| 500 | Distributed by Swanson Health Products P.O. Box 2803, |
| | Fargo ND 58108 U.S.A. (“SwansonF1”) |

Glutathione (“GSH”) {Positive/Negative Glutamatergic [Reduces NMDA-R Oxidation to
Promote NMDA-R Activity in a Presynaptic Neuron to Signal for Ca2+ to enter the Neuron
that Promotes Release of Neurotransmitters; Cys/Gly/Glu Precursor (See Cys, Gly, Glu
Herein]; Positive D-Serinergic [Serine Racemase Activator]} [1, 2]

| | |
|---|---|
| 500 | Jarrow |

1) Varga, V. et al. Neurochem Res. 1997 22(9): 1165-1171;
2) Steullet, P. et al. Neuroscience. 2006 137(3): 807-819

TABLE 21
Glycine Receptor Agonist/Glycine Receptor PAM
Treatment Substance [Treatment Substance Details]

| | |
|---|---|
| Dose in | |
| mg (Notes) | Treatment Substance Vendor [Notes] |

Glycine (“Gly”) [See Table 45A]

| | |
|---|---|
| 3,000 | Now |

N-Methyl Glycine (“Sarcosine”) [See Table 45A]

| | |
|---|---|
| 2,000 | Brain Vitaminz, Inc. 6107 SW Murray Blvd, Beaverton, OR 97008 U.S.A. |
| | (“Brainvitamiz”) [Powder Placed in Capsule] |

Trimethylglycine (“Betaine”) [Betaine Anhydrous; See Table 45A]

| | |
|---|---|
| 1,500 | BulkSupps [Powder Placed in Capsule] |

Beta-Alanine (“Beta-Ala”) [See Table 45A]

| | |
|---|---|
| 2,250 | Now |

Atropa belladonna (“Belladonna”) {An Atropa belladonna Preparation; 0.443 mg/Pellet;
Active: Atropine, Scopolamine; Positive Glycinergic [Active: Atropine (Gly-R PAM)];
Negative Cholinergic [Active: Atropine, Scopolamine (mACh-R Antagonist)]}

| | |
|---|---|
| 2.2 | Distributed by Boiron, Inc. 4 Campus Blvd., Newtown Square, PA 19073 USA |
| | (“Boiron”) |

TABLE 22
Polyamine Metabolism GABA Precursor/Polyamine Metabolism Modulator
Treatment Substance [Treatment Substance Details]

| | |
|---|---|
| Dose in | |
| mg (Notes) | Treatment Substance Vendor [Notes] |

Ornithine HCL (“Ornithine”) [See Table 45A]

| | |
|---|---|
| 800 | BulkSupps [Powder Placed into Capsule] |

Phenylethylamine HCl (“Phenylethylamine”) {Positive/Negative Monoaminergic [DAT
Reuptake Inhibitor/VMAT1/2 Inhibitor]; Transporter Inhibitor [PMAT Inhibitor]}

| | |
|---|---|
| 600 | Liftmode [Crystals Placed into Capsule] |

Agmatine Sulfate (“Agmatine”) {Active: Agmatine; (An “Agmatine Preparation”);
Negative/Positive Glutamatergic [NMDA-R Polyamine Site Channel Blocker (Blocks
Votage-Gated Ca2+ Channels); AMPA-R having a GluA1 Subunit Activator (Promotes BDNF
Release); Agmatine Possibly Promotes Glu Synaptic Release (Agmatine moved into
Presynaptic Neuron and into Synaptic Vesicle, often with Glu, for Corelease)]; Positive
Cholinergic [Nicotinic ACh-R Agonist]; Positive Serotoninergic [5HT2A-R/5HT3-R Agonist];
Positive Noradrenalinergic/Adrenalinergic/Imidazolinergic [I1-R/I2-R/Alpha2-R Agonist];
Negative Nitroergic [Neuronal Nitric Oxide Synthase Inhibitor]; Positive Monoaminergic
[Monoamine Oxidase Inhibitor]; Positive/Negative Polyaminergic/Polyamineic [SAMe
Decarboxylase Inhibitor; Increases Spermidine/Spermine Acetyltransferase Amount];
Transporter Inhibitor/Activator [OCT1/OCT2 Inhibitor; VPAT Activator; Ornithine
Decarboxylase Inhibitor (Via Activating Antizyme); Polyamine Uptake Inhibitor
(Competitively Inhibits Polyamine OCT1/2/3 Transporters)]; Positive BDNFic [Promotes
BDNF Production]}

| | |
|---|---|
| 750 | BulkSupps [Powder Placed into Capsule] |

TABLE 23A
D-Serine/Positive D-Serineic/Serine Racemase Activator
Treatment Substance [Treatment Substance Details]

| | |
|---|---|
| Dose in | |
| mg (Notes) | Treatment Substance Vendor [Notes] |

D-Serine (“D-Ser”) [See Table 45A]

| | |
|---|---|
| 2,800 | Manufactured for and Distributed by Relentless Improvement ® LLC |
| | P.O. Box 19220, Reno NV 89511 U.S.A. (“Relentless”) |

Calcium Pyruvate [179.8 mg Calcium/Ca2+ to 719.2 mg Pyruvate Ratio; See Table 45A]

| | |
|---|---|
| 2,000 | BulkSupps [Powder Placed Into Capsule] |

TABLE 23B
Serine Racemase Inhibitor
Treatment Substance [Treatment Substance Details]

| | |
|---|---|
| Dose in | |
| mg (Notes) | Treatment Substance Vendor [Notes] |

Collagen Peptides [A “Collagen Preparation”; Tablets of Amino Acids Crushed into Powder
and Placed into Capsule; 10.3% Hydroxyproline; 1.1% Hydroxylysine; 6.1% BCAA; See Table 45A]

| | |
|---|---|
| 368 | Manufactured by Vital Proteins LLC 3400 Wolf Road Ste. 200 |
| | Franklin Park, IL 60131 U.S.A. (“Vital Proteins”) |

TABLE 24
Acetylcholinesterase Inhibitor/Acetylcholine Receptor
Agonist/Acetylcholine Precursor/Positive Acetylcholineic
Treatment Substance [Treatment Substance Details]

| | |
|---|---|
| Dose in | |
| mg (Notes) | Treatment Substance Vendor [Notes] |

Lycoris radiatia (“Galantamine HBr”) [A Lycoris radiatia Preparation; A “Galantamine
Preparation”; Root Extract; Active: Galantamine Hydrobromide; See Table 45A]

| | |
|---|---|
| 4 | Relentless [Tablet] |

Huperzine A [See Table 45A]

| | |
|---|---|
| 0.05 | SwansonHP |

Panax ginseng C. A.Mey. (“Panax ginseng”) [A Panax genus Preparation; A Panax ginseng
Preparation; Root Extract; Active: At least 5% Ginsenoside; See Table 45A] [1, 3]

| | |
|---|---|
| 1,000 | BulkSupps [Powder Placed in Capsule] |

Polygonum cuspidatum (“50% Trans-Resveratrol”) [A Polygonum cuspidatum Preparation; A
“Trans-Resveratrol Preparation”; Root Extract; Active: Trans-Resveratrol (250 mg per 500 mg);
See Table 45A]

| | |
|---|---|
| 500 | WFoods |

Alpha-GlyceroPhosphoCholine [See Table 45A]

| | |
|---|---|
| 300 | SwansonHP |

Citicoline Sodium (“Citicoline”) [99% Pure; (Choline Prodrug/Metabolized into Choline)] [2]

| | |
|---|---|
| 250 | Liftmode [Powder Placed into Capsule] |

Centrophenoxine [See Table 45A]

| | |
|---|---|
| 500 | HealthN |

Uncaria tomentosa (Bark; “Cat's Claw Bark,”“CCB”) and [Bark Extract; “Cat's Claw Bark
Extract,” “CCBE”) [A Uncaria tomentosa Preparation; (A “Cat's Claw Preparation”); CCB and
CCBE in the Same Capsule; 4% Alkaloids, 7 mg per 175 mg Bark Extract; See Table 45A]

| | |
|---|---|
| 160 | |
| (CCB) | |
| 175 | Natures Way |
| (CCEB) | |

Uncaria rhynchophylla (Dry Stalk Extract; “Cat's Claw Stalk”) [A Uncaria rhynchophylla
Preparation; (A “Cat's Claw Preparation”); 983 mg per mL; See Table 45A]

| | |
|---|---|
| 983 | Hawaii Pharm LLC, 501 Sumner St, Honolulu HI 96817 USA |
| | (“HawaiiP”) |

1) Dang, H. et al. Prog Neuropsychopharmacol Biol Psychiatry. 2009 33(8): 1417-1424;
2) Secades, J. J. and Lorenzo, J. L. Methods Find Exp Clin Pharmacol 2006 28(Suppl B): 1-56;
3) Ref. 9, Table 17A

TABLE 25A
Fatty Acid Amide Hydrolase Inhibitor
Treatment Substance [Treatment Substance Details]

| | |
|---|---|
| Dose in | |
| mg (Notes) | Treatment Substance Vendor [Notes] |

Glycine max (“80% Genistein”) {A Glycine max Preparation; (A “Genistein Preparation”);
Isoflavone Extract, Active: 80% Genistein; Positive/Negative GABAergic [Active: Genistein
(GAT1 Inhibitor, GABAA-R Antagonist, Decreases KCC2 Phosphorylation that Decreases
KCC2 Activity)]; Negative Glutamatergic [Active: Genistein (NMDA-R Inhibitor)]; Negative
Glycinergic [Active: Genistein (Gly-R Antagonist)]; Negative Seratoninergic/Dopaminergic/
Noradrenalinergic/Adrenalinergic/Catecholaminergic/Melatoninergic [Active: Genistein
(Aromatic L-Amino Acid Decarboxylase Inhibitor)]; Positive Cannabinoidergic [Active:
Genistein (Fatty Acid Amide Hydrolase Inhibitor)]; Transporter Inhibitor [Active: Genistein
(P-GP/BCRP/MRP1 Inhibitor)]; Positive BDNFic [Active: Genistein (Increases BDNF
Amount)]}

| | |
|---|---|
| 250 | Manufactured by Vital Nutrients 45 Kenneth Dooley Drive |
| | Middletown, CT 06457 U.S.A. (“VitalN”) |

Oleamide [Granules Loose in Hand When Ingested Unless Otherwise Noted; See Table 45A]

| | |
|---|---|
| 50 | Liftmode |

Palmitoylethanolamide [Positive Cannabinoidergic (Enhances Anandamide Activity; Fatty
Acid Amid Hydrolase Competitive Substrate)]

| | |
|---|---|
| 1200 | Supersmart |
| 400 | NoopDepot |

Trifolium pratense (“Red Clover”) {A Trifolium pratense Preparation; (A “Red Clover”
Preparation”); Extract; Active: 20% Isoflavones (e.g., Biochanin A, Formononetin); Positive
Cannabinoidergic [Active: Biochanin A, Formononetin (Fatty Acid Amid Hydrolase
Inhibitor)]; Transporter Inhibitor [Active: Biochanin A, Formononetin (P-GP/BCRP/MRP1/
MCT1 Inhibitor)]}

| | |
|---|---|
| 400 | Manufactured by KAN Phytochemicals Pvl. Ltd, 1390, HSIIDC Industrial Estate |
| | Rai, Rai, Sonipat 131029, Haryana, India for Maple Life Sciences A-50, III Floor, |
| | Saraswati Vihar, Pitampura, New Delhi 110034, India (“KAN”) |

TABLE 25B
Cannabinoid Receptor Activator
Treatment Substance [Treatment Substance Details]

| | |
|---|---|
| Dose in | |
| mg (Notes) | Treatment Substance Vendor [Notes] |

6% Beta-Caryophyllene {A Piper nigrum Preparation; (A “Beta-Caryophyllene Preparation”);
A Fruit Extract; Positive Cannabinoidergic [Active: 6% Beta-Caryophyllene (CB2-R Agonist)];
Other Possible Active: Piperine (See Piperine Herein) }

| | |
|---|---|
| 500 | NoopDepot |

TABLE 26A
Nitric Oxide Synthase Inhibitor
Treatment Substance [Treatment Substance Details]

| | |
|---|---|
| Dose in | |
| mg (Notes) | Treatment Substance Vendor [Notes] |

Aminoguanidine HCl (“Aminoguanidine”) {Negative Nitroergic [Nitric Oxide Synthase
Inhibitor]; Negative GABAergic [Diamine Oxidase Inhibitor (Reducing Polyamine Metabolic
Conversion to GABA)]}

| | |
|---|---|
| 300 | Supersmart |

TABLE 26B
Positive Nitric Oxideic
Treatment Substance [Treatment Substance Details]

| | |
|---|---|
| Dose in | |
| mg (Notes) | Treatment Substance Vendor [Notes] |

Epimedium sagittatum (“98% Icariin”) [An Epimedium sagittatum Preparation; (An “Icariin
Preparation”); Leaf Extract; Active: 98% Icariin; See Table 45A and See Table 46B]

| | |
|---|---|
| 50 | Liftmode [Powder Placed Into Capsule] |

TABLE 27A
Histamine Receptor Inhibitor
Treatment Substance [Treatment Substance Details]

| | |
|---|---|
| Dose in | |
| mg (Notes) | Treatment Substance Vendor [Notes] |

Meclizine HCl (“Meclizine”) {Tablet; Negative Histaminergic [Histamine H1-R Antagonist];
Negative Cholinergic [mACh-R Antagonist; Also 51 mg Calcium/Ca2+ Per Tablet (See
Calcium Pyruvate at Table 45A)]}

| | |
|---|---|
| 13 | Reliable 1 Laboratories LLC 99 West Hawthorne Avenue Suite |
| | 610 Valley Stream, NY 11580 U.S.A. (“Reliable”) |
| | [Tablet Crushed to Powder and Put into Capsule] |

Chlorpheniramine Maleate (“Chlorpheniramine”) {Tablet; Negative Histaminergic [H1-R
Inverse Agonist]; Negative Cholinergic [mACh-R Antagonist]; Positive Serotoninergic
[SSRI/SERT Inhibitor]}

| | |
|---|---|
| 4 | Distributed by Walmart, Inc. 702 SW 8^{th} St, Bentonville, |
| | AR 72716 USA (“Walmart”) |

Diphenhydramine HCl (“Diphenhydramine”) {Tablet; Negative/Positive Histaminergic [H1-R
Inverse Agonist; Histamine N-Methyltransferase Inhibitor]; Negative Cholinergic [mACh-R
Antagonist]; Positive Serotoninergic [SSRI/SERT Inhibitor]; Sodium Channel Blocker}

| | |
|---|---|
| 25 | Walmart |

TABLE 27B
Monoamine Precursor/Monoamine Prodrug/Monoamine Degradation Inhibitor
Treatment Substance [Treatment Substance Details]

| | |
|---|---|
| Dose in | |
| mg (Notes) | Treatment Substance Vendor [Notes] |

Griffonia simplicifolia (“5HTP”) [A Griffonia simplicifolia Preparation; A (“5HTP
Preparation”); Seed Extract, Active: 5-Hydroxytryptophan; See Table 45A]

| | |
|---|---|
| 100 | Bluebonnet |

Tryptophaan (“Trp”) [See Table 45A]

| | |
|---|---|
| 500 | WFoods |

Phenylalanine (“Phe”) {Positive/Negative Dopaminergic [D1-R/D5-R/D2-R/D3-R/D4-R
Agonist; Tyrosine Hydroxylase Inhibitor; Aromatic L-Amino Acid Decarboxylase Competitive
Substrate]; Positive/Negative Adrenalinergic/Noradrenalinergic [Alpha1-R/Alpha2-R/Beta1-R
Agonist; Aromatic L-Amino Acid Decarboxylase Competitive Substrate]; Positive
Phenethylaminergic [Phenethylamine Precursor]; Negative Serotoninergic/Catecholaminergic
[Aromatic L-Amino Acid Decarboxylase Competitive Substrate]; Negative/Positive
Glutamatergic/GABAergic [Competitive Inhibitor BCAA movement by LAT1; Carbonic
Anhydrase Type: I/II/III/IV/V/VI/VII/VIII/IX/X/XI/XII/XIV Activator (Active
Site Hydrogen-Proton Shuttles to Enhance Reaction Speed)]; Readily Crosses the BBB from
Blood into the Brain}

| | |
|---|---|
| 500 | Now |

Tyrosine (“Tyr”) [See Table 45A]

| | |
|---|---|
| 500 | WFoods Vendor. HEB Alternative Vendor |

N-Acetyl L-Tyrosine (“AcetylTyr”) [Tyr Prodrug; See Table 45A] [1]

| | |
|---|---|
| 400 | BulkSupps [Powder Placed into Capsule] |

Mucuna Pruriens [A Mucuna Pruriens Preparation; Seed Extract; Active: L-DOPA
(“DOPA”); See Table 45A]

| | |
|---|---|
| 500 | BulkSupps [Powder Put into Capsule] |

Histidine (“His”) {Positive Histaminergic [Histamine Precursor (Histamine/His
H1-R/H2-R/H3-R/H4-R Agonist)]; Positive Glutamatergic/GABAergic [His Precursor to Glu;
His/Histamine Carbonic Anhydrase Type: I/II/III/IV/V/VI/VII/VIII/IX/X/XI/XII/
XIV Activator]; Transporter Inhibitor [His (SNAT1/SNAT 2/SNAT 3/SNAT 5/PROT
Inhibitor); Histamine (PMAT/VPAT Inhibitor)]; Negative Glutamatergic [Histamine Channel
Blocker for NMDA-R with NR2A/NR2B Subunit But PAM when Gly Concentration Low];
Negative Tryptophanic [His Activates Tryptophan Dioxygenase (See Trp Above)]}

| | |
|---|---|
| 500 | SwansonHP |

Zanthoxylum Clava-herculis (“Prickly Ash”) {A Zanthoxylum Clava-herculis Preparation; (A
“Prickly Ash Preparation”); Dried Bark; Positive Dopaminergic/Noradrenalinergic/
Adrenalinergic [Active: Magnoflorine/Candicine/Lauriflorine/Nitidine
(Catechol-O-Methyltransferase Inhibitor)]; Transporter Activator [Active: Chelerythrine
(Na+-K+ ATPase Activator by Inhibiting PKC)]; Positive/Negative Glutamatergic [Active:
Chelerythrine (Alanine Aminotransferase/Glutamate Oxaloacetate Transaminase Inhibitor)];
Other Active: N-Acetylanonaine, Tembetarine, Cinnamamide, Herculin, Neoherculin}

| | |
|---|---|
| 800 | Secrets of the Tribe, LLC P.O. Box 5264 Denver, CO |
| | 80217 U.S.A. (“SOTT”) |

1) Topall, G. and Laborit, H. J Pharm Pharmacol 41(11): 789-791, 1989

TABLE 28
Neurotransmission Ion
Treatment Substance [Treatment Substance Details]

| | |
|---|---|
| Dose in | |
| mg (Notes) | Treatment Substance Vendor [Notes] |

Potassium Aspartate [99 mg Potassium/K+ as Potassium Aspartate; See Asp herein]

| | |
|---|---|
| 99 | Bluebonnet |

TABLE 29A
Transporter Activator
Treatment Substance [Treatment Substance Details]

| | |
|---|---|
| Dose in | |
| mg (Notes) | Treatment Substance Vendor [Notes] |

Peganum Harmala (“Harmala”) {A Peganum harmala Preparation; (A “Harmala Preparation”);
Extract 5:1; Transporter Activator/Inhibitor [Active: Harmine (Increases EAAT1/EAAT2
Amount), Active: Harmine, Harmaline, Harmalan, Norharmanium (Inhibits PMAT)]; Positive
Monoaminergic [Active: Harmine, Harmaline, Harmane, Norharman (Monoamine Oxidase A
Inhibitor)]; Positive Histaminergic [Active: Harmaline (Histamine N-Methyltransferase
Inhibitor)]; Positive Cholinergic [Active: Harmaline (Acetylcholinesterase Inhibitor)]}

| | |
|---|---|
| 250 | Amalth |

L-Pyroglutamic Acid (“Pyroglutamate”) [See Table 45A]

| | |
|---|---|
| 1,000 | BulkSupps [Powder Put into Capsule] |

TABLE 29B
Transporter Inhibitor
Treatment Substance [Treatment Substance Details]

| | |
|---|---|
| Dose in | |
| mg (Notes) | Treatment Substance Vendor [Notes] |

D-Aspartic Acid (“D-Asp”) [See Table 45A]

| | |
|---|---|
| 3,000 | Biotech Nutritions ® P.O. Box 865 Novi, MI |
| | 48376 U.S.A. (“BiotechN”) |

Arachidonic Acid {Transporter Inhibitor/Activator [EAAT1/EAAT2 Inhibited; Increased
Arachidonic Acid inside BBB cell increases P-GP Amount]; Positive GABAergic [Metabolized
into TRPV1-R Agonist]; Positive Cannabinoidergic [Precursor to Anandamide/
2-Arachidonoylglycerol]}

| | |
|---|---|
| 350 | Manufactured for Element Nutraceuticals 40 West |
| | 9 Mile Road, Suite 144 Pensacola, FL 32534 U.S.A. |
| | (“ElementN”) |

Taurine [See Table 45A]

| | |
|---|---|
| 500 | Now |

Threonine (“Thr”) [See Table 45A]

| | |
|---|---|
| 500 | Manufactured by Douglas Laboratories 600 Boyce Road |
| | Pittsburgh, PA 15205 U.S.A. (“Douglas”) |

Acetyl-L-Carnitine Hydrochloride (“Acetyl-Carnitine HCl”) [An Acetyl-Carnatine
Preparation] [See Table 45A]

| | |
|---|---|
| 500 | Jarrow |

Acetyl-Carnitine (“AC”), Alpha-Lipoic Acid (“AL”), Ubiquinone (“U”) [In One Capsule; See
Table 45A]

| | |
|---|---|
| 250 (AC) | Manufactured by DrVita Labs 6980 W. Warm Springs |
| 300 (AL) | Road Suite 100 Las Vegas |
| 200 (U) | NV 89113 U.S.A. (“NaturesLab”) |

Rauwolfia Serpentina (“Sarpagandha”) {A Rauwolfia serpentina Preparation; A
(“Sarpagandha Preparation”); Root Extract 5:1; Active: Reserpine; Negative Dopaminergic/
Serotoninergic/Adrenalinergic/Noradrenalinergic/Histaminergic [Active: Reserpine
(VMAT2/VMAT1 Inhibitor)]}

| | |
|---|---|
| 250 | Amalth |

Bee Propolis (“Propolis Extract”) [A Bee Propolis Preparation, A Propolis Preparation; 2:1
Extract; See Table 45A] [1]

| | |
|---|---|
| 500 | Y.S. ORGANIC BEE FARMS 2774 N. 4351 Rd. Sheridan, IL |
| | 60551 U.S.A. (“Y.S. Farms”) |

Rhus succedanea (“Fisetin”) {A Rhus succedanea Preparation; A (“Fisetin Preparation”); Stem;
Active: Fisetin; Transporter Inhibitor [BCRP/MCT1 Inhibitor]; Positive BDNFic [Increases
BDNF Amount]}

| | |
|---|---|
| 100 | SwansonHP |

Lobelia inflata (“Lobelia”) {A Lobelia inflata Preparation; A (“Lobeline Preparation”); Dried
Herb; Active: Lobeline; Positive Serotoninergic/Dopaminergic/Noradrenalinergic [Active:
Lobeline (SERT/DAT Inhibitor)]; Negative Histaminergic/Adrenalineergic/
Noradrenalinergic/Dopaminergic/Serotoninergic [Active: Lobeline (VMAT2 Inhibitor)];
Negative/Positive Cholinergic [Active: Lobeline (Agonist/Possible Antagonist for Nicotinic
ACh-R with Alpha4/Beta2/Alpha7 Subunit)]; Negative Opioidergic [Active: Lobeline (Mu-R
Antagonist)]}

| | |
|---|---|
| 800 | SOTT |

Loperamide Hydrochloride (“Loperamide”) {A Loperamide Preparation; Tablet Crushed to
Powder and Put into Capsule; Negative Glutamatergic [NMDA-R Inhibitor (e.g., Pore
Blocker/Dizocilpine Site Antagonist)]; Transporter Inhibitor [P-GP Inhibitor]}

| | |
|---|---|
| 2 | Active ingredient made in Italy, Distributed by |
| | Johnson & Johnson Consumer Inc. 7050 Camp Hill |
| | Rd. Fort Washington, PA 19034 U.S.A. (“J&J”) |

Pueraria Mirifica [“Pueraria mirifica Extract”] {A Pueraria Mirifica Preparation; 5000 mg
Equivalent as 500 mg 10:1 Extract; Active: Daidzin, Daidzein, Genistein, Deoxymiroestrol,
Genistin, Coumestrol; Positive/Negative GABAergic [Active: Genistein (GAT1 Inhibitor);
Active: Genistein, Daidzein (GABAA-R Antagonist); Active: Genistein (Decreases KCC2
Phosphorylation that Decreases KCC2 Activity); Active: Daidzein (Carbonic Anhydrase V/
VII Inhibitor)]; Negative Glutamatergic [Active: Genistein, Daidzein (NMDA-R Inhibitor)];
Negative Glycinergic [Active: Genistein, Daidzein (Gly-R NAM)]; Negative Seratoninergic/
Dopaminergic/Noradrenalinergic/Adrenalinergic/Catecholaminergic/Melatoninergic
[Active: Genistein (Aromatic L-Amino Acid Decarboxylase Inhibitor)]; Positive
Cannabinoidergic [Active: Genistein, Daidzein (Fatty Acid Amide Hydrolase Inhibitor)];
Transporter Inhibitor [Active: Genistein, Daidzein (P-GP/BCRP/MRP1 Inhibitor); Active:
Daidzin (MRP4/MRP5 Inhibitor)]; Positive BDNFic [Active: Genistein, Daidzein (Increases
BDNF Amount)]}

| | |
|---|---|
| 500 | SMS Health Products, 21049 Community St. Canoga |
| | Park, CA 91304 USA (“SMS”) |

Malus sylvestris/domestica (“98% Phloretin”) {A Malus sylvestris/domestica Preparation; A
(“Green Apple Preparation”); A Phloretin Preparation; Extract Powder; Active: 98% Phloretin;
Positive GABAergic [Active: Phloretin (GAT1/GAT2/GAT3/BGT1 Inhibitor)]; Transporter
Inhibitor [Active: Phloretin (P-GP/BCRP/MRP1/MCT1/MCT2/MCT4 Inhibitor)]}

| | |
|---|---|
| 10 | KAN |

Rheum palmatum (“Turkey Rhubarb”) {A Rheum genus Preparation, A Rheum palmatum
Preparation; A (“Rhubarb Preparation”); Root; See Table 46 }

| | |
|---|---|
| 500 | SwansonHP |

Sophora japonica (“Alpha-Glycosyl Isoquercitrin”) {A Sophora japonica Preparation; A
(“Alpha-Glycosyl Isoquercitrin Preparation”); Flower and Buds; Active: Alpha-Glycosyl
Isoquercitrin; Possible Transporter Inhibitor [Active: Alpha-Glycosyl Isoquercitrin (P-GP
Possible Inhibitor as Chemically Similar to Isoquercitrin)]; See Table 46B}

| | |
|---|---|
| 33 | Integrative Therapeutics, Inc. 825 Challenger Drive, |
| | Green Bay, WI 54311 USA (“Integrative”) |

Propionyl-L-Carnitine HCl (“Propionyl-Carnitine”) {A Propionyl-Carnitine Preparation;
Tablet; Transporter Inhibitor [OCTN2, ATB(0, +) Competitive Inhibitor]; Carnitine
Precursor/Prodrug}

| | |
|---|---|
| 333 | Manufactured for Cardiovascular Research, Ltd. 1061-B |
| | Shary Circle, Concord, CA 94518 USA (“CVRL”) |

Sodium Butyrate {Ratio of 1,200 mg Butyric Acid [Negative GABAergic (Butyric Acid
Inhibits VGAT)] and 313 mg Sodium/Na+ [Neurotransmission Ion (e.g., Glutamatergic Ion)]}

| | |
|---|---|
| 1,513 | Manufactured by BodyBio Inc 45 Reese Road, Millville, NJ |
| | 08332 U.S.A. (“Bodybio”) |

Phenylpiracetam {Positive Dopaminergic/Noradrenalinergic [NET/DAT Inhibitor (Stronger
Inhibitor for DAT)]; Positive Glutamatergic [Injection Increased NMDA-R Amount]; Positive
Cholinergic [Injection Increased Nicotinic ACh-R Amount]}

| | |
|---|---|
| 100 | NoopDepot [Powder Placed into Capsule] |

Uridine 5′-Monophosphate Disodium Salt (“Uridine”) {A Uridine Preparation; Transporter
Inhibitor [ENT1/ENT2 Inhibitor]}

| | |
|---|---|
| 300 | Manufactured For Double Wood LLC, 3510 Scotts Ln Ste 219, |
| | Philadelphia, PA 19129-1573 U.S.A. (“Double Wood”) |

Cinchona officinalis (“Cinchona”) [A Cinchona officinalis Preparation, A (“Cinchona
Preparation”); Bark; Active: Quinine/Quinidine, A (“Quinine/Quinidine Preparation”); See
Table 45A]

| | |
|---|---|
| 200 | Terra Vita Fine Whole Herbs, 51 Mountainash Rd. |
| | P.O. Box 15010, Brampton Ontario Canada. |
| | (“Terra Vita”) |

1) Braakhuis, A. Nutrients 2019 11(11): 2705

TABLE 30A
Adenosine Receptor Agonist/Adenosine Precursor/Adenosine Prodrug
Treatment Substance [Treatment Substance Details]

| | |
|---|---|
| Dose in | |
| mg (Notes) | Treatment Substance Vendor [Notes] |

Inosine [See Table 45A]

| | |
|---|---|
| 500 | SwansonHP |

Incarvillea sinensis {A Incarvillea sinensis Preparation; 40:1 Extract; 200 mg Tablet Contains
8000 mg of Incarvillea sinensis Fresh Herb Equivalent; Positive Adenosinergic [Active:
Incarvillateine (A1-R Agonist)]; Positive Opioidergic [Active: Incarvillateine (Mu-R/Kappa-R
Agonist)]}

| | |
|---|---|
| 200 | ABotanicals |

Methionine (“Met”) {Positive Adenosinergic [Precursor to SAMe (See 50% SAMe); SAMe
Precursor to Adenosine]; Negative Tyrosineic [Phenylalanine Hydroxylase Inhibitor]}

| | |
|---|---|
| 500 | SwansonHP |

Gastrodia elata (“Gastrodia”) {A Gastrodia elata Preparation; Tuber Extract; 970 mg per mL
Liquid; Transporter Activator [Active: N6-(4-hydroxybenzyl)adenine Riboside) (ENT1
Activator)]; Positive/Negative GABAergic [Active: Gastrodin (Reduces GABA
Transaminase/Succinic Semialdehyde Dehydrogenase Amount); Active: Gastrol, Gastrodin,
Bis(4-hydroxybenzyl)sulfide, N6-(4-hydroxybenzyl)adenine Riboside) (Possible GABAA-R
Agonist)]; Positive Adenosinergic [Active: N6-(4-hydroxybenzyl)adenine Riboside) (A2A-R
Activator)]; Positive Dopaminergic/Noradrenalinergic/Adrenalinergic [Active: Gastrol,
Gastrodin, Bis(4-hydroxybenzyl)sulfide, N6-(4-hydroxybenzyl)adenine Riboside (Possible
Aldehyde Dehydrogenase Inhibitor)]} [2, 3, 4, 5]

| | |
|---|---|
| 970 | HawaiiP |

1) Frenguelli, B. G. et al. J Neurochem. 2003 86(6): 1506-1515;
2) Hayashi, J. et al. Phytochemistry 2002 59(5): 513-519;
3) Ref. 235, Table 2;
4) Ref. 56, Table 6;
5) Huang, N. K. et al. J Nat Prod 2007 70(4): 571-574;
6) Ref. 118, Table 4

TABLE 30B
Adenosine Receptor Antagonist
Treatment Substance [Treatment Substance Details]

| | |
|---|---|
| Dose in | |
| mg (Notes) | Treatment Substance Vendor [Notes] |

Caffeine [Anhydrous Caffeine; A Caffeine Preparation; See Table 45A] [1]

| | |
|---|---|
| 200 | Nutrakey Health Performance, LLC 975 Bennett Drive |
| | Longwood, FL 32750 U.S.A. (“Nutrakey”) |

Theobroma cacao (“99% Theobromine”) [A Theobroma cacao Preparation; A (“Theobromine
Preparation”); Extract; Active: 99% Theobromine; See Table 45A] [2]

| | |
|---|---|
| 400 | Barlowe |

1) Pohanka, M. and Dobes, P. Int J Mol Sci 2013 14(5): 9873-9882;
2) Ref. 101, Table 4

TABLE 31
Ketogenic Treatment Substance
Treatment Substance [Treatment Substance Details]

| | |
|---|---|
| Dose in | |
| mg (Notes) | Treatment Substance Vendor [Notes] |

Medium Chain Fatty Acids (“55% Caprylic Acid and 35% Capric
Acid”) {A (“Caprylic Acid and Capric Acid Preparation”);
Active: 1652 mg Caprylic Acid (“C8”) and 1052 mg Capric Acid (“C10”)
of 3000 mg Medium Chain Fatty Acids from Palm Oil; A (“Palm Oil
Preparation”); A Elaeis guineenis/oleifera Preparation; Liquid Capsules;
Positive Ketogenic [Active: Caprylic Acid, Capric Acid (Precursor to Ketone
Bodies such as Acetoacetate/Beta-Hydroxybutyrate)]; Negative Glutamatergic
[Active: Caprylic Acid, Capric Acid (Precursor to Acetoacetate and Beta-
Hydroxybutyrate; Acetoacetate VGLUT1/VGLUT2/VGLUT3/VEAT Inhibitor; Beta-
Hydroxybutyrate VGLUT2 Inhibitor); Active: Capric Acid (AMPA-R Channel Blocker)];
Positive GABAergic/Negative Glutamatergic [Active: Caprylic Acid, Capric Acid
(Precursor to Acetoacetate/Beta-Hydroxybutyrate that are Precursors to ATP; ATP
promotes Opening of KATP Channel to Increase Membrane Hyperpolarization/Reduces
Excitatory Neurotransmission)]; Positive Glutamatergic/GABAergic [Active:
Caprylic Acid, Capric Acid (Precursor to Acetoacetate/Beta-Hydroxybutyrate that are
Precursors to Gln; Gln Precursor to Glu; Glu Precursor to GABA)]; Negative/Positive
Purinergic [Active: Caprylic Acid, Capric Acid (Precursor to Acetoacetate/Beta-
Hydroxybutyrate that Inhibit VNUT; Acetoacetate/Beta-Hydroxybutyrate Precursor ATP, ADP,
Adenosine)]; Positive Glutamatergic/Positive GABAergic/Negative Purinergic [Active:
Caprylic Acid (BCAA Precursor to Glu that is Precursor to GABA Or BCAA Used as Precursor
to ATP and ATP Precursor to ADP and Adenosine; Caprylic Acid Reduces BCAA Degradation
into ATP through BCKA Degradation by Branched Chain Alpha-Keto Acid Dehydrogenase
Complex by Inhibiting Branched Chain Alpha-Keto Acid Dehydrogenase Kinase that is
a Branched Chain Alpha-Keto Acid Dehydrogenase Complex Inhibitor)]; Transporter
Inhibitor [Caprylic Acid OAT3 Inhibitor]}

| | |
|---|---|
| 1652 mg | Manufactured For Nature's Nutrition |
| (C8) | 14725 S Porter Rockwell Blvd Ste G |
| 1052 mg | Buffdale, UT 84065 USA |
| (C10) | (“NatureNut”) |

Caprylic Acid (“60% Caprylic Acid”) [A (“Caprylic Acid Preparation”)
that may comprise Capric Acid; 600 mg from 1000 mg of Medium Chain Fatty Acids;
Softgel; See 55% Caprylic Acid and 35% Capric Acid Above]

| | |
|---|---|
| 1000 | Now |

Sodium Beta-Hydroxybutyrate (“83% Beta-Hydroxybutyrate”) {112.525 mg
Sodium/Na+ [Neurotransmission Ion (e.g., Glutamatergic Ion)] and 530.475 mg
Beta-Hydroxybutyrate, A (“Beta-Hydroxybutyrate Preparation”); See 55% Caprylic
Acid and 35% Capric Acid Above}

| | |
|---|---|
| 643 | BulkSupps [Powder placed in |
| | Capsule] |

D-Beta-Hydroxybutyrate-D-1,3-Butanediol (“D-Beta-Hydroxybutyrate Ester,” “DBHE”)
Beta-Hydroxybutyrate Prodrug with Improved Bioavailability; A (“Beta-Hydroxybutyrate
Ester Preparation”); A (“Beta-Hydroxybutyrate Preparation”); See Sodium
Beta-Hydroxybutyrate Above; and Potassium (“K”) [Neurotransmission Ion];
Both in Same Liquid in Container; See Table 46B}

| | |
|---|---|
| 30,000 | KetoneAid Inc, 6304 Crosswoods Circle, |
| (DBHE) | Fall Church, VA 22044, USA |
| 99 | (“KetoneAid”) |
| (K) | |

TABLE 32
Enzyme Cofactor
Treatment Substance [Treatment Substance Details]

| | |
|---|---|
| Dose in | |
| mg (Notes) | Treatment Substance Vendor [Notes] |

Alpha-Lipoic Acid [Racemic; An (“Alpha-Lipoic Acid Preparation”); See Table 45A]

| | |
|---|---|
| 50 | SwansonHP |
| 100 | WFoods |

Sodium R-Fraction Alpha-Lipoic Acid (“R-Alpha-Lipoic Acid”) [An (“Alpha Lipoic Acid
Preparation”); See Table 45A] [1]

| | |
|---|---|
| 50 | SwansonHP |

S-Adenosyl L-Methionine Tosylate Disulfate (“50% SAMe”) {Active: 400 mg SAMe From
800 mg SAMe Tosylate Disulfate; A (“SAMe Preparation”); Tablet; Negative Dopaminergic/
Noradrenalinergic/Adrenalinergic/Histaminergic/Putrescinergic/Spermidinergic [Active:
SAMe (Cosubstrate/Cosubstrate Precursor that Promotes Neurotransmitter Degradation)];
Positive Adrenalinergic/Spermidinergic/Sperminergic/Melatoninergic [Active: SAMe
(Cosubstrate/Cosubstrate Precursor that Promotes Neurotransmitter Production)]; Positive
Purinergic [Precursor to Adenosine]}

| | |
|---|---|
| 400 | SwansonHP |

Manganese Bisglycinate {A (“Manganese Preparation”); Positive Glutamatergic/GABAergic
[Active: Manganese (Glutamine Synthetase Cofactor)]}

| | |
|---|---|
| 15 | 29483 USA (“Thorne”) |
| | Thorne Research, Inc. Industrial Road, 620 Onmi |
| | Dr., Summerville, SC |

Ascorbyl Palmitate and Dibasic Calcium Phosphate (“Ascorbyl Palmitate, Calcium
Phosphate”) [203 mg Ascorbyl Palmitate (Ascorbyl Metabolized into Ascorbic Acid;
Antioxidant, Cofactor for Various Enzymes) and 102 mg Dibasic Calcium (See Table 45A)
Phosphate Per 500 mg Tablet]

| | |
|---|---|
| 500 | SNaturals |

Ascorbic Acid (“VC”) and Sunflower Oil and Lecithin (“Liposomal Formulation,” “LF”) [VC
(Antioxidant, Cofactor for Various Enzymes) and LF in the Same Softgel]

| | |
|---|---|
| 333 (VC) | Manufactured for and Distributed by: IV for Life, |
| | 2973 Harbor Blvd. |
| 117 (LF) | #322 Costa Mesa, CA 92626 (“IV for Life”) |

1) Salehi, B. et al. Biomolecules 2019 9(8): 356

TABLE 33A
Adrenaline/Noradrenaline Receptor Inhibitor
Treatment Substance [Treatment Substance Details]

| | |
|---|---|
| Dose in | |
| mg (Notes) | Treatment Substance Vendor [Notes] |

Corynanthe johimbe/Pausinystalia yohimbe (“Yohimbe Bark”) {A (“Corynanthe
johimbe/Pausinystalia yohimbe Preparation”); Bark; Active: Yohimbine (Rauwolscine and
Corynanthine Mixture); A (“Rauwolscine/Corynanthine Preparation”); A (“Yohimbine
Preparation”); See Table 45A]

| | |
|---|---|
| 600 | SwansonF1 |

Rauwolfia vomitoria (“99% Rauwolscine”) [A Rauwolfia vomitoria Preparation; Root
Extract; Active: 99% Rauwolscine (“Alpha-Yohimbine”); A (“Rauwolscine Preparation”); See
Yohimbe Bark Above (Activity Similar But Rauwolscine a Strong Alpha2-R Antagonist
Relative to Corynanthine)]

| | |
|---|---|
| 3 | Manufactured for Gorilla Mind, LLC, 7154 W State |
| | Street, STE 138, Boise, ID 83714 USA |
| | (“GorillaM”) |

TABLE 33B
Additional Treatment Substance
Treatment Substance [Treatment Substance Details]

| Dose in | |
|---|---|
| mg (Notes) | Treatment Substance Vendor [Notes] |
| | Inositol [See Table 45A] [1, 2, 3, 4] |
| 500 | Now |

Mucin (“Mucin/Sialac Acid Preparation”) [A Mucin Preparation; Extract; Active: Sialic Acid
(Neuron Biochemical Component); A (“Sialac Acid Preparation”)] [5]

| | |
|---|---|
| 500 | Ecological Formulas 1061-B Shary Circle Concord, CA |
| | 94518 (“EcoFormulas”) |

Garcinia cambogia (“50% Hydroxycitric Acid”) {A Garcinia cambogia Preparation, Fruit
Extract; Active: 250 mg Hydroxycitric Acid Per 500 mg; A (“Hydroxycitric Acid Preparation”);
Possible Positive Glutamatergic/GABAergic [Active: Hydroxycitric Acid (Inhibits ATP Citrate
Lyase Oxaloacetate Production) (See Oxaloacetate Above)]}

| | |
|---|---|
| 500 | Solgar, Inc. 500 Willow Tree Road, Leonia NJ 07605 |
| | USA (“Solgar”) |

1) Fisher, S. K. et al. J. Neurochem. 2002 82(4): 736-754;
2) Gamper, N. and Shapiro, M. S. Nat Rev Neurosci. 2007 8(12): 921-934;
3) Levine, J. et al. Am J Psychiatry. 1995 152(5): 792-794;
4) Fux, M. et al. Am J Psychiatry. 1996 153(9): 1219-1221;
5) Wang, B. and Brand-Miller, J. Eur J Clin Nutr 2003 57(11): 1351-1369;
6) Ref. 9, Table 17A

### Example 2: Measurements of TSF and Other Sexual Responses

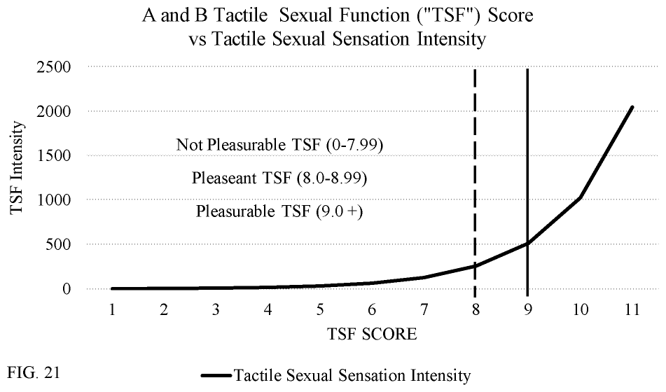

FIG. 21 depicts the relative intensity of TSF for the A score and B score. For a male, the A score is the highest intensity of TSF just before the first muscle contraction of ejaculation, and the B score is the highest intensity of TSF just after the first muscle contraction of ejaculation. The intensity of TSF shown at the vertical axis is about twice as strong for each unit of score increase. For example, a 2 score is about twice as intense in sexual sensation as a 1.0 score; a 9.5 score is about twice as intense in sexual sensation as an 8.5 score. This scale sexual sensation intensity describes the large range of differences between a barely detectable by sensory perception TSF score of 1.0, the non-pleasurable sexual sensation of TSF middle-range scores such as 4.0, 5.0, etc., and a strong, pleasurable, almost full recovery TSF of 10.0. For example, the A scores of 1.0, 2.0, 3.0, 4.0, 5.0, and 10.0 have approximate sexual sensation intensities of about 2, 4, 8, 16, 32, and 1024 respectively. An A and/or B score of 0.0 is no TSF (no sexual sensation), while a score above 0.0 and below 9.0 is a sexual sensation, but the sensation is not pleasurable at a score above 0.0 and below 8.0, or weakly pleasurable (“pleasant”) at a score of from 8.0 to below 9.0. An A/B score from 9.0 to below 10.0 is sexual pleasure, but weak relative to normal TSF before SRSS began. To provide a consistant description of low, middle, and high TSF scores in a subrange (e.g., 9.0 to 9.99), decimal scores of 0.2, 0.5 and 0.8 added to the integer of the subrange respectively (e.g., 9.2, 9.5, 9.8). An A/B score from 10.0 to below 11.0 is in the normal range sexual intensity before the beginning of SRSS (e.g., a 10.2 score is a low but normal TSF A/B score, a 10.8 score indicates a high but normal TSF A/B score). An A/B score from 11.0 and above are TSF above normal range in sexual pleasure intensity, wherein the intensity of TSF was greater than occurred before the beginning of SRSS.

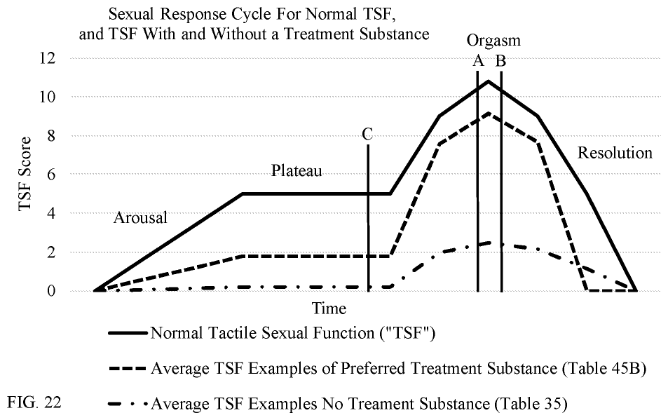

FIG. 22 depicts normal TSF during the sexual response cycle and TSF after the beginning of SRSS with and without ingestion of a treatment substance. The C score is the highest level of TSF during the plateau phase on a 0.0 to 5.0 scale, with a score of 0.0 being no detectable TSF, 1.0 being a weakly pleasant (“poor”) TSF, 2.0 being a pleasant (“fair”) TSF, 3.0 being a below normal but sexual pleasure (“good”) TSF, 4.0 being a below normal TSF before SRSS began but strong sexual pleasure (“very good”) TSF, and 5.0 being a normal, full recovery sexual pleasure (“excellent”) TSF, and any C score TSF above 5.0 being greater than occurred before the beginning of SRSS. The sensory perception of the C score was generally a linear increase in sexual sensation intensity for each point of TSF score increase, rather than a doubling of intensity for each point of increase in TSF as described for the A and B scores. The C score TSF sensation was typically localized to the skin, however the A and B scores were a more generalized TSF sensation throughout the penis. FIG. 22 depicts the average C, B, and A scores for TSF for all examples from Table 35 wherein no treatment substances were ingested; and for Table 45B the improvement in TSF where preferred treatment substances were ingested and a preferred A/B score of at least 8.0 in combination with a preferred C score of at least 2.0 and/or a more preferred A/B score of at least 9.0 regardless of the C score were recorded. The average of both the A and B scores from Tables 35 and Table 45B were used as the peaks depicted during the orgasm phase for the line graphs for those Tables.

Variations in TSF and other sexual responses were noticed after ingesting a treatment substance, and additional scores to describe these responses were recorded in some examples. In some examples the C score for TSF was localized only to the head (“glands”) of the penis when one or more C score measurements were taken prior to attempted or achieved ejaculation/orgasm. When all C score(s) for an example were located only to the head of the penis this sexual response was a “CT” score of “Yes,” otherwise the CT score was “No.” For example, when final C score measured was 0 (no sexual sensation) but an earlier C score measurement in the same example had sexual sensation, then the CT score was scored “Yes” or “No” as appropriate (e.g., Examples T41:4, T41:2). When all C score(s) for an example were 0 the CT score was scored “N/A.” After the beginning of SRSS, the time duration of the A score and B score when some (e.g., weak, subnormal) TSF occurred without ingestion of a treatment substance was the same as the normal sexual responses before the beginning of SRSS. Often after ingesting a treatment substance the time duration of the B score (and occasionally the A score) was shorter than during the normal TSF before the beginning of SRSS. In some examples after ingesting a treatment substance, the speed of B score decrease from the first muscle contraction to the last muscle contraction of ejaculation was recorded as the “BS” score using terms such as “IS” for instantaneous loss of TSF where a B score of 0.0 occurred at or immediately after the first muscle contraction, “FT” for a fast loss of TSF, and “SL” for a slow loss of TSF. In some examples the minimum B score measured at or near the last muscle contraction was recorded as the “BM” score using the same scoring parameters as for the B score. In some examples the strength of the muscle contractions during ejaculation were recorded as the “D” score, with a score of 1.0 being very weak muscle contractions, 2.0 being weak muscle contractions, 3.0 being typical/normal strength muscle contractions, 4.0 strong muscle contractions, and 5.0 being very strong muscle contractions. In some examples the ease to achieve ejaculation was recorded as the “DE” score, with a score of 1.0 being ejaculation achieved with some difficulty, 2.0 being a typical/normal ease of ejaculation, and a 3.0 being of the ease of ejaculation was promoted. When failure to ejaculate occurred (i.e., no muscle contractions) “FE” is listed for the B, D, and DE scores, and the B, D and DE numeric scores were 0.0 for mathematical evaluations (e.g., statistical analysis) in such occurrences. When an erection was achieved, but TSF was unable to go from the plateau phase to orgasm (no A and B scores) and failure to ejaculate occurred, then “FO” for the failure to orgasm is listed for the A, B, D, and DE scores, and the A, B, D and DE numeric scores were 0.0 for mathematical evaluations in such occurrences. When a score (e.g., C, CT, A, B, BM, BS, D, DE) was not recorded or not applicable then “N/A” is listed for that score and that score was not used (no numeric score) for mathematical evaluations in such an occurrence. It is contemplated that a DE score greater than 2.0 may reverse the inability to ejaculate/orgasm, as well as promote the ability ejaculate/orgasm, during sexual stimulation with a partner during sexual intercourse (e.g., even promote premature ejaculation, though regaining the ability to naturally reproduce is a preferred quality of life improvement). It is contemplated that scores for sexual responses (e.g., A, B, BS, BM, C, D, DE, etc) described herein are applicable for measuring the sexual responses of a woman and/or may be readily modified to be applicable for measuring sexual responses of a woman. For example, the C score may be used to score a woman's TSF during a plateau phase; and the A and B scores for TSF are may used to score a woman's TSF just before and right after orgasm begins, respectively.

Table 34 shows the A/B scores of TSF described in examples of quality-of-life terms. Scores of 8.8-8.99 are just under a crossover point where TSF is recognizable sexual pleasure intensity relative to TSF before SRSS began, and scores of 10.8-10.99 was the top of the range of sexual pleasure before SRSS began.

TABLE 34
Examples of Quality-of-Life for A and/or B Scores

| A/B Score | Quality of Life Conversion |
|---|---|
| 0.0 to 7.99 | Not a pleasurable sexual sensation (if any sexual sensation) that is of |
| | scientific interest only. In a movie, Hell was shown with various torments |
| | being used. One soul was tormented for all eternity by being in a room full |
| | of attractive and welcoming women, but upon each step of approaching |
| | them, they would fade away and finally disappear when he reached them. It |
| | is contemplated that the living, men and women, having SRSS or other |
| | sexual dysfunctions, fully understand this damnation. No change in a |
| | voluntary celibate lifestyle (See Example 3). |
| 8.0-8.99 | Weakly pleasurable (“Pleasant”). |
| 8.8 | Not enough to start dating, but enough to continue a happy preexisting |
| | relationship. |
| 8.5 | In a preexisting relationship: Is her personality unicorn level? If the answer |
| | is “No” see the 8.2 A/B quality of life conversion below. |
| 8.2 | Adios babe! I am going to work on my fishing lure designs! |
| 9.0-9.99 | Enough to start dating again. Worth the cost of lunch and a matinee movie |
| | on a date. |
| 10.0-10.99 | Worth the cost of dinner and a full price movie on a date. |
| 11.0 and above | I'm going to Vegas! |

Before engaging in ingesting treatment substances of the present invention most instances of A, B, and C scores were measured as 0.0. Some instances of TSF of varying levels above 0.0 scores for A, B, and C occurred, and but for rare exceptions the A and B scores were 8.8 or less, and 1.0 or less scores (e.g., 0.0) for the C score. It is contemplated that natural variations in the concentration of one or more neurotransmitters/neurotransmitter receptors, physiological condition (e.g., insufficient sleep tends to associated with increased TSF) and/or the effects of one or more substances in the diet (e.g., a nutritional supplement, food) that act as treatment substance(s) may promote instances of mild sexual function(s) or even increase the effect of SRSS (e.g., a treatment substance in the diet that reduces a sexual function). For example, caffeine has an average plasma half-life of 5 hours [Institute of Medicine (U.S.) Committee on Military Nutrition Research. Caffeine for the Sustainment of Mental Task Performance: Formulation for Military Operations. Washington (DC): Nation Academies Press (U.S.); 2001] and is a treatment substance that promotes sexual functions as described herein; and it is contemplated that any noticeable effect of caffeine as a treatment substance on sexual function may last for one or more half-lives. It would be preferred to avoid the residual influence of a previously ingested treatment substance by allowing at least 5 average biological half-lives for that treatment substance to occur before either measuring sexual functions without the influence of a treatment substance, or ingesting another treatment substance to measure that treatment substance's effect on sexual functions. But to rapidly obtain measurements of sexual functions described herein under conditions similar to normal daily life, unless otherwise noted, the following examples of measurements of sexual functions for both no treatment substance ingested or a treatment substance ingested occurred 6 hours or more after ingesting a previous treatment substance described herein, food, a nutritional supplement, and/or a beverage containing caffeine or alcohol. Beverages ingested with a treatment substance are noted herein as it is contemplated that they may contribute to a sexual function and/or side effect particularly when they contain the treatment substances caffeine or alcohol. Noted herein, melatonin in one or more doses between about 10 PM the previous day until about 4 AM the day of measurements prior to and during periods of insomnia that normally occur daily, wherein the inventor typically would wake up one or more periods of time that added up to about 1.5 to 2.5 hours total each night, and ingesting a dose of a part of a melatonin tablet each period of wakefulness to promote going back to sleep. About 0.3 mg to about 1.0 mg from part of tablet was ingested with water for each dose (vendors: Wall-Mart Stores Inc. 702 S.W. 8^{th} St. Bentonville, AK, 72716; SNaturals with about 0.1 mg to about 0.2 mg of pyridoxine HCl and about 2.8 mg to about 5.6 mg of calcium as dibasic calcium phosphate in each dose of melatonin; and CVS Pharmacy Inc. One CVS Drive, Woonsocket RI, 02895, with about 1 mg to about 2.0 mg of pyridoxine HCl and about 8 mg to about 16 mg of calcium as dibasic calcium phosphate in each dose of melatonin).

Table 35 shows working examples of measurements of the inventor's TSF (e.g., A, B, BM, and C scores) and other aspects of sexual function (e.g., BS, D, DE scores) wherein no treatment substance was ingested for evaluation. The measurements of Example 35:1 is also recorded in Example 4 where no physical and psychological sexual stimulation occurred for at least 21 days before taking measurements of sexual functions. No food, dietary vitamins or nutritional supplements were ingested the day of the measurements until after the measurements were taken, unless otherwise noted herein. For examples T35:2, T35:3, T35:17, T35:21, T35:27, T35:28, and T35:39 measurements were conducted after a full night's sleep (enough sleep to be fully rested despite sleep interruptions from insomnia) and no noticeable effects of alcohol consumption the previous evening, if alcohol was ingested; except for example T35:17 the effects of excess alcohol consumption from the night before (e.g., reduced cognitive function and fatigue) were present. Coffee, vitamins and NAC was likely ingested more than 5 hours prior to example T35:46. Measurements occurred between in the morning from about 2:30 AM to about 11:00 AM, except for example T35:46 which occurred at about 6:00 PM and example T35:1 where the time of day was not recorded.

TABLE 35
Sexual Function Scores With No Treatment Substance Ingested

| Example | C | CT | A | B | BM | BS | D | DE |
|---|---|---|---|---|---|---|---|---|
| T35: 1 | 0.0 | N/A | 0.0 | 0.0 | 0.0 | N/A | 3.0 | 2.0 |
| T35: 2 | 0.0 | N/A | 0.0 | 0.0 | 0.0 | N/A | 3.0 | 2.0 |
| T35: 3 | 0.0 | N/A | 0.0 | 0.0 | 0.0 | N/A | 3.0 | 2.0 |
| T35: 4 | 0.0 | N/A | 0.0 | 0.0 | 0.0 | N/A | 3.0 | 2.0 |
| T35: 5 | 0.0 | N/A | 0.0 | 0.0 | 0.0 | N/A | 3.0 | 2.0 |
| T35: 6 | 0.0 | N/A | 0.0 | 0.0 | 0.0 | N/A | 3.0 | 2.0 |
| T35: 7 | 0.0 | N/A | 0.0 | 0.0 | 0.0 | N/A | 3.0 | 2.0 |
| T35: 8 | 0.0 | N/A | 0.0 | 0.0 | 0.0 | N/A | 3.0 | 2.0 |
| T35: 9 | 0.0 | N/A | 0.0 | 0.0 | 0.0 | N/A | 3.0 | 2.0 |
| T35: 10 | 0.0 | N/A | 0.0 | 0.0 | 0.0 | N/A | 3.0 | 2.0 |
| T35: 11 | 0.0 | N/A | 0.0 | 0.0 | 0.0 | N/A | 3.0 | 2.0 |
| T35: 12 | 0.0 | N/A | 0.0 | 0.0 | 0.0 | N/A | 3.0 | 2.0 |
| T35: 13 | 0.0 | N/A | 0.0 | 0.0 | 0.0 | N/A | 3.0 | 2.0 |
| T35: 14 | 0.0 | N/A | 0.0 | 0.0 | 0.0 | N/A | 3.0 | 2.0 |
| T35: 15 | 0.2 | No | 0.0 | 0.0 | 0.0 | N/A | 3.0 | 2.0 |
| T35: 16 | 0.2 | No | 0.0 | 0.0 | 0.0 | N/A | 3.0 | 2.0 |
| T35: 17 | 0.2 | No | 0.0 | 0.0 | 0.0 | N/A | 3.0 | 2.0 |
| T35: 18 | 0.0 | N/A | 0.0 | 1.5 | N/A | N/A | 3.0 | 2.0 |
| T35: 19 | 0.0 | N/A | 0.0 | 1.5 | N/A | N/A | 3.0 | 2.0 |
| T35: 20 | 0.0 | N/A | 0.0 | 2.5 | N/A | N/A | 3.0 | 2.0 |
| T35: 21 | 0.0 | N/A | 0.0 | 2.5 | N/A | N/A | 3.0 | 2.0 |
| T35: 22 | 0.0 | N/A | 0.0 | 3.5 | N/A | N/A | 3.0 | 2.0 |
| T35: 23 | 0.0 | N/A | 0.0 | 3.5 | N/A | N/A | 3.0 | 2.0 |
| T35: 24 | 0.8 | No | 0.0 | 1.5 | N/A | N/A | 3.0 | 2.0 |
| T35: 25 | 0.0 | N/A | 1.5 | 2.5 | N/A | N/A | 3.0 | 2.0 |
| T35: 26 | 0.8 | No | 1.5 | 1.5 | N/A | N/A | 3.0 | 2.0 |
| T35: 27 | 0.0 | N/A | 2.5 | 2.5 | 0.0 | N/A | 3.0 | 2.0 |
| T35: 28 | 0.0 | N/A | 2.5 | 2.5 | N/A | N/A | 3.0 | 2.0 |
| T35: 29 | 0.0 | N/A | 2.5 | 2.5 | N/A | N/A | 2.5 | 2.0 |
| T35: 30 | 0.8 | No | 2.5 | 2.5 | N/A | N/A | 3.0 | 2.0 |
| T35: 31 | 0.0 | N/A | 2.5 | 3.5 | N/A | N/A | 3.0 | 2.0 |
| T35: 32 | 0.0 | N/A | 2.5 | 3.5 | N/A | N/A | 3.0 | 2.0 |
| T35: 33 | 0.8 | No | 2.5 | 2.5 | N/A | N/A | 3.0 | 2.0 |
| T35: 34 | 0.0 | N/A | 2.5 | 3.5 | N/A | N/A | 1.0 | 2.0 |
| T35: 35 | 0.0 | N/A | 3.5 | 3.5 | 3.5 | N/A | 3.0 | 2.0 |
| T35: 36 | 0.8 | No | 3.5 | 3.5 | N/A | N/A | 3.0 | 2.0 |
| T35: 37 | 0.0 | N/A | 3.5 | 3.5 | N/A | N/A | 3.0 | 2.0 |
| T35: 38 | 0.0 | N/A | 3.5 | 3.5 | N/A | N/A | 2.0 | 2.0 |
| T35: 39 | 0.2 | Yes | 3.5 | 3.5 | 3.5 | N/A | 3.0 | 2.0 |
| T35: 40 | 0.0 | N/A | 4.5 | 1.5 | N/A | N/A | 3.0 | 1.0 |
| T35: 41 | 0.0 | N/A | 4.5 | 4.5 | 4.5 | N/A | 3.0 | 2.0 |
| T35: 42 | 0.0 | N/A | 4.5 | 4.5 | 3.5 | N/A | 3.0 | 2.0 |
| T35: 43 | 0.8 | No | 5.5 | 5.5 | N/A | N/A | 3.0 | 2.0 |
| T35: 44 | 0.8 | No | 5.5 | 5.5 | N/A | N/A | 3.0 | 2.0 |
| T35: 45 | 0.8 | No | 5.5 | 5.5 | N/A | N/A | 3.0 | 2.0 |
| T35: 46 | 0.8 | No | 5.5 | 5.5 | N/A | N/A | 3.0 | 2.0 |
| T35: 47 | 0.8 | No | 7.5 | 7.5 | 0.0 | N/A | 3.0 | 2.0 |
| T35: 48 | 0.0 | N/A | 7.5 | 7.5 | N/A | N/A | 3.0 | 2.0 |
| T35: 49 | 0.8 | No | 7.5 | 2.5 | N/A | N/A | 3.0 | 2.0 |
| T35: 50 | 0.0 | N/A | 7.5 | 6.5 | 5.5 | N/A | 3.0 | 2.0 |
| T35: 51 | 0.8 | No | 8.5 | 8.5 | N/A | N/A | 3.0 | 2.0 |
| T35: 52 | 0.0 | N/A | 8.8 | 8.8 | 8.8 | N/A | 3.0 | 2.0 |

### Example 3: Women

The inventor is a male heterosexual. No sexual stimulation with a female partner occurred before developing SRSS. Sexual intercourse with female partners was conducted using a prophylactic (i.e., a condom) approximately 30 times in the years after developing SRSS. Full psychological focus on positive, erotic thoughts was needed to maintain an erection during sexual intercourse (i.e., the opposite of thinking about baseball). The TSF during each occurrence of sexual intercourse with a female partner was the same: A, B, C, D, and DE scores of 0 [i.e., no sexual sensation, and no ejaculation/orgasm (“FO” scores for D and DE, numeric scores of 0]. Direct tactile sexual stimulation (no condom) other than sexual intercourse by a female partner occurred approximately 10 times, with C scores of about 3 to about 4 occurring about 3 times, with no ejaculation/orgasm (i.e., A, B; D and DE numeric scores of 0); and the other 6 to 7 instances of direct tactile sexual stimulation by a female partner had A, B, C, D, and DE numeric scores of 0 (i.e., no sexual sensation, and no ejaculation/orgasm).

### Example 4: Monk Mode

Several years before any evaluations of the treatment substances described herein, the effect of a prolonged period of no physical and psychological sexual stimulation was evaluated. The inventor focused on work related matters in a home office, minimalized leaving the dwelling to avoid contact with women, avoided looking at any images or sound recording of people to avoid any images or recordings of women, and did not engage in any physical sexual stimulation for between 21 to 22 days. The next day, TSF for A, B, BM, and C scores were measured each as 0 (absence of sexual sensation), but ejaculation occurred with the D score was 3.0 (normal) and the DE score was 2.0 (normal).

### Example 5: D-Asp as a Possible Testosterone Booster

D-Asp increased testosterone production in rodent studies [Nagata, Y. et al. FEBS Lett 1999 444(2-3):160-164; Roshanzamir, F. and Safavi, S. M. Int J Reprod Biomed 2017 15(1):1-10]. D-Asp (Vendor: BiotechN) was ingested at 3000 mg/day. After about a week physical arousal (erections) were extremely promoted (with concern of being above normal, healthy range prior to SRSS), and a small bump from a varicose vein developed on the penis. TSF for A, B, BM, and C scores were measured each as 0, the D score was 3.0, and the DE score was 2.0. Daily ingestion of D-Asp was stopped.

### Example 6: Bupropion (“Wellbutrin”): Another Prescription Pharmaceutical

Upon consultation with a medical physician regarding the varicose vein of Example 5 and the loss of TSF, the inventor was referred to a urologist that prescribed bupropion to treat the loss of TSF. Bupropion inhibits dopamine and noradrenaline movement into cells and inhibits acetylcholine nicotinic receptors [Costa, R. et al. Drug Metab Rev. 2019 51(3):293-313]. Bupropion was ingested daily per the prescription's instructions for at least 3 months. During the first week of ingesting bupropion the side effect of feeling odd/under the influence of a drug occurred (i.e., felt not normal) but that side effect ended after the first week. After the first day of ingesting bupropion TSF for A and B scores were measured at about 4 each, the C score was either 0 or a low score (e.g., less than 1), the D score was 3.0, and the DE score was 2.0. Several additional measurements (at least two) of TSF occurred during the following months (at least 2 months), but in all instances the A, B, BM, and C scores were measured each as 0, the D score was 3.0, and the DE score was 2.0. Bupropion ingestion was stopped.

### Example 7: Cheers (Part 1)

Vitamins, nutritional supplements and food normally ingested by the inventor as part of the regular diet were ingested after a treatment substance evaluation to avoid adding these dietary substances as additional treatment substance variables to the results. Initial evaluations of different treatment substances (alone and in various combinations) listed in Example 1 and evaluations of differing periods to time of between ingestion the treatment substances (e.g., minutes up to over 20 hours) to A, B and C score measurements resulted scores below 9.0 for A and B measurements. Inspired by an episode of a comedy television show where a bartender successfully created a delicious new drink after many attempts, but failed to write down the ingredients, the variable of chance was introduced into some treatment substance evaluations. On some mornings the inventor closed his eyes and selected various doses of treatment substances by ingesting 1 to about 7 capsules/tablets from various bottles of previously evaluated treatment substances that were touched, wherein each bottle contained one treatment substance, all while keeping his eyes closed. The selection with eyes closed was semi-random as a subset of treatment substances that had the strongest positive effect on TSF were placed in hand's reach, and this treatment substance selection typically included bottles containing one or more BCAAs. On some, but not all, of these treatment substance evaluations the normal dietary vitamins and nutritional supplements ingested daily, using normal dosages by selection with eyes open, were also ingested just before or with the treatment substances. Coffee or water was used to ingest the treatment substances with or without dietary vitamins and nutritional supplements. An improvement in the measurement of TSF was noticed during some of these evaluations, and the nutritional supplement N-Acetyl Cysteine (“NAC”; typically, a 600 mg sustained and quick release tablet from Jarrow used during this evaluation period) was identified as a treatment substance. As NAC was ingested several times during the day as part of regular diet for years before the evaluation of treatment substances described herein, and no positive effect was noticed on TSF during those years, NAC and other dietary supplements were initially excluded from evaluations of treatment substances as being contemplated as having a neutral to possible negative effect on TSF based on these observations, and this was an unexpected result. After identification of NAC as a treatment substance, the positive effect of NAC was particularly noticed when combined with other treatment substance(s) (e.g., a positive GABAergic treatment substance) as described herein.

### Example 8: Cheers (Part 2) The Hangover, Full Recovery and More

In light of the success in improving TSF described in Example 7, the variable of chance was continued by ingesting a semi-random combination of treatment substances on several evaluations as described in Example 7. These evaluations occurred in the morning, typically when the inventor was having impaired cognitive function from ingesting an ample amount of alcoholic beverages the previous night. The inventor again placed a sub-set of treatment substance containers of capsules/tablets including some the most effective treatment substances, such as positive GABAergic activator treatment substances (e.g., one or more BCAAs such as Val, NGABA), NAC, and other treatment substances in front of the inventor, then with eyes closed the inventor reached out, picked up containers that were touched, and ingested multiple capsules/tablets. On one occasion of ingesting treatment substances the inventor was cognitively impaired by a combination of a lack of sleep (“sleep deprivation”) and the previous night's alcohol intoxication, a C score of 5 and enhanced A and B scores of above 11.0 (above normal range before SRSS) with normal responses of excitement, plateau, orgasm, and resolution (i.e., no rapid loss of sensation during the B score measurement during the resolution phase) were measured about 60 minutes after ingestion of treatment substances with coffee. Of note, though focus on positive erotic thoughts helps to maintain an erection, after ingesting a treatment substance no noticeable improvement in TSF occurred due to the positive erotic thoughts (i.e., all improvement in TSF was associated with the effect of the treatment substance), except for during this occasion wherein positive erotic thoughts helped promote TSF. In the years before treatment substances evaluation, the inventor noticed that days of A and B scores above 0 for TSF tended to occur while suffering from sleep deprivation of one night. Various neurotransmitter systems are affected both by lack of sleep (See Table 14) and alcohol consumption. It is contemplated that upon ingesting a treatment substance one or more scores of sexual function described herein may be increased above normal range, particularly for individuals (e.g., men, women) lacking a sexual dysfunction. For example, an A or B score of 11.0 and above and/or a C score above 5.0 are contemplated to occur upon ingesting a treatment substance described herein more often in a person who does not have a sexual dysfunction relative to person who does have a sexual dysfunction such as SRSS.

### Example 9: Improvement of TSF by Treatment Substances

Table 36A shows working examples of measurements of the TSF for the A, B, and C scores after ingestion of one or more treatment substances, wherein the treatment substances were ingested 60 minutes with water before the measurements. Table 36B shows the same working examples with additional sexual function measurements listed. Dietary vitamins and nutritional supplements were ingested about 7:00 AM to about 10:30 AM with coffee, and coffee consumption ended by about 12:00 PM or earlier. No food was ingested during the day before measurements as only one meal a day is typically eaten in the evening. All measurements were made in the afternoon from about 4:45 PM to about 5:45 PM, with few exceptions in the early evening such as T36:165 which was measured about 6:42 PM.

TABLE 36A
Treatment Substance Ingested with Water in the Afternoon

| Example | Treatment Substance [Details] | Vendor | mg | C | A | B |
|---|---|---|---|---|---|---|
| T36: 1 | MagT | Now/PP/SN | 667 | 3.5 | 8.5 | 8.8 |
| | NAC.SR | Jarrow | 600 | | | |
| | Gly | Now | 1000 | | | |
| T36: 2 | MagT | Now/PP/SN | 2000 | 1.5 | 5.8 | 8.8 |
| T36: 3 | MagT | Now/PP/SN | 667 | 1.5 | 5.5 | 8.5 |
| | Taurine | Now | 500 | | | |
| T36: 4 | MagT | Now/PP/SN | 667 | 1.5 | 3.5 | 7.5 |
| | Acetyl-Carnitine HCl | Jarrow | 500 | | | |
| T36: 5 | MagT | Now/PP/SN | 667 | 1.0 | 4.5 | 3.5 |
| | 5HTP | Bluebonnet | 100 | | | |
| T36: 6 | MagT | Now/PP/SN | 667 | 1.5 | 4.5 | 7.5 |
| | AKG | Klaire Labs | 300 | | | |
| T36: 7 | NGABA | AACentral | 200 | 1.8 | 4.5 | 8.5 |
| | MagT | Now/PP/SN | 667 | | | |
| | NAC.SR | Jarrow | 600 | | | |
| | Gly | Now | 1000 | | | |
| | Gln | WFoods | 500 | | | |
| T36: 8 | NGABA | AACentral | 200 | 1.5 | 8.5 | 9.5 |
| | MagT | Now/PP/SN | 1333 | | | |
| | NAC.SR | Jarrow | 600 | | | |
| | Gly | Now | 1000 | | | |
| T36: 9 | Val | NutraBio | 3150 | 2.0 | 8.5 | 9.5 |
| | MagT | Now/PP/SN | 667 | | | |
| | NAC.P | WFoods | 500 | | | |
| | Gly | Now | 1000 | | | |
| T36: 10 | NGABA | AACentral | 200 | 2.0 | 7.8 | 8.8 |
| | NAC.SR | Jarrow | 600 | | | |
| | MagT | Now/PP/SN | 1333 | | | |
| | Gly | Now | 1000 | | | |
| | Cat's Claw Bark Extract | NaturesWay | 175 | | | |
| | Cat's Claw Bark | NaturesWay | 160 | | | |
| T36: 11 | NGABA | AACentral | 200 | 1.5 | 8.5 | 9.5 |
| | MagT | Now/PP/SN | 1333 | | | |
| | NAC.P | WFoods | 500 | | | |
| | Gly | Now | 1000 | | | |
| | Boswellia | Himalaya | 250 | | | |
| T36: 12 | Val | NutraBio | 3150 | 1.5 | 8.5 | 9.5 |
| | MagT | Now/PP/SN | 1333 | | | |
| | NAC.SR | Jarrow | 600 | | | |
| | Gly | Now | 1000 | | | |
| | R-Alpha-Lipoic Acid | SwansonHP | 100 | | | |
| T36: 13 | Ile | Nutricost | 3000 | 1.0 | 7.5 | 8.8 |
| | MagT | Now/PP/SN | 1333 | | | |
| | NAC.SR | Jarrow | 600 | | | |
| | Gly | Now | 1000 | | | |
| T36: 14 | Ile | MHP | 825 | 1.0 | 6.5 | 5.5 |
| | Leu | MHP | 3300 | | | |
| | Val | MHP | 825 | | | |
| | Pyridoxine | MHP | 2.3 | | | |
| | Riboflavin | MHP | 2.6 | | | |
| | Gln | WFoods | 500 | | | |
| | MagT | Now/PP/SN | 1333 | | | |
| | NAC.SR | Jarrow | 600 | | | |
| | Gly | Now | 1000 | | | |
| T36: 15 | Leu | SNaturals | 3500 | 1.8 | 7.8 | 8.8 |
| | MagT | Now/PP/SN | 1333 | | | |
| | NAC.SR | Jarrow | 600 | | | |
| | Gly | Now | 1000 | | | |
| T36: 16 | Ile | MHP | 825 | 1.5 | 7.5 | 8.5 |
| | Leu | MHP | 3300 | | | |
| | Val | MHP | 825 | | | |
| | Pyridoxine | MHP | 2.3 | | | |
| | Riboflavin | MHP | 2.6 | | | |
| | Gln | WFoods | 500 | | | |
| | MagT | Now/PP/SN | 1333 | | | |
| | NAC.SR | Jarrow | 600 | | | |
| | Gly | Now | 1000 | | | |
| T36: 17 | NGABA | AACentral | 200 | 2.0 | 8.5 | 9.5 |
| | MagT | Now/PP/SN | 1333 | | | |
| | NAC.P | WFoods | 500 | | | |
| | Gly | Now | 1000 | | | |
| | AKG | Klaire Labs | 300 | | | |
| T36: 18 | NGABA | AACentral | 200 | 1.0 | 6.5 | 5.5 |
| | MagT | Now/PP/SN | 1333 | | | |
| | NAC.SR | Jarrow | 600 | | | |
| | Gly | Now | 1000 | | | |
| | Alpha-Lipoic Acid | SwansonHP | 200 | | | |
| T36: 19 | NGABA | AACentral | 200 | 1.0 | 6.5 | 8.5 |
| | MagT | Now/PP/SN | 1333 | | | |
| | NAC.SR | Jarrow | 600 | | | |
| | Gly | Now | 1000 | | | |
| | GSH | Jarrow | 500 | | | |
| T36: 20 | NGABA | AACentral | 200 | 1.8 | 8.8 | 9.5 |
| | MagT | DBest | 4168 | | | |
| | NAC.SR | Jarrow | 600 | | | |
| | Gly | Now | 1000 | | | |
| | Tyr | WFoods or HEB | 500 | | | |
| T36: 21 | NGABA | AACentral | 200 | 1.8 | 8.5 | 9.5 |
| | MagT | DBest | 4168 | | | |
| | NAC.SR | Jarrow | 600 | | | |
| | Gly | Now | 1000 | | | |
| | Glu | SwansonHP | 1000 | | | |
| T36: 22 | NGABA | AACentral | 200 | 1.2 | 9.5 | 8.5 |
| | MagT | DBest | 4168 | | | |
| | NAC.SR | Jarrow | 600 | | | |
| | Gly | Now | 1000 | | | |
| | Trp | WFoods | 500 | | | |
| T36: 23 | NGABA | AACentral | 200 | 1.0 | 5.5 | 6.5 |
| | MagT | DBest | 4168 | | | |
| | NAC.SR | Jarrow | 600 | | | |
| | Gly | Now | 1000 | | | |
| | GABA | PuritanP | 750 | | | |
| T36: 24 | NGABA | AACentral | 200 | 1.2 | 8.5 | 8.8 |
| | MagT | DBest | 4168 | | | |
| | NAC.SR | Jarrow | 600 | | | |
| | Gly | Now | 1000 | | | |
| | Ashwagandha | Pure Noops | 600 | | | |
| T36: 25 | NGABA | AACentral | 200 | 2.0 | 8.5 | 8.8 |
| | MagT | DBest | 4168 | | | |
| | NAC.SR | Jarrow | 600 | | | |
| | Gly | Now | 1000 | | | |
| | Thr | Douglas | 500 | | | |
| T36: 26 | NGABA | AACentral | 200 | 1.0 | 2.5 | 3.5 |
| | MagT | DBest | 4168 | | | |
| | Thr | Douglas | 500 | | | |
| T36: 27 | NGABA | AACentral | 200 | 1.2 | 8.5 | 8.8 |
| | MagT | DBest | 4168 | | | |
| | NAC.SR | Jarrow | 600 | | | |
| | Gly | Now | 1000 | | | |
| | Lemon Balm | OWH | 750 | | | |
| T36: 28 | Val | NutraBio | 3150 | 1.0 | 1.5 | 1.5 |
| | NAC.SR | Jarrow | 600 | | | |
| | Gly | Now | 1000 | | | |
| T36: 29 | NGABA | AACentral | 200 | 1.5 | 8.5 | 9.5 |
| | MagT | DBest | 2084 | | | |
| | NAC.SR | Jarrow | 600 | | | |
| | Gly | Now | 1000 | | | |
| T36: 30 | NGABA | AACentral | 200 | 2.2 | 8.8 | 7.8 |
| | NAC.SR | Jarrow | 1200 | | | |
| | MagT | DBest | 2084 | | | |
| T36: 31 | Val | NutraBio | 3150 | 2.2 | 8.8 | 8.8 |
| | MagT | DBest | 2084 | | | |
| | NAC.SR | Jarrow | 1200 | | | |
| T36: 32 | Val | NutraBio | 3150 | 1.2 | 7.8 | 7.8 |
| | MagT | DBest | 2084 | | | |
| | NAC.P | WFoods | 1000 | | | |
| T36: 33 | NAC.P | WFoods | 500 | 0.2 | 1.5 | FE |
| | Gly | Now | 1000 | | | |
| T36: 34 | Gly | Now | 2000 | 1.2 | 3.5 | 4.5 |
| T36: 35 | Val | NutraBio | 3150 | 2.2 | 9.5 | 8.8 |
| | NAC.SR | Jarrow | 600 | | | |
| | NAC.P | WFoods | 500 | | | |
| T36: 36 | NAC.SR | Jarrow | 1200 | 0.5 | 1.5 | 1.5 |
| T36: 37 | NAC.SR | Jarrow | 1200 | 2.0 | 7.8 | FE |
| | Theanine | Jarrow | 600 | | | |
| T36: 38 | NGABA | AACentral | 200 | 2.0 | 8.5 | 9.5 |
| | Theanine | Jarrow | 400 | | | |
| | NAC.SR [2 Tablets CW**] | Jarrow | 1200 | | | |
| T36: 39 | Theanine | Jarrow | 600 | 1.8 | 6.5 | FE |
| T36: 40 | NGABA | AACentral | 200 | 2.2 | 9.5 | 8.8 |
| | Theanine | Jarrow | 600 | | | |
| T36: 41 | NGABA | AACentral | 200 | 2.2 | 8.8 | 8.5 |
| | Theanine | Jarrow | 600 | | | |
| | NAC.P* | WFoods | 250 | | | |
| T36: 42 | NGABA | AACentral | 200 | 1.0 | 7.8 | 5.8 |
| | Taurine | Now | 3000 | | | |
| T36: 43 | Val | NutraBio | 3600 | 2.0 | 9.5 | 9.5 |
| | NAC.P | WFoods | 500 | | | |
| T36: 44 | Val | NutraBio | 3600 | 2.5 | 9.5 | 8.8 |
| | NAC.P* | WFoods | 250 | | | |
| T36: 45 | NGABA | AACentral | 200 | 1.5 | 8.5 | 6.8 |
| | NAC.P | WFoods | 1000 | | | |
| T36: 46 | NGABA | AACentral | 200 | 1.2 | 8.5 | 7.5 |
| | NAC.P | WFoods | 500 | | | |
| T36: 47 | NGABA | AACentral | 200 | 1.8 | 8.5 | 9.5 |
| | NAC.P | WFoods | 500 | | | |
| | Gly | Now | 1000 | | | |
| T36: 48 | NGABA | AACentral | 200 | 2.8 | 9.5 | 9.5 |
| | MagT | DBest | 3126 | | | |
| | Gly | Now | 1000 | | | |
| | NAC.P | WFoods | 500 | | | |
| T36: 49 | Val | NutraBio | 3600 | 2.2 | 9.5 | 9.8 |
| | Gly | Now | 1000 | | | |
| | NAC.P | WFoods | 500 | | | |
| T36: 50 | NGABA | AACentral | 200 | 1.8 | 8.5 | 8.8 |
| | NAC.P | WFoods | 1500 | | | |
| T36: 51 | NGABA | AACentral | 200 | 1.5 | 8.5 | 9.5 |
| | NAC.P | WFoods | 1500 | | | |
| | Gly | Now | 1000 | | | |
| T36: 52 | NGABA | AACentral | 200 | 1.5 | 8.5 | 9.5 |
| | NAC.P | WFoods | 1000 | | | |
| | Gly | Now | 1000 | | | |
| T36: 53 | NGABA | AACentral | 200 | 1.5 | 8.5 | 8.5 |
| | NAC.P | WFoods | 500 | | | |
| | Gly | Now | 1000 | | | |
| T36: 54 | NGABA | AACentral | 200 | 1.5 | 8.5 | 8.5 |
| | NAC.P* | WFoods | 250 | | | |
| | Gly | Now | 1000 | | | |
| T36: 55 | NGABA | AACentral | 200 | 3.0 | 9.5 | 9.8 |
| | NAC.P | WFoods | 500 | | | |
| | Gly | Now | 2000 | | | |
| T36: 56 | NGABA | AACentral | 200 | 1.2 | 7.5 | 8.5 |
| | NAC.P | WFoods | 500 | | | |
| | Gly | Now | 3000 | | | |
| T36: 57 | NGABA | AACentral | 200 | 1.2 | 8.8 | 9.5 |
| | NAC.P | WFoods | 500 | | | |
| | Gly | Now | 4000 | | | |
| T36: 58 | NGABA | AACentral | 200 | 1.2 | 7.5 | 4.5 |
| | NAC.P | WFoods | 1000 | | | |
| | Gly | Now | 4000 | | | |
| T36: 59 | NGABA | AACentral | 200 | 1.2 | 4.5 | 7.5 |
| | NAC.P | WFoods | 1500 | | | |
| | Gly | Now | 4000 | | | |
| T36: 60 | NGABA | AACentral | 200 | 1.2 | 7.5 | 8.5 |
| | Oleamide | Liftmode | 100 | | | |
| T36: 61 | NGABA | AACentral | 200 | 1.0 | 6.5 | 7.5 |
| T36: 62 | NGABA | AACentral | 200 | 1.5 | 8.5 | 8.5 |
| | MagT | DBest | 3126 | | | |
| T36: 63 | NGABA | AACentral | 200 | 1.5 | 7.5 | 8.5 |
| | Gly | Now | 1000 | | | |
| T36: 64 | NGABA | AACentral | 200 | 1.5 | 9.5 | 8.8 |
| | MagT | DBest | 5210 | | | |
| T36: 65 | NGABA | AACentral | 200 | 2.2 | 9.8 | 9.5 |
| | MagT | DBest | 5210 | | | |
| | NAC.P | WFoods | 500 | | | |
| T36: 66 | NGABA | AACentral | 200 | 2.2 | 8.5 | 8.8 |
| | MagT | DBest | 5210 | | | |
| | Oleamide | Liftmode | 100 | | | |
| T36: 67 | NGABA | AACentral | 200 | 2.5 | 8.5 | 9.5 |
| | MagT | DBest | 5210 | | | |
| | 5HTP | Bluebonnet | 100 | | | |
| T36: 68 | NGABA | AACentral | 200 | 1.8 | 8.5 | 8.5 |
| | Alpha-Lipoic Acid | WFoods | 500 | | | |
| T36: 69 | NGABA | AACentral | 200 | 1.8 | 8.5 | 9.5 |
| | NAC.P | WFoods | 500 | | | |
| T36: 70 | NGABA | AACentral | 200 | 1.0 | 7.5 | 5.8 |
| | AKG | Klaire Labs | 600 | | | |
| T36: 71 | NGABA | AACentral | 200 | 1.5 | 7.5 | 8.5 |
| | Grape SeedR | Radiance | 300 | | | |
| | Citrus Bioflavonoids | Radiance | 90 | | | |
| T36: 72 | NGABA | AACentral | 200 | 1.8 | 8.5 | FE |
| | Tyr | WFoods or HEB | 1000 | | | |
| T36: 73 | NGABA | AACentral | 200 | 1.2 | 7.5 | 8.5 |
| | Glu | SwansonHP | 1000 | | | |
| T36: 74 | NGABA | AACentral | 200 | 1.2 | 8.5 | 8.5 |
| | 5HTP | Bluebonnet | 200 | | | |
| T36: 75 | NGABA | AACentral | 200 | 1.2 | 7.5 | 7.5 |
| | GSH | Jarrow | 1000 | | | |
| T36: 76 | NGABA | AACentral | 200 | 1.8 | 7.5 | 7.5 |
| | Taurine | Now | 1000 | | | |
| T36: 77 | NGABA | AACentral | 200 | 1.8 | 8.5 | 8.5 |
| | Gln | WFoods | 1000 | | | |
| T36: 78 | NGABA | AACentral | 200 | 1.5 | 7.5 | 6.5 |
| | Ile | Nutricost | 4000 | | | |
| T36: 79 | NGABA | AACentral | 200 | 1.5 | 8.5 | 8.5 |
| | Leu | SNaturals | 4000 | | | |
| T36: 80 | NGABA | AACentral | 200 | 1.5 | 8.8 | 8.8 |
| | Trp | WFoods | 1000 | | | |
| T36: 81 | NGABA | AACentral | 200 | 2.2 | 9.5 | 8.5 |
| | Val | NutraBio | 3600 | | | |
| T36: 82 | NGABA | AACentral | 200 | 1.5 | 7.5 | 8.5 |
| | Inositol | Now | 1000 | | | |
| T36: 83 | NGABA | AACentral | 200 | 1.2 | 4.5 | 4.5 |
| | Taurine | Now | 1000 | | | |
| T36: 84 | NGABA | AACentral | 200 | 1.0 | 6.5 | 5.5 |
| | R-Alpha-Lipoic Acid | SwansonHP | 100 | | | |
| T36: 85 | NGABA | AACentral | 200 | 1.0 | 6.5 | 4.5 |
| | Glu | SwansonHP | 1000 | | | |
| T36: 86 | NGABA | AACentral | 200 | 1.8 | 8.5 | 7.5 |
| | MagT | DBest | 3126 | | | |
| T36: 87 | Val | NutraBio | 3600 | 1.8 | 7.8 | 7.8 |
| T36: 88 | NGABA | AACentral | 200 | 1.2 | 7.8 | 7.8 |
| | Thr | Douglas | 1000 | | | |
| T36: 89 | Val | NutraBio | 3600 | 1.5 | 5.5 | 5.5 |
| | Glu | SwansonHP | 1000 | | | |
| T36: 90 | NGABA | AACentral | 200 | 1.5 | 7.5 | 7.5 |
| | Ashwagandha | Pure Noops | 600 | | | |
| T36: 91 | Val | NutraBio | 3600 | 1.0 | 7.5 | 7.5 |
| | Inositol | Now | 1000 | | | |
| T36: 92 | Val | NutraBio | 3600 | 1.8 | 8.5 | 7.5 |
| | Alpha-Lipoic Acid | SwansonHP | 100 | | | |
| T36: 93 | NGABA | AACentral | 200 | 1.8 | 7.5 | 6.5 |
| | Lemon Balm | OWH | 750 | | | |
| T36: 94 | NGABA | AACentral | 200 | 1.8 | 8.5 | 8.5 |
| | Propolis Extract | Y.S. Farms | 1000 | | | |
| T36: 95 | Val | NutraBio | 3600 | 1.8 | 9.5 | 8.5 |
| | 5HTP | Bluebonnet | 200 | | | |
| T36: 96 | Val | NutraBio | 3600 | 1.2 | 6.5 | 6.5 |
| | GSH | Jarrow | 1000 | | | |
| T36: 97 | NGABA | AACentral | 200 | 1.2 | 7.5 | 7.5 |
| | Ala | Montiff | 1000 | | | |
| T36: 98 | NGABA | AACentral | 200 | 1.8 | 8.5 | 8.5 |
| | Boswellia | Himalaya | 250 | | | |
| T36: 99 | Val | NutraBio | 3600 | 1.5 | 8.5 | 8.5 |
| | Grape SeedR | Radiance | 200 | | | |
| | Citrus Bioflavonoids | Radiance | 60 | | | |
| T36: 100 | NGABA | AACentral | 200 | 1.8 | 8.5 | 8.5 |
| | Boswellia | Himalaya | 500 | | | |
| T36: 101 | Ketoisocaproate | Twinlab | 500 | 0.8 | 6.5 | 6.5 |
| T36: 102 | Val | NutraBio | 3600 | 1.8 | 5.5 | 5.5 |
| | NAC.P | WFoods | 500 | | | |
| | Ketoisocaproate | Twinlab | 500 | | | |
| T36: 103 | NGABA | AACentral | 200 | 1.2 | 6.5 | 6.5 |
| | Cat's Claw Bark Extract | NaturesWay | 350 | | | |
| | Cat's Claw Bark | NaturesWay | 320 | | | |
| T36: 104 | Val | NutraBio | 3600 | 1.0 | 5.5 | 5.5 |
| | Boswellia | Himalaya | 500 | | | |
| T36: 105 | NGABA | AACentral | 200 | 1.8 | 8.8 | 8.5 |
| | Cys | SwansonF1 | 1000 | | | |
| T36: 106 | NGABA | AACentral | 200 | 1.2 | 6.5 | 6.5 |
| | Met | SwansonHP | 1000 | | | |
| T36: 107 | Val | NutraBio | 3600 | 1.2 | 8.5 | 8.5 |
| | Thr | Douglas | 1000 | | | |
| T36: 108 | NGABA | AACentral | 200 | 2.0 | 8.5 | 9.5 |
| | Tocopheryl Succinate | Now | 658 | | | |
| T36: 109 | Val | NutraBio | 3600 | 1.2 | 8.8 | 8.8 |
| | Ashwagandha | Pure Noops | 600 | | | |
| T36: 110 | D-Ser | Relentless | 700 | 1.2 | 3.5 | 3.5 |
| T36: 111 | D-Ser | Relentless | 1400 | 1.5 | 4.5 | 4.5 |
| T36: 112 | Huperzine A | SwansonHP | 0.05 | 1.2 | 7.5 | 8.5 |
| T36: 113 | Galantamine HBr | Relentless | 4 | 1.8 | 5.5 | 5.5 |
| T36: 114 | DimethylGly | Now | 125 | 1.5 | 8.5 | 8.5 |
| T36: 115 | Alpha-GlyceroPhosphoCholine | SwansonHP | 300 | 1.5 | 8.5 | 8.8 |
| T36: 116 | Val | NutraBio | 3600 | 1.5 | 8.5 | 8.8 |
| | Ala | Montiff | 1000 | | | |
| T36: 117 | NGABA | AACentral | 200 | 2.0 | 5.5 | 5.5 |
| | Galantamine HBr [1 Tablet CW**] | Relentless | 4 | | | |
| T36: 118 | Val | NutraBio | 3600 | 1.2 | 7.5 | 7.5 |
| | Propolis Extract | Y.S. Farms | 1000 | | | |
| T36: 119 | NGABA | AACentral | 200 | 1.5 | 8.5 | 8.5 |
| | Alpha-GlyceroPhosphoCholine | SwansonHP | 600 | | | |
| T36: 120 | Val | NutraBio | 3600 | 1.5 | 6.5 | 6.5 |
| | AKG | Klaire Labs | 600 | | | |
| T36: 121 | Val | NutraBio | 3600 | 1.2 | 6.5 | 6.5 |
| | Leu | SNaturals | 3500 | | | |
| T36: 122 | Val | NutraBio | 3600 | 1.2 | 7.8 | 7.8 |
| | Cys | SwansonF1 | 1000 | | | |
| T36: 123 | Val | NutraBio | 3600 | 1.2 | 8.8 | 8.8 |
| | Met | SwansonHP | 1000 | | | |
| T36: 124 | NGABA | AACentral | 200 | 1.5 | 7.5 | 7.5 |
| | Alpha-Lipoic Acid | WFoods | 200 | | | |
| T36: 125 | NGABA | AACentral | 200 | 0.8 | 8.5 | 8.5 |
| | Ubiquinone | NaturesLab | 400 | | | |
| | Alpha-Lipoic Acid | NaturesLab | 600 | | | |
| | Acetyl-Carnitine | NaturesLab | 500 | | | |
| T36: 126 | NGABA | AACentral | 200 | 1.2 | 6.5 | 6.5 |
| | D-Ser | Relentless | 2800 | | | |
| T36: 127 | Val | NutraBio | 3600 | 1.8 | 8.8 | 8.8 |
| | Trp | WFoods | 1000 | | | |
| T36: 128 | NGABA | AACentral | 200 | 1.2 | 7.5 | 7.5 |
| | Ketoisocaproate | Twinlab | 2000 | | | |
| T36: 129 | NGABA | AACentral | 200 | 0.8 | 6.5 | FE |
| | Huperzine A | SwansonHP | 0.1 | | | |
| T36: 130 | Val | NutraBio | 3600 | 1.5 | 6.5 | 6.5 |
| | R-Alpha-Lipoic Acid | SwansonHP | 100 | | | |
| T36: 131 | NGABA | AACentral | 200 | 1.5 | 8.8 | 8.8 |
| | Inosine | SwansonHP | 1000 | | | |
| T36: 132 | NGABA | AACentral | 200 | 1.8 | 7.5 | 7.5 |
| | DimethylGly | Now | 250 | | | |
| T36: 133 | NGABA | AACentral | 200 | 1.2 | 8.5 | 8.5 |
| | Ile | Now | 1300 | | | |
| | Leu | Now | 3200 | | | |
| | Val | Now | 1300 | | | |
| T36: 134 | Val | NutraBio | 3600 | 1.2 | 6.5 | 6.5 |
| | Ile | Nutricost | 4000 | | | |
| T36: 135 | NGABA | AACentral | 200 | 1.2 | 8.8 | 8.5 |
| | MagT | DBest | 2084 | | | |
| T36: 136 | Val | BulkSupps | 3600 | 1.2 | 8.8 | 8.8 |
| | Ubiquinone | NaturesLab | 400 | | | |
| | Alpha-Lipoic Acid | NaturesLab | 600 | | | |
| | Acetyl-Carnitine | NaturesLab | 500 | | | |
| T36: 137 | Val | BulkSupps | 3600 | 1.2 | 8.8 | 8.8 |
| | Alpha-Lipoic Acid | WFoods | 200 | | | |
| T36: 138 | Val | BulkSupps | 3600 | 1.5 | 8.8 | 8.8 |
| | Tocopheryl Succinate | Now | 658 | | | |
| T36: 139 | Val | BulkSupps | 3600 | 1.0 | 8.8 | 8.8 |
| | Lemon Balm | OWH | 750 | | | |
| T36: 140 | Val | BulkSupps | 3600 | 0.2 | 7.8 | 7.8 |
| | Acetyl-Carnitine HCl | Jarrow | 1000 | | | |
| T36: 141 | Val | BulkSupps | 3600 | 0.5 | 5.5 | 5.5 |
| | Tyr | WFoods or HEB | 1000 | | | |
| T36: 142 | Val | BulkSupps | 3600 | 1.5 | 9.2 | 9.2 |
| | Inosine | SwansonHP | 1000 | | | |
| T36: 143 | 95% Baicalin | Liftmode | 540 | 1.2 | 6.5 | 6.5 |
| T36: 144 | 95% Honokiol/Magnolol | Liftmode | 275 | 0.2 | 4.5 | FE |
| T36: 145 | 95% Baicalin | Liftmode | 810 | 1.5 | 6.5 | 6.5 |
| T36: 146 | Pregnenolone | SwansonHP | 10 | 1.2 | 5.5 | 5.5 |
| T36: 147 | Noopept | HealthN | 30 | 1.0 | 8.8 | 8.8 |
| T36: 148 | Val | BulkSupps | 3600 | 1.2 | 8.8 | 8.8 |
| | Noopept | HealthN | 30 | | | |
| T36: 149 | Val | BulkSupps | 3600 | 1.2 | 8.8 | 8.5 |
| | Pregnenolone | SwansonHP | 10 | | | |
| T36: 150 | Val | BulkSupps | 3600 | 1.2 | 8.5 | 8.5 |
| | NAC.P | WFoods | 500 | | | |
| | Pregnenolone | SwansonHP | 10 | | | |
| T36: 151 | NGABA | AACentral | 200 | 1.5 | 9.2 | 9.2 |
| | NAC.P | WFoods | 500 | | | |
| | 95% Baicalin | Liftmode | 810 | | | |
| T36: 152 | Val | BulkSupps | 3600 | 1.2 | 7.5 | 7.5 |
| | 95% Honokiol/Magnolol | Liftmode | 550 | | | |
| T36: 153 | Val | BulkSupps | 3600 | 1.5 | 8.8 | 8.8 |
| | NAC.P | WFoods | 500 | | | |
| | MagT | DBest | 3126 | | | |
| | Lemon Balm | OWH | 1500 | | | |
| T36: 154 | St. John's Wort | BulkSupps | 543 | 0.8 | FO | FO |
| T36: 155 | Rosmarinic Acid | LifeLink | 140 | 1.2 | 1.5 | 1.5 |
| | Grape SeedL | LifeLink | 84 | | | |
| T36: 156 | NGABA | AACentral | 200 | 1.8 | 4.5 | 4.5 |
| | Rosmarinic Acid | LifeLink | 140 | | | |
| | Grape SeedL | LifeLink | 84 | | | |
| T36: 157 | Homotaurine | Supersmart | 100 | 1.8 | 9.2 | 9.2 |
| | MagT | DBest | 3126 | | | |
| T36: 158 | Agmatine | BulkSupps | 290 | 0.2 | 2.5 | 2.5 |
| T36: 159 | AcetylTyr | BulkSupps | 370 | 1.0 | 5.5 | 5.5 |
| T36: 160 | Kava | BulkSupps | 290 | 1.2 | 4.5 | 4.5 |
| T36: 161 | Val | BulkSupps | 3600 | 1.2 | 9.2 | 9.2 |
| | NGABA | AACentral | 200 | | | |
| | MagT | DBest | 3126 | | | |
| | NAC.SR | Jarrow | 600 | | | |
| | NAC.P | WFoods | 500 | | | |
| T36: 162 | Pyroglutamate | BulkSupps | 730 | 1.0 | 4.5 | 4.5 |
| T36: 163 | Mucuna Pruriens | BulkSupps | 370 | 1.2 | 6.5 | 6.5 |
| T36: 164 | Kava | BulkSupps | 290 | 0.5 | 1.5 | 1.5 |
| T36: 165 | AcetylGln | BulkSupps | 666 | 1.2 | 6.5 | 6.5 |
| T36: 166 | Norvaline | BulkSupps | 263 | 1.8 | 2.5 | 2.5 |
| T36: 167 | NGABA | AACentral | 200 | 1.8 | 8.5 | 8.5 |
| | Acety1Gln | BulkSupps | 666 | | | |
| T36: 168 | Ornithine | BulkSupps | 597 | 0.2 | 4.5 | 4.5 |
| T36: 169 | Val | BulkSupps | 4320 | 1.2 | 6.5 | 6.5 |
| | Rosmarinic Acid | LifeLink | 280 | | | |
| | Grape SeedL | LifeLink | 168 | | | |
| T36: 170 | Norvaline | BulkSupps | 542 | 1.8 | 5.5 | 5.5 |
| T36: 171 | Val | BulkSupps | 4320 | 1.5 | 8.8 | 8.5 |
| | Norvaline | BulkSupps | 542 | | | |
| T36: 172 | Norvaline | BulkSupps | 1084 | 1.5 | 6.5 | 6.5 |
| T36: 173 | Sarcosine | Brainvitamiz | 1860 | 1.2 | 4.5 | 4.5 |
| T36: 174 | Sarcosine | Brainvitamiz | 3100 | 1.8 | 7.5 | 7.5 |
| T36: 175 | Val | BulkSupps | 4320 | 1.8 | 8.8 | 8.8 |
| | NAC.P | WFoods | 500 | | | |
| | MagT | DBest | 3126 | | | |
| | Sarcosine | Brainvitamiz | 3720 | | | |
| T36: 176 | Betaine | BulkSupps | 1950 | 1.0 | 6.5 | 6.5 |
| T36: 177 | Val | BulkSupps | 4320 | 1.5 | 8.8 | 8.8 |
| | NAC.P | WFoods | 500 | | | |
| | MagT | DBest | 3126 | | | |
| | Betaine | BulkSupps | 3250 | | | |
| T36: 178 | DimethylGly | Now | 375 | 1.0 | 2.5 | 2.5 |
| T36: 179 | Val | BulkSupps | 4320 | 1.5 | 8.5 | 8.5 |
| | NAC.P | WFoods | 500 | | | |
| | MagT | DBest | 3126 | | | |
| | DimethylGly | Now | 750 | | | |
| T36: 180 | Acetyl-Carnitine HCl | Jarrow | 1500 | 1.2 | 4.5 | 4.5 |
| T36: 181 | Val | BulkSupps | 4320 | 0.5 | 7.5 | 7.5 |
| | NAC.P | WFoods | 500 | | | |
| | MagT | DBest | 3126 | | | |
| | Acetyl-Carnitine HCl | Jarrow | 3000 | | | |
| T36: 182 | D-Ser | Relentless | 7000 | 0.5 | 4.5 | 4.5 |
| T36: 183 | AcetylGln | BulkSupps | 1332 | 1.2 | 6.5 | 6.5 |
| T36: 184 | AcetylGln | BulkSupps | 1332 | 1.8 | 7.5 | 7.5 |
| | NAC.P | WFoods | 1000 | | | |
| T36: 185 | AcetylGln | BulkSupps | 1332 | 1.2 | 5.5 | 5.5 |
| | NGABA | AACentral | 200 | | | |
| T36: 186 | Leu | SNaturals | 4000 | 0.5 | 5.5 | 5.5 |
| | AcetylGln | BulkSupps | 1332 | | | |
| T36: 187 | AcetylGln | BulkSupps | 2664 | 1.0 | 5.5 | 5.5 |
| T36: 188 | Inosine | SwansonHP | 2000 | 1.0 | 6.5 | 6.5 |
| T36: 189 | Val | BulkSupps | 4320 | 1.2 | 6.5 | 6.5 |
| | NAC.P | BulkSupps | 720 | | | |
| | MagT | DBest | 3126 | | | |
| | Inosine | SwansonHP | 2000 | | | |
| T36: 190 | NGABA | AACentral | 200 | 1.2 | 7.5 | 7.5 |
| | MagT | DBest | 3126 | | | |
| | NAC.P | WFoods | 500 | | | |
| | Inosine | SwansonHP | 2000 | | | |
| T36: 191 | Theanine | Jarrow | 600 | 1.5 | 6.5 | 6.5 |
| | AcetylGln | BulkSupps | 2664 | | | |
| T36: 192 | Val | BulkSupps | 5040 | 1.0 | 8.8 | 9.2 |
| | NAC.P | BulkSupps | 720 | | | |
| | Saffron Extract | PipingRock | 177 | | | |
| T36: 193 | Panax ginseng | BulkSupps | 600 | 0.5 | 8.8 | 9.2 |
| T36: 194 | 83% Beta-Hydroxybutyrate | BulkSupps | 643 | 0.5 | 5.5 | 5.5 |
| T36: 195 | Calcium Pyruvate | BulkSupps | 899 | 0.5 | 8.5 | 8.5 |
| T36: 196 | Val | BulkSupps | 5040 | 1.0 | 9.2 | 8.8 |
| | NAC.P | BulkSupps | 720 | | | |
| | Panax ginseng | BulkSupps | 600 | | | |
| T36: 197 | Calcium Pyruvate | BulkSupps | 899 | 1.0 | 7.5 | 7.5 |
| T36: 198 | NGABA | AACentral | 200 | 1.5 | 7.8 | 7.8 |
| | Calcium Pyruvate | BulkSupps | 1798 | | | |
| T36: 199 | NGABA | AACentral | 300 | 1.2 | 9.2 | 9.2 |
| | Calcium Pyruvate | BulkSupps | 899 | | | |
| | Huperzine A | SwansonHP | 0.05 | | | |
| T36: 200 | Val | NutraBio | 3150 | 3.0 | 8.8 | 8.8 |
| | NAC.P | WFoods | 1000 | | | |
| | Gly | Now | 1000 | | | |
| T36: 201 | NGABA | AACentral | 200 | 1.8 | 7.5 | 8.8 |
| | MagT | Now/PP/SN | 667 | | | |
| T36: 202 | NGABA | AACentral | 200 | 3.0 | 8.8 | 8.8 |
| | MagT | Now/PP/SN | 667 | | | |
| | NAC.P | WFoods | 1000 | | | |
| | Gly | Now | 1000 | | | |
| T36: 203 | NGABA | AACentral | 200 | 1.5 | 7.8 | 9.5 |
| | MagT | Now/PP/SN | 667 | | | |
| | Ashwagandha | Pure Noops | 300 | | | |
| T36: 204 | Val | NutraBio | 1800 | 2.0 | 7.8 | 9.5 |
| | NGABA | AACentral | 200 | | | |
| | MagT | Now/PP/SN | 667 | | | |
| T36: 205 | MagT | Now/PP/SN | 667 | 1.0 | 4.8 | 7.8 |
| | NAC.P | WFoods | 1000 | | | |
| | Gly | Now | 1000 | | | |
| T36: 206 | Ile | Nutricost | 3000 | 1.0 | 6.8 | 8.8 |
| | MagT | Now/PP/SN | 667 | | | |
| | NAC.P | WFoods | 1000 | | | |
| | Gly | Now | 1000 | | | |
| T36: 207 | Ile | Nutricost | 3000 | 1.0 | 6.8 | 9.5 |
| | MagT | Now/PP/SN | 1333 | | | |
| T36: 208 | Ile | Nutricost | 3000 | 2.0 | 8.8 | 8.8 |
| | MagT | Now/PP/SN | 667 | | | |
| | NAC.P | WFoods | 1000 | | | |
| | Gly | Now | 1000 | | | |
| | 5HTP | Bluebonnet | 100 | | | |
| | Ashwagandha | Pure Noops | 300 | | | |
| T36: 209 | Val | NutraBio | 3150 | 5.0 | 9.5 | 8.5 |
| | NAC.P | WFoods | 1000 | | | |
| | MagT | Now/PP/SN | 1333 | | | |
| | Taurine | Now | 1000 | | | |
| T36: 210 | Val | NutraBio | 3150 | 1.5 | 9.5 | 8.8 |
| | NAC.P | WFoods | 1000 | | | |
| | MagT | Now/PP/SN | 667 | | | |
| | Acetyl-Carnitine HCl | Jarrow | 500 | | | |
| T36: 211 | Val | NutraBio | 3150 | 1.0 | 6.8 | 8.8 |
| | NAC.P | WFoods | 1000 | | | |
| | MagT | Now/PP/SN | 667 | | | |
| | Gly | Now | 1000 | | | |
| | Taurine | Now | 1000 | | | |
| T36: 212 | Val | NutraBio | 3150 | 4.0 | 9.5 | 9.5 |
| | NAC.P | WFoods | 500 | | | |
| | MagT | Now/PP/SN | 667 | | | |
| | Ashwagandha | Pure Noops | 300 | | | |
| | 5HTP | Bluebonnet | 100 | | | |
| | Gly | Now | 1000 | | | |
| T36: 213 | NGABA | AACentral | 200 | 2.0 | 7.8 | 8.8 |
| | MagT | Now/PP/SN | 667 | | | |
| | Gly | Now | 1000 | | | |
| T36: 214 | Val | NutraBio | 3150 | 1.5 | 7.8 | 8.8 |
| | MagT | Now/PP/SN | 667 | | | |
| | Gly | Now | 1000 | | | |
| T36: 215 | Val | NutraBio | 3150 | 1.5 | 8.5 | 9.5 |
| | MagT | Now/PP/SN | 667 | | | |
| | NAC.P | WFoods | 500 | | | |
| T36: 216 | NGABA | AACentral | 200 | 1.8 | 8.8 | 8.8 |
| | MagT | Now/PP/SN | 667 | | | |
| | NAC.P | WFoods | 500 | | | |
| | Gly | Now | 1000 | | | |
| T36: 217 | Val | NutraBio | 3150 | 2.0 | 8.8 | 8.8 |
| | MagT | Now/PP/SN | 667 | | | |
| | 5HTP | Bluebonnet | 100 | | | |
| T36: 218 | Val | NutraBio | 3150 | 4.0 | 9.5 | 9.8 |
| | MagT | Now/PP/SN | 667 | | | |
| | NAC.P | WFoods | 500 | | | |
| | 5HTP | Bluebonnet | 100 | | | |
| T36: 219 | MagT | Now/PP/SN | 667 | 0.5 | 3.5 | 8.5 |
| | NAC.P | WFoods | 500 | | | |
| T36: 220 | Val | NutraBio | 3150 | 4.0 | 8.8 | 9.5 |
| | NAC.SR | Jarrow | 600 | | | |
| | MagT | Now/PP/SN | 667 | | | |
| | Gly | Now | 1000 | | | |
| T36: 221 | Saffron Extract | PipingRock | 45 | 0.8 | 4.5 | 4.5 |
| T36: 222 | Saffron Extract | PipingRock | 89 | 1.0 | 4.5 | 4.5 |
| T36: 223 | Saffron Extract | PipingRock | 133 | 1.8 | 6.5 | 6.5 |
| T36: 224 | NGABA | LifeLink | 250 | 1.2 | 7.5 | 7.5 |
| | Saffron Extract | PipingRock | 45 | | | |
| T36: 225 | NGABA | LifeLink | 250 | 1.0 | 8.5 | 8.5 |
| | Saffron Extract | PipingRock | 89 | | | |
| T36: 226 | Val | BulkSupps | 3600 | 1.5 | 8.5 | 8.5 |
| | Saffron Extract | PipingRock | 89 | | | |
| T36: 227 | Val | BulkSupps | 4320 | 1.8 | 8.8 | 9.2 |
| | NAC.P | BulkSupps | 720 | | | |
| | Saffron Extract | PipingRock | 177 | | | |
| T36: 228 | Val | BulkSupps | 4320 | 1.8 | 8.8 | 9.2 |
| | NAC.P | BulkSupps | 720 | | | |
| | Saffron Extract | PipingRock | 177 | | | |
| | MagT | Now/PP/SN | 2000 | | | |
| T36: 229 | Val | BulkSupps | 4320 | 1.0 | 8.8 | 9.2 |
| | NAC.P | BulkSupps | 720 | | | |
| | Saffron Extract | PipingRock | 177 | | | |
| | Theanine | BulkSupps | 410 | | | |
| T36: 230 | NGABA | AACentral | 300 | 1.8 | 9.2 | 9.2 |
| | NAC.P | BulkSupps | 720 | | | |
| | Saffron Extract | PipingRock | 177 | | | |
| T36: 231 | NGABA | LifeLink | 300 | 1.8 | 9.2 | 9.2 |
| | MagT | Now/PP/SN | 2000 | | | |
| | NAC.P | BulkSupps | 720 | | | |
| | Kava | BulkSupps | 580 | | | |
| | Saffron Extract | PipingRock | 177 | | | |
| | Huperzine A | SwansonHP | 0.1 | | | |
| T36: 232 | Kava | BulkSupps | 580 | 1.8 | 9.2 | 9.2 |
| | Saffron Extract | PipingRock | 177 | | | |
| | Val | BulkSupps | 4320 | | | |
| | MagT | Now/PP/SN | 2000 | | | |
| | NAC.P | BulkSupps | 720 | | | |
| | Huperzine A | SwansonHP | 0.1 | | | |
| T36: 233 | NGABA | AACentral | 300 | 1.8 | 7.5 | 7.5 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | D-Ser | Relentless | 700 | | | |
| T36: 234 | Val | BulkSupps | 4320 | 1.8 | 9.2 | 9.2 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | Mucuna Pruriens | BulkSupps | 690 | | | |
| T36: 235 | NGABA | AACentral | 300 | 1.8 | 7.5 | 7.5 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | Mucuna Pruriens | BulkSupps | 690 | | | |
| T36: 236 | Val | BulkSupps | 4320 | 1.8 | 8.2 | 8.2 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | D-Ser | Relentless | 700 | | | |
| T36: 237 | NGABA | AACentral | 300 | 1.8 | 9.5 | 9.5 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | AcetylTyr | BulkSupps | 1380 | | | |
| T36: 238 | Val | BulkSupps | 4320 | 1.8 | 7.8 | 7.8 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | AcetylTyr | BulkSupps | 1380 | | | |
| T36: 239 | NGABA | AACentral | 300 | 0.5 | 8.2 | 8.2 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | 95% Baicalin | Liftmode | 540 | | | |
| T36: 240 | NGABA | AACentral | 300 | 0.8 | 6.5 | 6.5 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Potassium Aspartate | Bluebonnet | 198 | | | |
| T36: 241 | NGABA | AACentral | 300 | 0.8 | 8.8 | 9.2 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Pyroglutamate | BulkSupps | 730 | | | |
| T36: 242 | Val | BulkSupps | 4320 | 0.5 | 8.5 | 8.8 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Pyroglutamate | BulkSupps | 730 | | | |
| | Beta-Hydroxy Beta-Methylbutyrate | Now | 2000 | 1.8 | 4.5 | 4.5 |
| T36: 243 | Calcium | Now | 240 | | | |
| T36: 244 | Arachidonic Acid | ElementN | 700 | 1.0 | 5.5 | 5.5 |
| T36: 245 | Valerian | WFoods | 1000 | 0.5 | 8.8 | 8.8 |
| T36: 246 | Sodium Butyrate | Bodybio | 1513 | 0.8 | 6.5 | 6.5 |
| T36: 247 | Cinchona | Terra Vita | 400 | 0.5 | 8.5 | 8.5 |
| T36: 248 | 80% Genistein | VitalN | 500 | 0.8 | 7.5 | 7.5 |
| T36: 249 | Harmala | Amalth | 500 | 1.2 | 6.5 | 6.5 |
| T36: 250 | Meclizine | Reliable | 12.5 | 0.5 | 7.5 | 7.5 |
| T36: 251 | Loperamide Hydrochloride | J&J | 2 | 1.0 | 8.8 | 8.8 |
| T36: 252 | Loperamide Hydrochloride | J&J | 2 | 1.0 | 6.5 | 6.5 |
| T36: 253 | Collagen Peptides | Vital Proteins | 2944 | 2.5 | 9.2 | 9.2 |
| | NGABA | LifeLink | 200 | | | |
| | NAC.P | BulkSupps | 720 | | | |
| | Saffron Extract | PipingRock | 177 | | | |
| T36: 254 | Phenylethylamine | Liftmode | 208 | 0.5 | 3.5 | 2.5 |
| T36: 255 | Fasoracetam | NoopDepot | 33 | 0.2 | 2.5 | 2.5 |
| T36: 256 | Citicoline | Liftmode | 208 | 1.0 | 7.5 | 7.5 |
| | Val | NutraBio | 3150 | 1.5 | 8.5 | 9.8 |
| | MagT | Now/PP/SN | 1333 | | | |
| | NAC.SR | Jarrow | 600 | | | |
| | Gly | Now | 1000 | | | |
| T36: 257 | Yohimbe Bark | SwansonF1 | 600 | | | |
| T36: 258 | NGABA | AACentral | 200 | 1.0 | 8.5 | 9.5 |
| | MagT | Now/PP/SN | 1333 | | | |
| | NAC.SR | Jarrow | 600 | | | |
| | Gly | Now | 1000 | | | |
| | Lemon Balm | OWH | 750 | | | |
| T36: 259 | NGABA | AACentral | 200 | 1.5 | 7.8 | 8.8 |
| | MagT | Now/PP/SN | 1333 | | | |
| | NAC.SR | Jarrow | 600 | | | |
| | Gly | Now | 1000 | | | |
| | Grape SeedR | Radiance | 300 | | | |
| | Citrus Bioflavonoids | Radiance | 90 | | | |
| T36: 260 | NGABA | AACentral | 200 | 1.0 | 8.5 | 7.5 |
| | MagT | DBest | 4168 | | | |
| | NAC.SR | Jarrow | 600 | | | |
| | Gly | Now | 1000 | | | |
| | R-Alpha-Lipoic Acid | SwansonHP | 100 | | | |
| T36: 261 | NGABA | AACentral | 200 | 1.8 | 8.5 | 9.5 |
| | MagT | DBest | 4168 | | | |
| | NAC.SR | Jarrow | 600 | | | |
| | Gly | Now | 1000 | | | |
| | Yohimbe Bark | SwansonF1 | 600 | | | |
| T36: 262 | NGABA | AACentral | 200 | 1.2 | 8.5 | 8.5 |
| | GABA | PuritanP | 1500 | | | |
| T36: 263 | NGABA | AACentral | 200 | 1.2 | 4.5 | 1.5 |
| | Alpha-Lipoic Acid | SwansonHP | 250 | | | |
| T36: 264 | NGABA | AACentral | 200 | 1.2 | 9.5 | 9.8 |
| | Yohimbe Bark | SwansonF1 | 600 | | | |
| T36: 265 | NGABA | AACentral | 200 | 1.5 | 7.5 | 7.5 |
| | Acetyl-Carnitine HCI | Jarrow | 1000 | | | |
| T36: 266 | NGABA | AACentral | 200 | 1.0 | 4.5 | 4.5 |
| | Beta-Ala | Now | 1500 | | | |
| T36: 267 | Inosine | SwansonHP | 500 | 1.2 | 7.5 | 7.5 |
| T36: 268 | Val | NutraBio | 3600 | 2.0 | 9.5 | 9.5 |
| | MagT | DBest | 3126 | | | |
| | NAC.P | WFoods | 500 | | | |
| | Galantamine HBr [1 of 2 Tablets | Relentless | 8 | | | |
| | CW**] | | | | | |
| T36: 269 | Val | NutraBio | 3600 | 1.2 | 8.8 | 8.5 |
| | Yohimbe Bark | SwansonF1 | 600 | | | |
| T36: 270 | Val | NutraBio | 3600 | 1.8 | 8.8 | 8.8 |
| | MagT | DBest | 3126 | | | |
| | NAC.P | WFoods | 500 | | | |
| | NAC.SR | Jarrow | 600 | | | |
| | Galantamine HBr | Relentless | 12 | | | |
| T36: 271 | Val | NutraBio | 3600 | 1.8 | 9.5 | 10.5 |
| | MagT | DBest | 3126 | | | |
| | NAC.P | WFoods | 500 | | | |
| | D-Ser | Relentless | 700 | | | |
| | DimethylGly | Now | 125 | | | |
| | Galantamine HBr | Relentless | 4 | | | |
| | Val | BulkSupps | 3600 | 1.2 | 9.2 | 9.2 |
| T36: 272 | GABA | PuritanP | 1500 | | | |
| T36: 273 | 95% Honokiol/Magnolol | Liftmode | 550 | 1.0 | 0.0 | 0.0 |
| T36: 274 | Homotaurine | Supersmart | 100 | 1.2 | FO | FO |
| | NAC.P | WFoods | 500 | | | |
| T36: 275 | Sarcosine | Brainvitamiz | 1240 | 1.2 | 1.5 | 2.5 |
| T36: 276 | Val | BulkSupps | 5040 | 0.5 | 9.5 | 9.5 |
| | Calcium Pyruvate | BulkSupps | 1798 | | | |
| T36: 277 | Val | BulkSupps | 4320 | 2.0 | 8.8 | 8.8 |
| | MagT | Now/PP/SN | 2000 | | | |
| | NAC.P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Lemon Balm | OWH | 750 | | | |
| T36: 278 | Sarpagandha | Amalth | 250 | 1.5 | 8.5 | 8.5 |
| T36: 279 | Dextromethorphan | TopCare | 30 | 0.8 | 7.8 | 7.8 |
| T36: 280 | Pramiracetam | NoopDepot | 125 | 0.5 | 6.5 | 6.5 |
| T36: 281 | Phenylpiracetam | NoopDepot | 150 | 0.5 | 1.5 | 1.5 |
| T36: 282 | Val | BulkSupps | 5040 | 1.0 | 7.8 | 7.8 |
| | NAC.P | BulkSupps | 720 | | | |
| | Citicoline | Liftmode | 208 | | | |

*About half of the powder removed from capsule;
**“Tablet(s) CW” means the tablet(s) were chewed before swallowing to promote rapid absorption

TABLE 36B
Treatment Substance Ingested with Water in the Afternoon

| Example | C | CT | A | B | BM | BS | D | DE |
|---|---|---|---|---|---|---|---|---|
| T36: 1 | 3.5 | No | 8.5 | 8.8 | N/A | N/A | 3.0 | 1.0 |
| T36: 2 | 1.5 | No | 5.8 | 8.8 | N/A | N/A | 3.0 | 2.0 |
| T36: 3 | 1.5 | No | 5.5 | 8.5 | N/A | N/A | 3.0 | 3.0 |
| T36: 4 | 1.5 | No | 3.5 | 7.5 | N/A | N/A | 4.0 | 3.0 |
| T36: 5 | 1.0 | No | 4.5 | 3.5 | N/A | N/A | 3.0 | 1.0 |
| T36: 6 | 1.5 | No | 4.5 | 7.5 | N/A | N/A | 3.0 | 2.0 |
| T36: 7 | 1.8 | No | 4.5 | 8.5 | N/A | N/A | 3.0 | 2.0 |
| T36: 8 | 1.5 | No | 8.5 | 9.5 | N/A | N/A | 3.0 | 2.0 |
| T36: 9 | 2.0 | No | 8.5 | 9.5 | N/A | N/A | 3.0 | 2.0 |
| T36: 10 | 2.0 | No | 7.8 | 8.8 | N/A | N/A | 3.0 | 2.0 |
| T36: 11 | 1.5 | No | 8.5 | 9.5 | N/A | N/A | 3.0 | 2.0 |
| T36: 12 | 1.5 | No | 8.5 | 9.5 | N/A | N/A | 3.0 | 2.0 |
| T36: 13 | 1.0 | No | 7.5 | 8.8 | N/A | N/A | 3.0 | 2.0 |
| T36: 14 | 1.0 | No | 6.5 | 5.5 | N/A | N/A | 3.0 | 2.0 |
| T36: 15 | 1.8 | No | 7.8 | 8.8 | N/A | N/A | 3.0 | 2.0 |
| T36: 16 | 1.5 | No | 7.5 | 8.5 | N/A | N/A | 3.0 | 2.0 |
| T36: 17 | 2.0 | No | 8.5 | 9.5 | N/A | N/A | 3.0 | 2.0 |
| T36: 18 | 1.0 | No | 6.5 | 5.5 | N/A | N/A | 3.0 | 2.0 |
| T36: 19 | 1.0 | No | 6.5 | 8.5 | N/A | N/A | 3.0 | 2.0 |
| T36: 20 | 1.8 | No | 8.8 | 9.5 | N/A | N/A | 3.0 | 3.0 |
| T36: 21 | 1.8 | No | 8.5 | 9.5 | N/A | N/A | 3.0 | 2.0 |
| T36: 22 | 1.2 | No | 9.5 | 8.5 | 8.5 | N/A | 3.0 | 2.0 |
| T36: 23 | 1.0 | No | 5.5 | 6.5 | N/A | N/A | 3.0 | 1.0 |
| T36: 24 | 1.2 | No | 8.5 | 8.8 | N/A | N/A | 3.0 | 2.0 |
| T36: 25 | 2.0 | No | 8.5 | 8.8 | N/A | N/A | 3.0 | 2.0 |
| T36: 26 | 1.0 | No | 2.5 | 3.5 | N/A | N/A | 3.0 | 2.0 |
| T36: 27 | 1.2 | No | 8.5 | 8.8 | 8.8 | N/A | 3.0 | 2.0 |
| T36: 28 | 1.0 | No | 1.5 | 1.5 | N/A | N/A | 3.0 | 2.0 |
| T36: 29 | 1.5 | No | 8.5 | 9.5 | N/A | N/A | 3.0 | 2.0 |
| T36: 30 | 2.2 | No | 8.8 | 7.8 | N/A | N/A | 3.0 | 2.0 |
| T36: 31 | 2.2 | No | 8.8 | 8.8 | N/A | N/A | 3.0 | 1.0 |
| T36: 32 | 1.2 | No | 7.8 | 7.8 | N/A | N/A | 3.0 | 2.0 |
| T36: 33 | 0.2 | No | 1.5 | FE | N/A | N/A | FE | FE |
| T36: 34 | 1.2 | No | 3.5 | 4.5 | N/A | N/A | 3.0 | 1.0 |
| T36: 35 | 2.2 | No | 9.5 | 8.8 | N/A | N/A | 3.0 | 2.0 |
| T36: 36 | 0.5 | No | 1.5 | 1.5 | N/A | N/A | 3.0 | 2.0 |
| T36: 37 | 2.0 | No | 7.8 | FE | N/A | N/A | FE | FE |
| T36: 38 | 2.0 | No | 8.5 | 9.5 | 7.5 | N/A | 3.0 | 2.0 |
| T36: 39 | 1.8 | No | 6.5 | FE | N/A | N/A | FE | FE |
| T36: 40 | 2.2 | No | 9.5 | 8.8 | N/A | N/A | 3.0 | 2.0 |
| T36: 41 | 2.2 | No | 8.8 | 8.5 | N/A | N/A | 3.0 | 2.0 |
| T36: 42 | 1.0 | No | 7.8 | 5.8 | N/A | N/A | 1.0 | 2.0 |
| T36: 43 | 2.0 | No | 9.5 | 9.5 | N/A | N/A | 3.0 | 2.0 |
| T36: 44 | 2.5 | No | 9.5 | 8.8 | N/A | N/A | 3.0 | 2.0 |
| T36: 45 | 1.5 | No | 8.5 | 6.8 | N/A | N/A | 3.0 | 2.0 |
| T36: 46 | 1.2 | No | 8.5 | 7.5 | N/A | N/A | 3.0 | 2.0 |
| T36: 47 | 1.8 | No | 8.5 | 9.5 | N/A | N/A | 3.0 | 2.0 |
| T36: 48 | 2.8 | No | 9.5 | 9.5 | N/A | N/A | 3.0 | 2.0 |
| T36: 49 | 2.2 | No | 9.5 | 9.8 | N/A | N/A | 3.0 | 2.0 |
| T36: 50 | 1.8 | No | 8.5 | 8.8 | N/A | N/A | 3.0 | 2.0 |
| T36: 51 | 1.5 | No | 8.5 | 9.5 | N/A | N/A | 1.5 | 2.0 |
| T36: 52 | 1.5 | No | 8.5 | 9.5 | 9.5 | N/A | 3.0 | 2.0 |
| T36: 53 | 1.5 | No | 8.5 | 8.5 | N/A | N/A | 3.0 | 1.0 |
| T36: 54 | 1.5 | No | 8.5 | 8.5 | N/A | N/A | 3.0 | 1.0 |
| T36: 55 | 3.0 | No | 9.5 | 9.8 | 7.5 | N/A | 3.0 | 2.0 |
| T36: 56 | 1.2 | No | 7.5 | 8.5 | N/A | N/A | 2.0 | 1.0 |
| T36: 57 | 1.2 | No | 8.8 | 9.5 | N/A | N/A | 3.0 | 2.0 |
| T36: 58 | 1.2 | No | 7.5 | 4.5 | 4.5 | N/A | 2.0 | 1.0 |
| T36: 59 | 1.2 | No | 4.5 | 7.5 | N/A | N/A | 3.0 | 2.0 |
| T36: 60 | 1.2 | No | 7.5 | 8.5 | 4.5 | FT | 3.0 | 2.0 |
| T36: 61 | 1.0 | No | 6.5 | 7.5 | N/A | N/A | 3.0 | 2.0 |
| T36: 62 | 1.5 | No | 8.5 | 8.5 | N/A | N/A | 3.0 | 2.0 |
| T36: 63 | 1.5 | No | 7.5 | 8.5 | N/A | N/A | 3.0 | 2.0 |
| T36: 64 | 1.5 | No | 9.5 | 8.8 | 7.5 | N/A | 3.0 | 2.0 |
| T36: 65 | 2.2 | No | 9.8 | 9.5 | 8.5 | N/A | 2.0 | 2.0 |
| T36: 66 | 2.2 | No | 8.5 | 8.8 | N/A | N/A | 3.0 | 2.0 |
| T36: 67 | 2.5 | No | 8.5 | 9.5 | N/A | N/A | 3.5 | 2.0 |
| T36: 68 | 1.8 | No | 8.5 | 8.5 | 0.0 | SL | 3.0 | 1.0 |
| T36: 69 | 1.8 | No | 8.5 | 9.5 | N/A | N/A | 3.0 | 2.0 |
| T36: 70 | 1.0 | No | 7.5 | 5.8 | 0.0 | N/A | 3.0 | 2.0 |
| T36: 71 | 1.5 | No | 7.5 | 8.5 | N/A | N/A | 3.0 | 2.0 |
| T36: 72 | 1.8 | No | 8.5 | FE | N/A | N/A | FE | FE |
| T36: 73 | 1.2 | No | 7.5 | 8.5 | N/A | N/A | 3.0 | 2.0 |
| T36: 74 | 1.2 | No | 8.5 | 8.5 | N/A | N/A | 3.0 | 2.0 |
| T36: 75 | 1.2 | No | 7.5 | 7.5 | 4.5 | N/A | 3.0 | 2.0 |
| T36: 76 | 1.8 | No | 7.5 | 7.5 | 0.0 | N/A | 3.0 | 2.0 |
| T36: 77 | 1.8 | No | 8.5 | 8.5 | 0.0 | N/A | 3.0 | 2.0 |
| T36: 78 | 1.5 | No | 7.5 | 6.5 | N/A | N/A | 3.0 | 2.0 |
| T36: 79 | 1.5 | No | 8.5 | 8.5 | 4.5 | N/A | 3.0 | 1.0 |
| T36: 80 | 1.5 | No | 8.8 | 8.8 | 3.5 | N/A | 3.0 | 2.0 |
| T36: 81 | 2.2 | No | 9.5 | 8.5 | 6.5 | N/A | 3.0 | 2.0 |
| T36: 82 | 1.5 | No | 7.5 | 8.5 | 0.0 | N/A | 3.0 | 2.0 |
| T36: 83 | 1.2 | No | 4.5 | 4.5 | N/A | N/A | 3.0 | 2.0 |
| T36: 84 | 1.0 | No | 6.5 | 5.5 | N/A | N/A | 3.0 | 1.0 |
| T36: 85 | 1.0 | No | 6.5 | 4.5 | 0.0 | N/A | 3.0 | 2.0 |
| T36: 86 | 1.8 | No | 8.5 | 7.5 | 4.5 | N/A | 3.0 | 2.0 |
| T36: 87 | 1.8 | No | 7.8 | 7.8 | 0.0 | N/A | 3.0 | 2.0 |
| T36: 88 | 1.2 | No | 7.8 | 7.8 | 0.0 | N/A | 3.0 | 2.0 |
| T36: 89 | 1.5 | No | 5.5 | 5.5 | N/A | N/A | 2.0 | 1.0 |
| T36: 90 | 1.5 | No | 7.5 | 7.5 | 0.0 | N/A | 3.0 | 1.0 |
| T36: 91 | 1.0 | No | 7.5 | 7.5 | N/A | N/A | 2.0 | 1.0 |
| T36: 92 | 1.8 | No | 8.5 | 7.5 | 0.0 | N/A | 2.5 | 2.0 |
| T36: 93 | 1.8 | No | 7.5 | 6.5 | 0.0 | N/A | 3.0 | 2.0 |
| T36: 94 | 1.8 | No | 8.5 | 8.5 | 0.0 | N/A | 3.0 | 2.0 |
| T36: 95 | 1.8 | No | 9.5 | 8.5 | 0.0 | N/A | 2.0 | 2.0 |
| T36: 96 | 1.2 | No | 6.5 | 6.5 | N/A | N/A | 3.0 | 1.0 |
| T36: 97 | 1.2 | No | 7.5 | 7.5 | N/A | N/A | 3.0 | 2.0 |
| T36: 98 | 1.8 | No | 8.5 | 8.5 | 8.5 | N/A | 3.0 | 2.0 |
| T36: 99 | 1.5 | No | 8.5 | 8.5 | 4.5 | N/A | 3.0 | 2.0 |
| T36: 100 | 1.8 | No | 8.5 | 8.5 | 4.5 | N/A | 3.0 | 2.0 |
| T36: 101 | 0.8 | No | 6.5 | 6.5 | 6.5 | N/A | 3.0 | 2.0 |
| T36: 102 | 1.8 | No | 6.5 | 5.5 | N/A | N/A | 3.0 | 2.0 |
| T36: 103 | 1.2 | No | 6.5 | 6.5 | 3.5 | N/A | 2.5 | 2.0 |
| T36: 104 | 1.0 | No | 5.5 | 5.5 | 0.0 | N/A | 3.0 | 2.0 |
| T36: 105 | 1.8 | No | 8.8 | 8.5 | 3.5 | N/A | 1.0 | 2.0 |
| T36: 106 | 1.2 | No | 6.5 | 6.5 | 0.0 | N/A | 2.5 | 2.0 |
| T36: 107 | 1.2 | No | 8.5 | 8.5 | 0.0 | N/A | 2.0 | 1.0 |
| T36: 108 | 2.0 | No | 8.5 | 9.5 | N/A | N/A | 3.0 | 2.0 |
| T36: 109 | 1.2 | No | 8.8 | 8.8 | 3.5 | N/A | 2.5 | 2.0 |
| T36: 110 | 1.2 | No | 3.5 | 3.5 | N/A | N/A | 3.0 | 2.0 |
| T36: 111 | 1.5 | No | 4.5 | 4.5 | N/A | N/A | 3.0 | 2.0 |
| T36: 112 | 1.2 | No | 7.5 | 8.5 | 6.5 | N/A | 2.5 | 2.0 |
| T36: 113 | 1.8 | No | 5.5 | 5.5 | 0.0 | N/A | 2.0 | 2.0 |
| T36: 114 | 1.5 | No | 8.5 | 8.5 | N/A | N/A | 3.0 | 2.0 |
| T36: 115 | 1.5 | No | 8.5 | 8.8 | N/A | N/A | 3.0 | 2.0 |
| T36: 116 | 1.5 | No | 8.5 | 8.8 | N/A | N/A | 3.0 | 2.0 |
| T36: 117 | 2.0 | No | 5.5 | 5.5 | 0.0 | N/A | 3.0 | 2.0 |
| T36: 118 | 1.2 | No | 7.5 | 7.5 | 0.0 | N/A | 3.0 | 2.0 |
| T36: 119 | 1.5 | No | 8.5 | 8.5 | 0.0 | SL | 3.5 | 2.0 |
| T36: 120 | 1.5 | No | 6.5 | 6.5 | 0.0 | N/A | 2.5 | 2.0 |
| T36: 121 | 1.2 | No | 6.5 | 5.5 | N/A | N/A | 2.5 | 2.0 |
| T36: 122 | 1.2 | No | 7.8 | 7.8 | 5.5 | N/A | 2.5 | 2.0 |
| T36: 123 | 1.2 | No | 8.8 | 8.8 | 8.8 | N/A | 3.0 | 2.0 |
| T36: 124 | 1.5 | No | 7.5 | 7.5 | 0.0 | N/A | 2.5 | 2.0 |
| T36: 125 | 0.8 | No | 8.5 | 8.5 | 0.0 | N/A | 2.0 | 2.0 |
| T36: 126 | 1.2 | No | 6.5 | 6.5 | N/A | N/A | 3.0 | 2.0 |
| T36: 127 | 1.8 | Yes | 8.8 | 8.8 | 0.0 | N/A | 3.0 | 2.0 |
| T36: 128 | 1.2 | No | 7.5 | 7.5 | N/A | N/A | 2.5 | 2.0 |
| T36: 129 | 0.8 | No | 6.5 | FE | N/A | N/A | FE | FE |
| T36: 130 | 1.5 | No | 6.5 | 6.5 | 0.0 | N/A | 2.5 | 2.0 |
| T36: 131 | 1.5 | No | 8.8 | 8.8 | 6.5 | N/A | 3.5 | 2.0 |
| T36: 132 | 1.8 | No | 7.5 | 7.5 | 0.0 | N/A | 2.5 | 2.0 |
| T36: 133 | 1.2 | No | 8.5 | 8.5 | N/A | N/A | 3.0 | 2.0 |
| T36: 134 | 1.2 | No | 6.5 | 6.5 | 2.5 | N/A | 3.0 | 2.0 |
| T36: 135 | 1.2 | No | 8.8 | 8.5 | 6.5 | N/A | 3.0 | 2.0 |
| T36: 136 | 1.2 | No | 8.8 | 8.8 | N/A | N/A | 3.0 | 2.0 |
| T36: 137 | 1.2 | No | 8.8 | 8.8 | N/A | N/A | 3.0 | 2.0 |
| T36: 138 | 1.5 | No | 8.8 | 8.8 | 2.5 | N/A | 2.0 | 2.0 |
| T36: 139 | 1.0 | No | 8.8 | 8.8 | N/A | N/A | 2.0 | 1.0 |
| T36: 140 | 0.2 | No | 7.8 | 7.8 | 3.5 | N/A | 3.0 | 2.0 |
| T36: 141 | 0.5 | No | 5.5 | 5.5 | 1.5 | N/A | 2.5 | 2.0 |
| T36: 142 | 1.5 | No | 9.2 | 9.2 | 0.0 | IS | 2.0 | 2.0 |
| T36: 143 | 1.2 | No | 6.5 | 6.5 | 6.5 | N/A | 3.0 | 2.0 |
| T36: 144 | 0.2 | No | 4.5 | FE | N/A | N/A | FE | FE |
| T36: 145 | 1.5 | No | 6.5 | 6.5 | 6.5 | N/A | 3.0 | 2.0 |
| T36: 146 | 1.2 | No | 5.5 | 5.5 | 5.5 | N/A | 3.0 | 2.0 |
| T36: 147 | 1.0 | No | 8.8 | 8.8 | 0.0 | IS | 2.5 | 2.0 |
| T36: 148 | 1.2 | No | 8.8 | 8.8 | 5.5 | N/A | 3.0 | 2.0 |
| T36: 149 | 1.2 | No | 8.8 | 8.5 | 5.5 | N/A | 3.0 | 2.0 |
| T36: 150 | 1.2 | No | 8.5 | 8.5 | 5.5 | N/A | 3.0 | 2.0 |
| T36: 151 | 1.5 | No | 9.2 | 9.2 | 8.5 | N/A | 3.0 | 3.0 |
| T36: 152 | 1.2 | No | 7.5 | 7.5 | 4.5 | N/A | 3.0 | 2.0 |
| T36: 153 | 1.5 | No | 8.8 | 8.8 | 4.5 | N/A | 3.0 | 2.0 |
| T36: 154 | 0.8 | No | FO | FO | N/A | N/A | FO | FO |
| T36: 155 | 1.2 | No | 1.5 | 1.5 | 1.5 | N/A | 3.0 | 2.0 |
| T36: 156 | 1.8 | No | 4.5 | 4.5 | 0.0 | N/A | 3.0 | 2.0 |
| T36: 157 | 1.8 | No | 9.2 | 9.2 | 3.5 | N/A | 3.0 | 2.0 |
| T36: 158 | 0.2 | No | 2.5 | 2.5 | N/A | N/A | 1.0 | 2.0 |
| T36: 159 | 1.0 | No | 5.5 | 5.5 | 5.5 | N/A | 2.0 | 2.0 |
| T36: 160 | 1.2 | No | 4.5 | 4.5 | 4.5 | N/A | 1.0 | 2.0 |
| T36: 161 | 1.2 | No | 9.2 | 9.2 | 5.5 | N/A | 2.0 | 2.0 |
| T36: 162 | 1.0 | No | 4.5 | 4.5 | 4.5 | N/A | 1.0 | 1.0 |
| T36: 163 | 1.2 | No | 6.5 | 6.5 | 4.5 | N/A | 3.0 | 2.0 |
| T36: 164 | 0.5 | No | 1.5 | 1.5 | N/A | N/A | 3.0 | 2.0 |
| T36: 165 | 1.2 | No | 6.5 | 6.5 | 6.5 | N/A | 2.0 | 2.0 |
| T36: 166 | 1.8 | No | 2.5 | 2.5 | N/A | N/A | 3.0 | 2.0 |
| T36: 167 | 1.8 | No | 8.5 | 8.5 | 8.5 | N/A | 3.0 | 2.0 |
| T36: 168 | 0.2 | No | 4.5 | 4.5 | 0.0 | N/A | 2.0 | 2.0 |
| T36: 169 | 1.2 | No | 6.5 | 6.5 | 4.5 | N/A | 2.0 | 2.0 |
| T36: 170 | 1.8 | No | 5.5 | 5.5 | 4.5 | N/A | 3.0 | 2.0 |
| T36: 171 | 1.5 | No | 8.8 | 8.5 | 4.5 | N/A | 3.0 | 2.0 |
| T36: 172 | 1.5 | No | 6.5 | 6.5 | 5.5 | N/A | 3.0 | 2.0 |
| T36: 173 | 1.2 | No | 4.5 | 4.5 | 3.5 | N/A | 2.0 | 1.0 |
| T36: 174 | 1.8 | No | 7.5 | 7.5 | 5.5 | N/A | 3.0 | 2.0 |
| T36: 175 | 1.8 | No | 8.8 | 8.8 | 5.5 | N/A | 3.0 | 2.0 |
| T36: 176 | 1.0 | No | 6.5 | 6.5 | 4.5 | N/A | 2.0 | 1.0 |
| T36: 177 | 1.5 | No | 8.8 | 8.8 | 6.5 | N/A | 3.0 | 2.0 |
| T36: 178 | 1.0 | No | 2.5 | 2.5 | N/A | N/A | 3.0 | 2.0 |
| T36: 179 | 1.5 | No | 8.5 | 8.5 | 4.5 | N/A | 3.0 | 2.0 |
| T36: 180 | 1.2 | No | 4.5 | 4.5 | 4.5 | N/A | 3.0 | 2.0 |
| T36: 181 | 0.5 | No | 7.5 | 7.5 | 4.5 | N/A | 3.0 | 2.0 |
| T36: 182 | 0.5 | No | 4.5 | 4.5 | 0.0 | N/A | 3.0 | 2.0 |
| T36: 183 | 1.2 | No | 6.5 | 6.5 | 5.5 | N/A | 2.0 | 2.0 |
| T36: 184 | 1.8 | Yes | 7.5 | 7.5 | 4.5 | N/A | 4.0 | 2.0 |
| T36: 185 | 1.2 | No | 5.5 | 5.5 | 3.5 | N/A | 3.0 | 2.0 |
| T36: 186 | 0.5 | No | 5.5 | 5.5 | 3.5 | N/A | 3.0 | 2.0 |
| T36: 187 | 1.0 | No | 5.5 | 5.5 | 4.5 | N/A | 3.0 | 2.0 |
| T36: 188 | 1.0 | No | 6.5 | 6.5 | 4.5 | N/A | 3.0 | 2.0 |
| T36: 189 | 1.2 | No | 6.5 | 6.5 | 4.5 | N/A | 3.0 | 2.0 |
| T36: 190 | 1.2 | No | 7.5 | 7.5 | 6.5 | N/A | 3.0 | 2.0 |
| T36: 191 | 1.5 | No | 6.5 | 6.5 | 6.5 | N/A | 3.0 | 2.0 |
| T36: 192 | 1.0 | No | 8.8 | 9.2 | 7.5 | N/A | 3.0 | 2.0 |
| T36: 193 | 0.5 | No | 8.8 | 9.2 | 7.5 | N/A | 3.0 | 2.0 |
| T36: 194 | 0.5 | No | 5.5 | 5.5 | 4.5 | N/A | 3.0 | 2.0 |
| T36: 195 | 0.5 | No | 8.5 | 8.5 | 8.5 | N/A | 3.0 | 2.0 |
| T36: 196 | 1.0 | Yes | 9.2 | 8.8 | 8.8 | N/A | 4.0 | 2.0 |
| T36: 197 | 1.0 | No | 7.5 | 7.5 | 4.5 | N/A | 4.0 | 2.0 |
| T36: 198 | 1.5 | No | 7.8 | 7.8 | 7.8 | N/A | 3.0 | 1.0 |
| T36: 199 | 1.2 | No | 9.2 | 9.2 | 8.5 | N/A | 4.0 | 3.0 |
| T36: 200 | 3.0 | No | 8.8 | 8.8 | N/A | N/A | 3.0 | 2.0 |
| T36: 201 | 1.8 | No | 7.5 | 8.8 | N/A | N/A | 3.0 | 2.0 |
| T36: 202 | 3.0 | No | 8.8 | 8.8 | N/A | N/A | 3.0 | 2.0 |
| T36: 203 | 1.5 | No | 7.8 | 9.5 | N/A | N/A | 3.0 | 2.0 |
| T36: 204 | 2.0 | No | 7.8 | 9.5 | N/A | N/A | 3.0 | 1.0 |
| T36: 205 | 1.0 | No | 4.8 | 7.8 | N/A | N/A | 3.0 | 1.5 |
| T36: 206 | 1.0 | No | 6.8 | 8.8 | N/A | N/A | 3.0 | 2.0 |
| T36: 207 | 1.0 | No | 6.8 | 9.5 | N/A | N/A | 3.0 | 2.0 |
| T36: 208 | 2.0 | No | 8.8 | 8.8 | N/A | N/A | 3.0 | 3.0 |
| T36: 209 | 5.0 | No | 9.5 | 8.5 | N/A | N/A | 3.0 | 2.0 |
| T36: 210 | 1.5 | No | 9.5 | 8.8 | N/A | N/A | 3.0 | 1.0 |
| T36: 211 | 1.0 | No | 6.8 | 8.8 | N/A | N/A | 3.0 | 2.0 |
| T36: 212 | 4.0 | No | 9.5 | 9.5 | N/A | N/A | 3.0 | 2.0 |
| T36: 213 | 2.0 | No | 7.8 | 8.8 | N/A | N/A | 3.0 | 2.0 |
| T36: 214 | 1.5 | No | 7.8 | 8.8 | N/A | N/A | 3.0 | 1.0 |
| T36: 215 | 1.5 | No | 8.5 | 9.5 | N/A | N/A | 3.0 | 2.0 |
| T36: 216 | 1.8 | No | 8.8 | 8.8 | N/A | N/A | 3.0 | 2.0 |
| T36: 217 | 2.0 | No | 8.8 | 8.8 | N/A | N/A | 3.0 | 3.0 |
| T36: 218 | 4.0 | No | 9.5 | 9.8 | N/A | N/A | 3.0 | 3.0 |
| T36: 219 | 0.5 | No | 3.5 | 8.5 | N/A | N/A | 3.0 | 2.0 |
| T36: 220 | 4.0 | No | 8.8 | 9.5 | N/A | N/A | 3.0 | 2.0 |
| T36: 221 | 0.8 | No | 4.5 | 4.5 | N/A | N/A | 1.0 | 2.0 |
| T36: 222 | 1.0 | No | 4.5 | 4.5 | N/A | N/A | 2.5 | 2.0 |
| T36: 223 | 1.8 | No | 6.5 | 6.5 | N/A | N/A | 3.0 | 2.0 |
| T36: 224 | 1.2 | No | 7.5 | 7.5 | 7.5 | N/A | 3.0 | 2.0 |
| T36: 225 | 1.0 | No | 8.5 | 8.5 | N/A | N/A | 3.0 | 2.0 |
| T36: 226 | 1.5 | No | 8.5 | 8.5 | 8.5 | N/A | 2.5 | 2.0 |
| T36: 227 | 1.8 | No | 8.8 | 9.2 | 9.2 | N/A | 3.0 | 2.0 |
| T36: 228 | 1.8 | No | 8.8 | 9.2 | 9.2 | N/A | 3.0 | 2.0 |
| T36: 229 | 1.0 | No | 8.8 | 9.2 | N/A | N/A | 3.0 | 2.0 |
| T36: 230 | 1.8 | No | 9.2 | 9.2 | N/A | N/A | 3.0 | 2.0 |
| T36: 231 | 1.8 | No | 9.2 | 9.2 | N/A | N/A | 3.5 | 2.0 |
| T36: 232 | 1.8 | No | 9.2 | 9.2 | N/A | N/A | 2.5 | 2.0 |
| T36: 233 | 1.8 | No | 7.5 | 7.5 | N/A | N/A | 3.0 | 2.0 |
| T36: 234 | 1.8 | No | 9.2 | 9.2 | 9.2 | N/A | 2.5 | 1.0 |
| T36: 235 | 1.8 | No | 7.5 | 7.5 | N/A | N/A | 2.5 | 2.0 |
| T36: 236 | 1.8 | No | 8.2 | 8.2 | 8.2 | N/A | 3.5 | 2.0 |
| T36: 237 | 1.8 | No | 9.5 | 9.5 | 5.5 | N/A | 3.0 | 2.0 |
| T36: 238 | 1.8 | No | 7.8 | 7.8 | 4.5 | N/A | 2.5 | 2.0 |
| T36: 239 | 0.5 | No | 8.2 | 8.2 | 5.5 | N/A | 2.0 | 2.0 |
| T36: 240 | 0.8 | No | 6.5 | 6.5 | 6.5 | N/A | 2.0 | 2.0 |
| T36: 241 | 0.8 | No | 8.8 | 9.2 | 7.5 | N/A | 3.0 | 2.0 |
| T36: 242 | 0.5 | No | 8.5 | 8.8 | 4.5 | N/A | 3.5 | 2.0 |
| T36: 243 | 1.8 | No | 4.5 | 4.5 | N/A | N/A | 2.5 | 2.0 |
| T36: 244 | 1.0 | No | 5.5 | 5.5 | 5.5 | N/A | 2.5 | 2.0 |
| T36: 245 | 0.5 | No | 8.8 | 8.8 | 8.8 | N/A | 2.5 | 2.0 |
| T36: 246 | 0.8 | No | 6.5 | 6.5 | 6.5 | N/A | 2.0 | 1.0 |
| T36: 247 | 0.5 | No | 8.5 | 8.5 | 8.5 | N/A | 2.5 | 2.0 |
| T36: 248 | 0.8 | No | 7.5 | 7.5 | 0.0 | N/A | 2.5 | 2.0 |
| T36: 249 | 1.2 | No | 6.5 | 6.5 | 6.5 | N/A | 3.0 | 1.0 |
| T36: 250 | 0.5 | No | 7.5 | 7.5 | 4.5 | N/A | 2.0 | 2.0 |
| T36: 251 | 1.0 | No | 8.8 | 8.8 | 8.8 | N/A | 3.0 | 2.0 |
| T36: 252 | 1.0 | No | 6.5 | 6.5 | 6.5 | N/A | 2.0 | 1.0 |
| T36: 253 | 2.5 | No | 9.2 | 9.2 | 0.0 | N/A | 3.0 | 3.0 |
| T36: 254 | 0.5 | No | 3.5 | 2.5 | 0.0 | N/A | 2.5 | 2.0 |
| T36: 255 | 0.2 | No | 2.5 | 2.5 | N/A | N/A | 2.5 | 2.0 |
| T36: 256 | 1.0 | No | 7.5 | 7.5 | N/A | N/A | 2.5 | 3.0 |
| T36: 257 | 1.5 | No | 8.5 | 9.8 | N/A | N/A | 3.0 | 2.0 |
| T36: 258 | 1.0 | No | 8.5 | 9.5 | N/A | N/A | 3.0 | 2.0 |
| T36: 259 | 1.5 | No | 7.8 | 8.8 | N/A | N/A | 3.0 | 2.0 |
| T36: 260 | 1.0 | No | 8.5 | 7.5 | N/A | N/A | 3.0 | 2.0 |
| T36: 261 | 1.8 | No | 8.5 | 9.5 | N/A | N/A | 3.0 | 2.0 |
| T36: 262 | 1.2 | No | 8.5 | 8.5 | N/A | N/A | 3.0 | 2.0 |
| T36: 263 | 1.2 | No | 4.5 | 1.5 | N/A | N/A | 3.0 | 2.0 |
| T36: 264 | 1.2 | No | 9.5 | 9.8 | N/A | N/A | 3.0 | 2.0 |
| T36: 265 | 1.5 | No | 7.5 | 7.5 | 0.0 | N/A | 3.0 | 2.0 |
| T36: 266 | 1.0 | No | 4.5 | 4.5 | N/A | N/A | 2.5 | 2.0 |
| T36: 267 | 1.2 | No | 7.5 | 7.5 | 3.5 | N/A | 3.0 | 2.0 |
| T36: 268 | 2.0 | Yes | 9.5 | 9.5 | 9.5 | N/A | 4.0 | 3.0 |
| T36: 269 | 1.2 | No | 8.8 | 8.5 | 0.0 | N/A | 2.0 | 2.0 |
| T36: 270 | 1.8 | No | 8.8 | 8.8 | N/A | N/A | 2.5 | 3.0 |
| T36: 271 | 1.8 | Yes | 9.5 | 10.5 | 9.5 | N/A | 3.5 | 3.0 |
| T36: 272 | 1.2 | No | 9.2 | 9.2 | 4.5 | N/A | 1.0 | 2.0 |
| T36: 273 | 1.0 | No | 0.0 | 0.0 | N/A | N/A | 2.0 | 2.0 |
| T36: 274 | 1.2 | No | FO | FO | N/A | N/A | FO | FO |
| T36: 275 | 1.2 | No | 1.5 | 2.5 | N/A | N/A | 2.5 | 2.0 |
| T36: 276 | 0.5 | No | 9.5 | 9.5 | 9.5 | N/A | 3.5 | 2.0 |
| T36: 277 | 2.0 | No | 8.8 | 8.8 | 8.8 | N/A | 1.0 | 2.0 |
| T36: 278 | 1.5 | No | 8.5 | 8.5 | 5.5 | N/A | 3.0 | 3.0 |
| T36: 279 | 0.8 | No | 7.8 | 7.8 | 7.8 | N/A | 2.5 | 1.0 |
| T36: 280 | 0.5 | No | 6.5 | 6.5 | 5.5 | N/A | 4.0 | 2.0 |
| T36: 281 | 0.5 | No | 1.5 | 1.5 | N/A | N/A | 2.0 | 2.0 |
| T36: 282 | 1.0 | No | 7.8 | 7.8 | 5.5 | N/A | 4.0 | 2.0 |

Table 37A shows working examples of measurements of the TSF for the A, B, and C scores after ingestion of one or more treatment substances, wherein the treatment substances were ingested 60 minutes with coffee (Active: caffeine) before the measurements unless otherwise noted herein. Table 37B shows the same working examples with additional sexual function measurements listed. Treatment substances in example T37:41 were ingested with multiple cups of coffee. Treatment substances were ingested with beer in example T37:78. No food, dietary vitamins or nutritional supplements were ingested the day of the measurements until after the measurements were taken, except for about 81 mg up to about 567 mg of aspirin ingested within several hours before the measurements in example T37:13. Measurements were made in the morning from about 7:30 AM until about 11:30 AM.

TABLE 37A
Treatment Substance Ingested Usually with Coffee in the Morning

| | Treatment Substance | | | | | |
|---|---|---|---|---|---|---|
| Example | [Details] | Vendor | mg | C | A | B |
| T37: 1 | MagT | Now/PP/SN | 667 | 1.0 | 6.8 | FE |
| | Theanine | Now | 200 | | | |
| | Inositol | Now | 100 | | | |
| T37: 2 | MagT | Now/PP/SN | 667 | 1.5 | 6.5 | 8.5 |
| | Theanine | Jarrow | 200 | | | |
| T37: 3 | NGABA | AACentral | 200 | 2.5 | 8.5 | 7.5 |
| | MagT | Now/PP/SN | 667 | | | |
| | NAC. SR | Jarrow | 600 | | | |
| | Gly | Now | 1000 | | | |
| | Gln | WFoods | 500 | | | |
| T37: 4 | NGABA | AACentral | 200 | 1.0 | 4.5 | 6.5 |
| | MagT | Now/PP/SN | 667 | | | |
| | GSH | Jarrow | 500 | | | |
| T37: 5 | NGABA | AACentral | 200 | 1.5 | 8.8 | 9.8 |
| | MagT | Now/PP/SN | 2000 | | | |
| | NAC. SR | Jarrow | 600 | | | |
| | Gly | Now | 1000 | | | |
| T37: 6 | NGABA | AACentral | 200 | 1.5 | 8.5 | 8.5 |
| | MagT | Now/PP/SN | 1333 | | | |
| | NAC. SR | Jarrow | 600 | | | |
| | Gly | Now | 1000 | | | |
| | Theanine | Jarrow | 500 | | | |
| T37: 7 | Ile | ON Brand | 750 | 2.0 | 8.5 | 8.8 |
| | Leu | ON Brand | 1500 | | | |
| | Val | ON Brand | 750 | | | |
| T37: 8 | Val | NutraBio | 3150 | 1.0 | 6.5 | 5.5 |
| | MagT | Now/PP/SN | 1333 | | | |
| | NAC. SR | Jarrow | 600 | | | |
| | Gly | Now | 1000 | | | |
| | Theanine | Jarrow | 500 | | | |
| T37: 9 | Ile | ON Brand | 750 | 1.5 | 7.5 | 8.8 |
| | Leu | ON Brand | 1500 | | | |
| | Val | ON Brand | 750 | | | |
| T37: 10 | Val | NutraBio | 3150 | 1.8 | 9.5 | 9.5 |
| | MagT | DBest | 4168 | | | |
| | NAC. SR | Jarrow | 600 | | | |
| | Gly | Now | 1000 | | | |
| | Tyr | WFoods or HEB | 500 | | | |
| T37: 11 | Val | Nutrex | 1250 | 1.8 | 7.5 | 8.5 |
| | Ile | Nutrex | 1250 | | | |
| | Leu | Nutrex | 2500 | | | |
| | Theanine | Jarrow | 200 | | | |
| | NAC. P | WFoods | 500 | | | |
| T37: 12 | Val | Now | 813 | 1.5 | 7.5 | 8.5 |
| | Ile | Now | 813 | | | |
| | Leu | Now | 2000 | | | |
| | NAC. P | WFoods | 500 | | | |
| T37: 13 | Val | NutraBio | 3600 | 2.2 | 8.8 | 9.8 |
| | MagT | DBest | 3126 | | | |
| | NAC. P | WFoods | 500 | | | |
| | Oleamide | Liftmode | 50 | | | |
| T37: 14 | Val | NutraBio | 3600 | 1.0 | 8.5 | 9.5 |
| | MagT | DBest | 3126 | | | |
| | NAC. P | WFoods | 500 | | | |
| | Gly | Now | 2000 | | | |
| | Oleamide | Liftmode | 100 | | | |
| T37: 15 | Val | NutraBio | 3600 | 2.0 | 9.5 | 9.8 |
| | MagT | DBest | 3126 | | | |
| | NAC. P | WFoods | 500 | | | |
| | Oleamide | Liftmode | 100 | | | |
| T37: 16 | Val | NutraBio | 3600 | 1.5 | 9.5 | 8.5 |
| | MagT | DBest | 5210 | | | |
| | NAC. P | WFoods | 500 | | | |
| | Oleamide | Liftmode | 200 | | | |
| T37: 17 | Val | NutraBio | 3600 | 1.8 | 9.5 | 9.5 |
| | MagT | DBest | 5210 | | | |
| | NAC. P | WFoods | 500 | | | |
| T37: 18 | Leu | MHP | 2750 | 0.8 | 0.0 | 2.5 |
| | Ile | MHP | 688 | | | |
| | Val | MHP | 688 | | | |
| | Riboflavin | MHP | 2.1 | | | |
| | Pyridoxine | MHP | 1.9 | | | |
| | MagT | DBest | 5210 | | | |
| T37: 19 | Ile | MHP | 688 | 1.2 | 8.8 | 9.5 |
| | Val | MHP | 688 | | | |
| | Leu | MHP | 2750 | | | |
| | Riboflavin | MHP | 2.1 | | | |
| | Pyridoxine | MHP | 1.9 | | | |
| | MagT | DBest | 3126 | | | |
| | NAC. P | WFoods | 500 | | | |
| T37: 20 | Val | NutraBio | 3600 | 1.5 | 8.8 | 8.8 |
| | MagT | DBest | 3126 | | | |
| | NAC. P | WFoods | 1000 | | | |
| | Gly | Now | 1000 | | | |
| | Oleamide | Liftmode | 100 | | | |
| T37: 21 | Val | NutraBio | 3600 | 1.2 | 8.5 | 8.5 |
| | MagT | DBest | 3126 | | | |
| | NAC. P | WFoods | 1000 | | | |
| | Oleamide | Liftmode | 100 | | | |
| T37: 22 | Val | NutraBio | 3600 | 1.8 | 8.5 | 8.5 |
| | MagT | DBest | 3126 | | | |
| | Gly | Now | 1000 | | | |
| T37: 23 | NGABA | AACentral | 200 | 1.8 | 9.5 | 10.5 |
| | Val | NutraBio | 3600 | | | |
| | MagT | DBest | 3126 | | | |
| | NAC. P* | WFoods | 250 | | | |
| T37: 24 | NGABA | AACentral | 200 | 2.2 | 9.5 | 9.8 |
| | Val | NutraBio | 3600 | | | |
| | MagT | DBest | 3126 | | | |
| | NAC. P | WFoods | 1000 | | | |
| T37: 25 | Val | NutraBio | 3600 | 1.8 | 9.5 | 8.5 |
| | MagT | DBest | 3126 | | | |
| | NAC. P | WFoods | 2000 | | | |
| T37: 26 | Val | NutraBio | 3600 | 1.8 | 9.5 | 9.5 |
| | NAC. P | WFoods | 500 | | | |
| | Cys | SwansonF1 | 500 | | | |
| T37: 27 | Val | NutraBio | 3600 | 1.8 | 9.5 | 9.5 |
| | MagT | DBest | 3126 | | | |
| | NAC. P | WFoods | 500 | | | |
| | Cys | SwansonF1 | 500 | | | |
| T37: 28 | Val | NutraBio | 3600 | 1.8 | 8.5 | 8.5 |
| | NAC. P | WFoods | 500 | | | |
| | Gln | WFoods | 500 | | | |
| T37: 29 | Val | NutraBio | 3600 | 1.8 | 8.5 | 9.5 |
| | NAC. P | WFoods | 500 | | | |
| | 5HTP | Bluebonnet | 100 | | | |
| T37: 30 | Val | NutraBio | 3600 | 1.8 | 9.5 | 9.5 |
| | MagT | DBest | 3126 | | | |
| | NAC. P | WFoods | 500 | | | |
| | Gly | Now | 1000 | | | |
| | AKG | Klaire Labs | 300 | | | |
| T37: 31 | NGABA | AACentral | 200 | 1.5 | FO | FO |
| | NAC. P | WFoods | 500 | | | |
| | Ketoisocaproate | Twinlab | 500 | | | |
| T37: 32 | Val | NutraBio | 3600 | 2.0 | 9.8 | 9.5 |
| | MagT | DBest | 3126 | | | |
| | NAC. P | WFoods | 500 | | | |
| | Gly | Now | 1000 | | | |
| | Acetyl-Carnitine HCl | Jarrow | 500 | | | |
| T37: 33 | Val | NutraBio | 3600 | 1.8 | 8.8 | 9.5 |
| | MagT | DBest | 3126 | | | |
| | NAC. P | WFoods | 500 | | | |
| | Tocopheryl Succinate | Now | 658 | | | |
| T37: 34 | Val | NutraBio | 3600 | 2.0 | 9.5 | 9.5 |
| | MagT | DBest | 3126 | | | |
| | NAC. P | WFoods | 500 | | | |
| | Theanine | Now | 600 | | | |
| | Inositol | Now | 300 | | | |
| T37: 35 | NGABA | AACentral | 200 | 0.5 | 8.8 | 8.8 |
| | Tyr | WFoods or HEB | 1000 | | | |
| | MagT | DBest | 3126 | | | |
| | NAC. P | WFoods | 500 | | | |
| | Caffeine* | Nutrakey | 100 | | | |
| T37: 36 | Val | NutraBio | 3600 | 1.8 | 9.5 | 9.8 |
| | MagT | DBest | 3126 | | | |
| | NAC. P | WFoods | 500 | | | |
| | D-Ser | Relentless | 700 | | | |
| | DimethylGly | Now | 125 | | | |
| T37: 37 | Val | NutraBio | 3600 | 2.0 | 9.5 | 9.5 |
| | MagT | DBest | 3126 | | | |
| | NAC. P | WFoods | 500 | | | |
| | Galantamine HBr | Relentless | 4 | | | |
| | [1 Tablet CW**] | | | | | |
| T37: 38 | Val | NutraBio | 3600 | 2.5 | 9.8 | 9.5 |
| | MagT | DBest | 3126 | | | |
| | NAC. P | WFoods | 500 | | | |
| | Alpha- | SwansonHP | 300 | | | |
| | GlyceroPhosphoCholine | | | | | |
| | Galantamine HBr | Relentless | 4 | | | |
| | [1 Tablet CW**] | | | | | |
| T37: 39 | NGABA | AACentral | 200 | 1.8 | 9.5 | 9.5 |
| | MagT | DBest | 3126 | | | |
| | NAC. P | WFoods | 500 | | | |
| | Galantamine HBr | Relentless | 8 | | | |
| | [2 Tablets CW**] | | | | | |
| T37: 40 | Val | NutraBio | 3600 | 1.8 | 9.8 | 9.5 |
| | NAC. SR | Jarrow | 600 | | | |
| | NAC. P | WFoods | 500 | | | |
| | Galantamine HBr | Relentless | 12 | | | |
| T37: 41 | Val | NutraBio | 3600 | 1.8 | 9.8 | 9.8 |
| | NAC. SR | Jarrow | 600 | | | |
| | NAC. P | WFoods | 500 | | | |
| | Galantamine HBr | Relentless | 12 | | | |
| | MagT | DBest | 3126 | | | |
| T37: 42 | NGABA | AACentral | 200 | 1.8 | 8.8 | 8.8 |
| | MagT | DBest | 3126 | | | |
| | NAC. P | WFoods | 500 | | | |
| | D-Ser | Relentless | 700 | | | |
| | DimethylGly | Now | 125 | | | |
| | Galantamine HBr | Relentless | 4 | | | |
| T37: 43 | NGABA | AACentral | 300 | 1.8 | 8.8 | 8.5 |
| | MagT | DBest | 3126 | | | |
| | NAC. P | WFoods | 500 | | | |
| | D-Ser | Relentless | 700 | | | |
| | DimethylGly | Now | 125 | | | |
| | Galantamine HBr | Relentless | 4 | | | |
| | 5HTP | Bluebonnet | 100 | | | |
| T37: 44 | NGABA | AACentral | 200 | 1.2 | 8.8 | 8.5 |
| | NAC. P | WFoods | 500 | | | |
| | Alpha- | SwansonHP | 300 | | | |
| | GlyceroPhosphoCholine | | | | | |
| | DimethylGly | Now | 125 | | | |
| | Galantamine HBr | Relentless | 4 | | | |
| T37: 45 | Val | NutraBio | 3600 | 2.0 | 9.2 | 9.2 |
| | MagT | DBest | 3126 | | | |
| | NAC. P | WFoods | 500 | | | |
| | DimethylGly | Now | 125 | | | |
| | Galantamine HBr | Relentless | 4 | | | |
| | Huperzine A | SwansonHP | 0.05 | | | |
| T37: 46 | Val | NutraBio | 3600 | 1.2 | 9.5 | 9.5 |
| | MagT | DBest | 3126 | | | |
| | NAC. P | WFoods | 500 | | | |
| | DimethylGly | Now | 125 | | | |
| | Galantamine HBr | Relentless | 4 | | | |
| | Huperzine A | SwansonHP | 0.05 | | | |
| | D-Ser | Relentless | 700 | | | |
| | 50% Trans-Resveratrol | WFoods | 500 | | | |
| T37: 47 | NGABA | AACentral | 200 | 1.0 | 9.2 | 9.2 |
| | Leu | SNaturals | 4000 | | | |
| | NAC. P | WFoods | 500 | | | |
| | MagT | DBest | 3126 | | | |
| | Galantamine HBr | Relentless | 4 | | | |
| | Huperzine A | SwansonHP | 0.05 | | | |
| | Inosine | SwansonHP | 500 | | | |
| T37: 48 | Val | BulkSupps | 3600 | 1.2 | 9.5 | 9.5 |
| | MagT | DBest | 3126 | | | |
| | NAC. P | WFoods | 500 | | | |
| | DimethylGly | Now | 125 | | | |
| | Galantamine HBr | Relentless | 4 | | | |
| | D-Ser | Relentless | 700 | | | |
| | Ubiquinone | NaturesLab | 100 | | | |
| | Alpha-Lipoic Acid | NaturesLab | 150 | | | |
| | Acetyl-Carnitine | NaturesLab | 125 | | | |
| T37: 49 | Val | BulkSupps | 3600 | 2.2 | 9.2 | 9.2 |
| | MagT | DBest | 3126 | | | |
| | NAC. P | WFoods | 500 | | | |
| | Galantamine HBr | Relentless | 4 | | | |
| | D-Ser | Relentless | 700 | | | |
| | Inosine | SwansonHP | 500 | | | |
| T37: 50 | Val | BulkSupps | 3600 | 2.0 | 9.5 | 9.5 |
| | MagT | DBest | 3126 | | | |
| | NAC. P | WFoods | 500 | | | |
| | Centrophenoxine | HealthN | 500 | | | |
| T37: 51 | Val | BulkSupps | 3600 | 1.5 | 9.2 | 9.2 |
| | MagT | DBest | 3126 | | | |
| | NAC. P | WFoods | 500 | | | |
| | 95% Baicalin | Liftmode | 270 | | | |
| T37: 52 | Val | BulkSupps | 3600 | 1.8 | 9.2 | 9.2 |
| | MagT | DBest | 3126 | | | |
| | NAC. P | WFoods | 500 | | | |
| | Inosine | SwansonHP | 1500 | | | |
| | NGABA | AACentral | 200 | | | |
| | 95% Baicalin | Liftmode | 810 | | | |
| T37: 53 | Homotaurine | Supersmart | 100 | 2.0 | 9.5 | 8.5 |
| | DimethylGly | Now | 125 | | | |
| T37: 54 | Homotaurine | Supersmart | 100 | 1.8 | 9.5 | 9.5 |
| | D-Ser | Relentless | 700 | | | |
| T37: 55 | Betaine | BulkSupps | 650 | 0.5 | 0.0 | 2.5 |
| T37: 56 | Homotaurine | Supersmart | 250 | 1.2 | 7.5 | 7.5 |
| T37: 57 | Homotaurine | Supersmart | 200 | 1.2 | 9.2 | 9.2 |
| T37: 58 | Val | BulkSupps | 4320 | 1.5 | 9.5 | 9.5 |
| | NAC. P | WFoods | 500 | | | |
| | Norvaline | BulkSupps | 1084 | | | |
| T37: 59 | Val | BulkSupps | 4320 | 1.5 | 9.2 | 9.2 |
| | NAC. P | WFoods | 500 | | | |
| | MagT | DBest | 3126 | | | |
| | DimethylGly | Now | 125 | | | |
| | Sarcosine | Brainvitamiz | 620 | | | |
| | Betaine | BulkSupps | 650 | | | |
| T37: 60 | Val | BulkSupps | 4320 | 1.5 | 9.5 | 9.5 |
| | NAC. P | WFoods | 500 | | | |
| | MagT | DBest | 3126 | | | |
| | Norvaline | BulkSupps | 2168 | | | |
| T37: 61 | Val | BulkSupps | 4320 | 1.2 | 9.2 | 9.2 |
| | NAC. P | WFoods | 500 | | | |
| | Gly | Now | 1000 | | | |
| T37: 62 | Leu | SNaturals | 4000 | 1.2 | 9.2 | 9.2 |
| | NAC. P | WFoods | 500 | | | |
| | MagT | DBest | 3126 | | | |
| | Inosine | SwansonHP | 500 | | | |
| T37: 63 | Val | BulkSupps | 5040 | 1.0 | 9.5 | 9.5 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 290 | | | |
| | Calcium Pyruvate | BulkSupps | 899 | | | |
| T37: 64 | Val | BulkSupps | 5040 | 1.5 | 8.5 | 7.5 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 290 | | | |
| | Calcium Pyruvate | BulkSupps | 899 | | | |
| | Panax ginseng | BulkSupps | 600 | | | |
| T37: 65 | Val | NutraBio | 3150 | 1.5 | 7.5 | 9.5 |
| | NGABA | AACentral | 100 | | | |
| | MagT | Now/PP/SN | 667 | | | |
| T37: 66 | Val | NutraBio | 3150 | 1.5 | 7.8 | 9.8 |
| | MagT | Now/PP/SN | 667 | | | |
| | Cat's Claw Bark | NaturesWay | 160 | | | |
| | Cat's Claw | NaturesWay | 175 | | | |
| | Bark Extract | | | | | |
| T37: 67 | Val | NutraBio | 3150 | 4.0 | 9.5 | 9.5 |
| | NAC. P | WFoods | 1000 | | | |
| T37: 68 | Val | NutraBio | 3150 | 1.5 | 9.5 | 9.5 |
| | NAC. P | WFoods | 1000 | | | |
| | MagT | Now/PP/SN | 667 | | | |
| T37: 69 | NGABA | AACentral | 200 | 5.0 | 9.8 | 9.8 |
| | NAC. P | WFoods | 500 | | | |
| | Gly | Now | 1000 | | | |
| | MagT | Now/PP/SN | 667 | | | |
| | 5HTP | Bluebonnet | 100 | | | |
| T37: 70 | Ile | ON Brand | 750 | 2.0 | 8.8 | 8.8 |
| | Val | ON Brand | 750 | | | |
| | Leu | ON Brand | 1500 | | | |
| | Gln | WFoods | 1000 | | | |
| | MagT | Now/PP/SN | 667 | | | |
| T37: 71 | Val | NutraBio | 3150 | 1.8 | 9.5 | 9.5 |
| | MagT | Now/PP/SN | 667 | | | |
| | NAC. P | WFoods | 500 | | | |
| | Gly | Now | 1000 | | | |
| T37: 72 | Val | NutraBio | 3150 | 2.0 | 8.5 | 9.5 |
| | NAC. P | WFoods | 500 | | | |
| | MagT | Now/PP/SN | 667 | | | |
| | Boswellia | Himalaya | 250 | | | |
| T37: 73 | NGABA | AACentral | 200 | 1.8 | 8.5 | 9.5 |
| | NAC. P | WFoods | 500 | | | |
| | MagT | Now/PP/SN | 667 | | | |
| | Boswellia | Himalaya | 250 | | | |
| T37: 74 | Val | BulkSupps | 4320 | 1.8 | 9.2 | 9.2 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | Caffeine | Nutrakey | 200 | | | |
| T37: 75 | NGABA | AACentral | 300 | 1.8 | 9.5 | 9.5 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 580 | | | |
| | Beta-Ala | Now | 1500 | | | |
| T37: 76 | NGABA | AACentral | 300 | 2.0 | 9.5 | 9.5 |
| | MagT | Now/PP/SN | 2000 | | | |
| | NAC. P | BulkSupps | 720 | | | |
| | Huperzine A | SwansonHP | 0.1 | | | |
| | Saffron Extract | PipingRock | 177 | | | |
| | Lemon Balm | OWH | 750 | | | |
| | Caffeine | Nutrakey | 200 | | | |
| T37: 77 | Val | BulkSupps | 4320 | 1.0 | 8.8 | 9.2 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 1280 | | | |
| | Beta-Ala | Now | 1500 | | | |
| T37: 78 | Val | BulkSupps | 4320 | 0.8 | 8.8 | 8.8 |
| | MagT | Now/PP/SN | 2000 | | | |
| | NAC. P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Olive | Synergy | 1400 | | | |
| T37: 79 | Val | BulkSupps | 4320 | 0.8 | 8.8 | 8.5 |
| | NAC. P | BulkSupps | 720 | | | |
| | MagT | Now/PP/SN | 2000 | | | |
| | Saffron Extract | PipingRock | 177 | | | |
| | Huperzine A | SwansonHP | 0.1 | | | |
| | Kava | BulkSupps | 640 | | | |
| | Sodium Butyrate | Bodybio | 3027 | | | |
| T37: 80 | Pramiracetam | NoopDepot | 625 | 0.2 | 8.8 | 8.8 |
| T37: 81 | Val | BulkSupps | 5760 | 0.5 | 9.2 | 9.2 |
| | NAC. P | BulkSupps | 720 | | | |
| | Pramiracetam | NoopDepot | 625 | | | |
| T37: 82 | Val | BulkSupps | 5760 | 0.2 | 9.2 | 8.8 |
| | NAC. P | BulkSupps | 720 | | | |
| | 98% Icariin | Liftmode | 60 | | | |
| T37: 83 | Val | BulkSupps | 5760 | 1.2 | 8.8 | 8.8 |
| | NAC. P | BulkSupps | 720 | | | |
| | MagT | Now/PP/SN | 2000 | | | |
| | Phenibut | Liftmode | 601 | | | |
| | Saffron Extract | PipingRock | 177 | | | |
| T37: 84 | Val | BulkSupps | 5040 | 0.5 | 9.2 | 9.2 |
| | NAC.P | BulkSupps | 720 | | | |
| | Pramiracetam | NoopDepot | 250 | | | |
| T37: 85 | Val | BulkSupps | 4320 | 1.8 | 9.5 | 9.5 |
| | NAC. P | BulkSupps | 720 | | | |
| | Saffron Extract | PipingRock | 177 | | | |
| | Huperzine A | SwansonHP | 0.1 | | | |
| | Kava | BulkSupps | 640 | | | |
| | Valerian | WFoods | 1000 | | | |
| T37: 86 | Val | BulkSupps | 4320 | 1.0 | 9.2 | 9.2 |
| | NAC. P | BulkSupps | 720 | | | |
| | MagT | Now/PP/SN | 1333 | | | |
| | Saffron Extract | PipingRock | 177 | | | |
| | Huperzine A | SwansonHP | 0.1 | | | |
| | Kava | BulkSupps | 640 | | | |
| | Cinchona | Terra Vita | 400 | | | |
| T37: 87 | Val | BulkSupps | 3600 | 1.2 | 8.5 | 8.5 |
| | MagT | DBest | 3126 | | | |
| | NGABA | AACentral | 200 | | | |
| | NAC. P | WFoods | 500 | | | |
| | Lemon Balm | OWH | 375 | | | |
| | Beta-Ala | Now | 750 | | | |
| T37: 88 | Leu | Nutrex | 2500 | 1.0 | 9.5 | 9.5 |
| | Ile | Nutrex | 1250 | | | |
| | Val | Nutrex | 1250 | | | |
| | MagT | DBest | 5210 | | | |

*About half of the powder removed from capsule;
**“Tablet(s) CW” means the tablet(s) were chewed before swallowing to promote rapid absorption

TABLE 37B
Treatment Substance Ingested Usually with Coffee in the Morning

| Example | C | CT | A | B | BM | BS | D | DE |
|---|---|---|---|---|---|---|---|---|
| T37: 1 | 1.0 | No | 6.8 | FE | N/A | N/A | FE | FE |
| T37: 2 | 1.5 | No | 6.5 | 8.5 | N/A | N/A | 3.0 | 1.0 |
| T37: 3 | 2.5 | No | 8.5 | 7.5 | N/A | N/A | 3.0 | 2.0 |
| T37: 4 | 1.0 | No | 4.5 | 6.5 | N/A | N/A | 3.0 | 2.0 |
| T37: 5 | 1.5 | No | 8.8 | 9.8 | N/A | N/A | 3.0 | 2.0 |
| T37: 6 | 1.5 | No | 8.5 | 8.5 | N/A | N/A | 3.0 | 2.0 |
| T37: 7 | 2.0 | No | 8.5 | 8.8 | N/A | N/A | 3.0 | 2.0 |
| T37: 8 | 1.0 | No | 6.5 | 5.5 | N/A | N/A | 3.0 | 2.0 |
| T37: 9 | 1.5 | No | 7.5 | 8.8 | N/A | N/A | 3.0 | 2.0 |
| T37: 10 | 1.8 | No | 9.5 | 9.5 | N/A | N/A | 3.0 | 3.0 |
| T37: 11 | 1.8 | No | 7.5 | 8.5 | N/A | N/A | 3.0 | 2.0 |
| T37: 12 | 1.5 | No | 7.5 | 8.5 | N/A | N/A | 3.0 | 2.0 |
| T37: 13 | 2.2 | No | 8.8 | 9.8 | N/A | N/A | 3.0 | 2.0 |
| T37: 14 | 1.0 | No | 8.5 | 9.5 | N/A | N/A | 3.0 | 2.0 |
| T37: 15 | 2.0 | No | 9.5 | 9.8 | N/A | N/A | 4.0 | 2.0 |
| T37: 16 | 1.5 | No | 9.5 | 8.5 | 0.0 | N/A | 3.0 | 2.0 |
| T37: 17 | 1.8 | No | 9.5 | 9.5 | 7.5 | N/A | 3.0 | 2.0 |
| T37: 18 | 0.8 | No | 0.0 | 2.5 | N/A | N/A | 3.0 | 2.0 |
| T37: 19 | 1.2 | No | 8.8 | 9.5 | N/A | N/A | 3.0 | 2.0 |
| T37: 20 | 1.5 | No | 8.8 | 8.8 | 0.0 | N/A | 3.0 | 2.0 |
| T37: 21 | 1.2 | No | 8.5 | 8.5 | 0.0 | N/A | 3.0 | 2.0 |
| T37: 22 | 1.8 | No | 8.5 | 8.5 | 0.0 | N/A | 3.0 | 2.0 |
| T37: 23 | 1.8 | No | 9.5 | 10.5 | 3.5 | N/A | 3.0 | 2.0 |
| T37: 24 | 2.2 | No | 9.5 | 9.8 | 0.0 | N/A | 3.0 | 2.0 |
| T37: 25 | 1.8 | No | 9.5 | 8.5 | 0.0 | N/A | 3.0 | 2.0 |
| T37: 26 | 1.8 | No | 9.5 | 9.5 | 0.0 | N/A | 4.0 | 2.0 |
| T37: 27 | 1.8 | No | 9.5 | 9.5 | 0.0 | N/A | 4.0 | 2.0 |
| T37: 28 | 1.8 | No | 8.5 | 8.5 | 0.0 | N/A | 3.0 | 2.0 |
| T37: 29 | 1.8 | No | 8.5 | 9.5 | N/A | N/A | 3.0 | 2.0 |
| T37: 30 | 1.8 | No | 9.5 | 9.5 | 0.0 | N/A | 5.0 | 2.0 |
| T37: 31 | 1.5 | No | FO | FO | N/A | N/A | FO | FO |
| T37: 32 | 2.0 | No | 9.8 | 9.5 | 0.0 | N/A | 4.0 | 2.0 |
| T37: 33 | 1.8 | No | 8.8 | 9.5 | 4.5 | N/A | 3.5 | 2.0 |
| T37: 34 | 2.0 | No | 9.5 | 9.5 | 0.0 | N/A | 4.0 | 2.0 |
| T37: 35 | 0.5 | No | 8.8 | 8.8 | 6.5 | N/A | 4.0 | 2.0 |
| T37: 36 | 1.8 | No | 9.5 | 9.8 | N/A | N/A | 4.0 | 2.0 |
| T37: 37 | 2.0 | No | 9.5 | 9.5 | 5.5 | N/A | 4.0 | 2.0 |
| T37: 38 | 2.5 | No | 9.8 | 9.5 | 0.0 | N/A | 5.0 | 2.0 |
| T37: 39 | 1.8 | Yes | 9.5 | 9.5 | N/A | N/A | 4.0 | 3.0 |
| T37: 40 | 1.8 | Yes | 9.8 | 9.5 | 4.5 | N/A | 4.0 | 2.0 |
| T37: 41 | 1.8 | No | 9.8 | 9.8 | 0.0 | N/A | 4.0 | 2.0 |
| T37: 42 | 1.8 | Yes | 8.8 | 8.8 | 7.5 | N/A | 4.0 | 3.0 |
| T37: 43 | 1.8 | No | 8.8 | 8.5 | 6.5 | N/A | 4.0 | 3.0 |
| T37: 44 | 1.2 | No | 8.8 | 8.5 | 2.5 | N/A | 4.0 | 2.0 |
| T37: 45 | 2.0 | No | 9.2 | 9.2 | 9.2 | N/A | 4.0 | 2.0 |
| T37: 46 | 1.2 | Yes | 9.5 | 9.5 | 9.5 | N/A | 5.0 | 3.0 |
| T37: 47 | 1.0 | No | 9.2 | 9.2 | 9.2 | N/A | 4.0 | 2.0 |
| T37: 48 | 1.2 | No | 9.5 | 9.5 | 0.0 | N/A | 4.0 | 2.0 |
| T37: 49 | 2.2 | No | 9.2 | 9.2 | 9.2 | N/A | 4.0 | 2.0 |
| T37: 50 | 2.0 | No | 9.5 | 9.5 | 0.0 | FT | 4.0 | 2.0 |
| T37: 51 | 1.5 | No | 9.2 | 9.2 | N/A | N/A | 4.0 | 2.0 |
| T37: 52 | 1.8 | No | 9.2 | 9.2 | 2.5 | SL | 4.0 | 2.0 |
| T37: 53 | 2.0 | Yes | 9.5 | 8.5 | 7.5 | N/A | 4.0 | 2.0 |
| T37: 54 | 1.8 | No | 9.5 | 9.5 | 0.0 | N/A | 4.0 | 2.0 |
| T37: 55 | 0.5 | No | 0.0 | 2.5 | N/A | N/A | 3.0 | 2.0 |
| T37: 56 | 1.2 | No | 7.5 | 7.5 | 6.5 | N/A | 4.0 | 2.0 |
| T37: 57 | 1.2 | No | 9.2 | 9.2 | 5.5 | N/A | 4.0 | 2.0 |
| T37: 58 | 1.5 | No | 9.5 | 9.5 | 7.5 | N/A | 4.0 | 2.0 |
| T37: 59 | 1.5 | No | 9.2 | 9.2 | 7.5 | N/A | 4.0 | 2.0 |
| T37: 60 | 1.5 | No | 9.5 | 9.5 | 6.5 | N/A | 4.0 | 2.0 |
| T37: 61 | 1.2 | No | 9.2 | 9.2 | 8.5 | N/A | 4.0 | 2.0 |
| T37: 62 | 1.2 | No | 9.2 | 9.2 | 9.2 | N/A | 4.0 | 2.0 |
| T37: 63 | 1.0 | No | 9.5 | 9.5 | 7.5 | N/A | 4.0 | 2.0 |
| T37: 64 | 1.5 | No | 8.5 | 7.5 | 4.5 | N/A | 4.0 | 2.0 |
| T37: 65 | 1.5 | No | 7.5 | 9.5 | N/A | N/A | 3.0 | 2.0 |
| T37: 66 | 1.5 | No | 7.8 | 9.8 | N/A | N/A | 3.0 | 2.0 |
| T37: 67 | 4.0 | No | 9.5 | 9.5 | N/A | N/A | 3.0 | 2.0 |
| T37: 68 | 1.5 | No | 9.5 | 9.5 | N/A | N/A | 3.0 | 2.0 |
| T37: 69 | 5.0 | No | 9.8 | 9.8 | N/A | N/A | 3.0 | 2.0 |
| T37: 70 | 2.0 | No | 8.8 | 8.8 | N/A | N/A | 3.0 | 2.0 |
| T37: 71 | 1.8 | No | 9.5 | 9.5 | N/A | N/A | 3.0 | 2.0 |
| T37: 72 | 2.0 | No | 8.5 | 9.5 | N/A | N/A | 3.0 | 3.0 |
| T37: 73 | 1.8 | No | 8.5 | 9.5 | N/A | N/A | 3.0 | 3.0 |
| T37: 74 | 1.8 | No | 9.2 | 9.2 | 6.5 | N/A | 4.0 | 2.0 |
| T37: 75 | 1.8 | No | 9.5 | 9.5 | 6.5 | N/A | 4.0 | 2.0 |
| T37: 76 | 2.0 | No | 9.5 | 9.5 | 8.5 | N/A | 4.0 | 2.0 |
| T37: 77 | 1.0 | Yes | 8.8 | 9.2 | 7.5 | N/A | 4.0 | 2.0 |
| T37: 78 | 0.8 | No | 8.8 | 8.8 | 8.8 | N/A | 2.5 | 2.0 |
| T37: 79 | 0.8 | No | 8.8 | 8.5 | 6.5 | N/A | 3.5 | 2.0 |
| T37: 80 | 0.2 | No | 8.8 | 8.8 | 8.8 | N/A | 3.0 | 2.0 |
| T37: 81 | 0.5 | No | 9.2 | 9.2 | 0.0 | FT | 4.0 | 3.0 |
| T37: 82 | 0.2 | No | 9.2 | 8.8 | 8.8 | N/A | 4.0 | 2.0 |
| T37: 83 | 1.2 | No | 8.8 | 8.8 | 0.0 | FT | 4.0 | 2.0 |
| T37: 84 | 0.5 | No | 9.2 | 9.2 | 9.2 | FT | 4.0 | 2.0 |
| T37: 85 | 1.8 | No | 9.5 | 9.5 | 6.5 | FT | 4.0 | 2.0 |
| T37: 86 | 1.0 | No | 9.2 | 9.2 | 6.5 | N/A | 3.5 | 2.0 |
| T37: 87 | 1.2 | No | 8.5 | 8.5 | N/A | N/A | 4.0 | 2.0 |
| T37: 88 | 1.0 | No | 9.5 | 9.5 | N/A | N/A | 3.0 | 2.0 |

Table 38A shows working examples of measurements of the TSF for the A, B, and C scores after ingestion of one or more treatment substances, wherein the treatment substances were ingested 60 minutes with wine and/or beer (Active: ethanol; “Alcohol”) before the measurements. Table 38B shows the same working examples with additional sexual function measurements listed. Dietary vitamins and nutritional supplements were ingested about 7:00 AM to about 10:30 AM with coffee, and coffee consumption ended by about 12:00 PM or earlier. Food intake was not recorded, but was likely ingested an hour or more treatment substance ingestion in examples T38:3, T38:4, T38:6, T38:9, T38:9, T38:11, T38:12, T38:13, T38:14, T38:15, T38:16, and T38:17. All measurements were made in the afternoon to late evening from about 4:55 PM until about 10 PM.

TABLE 38A
Treatment Substance Ingested with
Alcohol in the Afternoon/Evening

| | Treatment Substance | | | | | |
|---|---|---|---|---|---|---|
| Example | [Details] | Vendor | mg | C | A | B |
| T38: 1 | Val | NutraBio | 3150 | 2.5 | 9.5 | 9.5 |
| | MagT | Now/PP/SN | 1333 | | | |
| | NAC. SR | Jarrow | 600 | | | |
| | Gly | Now | 1000 | | | |
| T38: 2 | Val | BulkSupps | 3600 | 2.2 | 9.5 | 9.2 |
| | MagT | DBest | 3126 | | | |
| | NAC. P | WFoods | 500 | | | |
| | Galantamine HBr | Relentless | 4 | | | |
| | D-Ser | Relentless | 700 | | | |
| | Inosine | SwansonHP | 1500 | | | |
| T38: 3 | NGABA | AACentral | 200 | 2.0 | 9.5 | 9.5 |
| | Homotaurine | Supersmart | 50 | | | |
| T38: 4 | Val | BulkSupps | 3600 | 1.2 | 7.5 | 7.5 |
| | Homotaurine | Supersmart | 100 | | | |
| T38: 5 | Homotaurine | Supersmart | 150 | 1.8 | 9.5 | 9.5 |
| T38: 6 | Sarcosine | Brainvitamiz | 620 | 1.2 | 6.5 | 6.5 |
| T38: 7 | Val | BulkSupps | 3600 | 1.5 | 6.5 | 5.5 |
| | NAC. P | WFoods | 500 | | | |
| | Sarcosine | Brainvitamiz | 620 | | | |
| T38: 8 | Val | BulkSupps | 4320 | 1.5 | 9.2 | 9.2 |
| | NAC. P | WFoods | 500 | | | |
| T38: 9 | Val | BulkSupps | 4320 | 1.2 | 7.5 | 7.5 |
| | NAC. P | WFoods | 500 | | | |
| | AcetylGln | BulkSupps | 666 | | | |
| | MagT | DBest | 3126 | | | |
| T38: 10 | Val | BulkSupps | 4320 | 1.2 | 9.5 | 9.5 |
| | NAC. P | WFoods | 500 | | | |
| | Norvaline | BulkSupps | 263 | | | |
| T38: 11 | Val | BulkSupps | 4320 | 1.5 | 8.8 | 8.8 |
| | NAC. P | WFoods | 500 | | | |
| | MagT | DBest | 3126 | | | |
| | D-Ser | Relentless | 7000 | | | |
| T38: 12 | Val | NutraBio | 3150 | 3.0 | 8.5 | 9.5 |
| | Gly | Now | 1000 | | | |
| | MagT | Now/PP/SN | 667 | | | |
| | Taurine | Now | 1000 | | | |
| T38: 13 | Val | NutraBio | 3150 | 2.0 | 7.8 | 9.8 |
| | MagT | Now/PP/SN | 667 | | | |
| | NAC. P | WFoods | 500 | | | |
| | Propolis Extract | Y.S. Farms | 1000 | | | |
| T38: 14 | Collagen Peptides | Vital Proteins | 736 | 2.2 | 9.2 | 9.2 |
| | NGABA | LifeLink | 200 | | | |
| T38: 15 | Theanine | SwansonHP | 500 | 0.8 | 5.5 | 5.5 |
| T38: 16 | Phenylethylamine | Liftmode | 624 | 0.2 | 5.5 | 5.5 |
| T38: 17 | Pramiracetam | NoopDepot | 125 | 0.5 | 6.5 | 6.5 |
| T38: 18 | Homotaurine | Supersmart | 50 | 1.0 | 9.2 | 9.2 |

TABLE 38B
Treatment Substance Ingested with
Alcohol in the Afternoon/Evening

| Example | C | CT | A | B | BM | BS | D | DE |
|---|---|---|---|---|---|---|---|---|
| T38: 1 | 2.5 | No | 9.5 | 9.5 | N/A | N/A | 4.0 | 2.0 |
| T38: 2 | 2.2 | No | 9.5 | 9.2 | N/A | N/A | 4.0 | 2.0 |
| T38: 3 | 2.0 | No | 9.5 | 9.5 | 7.5 | N/A | 3.0 | 2.0 |
| T38: 4 | 1.2 | No | 7.5 | 7.5 | 4.5 | N/A | 3.0 | 2.0 |
| T38: 5 | 1.8 | No | 9.5 | 9.5 | 7.5 | N/A | 2.5 | 3.0 |
| T38: 6 | 1.2 | No | 6.5 | 6.5 | 4.5 | N/A | 3.0 | 2.0 |
| T38: 7 | 1.5 | No | 6.5 | 5.5 | N/A | N/A | 3.0 | 2.0 |
| T38: 8 | 1.5 | No | 9.2 | 9.2 | 4.5 | N/A | 3.0 | 2.0 |
| T38: 9 | 1.2 | No | 7.5 | 7.5 | 3.5 | N/A | 2.0 | 1.0 |
| T38: 10 | 1.2 | No | 9.5 | 9.5 | 4.5 | N/A | 3.0 | 2.0 |
| T38: 11 | 1.5 | No | 8.8 | 8.8 | 6.5 | N/A | 3.0 | 2.0 |
| T38: 12 | 3.0 | No | 8.5 | 9.5 | N/A | N/A | 3.0 | 2.0 |
| T38: 13 | 2.0 | No | 7.8 | 9.8 | N/A | N/A | 3.0 | 2.0 |
| T38: 14 | 2.2 | No | 9.2 | 9.2 | 0.0 | N/A | 3.0 | 3.0 |
| T38: 15 | 0.8 | No | 5.5 | 5.5 | 5.5 | N/A | 3.0 | 2.0 |
| T38: 16 | 0.2 | No | 5.5 | 5.5 | 5.5 | N/A | 3.0 | 2.0 |
| T38: 17 | 0.5 | No | 6.5 | 6.5 | 0.0 | IS | 3.0 | 2.0 |
| T38: 18 | 1.0 | No | 9.2 | 9.2 | 0.0 | N/A | 3.0 | 2.0 |

Table 39A shows working examples of measurements of sexual function scores after ingestion of one or more treatment substances with various beverages, wherein the treatment substances were ingested at times other than 60 minutes before the measurements. Table 39B and Table 39C show the same working examples with measurements of the TSF for the A, B, and C scores with additional sexual function measurements shown at Table 39C. Examples measured in the morning, afternoon, and evening had the same dietary vitamins, nutritional supplements and food ingested as described for Table 37A, Table 36A, and Table 38A, respectively, though food likely was ingested within a few hours before each example in the evening. Examples measured in the morning, afternoon, and evening were between about 8:05 AM to about 11:10 AM, between about 3:20 PM to about 5:40 PM, and between about 9:50 PM to about 10:53 PM, respectively.

TABLE 39A
Treatment Substance Ingested Not
at 60 Minutes Before Measurements

| | Time of B Score | Liquid Used to Help | |
|---|---|---|---|
| | Measurement | Ingest Treatment | Timeframe of |
| Example | (Minutes) | Substance | Example |
| T39: 1 | 80 | Water | Afternoon |
| T39: 2 | 70 | Wine | Evening |
| T39: 3 | 65 | Water | Afternoon |
| T39: 4 | 80 | Coffee | Morning |
| T39: 5 | 65 | Water | Afternoon |
| T39: 6 | 65 | Water | Afternoon |
| T39: 7 | 65 | Water | Afternoon |
| T39: 8 | 63 | Coffee | Morning |
| T39: 9 | 40 | Coffee | Morning |
| T39: 10 | 62 | Coffee | Morning |
| T39: 11 | 68 | Wine | Evening |
| T39: 12 | 45 | Coffee | Morning |
| T39: 13 | 40 | Coffee | Morning |
| T39: 14A | 30 | Water | Morning |
| T39: 14B | 70 | Water | Morning |
| T39: 15 | 30 | Water | Morning |
| T39: 16 | 30 | Water | Morning |

TABLE 39B
Treatment Substance Ingested Not at 60 Minutes Before Measurements

| | Treatment Substance | | | | | |
|---|---|---|---|---|---|---|
| Example | [Details] | Vendor | mg | C | A | B |
| T39: 1 | NGABA | AACentral | 200 | 1.5 | 8.5 | 9.5 |
| | MagT | Now/PP/SN | 1333 | | | |
| | NAC. SR | Jarrow | 600 | | | |
| | Gly | Now | 1000 | | | |
| | Propolis Extract | Y.S. Farms | 1000 | | | |
| T39: 2 | Val | NutraBio | 3150 | 1.0 | 6.5 | 9.8 |
| | MagT | Now/PP/SN | 1333 | | | |
| | NAC. SR | Jarrow | 600 | | | |
| | Gly | Now | 2000 | | | |
| | 5HTP | Bluebonnet | 100 | | | |
| T39: 3 | Leu | MHP | 2200 | 1.5 | 5.5 | 5.5 |
| | Ile | MHP | 550 | | | |
| | Val | MHP | 550 | | | |
| | Pyridoxine | MHP | 1.5 | | | |
| | Riboflavin | MHP | 1.7 | | | |
| | NAC. SR | Jarrow | 600 | | | |
| | MagT | DBest | 4168 | | | |
| | Gly | Now | 1000 | | | |
| T39: 4 | Val | NutraBio | 4500 | 1.2 | 8.5 | 9.5 |
| | NAC. SR | Jarrow | 600 | | | |
| | MagT | DBest | 4168 | | | |
| | Gly | Now | 1000 | | | |
| T39: 5 | NGABA | AACentral | 200 | 1.8 | 8.5 | 9.8 |
| | MagT | DBest | 2084 | | | |
| | NAC. P | WFoods | 500 | | | |
| | Gly | Now | 1000 | | | |
| T39: 6 | NGABA | AACentral | 200 | 1.8 | 7.5 | 7.5 |
| | MagT | DBest | 2084 | | | |
| | NAC. SR | Jarrow | 600 | | | |
| | Gly | Now | 1000 | | | |
| T39: 7 | NGABA | AACentral | 200 | 1.8 | 7.5 | 8.5 |
| | MagT | DBest | 2084 | | | |
| | NAC. SR | Jarrow | 600 | | | |
| | Gly | Now | 1000 | | | |
| T39: 8 | Val | Now | 813 | 1.2 | 7.5 | 8.5 |
| | Ile | Now | 813 | | | |
| | Leu | Now | 2000 | | | |
| T39: 9 | Val | NutraBio | 3600 | 1.0 | 8.5 | 8.5 |
| | MagT | DBest | 3126 | | | |
| | NAC. P | WFoods | 500 | | | |
| | Oleamide | Liftmode | 100 | | | |
| T39: 10 | Val | BulkSupps | 3600 | 1.8 | 8.8 | 8.8 |
| | MagT | DBest | 3126 | | | |
| | NAC. P | WFoods | 500 | | | |
| | D-Asp | BiotechN | 750 | | | |
| T39: 11 | NGABA | AACentral | 200 | 1.5 | 8.5 | 10.5 |
| | NAC. P | WFoods | 500 | | | |
| | MagT | DBest | 2084 | | | |
| T39: 12 | Val | BulkSupps | 5040 | 1.8 | 7.5 | 8.5 |
| | MagT | DBest | 3126 | | | |
| | NGABA | AACentral | 100 | | | |
| | NAC. P | WFoods | 500 | | | |
| | Rosmarinic Acid | LifeLink | 70 | | | |
| | Grape SeedL | LifeLink | 42 | | | |
| T39: 13 | Phenibut [Fine | Liftmode | 450 | 1.5 | 6.5 | 6.5 |
| | Crystals, No Capsule] | | | | | |
| T39: 14A | Val | Bulksupps | 5040 | 1.2 | 9.2 | 9.2 |
| | NAC. P | WFoods | 500 | | | |
| | 95% PiperineC | Carlyle | 30 | | | |
| T39: 14B | N/A (70 Minutes After | See T39: 14A | | 1.0 | 8.2 | 8.2 |
| | Initial Ingestion of | | | | | |
| | Treatment Substances | | | | | |
| | in Example T39:14A | | | | | |
| | Sexual Functions | | | | | |
| | Measured Again) | | | | | |
| T39: 15 | Val | Bulksupps | 5040 | 2.0 | 9.2 | 9.2 |
| | NAC. P | WFoods | 500 | | | |
| | 95% PiperineC | Carlyle | 30 | | | |
| | Saffron Extract | PipingRock | 177 | | | |
| | MagT | Now/PP/SN | 2666 | | | |
| | Lemon Balm | OWH | 1125 | | | |
| | NGABA | AACentral | 500 | 2.0 | 9.5 | 9.2 |
| T39: 16 | NAC. P | WFoods | 500 | | | |

TABLE 39C
Treatment Substance Ingested Not
at 60 Minutes Before Measurements

| Example | C | CT | A | B | BM | BS | D | DE |
|---|---|---|---|---|---|---|---|---|
| T39: 1 | 1.5 | No | 8.5 | 9.5 | N/A | N/A | 3.0 | 2.0 |
| T39: 2 | 1.0 | No | 6.5 | 9.8 | N/A | N/A | 3.0 | 2.0 |
| T39: 3 | 1.5 | No | 5.5 | 5.5 | N/A | N/A | 3.0 | 2.0 |
| T39: 4 | 1.2 | No | 8.5 | 9.5 | N/A | N/A | 3.0 | 2.0 |
| T39: 5 | 1.8 | No | 8.5 | 9.8 | N/A | N/A | 3.0 | 2.0 |
| T39: 6 | 1.8 | No | 7.5 | 7.5 | N/A | N/A | 3.0 | 2.0 |
| T39: 7 | 1.8 | No | 7.5 | 8.5 | N/A | N/A | 2.0 | 2.0 |
| T39: 8 | 1.2 | No | 7.5 | 8.5 | N/A | N/A | 3.0 | 2.0 |
| T39: 9 | 1.0 | No | 8.5 | 8.5 | N/A | N/A | 3.0 | 2.0 |
| T39: 10 | 1.8 | No | 8.8 | 8.8 | 8.8 | N/A | 4.0 | 2.0 |
| T39: 11 | 1.5 | No | 8.5 | 10.5 | N/A | N/A | 3.0 | 2.0 |
| T39: 12 | 1.8 | No | 7.5 | 8.5 | N/A | N/A | 4.0 | 2.0 |
| T39: 13 | 1.5 | No | 6.5 | 6.5 | N/A | N/A | 4.0 | 2.0 |
| T39: 14A | 1.2 | No | 9.2 | 9.2 | 9.2 | N/A | 3.0 | 3.0 |
| T39: 14B | 1.0 | No | 8.2 | 8.2 | 4.5 | N/A | 3.0 | 1.0 |
| T39: 15 | 2.0 | No | 9.2 | 9.2 | 4.5 | N/A | 3.0 | 2.0 |
| T39: 16 | 2.0 | No | 9.5 | 9.2 | 6.5 | N/A | 3.0 | 2.0 |

Table 40A shows working examples of measurements of sexual function scores after ingestion of two or more doses of one or more treatment substances and the various beverages used to ingest the treatment substances. Table 40B and Table 40C show the same working examples with measurements of the TSF for the A, B, and C scores with additional sexual function measurements shown at Table 40C. In some examples a second measurement of sexual functions was recorded, with the example of first measurement listed as “A” and the example of the second measurement listed as “B.” Examples measured in the morning and afternoon had the same dietary vitamins, nutritional supplements and food ingested as described for Table 37A and Table 36A, respectively, unless otherwise noted. Examples measured in afternoon were at about 5:20 PM, and examples measured in morning were between about 8:05 AM to about 10:10 AM, with example T40:4B measured at about 12:05 PM.

TABLE 40A
Multiple Doses of One or More Treatment Substances

| | Time of B Score | | |
|---|---|---|---|
| | Measurement from | | |
| | the First Dose | Liquid Used to Help | Time- |
| | of Treatment | Ingest Treatment | frame of |
| Example | Substance (Minutes) | Substance | Example |
| T40: 1 | 70 | Coffee | Morning |
| T40: 2 | 90 | Water | Afternoon |
| T40: 3A | 79 | Coffee | Morning |
| T40: 3B | 139 | Coffee | Morning |
| T40: 4A | 60 | Coffee | Morning |
| T40: 4B | 192 | Coffee | Morning |
| T40: 5 | 60 | Coffee | Morning |
| T40: 6 | 80 | Coffee | Morning |
| T40: 7 | 85 | Coffee | Morning |
| T40: 8 | 71 | Coffee | Morning |
| T40: 9 | 70 | Coffee | Morning |
| T40: 10 | 60 | Coffee | Morning |
| T40: 11 | 120 | Coffee | Morning |
| T40: 12 | 90 | Water | Afternoon |
| T40: 13 | 60 | Coffee | Morning |

TABLE 40B
Multiple Doses of One or More Treatment Substances

| | | Treatment | | | | | |
|---|---|---|---|---|---|---|---|
| | Time Ingested Before | Substance | | | | | |
| Example | B Score Measurement | [Details] | Vendor | mg | C | A | B |
| T40: 1 | 70 Minutes | Val | NutraBio | 3600 | 2.0 | 9.5 | 10.5 |
| | | MagT | DBest | 2084 | | | |
| | | NAC. SR | Jarrow | 600 | | | |
| | 55 Minutes | Val | NutraBio | 3600 | | | |
| | | MagT | DBest | 2084 | | | |
| | | NAC. SR | Jarrow | 600 | | | |
| T40: 2 | 90 Minutes | Val | NutraBio | 1800 | 2.0 | 8.5 | 9.5 |
| | | MagT | Now/PP/SN | 667 | | | |
| | | NAC. SR | Jarrow | 600 | | | |
| | | Gly | Now | 1000 | | | |
| | 60 Minutes | Val | NutraBio | 1800 | | | |
| | | MagT | Now/PP/SN | 667 | | | |
| | | NAC. SR | Jarrow | 600 | | | |
| | | Gly | Now | 1000 | | | |
| T40: 3A | 79 Minutes | NGABA | AACentral | 200 | 3.0 | 9.5 | 9.5 |
| | | MagT | Now/PP/SN | 1333 | | | |
| | | NAC. SR | Jarrow | 600 | | | |
| | | Gly | Now | 1000 | | | |
| | 59 Minutes | Val | NutraBio | 3150 | | | |
| | | MagT | Now/PP/SN | 1333 | | | |
| | | NAC. SR | Jarrow | 600 | | | |
| | | Gly | Now | 1000 | | | |
| T40: 3B | 139 Minutes After | N/A | N/A | | 2.0 | 8.8 | 9.8 |
| | Ingestion of the First Dose | | | | | | |
| | of Treatment Substances in | | | | | | |
| | Example T40: 3A Sexual | | | | | | |
| | Functions Measured | | | | | | |
| T40: 4A | 60 Minutes | NGABA | AACentral | 200 | 1.5 | 9.5 | 9.8 |
| | | MagT | Now/PP/SN | 1333 | | | |
| | | NAC. SR | Jarrow | 600 | | | |
| | | Gly | Now | 1000 | | | |
| | 40 Minutes | Val | NutraBio | 3150 | | | |
| | | MagT | Now/PP/SN | 1333 | | | |
| | | NAC. SR | Jarrow | 600 | | | |
| | | Gly | Now | 1000 | | | |
| T40: 4B | 37 Minutes After Example | Ile | ON Brand | 750 | 1.5 | 9.5 | 9.8 |
| | T40: 4A Measurements | Leu | ON Brand | 1500 | | | |
| | Food (Carbohydrate/ | Val | ON Brand | 750 | | | |
| | Protein/Fat) Ingested with | Gln | WFoods | 1000 | | | |
| | Treatment Substances (Ile, | | | | | | |
| | Leu, Val, Gln); 192 | | | | | | |
| | Minutes After Initial | | | | | | |
| | Ingestion of Treatment | | | | | | |
| | Substances in Example | | | | | | |
| | T40: 4A Sexual Functions | | | | | | |
| | Measured | | | | | | |
| T40: 5 | 60 Minutes | Val | NutraBio | 3150 | 1.5 | 9.5 | 9.8 |
| | | MagT | Now/PP/SN | 1333 | | | |
| | | NAC. SR | Jarrow | 600 | | | |
| | | Gly | Now | 1000 | | | |
| | 40 Minutes | NGABA | AACentral | 200 | | | |
| | | MagT | Now/PP/SN | 1333 | | | |
| | | NAC. SR | Jarrow | 600 | | | |
| | | Gly | Now | 1000 | | | |
| T40: 6 | 80 Minutes | Val | NutraBio | 3150 | 2.5 | 8.5 | FE |
| | | MagT | DBest | 2084 | | | |
| | | NAC. SR | Jarrow | 1200 | | | |
| | 50 Minutes | NGABA | AACentral | 200 | | | |
| | | MagT | DBest | 1042 | | | |
| | | NAC. P | WFoods | 500 | | | |
| T40: 7 | 85 Minutes | NGABA | AACentral | 200 | 1.5 | 9.5 | 10.5 |
| | | MagT | DBest | 1042 | | | |
| | | NAC. SR | Jarrow | 600 | | | |
| | 60 Minutes | Val | NutraBio | 3150 | | | |
| | | MagT | DBest | 2084 | | | |
| | | NAC. P | WFoods | 1000 | | | |
| T40: 8 | 71 Minutes | Val | NutraBio | 3150 | 2.5 | 10.5 | 8.5 |
| | | NAC. P | WFoods | 1000 | | | |
| | 51 Minutes | Val | NutraBio | 3150 | | | |
| | | NAC. P | WFoods | 1000 | | | |
| T40: 9 | 70 Minutes | Val | NutraBio | 3600 | 2.0 | 9.5 | 9.5 |
| | | MagT | DBest | 2084 | | | |
| | 55 Minutes | Val | NutraBio | 3600 | | | |
| | | MagT | DBest | 2084 | | | |
| T40: 10 | 60 Minutes | Val | NutraBio | 3600 | 1.8 | 9.5 | 10.5 |
| | | MagT | DBest | 3126 | | | |
| | 35 Minutes | NAC. P | WFoods | 500 | | | |
| | | Gly | Now | 1000 | | | |
| T40: 11 | 120 Minutes | Caffeine* | Nutrakey | 100 | 1.2 | 10.5 | 10.5 |
| | 60 Minutes | Val | NutraBio | 3600 | | | |
| | | MagT | DBest | 3126 | | | |
| | | NAC. P | WFoods | 500 | | | |
| | | D-Asp | BiotechN | 750 | | | |
| T40: 12 | 90 Minutes | NGABA | AACentral | 100 | 3.5 | 9.5 | 9.8 |
| | | NAC. SR | Jarrow | 600 | | | |
| | | MagT | Now/PP/SN | 667 | | | |
| | | Gly | Now | 1000 | | | |
| | 60 Minutes | NGABA | AACentral | 100 | | | |
| | | NAC. SR | Jarrow | 600 | | | |
| | | MagT | Now/PP/SN | 667 | | | |
| | | Gly | Now | 1000 | | | |
| T40: 13 | 60 Minutes | Val | BulkSupps | 5760 | 1.2 | 10.2 | 10.2 |
| | | NAC. P | BulkSupps | 720 | | | |
| | 30 Minutes | Pramiracetam | NoopDepot | 625 | | | |

*About half of the powder removed from capsule

TABLE 40C
Multiple Doses of One or More Treatment Substances

| Example | C | CT | A | B | BM | BS | D | DE |
|---|---|---|---|---|---|---|---|---|
| T40: 1 | 2.0 | No | 9.5 | 10.5 | N/A | N/A | 3.0 | 2.0 |
| T40: 2 | 2.0 | No | 8.5 | 9.5 | N/A | N/A | 3.0 | 2.0 |
| T40: 3A | 3.0 | No | 9.5 | 9.5 | N/A | N/A | 3.0 | 2.0 |
| T40: 3B | 2.0 | No | 8.8 | 9.8 | N/A | N/A | 3.0 | 2.0 |
| T40: 4A | 1.5 | No | 9.5 | 9.8 | N/A | N/A | 3.0 | 2.0 |
| T40: 4B | 1.5 | No | 9.5 | 9.8 | N/A | N/A | 3.0 | 2.0 |
| T40: 5 | 1.5 | No | 9.5 | 9.8 | N/A | N/A | 3.0 | 2.0 |
| T40: 6 | 2.5 | No | 8.5 | FE | N/A | N/A | FE | FE |
| T40: 7 | 1.5 | No | 9.5 | 10.5 | N/A | N/A | 4.0 | 2.0 |
| T40: 8 | 2.5 | No | 10.5 | 8.5 | N/A | N/A | 3.0 | 2.0 |
| T40: 9 | 2.0 | No | 9.5 | 9.5 | N/A | N/A | 3.0 | 2.0 |
| T40: 10 | 1.8 | No | 9.5 | 10.5 | 0.0 | N/A | 4.0 | 2.0 |
| T40: 11 | 1.2 | No | 10.5 | 10.5 | 0.0 | N/A | 4.0 | 2.0 |
| T40: 12 | 3.5 | No | 9.5 | 9.8 | N/A | N/A | 3.0 | 2.0 |
| T40: 13 | 1.2 | No | 10.2 | 10.2 | 0.0 | FT | 4.0 | 2.0 |

Table 41A shows working examples of measurements of the TSF for the A, B, and C scores after ingestion of one or more treatment substances, wherein the treatment substances were ingested 60 minutes before the measurements. Table 41B shows the same working examples with additional sexual function measurements listed. Unless otherwise noted herein, water rather than coffee was ingested to remove the variable of caffeine as a treatment substance, and these working examples were conducted after a full night's sleep and no noticeable effects of alcohol consumption the previous evening, if alcohol was ingested, to the remove and/or reduce the variables of sleep deprivation and alcohol. Noted herein: example T41:142 was conducted after sleep deprivation the night before, but there was no noticeable effects from alcohol consumption the previous evening; examples T41:67 and T41:68 had the treatment substance dissolved under the tongue without ingesting water per vendor's instructions; and examples T41:71 and T41:72 had drops in water held in the mouth for 30 seconds per vendor's instructions before swallowing. No food, dietary vitamins or nutritional supplements were ingested the day of the measurements until after the measurements were taken. Examples were made in the morning between about 8:51 AM to about 11:51 AM, though the times in the morning for Examples T41:140 and T41:141 were not recorded. A treatment substance, other than Val and NAC, was ingested at a low dose, generally the dose recommended by the vendor, to measure any side effect (e.g., an adverse event). The same treatment substance was ingested at a higher dose, generally 3 times the low dose amount, to measure any side effect. Val and NAC, a combination of treatment substances that typically produces positive effects on TSF after ingestion (e.g., A and B scores often 9 or above when ingested together without another treatment substance) were selected for combinations with one or more of the other treatment substances being evaluated. The other treatment substance was typically ingested at the higher dose amount, to evaluate the treatment substance's effect on increasing/decreasing the positive effects of Val and NAC on SRSS and sexual functions. It was contemplated that a different positive GABAergic (e.g., NGABA, Leu, Ile) could be used as a substitute for Val in this method of evaluation of an additional treatment substance. GABA is less preferred as a substitute positive GABAergic, as GABA has a reduced ability to cross the BBB relative to a preferred treatment substance such as NGABA/BCAAs, and in early treatment substance evaluations GABA (e.g., 750 mg dose; vendor PuritanP) produced mild improvement in TSF relative to Val and the preferred substitute positive GABAergic treatment substance(s).

TABLE 41A
Treatment Substance Ingested with Water in the Morning

| | Treatment Substance | | | | | |
|---|---|---|---|---|---|---|
| Example | [Details] | Vendor | mg | C | A | B |
| T41: 1 | Prickly Ash | SOTT | 800 | 1.0 | 3.5 | 1.5 |
| T41: 2 | Incarvillea sinensis | ABotanicals | 200 | 0.0 | 3.5 | 4.5 |
| | [Tablet CW**] | | | | | |
| T41: 3 | Gotu Kola | SwansonHP | 60 | 0.0 | 0.0 | 0.0 |
| T41: 4 | Fisetin | SwansonHP | 100 | 0.0 | 3.5 | 2.5 |
| T41: 5 | DHEA | SwansonHP | 25 | 0.0 | 3.5 | 3.5 |
| T41: 6 | Lobelia | SOTT | 800 | 0.0 | 5.5 | 5.5 |
| T41: 7 | Lemon Balm Extract | NoopDepot | 600 | 1.0 | 6.5 | 6.5 |
| T41: 8 | 60% Caprylic Acid | Now | 1000 | 1.5 | 2.5 | 2.5 |
| T41: 9 | Uridine | Double Wood | 300 | 0.0 | 3.5 | 3.5 |
| T41: 10 | 99% Theobromine | Barlowe | 400 | 1.0 | 7.5 | 3.5 |
| T41: 11 | Oxaloacetate | Terra Bio | 100 | 0.2 | 6.5 | 3.5 |
| | Ascorbic Acid | Terra Bio | 150 | | | |
| T41: 12 | Licorice | Bixa | 450 | 0.0 | 4.5 | 3.5 |
| T41: 13 | 50% SAMe | SwansonHP | 400 | 0.5 | 6.5 | 0.0 |
| | [2 Tablets CW**] | | | | | |
| T41: 14 | Mucin/Sialac | EcoFormulas | 500 | 1.0 | 5.5 | 5.5 |
| | Acid Preparation | | | | | |
| T41: 15 | Chamomile | Barlowe | 1200 | 0.0 | 4.5 | 0.0 |
| T41: 16 | Manganese Bisglycinate | Thorne | 15 | 0.2 | 1.5 | 0.0 |
| T41: 17 | 50% Hydroxycitric Acid | Solgar | 500 | 0.2 | 0.0 | 0.0 |
| T41: 18 | Ascorbyl Palmitate, | SNaturals | 500 | 0.2 | 4.5 | 4.5 |
| | Calcium Phosphate | | | | | |
| | [Tablet CW**] | | | | | |
| T41: 19 | Asp | SwansonHP | 500 | 0.2 | 3.5 | 2.5 |
| T41: 20 | Chlorpheniramine | Walmart | 4 | 0.2 | 1.5 | 0.0 |
| T41: 21 | Diphenhydramine | Walmart | 25 | 0.0 | 2.5 | 0.0 |
| T41: 22 | Dihydromyricetin | Pure Noops | 300 | 0.5 | 1.5 | 1.5 |
| T41: 23 | Prickly Ash | SOTT | 2400 | 1.2 | 2.5 | 0.0 |
| T41: 24 | Incarvillea sinensis | ABotanicals | 600 | 0.5 | 4.5 | 2.5 |
| | [3 Tablets CW**] | | | | | |
| T41: 25 | Gotu Kola | SwansonHP | 180 | 0.2 | 0.0 | 0.0 |
| T41: 26 | Fisetin | SwansonHP | 300 | 0.2 | 4.5 | 3.5 |
| T41: 27 | DHEA | SwansonHP | 75 | 0.5 | 2.5 | 2.5 |
| T41: 28 | Lobelia | SOTT | 2400 | 0.5 | 4.5 | 3.5 |
| T41: 29 | Lemon Balm Extract | NoopDepot | 1800 | 0.8 | 4.5 | 4.5 |
| T41: 30 | 60% Caprylic Acid | Now | 3000 | 1.2 | 2.5 | 2.5 |
| T41: 31 | Uridine | Double Wood | 900 | 1.0 | 0.0 | 0.0 |
| T41: 32 | 99% Theobromine | Barlowe | 1200 | 1.0 | 7.5 | 6.5 |
| T41: 33 | Oxaloacetate | Terra Bio | 300 | 1.0 | 2.5 | 2.5 |
| | Ascorbic Acid | Terra Bio | 450 | | | |
| T41: 34 | Licorice | Bixa | 1350 | 0.5 | 3.5 | 3.5 |
| T41: 35 | 50% SAMe | SwansonHP | 1200 | 1.0 | 4.5 | 4.5 |
| T41: 36 | Mucin/Sialac | EcoFormulas | 1500 | 0.5 | 4.5 | 4.5 |
| | Acid Preparation | | | | | |
| T41: 37 | Chamomile | Barlowe | 3600 | 1.0 | 4.5 | 4.5 |
| T41: 38 | Manganese Bisglycinate | Thorne | 45 | 0.5 | 3.5 | 3.5 |
| T41: 39 | 50% Hydroxycitric Acid | Solgar | 1500 | 0.5 | 2.5 | 2.5 |
| T41: 40 | Ascorbyl Palmitate, | SNaturals | 1500 | 0.2 | 2.5 | 2.5 |
| | Calcium Phosphate | | | | | |
| T41: 41 | Asp | SwansonHP | 1500 | 0.2 | 2.5 | 1.5 |
| T41: 42 | Dihydromyricetin | Pure Noops | 900 | 0.5 | 5.5 | 5.5 |
| T41: 43 | Bahera | Amalth | 500 | 1.2 | 6.5 | 6.5 |
| T41: 44 | Bahera | Amalth | 1500 | 1.0 | 4.5 | 4.5 |
| T41: 45 | Hops [Tablet CW**] | ABotanicals | 100 | 0.0 | 4.5 | 4.5 |
| T41: 46 | Hops [3 Tablets CW**] | ABotanicals | 300 | 0.2 | 4.5 | 4.5 |
| T41: 47 | Palmitoylethanolamide | Supersmart | 1200 | 0.5 | 2.5 | 2.5 |
| T41: 48 | Palmitoylethanolamide | Supersmart | 3600 | 0.2 | 0.0 | 0.0 |
| T41: 49 | His | SwansonHP | 500 | 0.5 | 4.5 | 4.5 |
| T41: 50 | His | SwansonHP | 1500 | 1.0 | 5.5 | 5.5 |
| T41: 51 | Cloves Extract | PipingRock | 100 | 0.8 | 6.5 | 6.5 |
| T41: 52 | Cloves Extract | PipingRock | 300 | 1.2 | 6.5 | 6.5 |
| T41: 53 | Aminoguanidine | Supersmart | 75 | 0.5 | 5.5 | 5.5 |
| T41: 54 | Aminoguanidine | Supersmart | 225 | 1.0 | 6.5 | 6.5 |
| T41: 55 | Ginkgo | MRM | 60 | 0.2 | 4.5 | 4.5 |
| T41: 56 | Ginkgo | MRM | 180 | 0.5 | 5.5 | 5.5 |
| T41: 57 | Pueraria | SMS | 250 | 0.5 | 6.5 | 6.5 |
| | Mirifica Extract | | | | | |
| T41: 58 | Pueraria | SMS | 750 | 0.8 | 5.5 | 5.5 |
| | Mirifica Extract | | | | | |
| T41: 59 | Valerian Root Extract | SwansonHP | 200 | 0.2 | 3.5 | 3.5 |
| | Valerian Root | SwansonHP | 350 | | | |
| T41: 60 | Valerian Root Extract | SwansonHP | 800 | 1.5 | 7.5 | 7.5 |
| | Valerian Root | SwansonHP | 1400 | | | |
| T41: 61 | 4-DMA-7,8-DHF | NoopDepot | 8 | 0.2 | 2.5 | 2.5 |
| T41: 62 | 4-DMA-7,8-DHF | NoopDepot | 24 | 1.0 | 6.5 | 6.5 |
| T41: 63 | Palmitoylethanolamide | NoopDepot | 400 | 0.5 | 1.5 | 1.5 |
| T41: 64 | Palmitoylethanolamide | NoopDepot | 1200 | 0.5 | 3.5 | 3.5 |
| T41: 65 | 6% Beta-Caryophyllene | NoopDepot | 500 | 0.0 | 2.5 | 2.5 |
| T41: 66 | 6% Beta-Caryophyllene | NoopDepot | 1500 | 0.0 | 2.5 | 2.5 |
| T41: 67 | Belladonna | Boiron | 0.44 | 0.2 | 3.5 | 3.5 |
| T41: 68 | Belladonna | Boiron | 2.2 | 0.8 | 3.5 | 3.5 |
| T41: 69 | Gastrodia | HawaiiP | 970 | 0.0 | 3.5 | 3.5 |
| T41: 70 | Gastrodia | HawaiiP | 2910 | 1.0 | 6.5 | 6.5 |
| T41: 71 | BDNF* | GUNA | 20 Drops | 0.2 | 4.5 | 4.5 |
| T41: 72 | BDNF* | GUNA | 60 Drops | 0.5 | 3.5 | 3.5 |
| T41: 73 | 98% Phloretin | KAN | 20 | 1.2 | 5.5 | 5.5 |
| T41: 74 | 98% Phloretin | KAN | 40 | 0.8 | 5.5 | 5.5 |
| T41: 75 | Kava Liquid | Herb Pharm | 580 | 0.5 | 5.5 | 5.5 |
| T41: 76 | Kava Liquid | Herb Pharm | 1160 | 0.8 | 6.5 | 6.5 |
| T41: 77 | Red Clover | KAN | 400 | 1.0 | 5.5 | 5.5 |
| T41: 78 | Red Clover | KAN | 1200 | 1.0 | 6.5 | 5.5 |
| T41: 79 | NMDA | BotanyBio | 100 | 1.0 | 5.5 | 5.5 |
| T41: 80 | NMDA | BotanyBio | 300 | 0.5 | 5.5 | 5.5 |
| T41: 81 | Apigenin | SwansonHP | 50 | 0.5 | 6.5 | 6.5 |
| T41: 82 | Apigenin | SwansonHP | 150 | 1.0 | 5.5 | 5.5 |
| T41: 83 | 1% Spermidine | Supersmart | 150 | 1.0 | FO | FO |
| T41: 84 | 1% Spermidine | Supersmart | 450 | 0.5 | 2.5 | 2.5 |
| T41: 85 | Chrysin | SwansonHP | 500 | 0.2 | 5.5 | 4.5 |
| | 95% PiperineS | SwansonHP | 5 | | | |
| T41: 86 | Chrysin | SwansonHP | 1500 | 0.8 | 6.5 | 5.5 |
| | 95% PiperineS | SwansonHP | 15 | | | |
| T41: 87 | 99% Rauwolscine | GorillaM | 3 | 1.8 | 6.5 | 6.5 |
| T41: 88 | Hesperidin | SwansonHP | 500 | 1.2 | 2.5 | 2.5 |
| T41: 89 | Hesperidin | SwansonHP | 1500 | 1.5 | 5.5 | 5.5 |
| T41: 90 | 80% Luteolin | Supersmart | 63 | 0.5 | 4.5 | 3.5 |
| T41: 91 | 80% Luteolin | Supersmart | 188 | 1.5 | 5.5 | 4.5 |
| T41: 92 | Phe | Now | 500 | 0.2 | 2.5 | 2.5 |
| T41: 93 | Phe | Now | 1500 | 1.5 | 5.5 | 4.5 |
| T41: 94 | D-Phe | DBest | 500 | 0.5 | 5.5 | 5.5 |
| T41: 95 | D-Phe | DBest | 1500 | 0.8 | 2.5 | 2.5 |
| T41: 96 | Turkey Rhubarb | SwansonHP | 500 | 1.0 | 5.5 | 4.5 |
| T41: 97 | Turkey Rhubarb | SwansonHP | 1500 | 1.8 | 6.5 | 5.5 |
| T41: 98 | Mangosteen | SwansonHP | 500 | 0.8 | 4.5 | 3.5 |
| T41: 99 | Mangosteen | SwansonHP | 1500 | 1.0 | 6.5 | 6.5 |
| T41: 100 | Propionyl-Carnitine | CVRL | 333 | 0.0 | 3.5 | 2.5 |
| T41: 101 | Propionyl-Carnitine | CVRL | 999 | 0.2 | 6.5 | 5.5 |
| T41: 102 | Alpha-Glycosyl | Integrative | 33 | 1.2 | 6.5 | 6.5 |
| | Isoquercitrin | | | | | |
| T41: 103 | Alpha-Glycosyl | Integrative | 99 | 1.5 | 6.5 | 6.5 |
| | Isoquercitrin | | | | | |
| T41: 104 | Milk Thistle Extract | PipingRock | 250 | 0.2 | 5.5 | 4.5 |
| T41: 105 | Milk Thistle Extract | PipingRock | 750 | 0.5 | 7.5 | 5.5 |
| T41: 106 | Kudzu | NaturesWay | 1226 | 0.2 | 2.5 | 0.0 |
| T41: 107 | Kudzu | NaturesWay | 3678 | 0.8 | 5.5 | 4.5 |
| T41: 108 | 55% Caprylic Acid and | NatureNut | 3000 | 0.8 | 3.5 | 3.5 |
| | 35% Capric Acid | | | | | |
| T41: 109 | Chamomile Liquid* | PipingRock | 1 mL | 0.5 | 5.5 | 4.5 |
| T41: 110 | Chamomile Liquid* | PipingRock | 3 mL | 1.2 | 7.5 | 7.5 |
| T41: 111 | Val | Bulksupps | 5040 | 1.5 | 9.2 | 0.0 |
| | NAC. P | WFoods | 500 | | | |
| | Valerian Root Extract | SwansonHP | 800 | | | |
| | Valerian Root | SwansonHP | 1400 | | | |
| T41: 112 | Val | Bulksupps | 5040 | 1.2 | 9.2 | 8.8 |
| | NAC. P | WFoods | 500 | | | |
| | Lemon Balm Extract | NoopDepot | 1800 | | | |
| T41: 113 | Val | Bulksupps | 5040 | 1.5 | 9.2 | 9.2 |
| | NAC. P | WFoods | 500 | | | |
| | 99% Theobromine | Barlowe | 1200 | | | |
| T41: 114 | Val | Bulksupps | 5040 | 1.0 | 4.5 | 3.5 |
| | NAC. P | WFoods | 500 | | | |
| | Lobelia | SOTT | 2400 | | | |
| T41: 115 | Val | Bulksupps | 5040 | 1.2 | 8.5 | 7.5 |
| | NAC. P | WFoods | 500 | | | |
| | Oxaloacetate | Terra Bio | 300 | | | |
| | Ascorbic Acid | Terra Bio | 450 | | | |
| T41: 116 | Val | Bulksupps | 5040 | 1.0 | 6.5 | 5.5 |
| | NAC. P | WFoods | 500 | | | |
| | 50% SAMe | SwansonHP | 1200 | | | |
| T41: 117 | Val | Bulksupps | 5040 | 0.8 | 7.2 | 0.0 |
| | NAC. P | WFoods | 500 | | | |
| | Mucin/Sialac Acid Preparation | EcoFormulas | 1500 | | | |
| T41: 118 | Val | Bulksupps | 5040 | 1.5 | 6.5 | 6.5 |
| | NAC. P | WFoods | 500 | | | |
| | His | SwansonHP | 1500 | | | |
| T41: 119 | Val | Bulksupps | 5040 | 0.0 | 2.5 | 0.0 |
| | NAC. P | WFoods | 500 | | | |
| | Bahera | Amalth | 1500 | | | |
| T41: 120 | Val | Bulksupps | 5040 | 1.2 | 9.2 | 8.8 |
| | NAC. P | WFoods | 500 | | | |
| | Cloves Extract | PipingRock | 300 | | | |
| T41: 121 | Val | Bulksupps | 5040 | 1.0 | 3.5 | 3.5 |
| | NAC. P | WFoods | 500 | | | |
| | Aminoguanidine | Supersmart | 225 | | | |
| T41: 122 | Val | Bulksupps | 5040 | 0.8 | 8.2 | 7.5 |
| | NAC. P | WFoods | 500 | | | |
| | Ginkgo | MRM | 180 | | | |
| T41: 123 | Val | Bulksupps | 5040 | 1.2 | 8.8 | 7.5 |
| | NAC. P | WFoods | 500 | | | |
| | Pueraria Mirifica | SMS | 750 | | | |
| | Extract | | | | | |
| T41: 124 | Val | Bulksupps | 5040 | 1.2 | 8.5 | 7.5 |
| | NAC. P | WFoods | 500 | | | |
| | 98% Phloretin | KAN | 40 | | | |
| T41: 125 | Val | Bulksupps | 5040 | 1.0 | 8.5 | 8.2 |
| | NAC. P | WFoods | 500 | | | |
| | Kava Liquid | Herb Pharm | 1160 | | | |
| T41: 126 | Val | Bulksupps | 5040 | 0.0 | 3.5 | 3.5 |
| | NAC. P | WFoods | 500 | | | |
| | NMDA | BotanyBio | 300 | | | |
| T41: 127 | Val | Bulksupps | 5040 | 0.0 | 4.5 | 4.5 |
| | NAC. P | WFoods | 500 | | | |
| | Apigenin | SwansonHP | 150 | | | |
| T41: 128 | Val | Bulksupps | 5040 | 1.0 | 9.2 | 8.5 |
| | NAC. P | WFoods | 500 | | | |
| | Chrysin | SwansonHP | 1500 | | | |
| | 95% PiperineS | SwansonHP | 15 | | | |
| T41: 129 | Val | Bulksupps | 5040 | 0.0 | 5.5 | 0.0 |
| | NAC. P | WFoods | 500 | | | |
| | Hesperidin | SwansonHP | 1500 | | | |
| T41: 130 | Val | Bulksupps | 5040 | 0.0 | 4.5 | 4.5 |
| | NAC. P | WFoods | 500 | | | |
| | 80% Luteolin | Supersmart | 188 | | | |
| T41: 131 | Val | Bulksupps | 5040 | 0.0 | 7.5 | 5.5 |
| | NAC. P | WFoods | 500 | | | |
| | Mangosteen | SwansonHP | 1500 | | | |
| T41: 132 | Val | Bulksupps | 5040 | 0.2 | 4.5 | 4.5 |
| | NAC. P | WFoods | 500 | | | |
| | D-Phe | DBest | 500 | | | |
| T41: 133 | Val | Bulksupps | 5040 | 1.0 | 7.5 | 7.5 |
| | NAC. P | WFoods | 500 | | | |
| | D-Phe | DBest | 1500 | | | |
| T41: 134 | Val | Bulksupps | 5040 | 1.2 | 8.8 | 8.5 |
| | NAC. P | WFoods | 500 | | | |
| | Propionyl-Carnitine | CVRL | 999 | | | |
| T41: 135 | Val | Bulksupps | 5040 | 0.2 | 6.5 | 5.5 |
| | NAC. P | WFoods | 500 | | | |
| | Turkey Rhubarb | SwansonHP | 1500 | | | |
| T41: 136 | Val | Bulksupps | 5040 | 0.0 | 3.5 | 3.5 |
| | NAC. P | WFoods | 500 | | | |
| | Alpha-Glycosyl | Integrative | 99 | | | |
| | Isoquercitrin | | | | | |
| T41: 137 | Val | Bulksupps | 5040 | 1.0 | 7.5 | 7.5 |
| | NAC. P | WFoods | 500 | | | |
| | D-Beta- | KetoneAid | 30,000 | | | |
| | Hydroxybutyrate Ester | | | | | |
| | Potassium | KetoneAid | 99 | | | |
| T41: 138 | Val | Bulksupps | 5040 | 0.2 | 9.2 | 9.2 |
| | NAC. P | WFoods | 500 | | | |
| | Ornithine | BulkSupps | 1791 | | | |
| T41: 139 | Val | Bulksupps | 5040 | 0.5 | 0.0 | 0.0 |
| | NAC. P | WFoods | 500 | | | |
| | 1% Spermidine | Supersmart | 450 | | | |
| T41: 140 | Galantamine HBr | Relentless | 8 | 0.8 | 0.0 | 1.5 |
| T41: 141 | Inositol | Now | 500 | 0.0 | 8.8 | 8.5 |
| T41: 142 | Val | Bulksupps | 5040 | 1.2 | 8.5 | 8.2 |
| | NAC. P | WFoods | 500 | | | |
| | 50% Trans-Resveratrol | WFoods | 250 | | | |
| | Ascorbic Acid | IV for Life | 333 | | | |
| | Liposomal Formulation | IV for Life | 117 | | | |
| T41: 143 | Val | Bulksupps | 5040 | 0.2 | 8.2 | 7.5 |
| | NAC. P | WFoods | 500 | | | |
| | Dihydromyricetin | Pure Noops | 900 | | | |
| T41: 144 | Val | Bulksupps | 5040 | 1.2 | 8.8 | 8.5 |
| | NAC. P | PureBulk | 720 | | | |
| | Cat's Claw Stalk | HawaiiP | 983 | | | |
| T41: 145 | Val | Bulksupps | 5040 | 1.2 | 9.2 | 9.2 |
| | NAC. P | PureBulk | 720 | | | |
| | Noopept | HealthN | 90 | | | |
| T41: 146 | Val | Bulksupps | 5040 | 1.2 | 9.5 | 9.2 |
| | NAC. P | PureBulk | 720 | | | |
| | Vanilla* | Adam's | 1 mL | | | |

*Treatment substance amount in different unit other than mg such as liquid drops, mL, etc. as Noted;
**“Tablet(s) CW” means the tablet(s) were chewed before swallowing to promote rapid absorption

TABLE 41B
Treatment Substance Ingested with Water in the Morning

| Example | C | CT | A | B | BM | BS | D | DE |
|---|---|---|---|---|---|---|---|---|
| T41: 1 | 1.0 | No | 3.5 | 1.5 | N/A | N/A | 3.0 | 2.0 |
| T41: 2 | 0.0 | No | 3.5 | 4.5 | 4.5 | N/A | 3.0 | 2.0 |
| T41: 3 | 0.0 | N/A | 0.0 | 0.0 | N/A | N/A | 3.0 | 2.0 |
| T41: 4 | 0.0 | Yes | 3.5 | 2.5 | 0.0 | N/A | 3.0 | 2.0 |
| T41: 5 | 0.0 | N/A | 3.5 | 3.5 | 0.0 | N/A | 3.0 | 2.0 |
| T41: 6 | 0.0 | Yes | 5.5 | 5.5 | 5.5 | N/A | 3.0 | 2.0 |
| T41: 7 | 1.0 | No | 6.5 | 6.5 | 0.0 | N/A | 3.0 | 2.0 |
| T41: 8 | 1.5 | Yes | 2.5 | 2.5 | 0.0 | N/A | 3.0 | 3.0 |
| T41: 9 | 0.0 | Yes | 3.5 | 3.5 | 3.5 | N/A | 3.0 | 2.0 |
| T41: 10 | 1.0 | No | 7.5 | 3.5 | 3.5 | IS | 3.0 | 3.0 |
| T41: 11 | 0.2 | No | 6.5 | 3.5 | 0.0 | N/A | 3.0 | 2.0 |
| T41: 12 | 0.0 | Yes | 4.5 | 3.5 | 0.0 | SL | 3.0 | 2.0 |
| T41: 13 | 0.5 | Yes | 6.5 | 0.0 | 0.0 | IS | 3.0 | 2.0 |
| T41: 14 | 1.0 | No | 5.5 | 5.5 | 3.5 | N/A | 3.0 | 2.0 |
| T41: 15 | 0.0 | Yes | 4.5 | 0.0 | 0.0 | IS | 3.0 | 2.0 |
| T41: 16 | 0.2 | No | 1.5 | 0.0 | 0.0 | IS | 3.0 | 2.0 |
| T41: 17 | 0.2 | Yes | 0.0 | 0.0 | 0.0 | N/A | 3.0 | 2.0 |
| T41: 18 | 0.2 | Yes | 4.5 | 4.5 | 4.5 | N/A | 3.0 | 2.0 |
| T41: 19 | 0.2 | Yes | 3.5 | 2.5 | 2.5 | N/A | 3.0 | 2.0 |
| T41: 20 | 0.2 | Yes | 1.5 | 0.0 | 0.0 | N/A | 3.0 | 2.0 |
| T41: 21 | 0.0 | No | 2.5 | 0.0 | 0.0 | IS | 3.0 | 2.0 |
| T41: 22 | 0.5 | No | 1.5 | 1.5 | 0.0 | N/A | 3.0 | 2.0 |
| T41: 23 | 1.2 | No | 2.5 | 0.0 | 0.0 | IS | 3.0 | 2.0 |
| T41: 24 | 0.5 | No | 4.5 | 2.5 | N/A | N/A | 3.0 | 2.0 |
| T41: 25 | 0.2 | No | 0.0 | 0.0 | 0.0 | N/A | 3.0 | 2.0 |
| T41: 26 | 0.2 | Yes | 4.5 | 3.5 | 0.0 | FT | 3.0 | 2.0 |
| T41: 27 | 0.5 | No | 2.5 | 2.5 | 2.5 | N/A | 3.0 | 2.0 |
| T41: 28 | 0.5 | No | 4.5 | 3.5 | 0.0 | N/A | 3.0 | 2.0 |
| T41: 29 | 0.8 | No | 4.5 | 4.5 | 4.5 | N/A | 3.0 | 2.0 |
| T41: 30 | 1.2 | No | 2.5 | 2.5 | 0.0 | N/A | 3.0 | 2.0 |
| T41: 31 | 1.0 | No | 0.0 | 0.0 | 0.0 | N/A | 3.0 | 2.0 |
| T41: 32 | 1.0 | No | 7.5 | 6.5 | 3.5 | FT | 3.0 | 2.0 |
| T41: 33 | 1.0 | No | 2.5 | 2.5 | 0.0 | N/A | 3.0 | 2.0 |
| T41: 34 | 0.5 | Yes | 3.5 | 3.5 | 0.0 | IS | 3.0 | 2.0 |
| T41: 35 | 1.0 | No | 4.5 | 4.5 | 4.5 | N/A | 3.0 | 2.0 |
| T41: 36 | 0.5 | No | 4.5 | 4.5 | 0.0 | FT | 3.0 | 2.0 |
| T41: 37 | 1.0 | No | 4.5 | 4.5 | 0.0 | FT | 3.0 | 2.0 |
| T41: 38 | 0.5 | Yes | 3.5 | 3.5 | 0.0 | N/A | 3.0 | 2.0 |
| T41: 39 | 0.5 | Yes | 2.5 | 2.5 | 0.0 | N/A | 3.0 | 2.0 |
| T41: 40 | 0.2 | Yes | 2.5 | 2.5 | 0.0 | N/A | 3.0 | 2.0 |
| T41: 41 | 0.2 | Yes | 2.5 | 1.5 | 1.5 | N/A | 3.0 | 2.0 |
| T41: 42 | 0.5 | Yes | 5.5 | 5.5 | 3.5 | N/A | 3.0 | 2.0 |
| T41: 43 | 1.2 | No | 6.5 | 6.5 | 0.0 | N/A | 3.0 | 2.0 |
| T41: 44 | 1.0 | Yes | 4.5 | 4.5 | 2.5 | N/A | 3.0 | 2.0 |
| T41: 45 | 0.0 | Yes | 4.5 | 4.5 | 4.5 | N/A | 3.0 | 2.0 |
| T41: 46 | 0.2 | Yes | 4.5 | 4.5 | 3.5 | N/A | 3.0 | 2.0 |
| T41: 47 | 0.5 | Yes | 2.5 | 2.5 | 2.5 | N/A | 3.0 | 2.0 |
| T41: 48 | 0.2 | Yes | 0.0 | 0.0 | 0.0 | N/A | 3.0 | 2.0 |
| T41: 49 | 0.5 | Yes | 4.5 | 4.5 | 0.0 | IS | 3.0 | 2.0 |
| T41: 50 | 1.0 | Yes | 5.5 | 5.5 | 3.5 | N/A | 3.0 | 2.0 |
| T41: 51 | 0.8 | Yes | 6.5 | 6.5 | 4.5 | N/A | 3.0 | 2.0 |
| T41: 52 | 1.2 | Yes | 6.5 | 6.5 | 2.5 | N/A | 3.0 | 2.0 |
| T41: 53 | 0.5 | Yes | 5.5 | 5.5 | 5.5 | N/A | 3.0 | 2.0 |
| T41: 54 | 1.0 | Yes | 6.5 | 6.5 | 3.5 | N/A | 3.0 | 2.0 |
| T41: 55 | 0.2 | Yes | 4.5 | 4.5 | 0.0 | N/A | 3.0 | 2.0 |
| T41: 56 | 0.5 | Yes | 5.5 | 5.5 | 3.5 | N/A | 3.0 | 2.0 |
| T41: 57 | 0.5 | No | 6.5 | 6.5 | 2.5 | N/A | 3.0 | 2.0 |
| T41: 58 | 0.8 | No | 5.5 | 5.5 | 3.5 | N/A | 3.0 | 2.0 |
| T41: 59 | 0.2 | No | 3.5 | 3.5 | 2.5 | N/A | 3.0 | 2.0 |
| T41: 60 | 1.5 | Yes | 7.5 | 7.5 | 4.5 | N/A | 3.0 | 2.0 |
| T41: 61 | 0.2 | Yes | 2.5 | 2.5 | 0.0 | IS | 3.0 | 2.0 |
| T41: 62 | 1.0 | Yes | 6.5 | 6.5 | 2.5 | N/A | 3.0 | 2.0 |
| T41: 63 | 0.5 | Yes | 1.5 | 1.5 | 0.0 | IS | 3.0 | 2.0 |
| T41: 64 | 0.5 | Yes | 3.5 | 3.5 | 2.5 | N/A | 3.0 | 2.0 |
| T41: 65 | 0.0 | Yes | 2.5 | 2.5 | 2.5 | N/A | 3.0 | 2.0 |
| T41: 66 | 0.0 | Yes | 2.5 | 2.5 | 2.5 | N/A | 3.0 | 2.0 |
| T41: 67 | 0.2 | Yes | 3.5 | 3.5 | 1.5 | N/A | 3.0 | 2.0 |
| T41: 68 | 0.8 | Yes | 3.5 | 3.5 | 1.5 | N/A | 3.0 | 2.0 |
| T41: 69 | 0.0 | Yes | 3.5 | 3.5 | 2.5 | N/A | 3.0 | 2.0 |
| T41: 70 | 1.0 | No | 6.5 | 6.5 | 2.5 | N/A | 3.0 | 2.0 |
| T41: 71 | 0.2 | Yes | 4.5 | 4.5 | 3.5 | N/A | 3.0 | 2.0 |
| T41: 72 | 0.5 | Yes | 3.5 | 3.5 | 2.5 | N/A | 3.0 | 2.0 |
| T41: 73 | 1.2 | No | 5.5 | 5.5 | 3.5 | N/A | 3.0 | 2.0 |
| T41: 74 | 0.8 | Yes | 5.5 | 5.5 | 4.5 | N/A | 3.0 | 2.0 |
| T41: 75 | 0.5 | Yes | 5.5 | 5.5 | 2.5 | N/A | 3.0 | 2.0 |
| T41: 76 | 0.8 | No | 6.5 | 6.5 | 4.5 | N/A | 3.0 | 2.0 |
| T41: 77 | 1.0 | Yes | 5.5 | 5.5 | 3.5 | N/A | 3.0 | 2.0 |
| T41: 78 | 1.0 | Yes | 6.5 | 5.5 | 3.5 | N/A | 3.0 | 2.0 |
| T41: 79 | 1.0 | Yes | 5.5 | 5.5 | 3.5 | N/A | 3.0 | 2.0 |
| T41: 80 | 0.5 | Yes | 5.5 | 5.5 | 2.5 | N/A | 3.0 | 2.0 |
| T41: 81 | 0.5 | Yes | 6.5 | 6.5 | 4.5 | N/A | 3.0 | 2.0 |
| T41: 82 | 1.0 | No | 5.5 | 5.5 | 3.5 | N/A | 3.0 | 2.0 |
| T41: 83 | 1.0 | Yes | FO | FO | N/A | N/A | FO | FO |
| T41: 84 | 0.5 | Yes | 2.5 | 2.5 | 0.0 | N/A | 3.0 | 1.0 |
| T41: 85 | 0.2 | Yes | 5.5 | 4.5 | N/A | N/A | 3.0 | 2.0 |
| T41: 86 | 0.8 | Yes | 6.5 | 5.5 | 4.5 | N/A | 3.0 | 2.0 |
| T41: 87 | 1.8 | Yes | 6.5 | 6.5 | 6.5 | N/A | 3.0 | 2.0 |
| T41: 88 | 1.2 | Yes | 2.5 | 2.5 | 0.0 | N/A | 3.0 | 2.0 |
| T41: 89 | 1.5 | Yes | 5.5 | 5.5 | 4.5 | N/A | 3.0 | 2.0 |
| T41: 90 | 0.5 | Yes | 4.5 | 3.5 | 0.0 | N/A | 3.0 | 2.0 |
| T41: 91 | 1.5 | No | 5.5 | 4.5 | 2.5 | N/A | 3.0 | 2.0 |
| T41: 92 | 0.2 | Yes | 2.5 | 2.5 | 0.0 | N/A | 3.0 | 2.0 |
| T41: 93 | 1.5 | Yes | 5.5 | 4.5 | 0.0 | N/A | 3.0 | 2.0 |
| T41: 94 | 0.5 | No | 5.5 | 5.5 | 2.5 | N/A | 3.0 | 2.0 |
| T41: 95 | 0.8 | No | 2.5 | 2.5 | 0.0 | N/A | 3.0 | 2.0 |
| T41: 96 | 1.0 | No | 5.5 | 4.5 | 0.0 | N/A | 3.0 | 2.0 |
| T41: 97 | 1.8 | No | 6.5 | 5.5 | 3.5 | N/A | 3.0 | 2.0 |
| T41: 98 | 0.8 | Yes | 4.5 | 3.5 | 3.5 | N/A | 3.0 | 2.0 |
| T41: 99 | 1.0 | Yes | 6.5 | 6.5 | 4.5 | N/A | 3.0 | 2.0 |
| T41: 100 | 0.0 | N/A | 3.5 | 2.5 | 0.0 | IS | 3.0 | 2.0 |
| T41: 101 | 0.2 | Yes | 6.5 | 5.5 | 3.5 | N/A | 3.0 | 2.0 |
| T41: 102 | 1.2 | No | 6.5 | 6.5 | 4.5 | N/A | 3.0 | 2.0 |
| T41: 103 | 1.5 | No | 6.5 | 6.5 | 5.5 | N/A | 3.0 | 2.0 |
| T41: 104 | 0.2 | Yes | 5.5 | 4.5 | 2.5 | N/A | 3.0 | 2.0 |
| T41: 105 | 0.5 | Yes | 7.5 | 5.5 | 3.5 | N/A | 3.0 | 2.0 |
| T41: 106 | 0.2 | Yes | 2.5 | 0.0 | 0.0 | N/A | 3.0 | 2.0 |
| T41: 107 | 0.8 | No | 5.5 | 4.5 | 3.5 | N/A | 3.0 | 2.0 |
| T41: 108 | 0.8 | No | 3.5 | 3.5 | 3.5 | N/A | 3.0 | 2.0 |
| T41: 109 | 0.5 | No | 5.5 | 4.5 | 3.5 | N/A | 3.0 | 2.0 |
| T41: 110 | 1.2 | No | 7.5 | 7.5 | 5.5 | N/A | 3.0 | 2.0 |
| T41: 111 | 1.5 | No | 9.2 | 0.0 | 0.0 | IS | 3.0 | 2.0 |
| T41: 112 | 1.2 | No | 9.2 | 8.8 | 0.0 | SL | 3.0 | 2.0 |
| T41: 113 | 1.5 | No | 9.2 | 9.2 | 8.5 | N/A | 3.0 | 2.0 |
| T41: 114 | 1.0 | No | 4.5 | 3.5 | 0.0 | IS | 3.0 | 2.0 |
| T41: 115 | 1.2 | No | 8.5 | 7.5 | 2.5 | N/A | 3.0 | 2.0 |
| T41: 116 | 1.0 | No | 6.5 | 5.5 | 2.5 | N/A | 3.0 | 2.0 |
| T41: 117 | 0.8 | No | 7.2 | 0.0 | 0.0 | IS | 3.0 | 2.0 |
| T41: 118 | 1.5 | No | 6.5 | 6.5 | 5.5 | N/A | 3.0 | 2.0 |
| T41: 119 | 0.0 | No | 2.5 | 0.0 | 0.0 | N/A | 3.0 | 2.0 |
| T41: 120 | 1.2 | No | 9.2 | 8.8 | 6.5 | N/A | 3.0 | 2.0 |
| T41: 121 | 1.0 | No | 3.5 | 3.5 | 2.5 | N/A | 3.0 | 2.0 |
| T41: 122 | 0.8 | No | 8.2 | 7.5 | 3.5 | N/A | 3.0 | 2.0 |
| T41: 123 | 1.2 | No | 8.8 | 7.5 | 4.5 | N/A | 3.0 | 2.0 |
| T41: 124 | 1.2 | No | 8.5 | 7.5 | 5.5 | N/A | 3.0 | 2.0 |
| T41: 125 | 1.0 | No | 8.5 | 8.2 | 6.5 | N/A | 3.0 | 2.0 |
| T41: 126 | 0.0 | No | 3.5 | 3.5 | 3.5 | N/A | 3.0 | 2.0 |
| T41: 127 | 0.0 | No | 4.5 | 4.5 | 2.5 | N/A | 3.0 | 2.0 |
| T41: 128 | 1.0 | No | 9.2 | 8.5 | 6.5 | N/A | 3.0 | 2.0 |
| T41: 129 | 0.0 | Yes | 5.5 | 0.0 | 0.0 | IS | 3.0 | 2.0 |
| T41: 130 | 0.0 | No | 4.5 | 4.5 | 2.5 | N/A | 3.0 | 2.0 |
| T41: 131 | 0.0 | No | 7.5 | 5.5 | 3.5 | N/A | 3.0 | 2.0 |
| T41: 132 | 0.2 | No | 4.5 | 4.5 | 2.5 | N/A | 3.0 | 2.0 |
| T41: 133 | 1.0 | No | 7.5 | 7.5 | 4.5 | N/A | 3.0 | 2.0 |
| T41: 134 | 1.2 | No | 8.8 | 8.5 | 4.5 | N/A | 3.0 | 2.0 |
| T41: 135 | 0.2 | No | 6.5 | 5.5 | 2.5 | N/A | 3.0 | 3.0 |
| T41: 136 | 0.0 | No | 3.5 | 3.5 | 0.0 | N/A | 3.0 | 2.0 |
| T41: 137 | 1.0 | No | 7.5 | 7.5 | 7.5 | N/A | 3.0 | 3.0 |
| T41: 138 | 0.2 | No | 9.2 | 9.2 | 6.5 | N/A | 3.0 | 2.0 |
| T41: 139 | 0.5 | No | 0.0 | 0.0 | 0.0 | N/A | 3.0 | 2.0 |
| T41: 140 | 0.8 | No | 0.0 | 1.5 | N/A | N/A | 3.0 | 2.0 |
| T41: 141 | 0.0 | N/A | 8.8 | 8.5 | N/A | N/A | 3.0 | 2.0 |
| T41: 142 | 1.2 | No | 8.5 | 8.2 | 3.5 | N/A | 3.0 | 2.0 |
| T41: 143 | 0.2 | No | 8.2 | 7.5 | 4.5 | N/A | 3.0 | 2.0 |
| T41: 144 | 1.2 | No | 8.8 | 8.5 | N/A | N/A | 3.0 | 2.0 |
| T41: 145 | 1.2 | No | 9.2 | 9.2 | 0.0 | FT | 3.0 | 2.0 |
| T41: 146 | 1.2 | No | 9.5 | 9.2 | 4.0 | N/A | 3.0 | 2.0 |

Table 42 records the increases/decreases in C scores over time as a measure of the duration of the effect of ingesting a treatment substance on TSF, for some working examples listed in previous tables. These working examples measured TSF for C scores at varying time points after the first ingestion of a treatment substance and before the measurement of the B score. Negative time scores indicate that the C score measurement occurred prior to ingesting a treatment substance, such as at example T37:82 the C2 time is listed as −20 as the measurement for the C score at the C1 time was made 20 minutes before ingesting the treatment substance. In some examples additional rows were used to record C scores (e.g., examples T37:82 and T40:1). The duration of the effect of treatment substance(s) on the C score varied, with some reaching a peak C score before the B score measurement (e.g., see example T37:43), and other still increasing in C score to the B score measurement (e.g., see example T41:8).

TABLE 42
C Score Improvement Duration After Ingesting a Treatment Substance

| | C1 | C1 | C2 | C2 | C3 | C3 | C4 | C4 | C Score at | B Score |
|---|---|---|---|---|---|---|---|---|---|---|
| Example | Score | Time | Score | Time | Score | Time | Score | Time | B Score | Time |
| T36: 268 | N/A | N/A | 2.0 | 30 | N/A | N/A | N/A | N/A | 2.0 | 60 |
| T36: 270 | N/A | N/A | N/A | N/A | 2.0 | 40 | N/A | N/A | 1.8 | 60 |
| T36: 271 | N/A | N/A | N/A | N/A | N/A | N/A | 2.0 | 45 | 1.8 | 60 |
| T36: 282 | 1.8 | 10 | N/A | N/A | N/A | N/A | N/A | N/A | 1.0 | 60 |
| T37: 13 | N/A | N/A | 2.8 | 33 | N/A | N/A | N/A | N/A | 2.2 | 60 |
| T37: 15 | N/A | N/A | 2.5 | 30 | N/A | N/A | N/A | N/A | 2.0 | 60 |
| T37: 16 | N/A | N/A | 2.0 | 30 | N/A | N/A | N/A | N/A | 1.5 | 60 |
| T37: 17 | N/A | N/A | 2.0 | 35 | N/A | N/A | N/A | N/A | 1.8 | 60 |
| T37: 19 | N/A | N/A | 1.8 | 30 | N/A | N/A | N/A | N/A | 1.2 | 60 |
| T37: 20 | N/A | N/A | 2.2 | 30 | N/A | N/A | N/A | N/A | 1.5 | 60 |
| T37: 21 | N/A | N/A | 2.2 | 29 | 1.2 | 41 | N/A | N/A | 1.2 | 60 |
| T37: 24 | N/A | N/A | N/A | N/A | 2.2 | 40 | N/A | N/A | 2.2 | 60 |
| T37: 25 | N/A | N/A | N/A | N/A | 2.0 | 35 | N/A | N/A | 1.8 | 60 |
| T37: 26 | N/A | N/A | 1.8 | 30 | N/A | N/A | N/A | N/A | 1.8 | 60 |
| T37: 34 | N/A | N/A | N/A | N/A | 2.0 | 40 | N/A | N/A | 2.0 | 60 |
| T37: 36 | N/A | N/A | N/A | N/A | 2.2 | 40 | N/A | N/A | 1.8 | 60 |
| T37: 39 | N/A | N/A | N/A | N/A | N/A | N/A | 2.0 | 45 | 1.8 | 60 |
| T37: 41 | N/A | N/A | 2.2 | 30 | N/A | N/A | N/A | N/A | 1.8 | 60 |
| T37: 42 | N/A | N/A | N/A | N/A | 1.8 | 40 | N/A | N/A | 1.8 | 60 |
| T37: 43 | 2.2 | 40 | 2.8 | 45 | 3.2 | 50 | 2.2 | 55 | 1.8 | 60 |
| T37: 44 | N/A | N/A | N/A | N/A | 2.0 | 40 | N/A | N/A | 1.2 | 60 |
| T37: 45 | N/A | N/A | N/A | N/A | 1.8 | 40 | N/A | N/A | 2.0 | 60 |
| T37: 46 | N/A | N/A | N/A | N/A | 2.0 | 41 | N/A | N/A | 1.2 | 60 |
| T37: 48 | N/A | N/A | N/A | N/A | 2.0 | 40 | N/A | N/A | 1.2 | 60 |
| T37: 49 | N/A | N/A | N/A | N/A | 2.2 | 40 | N/A | N/A | 2.2 | 60 |
| T37: 51 | N/A | N/A | N/A | N/A | 1.8 | 40 | N/A | N/A | 1.5 | 60 |
| T37: 54 | N/A | N/A | N/A | N/A | 2.0 | 40 | N/A | N/A | 1.8 | 60 |
| T37: 58 | N/A | N/A | N/A | N/A | 1.8 | 40 | N/A | N/A | 1.5 | 60 |
| T37: 59 | N/A | N/A | N/A | N/A | 1.8 | 40 | N/A | N/A | 1.5 | 60 |
| T37: 60 | N/A | N/A | N/A | N/A | 1.8 | 40 | N/A | N/A | 1.5 | 60 |
| T37: 61 | N/A | N/A | N/A | N/A | 1.8 | 40 | N/A | N/A | 1.2 | 60 |
| T37: 62 | N/A | N/A | N/A | N/A | 2.0 | 35 | N/A | N/A | 1.2 | 60 |
| T37: 63 | N/A | N/A | N/A | N/A | 1.5 | 40 | N/A | N/A | 1.0 | 60 |
| T37: 64 | N/A | N/A | N/A | N/A | 1.8 | 40 | N/A | N/A | 1.5 | 60 |
| T37: 69 | N/A | N/A | N/A | N/A | N/A | N/A | 4.0 | 225 | 5.0 | 60 |
| T37: 72 | N/A | N/A | 2.5 | 30 | N/A | N/A | N/A | N/A | 2.0 | 60 |
| T37: 75 | N/A | N/A | N/A | N/A | 2.0 | 40 | N/A | N/A | 1.8 | 60 |
| T37: 76 | N/A | N/A | N/A | N/A | 2.0 | 40 | 2.0 | 50 | 2.0 | 60 |
| T37: 77 | N/A | N/A | N/A | N/A | 2.0 | 40 | N/A | N/A | 1.0 | 60 |
| T37: 80 | N/A | N/A | 1.5 | 34 | 1.0 | 40 | N/A | N/A | 0.2 | 60 |
| T37: 81 | N/A | N/A | 1.5 | 25 | 1.0 | 47 | 0.5 | 52 | 0.5 | 60 |
| T37: 82* | 0.0 | −25 | 1.0 | 15 | 1.5 | 20 | 1.0 | 30 | 0.2 | 60 |
| | N/A | N/A | N/A | N/A | N/A | N/A | 0.5 | 50 | | |
| T37: 83 | 1.5 | 25 | 1.2 | 30 | N/A | N/A | 1.2 | 45 | 1.2 | 60 |
| T37: 84 | 1.2 | 15 | N/A | N/A | N/A | N/A | N/A | N/A | 0.5 | 60 |
| T37: 87 | N/A | N/A | N/A | N/A | 2.0 | 40 | N/A | N/A | 1.2 | 60 |
| T38: 16 | 0.8 | 23 | N/A | N/A | N/A | N/A | N/A | N/A | 0.2 | 60 |
| T39: 4 | N/A | N/A | N/A | N/A | N/A | N/A | 2.0 | 65 | 1.2 | 80 |
| T39: 12 | N/A | N/A | N/A | N/A | N/A | N/A | 2.0 | 40 | 1.8 | 45 |
| T39: 13 | 2.0 | 20 | N/A | N/A | N/A | N/A | N/A | N/A | 1.5 | 40 |
| T39: 14A | 0.2 | −5 | 0.8 | 0 | 1.5 | 10 | 1.5 | 20 | 1.2 | 30 |
| T39: 14B | N/A | N/A | N/A | N/A | N/A | N/A | N/A | N/A | 1.0 | 70 |
| T39: 15 | 0.0 | −20 | 1.2 | 5 | 1.5 | 10 | 2.0 | 20 | 2.0 | 30 |
| T39: 16 | 0.5 | −10 | 0.5 | 0 | 1.2 | 10 | 2.0 | 20 | 2.0 | 30 |
| T40: 1* | 2.0 | 35 | 2.0 | 40 | 2.0 | 45 | 2.0 | 50 | 2.0 | 70 |
| | N/A | N/A | 2.0 | 55 | 2.0 | 60 | 2.0 | 65 | | |
| T40: 3A | N/A | N/A | N/A | N/A | N/A | N/A | 4.0 | 59 | 3.0 | 79 |
| T40: 3B | N/A | N/A | N/A | N/A | N/A | N/A | N/A | N/A | 2.0 | 139 |
| T40: 4A | N/A | N/A | N/A | N/A | N/A | N/A | N/A | N/A | 1.5 | 60 |
| T40: 4B | N/A | N/A | N/A | N/A | N/A | N/A | N/A | N/A | 1.5 | 192 |
| T40: 6 | N/A | N/A | N/A | N/A | 3.0 | 60 | 2.8 | 70 | 2.5 | 80 |
| T40: 7 | N/A | N/A | 2.0 | 50 | 2.0 | 60 | 1.8 | 70 | 1.5 | 85 |
| T40: 8 | N/A | N/A | 2.0 | 35 | N/A | N/A | N/A | N/A | 2.5 | 71 |
| T40: 10 | N/A | N/A | 1.2 | 25 | 1.8 | 40 | N/A | N/A | 1.8 | 60 |
| T40: 11 | N/A | N/A | N/A | N/A | N/A | N/A | 2.0 | 60 | 1.2 | 120 |
| T40: 13 | 0.0 | −20 | N/A | N/A | N/A | N/A | 1.8 | 50 | 1.2 | 60 |
| T41: 1 | N/A | N/A | 1.8 | 30 | 1.2 | 40 | 1.0 | 50 | 1.0 | 60 |
| T41: 2 | N/A | N/A | 1.0 | 30 | 1.8 | 40 | 1.2 | 50 | 0.0 | 60 |
| T41: 3 | N/A | N/A | 0.0 | 30 | 0.0 | 40 | 0.0 | 50 | 0.0 | 60 |
| T41: 4 | N/A | N/A | 1.2 | 30 | 1.2 | 40 | 0.5 | 50 | 0.0 | 60 |
| T41: 5 | N/A | N/A | 0.0 | 30 | 0.0 | 40 | 0.0 | 50 | 0.0 | 60 |
| T41: 6 | N/A | N/A | 0.0 | 30 | 0.5 | 40 | 0.5 | 50 | 0.0 | 60 |
| T41: 7 | N/A | N/A | 0.2 | 30 | 0.5 | 40 | 0.8 | 50 | 1.0 | 60 |
| T41: 8 | N/A | N/A | 0.8 | 30 | 1.2 | 40 | 1.2 | 50 | 1.5 | 60 |
| T41: 9 | N/A | N/A | 0.5 | 30 | 0.8 | 40 | 0.2 | 50 | 0.0 | 60 |
| T41: 10 | N/A | N/A | 1.5 | 30 | 1.2 | 40 | 1.2 | 50 | 1.0 | 60 |
| T41: 11 | N/A | N/A | 0.2 | 30 | 0.2 | 40 | 0.5 | 50 | 0.2 | 60 |
| T41: 12 | N/A | N/A | 0.5 | 30 | 0.2 | 40 | 0.0 | 50 | 0.0 | 60 |
| T41: 13 | N/A | N/A | 1.0 | 30 | 0.8 | 40 | 1.0 | 50 | 0.5 | 60 |
| T41: 14 | N/A | N/A | 0.5 | 30 | 1.0 | 40 | 1.0 | 50 | 1.0 | 60 |
| T41: 15 | N/A | N/A | 0.0 | 30 | 1.0 | 40 | 0.5 | 50 | 0.0 | 60 |
| T41: 16 | N/A | N/A | 0.0 | 30 | 0.0 | 40 | 0.0 | 50 | 0.2 | 60 |
| T41: 17 | N/A | N/A | 0.2 | 30 | 0.2 | 40 | 0.2 | 50 | 0.2 | 60 |
| T41: 18 | N/A | N/A | 0.0 | 30 | 0.5 | 40 | 0.5 | 50 | 0.2 | 60 |
| T41: 19 | N/A | N/A | 0.0 | 30 | 0.0 | 40 | 0.0 | 50 | 0.2 | 60 |
| T41: 20 | N/A | N/A | 0.0 | 30 | 0.0 | 40 | 0.2 | 50 | 0.2 | 60 |
| T41: 21 | N/A | N/A | 0.8 | 30 | 0.0 | 40 | 0.0 | 50 | 0.0 | 60 |
| T41: 22 | N/A | N/A | 0.0 | 30 | 1.0 | 40 | 0.5 | 50 | 0.5 | 60 |
| T41: 23 | N/A | N/A | 1.8 | 30 | 1.8 | 40 | 1.5 | 50 | 1.2 | 60 |
| T41: 24 | N/A | N/A | 0.5 | 30 | 0.8 | 40 | 0.8 | 50 | 0.5 | 60 |
| T41: 25 | N/A | N/A | 0.2 | 30 | 0.2 | 40 | 0.2 | 50 | 0.2 | 60 |
| T41: 26 | N/A | N/A | 0.2 | 30 | 0.5 | 40 | 1.0 | 50 | 0.2 | 60 |
| T41: 27 | N/A | N/A | 0.2 | 30 | 0.2 | 40 | 0.5 | 50 | 0.5 | 60 |
| T41: 28 | N/A | N/A | 0.5 | 30 | 0.5 | 40 | 0.5 | 50 | 0.5 | 60 |
| T41: 29 | N/A | N/A | 1.2 | 30 | 1.2 | 40 | 1.0 | 50 | 0.8 | 60 |
| T41: 30 | N/A | N/A | 0.8 | 30 | 1.0 | 40 | 1.2 | 50 | 1.2 | 60 |
| T41: 31 | N/A | N/A | 1.2 | 30 | 1.2 | 40 | 1.2 | 50 | 1.0 | 60 |
| T41: 32 | N/A | N/A | 1.5 | 30 | 1.5 | 40 | 1.0 | 50 | 1.0 | 60 |
| T41: 33 | N/A | N/A | 0.2 | 30 | 0.5 | 40 | 1.0 | 50 | 1.0 | 60 |
| T41: 34 | N/A | N/A | 1.0 | 30 | 0.5 | 40 | 0.5 | 50 | 0.5 | 60 |
| T41: 35 | N/A | N/A | 0.2 | 30 | 0.2 | 40 | 1.0 | 50 | 1.0 | 60 |
| T41: 36 | N/A | N/A | 0.5 | 30 | 0.5 | 40 | 0.5 | 50 | 0.5 | 60 |
| T41: 37 | N/A | N/A | 1.2 | 30 | 1.5 | 40 | 1.2 | 50 | 1.0 | 60 |
| T41: 38 | N/A | N/A | 0.0 | 30 | 0.0 | 40 | 0.5 | 50 | 0.5 | 60 |
| T41: 39 | N/A | N/A | 0.2 | 30 | 0.5 | 40 | 0.8 | 50 | 0.5 | 60 |
| T41: 40 | N/A | N/A | 1.0 | 30 | 0.8 | 40 | 0.5 | 50 | 0.2 | 60 |
| T41: 41 | N/A | N/A | 0.0 | 30 | 0.0 | 40 | 0.2 | 50 | 0.2 | 60 |
| T41: 42 | 1.5 | 20 | 1.0 | 30 | 1.0 | 40 | 1.0 | 50 | 0.5 | 60 |
| T41: 43 | N/A | N/A | 1.5 | 30 | 1.2 | 40 | 1.2 | 50 | 1.2 | 60 |
| T41: 44 | 1.5 | 20 | 1.5 | 30 | 1.2 | 40 | 1.2 | 50 | 1.0 | 60 |
| T41: 45 | 0.5 | 20 | 1.2 | 30 | 0.5 | 40 | 0.2 | 50 | 0.0 | 60 |
| T41: 46 | 0.8 | 20 | 1.0 | 30 | 1.2 | 40 | 0.8 | 50 | 0.2 | 60 |
| T41: 47 | N/A | N/A | 0.5 | 30 | 0.5 | 40 | 0.5 | 50 | 0.5 | 60 |
| T41: 48 | N/A | N/A | 0.2 | 30 | 0.2 | 40 | 0.5 | 50 | 0.2 | 60 |
| T41: 49 | 0.5 | 20 | 0.5 | 30 | 0.5 | 40 | 0.5 | 50 | 0.5 | 60 |
| T41: 50 | 0.5 | 20 | 0.5 | 30 | 1.0 | 40 | 1.0 | 50 | 1.0 | 60 |
| T41: 51 | 0.2 | 20 | 0.2 | 30 | 0.5 | 40 | 0.8 | 50 | 0.8 | 60 |
| T41: 52 | 1.0 | 20 | 1.5 | 30 | 1.5 | 40 | 1.2 | 50 | 1.2 | 60 |
| T41: 53 | 0.2 | 20 | 0.5 | 30 | 0.8 | 40 | 0.5 | 50 | 0.5 | 60 |
| T41: 54 | 0.2 | 20 | 0.5 | 30 | 0.5 | 40 | 0.8 | 50 | 1.0 | 60 |
| T41: 55 | 0.2 | 20 | 0.2 | 30 | 0.2 | 40 | 0.2 | 50 | 0.2 | 60 |
| T41: 56 | 0.5 | 20 | 0.5 | 30 | 1.0 | 40 | 0.8 | 50 | 0.5 | 60 |
| T41: 57 | 0.0 | 20 | 0.5 | 30 | 1.0 | 40 | 0.8 | 50 | 0.5 | 60 |
| T41: 58 | 0.2 | 20 | 0.5 | 30 | 1.0 | 40 | 0.8 | 50 | 0.8 | 60 |
| T41: 59 | 0.5 | 20 | 1.2 | 30 | 1.0 | 40 | 0.8 | 50 | 0.2 | 60 |
| T41: 60 | 0.8 | 20 | 1.5 | 30 | 1.8 | 40 | 1.8 | 50 | 1.5 | 60 |
| T41: 61 | 0.5 | 20 | 0.5 | 30 | 0.5 | 40 | 0.2 | 50 | 0.2 | 60 |
| T41: 62 | 1.2 | 20 | 1.2 | 30 | 1.2 | 40 | 1.2 | 50 | 1.0 | 60 |
| T41: 63 | 0.5 | 20 | 0.8 | 30 | 0.5 | 40 | 0.5 | 50 | 0.5 | 60 |
| T41: 64 | 1.0 | 20 | 0.8 | 30 | 1.0 | 40 | 0.8 | 50 | 0.5 | 60 |
| T41: 65 | 0.5 | 20 | 1.0 | 30 | 0.5 | 40 | 0.2 | 50 | 0.0 | 60 |
| T41: 66 | 0.2 | 20 | 0.5 | 30 | 0.2 | 40 | 0.2 | 50 | 0.0 | 60 |
| T41: 67 | 0.2 | 20 | 0.2 | 30 | 0.0 | 40 | 0.2 | 50 | 0.2 | 60 |
| T41: 68 | 0.2 | 20 | 0.5 | 30 | 0.5 | 40 | 0.8 | 50 | 0.8 | 60 |
| T41: 69 | 0.2 | 20 | 0.0 | 30 | 0.2 | 40 | 0.2 | 50 | 0.0 | 60 |
| T41: 70 | 0.5 | 20 | 0.5 | 30 | 1.0 | 40 | 1.2 | 50 | 1.0 | 60 |
| T41: 71 | 0.0 | 20 | 0.5 | 30 | 0.5 | 40 | 0.2 | 50 | 0.2 | 60 |
| T41: 72 | 0.0 | 20 | 0.2 | 30 | 0.5 | 40 | 0.5 | 50 | 0.5 | 60 |
| T41: 73 | 0.5 | 20 | 1.0 | 30 | 1.0 | 40 | 1.2 | 50 | 1.2 | 60 |
| T41: 74 | 0.8 | 20 | 0.5 | 30 | 0.8 | 40 | 0.5 | 50 | 0.8 | 60 |
| T41: 75 | 0.5 | 20 | 0.5 | 30 | 0.5 | 40 | 0.8 | 50 | 0.5 | 60 |
| T41: 76 | 1.0 | 20 | 1.2 | 30 | 1.0 | 40 | 0.8 | 50 | 0.8 | 60 |
| T41: 77 | 0.8 | 20 | 0.8 | 30 | 1.0 | 40 | 1.2 | 50 | 1.0 | 60 |
| T41: 78 | 0.8 | 20 | 1.0 | 30 | 1.0 | 40 | 1.2 | 50 | 1.0 | 60 |
| T41: 79 | 0.8 | 20 | 0.5 | 30 | 1.0 | 40 | 1.0 | 50 | 1.0 | 60 |
| T41: 80 | 0.8 | 20 | 0.5 | 30 | 0.5 | 40 | 0.5 | 50 | 0.5 | 60 |
| T41: 81 | 0.2 | 20 | 0.2 | 30 | 0.5 | 40 | 0.8 | 50 | 0.5 | 60 |
| T41: 82 | 1.0 | 20 | 1.2 | 30 | 1.2 | 40 | 1.0 | 50 | 1.0 | 60 |
| T41: 83 | 0.2 | 20 | 0.5 | 30 | 0.5 | 40 | 0.8 | 50 | 1.0 | 60 |
| T41: 84 | 0.8 | 20 | 0.8 | 30 | 1.0 | 40 | 1.2 | 50 | 0.5 | 60 |
| T41: 85 | 0.2 | 20 | 0.5 | 30 | 0.5 | 40 | 0.8 | 50 | 0.2 | 60 |
| T41: 86 | 0.5 | 20 | 0.8 | 30 | 0.5 | 40 | 0.8 | 50 | 0.8 | 60 |
| T41: 87 | 1.5 | 20 | 2.0 | 30 | 2.0 | 40 | 1.8 | 50 | 1.8 | 60 |
| T41: 88 | 0.2 | 20 | 0.5 | 30 | 1.5 | 40 | 1.5 | 50 | 1.2 | 60 |
| T41: 89 | 0.5 | 20 | 0.8 | 30 | 1.5 | 40 | 1.5 | 50 | 1.5 | 60 |
| T41: 90 | 1.0 | 20 | 1.0 | 30 | 0.8 | 40 | 0.5 | 50 | 0.5 | 60 |
| T41: 91 | 1.0 | 20 | 1.2 | 30 | 1.5 | 40 | 1.8 | 50 | 1.5 | 60 |
| T41: 92 | 1.0 | 20 | 0.8 | 30 | 1.0 | 40 | 0.5 | 50 | 0.2 | 60 |
| T41: 93 | 0.5 | 20 | 1.0 | 30 | 1.0 | 40 | 1.2 | 50 | 1.5 | 60 |
| T41: 94 | 0.2 | 20 | 1.0 | 30 | 1.5 | 40 | 1.0 | 50 | 0.5 | 60 |
| T41: 95 | 0.2 | 20 | 1.2 | 30 | 1.5 | 40 | 1.2 | 50 | 0.8 | 60 |
| T41: 96 | 0.5 | 20 | 0.8 | 30 | 1.0 | 40 | 1.0 | 50 | 1.0 | 60 |
| T41: 97 | 0.5 | 20 | 0.8 | 30 | 1.2 | 40 | 1.5 | 50 | 1.8 | 60 |
| T41: 98 | 0.2 | 20 | 0.2 | 30 | 0.5 | 40 | 1.0 | 50 | 0.8 | 60 |
| T41: 99 | 0.5 | 20 | 1.5 | 30 | 1.2 | 40 | 1.2 | 50 | 1.0 | 60 |
| T41: 100 | 0.0 | 20 | 0.0 | 30 | 0.0 | 40 | 0.0 | 50 | 0.0 | 60 |
| T41: 101 | 0.2 | 20 | 0.2 | 30 | 0.0 | 40 | 0.2 | 50 | 0.2 | 60 |
| T41: 102 | 1.0 | 20 | 1.8 | 30 | 1.2 | 40 | 1.5 | 50 | 1.2 | 60 |
| T41: 103 | 1.8 | 20 | 1.8 | 30 | 1.2 | 40 | 1.5 | 50 | 1.5 | 60 |
| T41: 104 | 1.2 | 20 | 1.2 | 30 | 1.2 | 40 | 0.2 | 50 | 0.2 | 60 |
| T41: 105 | 2.0 | 20 | 1.8 | 30 | 2.0 | 40 | 1.0 | 50 | 0.5 | 60 |
| T41: 106 | 1.2 | 20 | 1.2 | 30 | 0.8 | 40 | 0.2 | 50 | 0.2 | 60 |
| T41: 107 | 0.8 | 20 | 0.8 | 30 | 0.8 | 40 | 1.0 | 50 | 0.8 | 60 |
| T41: 108 | 0.0 | 20 | 0.5 | 30 | 1.0 | 40 | 0.8 | 50 | 0.8 | 60 |
| T41: 109 | 0.5 | 20 | 0.8 | 30 | 1.2 | 40 | 1.0 | 50 | 0.5 | 60 |
| T41: 110 | 0.2 | 20 | 1.5 | 30 | 1.2 | 40 | 1.0 | 50 | 1.2 | 60 |
| T41: 111 | 0.5 | 20 | 1.0 | 30 | 1.5 | 40 | 1.5 | 50 | 1.5 | 60 |
| T41: 112 | 1.2 | 20 | 1.8 | 30 | 2.0 | 40 | 1.2 | 50 | 1.2 | 60 |
| T41: 113 | 1.2 | 20 | 1.5 | 30 | 2.0 | 40 | 2.0 | 50 | 1.5 | 60 |
| T41: 114 | 1.2 | 20 | 1.8 | 30 | 2.2 | 40 | 2.0 | 50 | 1.0 | 60 |
| T41: 115 | 1.2 | 20 | 1.5 | 30 | 1.8 | 40 | 1.5 | 50 | 1.2 | 60 |
| T41: 116 | 0.8 | 20 | 1.5 | 30 | 1.5 | 40 | 1.2 | 50 | 1.0 | 60 |
| T41: 117 | 1.0 | 20 | 1.0 | 30 | 1.0 | 40 | 1.2 | 50 | 0.8 | 60 |
| T41: 118 | 0.5 | 20 | 1.2 | 30 | 1.2 | 40 | 1.2 | 50 | 1.5 | 60 |
| T41: 119 | 0.8 | 20 | 0.0 | 30 | 0.0 | 40 | 0.2 | 50 | 0.0 | 60 |
| T41: 120 | 2.0 | 20 | 1.2 | 30 | 1.0 | 40 | 1.2 | 50 | 1.2 | 60 |
| T41: 121 | 1.0 | 20 | 1.2 | 30 | 1.5 | 40 | 1.5 | 50 | 1.0 | 60 |
| T41: 122 | 1.8 | 20 | 1.5 | 30 | 1.2 | 40 | 1.2 | 50 | 0.8 | 60 |
| T41: 123 | 0.8 | 20 | 1.8 | 30 | 1.5 | 40 | 1.5 | 50 | 1.2 | 60 |
| T41: 124 | 1.2 | 20 | 1.5 | 30 | 1.5 | 40 | 1.2 | 50 | 1.2 | 60 |
| T41: 125 | 1.2 | 20 | 1.2 | 30 | 1.5 | 40 | 1.5 | 50 | 1.0 | 60 |
| T41: 126 | 0.8 | 20 | 0.8 | 30 | 0.5 | 40 | 0.2 | 50 | 0.0 | 60 |
| T41: 127 | 1.2 | 20 | 1.2 | 30 | 1.0 | 40 | 0.5 | 50 | 0.0 | 60 |
| T41: 128 | 1.0 | 20 | 1.8 | 30 | 0.8 | 40 | 1.0 | 50 | 1.0 | 60 |
| T41: 129 | 0.5 | 20 | 0.8 | 30 | 0.2 | 40 | 0.0 | 50 | 0.0 | 60 |
| T41: 130 | 1.0 | 20 | 0.5 | 30 | 0.0 | 40 | 0.2 | 50 | 0.0 | 60 |
| T41: 131 | 1.5 | 20 | 1.2 | 30 | 1.0 | 40 | 0.8 | 50 | 0.0 | 60 |
| T41: 132 | 0.5 | 20 | 0.5 | 30 | 0.8 | 40 | 0.2 | 50 | 0.2 | 60 |
| T41: 133 | 0.5 | 20 | 1.0 | 30 | 1.0 | 40 | 1.0 | 50 | 1.0 | 60 |
| T41: 134 | 1.2 | 20 | 1.2 | 30 | 1.5 | 40 | 1.2 | 50 | 1.2 | 60 |
| T41: 135 | 1.8 | 20 | 0.0 | 30 | 0.0 | 40 | 0.5 | 50 | 0.2 | 60 |
| T41: 136 | 1.2 | 20 | 0.8 | 30 | 0.2 | 40 | 0.0 | 50 | 0.0 | 60 |
| T41: 137 | 1.5 | 20 | 1.5 | 30 | 1.0 | 40 | 1.0 | 50 | 1.0 | 60 |
| T41: 138 | 1.0 | 20 | 1.5 | 30 | 1.5 | 40 | 0.5 | 50 | 0.2 | 60 |
| T41: 139 | 0.5 | 20 | 0.5 | 30 | 1.2 | 40 | 0.5 | 50 | 0.5 | 60 |
| T41: 140 | N/A | N/A | N/A | N/A | 3.0 | 40 | 1.5 | 50 | 0.8 | 60 |
| T41: 142 | 1.5 | 20 | 2.0 | 30 | 1.8 | 40 | 1.2 | 50 | 1.2 | 60 |
| T41: 143 | 2.0 | 20 | 1.2 | 30 | 0.5 | 40 | 0.2 | 50 | 0.2 | 60 |
| T41: 144* | 0.0 | −30 | 0.2 | 5 | 1.0 | 20 | 1.0 | 30 | 1.2 | 60 |
| | N/A | N/A | N/A | N/A | 1.5 | 40 | 1.5 | 50 | | |
| T41: 145* | 0.8 | −9 | 0.8 | 0 | 1.2 | 10 | 1.5 | 20 | 1.2 | 60 |
| | N/A | N/A | 1.5 | 30 | 1.2 | 40 | 1.2 | 50 | | |
| T41: 146* | 0.2 | −30 | 0.2 | 0 | 1.0 | 10 | 1.8 | 20 | 1.2 | 60 |
| | N/A | N/A | 1.8 | 30 | 1.2 | 40 | 1.5 | 50 | | |

*Row Added to List Additional C Scores and Times of Measurements

Most working examples had no noticeable non-sexual side effects associated with ingesting a treatment substance. In some examples a mild change from feeling normal occurred, such as the feeling of being under the influence of a medication, slightly sleepy, slightly mentally unfocused, etc., but these alterations were not inhibitory of normal function, and are referred to herein as “felt not normal.” The time (which was sometimes approximated) and type of a change that may be a non-sexual side effect due to ingesting treatment substances are shown at Table 43.

TABLE 43
Non-Sexual Side Effects Associated with Treatment Substances

| | Time of | | Treatment Substance (Notable |
|---|---|---|---|
| | Side Effect | | Treatment Substance Associated with |
| Example | (Minutes) | Side Effect | Side Effect) |
| T36: 257 | 60 | Felt flushed and agitated | Val, MagT, NAC.SR, Gly, (Yohimbe |
| | | | Bark) |
| T36: 258 | 60 | Felt grumpy | NGABA, MagT, NAC.SR, Gly, Lemon |
| | | | Balm |
| T36: 259 | 60 | Felt warm feeling on skin in | NGABA, MagT, NAC.SR, Gly, Grape |
| | | general | SeedR, Citrus Bioflavonoids |
| T36: 260 | 60 | Felt grumpy | NGABA, MagT, NAC.SR, Gly, |
| | | | R-Alpha-Lipoic Acid |
| T36: 261 | 30 | Felt hot, flushed, and agitated | NGABA, MagT, NAC.SR, Gly, |
| | | | (Yohimbe Bark) |
| T36: 262 | 30 | Felt prickly sensation on skin | NGABA, GABA |
| | | in general | |
| T36: 263 | 60 | Felt positive, laughed easily | NGABA, Alpha-Lipoic Acid |
| T36: 264 | 60-360 | At 60 minutes: felt not | NGABA, (Yohimbe Bark) |
| | | normal, grumpy, and | |
| | | agitated; about 360 minutes: | |
| | | had insomnia | |
| T36: 266 | 20 | Face and arms tingled | NGABA, Beta-Ala |
| T36: 267 | 65 | Reduced interest in drinking | Inosine |
| | | wine | |
| T36: 268 | 60 to 130 | At 60 minutes: heart rate | Val, MagT, NAC.P, Galantamine HBr |
| | | seemed slower; alcohol | |
| | | drank at 65 minutes: felt not | |
| | | normal with feeling peaking | |
| | | at 90 minutes; at 130 | |
| | | minutes: felt normal after | |
| | | eating food | |
| T36: 269 | 50 | Felt grumpy, agitated, and | Val, (Yohimbe Bark) |
| | | flushed with rapid heartbeat | |
| T36: 270 | 90 | Wine ingested at 75 minutes, | Val, MagT, NAC.P, NAC.SR, |
| | | at 90 minutes: felt not normal | Galantamine HBr |
| T36: 271 | 30-60 | Felt not normal | Val, MagT, NAC.P, D-Ser, DimethylGly, |
| | | | Galantamine HBr |
| T36: 272 | 30 | Face tingled, warm taste in | Val, GABA |
| | | mouth | |
| T36: 275 | 60 | Felt not normal | Sarcosine |
| T36: 277 | 60 | Mild headache during A and | Val, MagT, NAC.P, Saffron Extract, |
| | | B scores | Huperzine A, Kava, Lemon Balm |
| T36: 278 | 10-15 | Felt not normal, then felt | Sarpagandha |
| | | normal, then felt not normal | |
| | | and became impulsive and | |
| | | danced | |
| T37: 6 | 60 | Felt agitated | NGABA, MagT, NAC.SR, Gly, Theanine |
| T37: 10 | 60 | Felt not normal | Val, MagT, NAC.SR, Gly, Tyr |
| T37: 25 | 20 | Felt not normal | Val, MagT, NAC.P |
| T37: 26 | 0 | Stomach discomfort | Val, NAC.P, Cys |
| T37: 30 | 15 | Felt not normal | Val, MagT, NAC.P, Gly, AKG |
| T37: 36 | 40-190 | At 40 minutes: felt not | Val, MagT, NAC.P, D-Ser, DimethylGly |
| | | normal; at 190 minutes: | |
| | | laughed easily, agitated | |
| T37: 37 | 15 | Stomach discomfort | Val, MagT, NAC.P, Galantamine HBr |
| T37: 39 | 65 | Felt not normal | NGABA, MagT, NAC.P, Galantamine |
| | | | HBr |
| T37: 40 | 65 | Felt not normal, agitated, | Val, NAC.SR, NAC.P, Galantamine HBr |
| | | more socially assertive, and | |
| | | grumpy | |
| T37: 41 | 65 | Felt not normal, agitated | Val, NAC.SR, NAC.P, Galantamine HBr, |
| | | | MagT |
| T37: 42 | 15 | Stomach discomfort | NGABA, MagT, NAC.P, D-Ser, |
| | | | DimethylGly, Galantamine HBr |
| T37: 44 | 198 | Felt positive | NGABA, NAC.P, |
| | | | Alpha-GlyceroPhosphoCholine, |
| | | | DimethylGly, Galantamine HBr |
| T37: 54 | 150 | Vomited | (Homotaurine), D-Ser |
| T37: 56 | 150 | Vomited | (Homotaurine) |
| T37: 57 | 120 | Nausea | (Homotaurine) |
| T37: 68 | 60 | Felt not normal | Val, NAC.P, MagT |
| T37: 74 | 30 | Felt agitated | Val, MagT, NAC.P, Saffron Extract, |
| | | | Huperzine A, Kava, Caffeine |
| T37: 75 | 10-50 | At 10 minutes: face, mouth, | NGABA, MagT, NAC.P, Saffron Extract, |
| | | hands, ears tingled; at 40-50 | Huperzine A, Kava, Beta-Ala |
| | | minutes: chest and arms | |
| | | tingled | |
| T37: 76 | 30-45 | Felt agitated | NGABA, MagT, NAC.P, Huperzine A, |
| | | | Saffron Extract, Lemon Balm, Caffeine |
| T37: 77 | 20 | Face, hands and ears tingled | Val, MagT, NAC.P, Saffron Extract, |
| | | | Huperzine A, Kava, Beta-Ala |
| T37: 80 | 12 | Stomach discomfort | Pramiracetam |
| T38: 6 | 60 | Felt not normal | Sarcosine |
| T39: 13 | 20-120 | Felt not normal | Phenibut |
| T40: 8 | 71 | Felt not normal | Val, NAC.P |
| T41: 6 | 30 | Mild nausea | (Lobelia) |
| T41: 23 | 40-60 | Felt not normal | Prickly Ash |
| T41: 28 | 30-50 | Mild nausea | (Lobelia) |
| T41: 37 | 5 | Stomach discomfort; small | Chamomile |
| | | amount of vomiting | |
| T41: 62 | 30-96 | At 30 to 60 minutes a slight | 4-DMA-7,8-DHF |
| | | headache and felt not normal; | |
| | | at 96 minutes: felt not normal | |
| | | peaked | |
| T41: 68 | 50-60 | Felt not normal | Belladonna |
| T41: 72 | 50-60 | Mild headache | BDNF |
| T41: 73 | 40-60 | Felt not normal | (98% Phloretin) |
| T41: 74 | 20-60 | Felt not normal | (98% Phloretin) |
| T41: 77 | 40-60 | Felt not normal | Red Clover |
| T41: 87 | 30-180 | At 30 minutes: felt not | (99% Rauwolscine) |
| | | normal; at 50 minutes: slight | |
| | | hand jitters and felt slight | |
| | | chill; side effects lasted over | |
| | | 180 minutes | |
| T41: 91 | 50-60 | Felt not normal | 80% Luteolin |
| T41: 97 | 50-60 | Felt not normal; nausea | Turkey Rhubarb |
| T41: 137 | 20-50 | Felt not normal | Val, NAC.P, D-Beta-Hydroxybutyrate |
| | | | Ester, Potassium |

Most working examples had no noticeable sexual effects other than those previously described for the measurements of the C, CT, A, B, BM, BS, D, DE scores, etc. A slightly different than normal sexual sensation for the C score being recorded as “C score different” and a sensitivity in the urethra typically occurring when urinating after sexual stimulation before SRSS began being recorded as “urination sensitivity.” The ease to ejaculate and/or orgasm was sometimes promoted or reduced after ingesting a treatment substance, and the ease to ejaculate regardless of whether the B score was obtained after this effect was noticed (e.g., during measurement two or more C scores for an example) is often referred to herein as the “ease to ejaculate.” Failure to ejaculate occurred in 12 Examples, and of in 3 of those Examples had theanine as a treatment substance; and in Example T37:2 having theanine as a treatment substance the DE score was 1. Of all the Examples, 14 had theanine as a treatment substance, which has various mechanisms of action (see Table 45A). Failure to orgasm occurred in 4 Examples, and spermidine (a carbonic anhydrase inhibitor with other mechanisms of action, see Table 46B) or St. John's Wort (a GABAA-R NAM, with other mechanisms of action, see Table 18A) were treatment substances in 2 of those Examples. Spermidine also had a DE score of 1 in Example T41:84. The time (which was sometimes approximated) and type of a change that may be an additional sexual effect after ingesting a treatment substance, as well as some sexual effects previously described, are shown at Table 44.

TABLE 44
Additional Sexual Effects of Treatment Substances

| | Time of | | Treatment Substance |
|---|---|---|---|
| | Sexual | | (Notable Treatment |
| | Effect | | Substance Associated |
| Example | (Minutes) | Sexual Effect | with Sexual Effect) |
| T36:6 | 60 | C score different | MagT, AKG |
| T36:33 | 60 | Failure to ejaculate | NAC.P, Gly |
| T36:37 | 60 | Failure to ejaculate; C score | NAC.SR, Theanine |
| | | different | |
| T36:39 | 60 | Failure to ejaculate | Theanine |
| T36:40 | 60-318 | At 60 minutes: C score different; at | NGABA, Theanine |
| | | 318 minutes: urination sensitivity | |
| T36:72 | 60 | Failure to ejaculate | NGABA, Tyr |
| T36:94 | 60 | C score different but had more | NGABA, Propolis Extract |
| | | sensitivity | |
| T36:104 | 60 | C score different | Val, Boswellia |
| T36:107 | 65 | Urination sensitivity | Val, Thr |
| T36:110 | 60 | C score different | D-Ser |
| T36:113 | 60 | C score had more sensitivity | (Galantamine HBr) |
| T36:121 | 160 | Urination sensitivity | Val, Leu |
| T36:124 | 80 | Urination sensitivity | NGABA, Alpha-Lipoic Acid |
| T36:129 | 60 | Failure to ejaculate; C score | NGABA, Huperzine A |
| | | different | |
| T36:132 | 60 | C score different | NGABA, DimethylGly |
| T36:139 | 70 | Urination sensitivity | Val, Lemon Balm |
| T36:144 | 60 | Failure to ejaculate | (95% Honokiol/Magnolol) |
| T36:149 | 100 | Urination sensitivity | Val, Pregnenolone |
| T36:154 | 60 | Unable to go from platau phase C | St. John's Wort |
| | | score to orgasm (no A and B scores) | |
| | | and failure to ejaculate | |
| T36:231 | 60 | B score felt different and not as | NGABA, MagT, NAC.P, |
| | | intense | Kava, Saffron Extract, |
| | | | Huperzine A |
| T36:234 | 90 | Urination sensitivity strong | Val, MagT, NAC.P, Saffron |
| | | | Extract, Huperzine A, Kava, |
| | | | Mucuna Pruriens |
| T36:260 | 60 | C score different and not at intense | NGABA, MagT, NAC.SR, |
| | | | Gly, R-Alpha-Lipoic Acid |
| T36:263 | 60 | C score different | NGABA, Alpha-Lipoic Acid |
| T36:264 | 60 | C score different; small semen | NGABA, Yohimbe Bark |
| | | volume | |
| T36:265 | 60 | Erection difficult to achieve | NGABA, Acetyl-Carnitine |
| | | | HCl |
| T36:268 | 60-65 | Urination sensitivity strong | Val, MagT, NAC.P, |
| | | | (Galantamine HBr) |
| T36:271 | 65 | Urination sensitivity | Val, MagT, NAC.P, D-Ser, |
| | | | DimethylGly, (Galantamine |
| | | | HBr) |
| T36:273 | 60 | C score different | (95% Honokiol/Magnolol) |
| T36:274 | 60 | Unable to go from platau phase C | Homotaurine, NAC.P |
| | | score to orgasm (no A and B scores) | |
| | | and failure to ejaculate | |
| T36:276 | 60-70 | At 60 minutes: no C Score skin | Val, Calcium Pyruvate |
| | | sensitivity during A and B score | |
| | | measurements; at 70 minutes: | |
| | | urination sensitivity | |
| T36:279 | 60 | Erection easily achieved | Dextromethorphan |
| T36:280 | 59 | Some C score when penis flaccid | Pramiracetam |
| T36:281 | 59 | Some C score when penis flaccid | Phenylpiracetam |
| T37:1 | 60 | Failure to ejaculate | MagT, Theanine, Inositol |
| T37:31 | 60 | Unable to go from platau phase C | NGABA, NAC.P, |
| | | score to orgasm (no A and B scores) | Ketoisocaproate |
| | | and failure to ejaculate | |
| T37:39 | 65 | Urination sensitivity | NGABA, MagT, NAC.P, |
| | | | (Galantamine HBr) |
| T37:44 | 198 | Urination sensitivity | NGABA, NAC.P, |
| | | | Alpha-GlyceroPhosphoCholine, |
| | | | DimethylGly, (Galantamine |
| | | | HBr) |
| T37:46 | 41 | Ease to ejaculate strongly promoted | Val, MagT, NAC.P, |
| | | | DimethylGly, (Galantamine |
| | | | HBr), Huperzine A, D-Ser, |
| | | | 50% Trans-Resveratrol |
| T37:51 | 60 | A and B scores strong, but also | Val, MagT, NAC.P, 95% |
| | | different with less sensation | Baicalin |
| T37:58 | 60 | A and B scores more localized to | Val, NAC.P, Norvaline |
| | | ureathra | |
| T37:81 | 47 | Ease to ejaculate strongly promoted | Val, NAC.P, Pramiracetam |
| T38:3 | 70 | Urination sensitivity | NGABA, Homotaurine |
| T39:14A | 10 | Ease to ejaculate promoted | Val, NAC.P, 95% PiperineC |
| T40:1 | 35-80 | At 35-70 minutes: erection easily | Val, MagT, NAC.SR |
| | | achieved; at 80 minutes: urination | |
| | | sensitivity | |
| T40:6 | 70-80 | Failure to ejaculate | Val, MagT, NAC.SR, |
| | | | NGABA, NAC.P |
| T41:2 | 40-50 | At 40 minutes: ease to ejaculate | Incarvillea sinensis |
| | | strongly promoted; at 50 minutes: | |
| | | ease to ejaculate promoted | |
| T41:3 | 40-50 | At 40 minutes: erection slightly | Gotu Kola |
| | | difficult to achieve; at 50 minutes: | |
| | | ease to ejaculation slightly promoted | |
| T41:14 | 50 | Skin on penis and hands felt warm | Mucin/Sialac Acid Preparation |
| T41:24 | 30-60 | Erection slightly difficult to achieve | Incarvillea sinensis |
| T41:26 | 40 | Erection slightly difficult to achieve | Fisetin |
| T41:30 | 30-60 | Erection slightly difficult to achieve | 60% Caprylic Acid |
| T41:34 | 40-60 | Erection slightly difficult to achieve | Licorice |
| T41:35 | 30-40 | Erection slightly difficult to achieve | 50% SAMe |
| T41:47 | 60 | Urethra had 6.5 A and B scores | Palmitoylethanolamide |
| T41:60 | 30-60 | Erection easily achieved | Valerian Root Extract, |
| | | | Valerian Root |
| T41:83 | 60 | Unable to go from platau phase C | (1% Spermidine) |
| | | score to orgasm (no A and B scores) | |
| | | and failure to ejaculate | |
| T41:135 | 30-60 | Ease to ejaculate slightly promoted | Val, NAC.P, Turkey Rhubarb |
| T41:136 | 30-60 | Erection slightly difficult to achieve | Val, NAC.P, Alpha-Glycosyl |
| | | | Isoquercitrin |
| T41:137 | 30-60 | At 30 minutes: ease to ejaculate | Val, NAC.P, |
| | | strongly promoted; at 60 minutes: | D-Beta-Hydroxybutyrate |
| | | ease to ejaculate extremely | Ester, Potassium |
| | | promoted | |
| T41:140 | 40 | Ease to ejaculate promoted | (Galantamine HBr) |
| T41:145 | 50 | Ease to ejaculate promoted | Val, NAC.P, Noopept |

It is contemplated that as the highest score for the C score in Table 35 for the no treatment substance ingested measurement was 0.8, and the highest scores for the A or B scores was 8.8, a C score of 1 or more, and either a single score for the A or B scores of 9 or more and/or a plurality of scores at 8 or more for treatment substances) is indicative that the treatment substance(s) is having a positive effect on SRSS rather than the measurement being a false positive score that would have occurred without the treatment substance(s) at the time of measurement. For the C score after ingesting a treatment substance: a score of 2.0 or better is preferred, a score of 3.0 or better more preferred, a score of 4.0 or better even more preferred, and a score of 5.0 or better particularly preferred. For the A and/or B scores after ingesting a treatment substance: a score of 8.0 or better is preferred, a score of 9.0 or better more preferred, and a score of 10.0 or better particularly preferred. Table 45A and Table 45B show Examples of treatment substance(s) that achieved a preferred A/B score of at least 8.0 in combination with a preferred C score of at least 2.0, and/or a more preferred A/B score of at least 9.0 regardless of the C score, when the C, A and B scores were measured at about the same time (e.g., typically at about 60 minutes). Table 45A describes mechanisms of action for the treatment substances. Many treatment substances have multiple mechanisms of action (e.g., a Positive Glutamatergic and a Positive Cholinergic), and sometimes opposing mechanisms of action (e.g., a Positive Glutamatergic and a Positive GABAergic). For example, a treatment substance, such as an enzyme cofactor/enzymatic reaction cosubstrate, may be a promoter of metabolic synthesis of a neurotransmitter in a series of enzymatic reactions and a promoter of the degradation of the same neurotransmitter in another series of enzymatic reactions. For example, a treatment substance at a low dose may activate a receptor to produce an excitatory neurotransmission signal in a neuron but a relatively larger dose may activate a different receptor to produce an inhibitory neurotransmission signal in the same or different neuron. For example, in instances where a treatment substance has multiple mechanisms of action, one (or more) mechanism of action may have a greater effect on treating a sexual dysfunction than another mechanism of action possessed by the treatment substance. In another example, a treatment substance that has the same mechanism of action may have a stronger effect on treating a sexual dysfunction than another treatment substance having the same/similar mechanism of action (e.g., a full agonist having superior effect relative to a partial agonist for a receptor involved in TSF).

TABLE 45A
Treatment Substances With a Preferred C, A or B Score

| Treatment | |
|---|---|
| Substance | Classification [Mechanism of Action] |
| Homotaurine | Positive/Negative GABAergic [GABAA-R Partial Agonist; GABAB-R |
| | Partial Antagonist/Partial Agonist] |
| NGABA | Positive GABAergic [Prodrug that Crosses the BBB for GABA and Niacin |
| | (GABAA-R PAM); Possibly GABAA-R/GABAARho-R/GABAB-R |
| | Agonist] |
| Val | Positive GABAergic [GABA Precursor]; Positive Glutamatergic [Glu |
| | Precursor]; Possible Positive Glutamatergic/GABAergic [For mice, Promotes |
| | new Glu synthesis and Release into the Extracellular Space]; |
| | Negative/Positive Glutamatergic [Glutamate Dehydrogenase1 PAM] |
| Ile | Positive GABAergic [GABA Precursor]; Positive Glutamatergic [Glu |
| | Precursor]; Negative/Positive Glutamatergic [Glutamate Dehydrogenase1 |
| | PAM] |
| Leu | Positive GABAergic [GABA precursor]; Positive Glutamatergic [Glu |
| | precursor]; Negative/Positive Glutamatergic [Glutamate Dehydrogenase1 |
| | PAM] |
| GABA | Positive GABAergic [GABAA-R/GABAARho-R/GABAB-R Agonist]; |
| | Little Movement from Blood Across the BBB to Brain |
| Taurine | Transporter Activator [ASCT2/EAAT1/EAAT2/EAAT3/EAAT4 Activator]; |
| | Positive GABAergic [GAT2/GAT3 GABA Reuptake Inhibitor; |
| | GABAA-R/GABAARho-R Agonist; Possible GABAB-R Agonist]; Positive |
| | Glycinergic [Gly-R Agonist] |
| Ashwagandha | Positive GABAergic [Active: Withanolide (e.g., Withaferin A) (GABAA-R |
| | Agonist/PAM); Active: Withanolide (e.g., Withaferin A)/Triethylene Glycol |
| | (GABAARho-R Agonist/PAM)] |
| Tocopheryl | Positive Glutamatergic/Positive GABAergic [Active: Succinate (Glutaminase |
| Succinate | Activator; May Reduce GABA Degradation through the Succinic |
| | Semialdehyde Dehydrogenase and GABA Transaminase pathway); Active: |
| | Tocopheryl (Antioxidant)] |
| MagT | Positive GABAergic [Active: Magnesium/Mg2+ (GABAA-R |
| | Benzodiazepine Site PAM)]; Negative Glutamatergic [Active: |
| | Magnesium/Mg2+ (NMDA-R Channel Blocker; NMDA-R with GluN2 |
| | Subunit NAM/Antagonist)], Positive D-Serotoninergic [Active: |
| | Magnesium/Mg2+ (Serine Racemase Activator)]; Positive Cannabinoidergic |
| | [Active: Magnesium/Mg2+ (N-Acylphosphatidylethanolamine-Hydrolyzing |
| | Phospholipase D Activator)]; Preparation of Magnesium that More Readily |
| | Crosses BBB than Other Magnesium Salts |
| Valerian | Positive GABAergic [Active: Valerenol, Valerenic Acid, Valeric Acid, |
| Preparation | Isovaleric Acid (GABAA-R Benzodiazepine Site PAM); Active: Valerenic |
| | Acid (GABAA-R with Beta3 Subunit Agonist/Glutamate Decarboxylase |
| | Activator)]; Positive Serotoninergic [Active: Valerenic Acid (5HT5A-R |
| | Partial Agonist)]; Other Active: Isovaleramide, Valepotriate, |
| | Hydroxyvalerenic Acid, Acetoxyvalerenic Acid |
| Boswellia | Positive GABAergic [Active: Dehydroabietic Acid, Incensole, Incensole |
| | Acetate (GABAA-R Non-Benzodiazepine Site PAM)] |
| Kava | Positive GABAergic [Active: Kavalactone (e.g., Desmethoxyyangonin, |
| Preparation | Kavain, Dihydrokavain, Methysticin, Yangonin) (GABAA-R Not |
| | Benzodiazepine Site PAM)]; Positive Cannabinoidergic [Active: Yangonin |
| | (CB1-R Agonist)]; Negative Adrenalinergic/Noradrenalinergic [Active: |
| | Kavain, Dihydrokavain (Reduces Beta-Adrenergic-R Amount)], Positive |
| | Monoaminergic [Active: Kavain, Dihydrokavain (Inhibits MAO B)] |
| Saffron | Positive GABAergic [Active: Safranal, Crocetin, Dimethylcrocetin |
| Preparation | (GABAA-R Agonist/Benzodiazepine Site PAM)]; Positive Cholinergic |
| | [Active: Safranal, Crocetin, Dimethylcrocetin (Acetylcholinesterase |
| | Inhibitor)]; Negative Glutamatergic [Active: |
| | Transcrocetin/Crocetin/Crocin/Safranal (NMDA-R Antagonist)]; Positive |
| | Monoamergic [Active: Crocin, Safranal (Dopamine/Noradrenaline/Serotonin |
| | Reuptake Inhibitor)]; Crocus sativus Preparation [Components comprise |
| | Safranal, Picrocrocin, and Crocin which are hydrolyzed in the |
| | Gastrointestinal tract into Crocetin/Trans-Crocetin] |
| Theanine | Positive/Negative Glutamatergic [NMDA-R Inhibitor/Glu Site Agonist; |
| | AMPA-R/Kainate-R Antagonist; mGlu1-R Agonist; Inhibits Glu and Gln |
| | Transporter Reuptake]; Cerebral Vasodialator that Counteracts Caffeine |
| | Cerebral Vasoconstriction |
| Baicalin | Positive/Negative GABAergic [Active: Baicalin, Baicalein, Wogonin |
| Preparation | (GABAA-R Benzodiazepine Site PAM); Active: Oroxylin A (GABAA-R |
| | NAM)]; Positive/Negative Glutamatergic [Active: Baicalin (Increases |
| | AMPA-R Amount/Decreases NMDA-R Amount)]; Positive Monoaminergic |
| | [Active: Baicalin (Monoamine Oxidase A/B Inhibitor); Active: Oroxylin A |
| | (DAT Inhibitor)]; Transporter Inhibitor [Active: Baicalein, Baicalin (Inhibits |
| | Alpha-Glucosidase)]; Positive Neuropeptidergic [Prolyl Oligopeptidase |
| | Inhibitor (Prevents Degradation of Some Neuropeptides)]; BDNFic [Active: |
| | Baicalein (Increases BDNF Amount)] |
| Lemon Balm | Positive GABAergic [Active: Citranellal, Geraniol, Oleanolic Acid, |
| Preparation | Rosmarinic Acid, Ursolic Acid, Pentacyclic Ursolic Acid (GABA |
| | Transaminase Inhibitor); Active: Rosmarinic Acid (Decreases GABA |
| | Transaminase Amount)]; Positive Cholinergic [Active: Gallic Acid, |
| | Rosmarinic Acid, Citral (Acetylcholinesterase Inhibitor)]; Positive |
| | Monoaminergic [Active: Citranellal, Geraniol (Monoamine Oxidase A |
| | Inhibitor)]; Transporter Inhibitor [Active: Harmine (See Harmala Herein)]; |
| | Other Active: Geranial, 1,8-Cineole, Neral, Citronellal, Geranyl-Acetate, |
| | Beta-Caryophyllene, Beta-Caryophyllene-Oxide. |
| Trans-Resveratrol | Positive/Negative GABAergic [Active: Trans-Resveratrol (Promotes KCC2 |
| Preparation | gene expression; Inhibits Carbonic Anhydrase I/II/VII)]; Positive Cholinergic |
| | [Active: Trans-Resveratrol (Acetylcholinesterase Inhibitor)]; Transporter |
| | Inhibitor [Active: Trans-Resveratrol (MRP4 Inhibitor)]; Positive BDNFic |
| | [Active: Trans-Resveratrol (Increases BDNF Amount)] |
| Chrysin | Positive/Negative GABAergic [Active: Chrysin (GABAA-R Benzodizepine |
| | Site PAM; GABAA-R/GABAB-R Agonist/Antagonist; Carbonic Anhydrase |
| | XII Inhibitor)]; Transporter Inhibitor [Active: Chrysin (P-GP/BCRP/MCT1 |
| | Inhibitor)]; Positive BDNFic Activator [Active: Chrysin (Increases BDNF |
| | Amount)]; Other Active: Harmine (See Harmala Herein) |
| Piperine | Possible Positive GABAergic [Active: Piperine (TRPV1-R Agonist, and |
| Preparation | TRPV1-R Activation Promotes KCC2 Protein Expression)] |
| Panax ginseng | Negative Glutamatergic [Active: Ginsenoside (e.g., |
| Prepartion | Rb1/Rb2/Rc/Re/Rf/Rg1/Rg2/Rg3/Rh1/Rh2) (Inhibits NMDA-R's Increase in |
| | Ca2+ in neurons)]; Positive Cholinergic [Active: Ginsenoside (e.g., |
| | Ginsenoside Rg1/Rb1) (Acetylcholinesterase Inhibitor/Choline |
| | Acetyltransferase Activator)]; Positive Glycinergic [Active: Ginsenoside |
| | (e.g., Ginsenoside-Rf) (Gly-R PAM)]; Negative |
| | Dopaminergic/Noradrenalinergic/Adrenalinergic [Active: Saponins (Reduces |
| | Tyrosine Hydroxylase/Dopamine-Beta-Hydroxylase Amount)]; Transporter |
| | Activator [P-GP Activated at 150 mg/Kg]; Negative Nitroergic [Active: |
| | Ginsenoside Rb1/Rg1 (Nitric Oxide Synthase Inhibitor)]; Positive BDNFic |
| | [Active: Saponin (Increases BDNF Amount)] |
| Huperzine A | Positive Cholinergic [Acetylcholinesterase Inhibitor]; Negative |
| | Glutamatergic [NMDA-R Polyamine Site Antagonist] |
| Galantamine | Positive Cholinergic [Nicotinic ACh-R PAM; Acetylcholinesterase / |
| HBr | Butyrylcholinesterase Inhibitor]; Transporter Inhibitor [OCTN2] |
| Centrophenoxine | Positive Cholinergic/Cholineic [Dimethylethanolamine Prodrug; |
| | Dimethylethanolamine Increases Acetylcholine Amount] |
| Alpha-GlyceroPhosphoCholine | Positive Cholinergic [Acetylcholine Precuror] |
| NAC | Positive Cysteinic/GSHic [NAC a Cys Prodrug that Readily Crosses the |
| | BBB, Cys a Cystine (“Cys2”)/GSH Precursor]; Negative Glutamatergic [Cys |
| | Possibly Promotes Glu/Gln Movement from Brain to Blood]; |
| | Negative/Positive Glutamatergic [Cys a GSH/Cys2 Precursor; GSH degraded |
| | into Extrasynaptic Gly/Glu and/or Cys2 Precursor for Xc- Activation for |
| | Extrasynaptic Glu Release, and Extrasynaptic Glu Activates Extrasynaptic |
| | NMDA-R/mGlu(2/3)-R/mGlu5-R; and Activated Extrasynaptic Glu |
| | Receptors (e.g., mGlu2/3) may Reduce Synaptic Release of Glu; and |
| | Activated Extrasynaptic NMDA-R may Increase/Decrease Neurotransmitter |
| | Release; Activated Extrasynaptic, Postsynaptic mGlu5-R Promotes |
| | Glutamatergic Neurotransmission]; Negative D-Serinergic [Cys Serine a |
| | Racemase Inhibitor for Reduced D-Ser Production and NMDA-R (e.g., |
| | Synaptic NMDA-R) Activation]; Positive Glutamatergic/GABAergic [Cys |
| | Increases Extrasynaptic Glu by Xc- that is Moved by EAAT2/3 into |
| | Presynaptic Neuron for Glu/GABA Production and Synaptic Release; and |
| | newly created neurotransmitters (e.g., GABA) may be moved into synaptic |
| | vesicles preferentially over reuptaken neurotransmitters (e.g., Glu) for |
| | synaptic neurotransmission] |
| Cys | Positive Cysteinic [Crosses BBB Less Readily than NAC, See NAC Above |
| | for Mechanisms of Action] |
| DimethylGly | Positive/Negative Glutamatergic [NMDA-R Gly Site (Partial Agonist)] |
| D-Ser | Positive D-Serinergic/Glutamatergic [Coagonist for NMDA-R (e.g., Synaptic |
| | NMDA-R having the GluN2A Subunit)]; Positive Glycinergic [Gly-R |
| | Agonist] |
| 5HTP | Positive Serotoninergic/Melatoninergic [Precursor to Serotonin/Melatonin |
| | that Readily Crosses BBB]; Negative |
| | Dopaminergic/Noradrenalinergic/Adrenalinergic/Catecholaminergic |
| | [Aromatic L-Amino Acid Decarboxylase Competitive Substrate] |
| Calcium | Calcium/Ca2+: Positive GABAergic [KCC2 Activator (by Ca2+ Dependent |
| Pyruvate | PKC Phosphorylation of KCC2 Cell Surface Amount to Promote |
| | GABAergic Signaling)]; Negative Glutamatergic [Ca2+ Inactivates |
| | AMPA-R (by Movement of AMPA-R from Cell Surface)]; Positive |
| | D-Serinergic [Ca2+ Serine Racemase Activator]; Positive Nitroergic [Ca2+ |
| | activates Nitric Oxide Synthase]; Positive Cannabinoidergic [Ca2+ |
| | Activates: N-Acylphosphatidylethanolamine-Hydrolyzing Phospholipase D |
| | Synthesis of Anandamide and Diacylglycerol Lipase-Alpha Synthesis 2-AG]; |
| | Positive Glutamatergic/Cholinergic/Cannabinoidergic [Ca2+ |
| | NMDA-R/AMPA-R/Kainate-R/Nicotinic ACh-R/TRPV1-R |
| | Neurotransmission Ion]; Neurotransmission Promoter [Intracellular Ca2+ |
| | promotes neurotransmitter release by Glial Cell (e.g., Astrocytes)/Neuron by |
| | Vesicle/Non-Vesicle Mechanisms]; and Pyruvate: Negative Glutamatergic |
| | [Pyruvate Inhibits VGLUT2, Blood Pyruvate Promotes Glu Movement from |
| | Brain as a Glutamate Pyruvate Transaminase Substrate] |
| Yohimbine | Positive/Negative Dopaminergic [D1A-R Partial Agonist; D2-R/D3-R/D4-R |
| Preparation | Antagonist]; Negative Adrenalinergic/Noradrenalinergic |
| | [Alpha1-R/Alpha2-R Antagonist]; Negative/Positive Serotoninergic |
| | [5HT1B-R/5HT1D-R/5HT2A-R/5HT2B-R Antagonist; 5HT1A-R Agonist] |
| Collagen | Negative D-Serinergic [Active: Hydroxyproline (Serine Racemase |
| Peptides | Inhibitor)], Positive/Negative Neurotransmitters [Various amino acid |
| | precursors for neurotransmitters]; Transporter Inhibitor [Active: |
| | Hydroxyproline (ASCT1/ASC1 Inhibitor)] |
| Cat's Claw | Positive/Negative Glutamatergic {Active: Rhynchophylline/ |
| Preparation | Isorhynchophylline [NMDA-R Inhibitor (Pore Blocker/Dizocilpine Site |
| | Antagonist)]; Active: Uncarine E [NMDA-R Agonist/PAM]}; |
| | Positive/Negative Cholinergic [Active: Uncarine E, Rhynchophylline |
| | (Nicotinic ACh-R Agonist/PAM); Active: Rhynchophylline (Nicotinic |
| | ACh-R with Alpha3/Beta4 Subunit Agonist/PAM); Active: |
| | Rhynchophylline/Isorhynchophylline (Muscarinic ACh-R Antagonist/NAM); |
| | Active: Oxindole Alkaloids such as Uncarine E, Uncarine C, Mitraphylline |
| | (Muscarinic ACh-R Agonist/PAM)]; Negative Serotoninergic [Active: |
| | Rhynchophylline/Isorhynchophylline (5HT2-R Antagonist/NAM)]; Other |
| | Active: Catechin, Epicatechin |
| Norvaline | Positive Nitroergic [Arginase Inhibitor, so Arg metabolized into Nitric |
| | Oxide]; Negative Polyaminergic [Arg Not converted into Ornithine by |
| | Arginase, Ornithine a Putrascine Precursor]; Negative GABAergic [Reduced |
| | Putrascine Amount to be Metabolized into GABA]; Chemical Structure |
| | Similar to Val |
| Gly | Positive Glycinergic [Gly-R Agonist], Positive/Negative Glutamatergic |
| | [NMDA-R Gly Site Agonist; Positive GSHic (See NAC Herein)], |
| | Positive/Negative D-Serinergic [Serine Racemase Inhibitor; Ser/Gly |
| | Hydroxymethyltransferase Substrate for Ser Production] |
| Pramiracetam | Positive Glutamatergic [AMPA-R PAM]; Positive Glycinergic/Positive |
| | D-Serinergic [Astrocyte AMPA-R Activation Promotes Gly, D-Ser Release]; |
| | Possible Positive Cholinergic [May Increase Cholinergic Signalling]; Prolyl |
| | Endopeptidase Inhibitor |
| Inosine | Positive Adenosinergic [A3-R Agonist; Adenosine Precursor by Adenosine |
| | Deaminase] |
| Caffeine | Negative Adenosinergic [A1-R/A2A-R/A2B-R/A3-R Antagonist]; Negative |
| | Glycinergic [Gly-R Antagonist]; Positive Cholinergic [Acetylcholinesterase |
| | Inhibitor]; Transporter Inhibitor [EAAT3 (Reduces Transporter Amount)] |
| Theobromine | Negative Adenosinergic [A1-R/A2A-R/A2B-R/A3-R Antagonist]; |
| | Phosphodiesterase 4 Inhibitor |
| Cloves | Positive GABAergic [Active: Thymol, Eugenol (GABAA-R PAM)]; Positive |
| Preparation | Cannabinoidergic [Active: Beta-Caryophyllene (CB2-R Agonist)]; Other |
| | Active: Eugenyl Acetate; Methylsalicylate, Methyleugenol, Benzaldehyde, |
| | Methylamyl Ketone, Alpha-Ylangene, Clovinol |
| Ornithine | Positive Glutamatergic/GABAergic [Ornithine Aminotransferase Substrate to |
| | create Glu; Glu Precursor to GABA; Precursor to GABA in Polyamine |
| | Metabolism]; Positive Polyaminergic [Ornithine Decarboxylase Substrate to |
| | create Putrescine]; Positive GHBergic [Precursor to GHB in Polyamine |
| | Metabolism] |
| Icariin | Positive Vasodilator [Active: Icariin (Phosphodiesterase Inhibitor 5)]; |
| Preparation | Positive Nitroergic/Nitric Oxideic [Active: Icariin (Increases Nitric Oxide |
| | Amount and Vasodilation)]; Active: Icariin (Increases Corticosterone |
| | Receptor and Glucocorticoid Receptor Amount) |
| Gln/Glu | Positive Glutamatergic [Gln is Glu Precursor; Glu Agonist for |
| | NMDA-R/AMPA-R/mGlu-R], Positive GABAergic/GSHic [Glu is GABA |
| | and GSH Precursor; GABA Agonist for GABA-R]; Little Movement from |
| | Blood Across BBB into Brain |
| Alpha-Lipoic | Negative Glutamatergic [Increases Glutamate Cysteine Ligase mRNA |
| Acid Preparation | Expression to Increase Intercellular GSH; Protects GSH from Oxidation]; |
| | Negative Glutamatergic/GABAergic [Branched-Chain Alpha-Keto Acid |
| | Dehydrogenase Complex Cofactor]; Possible Positive Cholinergic [Possible |
| | Choline Acetyltransferase Cofactor]; AKG Degradation Promoter [AKG |
| | Dehydrogenase Complex Cofactor; See AKG at Table 45A]; R-Fraction |
| | Alpha-Lipoic Acid has Greater Bioavailability |
| Tyr/AcetylTyr | Negative Glutamatergic/GABAergic [Competitive Inhibitor BCAA |
| | movement by LAT1]; Positive Dopaminergic [D1-R/D5-R/D2-R/D3-R/D4-R |
| | Agonist; Precursor to Dopamine]; Positive/Negative |
| | Adrenalinergic/Noradrenalinergic [Alpha1-R/Alpha2-R/Beta1-R Agonist; |
| | Precursor to Adrenaline/Noradrenaline; Aromatic L-Amino Acid |
| | Decarboxylase Competitive Substrate]; Positive Tyraminergic [Precursor to |
| | Tyramine]; AcetylTyr is Try Prodrug |
| Oleamide | Positive Cannabinoidergic [CB1-R Agonist; Competitive Substrate for Fatty |
| | Acid Amide Hydrolase that Degrades Anandamide/2-AG that are |
| | CB1-R/CB2-R Agonists] |
| Acetyl-Carnitine | Transporter Inhibitor {Active: Acetyl-Carnitine [ATB(0, +) Inhibitor]}; |
| Preparation | Possible Negative D-Serinergic {Active: Acetyl-Carnitine [Inhibits |
| | ATB(0, +)]}; Possible Positive GABAergic/Ketogenesis Promoter [Active: |
| | Acetyl-Carnitine (May increase fat metabolism/reduce BCAA degradation |
| | for ATP production)]; Positive/Negative Glutamatergic [Active: |
| | Acetyl-Carnitine (Modulates NR1 subunit amount for NMDA-R; Increases |
| | mGlut2-R in the cerebral cortex/spinal cord upon days of ingestion)]; Active: |
| | Acetyl-Carnitine Prodrug for Carnitine that is More BBB permeable than |
| | Carnitine |
| Trp | Positive Serotoninergic/Melatoninergic/Tryptaminergic [Precursor to |
| | Serotonin/Melatonin/Tryptamine]; Negative Dopaminergic/ |
| | Noradrenalinergic/Adrenalinergic/Catecholaminergic [Aromatic L-Amino |
| | Acid Decarboxylase Competitive Substrate]; Negative Glutamatergic/ |
| | GABAergic [Competitive Inhibitor BCAA movement by LAT1] |
| AKG | Positive Glutamatergic/GABAergic [Promotes Production of GABA |
| | Precursor Glu by Glutamate-Pyruvate Transaminase/Alanine |
| | Aminotransferase/Glutamate Oxaloacetate Transaminase/Glutamate |
| | Dehydrogenase/Ornithine Aminotransferase/Liver Tyrosine |
| | Aminotransferase/BCATase]; Negative GABAergic [Promotes GABA |
| | Degradation by GABA Transaminase]; Transporter Inhibitor [OAT3 |
| | Inhibitor] |
| D-Asp | Positive Glutamatergic [NMDA-R/mGlu5-R Glu Site Agonist (See NMDA |
| | Table 46B)]; Transporter Activator [EAAT1 Amount Increased]; Possible |
| | Negative D-Serinergic [D-Ser Competitive Inhibitor for ATB(0, +) |
| | Transporter]; Positive Neurosteroidic/Steroidic [May Increase Testosterone |
| | After Days of Ingestion] |
| Sarcosine | Positive Glutamatergic [NMDA-R Gly Site Agonist]; Positive/Negative |
| | Glycinergic [Gly-R Agonist; GLYT1/SIT1 Release/Reuptake Inhibitor]; |
| | Transporter Inhibitor [PROT Inhibitor] |
| Betaine | Positive/Negative GABAergic [GAT2/BGT1 Release/Reuptake Inhibitor; |
| | Inhibits BGT1 Movement of GABA Out of the Brain]; Positive |
| | Glutamatergic [NMDA-R Gly Site (Partial Agonist)]; Positive Glycinergic |
| | [SIT1 Reuptake Inhibitor]; Transporter Inhibitor [OCTN2 Inhibitor] |
| Pyroglutamate | Possible Negative Glutamatergic [Pyroglutamate inside a cell promotes |
| | ASCT2/EAATs movement of Glu/Gln from Brain]; Transporter Activator |
| | [ASCT2/EAAT1/EAAT2/EAAT3 Activator; Pyroglutamate inside a cell |
| | promotes extracellular amino acid (e.g., Glu) movement into the cell]; |
| | Positive Glutamatergic/GABAergic [Pyroglutamate Glu Precursor, Glu |
| | Precursor to GABA] |
| Mucuna | Positive Dopaminergic/Adrenalinergic/Noradrenalinergic [Active: DOPA |
| Pruriens | (Dopamine Precursor; Dopamine Metabolized into Adrenaline / |
| | Noradrenaline; D1-R/D5-R/D2-R/D3-R/D4-R/Alpha1-R/Alpha2-R / |
| | Beta1-R Agonist)]; Negative Glutamatergic/GABAergic [Competitive |
| | Inhibitor of BCAA movement by LAT1]; Negative Serotoninergic/Trace |
| | Aminergic [Aromatic L-Amino Acid Decarboxylase Competitive Substrate]; |
| | Active: DOPA Readily Crosses the BBB From the Blood into the Brain |
| | Relative to Dopamine |
| Riboflavin/ | Negative/Positive Glutamatergic/GABAergic [Riboflavin/Pyridoxine |
| Pyridoxine | Cofactors in BCAA Metabolism Enzymes, Pyridoxine AAA Decarboxylase |
| | Cofactor] |
| Beta-Ala | Positive/Negative GABAergic [GAT1/2/3 Inhibitor, BGT1 Inhibitor, |
| | GABAA-R/GABAARho-R Agonist; VGAT Inhibitor]; Positive Glycinergic |
| | [GLYT2 Inhibitor, Gly-R Agonist]; Transporter Inhibitor [TAUT Inhibitor] |
| Cinchona | Transporter Inhibitor [Active: Quinidine/Quinine |
| Preparation | (OCTN2/P-GP/OCT1/OCTN1 Inhibitor)]; Positive GABAergic [Active: |
| | Quinidine (GAT2/GAT3/BGT1 Inhibitor)]; Negative Cholinergic [Active: |
| | Quinidine (Muscarinic ACh-R Antagonist)]; Other Active: Quinoline |
| Bee Propolis | Transporter Inhibitor [Active: Pinocembrin (P-GP Inhibitor)]; Antioxidant |
| Preparation | |
| Inositol | Neurotransmission Activator [Incorporated Into Various Brain Biochemicals |
| | such as brain phospholipids and second messenger systems activated by |
| | neurotransmitter receptor activation, and metabolized into components |
| | involved in ions crossing brain cell membranes] |
| Ubiquinone | Antioxidant |
| Noopept | Possible Negative Glutamatergic [Possible mGlu2-R Agonist (Activation of |
| | Extrasynaptic mGlu2-R on a Presynaptic Neuron May Reduce Synaptic Glu |
| | Release)]; Antioxidant |
| Vanilla | Possible Positive GABAergic [Active: Vanillin (TRPV1-R Agonist, and |
| Preparation | TRPV1-R Activation Promotes KCC2 Protein Expression)] |
| Thr | Positive Glycinergic [Gly-R Agonist]; Transporter Inhibitor |
| | [LAT1/ASCT2/ASCT1/ASC1/ATB(0, +) Competitive Inhibitor for |
| | Movement of Other Amino Acids such as BCAAs] |

Table 45B show Examples of treatment substance(s) that achieved a preferred C, A, and/or B scores, with the treatments substance(s) categorized by a contemplated mechanism of action for each treatment substance that may contribute to the preferred score(s). In the examples shown at Table 45B there were about 40 occurrences of NAC.P and 20 occurrences of NAC.SR being ingested for the preferred C scores, and about 82 occurrences of NAC.P and 20 occurrences of NAC.SR being ingested for the preferred A and/or B scores, but both preparation types are referred to as “NAC” in Table 45B. Water was ingested for all examples from Table 36, Table 41 and examples T39:1, T39:5, T39:14 A, T39:15, T39:16, T40:2, and T40:12; coffee, which contains the treatment substance caffeine, was ingested for all examples from Table 37 and examples T39:4, T40:1, T40:3A, T40:3B, T40:4A, T40:4B, T40:5, T40:6, T40:7, T40:8, T40:9, T40:10, T40:11, and T40:13; and wine/beer, which contains the treatment substance alcohol (which has many mechanisms of action), was ingested for all examples from Table T38 and examples T39:2 and T39:11. The legend at the bottom of Table 45B explains the differences in bolded font/parenthesis/brackets/braces as related each Example's scores, with Examples having relatively higher scores being emphasized by bolding and then additional parenthesis/brackets/braces to further associate a contemplated mechanism of action with examples producing more preferred scores.

TABLE 45B
Treatment Substances With a Preferred C, A and/or B Score
Classification of: Treatment Substance\Treatment Substance\\Treatment Substance

| | | |
|---|---|---|
| C Score | A/B Score | Treatment |
| Example* | Example** | Substance |

Positive Cholinergenic

| | | |
|---|---|---|
| T36:193 | T36:193 | Panax ginseng |

Positive GABAergic

| | | |
|---|---|---|
| T38:18, | [T38:18] | Homotaurine |
| T38:5, T37:57 | [T38:5], [T37:57] | |
| T36:157 | [T36:157] | Homotaurine, MagT |
| T38:3 | [T38:3] | Homotaurine, NGABA |
| T36:64 | T36:64 | NGABA, MagT |
| T36:203 | T36:203 | NGABA, MagT, Ashwagandha |
| T36:40 | T36:40 | NGABA, Theanine |
| T36:108 | T36:108 | NGABA, Tcopheryl Succinate |
| T36:81 | T36:81 | NGABA, Val |
| T40:9 | [T40:9] | Val, MagT |
| T36:272 | [T36:272] | Val, GABA |
| T36:207 | T36:207 | Ile, Mag |
| T37:65, T36:204 | T37:65, T36:204 | NGABA, Val, MagT |
| T37:88 | [T37:88] | Val, Leu, Ile, MagT |
| T37:7 | T37:7 | Val, Ile, Leu |
| T37:70 | T37:70 | Val, Ile, Leu, Gln, Mag |

Positive GABAaeric\Other Treatment Substance

| | | |
|---|---|---|
| T36:276 | [T36:276] | Val\Calcium Pyruvate |
| T37:53 | T37:53 | Homotaurine\DimethylGly |
| T37:54 | [T37:54] | Homotaurine\D-Ser |
| T36:264 | [T36:264] | NGABA\Yohimbe Bark |
| T36:142 | [T36:142] | Val\Inosine |
| T38:14 | [T38:14] | NGABA\Collagen Peptides |
| T36:95 | T36:95 | Val\5HTP |
| T36:217 | T36:217 | Val, MagT\5HTP |
| T36:67 | T36:67 | NGABA, MagT\5HTP |
| T37:66 | T37:66 | Val, MagT\Cat's Claw Bark, Cat's Claw Bark Extract |
| T36:66 | T36:66 | NGABA, MagT\Oleamide |

Positive GABAergic\Positive Cysteineic

| | | |
|---|---|---|
| T36:69, T39:16 | T36:69, [T39:16] | NGABA\NAC |
| T38:8, T36:35 | [T38:8], T36:35, | Val\NAC |
| T36:43, T36:44, | [T36:43], T36:44, | |
| {[T37:67]}, | [T37:67], | |
| T40:8 | {[T40:8]} | |
| T37:26 | [T37:26] | Val\NAC, Cys |
| T36:30, T39:11, | T36:30, {[T39:11]}, | Val, MagT\NAC |
| T36:65 | [T36:65] | |
| T36:230 | [T36:230] | NGABA, Saffron Extract\NAC |
| T36:41, T36:38 | T36:41, T36:38 | NGABA, Theanine\NAC |
| T36:31, T36:215, | T36:31, T36:215, | Val, MagT\NAC |
| [T37:17], | [T37:17], | |
| T37:25, T37:68, | T37:25, [T37:68], | |
| T40:1 | {[T40:1]} | |
| T36:227, | T36:227, | Val, Saffron Extract\NAC |
| T36:192 | T36:192 | |
| T41:111 | T41:111 | Val, Valerian Root Extract, Valerian Root\NAC |
| T41:112 | T41:112 | Val, Lemon Balm Extract\NAC |
| T39:14A | [T39:14A] | Val, 95% PiperineC\NAC |
| T41:146 | [T41:146] | Val, Vanilla\NAC |
| T37:27 | [T37:27] | Val, MagT, \NAC, Cys |
| T40:6, T37:23, | T40:6, {[T37:23]}, | NGABA, Val, MagT\NAC |
| T40:7, T36:161, | {[T40:7]}, [T36:161], | |
| T37:24 | [T37:24] | |
| T37:73 | T37:73 | NGABA, MagT, Boswellia\NAC |
| T37:72 | T37:72 | Val, MagT, Boswellia\NAC |
| T36:151 | [T36:151] | NGABA, 95% Baicalin\NAC |
| T37:51 | [T37:51] | Val, MagT 95% Baicalin\NAC |
| T41:138 | [T41:138] | Val, Ornithine\NAC |
| {[(T36:209)]} | T36:209 | Val, MagT, Taurine\NAC |
| T37:33 | T37:33 | Val, MagT, Tocopheryl Succinate\NAC |
| T36:228 | T36:228 | Val, MagT, Saffron Extract\NAC |
| T36:229 | T36:229 | Val, Saffron Extract, Theanine\NAC |
| T39:15 | [T39:15] | Val, MagT, Saffron Extract, 95% PiperineC, Lemon |
| | | Balm\NAC |

Positive GABAergic\Positive Cysteineic\\Other Treatment Substance

| | | |
|---|---|---|
| T37:29 | T37:29 | Val\NAC\\5HTP |
| {[T36:218]} | [T36:218] | Val, MagT\MAC\\5HTP |
| T37:16, T37:13, | T37:16, T37:13, | Val, MagT\NAC\\Oleamide |
| T37:15 | T37:15 | |
| T37:82 | T37:82 | Val\NAC\\Icariin |
| T36:210 | T36:210 | Val, MagT\NAC\\Acetyl-Carnitine HCl |
| T38:13 | T38:13 | Val, MagT\NAC\\Propolis Extract |
| T40:11 | {[(T40:11)]} | Val, MagT\MAC\\D-Asp, Caffeine |
| T41:113 | [T41:113] | Val\NAC\\99% Theobromine |
| T37:19 | T37:19 | Val, Ile, Leu, MagT\NAC\\Riboflavin, Pyridoxine |
| T36:253 | [T36:253] | NGABA, Saffron Extract\NAC\\Collagen Peptides |
| T37:34 | [T37:34] | Val, MagT, Theanine\NAC\\Inositol |
| T37:62 | [T37:62] | Leu, MagT\NAC\\Inosine |
| T37:52 | [T37:52] | NGABA, Val, MagT, 95% Baicalin\NAC\\Inosine |
| T37:36 | [T37:36] | Val, MagT, \NAC\\D-Ser, DimethylGly |
| T37:59 | [T37:59] | Val, MagT\NAC\\DimethylGly, Sarcosine, Betaine |
| T37:81, T37:84, | [T37:81], [T37:84], | Val\NAC\\Pramiracetam |
| T40:13 | {[(T40:13)]} | |
| T37:58, T38:10 | [T37:58], [T38:10] | Val\NAC\\Norvaline |
| T37:60 | [T37:60] | |
| T41:120 | T41:120 | Val\NAC\\Cloves Extract |
| T41:128 | T41:128 | Val, 95% PiperineS\NAC\\Chrysin |
| T41:145 | [T41:145] | Val\NAC\\Noopept |

Positive GABAergic\Positive Cholinergic\\Another Treatment Substance

| | | |
|---|---|---|
| T36:199 | [T36:199] | NGABA \ Huperzine A \\ Calcium Pyruvate |

Positive GABAergic\Positive Cysteineic\\Positive Cholinergic

| | | |
|---|---|---|
| T36:196 | T36:196 | Val\NAC\\Panax ginseng |
| T37:40 | [T37:40] | Val\NAC\\Galantamine HBr |
| T37:39 | [T37:39] | NGABA, MagT\NAC\\Galantamine HBr |
| T37:41, | [T37:41], | Val, MagT\NAC\\Galantamine HBr |
| T36:268, T37:37 | [T36:268], [T37:37] | |
| T37:50 | [T37:50] | Val, MagT\NAC\\Centrophenoxine |
| T37:38 | [T37:38] | Val, MagT\NAC\\Galantamine HBr |
| | | Alpha-GlyceroPhosphoCholine |
| T36:231 | [T36:231] | NGABA, MagT, Saffron Extract, |
| | | Kava\NAC\\Huperzine A |
| T36:232 | [T36:232] | Val, MagT, Saffron Extract, |
| | | Kava\NAC\\Huperzine A |
| T37:85 | [T37:85] | Val, Saffron Extract, Kava, |
| | | Valerian\NAC\\Huperzine A |
| T36:277 | [T36:277] | Val, MagT, Saffron Extract, |
| | | Kava, Lemon Balm\NAC.P\\Huperzine A |

Positive GABAergic\Positive Cysteineic\\Positive Cholinergic\\\Another Treatment Substance

| | | |
|---|---|---|
| T36:241 | [T36:241] | NGABA, MagT, Saffron Extract, |
| | | Kava\NAC\\Huperzine A\\\Pyroglutamate |
| T37:45 | [T37:45] | Val, MagT\NAC\\Galantamine HBr, |
| | | Huperzine A\\\DimethylGly |
| T37:47 | [T37:47] | NGABA, MagT, Leu\NAC\\Galantamine |
| | | HBr, Huperzine A\\\Inosine |
| T37:76 | [T37:76] | HGABA, MagT, Saffron Extract, Lemon |
| | | Balm\NAC\\Huperzine A\\\Caffeine |
| T37:74 | [T37:74] | Val, MagT, Saffron Extract, |
| | | KAVA\NAC\\Huperzine A\\\Caffeine |
| T36:234 | [T36:234] | Val, MagT, Saffron Extract, |
| | | Kava\NAC\\Huperzine |
| | | A\\\Mucuna Pruriens |
| T37:75 | [T37:75] | NGABA, MagT, Saffron Extract, |
| | | Kava\NAC\\Huperzine |
| | | A\\\Beta-Ala |
| T37:77 | [T37:77] | Val, MagT, Saffron Extract, |
| | | Kava\NAC\\Huperzine |
| | | A\\\Beta-Ala |
| T36:271 | {[T36:271]} | Val, MagT\NAC\\Galantamine |
| | | HBr\\\D-Ser, DimethylGly |
| T37:46 | [T37:46] | Val, MagT, 50% Trans- |
| | | Resveratrol\NAC\\Galantamine HBr, |
| | | Huperzine A\\\D-Ser, DimethylGly |
| T37:48 | [T37:48] | Val, MagT\NAC\\Galantamine HBr\\\D-Ser, |
| | | DimethylGly, Alpha-Lipoic Acid, |
| | | Acetyl-Carnitine, Ubiquinone |
| T37:86 | [T37:86] | Val, MagT, Saffron Extract, |
| | | Kava\NAC\\Huperzine A\\\Cinchona |
| T36:237 | [T36:237] | NGABA, MagT, Saffron Extract, |
| | | Kava\NAC\\Huperzine A\\\AcetylTyr |
| T37:49, T38:2 | [T37:49], [T38:2] | Val, MagT\NAC\\Galantamine |
| | | HBr\\\D-Ser, Inosine |
| T37:63 | [T37:63] | Val, MagT, Saffron Extract, |
| | | Kava\NAC\\Huperzine |
| | | A\\\Calcium Pyruvate |

Positive GABAergic\Gly

| | | |
|---|---|---|
| [T38:12] | T38:12 | Val, MagT, Taurine\Gly |
| T36:213 | T36:213 | NGABA, MagT\Gly |

Positive GABAergic\Positive Cysteineic\\Gly

| | | |
|---|---|---|
| T36:258 | T36:258 | NGABA, MagT, Lemon Balm \ NAC \\ Gly |
| T36:11 | T36:11 | NGABA, MagT, Boswellia \ NAC \\ Gly |
| T36:47, T36:51, | T36:47, T36:51, | NGABA\NAC\\Gly |
| T36:52, T36:57, | T36:52, T36:57, | |
| [T36:55] | [T36:55] | |
| [T36:200], | T36:200, | Val\NAC\\Gly |
| T37:61, | [T37:61], | |
| T36:49 | [T36:49] | |
| [T36:202] | T36:202 | NGABA, MagT\NAC\\Gly |
| T36:8, T36:29, | T36:8, T36:29, | |
| T37:5, T39:5, | T37:5, T39:5, | |
| [T40:12], | [T40:12], | |
| T36:48 | [T36:48] | |
| T39:4, T37:71, | T39:4, [T37:71], | Val, MagT\NAC\\Gly |
| T40:10, | {[T40:10]}, | |
| T36:9, | T36:9, | |
| {[T36:220]}, | T36:220, | |
| T38:1, T40:2 | [T38:1], T40:2 | |
| T40:4A, | [T40:4A], | NGABA, Val, MagT\NAC\\Gly |
| T40:5, | [T40:5], | |
| [T40:3A], | [T40:3A], | |
| T40:3B | T40:3B | |
| T36:21 | T36:21 | NGABA, MagT, Glu\NAC\\Gly |
| T37:3 | T37:3 | NGABA, MagT, Gln\NAC\\Gly |
| [T36:1] | T36:1 | MagT\NAC\\Gly |

Positive GABAergic\Positive Cysteineic\\Gly\\\Other Treatment Substance

| | | |
|---|---|---|
| T36:22 | T36:22 | NGABA, MagT\NAC\\Gly\\\Trp |
| T36:261 | T36:261 | NGABA, MagT\NAC\\Gly\\\Yohimbe Bark |
| T36:10 | T36:10 | NGABA, MagT\NAC\\Gly\\\Cat's Claw Bark |
| | | Extract, Cat's Claw Bark |
| T36:25 | T36:25 | NGABA, MagT\NAC\\Gly\\\Thr |
| T36:257 | T36:257 | Val, MagT\NAC\\Gly\\\Yohimbe Bark |
| T36:12 | T36:12 | Val, MagT\NAC\\Gly\\\R-Alpha-Lipoic Acid |
| T36 20 | T36 20 | NGABA, MagT\NAC\\Gly\\\Tyr |
| T37:10 | [T37:10] | Val, MagT\NAC\\Gly\\\Tyr |
| T37:14 | T37:14 | Val, MagT\NAC\\Gly\\\Oleamide |
| T36:17 | T36:17 | NGABA, MagT\NAC\Gly\\\AKG |
| T37:30 | [T37:30] | Val, MagT\NAC\\Gly\\\AKG |
| T37:32 | [T37:32] | Val, MagT\NAC\\Gly\\\Acetyl-Carnitine HCl |
| {[T37:69]} | [T37:69] | NGABA, MagT\NAC\\Gly\\\5HTP |
| T39:2 | T39:2 | Val, MagT\NAC\\Gly\\\5HTP |
| {[T36:212]} | [T36:212] | Val, MagT, Ashwagandha\NAC\\Gly\\\5HTP |
| T36:208 | T36:208 | Ile, MagT, Ashwagandha\NAC\\Gly\\\5HTP |
| T39:1 | T39:1 | NGABA, MagT\NAC\\Gly\\\Propolis Extract |
| T40:4B | [T40:4B] | NGABA, Val, Leu, Ile, Gln, |
| | | MagT\NAC\\Gly III Food |

*Examples where the C score is below 2 in regular, not bold, font; Examples where the C score is 2 or above and below 3 in Bold; Examples where the C score is 3 or above and below 4 [In Bold in Brackets]; Examples where the C score is 4 or above and below 5 {[In Bold in Double Brackets]}; Examples where the C score is 5 or above {[(In Bold in Triple Brackets)]
**Examples with an A and/or B score 8.0 or above but below 9 in regular, not bold, font; Examples with an A or B score of 9 or above but below 10 are in Bold; Examples where both A and B scores are 9 or above and below 10 [In Bold in Brackets]; Examples with an A or B score of 10 or above {[In Bold in Double Brackets]}; Examples where both A and B scores are 10 or above {[(In Bold in Triple Brackets)]}.

An A or B score of 10 or more occurred at Examples T40:8, T40:1, T40:7, T40:13, T40:10, T40:11, T39:11, T37:23, T36:271, with 66.6% occurring in Examples from Table 40B wherein treatment substances were ingested in multiple doses. Of the 15 Examples at Table 40B, 14 Examples had an A or B score of 9 or more and 8 Examples had a C score of 2.0 or more. Ingesting multiple doses of treatment substances also generally prolonged the time the improvements in sexual functions lasted (see Examples T40:1, T40:2, T40:3A, T40:3B, T40:4B, T40:6, T40:7, T40:8, T40:9, T40:10, T40:11, and T40:12) and it is contemplated that ingesting multiple doses of treatment substances further promotes reducing a sexual dysfunction such as SRSS (e.g., improving TSF, increasing ease of ejaculation/orgasm). IT is contemplated that ingesting multiple doses of a treatment substance that is and/or comprises an Active over time promotes: greater bioavailability of the Active (e.g., an Active that is slowly absorbed from the intestines has time to reach maximum blood concentrations); an Active to have more time to be created from a treatment substance (e.g., a prodrug that is metabolized into an Active); and/or an Active from one or more additional doses of a treatment substance to replace the amount of the Active from the earlier dose(s) that was lost to metabolic degradation, excretion, etc. For example, in certain embodiments, one treatment substance may reach maximum effect improving TSF 40 minutes after ingestion, and another treatment substance may reach maximum effect improving TSF 60 minutes after ingestion; and ingesting the different treatment substances together (e.g., both in one capsule) at 60 minutes and together again at 40 minutes before measurements are made of TSF (e.g., during sexual stimulation with a female partner) is contemplated as being preferred for ease of use rather than ingesting one treatment substance alone at 60 minutes (e.g., swallow a capsule from one bottle) and then ingesting another treatment substance at alone 40 minutes (e.g., swallow a tablet from another bottle) before measurements are made. In another example, in other embodiments, a treatment substance that improves TSF may be ingested one or more times and TSF measurements made (e.g., during sexual stimulation with a female partner) before ingesting a dose of a treatment substance that improves ease of ejaculation/orgasm.

An A/B score of 9 or above are contemplated to be sufficient to increase the ability to engage in natural reproduction, particularly when combined with a treatment substance that promotes ease of ejaculation/orgasmic response. A treatment substance that promotes ease of ejaculation/orgasm (e.g., a DE score of above 2.0 such as 3.0), is contemplated to be sufficient to to increase the ability to engage in natural reproduction and is preferred for this function, regardless of the A/B/C and other sexual function score(s).

Shown at Table 45C are examples of a treatment substance that achieved a DE preferred score for promoting ease of ejaculation/orgasm. In the examples shown at Table 45C there were 17 occurrences of NAC.P and 3 occurrences of NAC.SR, but both preparation types are referred to as “NAC” in Table 45C. Water was ingested for all examples from Table 36, Table 39 and Table 41. Coffee or wine/beer was ingested for all examples from Table 37 and Table 38, respectively. The legend at the bottom of Table 45C explains the differences in bolded font/parenthesis/brackets/braces as related each Example's scores, and these visual features are for ease of association of a contemplated mechanism of action with examples producing more preferred scores.

TABLE 45C
Treatment Substances that Promote Ease of Ejaculation/Orgasm
Classification of: Treatment Substance\Treatment Substance\\Treatment Substance

| | | |
|---|---|---|
| C Score | A/B Score | |
| Example* | Example** | Treatment Substance |

Positive Cholinergic

| | | |
|---|---|---|
| [T41:140] | T41:140 | Galantamine HBr |
| T36:256 | T36:256 | Citicoline |

Negative Monoaminergic

| | | |
|---|---|---|
| T36:278 | T36:278 | Sarpagandha |

Promotes Ketogenic Metabolic Processes

| | | |
|---|---|---|
| T41:8 | T41:8 | 60% Caprylic Acid |

Negative Adenosinergic

| | | |
|---|---|---|
| T41:10 | T41:10 | 99% Theobromine |

Positive Adenosinergic

| | | |
|---|---|---|
| T41:2 | T41:2 | Incarvillea sinensis |

Positive GABAergic

| | | |
|---|---|---|
| T38:5 | [T38:5] | Homotaurine |
| T36:3 | T36:3 | MagT, Taurine |
| T41:3 | T41:3 | Gotu Kola |

Positive GABAergic\Other Treatment Substance

| | | |
|---|---|---|
| T38:14 | [T38:14] | NGABA\Collagen Peptides |
| T36:4 | T36:4 | MagT\Acetyl-Carnitine HCl |
| T36:217 | T36:217 | Val, MagT\5HTP |

Positive GABAergic\Positive Cysteineic

| | | |
|---|---|---|
| T39:14A | [T39:14A] | Val, 95% PiperineC\NAC |
| T37:73 | T37:73 | NGABA, MagT, Boswellia\NAC |
| T37:72 | T37:72 | Val, MagT, Boswellia\NAC |
| T36:151 | [T36:151] | NGABA, 95% Baicalin\NAC |

Positive GABAergic\Positive Cysteineic\\Other Treatment Substance

| | | |
|---|---|---|
| {[T36:218]} | [T36:218] | Val, MagT\NAC\\5HTP |
| T36:253 | [T36:253] | NGABA, Saffron Extract\NAC\\Collagen Peptides |
| T37:81 | [T37:81] | Val\NAC\\Pramiracetam |
| T41:135 | T41:135 | Val\NAC\\Turkey Rhubarb |
| T41:137 | T41:137 | Val\NAC \\D-Beta-Hydroxybutyrate Ester, Potassium |

Positive GABAergic\Positive Cholinergic\\Another Treatment Substance

| | | |
|---|---|---|
| T36:199 | [T36:199] | NGABA\Huperzine A\\Calcium Pyruvate |

Positive GABAergic\Positive Cysteineic\\Positive Cholinergic

| | | |
|---|---|---|
| T37:39 | [T37:39] | NGABA, MagT\NAC\\Galantamine HBr |
| T36:268 | [T36:268] | Val, MagT\NAC\\Galantamine HBr |
| T36:270 | T36:270 | Val, MagT\NAC\\Galantamine HBr |

Positive GABAergic\Positive Cysteineic\\Positive
Cholinergic\\\Another Treatment Substance

| | | |
|---|---|---|
| T36:271 | {[T36:271]} | Val, MagT\NAC\\Galantamine HBr\\\D-Ser, DimethylGly |
| T37:42 | T37:42 | NGABA, MagT\NAC\\Galantamine HBr\\\D-Ser, |
| | | DimethylGly |
| [T37:43] | T37:43 | NGABA, MagT\NAC\\Galantamine HBr\\\D-Ser, |
| | | DimethylGly, 5HTP |
| T37:46 | [T37:46] | Val, MagT, 50% Trans-Resveratrol\NAC\\Galantamine HBr, |
| | | Huperzine A\\\D-Ser, DimethylGly |

Positive GABAergic\Positive Cysteineic\\Gly\\\Other Treatment Substance

| | | |
|---|---|---|
| T36:20 | T36:20 | NGABA, MagT\NAC\\Gly\\\Tyr |
| T37:10 | [T37:10] | Val, MagT\NAC\\Gly\\\Tyr |
| T36:208 | T36:208 | Ile, MagT, Ashwagandha\NAC\\Gly\\\5HTP |

*Examples where the C score is below 2 in regular, not bold, font; Examples where the C score is 2 or above and below 3 in Bold; Examples where the C score is 3 or above and below 4 [In Bold in Brackets]; Examples where the C score is 4 or above and below 5 {[In Bold in Double Brackets]}; Examples where the C score is 5 or above {[(In Bold in Triple Brackets)]}
**Examples with both A and B scores are below 9 in regular, not bold, font; Examples with an A or B score of 9 or above but below 10 are in Bold; Examples where both A and B scores are 9 or above and below 10 [In Bold in Brackets]; Examples with an A or B score of 10 or above {[In Bold in Double Brackets]}; Examples where both A and B scores are 10 or above {[(In Bold in Triple Brackets)]}.

A positive cholinergic generally promoted the ease of ejaculation/orgasm (the DE score) and/or strength of muscle contraction in the genitals such as during ejaculation for a male (the D score). For example, galantamine HBr (i.e., an inhibitor of acetylcholinesterase, see FIG. 16 and FIG. 17; an inhibitor of butyrylcholinesterase; a Nicotinic ACh-R PAM) generally had improved D/DE scores (See Examples T36:268; T36:270; T36:271; T37:37; T37:38; T37:39; T37:40; T37:41; T37:42; T37:43; T37:44; T37:45; T37:46; T37:47; T37:48; T37:49; and T38:2 vs T41:140, T36:117, and T36:113). For example, Huperzine A [i.e., an inhibitor of acetylcholinesterase, and an inhibitor of NMDA-R (Polyamine Site Antagonist)] also had Examples of improved D/DE scores (See Examples T36:199, T36:231, T36:236, T36:242, T37:45, T37:46, T37:47, T37:63, T37:64, T37:74, T37:75, T37:76, T37:77, T37:79, T37:85, and T37:86 vs T36:129; T36:112, T36:232, T36:233, T36:234, T36:235, T36:237, T36:238, T36:239, T36:240, T36:241, T36:277, and T37:78). Examples of Huperizine A ingestion often had increased or decreased strength of the muscle contractions during ejaculation for an average D score of 3 and also had a DE score of 1.9 in the Examples herein when not combined with at least one positive cholinergic of the group galantamine HBr, Panax ginseng, alpha-glycerophosphocholine, centrophenoxine, and citicoline. However, a lower dose of about 0.005 mg of Huperzine A may promote D/DE score improvement (see Example T36:199). Galantamine HBr had an average D score of about 3.6 and an average DE score of 2.4 when ingested without combining with another positive cholinergic of at least one of Huperzine A, HBr, Panax ginseng, alpha-glycerophosphocholine, centrophenoxine, and citicoline. Galantamine HBr, huperzine A, and some other treatment substances increase in DE score scores may peak after ingestion before 60 minutes, such as at about 40 minutes, based on Examples T41:140, T37:46, T37:81, T39:14A, T41:135, T41:137, T41:2, and T41:3. As galantamine is a nicotinic ACh-R PAM, and has more consistently improved D/DE scores relative to huperzine A, a nicotinic ACh-R activator positive cholinergic is preferred. It is contemplated that a positive cholinergic will promote ease of achieving ejaculation and/or orgasm, including in persons (e.g., men, women) lacking a sexual disfunction, particularly when combined with a positive GABAergic and/or other treatment substances (e.g., a positive Cysteineic, a negative Glutamatergic) described herein.

It was contemplated a treatment substance that reduces/opposes GABAergic neurotransmission when ingested with a positive GABAergic treatment substance may reduce the positive effects on a sexual function by the positive GABAergic treatment substance. Val and NAC, a positive GABAergic treatment substance in combination with a positive Cysteineic, improved sexual functions. Val and NAC were selected to be combined with a treatment substance that contemplated to reduce GABAergic neurotransmission. For example, one or more carbonic anhydrases that increase bicarbonate (HCO3−) amount inside a cell such as a neuron promote the activity of GABAA-R as Cl− outside of a neuron is exchanged for the bicarbonate (or at least bicarbonate generally moves opposite to the direction as Cl−) upon activation of GABAA-R (See FIG. 14 and FIG. 15, as depicted in the postsynaptic neuron, the GABAA-R and CAase II/VII). It was contemplated that carbonic anhydrase production of H+ inside a cell may also promote movement of Cl− into the cell. It was contemplated that inhibition of a carbonic anhydrase may reduce the movement of Cl− into a cell by an activated GABAA-R. Various carbonic anhydrase inhibitors were evaluated as treatment substances to inhibit the production of bicarbonate/H+, as it was contemplated that would reduce GABAA-R activity's positive effect on TSF and other sexual functions. It is also contemplated that an activator of carbonic anhydrase activity (e.g., inhaling air enriched with carbon dioxide between about 0.042% to about 5% and/or otherwise below a toxic level of carbon dioxide), may support and/or enhance GABAA-R activity and have a positive effect on TSF/sexual functions. It was contemplated that ingesting NMDA to activate the NMDA-R would reduce improvement in a sexual function by ingesting Val and NAC, as NMDA-R activation allows a positively charged ion (e.g., Na+) to move into a cell to oppose the effects of the GABAA-R moving a negatively charged ion (e.g., Cl−) into a cell.

Table 46A shows Examples where the time from ingestion of a treatment substance to measurement of the B score was 60 minutes. Early C score measurements, when available, are shown for 20, 30, 40, and/or 50 minutes. Examples having early C score measurements for Val and NAC and another treatment substance with a preferred C score of 2.0 or more and/or a more preferred A/B score of 9.0 or more are shown under “Examples of Val and NAC and Another Treatment Substance with a High C, A, and/or B Score.” These Examples have an average C score pattern that rises from about 20 minutes to peak at about 30 to about 40 minutes then declines at about 50 minutes (a “rise, peak, fall pattern”; See “Average” under “Average, Lowest and Highest Sexual Function Scores for Examples of Val and NAC and Another Treatment Substance With a High C, A, and/or B Score”). Individual combinations of Val and NAC and another treatment substance may vary from the average's rise, peak, fall pattern (e.g., T41:120). Though the average C score at 60 minutes was greater than at 50 minutes, this is contemplated as a result of having more C score measurements at 60 minutes and fewer C score measurements at the 20 to 50 minutes to include in the average for each timepoint; and that additional measurements may show a decline between 50 and 60 minutes.

Shown next at Table 46A are Examples of Val and NAC alone that also had a high C, A, and/or B score. Though early C scores at 20 to 50 minutes were not measured to evaluate the C value pattern for these examples, the average was a High C, A, and/or B Score (See “Average” row under “Average, Lowest and Highest Sexual Function Scores for Examples of Val and NAC Alone”).

Next at Table 46A are shown Examples of a combination of Val, NAC and a carbonic anhydrase inhibitor or NMDA, and these Examples produced an average C, A and/or B scores that were reduced at 60 minutes below preferred levels (See “Average” Row below “Average, Lowest and Highest Sexual Function Scores for Examples With a Carbonic Anhydrase Inhibitor Or a NMDA-R Activator”). The pattern for the average C scores differed from the rise, peak, fall pattern with the greatest average C score being at 20 minutes (measurements earlier than 20 minutes were generally not taken) and the average C scores consistently declined to the B score measurement at 60 minutes (a “decline pattern”). The Examples under “Examples of Val and NAC and a Carbonic Anhydrase Inhibitor” show a combination of Val and NAC and an individual carbonic anhydrase inhibitor, wherein some carbonic anhydrase inhibitors, hesperidin and 1% spermidine, had a rise, peak, fall pattern of C scores and not a high C, A, and/or B scores. Val and NAC in combination with chrysin, a carbonic anhydrase inhibitor formulated from the vendor with the possible positive GABAergic treatment substance piperine, had a C score pattern that resembled the rise, peak, fall pattern and a high C, A, and/or B score, and therefore Example T41:128 was categorized in the Examples of Val and NAC and another treatment substance with a high C, A, and/or B score. It is contemplated that the Examples with hesperidin, 1% spermidine, and a combination of chrysin and piperine may be indicative of the interaction of conflicting mechanisms of action of treatment substances counterbalancing each other. It was contemplated that using the decline pattern of C scores, a treatment substance may be identified that reduces a sexual function (e.g., TSF) alone or in combination with a treatment substance that improves a sexual function (e.g., Val and Cys). It is contemplated that identifying the treatment substance that reduces a sexual function may be used to identify another treatment substance that has the similar effects by having the same/similar or different mechanism of action. For example, the decline pattern in C score pattern was used to identify other treatment substances that were not selected for evaluation as a carbonic anhydrase inhibitors or NMDA-R activator such as (See “Examples of Treatment Substances Where The C Score Declined in a Similar Pattern as for Val and NAC and a Carbonic Anhydrase Inhibitor Or a NMDA-R Activator”), and each such treatment substance is referred to herein as a “C score inhibiting treatment substance” that may reduce other sexual scores and sexual functions. For example, at 60 minutes, Val and NAC combined with either bahera, turkey rhubarb, alpha-glycosyl isoquercitrin, or a combination of D-beta-hydroxybutyrate ester and potassium had reduced C, A and B scores to below preferred levels; Val and NAC combined with either Ginkgo or dihydromyricetin had C, A and B scores less than the lowest C, A and B scores of Val and NAC alone; and Val, NAC and icariin had a C score below a preferred C score and A and B scores that matched the lowest A and B scores of Val and NAC alone. The average of all the identified C score inhibiting treatment substances with Val and NAC is shown at the bottom of Table 46A, and shows the decline pattern. Of note, the scores for an individual treatment substance may not follow the pattern observed when combined with other treatment substances such as Val and NAC, as the average of all C score inhibiting treatment substances when ingested alone (i.e., apigenin at Examples T41:81 and T41:82, hesperidin at Examples T41:88 and T41:89, etc.) had a rise, peak, fall pattern but with the peak at 50 minutes and a decline at 60 minutes, however bahera (Examples T41:44 and T41:43) and dihydromyricetin (Example T41:42 at a higher dose than Example T41:22) alone also had the decline pattern. For example, descriptions of the mechanisms of action of a treatment substance that is a carbonic anhydrase inhibitor and/or a C score inhibiting treatment substance are shown at Table 46B3; and it is contemplated that other treatment substances that have the same or similar mechanisms of action, including other known mechanisms of action not listed herein, may also reduce sexual functions (e.g., TSF), particularly when combined with a treatment substance that improves a sexual function.

TABLE 46A
Change of C Score Over Time

| | C Scores at Different | | | | | | | |
|---|---|---|---|---|---|---|---|---|
| | Times in Minutes (“Min”) | | | | | | A and B | |
| | 20 | 30 | 40 | 50 | 60 | | Scores | |
| Example | Min | Min | Min | Min | Min | Treatment Substance | A | B |

Examples of Val (and Other Positive GABAergic) and\NAC\\and
Optionally Another Treatment Substance With a High C, A, and/or B Score

| | | | | | | | | |
|---|---|---|---|---|---|---|---|---|
| T36: 268 | | 2.0 | | | 2.0 | Val, MagT\NAC\\ | 9.5 | 9.5 |
| | | | | | | Galantamine HBr | | |
| T37: 15 | | 2.5 | | | 2.0 | Val, MagT\NAC\\Oleamide | 9.5 | 9.8 |
| T37: 16 | | 2.0 | | | 1.5 | Val, MagT\NAC\\Oleamide | 9.5 | 8.5 |
| T37: 19 | | 1.8 | | | 1.2 | Val, Ile, Leu, MagT\NAC\\ | 8.8 | 9.5 |
| | | | | | | Riboflavin, Pyridoxine | | |
| T37: 24 | | | 2.2 | | 2.2 | Val, NGABA, MagT\NAC | 9.5 | 9.8 |
| T37: 26 | | 1.8 | | | 1.8 | Val\NAC, Cys | 9.5 | 9.5 |
| T37: 34 | | | 2.0 | | 2.0 | Val, MagT, Theanine\NAC\\ | 9.5 | 9.5 |
| | | | | | | Inositol | | |
| T37: 36 | | | 2.2 | | 1.8 | Val, MagT\NAC\\D-Ser, | 9.5 | 9.8 |
| | | | | | | DimethylGly | | |
| T37: 41 | | 2.2 | | | 1.8 | Val, MagT\NAC\\ | 9.8 | 9.8 |
| | | | | | | Galantamine HBr | | |
| T37: 45 | | | 1.8 | | 2.0 | Val, MagT\NAC\\ | 9.2 | 9.2 |
| | | | | | | Galantamine HBr, | | |
| | | | | | | Huperzine A, DimethylGly | | |
| T37: 48 | | | 2.0 | | 1.2 | Val, MagT\NAC\\ | 9.5 | 9.5 |
| | | | | | | Galantamine HBr, D-Ser, | | |
| | | | | | | DimethylGly, Alpha-Lipoic | | |
| | | | | | | Acid, Acetyl-Carnitine, | | |
| | | | | | | Ubiquinone | | |
| T37: 49 | | | 2.2 | | 2.2 | Val, MagT\NAC\\ | 9.2 | 9.2 |
| | | | | | | Galantamine HBr, | | |
| | | | | | | D-Ser, Inosine | | |
| T37: 51 | | | 1.8 | | 1.5 | Val, MagT, 95% | 9.2 | 9.2 |
| | | | | | | Baicalin\NAC | | |
| T37: 58 | | | 1.8 | | 1.5 | Val\NAC\\Norvaline | 9.5 | 9.5 |
| T37: 59 | | | 1.8 | | 1.5 | Val, MagT\NAC\\ | 9.2 | 9.2 |
| | | | | | | DimethylGly, Sarcosine, | | |
| | | | | | | Betaine | | |
| T37: 60 | | | 1.8 | | 1.5 | Val, MagT\NAC\\Norvaline | 9.5 | 9.5 |
| T37: 61 | | | 1.8 | | 1.2 | Val\NAC\\Gly | 9.2 | 9.2 |
| T37: 63 | | | 1.5 | | 1.0 | Val, MagT, Saffron Extract, | 9.5 | 9.5 |
| | | | | | | Kava\NAC\\Huperzine A, | | |
| | | | | | | Calcium Pyruvate | | |
| T37: 72 | | 2.5 | | | 2.0 | Val, MagT, Boswellia\NAC | 8.5 | 9.5 |
| T37: 77 | | | 2.0 | | 1.0 | Val, MagT, Saffron Extract, | 8.8 | 9.2 |
| | | | | | | Kava\NAC\\Huperzine A, | | |
| | | | | | | Beta-Ala | | |
| T40: 10 | | | 1.8 | | 1.8 | Val, MagT\NAC\\Gly | 9.5 | 10.5 |
| T40: 13 | | | | 1.8 | 1.2 | Val\NAC\\Pramiracetam | 10.2 | 10.2 |
| T41: 111 | 0.5 | 1.0 | 1.5 | 1.5 | 1.5 | Val, Valerian Root Extract, | 9.2 | 0.0 |
| | | | | | | Valerian Root\NAC | | |
| T41: 112 | 1.2 | 1.8 | 2.0 | 1.2 | 1.2 | Val, Lemon Balm | 9.2 | 8.8 |
| | | | | | | Extract\NAC | | |
| T41: 113 | 1.2 | 1.5 | 2.0 | 2.0 | 1.5 | Val\NAC\\99% | 9.2 | 9.2 |
| | | | | | | Theobromine | | |
| T41: 120 | 2.0 | 1.2 | 1.0 | 1.2 | 1.2 | Val\NAC\\Cloves Extract | 9.2 | 8.8 |
| T41: 128 | 1.0 | 1.8 | 0.8 | 1.0 | 1.0 | Val, 95% PiperineS\NAC\\ | 9.2 | 8.5 |
| | | | | | | Chrysin | | |
| T41: 138 | 1.0 | 1.5 | 1.5 | 0.5 | 0.2 | Val\NAC\\Ornithine | 9.2 | 9.2 |

Average, Lowest and Highest Sexual Function Scores for Examples of Val (and Other
Positive GABAergic) and\NAC and\\Optionally Another Treatment Substance with a
High C, A, and/or B Score

| | | | | | | | |
|---|---|---|---|---|---|---|---|
| Average | 1.2 | 1.8 | 1.8 | 1.3 | 1.5 | 9.3 | 9.1 |
| Lowest | 0.5 | 1.0 | 0.8 | 0.5 | 0.2 | 8.5 | 0.0 |
| Highest | 2.0 | 2.5 | 2.2 | 2.0 | 2.2 | 10.2 | 10.5 |

Examples of Val and\NAC Alone

| | | | | |
|---|---|---|---|---|
| T36: 35 | 2.2 | Val\NAC | 9.5 | 8.8 |
| T36: 43 | 2.0 | Val\NAC | 9.5 | 9.5 |
| T36: 44 | 2.5 | Val\NAC | 9.5 | 8.8 |
| T37: 67 | 4.0 | Val\NAC | 9.5 | 9.5 |
| T38: 8 | 1.5 | Val\NAC | 9.2 | 9.2 |

Average, Lowest and Highest Sexual Function Scores for Examples of Val and\NAC Alone

| | | | |
|---|---|---|---|
| Average | 2.4 | 9.4 | 9.2 |
| Lowest | 1.5 | 9.2 | 8.8 |
| Highest | 4.0 | 9.5 | 9.5 |

Examples of Val and\NAC and\\Carbonic Anhydrase Inhibitor

| | | | | | | | | |
|---|---|---|---|---|---|---|---|---|
| T41: 127 | 1.2 | 1.2 | 1.0 | 0.5 | 0.0 | Val\NAC\\Apigenin | 4.5 | 4.5 |
| T41: 129 | 0.5 | 0.8 | 0.2 | 0.0 | 0.0 | Val\NAC\\Hesperidin | 5.5 | 0.0 |
| T41: 130 | 1.0 | 0.5 | 0.0 | 0.2 | 0.0 | Val\NAC\\80% Luteolin | 4.5 | 4.5 |
| T41: 131 | 1.5 | 1.2 | 1.0 | 0.8 | 0.0 | Val\NAC\\Mangosteen | 7.5 | 5.5 |
| T41: 139 | 0.5 | 0.5 | 1.2 | 0.5 | 0.5 | Val\NAC\\1% Spermidine | 0.0 | 0.0 |

Examples of Val and\NAC and\\NMDA-R Activator

| | | | | | | | | |
|---|---|---|---|---|---|---|---|---|
| T41: 126 | 0.8 | 0.8 | 0.5 | 0.2 | 0.0 | Val\NAC\\NMDA | 3.5 | 3.5 |

Average, Lowest and Highest Sexual Function Scores for Examples
With a Carbonic Anhydrase Inhibitor Or a NMDA-R Activator

| | | | | | | | |
|---|---|---|---|---|---|---|---|
| Average | 0.9 | 0.8 | 0.7 | 0.4 | 0.1 | 4.3 | 3.0 |
| Lowest | 0.5 | 0.5 | 0.0 | 0.0 | 0.0 | 0.0 | 0.0 |
| Highest | 1.5 | 1.2 | 1.2 | 0.8 | 0.5 | 7.5 | 5.5 |

Examples of Treatment Substances where the C Score Declined in a Similar Pattern
as for Val and\NAC and\\a Carbonic Anhydrase Inhibitor Or a NMDA-R Activator
Examples of Val and\NAC and\\Nitric Oxide Inhibitor

| | | | | | | | | |
|---|---|---|---|---|---|---|---|---|
| T41: 119 | 0.8 | 0.0 | 0.0 | 0.2 | 0.0 | Val\NAC\\Bahera | 2.5 | 0.0 |

Examples of Val and\NAC and\\OAT 1/3 Inhibitor

| | | | | | | | | |
|---|---|---|---|---|---|---|---|---|
| T41: 135 | 1.8 | 0.0 | 0.0 | 0.5 | 0.2 | Val\NAC\\Turkey Rhubarb | 6.5 | 5.5 |

Examples of Val and\NAC and\\OAT 1/3 Inhibitor/GABAA-R NAM

| | | | | | | | | |
|---|---|---|---|---|---|---|---|---|
| T41: 122 | 1.8 | 1.5 | 1.2 | 1.2 | 0.8 | Val\NAC\\Ginkgo | 8.2 | 7.5 |

Examples of Val and\NAC and\\Inhibitor of a GABAA-R PAM

| | | | | | | | | |
|---|---|---|---|---|---|---|---|---|
| T41: 143 | 2.0 | 1.2 | 0.5 | 0.2 | 0.2 | Val\NAC\\Dihydromyricetin | 8.2 | 7.5 |

Examples of Val and\NAC and\\Possible P-GP Inhibitor

| | | | | | | | | |
|---|---|---|---|---|---|---|---|---|
| T41: 136 | 1.2 | 0.8 | 0.2 | 0.0 | 0.0 | Val\NAC\\Alpha-Glycosyl | 3.5 | 3.5 |
| | | | | | | Isoquercitrin | | |

Examples of Val and\NAC and\\Negative Glutamatergic/Positive Adenosinergic

| | | | | | | | | |
|---|---|---|---|---|---|---|---|---|
| T41: 137 | 1.5 | 1.5 | 1.0 | 1.0 | 1.0 | Val\NAC\\ | 7.5 | 7.5 |
| | | | | | | D-Beta-Hydroxybutyrate | | |
| | | | | | | Ester, Potassium | | |

Examples of Val and\NAC and\\Positive Vasodilator

| | | | | | | | |
|---|---|---|---|---|---|---|---|
| T37: 82 | 1.5 | 1.0 | 0.5 | 0.2 | Val\NAC\\Icariin | 9.2 | 8.8 |

Average, Lowest and Highest Sexual Function Scores for Examples With a All C Score Inhibiting
Treatment Substances Listed Above Beginning With the Carbonic Anhydrase Inhibitors

| | | | | | | | |
|---|---|---|---|---|---|---|---|
| Average | 1.2 | 0.8 | 0.6 | 0.4 | 0.2 | 5.5 | 4.5 |
| Lowest | 0.5 | 0.0 | 0.0 | 0.0 | 0.0 | 0.0 | 0.0 |
| Highest | 2.0 | 1.5 | 1.2 | 1.2 | 1.0 | 9.2 | 8.8 |

TABLE 46B
Treatment Substances in Examples That Are Carbonic Anhydrase
Inhibitors and/or a C Score Inhibiting Treatment Substance

| Treatment | |
|---|---|
| Substance | Classification(s) [Detail] |
| Apigenin | Negative/Positive GABAergic [Carbonic Anhydrase I/II/V/IX/XIV |
| | Inhibitor, GABAA-R Having Alpha1/Beta1/Gamma2S Subunit Antagonist; |
| | GABAARho-R Antagonist; Glutamate Decarboxylase Inhibitor; |
| | GABAA-R Benzodiazepine Site PAM]; Transporter Inhibitor |
| | [BCRP/MRP1/MCT1 Inhibitor)]; Other Active: Alpha-Bisabolol |
| Hesperidin | Negative GABAergic [Carbonic Anhydrase I/II/V/VII/VIII/XII |
| | Inhibitor]; Transporter Inhibitor [MCT1 Inhibitor] |
| Luteolin | Negative/Positive GABAergic [Active: Luteolin (Carbonic Anhydrase V/ |
| Preparation | VII Inhibitor; Glutamate Decarboxylase Inhibitor; GABAA-R PAM)]; |
| | Transporter Inhibitor [Active: Luteolin (BCRP/MRP1/MCT1 Inhibitor)]; |
| | Negative Dopaminergic/Noradrenalinergic [Active: Luteolin (DAT |
| | Activator)] |
| Mangosteen | Possible Negative GABAergic [Active: Alpha-Mangostin (Possible |
| | Carbonic Anhydrase VA Inhibitor)] |
| Spermidine | Positive/Negative Glutamatergic [Active: Spermidine (NMDA-R |
| Preparation | Polyamine Site Agonist; Channel Blocker for NMDA-R with NR2A/NR2B |
| | Subunit But PAM when Gly Concentration Low; |
| | AMPA-R/Kainate-R/mGlu6-R Channel Blocker)]; Negative/Positive |
| | GABAergic [Active: Spermidine (Carbonic Anhydrase Type: I/II/III/IV/ |
| | V/VI/VII/VIII/IX/X /XI/XII/XIV Inhibitor by Binding Active |
| | Site Water); GABA Precursor (In Polyamine Metabolism); TRPV1-R |
| | Activator (At High Concentration)]; Positive Cannabinoidergic [Active: |
| | Spermidine (N-Acylphosphatidylethanolamine-Hydrolyzing Phospholipase |
| | D Activator; Increases Spermidine/Spermine N1-Acetyltransferase |
| | Amount)]; Positive GHBergic [Active: Spermidine (GHB Precursor)]; |
| | Positive/Negative Polyaminergic [Active: Spermidine |
| | (Spermine/Putrescine Precursor; Promotes Ornithine Decarboxylase |
| | Degradation/Reduces SAMe Decarboxylase Amount)]; Active: Spermidine |
| | (Beta-Ala Precursor; See Beta-Ala Herein) |
| NMDA | Positive/Negative Glutamatergic [NMDA-R Glu Site Agonist (Synaptic |
| | NMDA-R Activation Promotes Glutamatergic Neurotransmission, but Also |
| | Reduces AMPA-R Amount)]; Negative GABAergic [NMDA-R Activation |
| | (Decreases KCC2 Phosphorylation that Decreases KCC2 Activity and Cell |
| | Surface Amount and Increases KCC2 Protein Degradation)]; Positive |
| | Cannabinoidergic [Synaptic NMDA-R Activation (Promotes |
| | N-Acylphosphatidylethanolamine-Hydrolyzing Phospholipase |
| | D/Diacylglycerol Lipase-Alpha Activation)]; Neurotransmitter Release |
| | Activator/Inhibitor [Activation of Extrasynaptic NMDA-R on a Presynaptic |
| | Neuron (Generally Increases Release of Neurotransmitters but Activation |
| | with CB1-R May Inhibit Neurotransmitter Release)]; Transporter Activator |
| | [Activation of NMDA-R (Increases Arachidonic Acid Inside BBB cell that |
| | Increases P-GP Amount)] |
| Bahera | Negative GABAergic [Active: Gallic Acid, Ellagic Acid (Carbonic |
| | Anhydrase Type I/II/III/IV/V/VI/VII/VIII/IX/XIV Inhibitor)]; |
| | Negative Nitroergic [Active: Chebulagic Acid, Chebulinic Acid, Gallic |
| | Acid, Ellagic Acid (Nitric Oxide Synthetase Inhibitor)]; Transporter |
| | Inhibitor [Active: Gallic Acid (OAT1/3 Inhibitor)] |
| Turkey Rhubarb | Transporter Inhibitor [Active: Rhein, Chrysophanol, Physcion |
| | (OAT1/OAT3 Inhibitor)]; Other Active: Emodin, Aloeemodin, |
| | Anthraquinone |
| Ginkgo | Negative GABAergic [Active: Bilobalide (GABAA-R/GABAARho-R |
| | NAM)]; Negative Glycinergic [Active: Bilobalide, Ginkgolide (e.g., |
| | Ginkgolide A/B/C/J/M) (Gly-R NAM)]; Positive Glutamatergic [mGlu8-R |
| | Agonist]; Positive Noradrenalinergic [Active: Glycoside, Ginkgolide A |
| | (NET Inhibitor)]; Transporter Inhibitor [Active: 17:1 Ginkgolic Acid |
| | (OAT1 Inhibitor); Active: 15:1 Ginkgolic Acid, 17:1 Ginkgolic Acid |
| | (OAT3 Inhibitor); Active: Ginkgolide B (P-GP Inhibitor/Reduces P-GP |
| | Amount); Active: Ginkgo Biloba Preparation (Decreased MRP1 Amount)]; |
| | Other Active: Quercetin, Kaempferol, Isorhamnetin |
| Dihydromyricetin | Negative GABAergic [GABAA-R Alcohol Site Antagonist/NAM (Inhibits |
| | Alcohol's GABAA-R PAM Activity)] |
| Alpha-Glycosyl | Possible Transporter Inhibitor [Possible P-GP Inhibitor as Chemically |
| Isoquercitrin | Similar to Isoquercitrin] |
| D-Beta-Hydroxyb | Negative Glutamatergic [Active: Beta-Hydroxybutyrate (VGLUT2 |
| utyrate Ester, | Inhibitor)]; Positive Adenosinergic [Active: Beta-Hydroxybutyrate |
| Potassium | (Promotes Ketogenic Metabolic Processess) (See 55% Caprylic Acid and |
| | 35% Capric Acid Herein)] |
| Icariin Preparation | See Table 45A |

It is contemplated that the method of measuring C, A, B, and/or other sexual function scores over time (e.g., about 0 minutes to about 3000 minutes or more after ingesting a treatment substance) for a rise, peak, fall pattern, a decline pattern, or other pattern (e.g., “fall then rise pattern,” see example T41:120) may be used to identify a treatment substance/target of a treatment substance/mechanism of action. It is contemplated that identifying the mechanism of action of a treatment substance that affects a sexual function (e.g., a treatment substance that reduces TSF) may be used to identify a treatment substance that generally produces the opposite effect for a sexual function (e.g., a treatment substance that improves TSF) by preferentially having the opposite mechanism of action. For example, various negative glutamatergic treatment substances (e.g., MagT, NAC/Cys, saffron, cat's claw bark/cat's claw bark extract, pyroglutamate, etc.), particularly a negative NMDA-R glutamatergic treatment substance as described herein, have improved TSF C/A/B scores particularly when in combination with at least a positive GABAergic treatment substance; and it is contemplated that as NMDA is an agonist for the Glu site of an NMDA-R and that NMDA produced a negative effect on TSF scores, that a treatment substance that has antagonist activity for the NMDA-R Glu site would produced a positive effect for TSF scores, particularly in combination with a positive GABAergic/positive cysteineic/other treatment substance described herein. In another example, the treatment substance dihydromyricetin has a mechanism of action as an antagonist of the GABAA-R alcohol PAM/anesthetic site. The C value decline pattern of dihydromyricetin alone and in combination with Val and NAC is contemplated as being exemplary of the method described herein of identifying a treatment substance that reduces a sexual function (e.g., a treatment substance that is an antagonist of the GABAA-R alcohol PAM/anesthetic site) to help identify a treatment substance that preferentially has the opposite mechanism of action (e.g., a GABAA-R activator) that may improve a sexual function. In a particular example, as dihydromyricetin reduced positive TSF scores as an antagonist of the GABAA-R alcohol PAM/anesthetic site in the absence of the inventor being intoxicated, it is contemplated that a PAM other than alcohol may be naturally produced/present that may be activating the GABAA-R at the alcohol site, and this PAM and other treatment substances that act on the alcohol site as a PAM for the GABAA-R would have a positive effect on TSF scores. It is also contemplated that a treatment substance that reduces sexual functions such as TSF may be used to improve certain other sexual functions such as reducing the incidence of premature ejaculation, and to identify other treatment substance(s).

It is contemplated that selection of treatment substances that differentiate between activation of species of proteins (e.g., neurotransmitter receptor subtypes, synaptic vs extrasynaptic receptors) may provide a method for selection of treatment substances for improved effects on sexual disfunctions. For example, the treatment substance phenibut is a full agonist for the metabotropic GABAB-R and a weak agonist for the GABAA-R, and phenibut produced a weaker effect on improving A/B TSF scores at a higher dose in milligrams than a partial agonist for the Cl− ion channel controlling GABAA-R (with mixed agonist/antagonist activity for the GABAB-R) (See Table 47A). It is contemplated that activation of a GABAA-R is preferred, particularly for the A/B value measurements, relative to activating a GABAB-R for improving TSF.

TABLE 47A
GABAB-R Agonist (Phenibut) vs. GABAA-R
Partial Agonist (Homotaurine)

| Example | Treatment Substance (Dose in mg) | C | A | B |
|---|---|---|---|---|
| T39:13 | Phenibut (450) | 1.5 | 6.5 | 6.5 |
| T38:18 | Homotaurine (50) | 1.0 | 9.2 | 9.2 |
| T38:5 | Homotaurine (150) | 1.8 | 9.5 | 9.5 |
| T37:57 | Homotaurine (200) | 1.2 | 9.2 | 9.2 |
| T37:56 | Homotaurine (250) | 1.2 | 7.5 | 7.5 |

It is contemplated that selection of a treatment substance that crosses the BBB more readily than another treatment substance with like activity may provide a method for selection of treatment substance(s) for improved effects on sexual disfunctions depending upon ease of crossing the BBB. For example, a positive GABAergic, Val/NGABA, that readily crosses the BBB improved in combination with a positive Cysteineic/GSHic (e.g., NAC) that also crosses the BBB had preferred TSF scores (e.g., a C/A/B score) more consistently than when combined with a positive Cysteineic/GSHic (e.g., Cys/GSH) that less readily crosses the BBB (See Table 47B). It is contemplated that a treatment substance that crosses the BBB is a preferred relative to treatment substance with like activity that less readily crosses the BBB.

TABLE 47B
NAC in Combination with a Positive GABAergic (Val or NGABA)

| | | | | |
|---|---|---|---|---|
| Example | Treatment Substance (Dose in mg) | C | A | B |

Val

| | | | | |
|---|---|---|---|---|
| T36:87 | Val (3600) | 1.8 | 7.8 | 7.8 |

NAC

| | | | | |
|---|---|---|---|---|
| T36:36 | NAC.SR (1200) | 0.5 | 1.5 | 1.5 |

Val and\Positive Cysteineic That Less
Readily Crosses the BBB Than NAC

| | | | | |
|---|---|---|---|---|
| T36:96 | Val (3600)\GSH (1000) | 1.2 | 6.5 | 6.5 |
| T36:122 | Val (3600)\Cys (1000) | 1.2 | 7.8 | 7.8 |

Val and\NAC and Cys

| | | | | |
|---|---|---|---|---|
| T37:26 | Val (3600)\NAC.P (500), Cys (500) | 1.8 | 9.5 | 9.5 |

Val and\NAC

| | | | | |
|---|---|---|---|---|
| T36:44 | Val (3600)\NAC.P (250) | 2.5 | 9.5 | 8.8 |
| T36:43 | Val (3600)\NAC.P (500) | 2.0 | 9.5 | 9.5 |
| T38:8 | Val (4320)\NAC.P (500) | 1.5 | 9.2 | 9.2 |
| T37:67 | Val (3150)\NAC.P (1000) | 4.0 | 9.5 | 9.5 |
| T36:35 | Val (3150)\NAC.P (500), NAC.SR (600) | 2.2 | 9.5 | 8.8 |

NGABA

| | | | | |
|---|---|---|---|---|
| T36:61 | NGABA (200) | 1.0 | 6.5 | 7.5 |

NGABA and\Positive Cysteineic That Less
Readily Crosses the BBB Than NAC

| | | | | |
|---|---|---|---|---|
| T36:105 | NGABA (200)\Cys (1000) | 1.8 | 8.8 | 8.5 |
| T36:75 | NGABA (200)\GSH (1000) | 1.2 | 7.5 | 7.5 |

NGABA and\NAC

| | | | | |
|---|---|---|---|---|
| T36:46 | NGABA (200)\NAC.P (500) | 1.2 | 8.5 | 7.5 |
| T36:69 | NGABA (200)\NAC.P (500) | 1.8 | 8.5 | 9.5 |
| T36:45 | NGABA (200)\NAC.P (1000) | 1.5 | 8.5 | 6.8 |
| T36:50 | NGABA (200)\NAC.P (1500) | 1.8 | 8.5 | 8.8 |
| T39:16 | NGABA (500)\NAC.P (500) | 2.0 | 9.5 | 9.2 |

Table 47C shows average, lowest and highest summary statistic scores from various working examples herein and are exemplary of the improvement in sexual function scores by the treatment substance(s) described herein. Shown first are the scores from the examples listed at Table 35 wherein no treatment substance was ingested. The scores from the examples listed at Table 45B having treatment substances producing preferred C, A or B scores is demonstrative of the positive effects of selected treatment substances described herein. The scores from the examples listed at Table 40C is demonstrative of the positive effect of multiple doses of selected treatment substances described herein, with indications of improved C and A scores relative to Table 45B. The scores from the examples listed at Table 45C focused on treatment substances Promoting Ejaculation/Orgasm (higher D/DE scores). The summary scores for working Examples T36:268, T36:270, T36:271, T37:37, T37:38, T37:39, T37:40, T37:41, T37:42, T37:43, T37:44, T37:45, T37:46, T37:47, T37:48, T37:49, T38:2 that comprise the treatment substances Val/NGABA and NAC and Galantamine are exemplary of the improvements of various sexual function scores, C, A, B, D and DE, by a combination of a positive GABAergic, a positive Cysteineic, and a positive Cholinergic.

TABLE 47C
Summary Statistics of Average, Lowest and Highest Sexual Function Scores

| | | | | | | | | |
|---|---|---|---|---|---|---|---|---|
| Statistics | C | CT | A | B | BM | BS | D | DE |

No Treatment Substance Ingested Examples from Table 35

| | | | | | | | | |
|---|---|---|---|---|---|---|---|---|
| Average | 0.2 | N/A | 2.4 | 2.6 | 1.2 | N/A | 2.9 | 2.0 |
| Lowest | 0.0 | N/A | 0.0 | 0.0 | 0.0 | N/A | 1.0 | 1.0 |
| Highest | 0.8 | N/A | 8.8 | 8.8 | 8.8 | N/A | 3.0 | 2.0 |

Treatment Substance Examples With a
Preferred C, A and/or B Score from Table 45B

| | | | | | | | | |
|---|---|---|---|---|---|---|---|---|
| Average | 1.8 | N/A | 9.1 | 9.2 | 5.2 | N/A | 3.2 | 2.1 |
| Lowest | 0.2 | N/A | 6.5 | 0.0 | 0.0 | N/A | 0.0 | 0.0 |
| Highest | 5.0 | N/A | 10.5 | 10.5 | 9.5 | N/A | 5.0 | 3.0 |

Multiple Doses of One or More Treatment Substance Examples from Table 40C

| | | | | | | | | |
|---|---|---|---|---|---|---|---|---|
| Average | 2.0 | N/A | 9.5 | 9.2 | 0.0 | N/A | 3.1 | 1.9 |
| Lowest | 1.2 | N/A | 8.5 | 0.0 | 0.0 | N/A | 0.0 | 0.0 |
| Highest | 3.5 | N/A | 10.5 | 10.5 | 0.0 | N/A | 4.0 | 2.0 |

Treatment Substance Examples Promoting Ejaculation/Orgasm from Table 45C

| | | | | | | | | |
|---|---|---|---|---|---|---|---|---|
| Average | 1.5 | N/A | 7.6 | 7.9 | 5.6 | N/A | 3.3 | 2.9 |
| Lowest | 0.0 | N/A | 0.0 | 0.0 | 0.0 | N/A | 2.5 | 2.0 |
| Highest | 4.0 | N/A | 9.5 | 10.5 | 9.5 | N/A | 5.0 | 3.0 |

Treatment Substance Examples Comprising Val/NGABA and NAC and Galantamine

| | | | | | | | | |
|---|---|---|---|---|---|---|---|---|
| Average | 1.8 | N/A | 9.3 | 9.3 | 5.9 | N/A | 4.0 | 2.4 |
| Lowest | 1.0 | N/A | 8.8 | 8.5 | 0.0 | N/A | 2.5 | 2.0 |
| Highest | 2.5 | N/A | 9.8 | 10.5 | 9.5 | N/A | 5.0 | 3.0 |

Many treatment substances, typically when ingested alone, often produced a sexual sensation A score and/or B score less than 9, and it is contemplated that a treatment substance that affects one neurotransmitter system that is not dominant in contributing to TSF and/or another sexual function (“nondominant”) may affect a neurotransmitter system that is more dominant in contributing to TSF and/or another sexual function (“dominant”). It is contemplated that mechanisms such as nondominant and dominant neurotransmitter corelease/cotransmission, and/or an alteration (e.g., promoted neurotransmission signaling, reduced neurotransmitter signaling) of the nondominant neurotransmitter system being communicated to a dominant neurotransmitter system to alter the dominant neurotransmitter system's activity (e.g., promoted dominant neurotransmitter signaling, reduced dominant neurotransmitter signaling) to affect a sexual function. It is contemplated that the positive GABAergic/positive cholinergic/negative glutamatergic neurotransmitter system are dominant for sexual functions (e.g., TSF, ease of ejaculator/orgasmic response), and it is contemplated that a positive cysteineic/positive GSHic may be promoting the activity of a positive GABAergic/positive cholinergic/negative glutamatergic neurotransmitter system.

A preferred treatment substance is a positive GABAergic substance that promotes GABAergic neurotransmission signaling (e.g., synaptic signaling), with examples including, a positive GABAic substance that promotes an increase of GABA in the body (e.g., a GABA precursor/prodrug), a GABA-R activator such as an agonist (e.g., a full agonist, a partial agonist), a GABA-R positive allosteric modulator (“PAM”), a substance that promotes an increased amount of a GABA-R on the surface of a cell; a substance that promotes ion channel opening/closing that promotes GABAergic signaling (e.g., an ion channel may increase cell membrane hyperpolarization, and another ion channel may then promote depolarization so a GABAergic signal can again be transmitted by repolarization); a substance that promotes GABA release into the intercellular (e.g., synaptic) space; a substance that reduces GABA degradation (e.g., a substance that inhibits an enzyme involved in GABA degradation); a substance that reduces movement of GABA from the extracellular space into a cell (e.g., a GABA reuptake inhibitor); a substance that promotes GABA synthesis (e.g., a substance that activates an enzyme involved in GABA synthesis); or combinations thereof. A preferred treatment substance is a positive GABAic substance that promotes an increase of GABA in the body (e.g., a GABA precursor/prodrug) and/or a GABA-R activator (e.g., a GABA-R agonist/GABA-R PAM). A GABAA-R/GABAARho-R activator is more preferred than a GABAB-R activator. Examples of a preferred GABA precursor/prodrug includes N-Nicotinoyl-GABA (“NGABA”), a BCAA (i.e., Val, Leu, Ile), or a combination thereof. Val is more preferred than Leu, and Leu more preferred than Ile. A preferred treatment substance is a positive Cysteineic/GSHic substance that promotes an increase of cysteine (“Cys”)/cystine (“Cys2”)/GSH in the body (e.g., a cysteine/cystine precursor/prodrug). A preferred positive Cysteineic/GSHic substance is N-acetyl-cysteine (“NAC”). A preferred combination of treatment substances is a positive GABAergic/GABAic substance that is a GABA-R activator, with a GABAA-R/GABAARho-R activator preferred, and a positive Cysteineic/GSHic substance. A GABAergic/GABAic substance and a positive Cysteineic/GSHic may be combined with other treatment substances described herein including but not limited to a substance that modifies (e.g., activates/inhibits) neurotransmitter signaling, via similar mechanisms as described for a substance that promotes GABAergic signaling except the targets and mechanisms of action are for a different neurotransmitter, with non-limiting examples including a substance that promotes cholinergic signaling (e.g., acetylcholine), dopaminergic signaling (e.g., dopamine), noradrenalinergic signaling (e.g., noradrenaline), adrenalinergic signaling (e.g., adrenaline), histaminergic signaling (e.g., histamine), melatoninergic signaling (e.g., melatonin), serotoninergic signaling (e.g., serotonin), glutamatergic signaling (e.g., Glu), D-Serinergic signaling (e.g., D-Ser), glycinergic signaling (e.g., Gly), agmatinergic signaling (e.g., agmatine), purinergic signaling (e.g., by ATP/ADP/adenosine/ketogenic metabolic processes), cannabinoidergic signaling (e.g., anandamide, 2-arachidonoyl glycerol), or a combination thereof. For example, a positive GABAergic substance/positive Cysteineic substance may be combined with a positive Cholinergic substance that promotes cholinergic signaling (e.g., an inhibitor of degradation of acetylcholine by cholinesterase) and/or a negative glutamatergic substance that reduces glutamatergic signaling (e.g., an NMDA-R inhibitor). In many embodiments, a preferred treatment substance has the ability to move across the BBB from the blood into the CNS (e.g., the brain) more readily than a treatment substance with the same or similar mechanism of action (e.g., an activator of a GABA-R) that has less ability to move across the BBB into the CNS. It is contemplated that other treatment substance(s) [e.g., a prescription drug, a different type of preparation of any biological material that have the same/similar mechanism of action (e.g., a positive GABAergic, a positive cholinergic, a negative glutamatergic, etc.)] as those described herein the working examples may be used to substitute for and/or be used with the treatment substance(s) of the working examples.

Most Examples selected a 60 minutes for a measurement of A/B/C or other score for consistency in comparison of each treatment substance's effects on a sexual function between Examples, and it is contemplated that optimization of time for measurement of each sexual function score for each individual ingesting a treatment substance can be readily deterimined by minor variations in time of measurement (e.g., about 5 to 100 minutes difference from the disclosures herein for many treatment substances). For example, it is contemplated that for a treatment substance, a peak C score (e.g, of about 30 to 40 minutes for Val and NAC) may be selected for measurement of an A/B/C or other sexual function score (e.g., Examples T39:14A and T39:15). It is further contemplated that optimization of time of measurement for each sexual function score for each individual will allow other compositions described herein as having an A/B score of between 7 and 9 (e.g., 8.5, 8.8) at 60 minutes will have a more preferred A/B scores of 9 or above (or different score of C, D, DE, etc.) at a different time than 60 minutes (e.g., between about 45 minutes earlier to about 45 minutes later; at 50 minutes, at 40 minutes, at 30 minutes, at 110 minutes, at 120 minutes, etc.) upon optimization in accordance with each individual's (e.g., a man, a woman) preference and/or a differing response to a treatment substance (e.g., due to genetic/phenotypic difference between individuals). Additionally, from early treatment substance evaluations the peak in the A score and B score may differ (e.g., be 20 minutes later) than the peak in C score, and it is contemplated each individual ingesting one or more treatment substances may optimize the time of TSF A score and B score generation according to personal preference in light of variations that may occur in each individual's response (e.g., genetic and/or phenotypic differences between individuals' transporters, enzymes, receptors) to the ingested treatment substance(s). For example, it is contemplated that individuals may or may not have the same and/or different sexual and/or non-sexual side effects when ingesting one or more treatment substances.

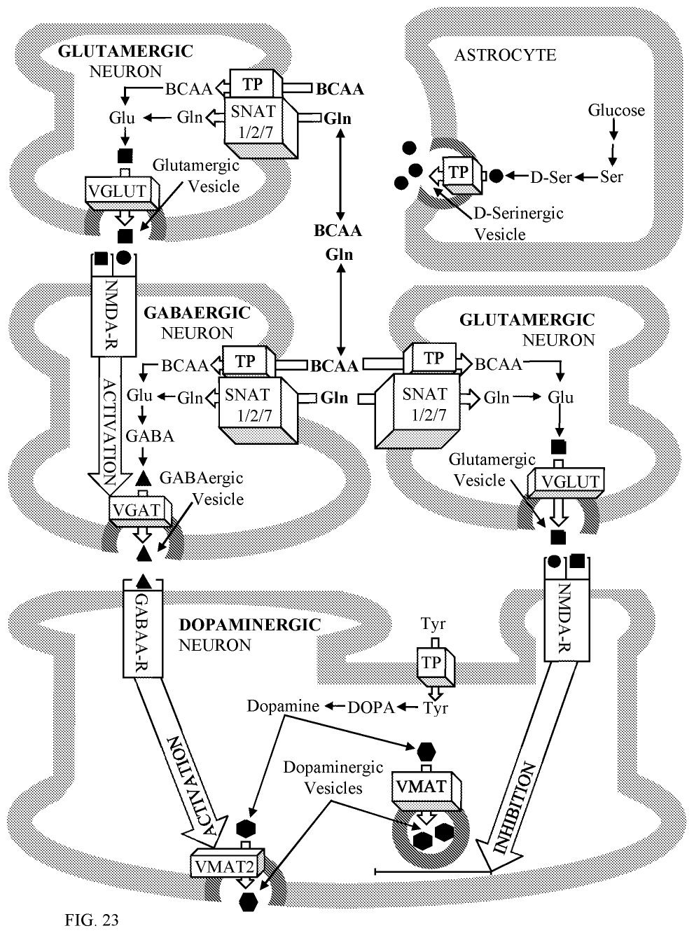

Also, an individual having a sexual dysfunction may discern the strength and duration of effect of a treatment substance (e.g., a new drug, a chemical from a biological source such as a plant, a fungi, a microorganism) for a sexual dysfunction and/or a neurotransmitter signaling pathway involved in another neurological disorder/injury whether located in the brain/peripheral tissue [e.g., spinal cord injury, Alzheimer's disease, amyotrophic lateral sclerosis, ataxia, bell's palsy, a brain tumor, a cerebral aneurysm, epilepsy, seizures, Guillain-Barre syndrome, schizophrenia, headache (e.g., tension headache, cluster headache, migraine headache), head injury, lumbar disk disease, meningitis, multiple sclerosis, muscular dystrophy, a neurocutaneous syndrome, Parkinson's disease, stroke, encephalitis, septicemia, a neuromuscular disease (e.g., muscular dystrophy), myasthenia gravis]. For example, FIG. 23 depicts a proposed model of schizophrenia wherein many of the same treatment substances described herein in promoting the synthesis neurotransmitters such as Glu, GABA, and dopamine, are involved in the neurotransmission pathways possibly causing schizophrenia. In the upper left, a Glu is synthesized from BCAA and released to activate a GABAergic neuron's NMDA-R receptor, and the NMDA-R sends an activation signal to promote the release of GABA, again synthesized from BCAA. The GABA activates a dopaminergic neuron's GABAA-R that sends an activation signal for the release of dopamine. However, another glutamatergic neuron, shown at the center right, can synthesize Glu from BCAA and release the Glu to activate a NMDA-R on the same dopaminergic neuron, and the NMDA-R sends an inhibition signal to reduce the amount of dopamine being released (shown as a bar before a dopaminergic vesicle). It is contemplated that a person with a sexual disfunction may be able to ingest a treatment substance and evaluate the strength and duration of the treatment substance's effect on the sexual dysfunction which may indicate the effectiveness of the treatment substance in treating another neurological disorder that uses the same neurotransmitters, receptors, enzymes, transporters, etc. (e.g., schizophrenia).

1-137. (canceled)

138. A composition for treating a reduced amount of tactile sexual function, comprising:
at least one positive GABAergic treatment substance, wherein the at least one positive GABAergic treatment substance increases the amount of a GABAA receptor agonist after the person ingests the at least one positive GABAergic treatment substance; and
at least one negative glutamatergic treatment substance, at least another positive GABAergic treatment substance, or a combination thereof;
wherein the at least one positive GABAergic treatment substance comprises at least one precursor of GABA, at least one GABA prodrug, at least one GABAA receptor agonist, or a combination thereof;
wherein the at least one negative glutamatergic treatment substance promotes the activation of the mGlu2 receptor, the mGlu3 receptor, or a combination thereof; and wherein the at least another positive GABAergic treatment substance activates a TRPV1-Receptor and activates a KCC2 transporter;
wherein the at least one positive GABAergic treatment substance and the at least one negative glutamatergic treatment substance, at least another positive GABAergic treatment substance, or a combination thereof, are in an effective amount to achieve pleasurable tactile sexual function just before the beginning of orgasm, just after the beginning of orgasm, or both, in a person.

139. The composition of claim 138, further excluding:
at least one negative GABAergic treatment substance, a positive glutamatergic treatment substance, or a combination thereof;
wherein the negative GABAergic treatment substance comprises a carbonic anhydrase inhibitor;
wherein the positive glutamatergic treatment substance activates at least one NMDA receptor, decreases KCC2 activity, or a combination thereof; and
wherein the at least one negative GABAergic treatment substance, the positive glutamatergic treatment substance, or the combination thereof is in an effective amount to reduce tactile sexual function, increase the time to ejaculation, or both.

140. The composition of claim 139, wherein:
the carbonic anhydrase inhibitor comprises apigenin, hesperidin, luteolin, mangosteen, spermidine, or a combination thereof; and
the positive glutamatergic treatment substance that activates at least one NMDA receptor, decreases KCC2 activity, or a combination thereof comprises NMDA.

141. The composition of claim 138, wherein:
the at least one precursor of GABA comprises at least one branched chain amino acid;
the at least one branched chain amino acid is selected from leucine, isoleucine, valine, or a combination thereof;
the at least one GABA prodrug comprises nicotinoyl-GABA;
the at least one GABAA receptor agonist comprises homotaurine;
the at least one negative glutamatergic treatment substance comprises at least one positive cysteineic treatment substance, noopept, or a combination thereof;
the at least one positive cysteineic treatment substance comprises N-acetyl-cysteine, cysteine, cystine; or a combination thereof; and
the at least one treatment substance that activates a TRPV1-receptor and activates a KCC2 transporter comprises piperine, vanillin, or a combination thereof.

142. The composition of claim 141; wherein:
the leucine is about 10.1 mg per kilogram body weight to about 116.1 mg per kilogram body weight;
the isoleucine is about 10.1 mg per kilogram body weight to about 48.4 mg per kilogram body weight;
the valine is about 10.1 mg per kilogram body weight to about 116.1 mg per kilogram body weight;
a combination thereof of leucine, isoleucine, and valine is about 60.7 mg per kilogram body weight to about 99.2 mg per kilogram body weight;
the nicotinoyl-GABA is about 1.5 mg per kilogram body weight to about 8.1 mg per kilogram body weight;
the homotaurine is about 0.7 mg per kilogram body weight to about 3.2 mg per kilogram body weight;
the N-acetyl-cysteine is about 3.7 mg per kilogram body weight to about 32.3 mg per kilogram body weight;
the cysteine, cystine, or both is about 7.4 mg per kilogram body weight to about 8.1 mg per kilogram body weight;
the noopept is about 0.4 mg per kilogram body weight to about 1.5 mg per kilogram body weight; and
the piperine is about 0.22 mg per kilogram body weight to about 0.24 mg per kilogram body weight.

143. The composition of claim 141, wherein the at least one branched chain amino acid consists essentially of valine.

144. The composition of claim 138, further comprising another at least one positive GABAergic treatment substance that:
increases the amount of a GABA receptor positive allosteric modulator;
increases the amount of a GABA receptor positive allosteric modulator and is a negative glutamatergic treatment substance;
inhibits a GABA transaminase, activates a glutamic acid decarboxylase;
inhibits a GAT transporter;
increases the amount of a GABA receptor agonist;
promotes a KCC2 transporter gene expression; or
a combination thereof.

145. The composition of claim 144, wherein the another at least one positive GABAergic treatment substance that:
increases the amount of a GABA receptor positive allosteric modulator comprises a Boswellia serrata preparation, a Crocus sativus preparation, a Piper methysticum preparation, theanine, a baicalin preparation, or a combination thereof;
increases the amount of a GABA receptor positive allosteric modulator and is a negative glutamatergic treatment substance comprises magnesium threonate;
inhibits GABA transaminase comprises a Melissa officinalis preparation;
activates glutamic acid decarboxylase comprises a Valeriana officinalis preparation;
inhibits a GAT transporter comprises taurine;
increases the amount of a GABA receptor agonist comprises GABA, a Withania somnifera preparation, or a combination thereof; and
promotes a KCC2 transporter gene expression comprises trans-resveratrol.

146. The composition of claim 145, wherein:
the Boswellia serrata preparation is about 3.7 mg per kilogram body weight to about 4.0 mg per kilogram body weight;
the Crocus sativus preparation is about 2.6 mg per kilogram body weight to about 2.9 mg per kilogram body weight;
the Piper methysticum preparation is about 4.3 mg per kilogram body weight to about 20.6 mg per kilogram body weight;
the theanine is about 5.9 mg per kilogram body weight to about 9.7 mg per kilogram body weight;
the baicalin preparation is about 4.0 mg per kilogram body weight to about 13.1 mg per kilogram body weight;
the magnesium threonate is about 9.8 mg per kilogram body weight to about 84.0 mg per kilogram body weight;
the Melissa officinalis preparation is about 16.5 mg per kilogram body weight to about 29.0 mg per kilogram body weight;
the Valeriana officinalis preparation is about 14.7 mg per kilogram body weight to about 35.5 mg per kilogram body weight;
the taurine is about 14.7 mg per kilogram body weight to about 16.1 mg per kilogram body weight;
the GABA is about 22.1 mg per kilogram body weight to about 24.2 mg per kilogram body weight;
the Withania somnifera preparation is about 4.4 mg per kilogram body weight to about 4.8 mg per kilogram body weight; and
the trans-resveratrol is about 7.4 mg per kilogram body weight to about 8.1 mg per kilogram body weight.

147. The composition of claim 138, further comprising at least one positive cholinergic treatment substance.

148. The composition of claim 147, wherein the at least one at least one positive cholinergic treatment substance comprises:
at least one acetylcholinesterase inhibitor;
at least one acetylcholine precursor; or
a combination thereof.

149. The composition of claim 148, wherein:
the at least one acetylcholinesterase inhibitor comprises a Panax genus preparation;
huperzine A, galantamine HBr, or a combination thereof; and
the at least one acetylcholine precursor comprises alpha-glycerophosphocholine, centrophenoxine, or a combination thereof.

150. The composition of claim 149, wherein:
the Panax genus preparation is about 8.8 mg per kilogram body weight to about 9.7 mg per kilogram body weight;
the huperzine A is about 0.00074 mg per kilogram body weight to about 0.00161 mg per kilogram body weight;
the galantamine HBr is about 0.59 mg per kilogram body weight to about 0.194 mg per kilogram body weight;
the alpha glycerophosphocholine is about 4.4 mg per kilogram body weight to about 4.8 mg per kilogram body weight; and
the centrophenoxine is about 7.4 mg per kilogram body weight to about 8.1 mg per kilogram body weight.

151. The composition of claim 149, wherein the at least one acetylcholinesterase inhibitor consists essentially of galantamine HBr.

152. The composition of claim 138, further comprising:
at least one positive cannabinoidergic treatment substance;
at least one positive nitroergic treatment substance;
at least negative adenosinergic treatment substance;
at least one positive glycinergic treatment substance; or
a combination thereof.

153. The composition of claim 152, wherein
the at least one positive cannabinoidergic treatment substance comprises a Syzygium aromaticum preparation, calcium pyruvate, oleamide, or a combination thereof;
the at least one positive nitroergic treatment substance comprises norvaline, icariin, or a combination thereof;
the at least one negative adenosinergic treatment substance comprises theobromine, caffeine, or a combination thereof; and
the at least one positive glycinergic treatment substance comprises glycine, pramiracetam, or a combination thereof.

154. The composition of claim 153, wherein:
the Syzygium aromaticum preparation is about 4.4 mg per kilogram body weight to about 4.8 mg per kilogram body weight;
the calcium pyruvate is about 13.2 mg per kilogram body weight to about 29.0 mg per kilogram body weight;
the oleamide is about 0.7 mg per kilogram body weight to about 3.2 mg per kilogram body weight;
the norvaline is about 3.9 mg per kilogram body weight to about 35.0 mg per kilogram body weight;
the icariin is about 0.88 mg per kilogram body weight to about 0.97 mg per kilogram body weight;
the theobromine is about 17.6 mg per kilogram body weight to about 19.4 mg per kilogram body weight;
the caffeine is about 1.5 mg per kilogram body weight to about 3.2 mg per kilogram body weight;
the glycine is about 14.7 mg per kilogram body weight to about 64.5 mg per kilogram body weight; and
the pramiracetam is about 3.7 mg per kilogram body weight to about 10.1 mg per kilogram body weight.

155. A method for treating a reduced amount of tactile sexual function, comprising:
ingesting a first composition in accordance with claim 138, and wherein tactile sexual stimulation occurs between about 5 minutes to about 120 minutes after ingesting the first composition.

156. The method of claim 155, further comprising:
ingesting at least another composition in accordance with claim 138 within 5 to 60 minutes of ingesting said first composition, and wherein tactile sexual stimulation occurs between about 5 minutes to about 120 minutes after ingesting said at least another composition.

157. A kit for treating a reduced amount of tactile sexual function and protecting a person from transmission of a sexually transmitted disease, an undesired pregnancy during sexual intercourse, or both, comprising:
at least one composition in accordance with claim 138; and
at least one condom, a wearable ring on a penis that comprises a vibrating devise, or a combination thereof.
